(19) United States
(12) Patent Application Publication
(10) Pub. No.: US 2010/0233677 A1
(43) Pub. Date: Sep. 16, 2010

(54) FULL GENOME SEQUENCES OF HUMAN RHINOVIRUS STRAINS

(76) Inventors: Stephen B. Liggett, Clarksville, MD (US); Claire M. Fraser-Liggett, Clarksville, MD (US); Ann C. Palmenberg, Madison, WI (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 12/703,147

(22) Filed: Feb. 9, 2010

Related U.S. Application Data
(60) Provisional application No. 61/151,302, filed on Feb. 10, 2009.

(51) Int. Cl. C12Q 1/70

(52) U.S. Cl. 435/5

(57) ABSTRACT

Infection by human rhinovirus (HRV) causes upper and lower respiratory tract disease with varying degrees of virulence. The molecular basis of diversity was examined by completing the genome sequences for all known serotypes (n=99) as well as novel field samples. Superimposition of capsid crystal structure and optimal-energy RNA configurations established the alignments. The phylogeny revealed conserved motifs, Glade-specific diversity including a potential new species (clade-D), pan-genome mutations in field isolates, and unexpected recombination that contributes to heterogeneity. A spacer tract near a 5′-UTR cloverleaf was hypervariable, and in analogy with poliovirus, may be associated with virulence. A previously unidentified configuration consistent with non-scanning internal ribosome entry may account for rapid protein translation. The data density from complete sequences of the HRV reference serotypes provided high resolution for this degree of modeling, and serves as a platform for full genome-based epidemiologic studies, for viral diagnostics and prognostics, and for antiviral compounds and vaccines.

### CROSS-REFERENCE TO RELATED APPLICATIONS

This application claims priority to U.S. Provisional Application No. 61/151,302, filed Feb. 10, 2009, which is herein incorporated by reference in its entirety.

### STATEMENT REGARDING FEDERALLY SPONSORED RESEARCH

This invention was made with U.S. Government support under National Institutes of Health Grant Number U19-A1070503. The U.S. Government has certain rights in this invention.

### BACKGROUND

1. Technical Field

This application relates generally to the determination of the complete genomic sequence of 80 novel human rhinoviruses and to the analysis of the sequence information from these novel sequences in conjunction with sequences information from other, known human rhinoviruses.

2. Background on Human Rhinoviruses

Human rhinovirus (HRV), the disease agent for the common cold, is responsible for ˜50% of asthma exacerbations and is one of the factors that can direct the infant immune system towards an asthmatic phenotype (J. M. Gwaltney Jr. et al., N. Engl. J. Med. 275, 1261 (1966); S. L. Johnston et al., Am. J. Respir. Crit. Care Med. 154, 654 (1996); K. G. Nicholson et al., BMJ 307, 982 (1993); D. J. Jackson et al., Am. J. Respir. Crit. Care Med. 178(7):667-72 (2008)). Direct and indirect costs from the common cold and related complications in asthmatics amount to an estimated ˜$60 billion per year in the U.S.A. (A. M. Fendrick et al., Arch. Intern. Med. 163, 487 (2003); K. B. Weiss et al., J. Allergy Clin. Immunol. 107, 3 (2001)). HRVs are single-stranded, positive-sense RNA Enteroviruses in the Picornaviridae family and have been catalogued primarily by capsid serotyping relative to a historical repository of 99 strains, obtained from clinical specimens. HRVs are classified by their use of either ICAM-1 (89 major viruses), or VLDL (10 minor viruses) as their receptor for cell entry (M. K. Cooney et al., Infect. Immun. 37, 642 (1982)). They have also been characterized by composite sensitivities across a panel of potential therapeutics (K. Andries et al., J. Virol. 64, 1117 (1990)) which have been used to parse the strains into two related drug-reactivity groups. The partial sequences of viral capsid-coding regions, non-coding regions, and a limited number of complete genomes have resulted in a division of the original 99 strains into two species: HRV-A (containing 74 serotypes) and HRV-B (containing 25 serotypes).

The naked RNA genome (no more than ˜8 kb in length) is surrounded by a capsid composed of 60 copies each of four structural proteins, denoted VP1-VP4, in an icosahedral configuration (20 triangular faces with 12 vertices). High resolution structural data reveal a star-shaped “plateau” at the 5-fold axis of symmetry, a deep depression (often termed the “canyon”) and a second protrusion at the 3-fold axis. The serotype used to classify HRVs is determined by the C-termini and connecting loops of the capsid proteins.

The HRV genome is translated as a single polyprotein, which is cleaved during translation by virus-encoded proteinases to produce 11 proteins (FIG. 5). The 5′-noncoding region exerts partial control of genome replication and translation, including a specialized motif which is an internal ribosome entry site (IRES) that directs mRNA translation by the host ribosomes. The 5′ end of the HRV genome is linked to a protein termed VPg (virion protein, genome linked) which is covalently linked at its third tyrosine residue by uridylylation. The VPg protein is virally derived from the cleaved encoded polyprotein. The 3′-UTR is highly structured and plays a role in replication and probably infectivity as well. HRVs attach to the host cell via receptors localized to the cells' surface membranes. The receptor binding site for those HRVs that bind to host-cell ICAM-1 is localized to the canyon. In contrast, it appears that the binding site for those HRVs that utilize the LDL receptor group lies at the small star-shaped dome on the icosahedral 5-fold axis (Hewat et al., EMBO J. 19(23):6317-25 (2000)). Just beneath the floor of the canyon of HRV-1a and HRV-16 is a hydrophobic pocket occupied by a lipid moiety. In contrast, the canyon hydrophobic pocket of HRV-14 is empty (Arnold et al., J. Mol. Biol. 211(4):763-801 (1990)). HRVs enter the cell by triggering receptor-mediated endocytosis, with uncoating occurring by endosomes. HRV replication requires viral RNA-dependent RNA polymerase, as well as multiple virus and host-cell derived accessory proteins. An intriguing aspect of picornavirus infections are their tendency for recombination within a cell. Poliovirus and foot-and-mouth disease virus have a recombination frequency estimated to be ˜0.9%. Since serotyping (or limited sequencing of a small region) is used in epidemiologic studies, there is no knowledge as to most of the sequence of the other HRVs, nor to the existence of additional HRVs, polymorphisms, or recombination events.

Recently, a number of new HRV-like sequences were detected in patients with influenza-like illnesses associated with severe respiratory compromise (S. R. Dominguez et al., J. Clin. Virol., 43, 219 (2008); P. McErlean et al., PLoS. ONE 3, e1847 (2008); S. K. Lau et al., J. Clin. Microbiol. 45, 3655 (2007)). The new viruses have not been cultured, but their sequences indicate they likely represent a third HRV-C species. The lack of whole-genome sequence data for the full cohort of HRVs has made it difficult to understand basic molecular and evolutionary characteristics of the viruses and has hampered investigations of the epidemiology of upper respiratory tract infections and asthma epidemics. The present invention addresses this lack of data.

### SUMMARY OF THE INVENTION

The present invention is based on the determination of the complete, novel genomic sequences of 80 human rhinoviruses (HRVs). These genomic sequences (SEQ ID NOs: 1-80) were obtained through the diligent efforts of the inventors as described herein and details concerning each of these novel sequences are provided in Table 1 (the novel sequences determined herein are marked with a double asterisk in column (1) of Table 1). The particular viruses from which the novel sequences were obtained include some of the 99 HRV serotypes maintained in the American Type Culture Collection (http://www.atcc.org) as well as 10 additional viruses obtained from field samples. The complete genomic sequences of the novel HRVs have been submitted to GenBank and the accession numbers of each of these sequences is provided in Table 1.

The present invention is further based on the analysis of the genomic sequence information obtained from the 80 novel HRVs in conjunction the published sequences of the additional 58 HRVs provided in Table 1. In a first aspect, the present invention is directed to use of this genomic sequence information in providing important and useful information concerning the structural and functional relationship between different HRV serotypes. In particular, a phylogenetic relationship based on the sequence information of the 138 HRVs listed in Table 1 was determined and is shown in FIG. 2, highlighting the relationship between the viruses. Further, a phylogenetic relationship between a subset of 55 of the HRVs was also determined based on genomic sequence data, the results of which are shown in FIG. 10. In addition, the amino acid sequences of the polypeptides encoded by the 80 novel HRV sequences were incorporated with the known amino acid sequence information of the other 58 HRVs of Table 1 to produce the amino acid sequence alignment shown in Table 5. This alignment highlights the differences and similarities between the proteins encoded by the viruses.

The structural and functional relationships between different serotypes can be used in a variety of manners including the following:

1. Development of a diagnostic test. The individual serotype sequences may form the basis for diagnostic tests for screening individuals for HRV infections, such as through the use of a gene chip containing all of the sequences in a known pattern. A sample from a subject can be applied to the chip to determine whether the subject is infected with a particular HRV serotype. Treatment can then be tailored to the specific serotype. The skilled artisan will understand that there are a variety of means to which the sequence information, and the structural and functional information based thereon, can be used in the development of diagnostic tests. As such, diagnostic testing is not limited to the use of gene chips.

In a similar fashion, consensus sequences determined through the genetic analysis discussed herein can also be used in the diagnosis and prognosis of HRV infection. For example, a consensus sequence can be included on a gene chip with diagnostic polynucleotides for a variety of different viral and/or bacterial infections. In this manner one chip can serve as the basis for diagnosing a whole range of infections. The skilled artisan will again understand that there is a variety of means to which the consensus sequence information, and the structural and functional information based thereon, can be used in diagnostic assays. As such, diagnostic testing is not limited to the use of gene chips.

2. Development of therapeutic agents to treat HRV infection. Information concerning structural and functional relationships between different HRV serotypes can be used in the development of therapeutics. Detailed knowledge concerning consensus sequences among serotypes, from both a functional and structural standpoint, can be used to facilitate the development of therapeutics that are effective against a large number of different serotypes. Similarly, treatments can be tailored to specific HRV serotypes based on unique properties discovered from the genomic sequence information, both polynucleotide and polypeptide sequences. Both the nucleic acid and amino acid sequences of specific serotypes, as well as consensus sequences, can be used, for example, in molecular modeling of small chemical agents that specifically target and bind the sequences, thereby disrupting the activity of the virus.

3. Development of vaccines to prevent HRV. The amino acid sequences of specific HRV serotypes provided herein can be used to prepare vaccines against specific serotypes. Similarly, information concerning structural and functional relationships between different HRV serotypes can be used in the development of vaccines. Consensus regions of the genomic sequence and proteins encoded thereby can be targeted in a vaccine development against a large number of different serotypes. Thus, while vaccines may be developed against specific serotypes, vaccines can also be developed against different groups of serotypes. Potentially, only a small number of vaccines would need to be included in one formulation to provide protection against most or all serotypes.

4. Following the progression of disease. There is growing evidence that asthma may be persistent if the HRV infection is slow to be, or never, cleared. Thus a test can be developed to monitor the titers of HRV from biological samples, such as nasal washes or bronchoscopy-derived fluids in patients with asthma or COPD. The results of such tests can be used to guide therapeutic decisions (e.g., anti-HRV or antibiotics for secondary infections). The knowledge gained from the genomic sequence information provided herein can be used in such tests.

Moreover, the genomic sequence information can be used to survey for mutations that occur among the general population of individuals that are being infected with HRV, such as through the use of the gene chips described above. Advance warning of mutations in particular regions can serve as the basis for developing vaccines and treatments to minimize the risk of an epidemic for serotypes causing more severe disease.

In a second aspect, the present invention is directed to the complete genomic sequence of the 80 newly sequenced HRVs (SEQ ID NOs: 1-80). These sequences are the complete genomic sequences of both previously described and newly discovered HRVs. The serotypes from which these 80 sequences were obtained and sequenced are marked with a double asterisk in the first column of Table 1. Thus, in one embodiment the present invention is directed to the genomic sequence of each of the 80 different HRVs marked with a double asterisk in the first column of Table 1. The GenBank accession number for the sequence information is provided in Table 1 as well (column (7)).

In a third aspect, the present invention is directed to the polypeptide sequences encoded by the genomic sequence of the 80 newly sequenced HRVs. As discussed herein, the genomic sequence of each HRV seroptype encodes 11 different proteins. Thus, in one embodiment the present invention is directed to each of the polypeptide sequences of each of the 80 newly sequenced HRVs. In a second embodiment the present invention is directed to polypeptide homologues having at least 95% amino acid sequence identity with each of the polypeptide sequences of each of the 80 newly sequenced HRVs. In a related embodiment, each of the polypeptide homologues has substantially the same activity as the polypeptide with which it is a sequence homologue. In a third embodiment, the present invention is directed to antibodies that specifically bind to the polypeptides and polypeptide homologues of the present invention. Preferably the antibody is a monoclonal antibody, a polyclonal antibody, or an active fragment of such an antibody.

In a fourth aspect, the present invention is directed to methods for assaying for the presence of an HRV serotype in a subject. The assay may be a diagnostic assay. The HRV serotype may be any HRV serotype, including those serotypes populated by the 138 HRVs set forth in Table 1. The diagnostic assay may be an assay for the presence of one or more HRV polynucleotide sequence, and/or the presence of one or more HRV polypeptide sequence. The assay may use a biological sample obtained from a subject. The method may be performed by obtaining a biological sample from a subject suspected of having an HRV infection and assaying the sample for the presence of one or more HRV polynucleotide sequences, and/or the presence of one or more HRV polypeptide sequences.

### BRIEF DESCRIPTION OF THE DRAWINGS

FIG. 1. Genome-wide optimal energy RNA configurations for select motifs representative of each HRV species. A) All HRV display characteristic 5′-UTR cloverleaf (CL) elements with minimal predicted structural variation (boxed sequences). B) The alignments and predicted structures of the IRES of HRVs reveal a bait-and-switch arrangement for initiation of translation. A stem-pairing AUG (light gray boxes) is found for each species, but a highly variable region of intervening sequence near the initiator AUG (dark gray boxes, white text) was noted. C) HRV 3′-UTRs have a unique unbranched stem motif prior to the poly-A tail. The left-most codon (dark gray boxes, white text) is the ORF terminator. Other boxes highlight additional terminators (light gray boxes), including a characteristic codon (light gray boxes, bold text) found in the apical loop.

FIG. 2. A neighbor-joining (NJ) phylogenetic tree showing relationships between all known HRV serotypes created on the basis of full genome sequences. The HEV-C sequences (poliovirus 1M, coxsackievirus a13, and coxsackievirus a21) were utilized as out-groups. Branch lengths are proportional to similarity (p-distance). Key nodes on this tree are annotated with NJ bootstrap values (% of 2000×). Asterisks (*) in the strain names identify sequences obtained from published data (Table 1). All other taxa are from this study. Letters in the outer rings designate whether that virus uses the major (M) ICAM-1 receptor, or the minor (m) VLDL receptor (M. K. Cooney, J. P. Fox, G. E. Kenny, Infect. Immun. 37, 642 (1982)), and whether its relative reactivity was more like group “1” or group “2” (if known) towards a panel of small-molecule antiviral compounds (K. Andries et al., J. Virol. 64, 1117 (1990)).

FIG. 3. Recombination of HRVs creates additional serotypes. A) Representative results showing that hrv-46 arose from a recombination of hrv-53 (major parent) and hrv-80 (minor parent). Shown are normalized pair-wise identities between each parent and the daughter hrv (“HRV-80 vs HRV46”; “HRV-46 vs HRV53”) and the two parents (“HRV-53 vs HRV80”). As indicated, hrv-46 nt 32-3222 are from hrv-80, and nt 3223-7200 are from hrv-53. B) Recombination with hrv-54-f05 has resulted in 7 serotype progeny. Each parental hrv is shown as a solid color. The contribution of each parent is proportional to the area of its color in the offspring. See Tables 1 and 3 for nucleotide boundaries and results from other recombination events.

FIG. 4. Whole genome amino acid sequence identity comparison. Amino acid sequences were deduced from the coding region of the sequenced Rhinovirus genome sequences. The aligned amino acid sequences were compared in a pairwise fashion to calculate the identities. The identity matrices of the compared genomes visualized in square arrays of sequence identity values were clustered by the phylogenetic ordering.

FIG. 5. An HRV genome map. A) The 5′ untranslated region (5′ UTR) is linked to VPg(3B), and encodes several important RNA structures which function during RNA synthesis and genome translation. The single open-reading frame (ORF) encodes a polyprotein, which is cleaved in a series of co-translational and post-translational reactions to provide all mature viral proteins required to establish and perpetuate an infection. The capsid proteins (1A-D) and proteins involved in replication functions (2A-C; 3A-D) are common to all HRV, and all sequences share analogous cleavage sites delineating these locations in the polyprotein. The illustrated genome is hrv-35; the base numbering system in this panel is for that sequence. B) The HRV sequences in the RNA genome alignment (data not shown) were queried pairwise at each position in the alignment. All sequences were given equivalent weight. The arithmetic average of the scores (0 or 1 for each pair) was reported for each position, re-averaged over a sliding window of 30 adjacent residues (+/−15), then plotted relative to the alignment as a whole. The strongest sequence conservation is in the 5′ IRES region. The lowest conservation is in the 5′ spacer region, upstream of the IRES, and also in the capsid coding regions, where the 1B, 1C and 1D troughs correspond to the respective, mapped immunogenic surface loops. The averaged identity across all HRVs, considering all alignment positions, is indicated with the horizontal dashed line.

FIG. 6A. Cloverleaf-like 2D RNA structures dominate the 5′ ends of all HRV genomes. A representative structure for each species is depicted. The cloverleafs bind host and cellular proteins required for the initiation of viral RNA synthesis and also aid in the regulation of ribosome entry onto the upstream IRES (internal ribosome entry site). Adjacent to the cloverleaf all HRV have a pyrimidine-rich tract. In related polioviruses, this tract binds PCBP2, a cellular factor needed for viral RNA synthesis and IRES-dependent translation. However, the specific sequence in this region is virtually unique to every known isolate of HRV, varying even among field-strains of the same serotype. Examples of the length and sequence diversity among HRVs are shown for each genus (gray boxed areas). The flanking sequences (labeled) are alignment landmarks, common to all HRVs. The 5′-most nucleotide of every picornavirus is “U” and it is linked by a tyrosine-phosphodiester bond to viral protein VPg (3B).

FIG. 6B. The RNA structures near the AUG which begins the polyprotein open-reading frame (ORF) are depicted for representatives of each HRV species. Every sequenced HRV folds this region into a similar configuration when the genome is probed by minimum-free energy calculations. The ORF AUG (white letters, dark gray boxes) is always paired with a conserved upstream AUG (light gray boxes), which marks the 3′ boundary of the IRES. Other species in the Enterovirus genus do not make similar pairings. Rather, other enteroviruses are believed to launch their ribosomes for scanning, from the upstream AUG. This HRV configuration makes it likely that an IRES-bound ribosome could readily “switch” to the ORF AUG, without scanning the intervening spacer. Between these AUGs, neither the specific sequence or its length is well conserved among HRVs. Rather, all form these related structures, preserving the specific pairings only in the region of the AUGs.

FIG. 6C. Picornavirus RNA synthesis is protein primed. The viral RNA polymerase 3Dpol, uridylylates protein 3B (VPg) to form VPg-pUpU, then uses this protein:RNA as primer for the initiation of positive and negative strand RNA synthesis. The uridylylation reaction is templated by a special RNA structure called the cre (cis-acting replication element) whose location varies for every known species of picornavirus. For the HRV-A, the cre has been mapped within the genome region encoding the 2A protein. For the HRV-B, the cre is within the 2C region. The HRV-C cre has not been mapped genetically, but common to all cres, is a distinct stem motif, displaying CAAACAA or a closely related sequence. Within the sequenced genomes of the HRV-C there are 2 locations in the 1B gene which fit this description. These, and the HRV-A and HRV-B cres are depicted. The structure is for the indicated sequence. Those bases marked with a dot are conserved among all other sequences in that species. All observed (aligned) base changes are underlined. Below each element is the protein sequence encoded by that region. Residues marked with a dot are conserved among all sequences in that species. Underlined residues are observed in at least 1 other sequence. Note: the HRV-A structure omits sequence variation contributed by the clade-D viruses. It is not yet clear whether the cre for these viruses occupies the same genome location.

FIG. 6D. Representative HRV genomes were analyzed for their minimum free-energy configurations. Within those datasets, the regions representing the 3′ untranslated regions (3′UTR) fold into motifs independent of the rest of the genome. Examples of these regions, typical of each HRV species are illustrated (ORF terminator, dark gray boxes, white letters). In every case a single stem motif dominates the region, ending exactly at the 3′ poly(A) tail, which may contribute 1-2 additional base-pairs to the bottom of the stem. Sequence conservation is poor throughout this region, but every virus, with the exception of the clade-D isolates, displays a termination codon within the terminal loop of the 3′ stem (light gray boxes marked with an asterisk). Although multiple other termination codons are scattered frequently throughout the region (light gray boxes), this particular triplet is the only one which is conserved among most of the isolates, regardless of species. The clade-D viruses (e.g. hrv-95) have shorter 3′ stems and display an UAG triplet near, but not within the terminal loop (FIG. 7).

FIG. 7. Among the HRV-A, 3 serotypes (hrv-08, hrv-45 and hrv-95) form a distinct Glade, called “clade-D”. Of these, hrv-08 and hrv-95 share >98% nucleotide identity, perhaps indicating a misidentification within the original reference collection. Despite the paucity of unique isolates in this Glade, the identified members share characteristics in certain key genome locations which clearly distinguish them from all other HRV-A sequences (or HRV-B and HRV-C). A few examples are shown in this figure. A) Near the C-terminus of the 2A protein, in a region known to contribute to substrate specificity, all clade-D viruses have an indel unique among all other HRV. B) Throughout the alignments, there are numerous locations, like this one in the 3Dpol gene, where clade-D sequences vary independently with highly variant, non-synonymous substitutions. C) The cre elements of all HRV-A are located 2A gene (see FIG. 6C). The loop sequences, especially in the essential terminal loop invariably display ACAAACAA motifs providing the template for 3D-dependent VPg uridylylation. The “G” nucleotide (boxed) displayed within the clade-D loop has not been observed in any other functional cre element. This may indicate that for this Glade, the (authentic) cre lies elsewhere in the genome, or that these particular cres have a different templated activity. D) The 3′ UTR sequences of all HRVs, with the exception of the clade-D viruses, display a conserved termination codon in the terminal 5-6 b stem loop (FIG. 6D), and moreover, there are very few examples of extended interior loops within the terminal stem. The clade-D viruses pair the conserved termination codon, near the top of the stem, and display a second, unpaired codon, within an interior loop (light gray boxes, bold letters). They are also the only sequences to have just 4 b in the terminal loop. This entire 3′ configuration is unique to this Glade, and not shared by any other HRV-A, HRV-B or HRV-C.

FIG. 8. The distribution of amino acid variations in field samples. Each field sample was aligned with the reference sample of the same serotype. Amino acid differences in the field sample are designated by color changes as identified in the key at the bottom of the figure. The upper panel portrays a diagram of hrv-14 genome structure for orientation.

FIG. 9A. The RNA genome alignment (data not shown) was augmented with additional outgroup sequences from the HEV-C species (pv21:M12197, pv31:K01392) and the HEV-B species (cvb1:M16560, cvb2:AF081485, cb3:M33854), with profile:profile fits in Clustal. A maximum likelihood (ML) tree was calculated using PHYML version 2.4.8 for the full genome dataset (“full”). TN93 nucleotide substitution model gave the best likelihood (loglk-462223) over HKY, GTR, JP69, K2P and F81 models. The Ts/tv ratio and invariable sites were estimated. Approximate likelihood ratio tests (aLRT, minimum of SH-like and Chi2-based values) and bootstrap tests (BS, 200×) were applied. The test values label the tree nodes (ratios expressed as % BS:aLRT). Tree length (sum of branch lengths) is 19.39. When compared to the FIG. 2 neighbor-joining (NJ) tree, terminal clades were collapsed if they were identical to the NJ tree. Of all considered linkages, only hrv78 and the hrv(31+47) Glade showed minor rearrangements by ML analyses relative to NJ (thin line branches). Parallel ML trees were also calculated using only the VP1, VP0, IRES or 3D-encoding RNA sequences (see Methods). Taxa names with different Glade affiliations (>0.2 p-value) on the VP0-only tree are marked with a dot. Taxa names with different Glade affiliations on VP1-only tree are boxed.

FIG. 9B. The serotype of an HRV is determined by sequences within the immunogenic surface loops of capsid proteins VP0 (VP4+VP2) and VP1. Frequently, these segments and/or the IRES and/or the 3D pol regions are sequenced separately and used to place new isolates onto HRV species trees. Established statistical methods within the program set CONSEL (V0.1i) tested the null hypothesis that the topology of such “serotype only” trees, or an IRES-only tree, or a 3D-only tree were consistent with the FIG. 9A full genome ML tree. The program output includes: the observed log-likelihood difference (obs); the p-value of the approximately unbiased test calculated from the multiscale bootstrap (au); the bootstrap probability calculated from multiscale bootstrap (np); the bootstrap probability calculated in the usual manner (bp); the Kishino-Hasegawa test (kh); the Shimodaira-Hasegawa test (sh); the weighted Kishino-Hasegawa test (wkh); and the weighted Shimodaira-Hasegawa test (wsh). The last 6 tests had identical output values for all considered trees, and are summarized together. The data, individually and collectively strongly reject the null hypotheses for all alternative tested topologies.

FIG. 9C. The five specific maximum likelihood (ML) tree topologies tested in FIG. 9B are indicated.

FIG. 10. Phylogenetic relationship between 55 of the novel HRV sequences.

### DETAILED DESCRIPTION OF THE INVENTION

The present invention provides the complete, novel genomic sequences of 80 HRVs (SEQ ID NOs: 1-80). Description information concerning these sequences is provided in the five tables included herein, with Table 1 providing the GenBank accession number (column (7)) for each of the different genomic sequences. Of the 138 different HRVs, 80 of the genomic sequences are novel (those HRVs marked with a double asterisk in column (1) of Table 1).

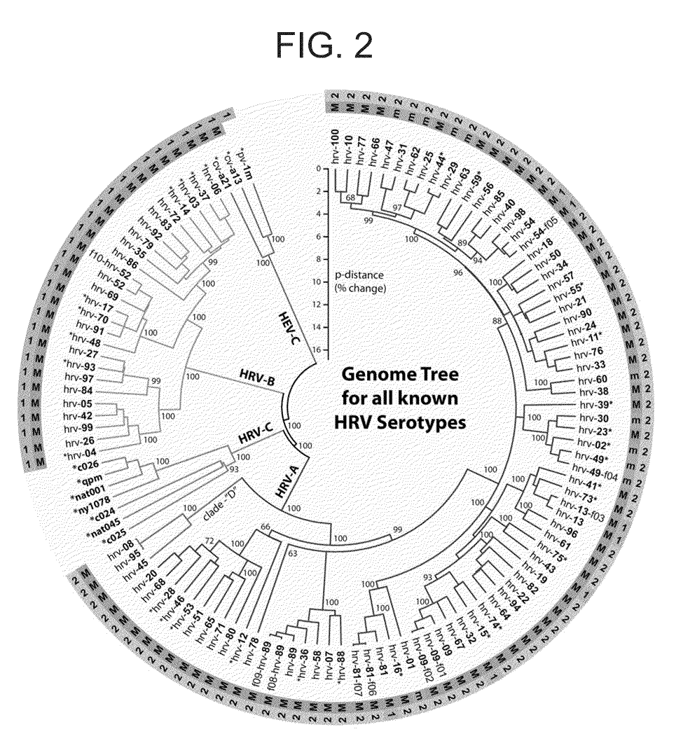

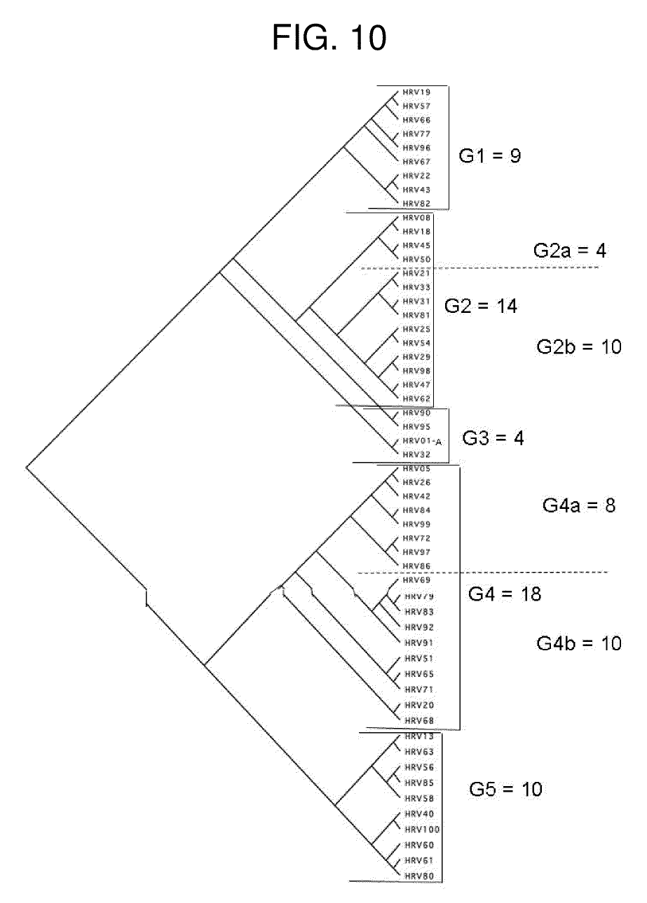

As discussed above, the genomic sequence information of each of the 138 different HRVs forms the basis for each aspect of the invention. The present invention is directed to use of the genomic sequence information in providing important and useful information concerning the structural and functional relationship between different HRV serotypes. In particular, a phylogenetic relationship between all known HRV serotypes based on genomic sequence data was determined and is shown in FIG. 2. Further, a phylogenetic relationship between a subset of 55 of the novel HRVs discussed above was also determined based on genomic sequence data, the results of which are shown in FIG. 10.

In a first aspect, the structural and functional relationships between different serotypes can be used in a variety of manners including: (i) development of diagnostic and prognostic tests for HRV infection, (ii) development of therapeutic agents for use in the treatment of HRV infections, (iii) development of vaccines to prevent HRV infection, and (iv) serving as a basis from which to follow the progression of disease in a subject or a population of subjects.

The skilled artisan will readily understand the wide variety of uses to which the genomic sequence information, and the structural/functional information produced from the sequence information, can be put. Without being bound by a specific group of assays, procedures or techniques, polynucleotide and polypeptides sequences, both those of specific HRV serotypes and HRV consensus sequences, can be used in gene chips for diagnostic tests, for assaying potential HRV therapeutics and vaccines, and to monitor HRV infections in individuals. Similarly, the polynucleotide and polypeptides sequences can be used in affinity columns, Southern blots, western blots and northern blots, and to design primers for whole genome sequencing of HRVs.

In a second aspect, the present invention is directed to the complete genomic sequence of the 80 newly sequenced HRVs. The serotypes from which these 80 sequences were obtained and sequenced are marked with a double asterisk in the first column of Table 1. Thus, in one embodiment the present invention is directed to the complete genomic sequence of each of the 80 different HRVs marked with a double asterisk in the first column of Table 1. The GenBank accession number for the sequence information is provided in Table 1 as well (column (7)).

In a related aspect, the present invention is directed to the individual open reading frames (ORF) contained within the 80 different genomic HRVs sequences. As discussed above, each of the single stranded genomic sequences encodes 11 different proteins. The specific boundaries of the ORFs are provided in Tables 1 and 3. The skilled artisan will readily understand how to determine the specific ORF of each of the genomic sequences.

The present invention further comprises polynucleotide homologues of each of the HRV ORFs. As used herein, the polynucleotide homologues of the present invention include those polynucleotides having at least about 70%, 75%, 80%, 85%, 90%, 91%, 92%, 93%, 94%, 95%, 96%, 97%, 98% or 99% sequence identity to any of the HRV ORFs encompassed within the genomic sequences of the HRV serotypes included herein. In one embodiment the polynucleotide homologues encode polypeptides that have substantially the same activity as the polypeptides encoded by the polynucleotides with which they are homologous (i.e., the wild-type polynucleotide). As used herein, substantially the same activity means about 100%, 99%, 98%, 97%, 96%, 95%, 90%, 80%, 70%, 60%, 50%, 40%, 30%, 20%, or 10% of the activity of the wild-type polypeptide.

In a third aspect, the present invention is directed to the polypeptide sequences encoded by the genomic sequence of the 80 newly sequenced HRVs. As discussed herein, the genomic sequence of each HRV seroptype encodes 11 different proteins. Thus, in one embodiment the present invention is directed to each of the polypeptide sequences encoded by each of the ORFs of the 80 newly sequenced HRVs. An alignment of the polypeptide sequences of the 80 newly sequenced HRVs, along with the previously disclosed polypeptide sequences of the other HRVs shown in Table 1, is provided in Table 5.

In a related aspect the present invention is directed to polypeptide homologues of the polypeptide sequences of each of the 80 newly sequenced HRVs. As used herein, the polypeptide homologues of the present invention include those peptides and polypeptides having at least about 70%, 75%, 80%, 85%, 90%, 91%, 92%, 93%, 94%, 95%, 96%, 97%, 98% or 99% sequence identity to any of the HRV peptides or polypeptides described herein. In a preferred embodiment the polypeptide homologues have substantially the same activity as the polypeptide to which they are homologous (i.e., the wild-type polypeptide). As used herein, substantially the same activity means about 100%, 99%, 98%, 97%, 96%, 95%, 90%, 80%, 70%, 60%, 50%, 40%, 30%, 20%, or 10% of the activity of the wild-type polypeptide.

In further related aspect, the present invention is directed to antibodies that specifically bind to the polypeptides and polypeptide homologues of the present invention. Preferably the antibody is a monoclonal antibody or a polyclonal antibody, including chimeric and humanized monoclonal and polyclonal antibodies, or an active fragment of such an antibody. Active fragments include single chain antibodies, Fab fragments, F(ab)₂ fragments, The skilled artisan will readily understand how to produce the antibodies of the present invention.

In a fourth aspect, the present invention is directed to assays for the presence of an HRV serotype in a subject. The assay may be a diagnostic or prognostic assay. The HRV serotype may be any HRV serotype, including those populated by the 138 HRVs set forth in Table 1. The diagnostic or prognostic assay may be an assay for the presence of one or more complete or partial HRV genomic sequence, including specific ORFs or portions thereof, and untranslated regions or portions thereof. The diagnostic assay may further be an assay for the presence of one or more HRV polypeptide sequences, or one or more portions of an HRV polypeptide sequence. The assay may use a biological sample obtained from a subject. As used herein, the biological sample obtained from a subject may be any biological sample that could contain some amount of an HRV polynucleotide or HRV polypeptide. The biological sample can be, for example (but is not limited to), nasal wash, nasal discharge, nasal scraping, nasal epithelial sampling (q-tip, spatula, etc.), tracheal wash, bronchial wash/lavage, bronchial epithelial biopsy, bronchial epithelial brushing, bronchial smooth muscle biopsy, bronchoalveolar lavage or lung biopsy. As used herein, “subject” is intended to mean an animal, such as mammals, including humans and animals of veterinary or agricultural importance, such as dogs, cats, horses, sheep, goats, and cattle. The biological sample can also be from tissue culture cells that have been infected with a biological sample from a subject, which allows-for replication of the HRV to produce larger amounts of virus for subsequent studies.

In other aspects of the present invention the HRV genomic sequence information, and the structural and functional information derived therefrom, both that described herein and that later developed from the sequence information, can be used in the diagnosis, prognosis, treatment, and surveying of a number of different diseases including: rhinitis, sinusitis, common cold, upper respiratory tract infection, otitis media, asthma, pneumonia, asthma (where HRV is a causative agent), asthma exacerbation (where HRV infection is not the causative agent but instead exacerbates the condition), persistent asthma (where uncleared HRV infection causes persistent asthma), and chronic obstructive pulmonary disease (COPD) exacerbation (where HRV infection is not the causative agent but instead exacerbates the condition). That is, the HRV genomic sequence information, and the structural and functional information, can be used in developing screening assays, means for treatment and therapy, means for prevention and prophylaxis, and means for monitoring disease progression in a number of diseases caused by or related to different HRV serotypes.

Thus, the skilled artisan will readily understand and appreciate that the genomic sequence information, and the structural and functional information derived therefrom, can be used in a large number of different manners, both those contemplated and discussed herein, and those that may be later developed.

I. HRV Genome Sequencing

To define the extent and nature of human rhinovirus (HRV) diversity and evolution, the genomes for every previously HRV that did not have full genome sequence in the reference repository were sequenced, completing the full set of 99 serotypes, as well as 10 new field samples (Table 1). Full (or complete) genome sequences is defined as the contiguous sequence without gaps from the first (or nearly first) nucleotide to the last (or nearly last) of the HRV genome. Modifications (A. Djikeng et al., BMC Genomics 9, 5 (2008)) were made to the sequence-independent, single-primer amplification (SISPA) method (see below) in order to determine the complete genomes of 70 HRVs from the reference repository, as well as 10 nasal wash samples from patients with HRV upper respiratory tract infections. The viruses were sequenced at an average of 6× complete coverage for each of the ˜7 kb genomes.

II. HRV Genome Analysis

To provide accuracy for determining the differences and commonalities between HRVs, and the phylogenetic relationship between these organisms with relatively small genomes and (often) high degrees of sequence similarity, a stringent approach was taken for aligning the sequences. The initial sequence fits for the polyproteins were performed on the basis of superimposition of the amino acid sequences within virion crystal structure maps (A. C. Palmenberg, J.-Y. Sgro, in Molecular Biology of Picornaviruses, B. L. Semler, E. Wimmer, Eds. (ASM Press, New York, 2001)) and supplemented with additional structure data from other viral proteins. In a step-wise manner, profile hidden Markov models (HMM) augmented the founder set with the remaining sequences (see below). The published sequences (including redundant determinations) for the remaining serotypes were added so that the final collection consisted of 138 full-length HRV genomes, including at least 1 representative for each of the 99 original strains, 10 field samples, and 7 HRV-C strains (Table 1).

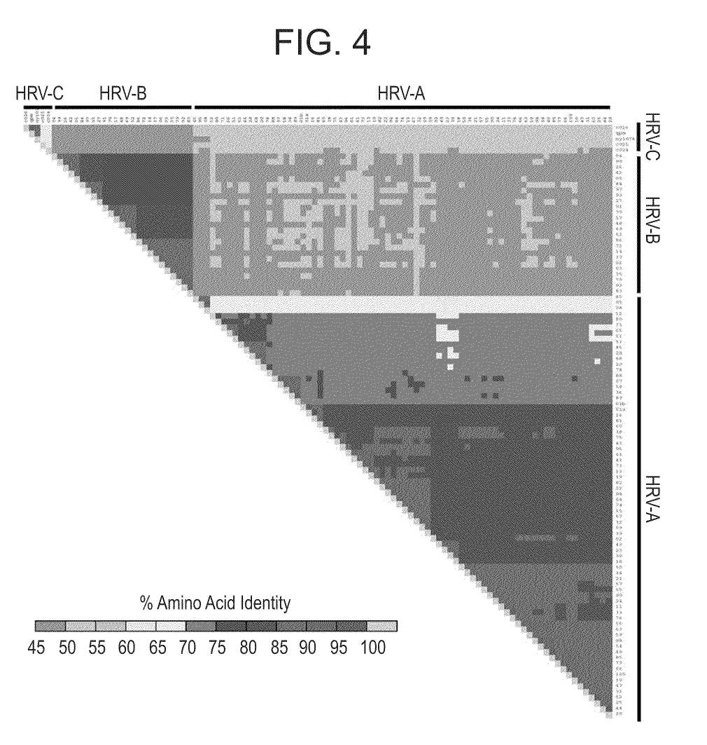

Regardless of species, all HRVs were found to have similar average base compositions. They are rich in A (31-34%) and U (25-30%), but low in G (19-22%) and C (18-22%). The third codon positions have the highest composition skew. An identity matrix prepared for the polyproteins (FIG. 4) shows that the average amino acid identity between pairs of HRV-C strains were slightly more diverse (78% identity, range 68% to 95%), than among the HRV-A (80%, range 64% to 99%) or HRV-B (83%, range 75% to 97%). Table 5 provides an amino acid sequence alignment of each of the serotypes set forth in Table 1. The lack of broader diversity suggests all HRVs are in a stable status for maintaining selection for certain traits, yet still have mutational flexibility for escape from immune responses.

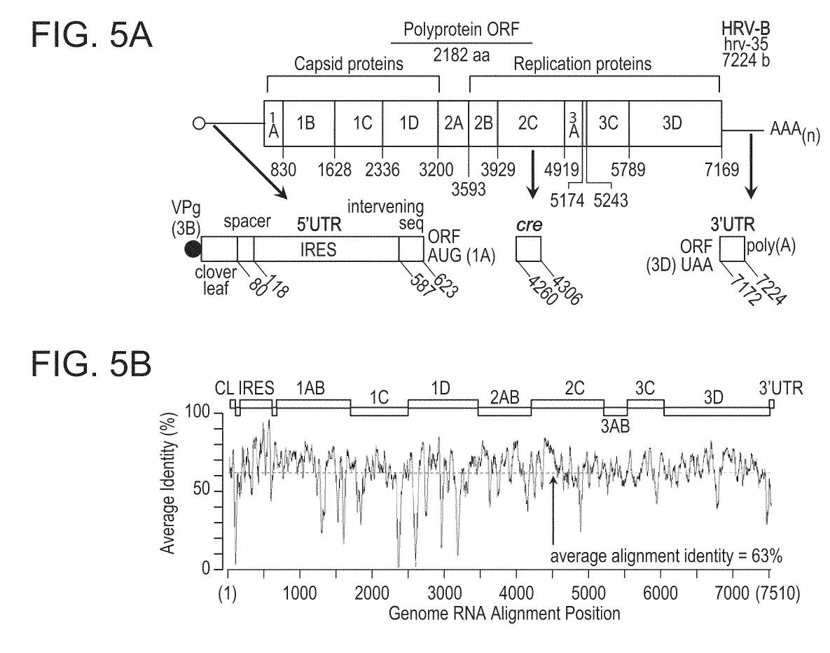

HRVs encode a single open reading frame (ORF) representing about 90% of the RNA length. Translation produces a polyprotein that is subsequently cleaved in a viral protease-dependent cascade to form the 11 mature viral proteins required to initiate and sustain an infection (FIG. 5A). Local and global RNA structures play established roles in HRV biology, however the extent, character, and relatedness, of serotype-specific 5′- and 3′-UTR variation are unknown. The role of this variability has thus not been related to function by modeling techniques or in vivo approaches. The alignment methods herein included optimal energy RNA structure considerations (A. C. Palmenberg, J.-Y. Sgro, Seminars in Virology 8, 231 (1997)) and were therefore sensitive to potential differences amongst the HRVs in the 5′- and 3′-UTRs.

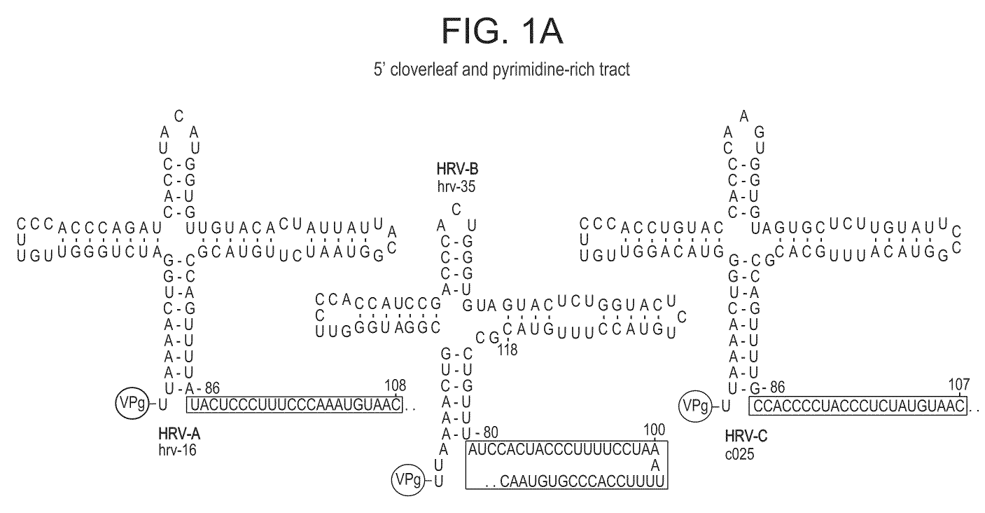

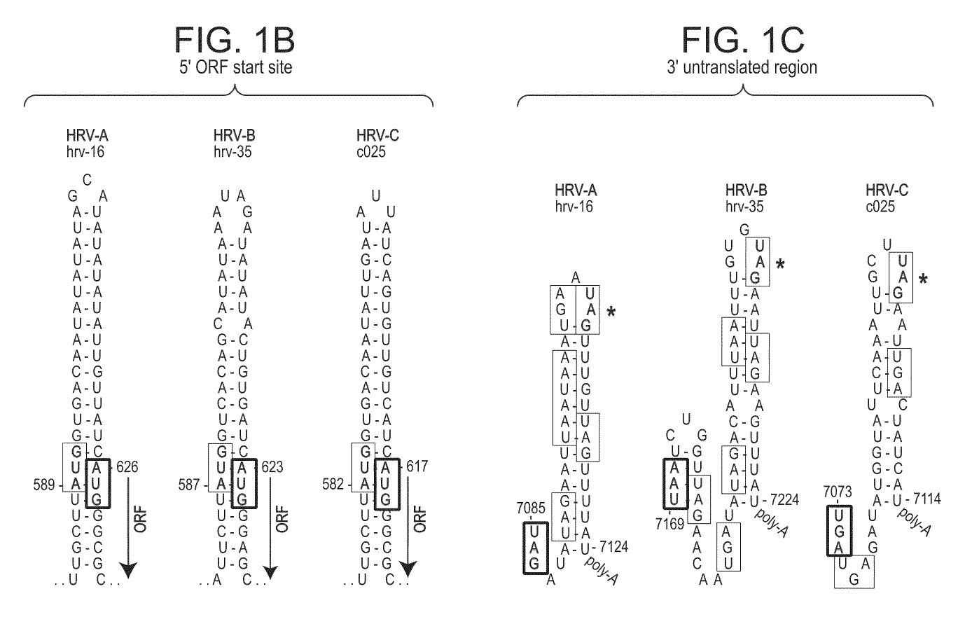

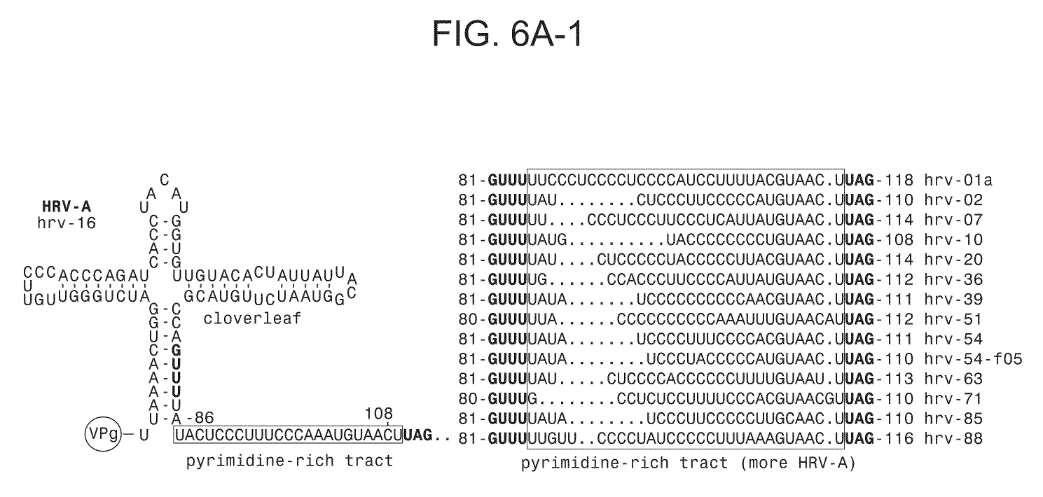

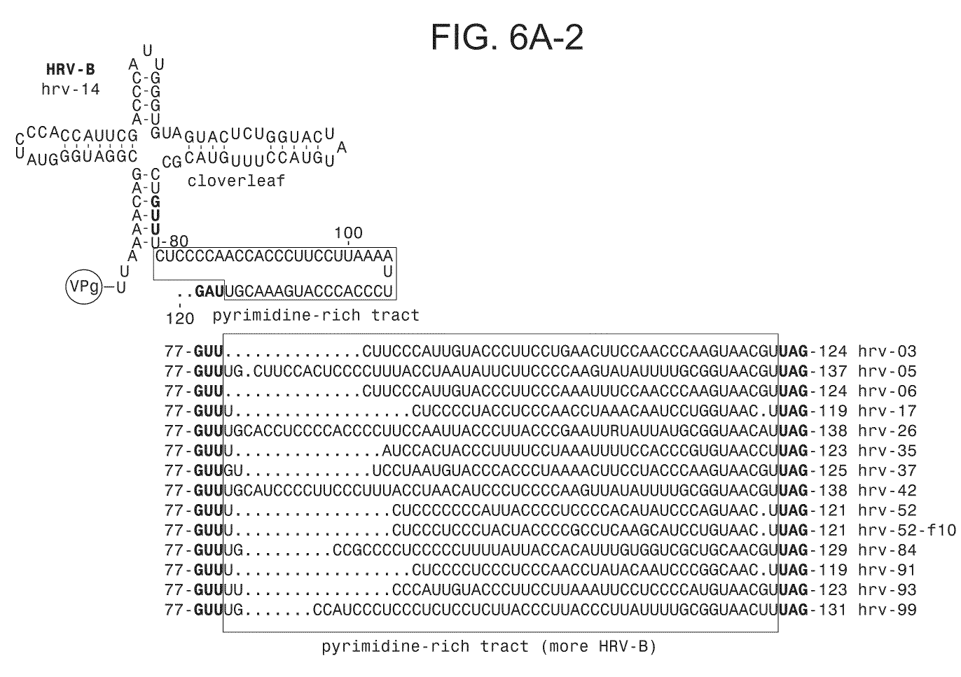

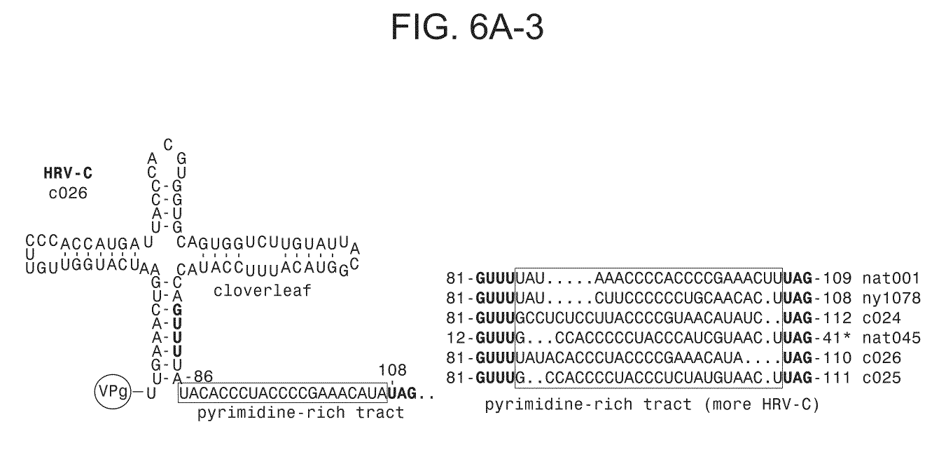

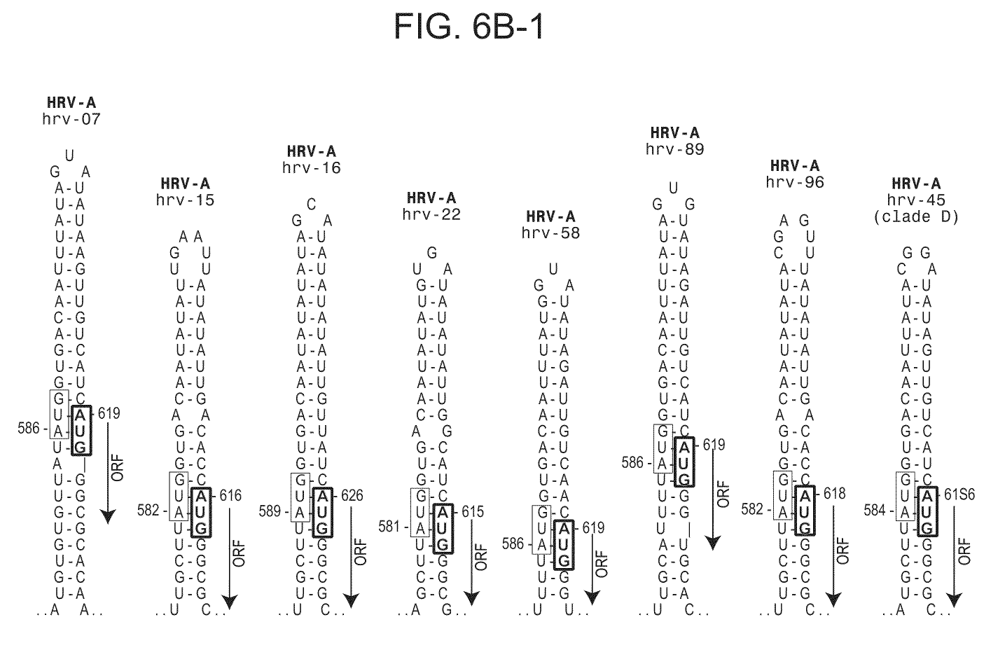

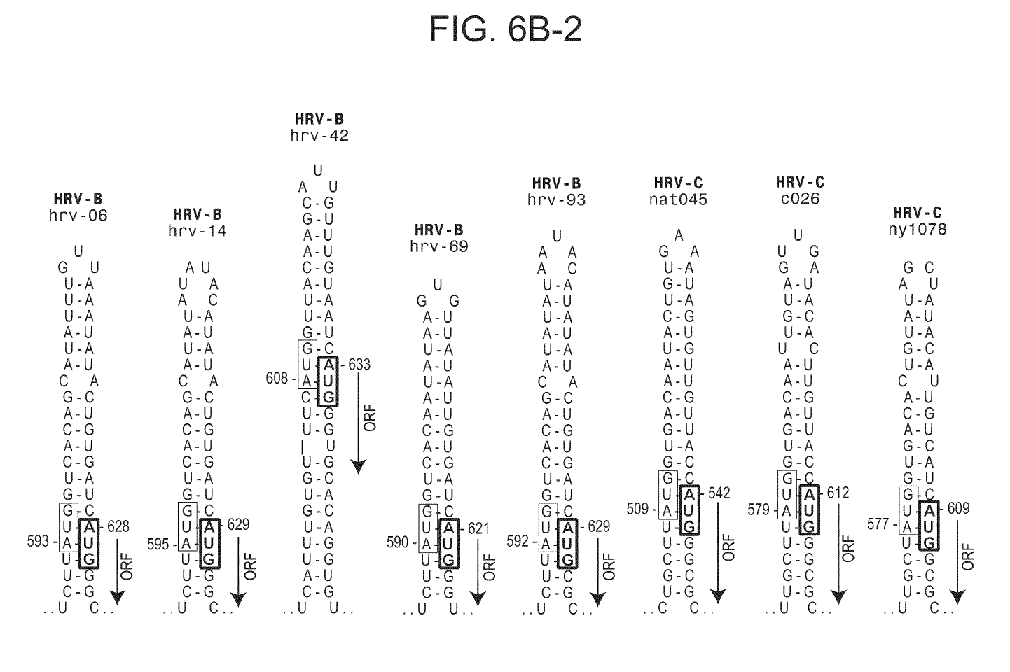

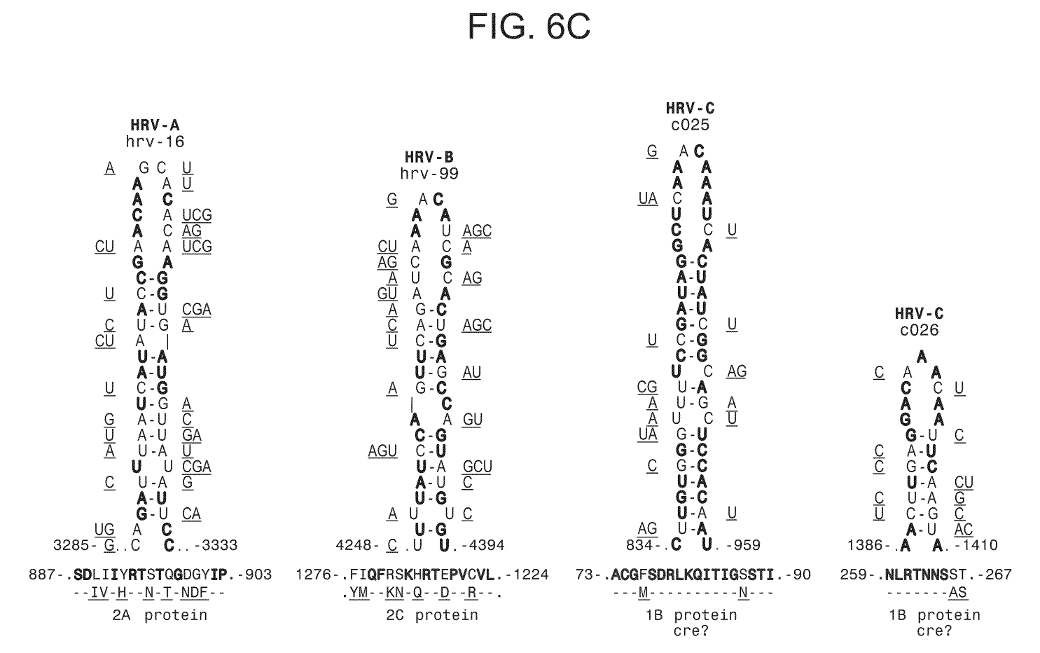

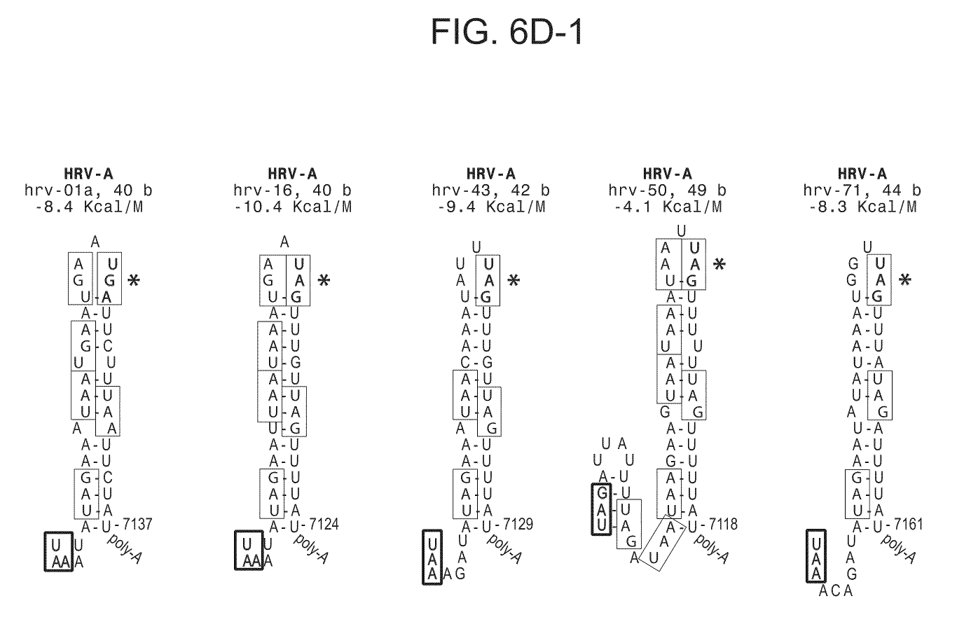

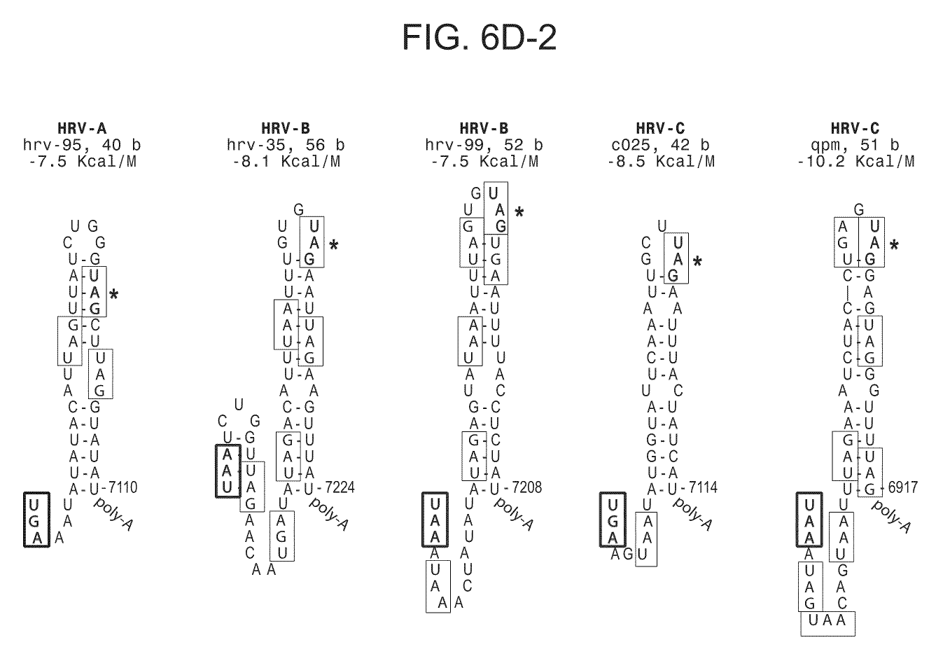

HRV RNA Structures. All enteroviruses encode 5′-terminal cloverleaf-like motifs (CL) which bind viral and cellular proteins for the initiation of RNA synthesis and also help convert infecting genomes from translation to replication templates. The HRV CLs (80-84 b) were predicted in every sequence with minimal structural variation among the species (representative structures are shown FIG. 1A and additional structures in FIG. 6A). Immediately 3′ to the CL, all HRVs were found to share an unusual pyrimidine-rich spacer segment with short oligo(C) and oligo(U) units interspersed with As (gray boxes, FIG. 1A and FIG. 6A). The HRV-A have the shortest tracts (11-22 b) and HRV-B the longest (22-50 b). Nearly every HRV displayed a unique sequence in this region, and unexpected variations were identified even among isolates of the same serotype (FIG. 1A and FIG. 6A). The equivalent genome location in poliovirus (10 b) interacts with poly(C)-binding protein 2 and is involved in the determination of the polio neurovirulent potential. The analogous regions in aphthoviruses or cardioviruses have homopolymeric poly(C) or poly(UC) tracts, the deletion of which significantly attenuates the virus through a PKR activation-dependent mechanism (L. R. Martin, G. M. Duke, J. E. Osorio, D. J. Hall, A. C. Palmenberg, J. Virol. 70, 2027 (1996)). If the HRV 5′ spacer tracts are functional analogs to those of these other picornaviruses, then it is possible that the pathogenic potential of an individual HRV may also be encoded, in part, by this region.

Picornaviruses use internal ribosome entry sites (IRES) to mediate translation initiation of their polyprotein ORFs. The IRESes of all enteroviruses (termed Type-1 IRESes) are thought to bind 40S subunits internally within their 5′-UTR, and to then scan additional nucleotides to find the proper initiator AUG (R. J. Jackson, in Translational Control, W. B. Hershey, M. B. Mathews, N. Sonenberg, Eds. (Cold Spring Harbor Laboratory Press, 1996)). Modeling of the known serotypes confirmed that all HRV IRESes start just 3′ to the pyrimidine-rich spacer tract. The internal IRES sequences were found to be highly conserved with an average nucleotide identity of 82%. Indeed, this region of the genome has the greatest degree of identity among all HRV (FIG. 5B), exceeding >95% for regional motifs within the IRES. However, it was also observed that the dominant IRES sequence conservation did not extend completely to the initiator AUG and for that the 18-40 bases 5′- to this codon, the region scanned by ribosomes, there was little species-specific conservation (<60% nucleotide identity). Despite this, the folding predictions configured every one of these regions into virtually the same RNA motif suggesting that this structure is conserved even when the underlying nucleotides are not (FIGS. 1B and 6B).

Near the bottom of a long (15-20 bp) minimum energy unbranched stem, the ORF AUG was invariably paired with a conserved upstream non-coding AUG (light gray boxes, FIG. 1B), marking the 3′ boundary of the IRES, and the normal launch point for 40S scanning. Every HRV genome fold maintains this pairing. It was predicted that HRVs use the proximity of these AUGs to orient the 40S for direct transfer to the proper codon, without the need for scanning through the intervening nucleotides. The sequence and length variation between the AUGs was consistent with the idea that ribosomes would by-pass this region entirely, if they jump from one AUG to the other. This IRES folding is essentially a bait-and-switch mechanism which was predicted to potentially enhance HRV translation competitiveness. This paired AUG motif is unique to HRV and is not found in other enteroviruses (e.g., poliovirus).

The HRV 3′-UTRs (40-60 b) begin with the ORF termination codon and extend to the genetically-encoded poly(A) tail. The ORF terminators themselves (dark gray boxes in FIG. 1C and FIG. 6D) included UAG, UAA and UGA codons. The codon selection often differed among isolates from the same serotype. Multiple additional terminators (light gray boxes), in and out of the ORF, were identified that punctuated each segment (3-9 per UTR). Despite large differences (>40%) in nucleotide identity (FIG. 1C), it was noted that all HRV maintain a 13-16 by unbranched stem, covering 67-88% of the 3′-UTR, immediately abutting the poly(A) tail (see also FIG. 6D). Some 3′ stems have small interior loops, but nearly all, except for the unusual sequences of clade-D (see below), present 5-base terminal loops, anchored with apical U-G or U-A pairs. Inevitably, the 3′ sides of these terminal loops display UAG or UGA terminator codons, which may or may not synchronize with the local ORF. The function of these 3′ stems is unknown, but such conservation is usually indicative of a putative protein recognition motif (such as translation termination factors), or alternatively, an RNA:RNA tertiary interaction between the terminal-loop segment and a different (unknown) region of the genome.

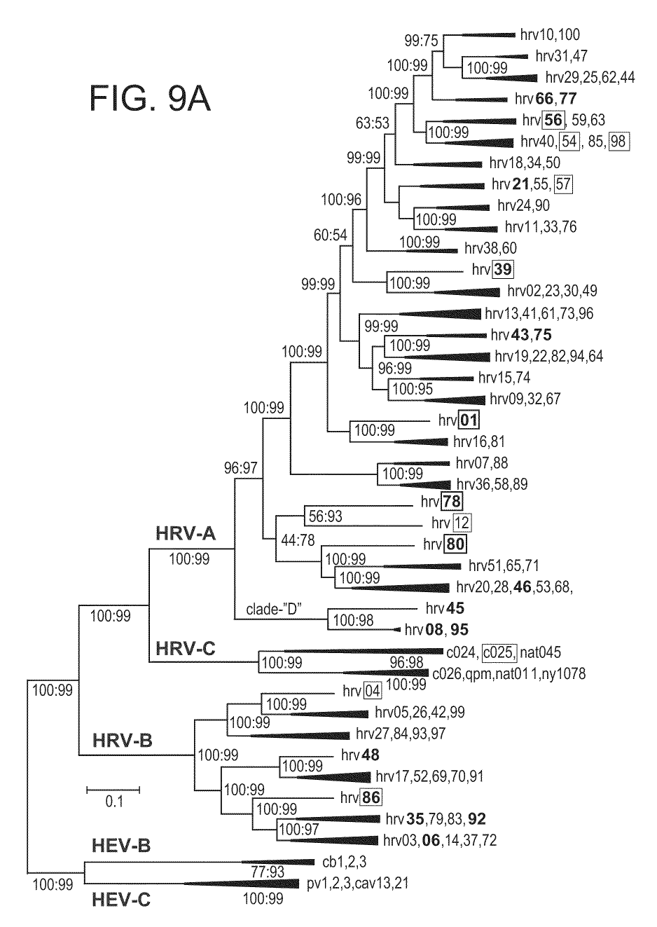

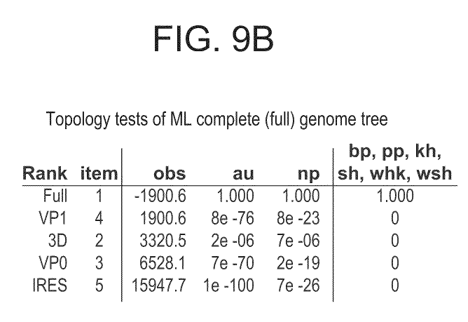

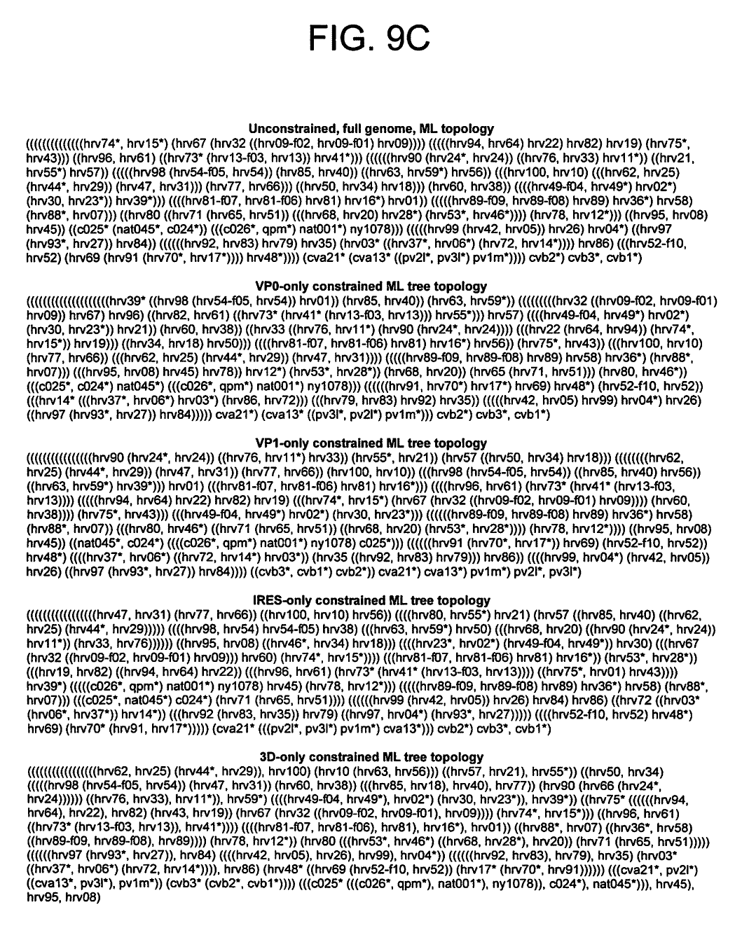

Phylogenetic Relationships of the HRV. Multiple methods were used to compute and compare phylogenetic trees for the aligned RNA and protein data. FIG. 2 is a neighbor-joining consensus tree (K. Tamura, J. Dudley, M. Nei, S. Kumar, Mol. Biol. Evol. 24, 1596 (2007)), which considered the 5′- and 3′-UTRs and the first and second codon positions for the RNA genomes. All major nodes of the tree topology were stable over a range of calculation parameters, regardless of whether they invoked minimum evolution, parsimony, UPGMA, or maximum likelihood (ML) methods (see FIG. 9A for ML tree with bootstrap, likelihood ratio tests, and comparison to the neighbor-joining tree). Statistical comparisons were performed between the ML tree generated from the full genomes, and ML constrained-topology trees generated from capsid sequences that have been used as a surrogate for serotypes (see Methods and FIGS. 9A-C), with the null hypothesis being that the topologies of these limited-sequence trees were the same as the full genome-based tree. The approximately unbiased test calculated from the multiscale bootstrap (au) P-values for the capsid VP1 and VP0 trees were <10⁻⁶⁹, strongly rejecting the null hypothesis. In a similar manner, ML constrained-topology trees generated from IRES, and, 3D-polymerase, sequences were not consistent with the full genome tree (FIGS. 9A, 9B). Additional statistical tests also confirmed these results from the au (FIGS. 9B, 9C) for all four of the constrained-topology trees. Taken together, these results suggest that trees based on sequences from small, albeit biologically important, regions of the HRV genome have a limited capacity to reflect the evolutionary relationships and underlying diversity of HRVs. The tree shown in FIG. 2 also accurately represents parallel calculations on the aligned polyprotein dataset (p-distances differed by <2%). Thus the tree shown in FIG. 2 reveals relationships between the HRVs that have never previously been shown. All aligned full-length sequences contributed to the tree calculations, but published sequences (noted with asterisks on FIG. 2) are shown only as needed, to complete the cohort of reference serotypes (were the redundant published sequences illustrated, without exception they would lie on the same-serotype branches, with p-distances <0.5%). The outer rings of this figure designate major (M: ICAM-1) or minor (m: LDLR) receptor preferences, and also each strain's small-molecule drug reactivity group (“1” or “2”) (K. Andries et al., J. Virol. 64, 1117 (1990)). The tree is shown rooted with three outgroup sequences from the Human Enterovirus C species (poliovirus lm, coxsackieviruses a13 and a21), but of note, the use of five additional outgroups had no effect on overall tree topology or HRV p-distances (FIG. 9). The new field samples (i.e., f01-f10) were assigned tentative serotype names, based on capsid similarity relative to the known reference strains. While some of the full genomes from these samples proved close to their repository cognates, a divergence in sequence for several strains was noted (see below).

According to the tree(s), HRV-A and HRV-C share a common ancestor, which is a sister group to the HRV-B. Although the HRV-C Glade currently has only seven full sequences, its genetic origin is clearly different from the reference set, and the phylogeny indicates that these represent a third HRV species, as has recently been recommended to the International Committee on the Taxonomy of Viruses (on the basis of these data, the International Committee of Taxonomy of Viruses Picornavirus Study Group recently proposed to recognize them as a new species (Nick Knowles, personal communication)). The HRV-C have yet to be cultured or assessed for immunological cross-reactivity, but the sequence space occupied by the available samples suggest that there may be many additional HRV-C strains awaiting discovery. Distance extrapolations relative to the new full reference cohort, predict the HRV-C may have an even broader range of serotypes than the original 99, of which each confers only limited immunologic cross-protection to another.

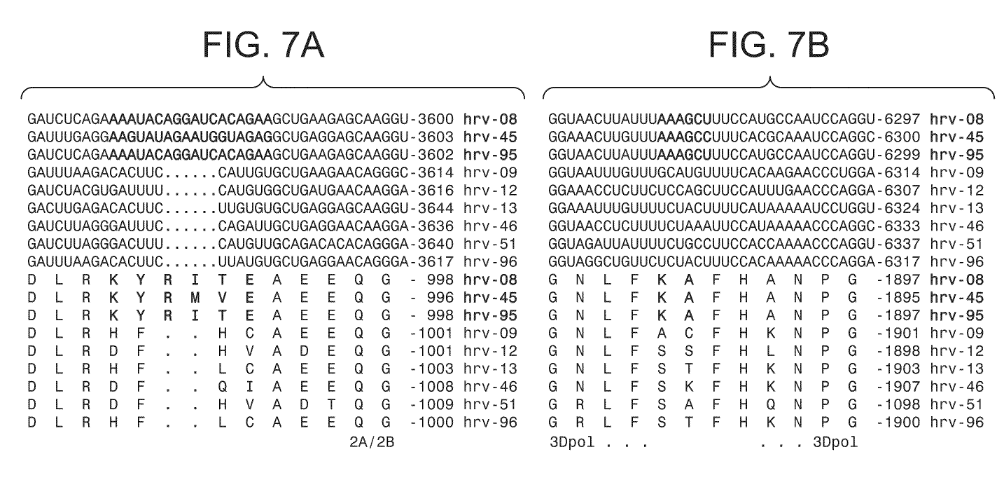

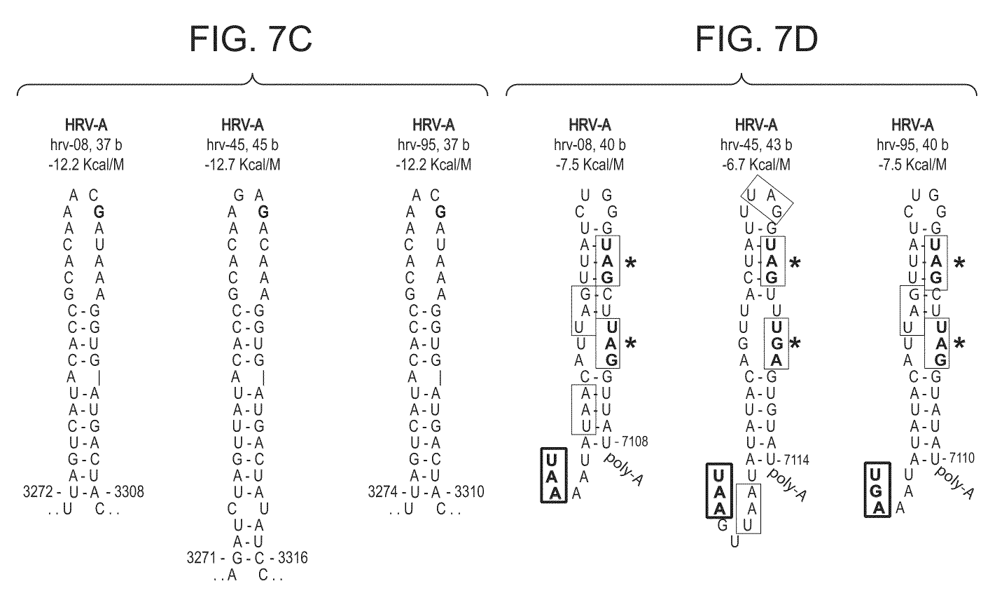

A separate phylogenic finding was the unexpected basal divergence within HRVA of a small (n=3) group of distinct strains, denoted clade-D (hrv-80 and hrv-95 are designated as two different serotypes by ATTC, but differ by ˜150 nucleotides, perhaps indicating a misidentification in the repository)). Although the major basis for discriminating clade-D from other HRV-A lies in their general, genome-length sequence divergence, these particular isolates have RNA elements, such as the cis-acting replication element, the 3′-UTR terminal loop feature (see above and FIG. 6), and local indels and sequence motifs, that are somewhat atypical of other HRV-A strains. Some of the distinguishing characteristics are highlighted in FIG. 7. Among all other major A clades, and major B clades, none have p-distances (>10%) which segregate them so distinctly. Clade D has cautiously been proposed as a fourth species (HRV-D), but the phylogenetic evidence (FIG. 2) and sequence characteristics (FIGS. 6, 7) are highly suggestive. Other early topological divisions within HRV-A separates a major Glade composed of 10 serotypes (counterclockwise, hrv-20 through hrv-12) from a second grouping comprised of ˜12 mini-clades representing 61 serotypes (counterclockwise hrv-89-f09 through hrv-100). These particular relationships were not appreciated when only partial genome sequences (e.g., VP1 or VP4/VP2) were examined (R. M. Ledford et al., J. Virol. 78, 3663 (2004); C. Savolainen, S. Blomqvist, M. N. Mulders, T. Hovi, J. Gen. Virol. 83, 333 (2002)). FIG. 9 shows a comparison of results from trees constructed with whole genomes, VP4/VP2, and VP1 sequence. Neither of the trees derived from the limited sequences revealed the miniclades, clade-D, and multiple other features. Arguably, comparison of full genome data, the context wherein evolutionary events occur, provides the most complete relationship description among the HRVs and allows more accurate historical extrapolations. The phylogenetic diversity described at the whole genome level is consistent with the clinical heterogeneity of HRV infections in humans (J. M. Gwaltney, Jr., J. O. Hendley, G. Simon, W. S. Jordan, Jr., N. Engl. J. Med. 275, 1261 (1966); K. G. Nicholson, J. Kent, D. C. Ireland, BMJ 307, 982 (1993); D. J. Jackson et al., Am. J. Respir. Crit. Care Med. 178, 667 (2008)), although mapping specific clinical characteristics (i.e., incubation period, severity, respiratory compromise, pro-asthmatic phenotypes) to responsible genomic regions will require additional field isolates from a large number of patients with multiple traits. Given the genome-wide diversity that has been documented (5′ spacer elements, ORF start, protease, 3′-UTRs, etc.) clinically relevant relationships may well depend on comparisons from multiple genome regions in addition to the capsids.

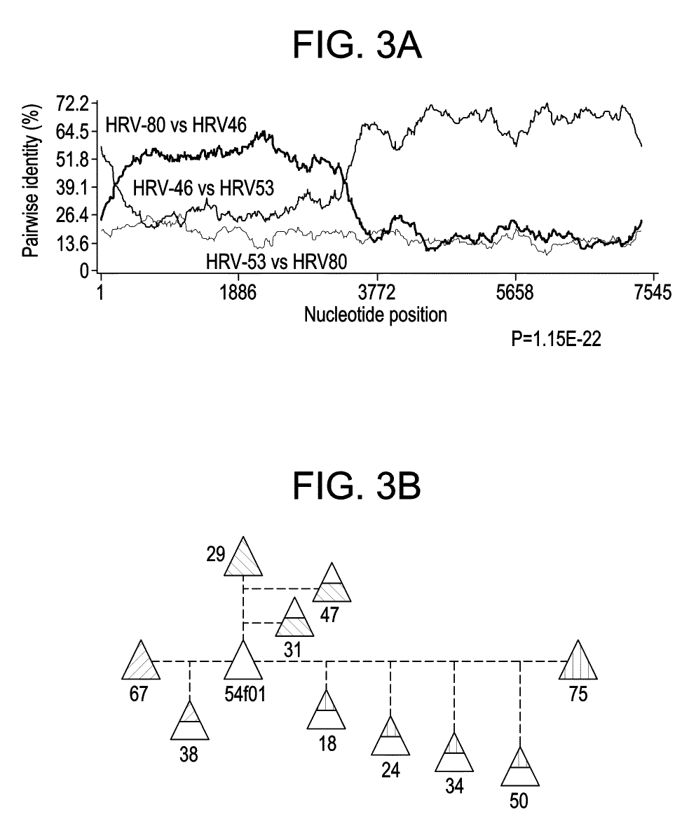

Recombination in HRV. Earlier sequencing reports with a subset of HRV reference genomes concluded that RNA recombination was not a major mechanism for HRV diversity (A. L. Kistler et al., Virol. J. 4, 40 (2007); P. Simmonds, J. Virol. 80, 11124 (2006)), and asserted that known isolates were independently segregating entities. The potential for recombination has been re-evaluated herein by scanning the full reference set and the new field strains with a suite of recombination detection programs (D. Martin, E. Rybicki, Bioinformatics 16, 562 (2000)) relying on phylogenetic distance and sequence similarity. Stringent criteria (probability P value of <0.00001 from 2 or more analyses modes), identified 23 genomes with probable origins resulting from at least twelve independent recombination events. FIG. 3A shows representative data indicating that hrv-46 arose by recombination between hrv-53 (major parent) and hrv-80 (minor parent). Within the hrv-46 genome, nucleotides 32 to 3222 are most similar to hrv-80, while the rest of the genome (nt 3223 to 7200) is common to hrv-53. The result is consistent with this trio's computed phylogenetic relationship (FIG. 2), placing the major parent (hrv-53) and the daughter (hrv-46) in the same Glade, and the minor parent (hrv-80) in a different, nearby Glade. Results for all 23 identified recombination scenarios are summarized in Table 2.

Supportive information and additional statistics are provided in Table 3. Of the recombination locales suggested by these events, the majority (10 of 12) involve the 5′-UTR or the adjacent capsid genes, which seemingly have been collectively rearranged to produce at least 20 separate progeny strains. Among the 138 full-length sequences, hrv-54 (or its ancestor) was apparently the most active in recombination. Field strain hrv-54-f05 links closely to three separate events (see FIG. 3B), contributing to at least seven different serotype progeny. In confirmation studies, a progressive alignment method was used for the HRV genomes (see below) and then the suite of recombination detection programs was repeated. Of the 23 recombination events based on the HMM alignment (Table 2), 19 were also found after the progressive alignment, although in some cases different major and/or minor parents were selected from within the same closely related Glade (see Table 1).

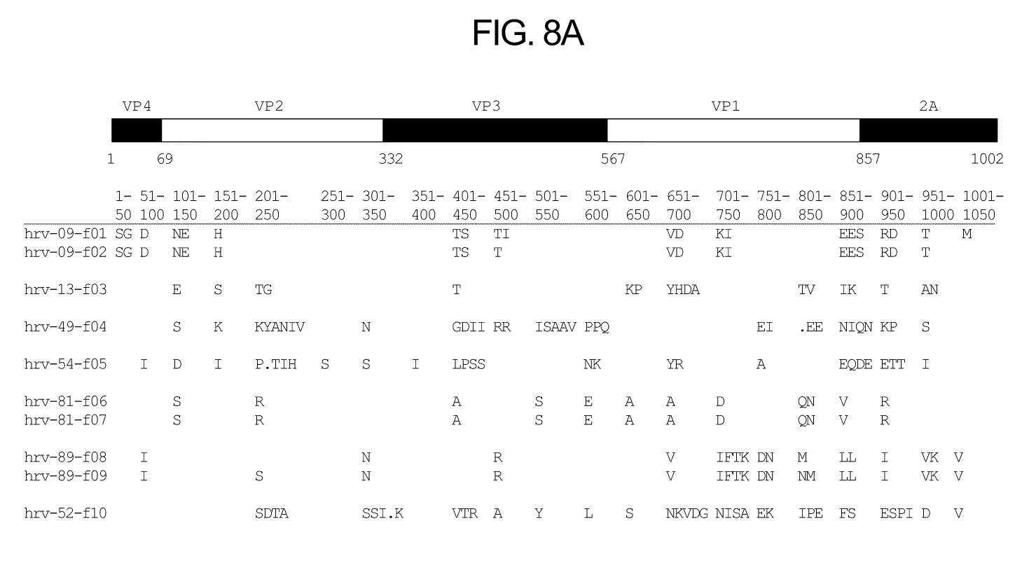

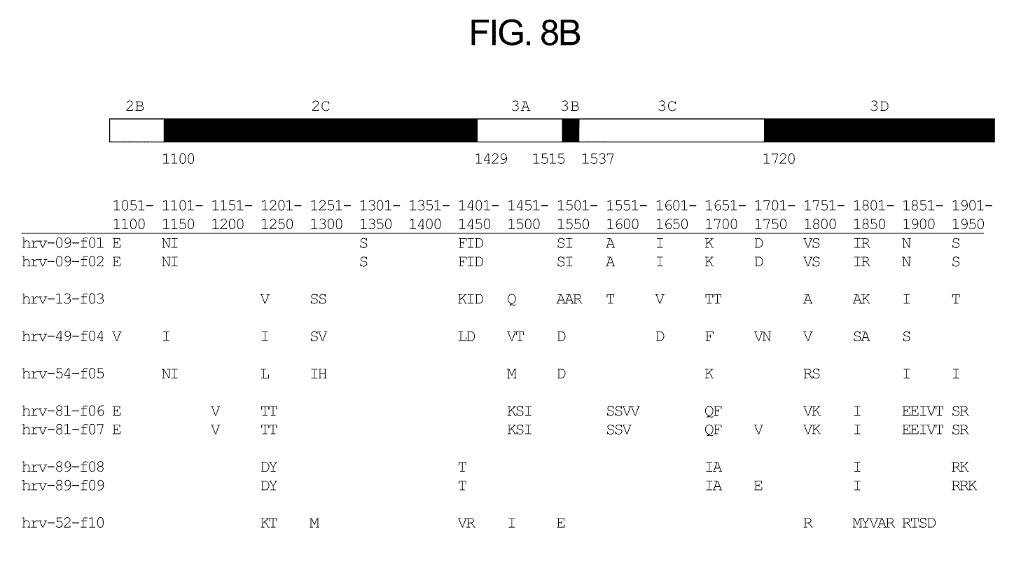

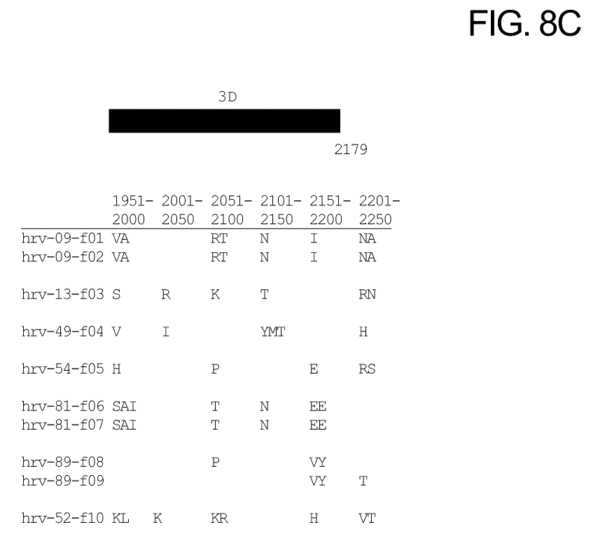

While the sequence fingerprints clearly trace these ancestral patterns, nevertheless all extant progeny also showed evidence of subsequent sequence divergence within the exchanged regions. Recombination was not identified between isolates from different species (i.e., HRV-A and HRV-B), but receptor binding preferences between ICAM and LDLR apparently presented no barriers to exchange. Major group hrv-54 and minor group hrv-29, for example, both contributed to the common ancestor of the minor group viruses, hrv-31 and hrv-47. These results, particularly for HRVs with different receptor preferences, and those from distant clades, such as the parents hrv-21 and hrv-65, suggests that co-infection of the host with multiple parental strains is not uncommon, and may lead to variant progeny with different biological properties. The field isolates also deviated from the reference sequences in a manner that was not confined to any specific portion of the genome. In fact, field strain deviation relative to the reference isolates (for example, hrv-52-f01 vs hrv-52 differ by 838 nucleotides) was frequently greater than that observed between pairs of characterized serotypes (hrv-44 and hrv-29 differ by 385 nucleotides). Indeed, field samples of the same serotype collected from the same geographical region within one year showed marked variability (Table 4 and FIG. 8). The propensity for such variation could underlie the marginal, or lack of efficacy of anti-HRV therapeutics in clinical trials (R. Fleischer, K. Laessig, Clin. Infect. Dis. 37, 1722 (2003); D. L. Barnard, Curr. Pharm. Des. 12, 1379 (2006)). Given the reference set of full genomes, along with the means to rapidly sequence full genomes from field samples, new strategies may become apparent to engineer Glade-specific agents by targeting their commonalities.

In summary, the data complete and define the full set of genome-length sequences in the canonical reference repository of 99 HRV-A and HRV-B serotypes. Alignment and examination of these genomes confirmed species-specific sequence and RNA structural elements which differentiate the HRV-A and HRV-B from newly described HRV-C, and further suggest the HRV-A harbor a distinct, uncharacteristic Glade, which may represent a fourth species (HRV-D). Local sequence variation particularly in the 5′-UTR, characterized each isolate within regions associated with the pathogenic potential for other picornaviruses. Parallel RNA structure comparisons defined several 5′ and 3′ elements as common to all isolates, and unique to the HRV. Motifs like the AUG-presenting 5′ ORF initiation stem, or the UAG-presenting 3′ stem, may contribute to HRV-specific IRES translation mechanisms, ORF termination, or polymerase recognition. It was also found embedded within multiple sequences, including recent field isolates, clear evidence for repeated, historical genome recombination. Co-infection with multiple HRVs is known to occur (C. Savolainen, M. N. Mulders, T. Hovi, Virus Res. 85, 41 (2002)), and it is now known that this can lead to unique strains that may have distinct biologic properties and clinical characteristics. The required host environment for HRV recombination is not known, but with complete genome sequences from additional patient isolates such factors may become apparent. The repository dataset provides a baseline framework for the analysis of additional HRVs that may be in communities, including the HRV-Cs, and will enable larger scale studies of basic molecular and evolutionary characteristics and assignment of disease phenotypes to specific genome regions. Indeed, the recombinations and mutations found in all regions of these genomes suggest that future HRV epidemiologic studies might benefit from full genome sequencing rather than the more limited serotyping. With such an approach, correlations may be more informative in inferring pathogenic potential, and in designing antiviral agents and vaccines.

III. Experiments

A. HRV Samples and Preparation of Viral Nucleic Acids

The HRV reference repository was considered to include all unique HRV serotypes available from American Type Culture Collection (http://www.atcc.org). These amounted to a total of 99 HRVs, as the previously designated “HRV-Hanks” is now considered hrv-21, hrv-87 is now classified as a strain of EV-86, a serotype within the human enterovirus D species, and hrv-1b and hrv-1a are now considered hrv-1. One described HRV serotype (hrv-57) was not available from ATCC and a field sample was utilized whose sequence was consistent with regions of the hrv-57 genome that had been previously reported and this is referred to as a reference sample. Additional field samples were obtained from the Wisconsin State Laboratory of Public Hygiene, collected from 2005-2006. These isolates were amplified in HeLa cells, the virus was concentrated, then snap frozen. Full genome sequencing was performed as described below with 10⁵-10⁶ virions from the contents of one vial of frozen virus provided by the aforementioned sources. Briefly, viral RNA and DNA was prepared in a manner previously described in detail (A. Djikeng et al., BMC Genomics 9, 5 (2008); T. Allander, S. U. Emerson, R. E. Engle, R. H. Purcell, J. Bukh, Proc. Natl. Acad. Sci. U.S.A. 98, 11609 (2001); T. Allander et al., Proc. Natl. Acad. Sci. U.S.A. 102, 12891 (2005)) with minor modifications. Each biological sample was first spun to remove cellular debris and processed through a 0.22 μM filter to enrich viral particles in the flow-through while retaining bacteria and other cells in the filter. To eliminate residual nucleic acid contaminants in the filtrate, 100 units of DNAse I and 3 μg RNAse A were added to the viral resuspension, followed by incubation at 37° C. for 1 hour. RNA was extracted with Trizol-LS reagent (Invitrogen, Carlsbad, Calif.) according to the manufacturer's instructions. The RNA pellet was resuspended in 20 μl of nuclease-free water.

B. Construction of a Library of Random PCR Fragments and Sequencing

The extracted RNA was processed as previously described (1-3). Briefly, 800 ng of purified RNA was reverse-transcribed with SSII Reverse Transcriptase (Invitrogen) using the FR26RV-N primer (5′ GCC GGA GCT CTG CAG ATA TCN NNN NN 3′; SEQ ID NO: 81) at a concentration of 1 μM. In addition, primer FR40RV-T (5′ GCC GGA GCT CTG CAG ATA TC (T)20 3′; SEQ ID NO: 82) was added at a concentration of 5 nM to specifically amplify the polyadenylated 3′ end. The second strand was synthesized by addition of Klenow exo-polymerase (New England Biolabs, Ipswich, Mass.) in the presence of the FR26RV-N random primer. To capture the 5′ end, the Klenow reaction was supplemented with 10-30 nM of primers FR30RVA (5′ GCC GGA GCT CTG CAG ATA TC TTA AAA CTG G 3′; SEQ ID NO: 83) and FR30RVB (5′ GCC GGA GCT CTG CAG ATA TC TTA AAA CAG C 3′; SEQ ID NO: 84) where the final 10 bp match the 5′ ends of A-type and B-type rhinoviruses, respectively.

PCR amplification used high fidelity Taq Gold DNA polymerase (Applied Biosystems, Foster City, Calif.) with the FR20RV primer (5′ GCC GGA GCT CTG CAG ATA TC 3′; SEQ ID NO: 85). PCR amplicons were A-tailed with dATP and 5 units of low fidelity DNA polymerase (Invitrogen) at 72° C. for 30 minutes. A-tailed PCR amplicons were fractionated on a 1% agarose gel and fragments between 500 and 1000 nt were extracted. Amplicons were ligated en masse into the Topo TA cloning vector (Invitrogen) and transformed into competent one-shot Topo top 10 bacteria (Invitrogen). Cells were plated on LB/Amp/XGal agar, and individual colonies were picked for sequencing. The inserted fragments were sequenced bidirectionally with the M13 primers from the Topo TA vector. A total of 192 fragments or more were routinely sequenced per library. Sequencing reactions were performed at the Joint Technology Center, Rockville, Md., on an ABI 3730×1 sequencing system with Big Dye Terminator chemistry (Applied Biosystems).

C. Assembly of Viral Genomes

Sequence reads were downloaded, trimmed to remove primer sequence as well as low quality sequence, and assembled with the program ELVIRA, the Executive for Large-scale Viral Assembly (http://sourceforge.net/projects/elvira). Additional manual inspections identified ambiguities or potential single nucleotide variants and were interrogated by further RT-PCRs, cloning, and sequencing. To close gaps between assembled contigs, strain-specific primers were utilized. Additional primer design, cDNA synthesis and sequencing were performed to ensure at least 4× sequence coverage along all genomes.

D. Polyprotein and RNA Genome Alignments

Founder data for the HRV polyprotein alignment are from superimposition of virion crystal structure hydrogen-bonding maps as described (A. C. Palmenberg, J.-Y. Sgro, in Molecular Biology of Picornaviruses, B. L. Semler, E. Wimmer, Eds. (ASM Press, New York, 2001)). Profile hidden Markov models, derived from the founder data, were progressively augmented with published sequences and those derived from this study. The HMMER program suite (Accelrys, San Diego, Calif.) reported both high and low road fits, building the alignment possibilities with additions from highest to lowest similarity. Each insertion and deletion (indel) in the output iterations was examined and eased within the confines of its high road and low road fits, to maximize conservation of viral cleavage sites, catalytic sites, determined structure landmarks (P1, 2A, 3B-3D) and sequence similarity. The HMM profile of composite HRVs was used to fit three additional HEV-C out-group sequences into the final alignment. Reverse translation of the polyprotein alignment relative to the original RNA sequences formed a core ORF alignment with analogous indels. Preliminary tree-building exercises identified 20 sequences representative of the dominant HRV clades, including three examples from the putative HRV-C species. The complete genome of each select sequence was analyzed separately by mFOLD (A. C. Palmenberg, J.-Y. Sgro, Seminars in Virology 8, 231 (1997)). The consensus topography (optimal plus 100 suboptimal structures) for each the 5′ and 3′ regions of each fold was superimposed into common alignments which maximized analogous base-pair superimposition (5′ cloverleaf, IRES, 3′ stems, etc.) and minimized indels. These foundations were converted into HMM profiles to which the remaining UTR sequences of all HRV and out-groups were fit. In the interest of alignment length, the HEV-C out-group sequences were truncated to remove a 5′ fragment without analog in the HRV (100 b ribosome read-through, 5′ to ORF AUG). The 5′, ORF and 3′ aligned segments for all included sequences were joined contiguously into full-length genome alignments. Again, each indel in the file was re-examined for plausibility, consistency and biological conservation, before the composite alignment was finalized.

E. Phylogenetic Analysis

Phylogenetic analyses on the polyprotein and genome alignments (msf file format) were conducted with MEGA version 4 (K. Tamura, J. Dudley, M. Nei, S. Kumar, Mol. Biol. Evol. 24, 1596 (2007)) and PHYML version 2.4.5 (S. Guindon, O. Gascuel, Syst. Biol. 52, 696 (2003)). HRV sequences from this study (including field strains) were augmented as needed with published data to include at least one representative of the 99 described HRV-A and HRV-B serotypes, and all available (full-length) data from the HRV-C. Multiple tree iterations were evaluated for both the protein (single gene and polyprotein) and RNA genome data, with UPGMA (MEGA), maximum parsimony (MEGA), neighbor joining methods (MEGA) with bootstrap tests (2000×), and maximum likelihood (PHYLM) with approximate likelihood ratio tests (minimum of SH-like and Chi2-based aLRT). None of these methods showed significant topological differences for major branch points with p-values >2 (i.e., 2% change), especially if the 3rd positions of the ORF codons were omitted from consideration in the RNA trees.

In the absence of full-genome data for all reference strains, HRV relationships have been approximated according to more limited sequence sets derived from the VP1, VP0, IRES and 3D regions. To determine whether “serotype” (i.e., VP0 or VP1 sequences) and/or other more conserved regions of the genomes (i.e., IRES or 3D gene) were useful indicators of full strain relationships, defined maximum likelihood (ML) topologies, optimal for these regions were compared statistically with the ML full-genome tree. Optimal ML topologies for the VP1-only (966 b), 3D-only (1389 b), IRES-only (547 b) and VP0-only (86 b VP4+352 b VP2) fragments within the RNA alignment were computed within PhyML, then compared individually against the optimal topology of the full-genome ML tree using PAML and the CONSEL (V0.1i) suite of programs (Shimodaira et al., Bioinformatics 17, 1246 (2001)). The tested (null) hypothesis was the expectation that HRV strains would cluster in reasonable approximations of the full genome data, using only these limited (albeit commonly used) regions of sequence. See also the legends to FIGS. 9A, 9B for specific definitions of the implemented topology tests.

F. 5′- and 3′-Structural Predictions

The accepted notion that thermodynamically derived models for phylogenetically related viruses should exhibit common RNA structural motifs, if such motifs are required for biological activity, was considered. The approach herein has been previously described in detail (A. C. Palmenberg, J.-Y. Sgro, Seminars in Virology 8, 231 (1997)). Briefly, rather than form structure predictions on the basis of sequence similarities, folding was undertaken first, and then a searched was conducted among the most probable configurations (energy minimization) for regions with consistent structures. As described above for the RNA alignments, full genome sequences for 20-40 hrv, representing different phylogenetic clades, were evaluated in their entirety by mFOLD (A. C. Palmenberg, J.-Y. Sgro, Seminars in Virology 8, 231 (1997)) asking for the optimal, and up to 100 closely related (+12 Kcal) suboptimal configurations. Without exception, the consensus fold for each sequence (required >80% of queried connect files) agreed that the 5′ and 3′ ends of each RNA generally configured independently. That is, few if any segments within these regions made preferred (low energy) long-range contacts with interior portions of the genome. The UTR topologies folded regionally as a series of local, connected motifs. This tendency was confirmed by assessing the P-num values, computed for the whole-genome folds. The pairing number (Pnum) is a quantitative measure of the propensity of any given base to become involved with the same or alternative pairing partners in a collection of suboptimal folds. It has been shown for other viral sequences that low P-num bases and their correlate partners usually dominate the most important helices and stems supporting biologically significant motifs, especially within the lowest energy configurations. These bases and their partners were therefore used to identify and align true homologues (functional analogues) at the primary sequence level, even in regions with less-than obvious conservation. Superimposition of the 5′ and 3′ low P-num motifs from the genome folds formed the core consensus profiles for the RNA alignments in these regions (as described above) and also identified multiple specific conserved motifs throughout the genomes. Once these commonalities were identified for the 5′ cloverleaf, IRES, 5′ ORF initiation stem, cre element, and 3′-UTR, all other sequences in the alignment were re-folded in these regions (mFold, with 50 suboptimals), to confirm that they too had similar, conserved, low energy, low P-num motifs.

G. Recombination Analysis

The recombination predictions of the genomic sequences, aligned as described above, were conducted with a suite of programs within the RDP3 package (D. P. Martin, C. Williamson, D. Posada, Bioinformatics 21, 260 (2005)). The individual programs RDP (D. Martin, E. Rybicki, Bioinformatics 16, 562 (2000)), Bootscan (M. O, Salminen, J. K. Carr, D. S. Burke, F. E. McCutchan, AIDS Res. Hum. Retroviruses 11, 1423 (1995)), Maximum X2 (J. M. Smith, J. Mol. Evol. 34, 126 (1992)), Chimaera (D. P. Martin, C. Williamson, D. Posada, Bioinformatics 21, 260 (2005)), SiScan (M. J. Gibbs, J. S. Armstrong, A. J. Gibbs, Bioinformatics 16, 573 (2000)) and 3Seq (M. F. Boni, D. Posada, M. W. Feldman, Genetics 176, 1035 (2007)), were implemented for the analysis. Since no single program provides optimal performance under all conditions, any event supported by evidence from two or more analyses with P-values <0.00001 was considered a result consistent with recombination. Potential recombination events were also assessed by phylogenetic analysis, breakpoint polishing and alignment consistency checks. For each individual program, default settings were used except as specified: RDP, internal reference only, window size for recombination, 100 by with step size 10 bp; GENECONV, G-scale was set as 3; BootScan, number of bootstrap replicates, 200, window size, 100 bp, step size, 10 bp, model options: Jukes and Cantor, 1969; Maxichi, variable window size was used, strip gap was selected; SiScan, window size, 100 by with step size 10 bp, P-value permutation number was set as 1000. In additional analyses, to confirm the recombinations that were found, genomic sequences were aligned using progressive alignment methods (C. Notredame, D. G. Higgins, J. Heringa, J. Mol. Biol. 302, 205 (2000)). Briefly, the progressive alignments were performed with non-coding (R. C. Edgar, Nucleic Acids Res. 32, 1792 (2004)) and coding regions (C. Notredame, D. G. Higgins, J. Heringa, J. Mol. Biol. 302, 205 (2000); M. Suyama, D. Torrents, P. Bork, Nucleic Acids Res. 34, W609-W612 (2006)) using the referenced programs and the alignments were concatenated by a custom script. The subsequent final alignment was utilized for recombination predictions using the RDP3 package as described above.

IV. Determining HRV Serotype in a Patient Sample

A sample from a patient, e.g., a sample from the respiratory tract (e.g., nose, mouth, larynx, trachea, bronchi, or alveoli), eyes, ears, blood, urine or stool, or from a culture derived from these samples, is sequenced, e.g., using a sequence-independent random priming method well-known in the art (A. Djikeng et al., BMC Genomics 9, 5 (2008); A. C. Palmenberg et al., Science 324:55 (2009).

Briefly, the sample is passed through a 0.22 micron filter and the filtrate is then treated with RNase A and DNase 1 at 37° C. for one hour (if the preparation consists of purified HRV this step can be eliminated). The RNA is then extracted by phenol chloroform extraction using methods well-established in the field. Samples are treated with Trizol-LS (Invitrogen, Carlsbad, Calif.) for 5 minutes at room temperature and then chloroform, and the aqueous phase is transferred to a new tube to which isopropanol is added and incubated for 10 minutes at 25° C. and centrifuged for 5 minutes at 4° C. The supernatant is discarded and the pellet is treated with 70% ethanol at 4° C. and centrifuged for 5 minutes at 4° C. The RNA-rich supernatant is removed and the pellet is air dried for 3-5 minutes and resuspended in RNase free water. This can be stored at ˜80° C. or the next step undertaken.

From the RNA sample ˜800 ng is subjected to reverse transcription to produce DNA by the use of SS11 Reverse Transcriptase (Invitrogen) and multiple primers that incorporate random nucleotide sequences. One example is the primer 5′ GCC GGA GCT CAG ATA TCN NNN NN 3′ (SEQ ID NO: 86) where N is either AGC or T. Ideally, then, the reaction contains 1296 primers (6⁴) since this represents all possible combinations. Other primers could consist of ˜20-mers of all possible combinations. Also, the number of primers can be less than the theoretical maximal number of combinations. The second strand of DNA can be synthesized using Klenow exo-polymerase (New England Biolabs, Ipswich, Mass.) and the same set of primers to produce double-stranded DNA. However, the RNA:DNA complex formed from the reverse transcriptase can be used directly for the next step without the Klenow fill-in. To capture the 5′ end, the Klenow reaction can be supplemented with 10-30 nM of the primers: 5′ GCC GGA GCT CTG CAG ATA TC TTA AAA CTG G 3′ (SEQ ID NO: 83) or 5′ GCC GGA GCT CTG CAG ATA TC TTA AAA CAG C 3′ (SEQ ID NO: 84) where the final 10 bp universally match the 5′ ends of HRVs. The double-stranded DNA or the RNA:DNA complex is then subjected to a polymerase chain reaction using high-fidelity Taq Gold DNA polymerase (Applied Biosystems, Foster City, Calif.) using a primer such as 5′ GCC GGA GCT CTG CAG ATA TC 3′ (SEQ ID NO: 85). PCR amplicons are A-tailed with dATP and 5 units of low fidelity DNA polymerase (Invitrogen) at 72° C. for 30 minutes. A-tailed PCR amplicons are fractionated on a 1% agarose gel and fragments between 500 and 1000 nt are extracted. Amplicons are ligated en masse into the Topo TA cloning vector (Invitrogen) and transformed into competent one-shot Topo top 10 bacteria (Invitrogen). Cells are plated on LB/Amp/XGal agar, and individual colonies are picked, grown, and DNA prepared. The inserted fragments of HRV DNA are sequenced bidirectionally using the M13 primers which are incorporated in the Topo TA vector. One hundred and ninety-two clones or more, if needed, are then sequenced on an Applied Biosystems 3730×1 sequencing system with BigDye Terminator chemistry (Applied Biosystems). Sequence reads are downloaded, trimmed to remove primer sequence as well as low quality sequence, and can be assembled with the program ELVIRA (http://sourceforge.net/projects/elvira; see, e.g., Palmenberg et al., Science 324:55 (2009), or other widely available similar programs. To close gaps between assembled contigs, specific primers can be designed from the existing sequence and the polymerase chain reaction performed. The described procedure results in about four-fold redundancy in the sequence coverage of the entire HRV genome.

To ascertain the specific serotype (or strain) of the HRV isolated from a patient sample, the completed full genome sequence is compared to the 80 full-length novel genome sequences described herein, and optionally the additional 26 that are known from the literature. Although less than full-length sequences can be used for serotype identification, use of shorter sequences (i.e., less than the full genome) will likely result in a higher number of incomplete serotype identifications, because we have ascertained, from patient samples, that HRV recombines in vivo. The software used for comparing HRV sequences from a sample must be able to accommodate all the reference HRVs and the patient samples, and to identify the match using stringent criteria. There are multiple programs that accomplish this, including MUSCLE (http://www.ebi.ac.uk/Tools/muscle/index.html; Edgar, BMC Bioinformatics 5:113 (2004)), MacVector (Accelrys, San Diego, Calif.), and ClustalW (http://www.ebi.ac.uk/Tools/clustalw2/; Thompson et al., Nucleic Acids Res. 22:4673 (1994)).

While the foregoing specification teaches the principles of the present invention, with examples provided for the purpose of illustration, it will be appreciated by one skilled in the art from reading this disclosure that various changes in form and detail can be made without departing from the true scope of the invention.

All materials referred to herein, including books, manuals, journal publications, posters, abstracts, talks, patents, GenBank records and submissions, and published patent applications, are incorporated herein in their entireties.

TABLE 1
Guide to Sequences and Strains

| msf | | | | name in | | | |
|---|---|---|---|---|---|---|---|
| file | | sero- | | alignment and | name on | accession # | |
| order (1) | species (2) | type (3) | strain (4) | SEQ ID NO. (5) | tree (6) | or seq origin (7) | receptor (8) |
| — | — | — | — | hrv-ann(18) | — | — | — |
| 1 | HEV-C | a13 | Flores | af499637-cva13 | cva-13* | AF499637 | ICAM1 |
| 2 | HEV-C | a21 | Kuykendall | af546702-cva21 | cva-21* | AF546702 | ICAM1 |
| 3 | HEV-C | pv1 | Mahoney | v01149-pv1m | pv-1m* | V01149 | PVR |
| 4 | HRV-A | 1 | b, B632 | d00239-01 | not inc | D00239 | LDLR |
| 5 | HRV-A | 1 | a, 20601- | pico-01 | not inc | Pico-DB | LDLR |
| | | | Ohio | | | | |
| 6** | HRV-A | 1 | A | hrv-01 | hrv-01 | FJ445111 | LDLR |
| | | | | SEQ ID NO: 1 | | | |
| 7 | HRV-A | 2 | HGP | x02316-02 | hrv-02* | X02316 | LDLR |
| 8** | HRV-A | 7 | ATCC | hrv-07 | hrv-07 | FJ445176 | ICAM1 |
| | | | | SEQ ID NO: 3 | | | |
| 9 | HRV-A | 7 | 68-CV11 | dq473503-07 | not inc | DQ473503 | ICAM1 |
| 10** | HRV-A | 8 | ATCC | hrv-08 | hrv-08 | FJ445113 | ICAM1 |
| | | | | SEQ ID NO: 4 | | | |
| 11 | HRV-A | 9 | 211-CV13 | pico-09 | not inc | Pico-DB | ICAM1 |
| 12** | HRV-A | 9 | ATCC | hrv-09 | hrv-09 | FJ445177 | ICAM1 |
| | | | | SEQ ID NO: 7 | | | |
| 13** | HRV-A | 9 | fs ship#2, | hrv-09-f01 | hrv-09-f01 | FJ445114 | ICAM1 |
| | | | isolate#A | SEQ ID NO: 5 | | | |
| 14** | HRV-A | 9 | fs ship#2, | hrv-09-f02 | hrv-09-f02 | FJ445115 | ICAM1 |
| | | | isolate#B | SEQ ID NO: 6 | | | |
| 15 | HRV-A | 10 | 204-CV14 | dq473498-10 | not inc | DQ473498 | ICAM1 |
| 16** | HRV-A | 10 | ATCC | hrv-10 | hrv-10 | FJ445178 | ICAM1 |
| | | | | SEQ ID NO: 8 | | | |
| 17 | HRV-A | 11 | 1-CV15 | ef173414-11 | hrv-11* | EF173414 | ICAM1 |
| 18 | HRV-A | 12 | 181-CV16 | ef173415-12 | hrv-12* | EF173415 | ICAM1 |
| 19** | HRV-A | 13 | ATCC | hrv-13 | hrv-13 | FJ445116 | ICAM1 |
| | | | | SEQ ID NO: 10 | | | |
| 20** | HRV-A | 13 | fs ship#1 | hrv-13-f03 | hrv-13-f03 | FJ445117 | ICAM1 |
| | | | | SEQ ID NO: 9 | | | |
| 21 | HRV-A | 15 | 1734-South | dq473493-15 | hrv-15* | DQ473493 | ICAM1 |
| | | | Carolina/60 | | | | |
| 22 | HRV-A | 16 | 11757- | l24917-16 | hry-16* | L24917 | ICAM1 |
| | | | Washington | | | | |
| | | | DC/60 | | | | |
| 23** | HRV-A | 18 | ATCC | hrv-18 | hrv-18 | FJ445118 | ICAM1 |
| | | | | SEQ ID NO: 11 | | | |
| 24** | HRV-A | 19 | ATCC | hrv-19 | hrv-19 | FJ445119 | ICAM1 |
| | | | | SEQ ID NO: 12 | | | |
| 25** | HRV-A | 20 | ATCC | hrv-20 | hrv-20 | FJ445120 | ICAM1 |
| | | | | SEQ ID NO: 13 | | | |
| 26** | HRV-A | 21 | ATCC | hrv-21 | hrv-21 | FJ445121 | ICAM1 |
| | | | | SEQ ID NO: 14 | | | |
| 27** | HRV-A | 22 | ATCC | hrv-22 | hrv-22 | FJ445122 | ICAM1 |
| | | | | SEQ ID NO: 15 | | | |
| 28 | HRV-A | 23 | 5124-CV24 | dq473497-23 | hrv-23* | DQ473497 | ICAM1 |
| 29** | HRV-A | 24 | ATCC | hrv-24 | hrv-24 | FJ445190 | ICAM1 |
| | | | | SEQ ID NO: 16 | | | |
| 30 | HRV-A | 24 | 5146-CV25 | ef173416-24 | not inc | EF173416 | ICAM1 |
| 31** | HRV-A | 25 | ATCC | hrv-25 | hrv-25 | FJ445123 | ICAM1 |
| | | | | SEQ ID NO: 17 | | | |
| 32 | HRV-A | 28 | 6101-CV29 | dq473508-28 | hrv-28* | DQ473508 | ICAM1 |
| 33** | HRV-A | 29 | ATCC | hrv-29 | hrv-29 | FJ445125 | LDLR |
| | | | | SEQ ID NO: 20 | | | |
| 34 | HRV-A | 30 | 106F | dq473512-30 | not inc | DQ473512 | LDLR |
| 35** | HRV-A | 30 | ATCC | hrv-30 | hrv-30 | FJ445179 | LDLR |
| | | | | SEQ ID NO: 21 | | | |
| 36** | HRV-A | 31 | ATCC | hrv-31 | hrv-31 | FJ445126 | LDLR |
| | | | | SEQ ID NO: 22 | | | |
| 37** | HRV-A | 32 | ATCC | hrv-32 | hrv-32 | FJ445127 | ICAM1 |
| | | | | SEQ ID NO: 23 | | | |
| 38** | HRV-A | 33 | ATCC | hrv-33 | hrv-33 | FJ445128 | ICAM1 |
| | | | | SEQ ID NO: 24 | | | |
| 39** | HRV-A | 34 | ATCC | hrv-34 | hrv-34 | FJ445189 | ICAM1 |
| | | | | SEQ ID NO: 25 | | | |
| 40 | HRV-A | 34 | 137-3 | dq473501-34 | not inc | DQ473501 | ICAM1 |
| 41 | HRV-A | 36 | 342H | dq473505-36 | hrv-36* | DQ473505 | ICAM1 |
| 42** | HRV-A | 38 | ATCC | hrv-38 | hrv-38 | FJ445180 | ICAM1 |
| | | | | SEQ ID NO: 27 | | | |
| 43 | HRV-A | 38 | CH79 | dq473495-38 | not inc | DQ473495 | ICAM1 |
| 44 | HRV-A | 39 | 209- | ay751783-39 | hrv-39* | AY751783 | ICAM1 |
| | | | Maryland/62 | | | | |
| 45** | HRV-A | 40 | ATCC | hrv-40 | hrv-40 | FJ445129 | ICAM1 |
| | | | | SEQ ID NO: 28 | | | |
| 46 | HRV-A | 41 | 56110-North | dq473491-41 | hrv-41* | DQ473491 | ICAM1 |
| | | | Carolina/61 | | | | |
| 47** | HRV-A | 43 | ATCC | hrv-43 | hrv-43 | FJ445131 | ICAM1 |
| | | | | SEQ ID NO: 30 | | | |
| 48 | HRV-A | 44 | 71560-North | dq473499-44 | hrv-44* | DQ473499 | LDLR |
| | | | Carolina/61 | | | | |
| 49** | HRV-A | 45 | ATCC | hrv-45 | hrv-45 | FJ445132 | ICAM1 |
| | | | | SEQ ID NO: 31 | | | |
| 50 | HRV-A | 46 | Crell-Baylor | dq473506-46 | hrv-46* | DQ473506 | ICAM1 |
| | | | 2-Texas/64 | | | | |
| 51** | HRV-A | 47 | ATCC | hrv-47 | hrv-47 | FJ445133 | LDLR |
| | | | | SEQ ID NO: 32 | | | |
| 52 | HRV-A | 49 | 8213 | dq473496-49 | hrv-49* | DQ473496 | LDLR |
| 53** | HRV-A | 49 | fs ship#2 | hrv-49-f04 | hrv-49-f04 | FJ445134 | LDLR |
| | | | | SEQ ID NO: 33 | | | |
| 54** | HRV-A | 50 | ATCC | hrv-50 | hrv-50 | FJ445135 | ICAM1 |
| | | | | SEQ ID NO: 34 | | | |
| 55** | HRV-A | 51 | ATCC | hrv-51 | hrv-51 | FJ445136 | ICAM1 |
| | | | | SEQ ID NO: 35 | | | |
| 56 | HRV-A | 53 | F01-3928 | dq473507-53 | hrv-53* | DQ473507 | ICAM1 |
| 57** | HRV-A | 54 | ATCC | hrv-54 | hrv-54 | FJ445138 | ICAM1 |
| | | | | SEQ ID NO: 39 | | | |
| 58** | HRV-A | 54 | fs ship#1 | hry-54-f05 | hrv-54-f05 | FJ445139 | ICAM1 |
| | | | | SEQ ID NO: 38 | | | |
| 59 | HRV-A | 55 | Wis315E- | dq473511-55 | hrv-55* | DQ473511 | ICAM1 |
| | | | Wisconsin-64 | | | | |
| 60** | HRV-A | 56 | ATCC | hrv-56 | hrv-56 | FJ445140 | ICAM1 |
| | | | | SEQ ID NO: 40 | | | |
| 61** | HRV-A | 57 | fs ship#1 | hrv-57 | hrv-57 | FJ445141 | ICAM1 |
| | | | | SEQ ID NO: 41 | | | |
| 62** | HRV-A | 58 | ATCC | hrv-58 | hrv-58 | FJ445142 | ICAM1 |
| | | | | SEQ ID NO: 42 | | | |
| 63 | HRV-A | 59 | 611-CV35 | dq473500-59 | hrv-59* | DQ473500 | ICAM1 |
| 64** | HRV-A | 60 | ATCC | hrv-60 | hrv-60 | FJ445143 | LDLR |
| | | | | SEQ ID NO: 43 | | | |
| 65** | HRV-A | 61 | ATCC | hrv-61 | hrv-61 | FJ445144 | ICAM1 |
| | | | | SEQ ID NO: 44 | | | |
| 66** | HRV-A | 62 | ATCC | hrv-62 | hrv-62 | FJ445145 | LDLR |
| | | | | SEQ ID NO: 45 | | | |
| 67** | HRV-A | 63 | ATCC | hrv-63 | hrv-63 | FJ445146 | ICAM1 |
| | | | | SEQ ID NO: 46 | | | |
| 68** | HRV-A | 64 | ATCC | hrv-64 | hrv-64 | FJ445181 | ICAM1 |
| | | | | SEQ ID NO: 47 | | | |
| 69 | HRV-A | 64 | 6258-CV44 | ef173417-64 | not inc | EF173417 | ICAM1 |
| 70** | HRV-A | 65 | ATCC | hrv-65 | hrv-65 | FJ445147 | ICAM1 |
| | | | | SEQ ID NO: 48 | | | |
| 71** | HRV-A | 66 | ATCC | hrv-66 | hrv-66 | FJ445148 | ICAM1 |
| | | | | SEQ ID NO: 49 | | | |
| 72** | HRV-A | 67 | ATCC | hrv-67 | hrv-67 | FJ445149 | ICAM1 |
| | | | | SEQ ID NO: 50 | | | |
| 73** | HRV-A | 68 | ATCC | hrv-68 | hrv-68 | FJ445150 | ICAM1 |
| | | | | SEQ ID NO: 51 | | | |
| 74** | HRV-A | 71 | ATCC | hrv-71 | hrv-71 | FJ445152 | ICAM1 |
| | | | | SEQ ID NO: 53 | | | |
| 75 | HRV-A | 73 | 107E | dq473492-73 | hrv-73* | DQ473492 | ICAM1 |
| 76 | HRV-A | 74 | 328A | dq473494-74 | hrv-74* | DQ473494 | ICAM1 |
| 77 | HRV-A | 75 | 328F | dq473510-75 | hrv-75* | DQ473510 | ICAM1 |
| 78 | HRV-A | 76 | H00062 | dq473502-76 | not inc | DQ473502 | ICAM1 |
| 79** | HRV-A | 76 | ATCC 1x | hrv-76 | hrv-76 | FJ445182 | ICAM1 |
| | | | 3185-3310 | SEQ ID NO: 55 | | | |
| 80** | HRV-A | 77 | ATCC | hrv-77 | hrv-77 | FJ445154 | ICAM1 |
| | | | | SEQ ID NO: 56 | | | |
| 81** | HRV-A | 78 | ATCC | hrv-78 | hrv-78 | FJ445183 | ICAM1 |
| | | | | SEQ ID NO: 57 | | | |
| 82 | HRV-A | 78 | 2030-65 | ef173418-78 | not inc | EF173418 | ICAM1 |
| 83** | HRV-A | 80 | ATCC | hrv-80 | hrv-80 | FJ445156 | ICAM1 |
| | | | | SEQ ID NO: 59 | | | |
| 84** | HRV-A | 81 | ATCC | hrv-81 | hrv-81 | FJ445157 | ICAM1 |
| | | | | SEQ ID NO: 62 | | | |
| 85** | HRV-A | 81 | fs ship#1 | hrv-81-f06 | hrv-81-f06 | FJ445158 | ICAM1 |
| | | | | SEQ ID NO: 60 | | | |
| 86** | HRV-A | 81 | fs ship#2 | hrv-81-f07 | hrv-81-f07 | FJ445159 | ICAM1 |
| | | | | SEQ ID NO: 61 | | | |
| 87** | HRV-A | 82 | ATCC | hrv-82 | hrv-82 | FJ445160 | ICAM1 |
| | | | | SEQ ID NO: 63 | | | |
| 88 | HRV-A | 82 | Santa Cruz, | dq473509-82 | not inc | DQ473509 | ICAM1 |
| | | | CA | | | | |
| 89** | HRV-A | 85 | ATCC | hrv-85 | hrv-85 | FJ445163 | ICAM1 |
| | | | | SEQ ID NO: 66 | | | |
| 90 | HRV-A | 85 | 50-525-CV54 | pico-85 | not inc | Pico-DB | ICAM1 |
| 91 | HRV-A | 88 | CVD 01- | dq473504-88 | hrv-88* | DQ473504 | ICAM1 |
| | | | 0165-Dam- | | | | |
| | | | brauskas | | | | |
| 92 | HRV-A | 89 | na | a10937-89 | not inc | A10937 | ICAM1 |
| 93** | HRV-A | 89 | ATCC | hrv-89 | hrv-89 | FJ445184 | ICAM1 |
| | | | | SEQ ID NO: 70 | | | |
| 94** | HRV-A | 89 | fs ship#2 | hrv-89-f08 | hrv-89-f08 | FJ445166 | ICAM1 |
| | | | isolate#A | SEQ ID NO: 68 | | | |
| 95** | HRV-A | 89 | fs ship#2 | hrv-89-f09 | hrv-89-f09 | FJ445165 | ICAM1 |
| | | | isolate#B | SEQ ID NO: 69 | | | |
| 96 | HRV-A | 89 | 41467-Gallo | m16248-89 | not inc | M16248 | ICAM1 |
| 97** | HRV-A | 90 | ATCC | hrv-90 | hrv-90 | FJ445167 | ICAM1 |
| | | | | SEQ ID NO: 71 | | | |
| 98** | HRV-A | 94 | ATCC | hrv-94 | hrv-94 | FJ445185 | ICAM1 |
| | | | | SEQ ID NO: 74 | | | |
| 99 | HRV-A | 94 | SF-1803 | ef173419-94 | not inc | EF173419 | ICAM1 |
| 100** | HRV-A | 95 | ATCC | hrv-95 | hrv-95 | FJ445170 | ICAM1 |
| | | | | SEQ ID NO: 75 | | | |
| 101** | HRV-A | 96 | ATCC | hrv-96 | hrv-96 | FJ445171 | ICAM1 |
| | | | | SEQ ID NO: 76 | | | |
| 102** | HRV-A | 98 | ATCC | hrv-98 | hrv-98 | FJ445173 | ICAM1 |
| | | | | SEQ ID NO: 78 | | | |
| 103** | HRV-A | 100 | ATCC | hrv-100 | hrv-100 | FJ445175 | ICAM1 |
| | | | | SEQ ID NO: 80 | | | |
| 104 | HRV-C | na | QPM | ef186077-qpm | qpm* | EF186077 | ICAM1 |
| 105 | HRV-C | na | Nat001 | ef077279-nat001 | nat001* | EF077279(16) | ICAM1 |
| 106 | HRV-C | na | ny1078 | ny1078 | ny1078 | unpub(17) | ICAM1 |
| 107 | HRV-C | na | c024 | ef582385-c024 | c024* | EF582385 | ICAM1 |
| 108 | HRV-C | na | Nat045 | ef077280-nat045 | nat045* | EF077280 | ICAM1 |
| 109 | HRV-C | na | c026 | ef582387-c026 | c026* | EF582387 | ICAM1 |
| 110 | HRV-C | na | c025 | ef582386-c025 | c025* | EF582386 | ICAM1 |
| 111 | HRV-B | 3 | FEB | dq473485-03 | hrv-03* | DQ473485 | ICAM1 |
| 112 | HRV-B | 3 | FEB | ef173422-03 | not inc | EF173422 | ICAM1 |
| 113 | HRV-B | 4 | 16/60 | dq473490-04 | hrv-04* | DQ473490 | ICAM1 |
| 114** | HRV-B | 5 | ATCC | hrv-05 | hrv-05 | FJ445112 | ICAM1 |
| | | | | SEQ ID NO: 2 | | | |
| 115 | HRV-B | 6 | Thompson | dq473486-06 | hrv-06* | DQ473486 | ICAM1 |
| 116 | HRV-B | 14 | 1059-South | l05355-14 | hrv-14* | L05355 | ICAM1 |
| | | | Carolina/59 | | | | |
| 117 | HRV-B | 14 | 1060-South | k02121-14 | not inc | K02121 | ICAM1 |
| | | | Carolina/59 | | | | |
| 118 | HRV-B | 17 | 33342-North | ef173420-17 | hrv-17* | EF173420 | ICAM1 |
| | | | Carolina/59 | | | | |
| 119** | HRV-B | 26 | ATCC | hrv-26 | hrv-26 | FJ445124 | ICAM1 |
| | | | | SEQ ID NO: 18 | | | |
| 120** | HRV-B | 27 | ATCC | hrv-27 | hrv-27 | FJ445186 | ICAM1 |
| | | | | SEQ ID NO: 19 | | | |
| 121 | HRV-B | 27 | 5870-CV28 | ef173421-27 | not inc | EF173421 | ICAM1 |
| 122 | HRV-B | 35 | 164A | dq473487-35 | not inc | DQ473487 | ICAM1 |
| 123** | HRV-B | 35 | ATCC | hrv-35 | hrv-35 | FJ445187 | ICAM1 |
| | | | | SEQ ID NO: 26 | | | |
| 124 | HRV-B | 37 | 151-1 | ef173423-37 | hrv-37* | EF173423 | ICAM1 |
| 125** | HRV-B | 42 | ATCC | hrv-42 | hrv-42 | FJ445130 | ICAM1 |
| | | | | SEQ ID NO: 29 | | | |
| 126 | HRV-B | 48 | 1505 | dq473488-48 | hrv-48* | DQ473488 | ICAM1 |
| 127** | HRV-B | 52 | ATCC | hrv-52 | hrv-52 | FJ445188 | ICAM1 |
| | | | | SEQ ID NO: 37 | | | |
| 128** | HRV-B | 52 | fs ship#1 | hrv-52-f10 | hrv-52-f10 | FJ445137 | ICAM1 |
| | | | | SEQ ID NO: 36 | | | |
| 129 | HRV-B | 52 | F01-3772 | ef173424-52 | not inc | EF173424 | ICAM1 |
| 130** | HRV-B | 69 | ATCC | hrv-69 | hrv-69 | FJ445151 | ICAM1 |
| | | | | SEQ ID NO: 52 | | | |
| 131 | HRV-B | 70 | F02-2547- | dq473489-70 | hrv-70* | DQ473489 | ICAM1 |
| | | | Treganza | | | | |
| 132** | HRV-B | 72 | ATCC | hrv-72 | hrv-72 | FJ445153 | ICAM1 |
| | | | | SEQ ID NO: 54 | | | |
| 133** | HRV-B | 79 | ATCC | hrv-79 | hrv-79 | FJ445155 | ICAM1 |
| | | | | SEQ ID NO: 58 | | | |
| 134** | HRV-B | 83 | ATCC | hrv-83 | hrv-83 | FJ445161 | ICAM1 |
| | | | | SEQ ID NO: 64 | | | |
| 135** | HRV-B | 84 | ATCC | hrv-84 | hrv-84 | FJ445162 | ICAM1 |
| | | | | SEQ ID NO: 65 | | | |
| 136** | HRV-B | 86 | ATCC | hrv-86 | hrv-86 | FJ445164 | ICAM1 |
| | | | | SEQ ID NO: 67 | | | |
| 137** | HRV-B | 91 | ATCC | hrv-91 | hrv-91 | FJ445168 | ICAM1 |
| | | | | SEQ ID NO: 72 | | | |
| 138** | HRV-B | 92 | ATCC | hrv-92 | hrv-92 | FJ445169 | ICAM1 |
| | | | | SEQ ID NO: 73 | | | |
| 139 | HRV-B | 93 | SF-1492 | ef173425-93 | hrv-93* | EF173425 | ICAM1 |
| 140** | HRV-B | 97 | ATCC | hrv-97 | hrv-97 | FJ445172 | ICAM1 |
| | | | | SEQ ID NO: 77 | | | |
| 141** | HRV-B | 99 | ATCC | hrv-99 | hrv-99 | FJ445174 | ICAM1 |
| | | | | SEQ ID NO: 79 | | | |

| msf | | | | | | | | published |
|---|---|---|---|---|---|---|---|---|
| file | drug | ORF | ORF | protein | seq | | seq | sequence |
| order (1) | group (9) | start (10) | end (11) | length (12) | length (13) | 3′NTR (14) | desc (15) | reference |
| — | — | — | — | — | — | — | — | (17-21) |
| 1 | 1 | 746 | 7387 | 2214 | 7458 | 71 | cg | (22) |
| 2 | 1 | 714 | 7334 | 2207 | 7406 | 72 | cg | (22) |
| 3 | 1 | 743 | 7369 | 2209 | 7440 | 71 | cg | (23) |
| 4 | 2 | 623 | 7093 | 2157 | 7133 | 40 | cg | (24) |
| 5 | 2 | 627 | 7097 | 2157 | 7137 | 40 | cg | Pico-DB(7) |
| 6** | 2 | 627 | 7097 | 2157 | 7137 | 40 | cg | This study |
| 7 | 2 | 611 | 7060 | 2150 | 7102 | 42 | cg | (25) |
| 8** | 2 | 619 | 7104 | 2162 | 7146 | 42 | cg | This study |
| 9 | 2 | 619 | 7104 | 2162 | 7146 | 42 | cg | (26) |
| 10** | 2 | 607 | 7068 | 2154 | 7108 | 40 | cg | This study |
| 11 | 2 | 611 | 7081 | 2157 | 7128 | 47 | cg | Pico-DB(7) |
| 12** | 2 | 612 | 7085 | 2158 | 7132 | 47 | cg | This study |
| 13** | 2 | 614 | 7087 | 2158 | 7134 | 47 | cg | This study |
| 14** | 2 | 613 | 7086 | 2158 | 7133 | 47 | cg | This study |
| 15 | 2 | 609 | 7088 | 2160 | 7137 | 49 | cg | (26) |
| 16** | 2 | 609 | 7088 | 2160 | 7137 | 49 | cg | This study |
| 17 | 2 | 619 | 7077 | 2153 | 7125 | 48 | cg | (27) |
| 18 | 2 | 614 | 7078 | 2155 | 7124 | 46 | cg | (27) |
| 19** | 1 | 616 | 7095 | 2160 | 7140 | 45 | cg | This study |
| 20** | 1 | 617 | 7096 | 2160 | 7143 | 47 | cg | This study |
| 21 | 2 | 616 | 7092 | 2159 | 7134 | 42 | cg | (26) |
| 22 | 2 | 626 | 7084 | 2153 | 7124 | 40 | cg | (28) |
| 23** | 2 | 612 | 7070 | 2153 | 7119 | 49 | cg | This study |
| 24** | 2 | 615 | 7088 | 2158 | 7135 | 47 | cg | This study |
| 25** | 2 | 622 | 7119 | 2166 | 7163 | 44 | cg | This study |
| 26** | 2 | 615 | 7085 | 2157 | 7134 | 49 | cg | This study |
| 27** | 2 | 615 | 7082 | 2156 | 7129 | 47 | cg | This study |
| 28 | 2 | 535 | 6981 | 2149 | 7025 | 44 | cg-5 | (26) |
| 29** | 2 | 622 | 7083 | 2154 | 7132 | 49 | cg | This study |
| 30 | 2 | 622 | 7083 | 2154 | 7132 | 49 | cg | (27) |
| 31** | 2 | 613 | 7077 | 2155 | 7126 | 49 | cg | This study |
| 32 | 2 | 622 | 7104 | 2161 | 7148 | 44 | cg | (26) |
| 33** | 2 | 613 | 7074 | 2154 | 7123 | 49 | cg | This study |
| 34 | 2 | 609 | 7055 | 2149 | 7099 | 44 | cg | (26) |
| 35** | 2 | 609 | 7055 | 2149 | 7099 | 44 | cg | This study |
| 36** | 2 | 612 | 7082 | 2157 | 7131 | 49 | cg | This study |
| 37** | 1 | 611 | 7084 | 2158 | 7133 | 49 | cg | This study |
| 38** | 2 | 620 | 7084 | 2155 | 7133 | 49 | cg | This study |
| 39** | 2 | 612 | 7070 | 2153 | 7119 | 49 | cg | This study |
| 40 | 2 | 612 | 7070 | 2153 | 7119 | 49 | cg | (26) |
| 41 | 2 | 617 | 7099 | 2161 | 7141 | 42 | cg | (26) |
| 42** | 2 | 611 | 7087 | 2159 | 7136 | 49 | cg | This study |
| 43 | 2 | 611 | 7087 | 2159 | 7136 | 49 | cg | (26) |
| 44 | 2 | 615 | 7085 | 2157 | 7136 | 51 | cg | (29) |
| 45** | 2 | 613 | 7089 | 2159 | 7138 | 49 | cg | This study |
| 46 | 2 | 616 | 7098 | 2161 | 7145 | 47 | cg | (26) |
| 47** | 1 | 614 | 7087 | 2158 | 7129 | 42 | cg | This study |
| 48 | 2 | 613 | 7074 | 2154 | 7123 | 49 | cg | (26) |
| 49** | 2 | 616 | 7071 | 2152 | 7114 | 43 | cg | This study |
| 50 | 2 | 613 | 7104 | 2164 | 7149 | 45 | cg | (26) |
| 51** | 2 | 613 | 7083 | 2157 | 7132 | 49 | cg | This study |
| 52 | 2 | 613 | 7062 | 2150 | 7106 | 44 | cg | (26) |
| 53** | 2 | 616 | 7065 | 2150 | 7109 | 44 | cg | This study |
| 54** | 2 | 611 | 7069 | 2153 | 7118 | 49 | cg | This study |
| 55** | 2 | 614 | 7108 | 2165 | 7152 | 44 | cg | This study |
| 56 | 2 | 613 | 7098 | 2162 | 7143 | 45 | cg | (26) |
| 57** | 1 | 612 | 7085 | 2158 | 7134 | 49 | cg | This study |
| 58** | 1 | 611 | 7084 | 2158 | 7133 | 49 | cg | This study |
| 59 | 2 | 523 | 6987 | 2155 | 7036 | 49 | cg-5 | (26) |
| 60** | 2 | 614 | 7087 | 2158 | 7136 | 49 | cg | This study |
| 61** | 2 | 615 | 7085 | 2157 | 7134 | 49 | cg | This study |
| 62** | 2 | 619 | 7098 | 2160 | 7140 | 42 | cg | This study |
| 63 | 2 | 612 | 7085 | 2158 | 7135 | 50 | cg | (26) |
| 64** | 2 | 614 | 7090 | 2159 | 7139 | 49 | cg | This study |
| 65** | 2 | 619 | 7098 | 2160 | 7139 | 41 | cg | This study |
| 66** | 2 | 615 | 7082 | 2156 | 7131 | 49 | cg | This study |
| 67** | 2 | 619 | 7092 | 2158 | 7141 | 49 | cg | This study |
| 68** | 2 | 615 | 7082 | 2156 | 7129 | 47 | cg | This study |
| 69 | 2 | 615 | 7082 | 2156 | 7129 | 47 | cg | (27) |
| 70** | 2 | 624 | 7118 | 2165 | 7162 | 44 | cg | This study |
| 71** | 2 | 617 | 7090 | 2158 | 7139 | 49 | cg | This study |
| 72** | 2 | 612 | 7085 | 2158 | 7135 | 50 | cg | This study |
| 73** | 2 | 623 | 7120 | 2166 | 7164 | 44 | cg | This study |
| 74** | 2 | 626 | 7117 | 2164 | 7161 | 44 | cg | This study |
| 75 | 2 | 616 | 7095 | 2160 | 7140 | 45 | cg | (26) |
| 76 | 2 | 611 | 7078 | 2156 | 7120 | 42 | cg | (26) |
| 77 | 2 | 618 | 7091 | 2158 | 7137 | 46 | cg | (26) |
| 78 | 2 | 620 | 7084 | 2155 | 7129 | 45 | cg | (26) |
| 79** | 2 | 619 | 7083 | 2155 | 7128 | 45 | cg | This study |
| 80** | 2 | 614 | 7087 | 2158 | 7136 | 49 | cg | This study |
| 81** | 2 | 623 | 7099 | 2159 | 7145 | 46 | cg | This study |
| 82 | 2 | 623 | 7099 | 2159 | 7145 | 46 | cg | (27) |
| 83** | 2 | 616 | 7095 | 2160 | 7138 | 43 | cg | This study |
| 84** | 1 | 618 | 7076 | 2153 | 7116 | 40 | cg | This study |
| 85** | 1 | 618 | 7076 | 2153 | 7116 | 40 | cg | This study |
| 86** | 1 | 618 | 7076 | 2153 | 7116 | 40 | cg | This study |
| 87** | 2 | 615 | 7079 | 2155 | 7123 | 44 | cg | This study |
| 88 | 2 | 621 | 7085 | 2155 | 7129 | 44 | cg | (26) |
| 89** | 2 | 615 | 7091 | 2159 | 7140 | 49 | cg | This study |
| 90 | 2 | 615 | 7091 | 2159 | 7140 | 49 | cg | Pico-DB(7) |
| 91 | 2 | 622 | 7101 | 2160 | 7143 | 42 | cg | (26) |
| 92 | 2 | 619 | 7110 | 2164 | 7152 | 42 | cg | US patent |
| | | | | | | | | DE3628658- |
| | | | | | | | | A1 Mar. 03, |
| | | | | | | | | 1988 |
| 93** | 2 | 619 | 7110 | 2164 | 7152 | 42 | cg | This study |
| 94** | 2 | 617 | 7108 | 2164 | 7150 | 42 | cg | This study |
| 95** | 2 | 619 | 7110 | 2164 | 7152 | 42 | cg | This study |
| 96 | 2 | 619 | 7110 | 2164 | 7152 | 42 | cg | (30) |
| 97** | 2 | 617 | 7075 | 2153 | 7124 | 49 | cg | This study |
| 98** | 2 | 616 | 7083 | 2156 | 7132 | 49 | cg | This study |
| 99 | 2 | 616 | 7083 | 2156 | 7132 | 49 | cg | (27) |
| 100** | 2 | 609 | 7070 | 2154 | 7110 | 40 | cg | This study |
| 101** | 2 | 618 | 7088 | 2157 | 7134 | 46 | cg | This study |
| 102** | 2 | 611 | 7084 | 2158 | 7133 | 49 | cg | This study |
| 103** | 2 | 612 | 7091 | 2160 | 7140 | 49 | cg | This study |
| 104 | na | 438 | 6866 | 2143 | 6917 | 51 | cg-5 | (31) |
| 105 | na | 614 | 7039 | 2142 | 7079 | 40 | cg | (26) and this |
| | | | | | | | | study |
| 106 | na | 609 | 7025 | 2139 | 7072 | 47 | cg | (32, 33) |
| 107 | na | 616 | 7047 | 2144 | 7099 | 52 | cg | (34) |
| 108 | na | 542 | 6973 | 2144 | 7015 | 42 | cg-5 | (26) |
| 109 | na | 612 | 7037 | 2142 | 7086 | 49 | cg | (34) |
| 110 | na | 617 | 7072 | 2152 | 7114 | 42 | cg | (34) |
| 111 | 1 | 627 | 7160 | 2178 | 7208 | 48 | cg | (26) |
| 112 | 1 | 627 | 7160 | 2178 | 7211 | 51 | cg | (27) |
| 113 | 1 | 626 | 7156 | 2177 | 7212 | 56 | cg | (26) |
| 114** | 1 | 632 | 7162 | 2177 | 7212 | 50 | cg | This study |
| 115 | 1 | 628 | 7164 | 2179 | 7216 | 52 | cg | (26) |
| 116 | 1 | 629 | 7165 | 2179 | 7212 | 47 | cg | (35) |
| 117 | 1 | 629 | 7165 | 2179 | 7212 | 47 | cg | (36) |
| 118 | 1 | 619 | 7176 | 2186 | 7219 | 43 | cg | (27) |
| 119** | 1 | 633 | 7160 | 2176 | 7211 | 51 | cg | This study |
| 120** | 1 | 628 | 7161 | 2178 | 7217 | 56 | cg | This study |
| 121 | 1 | 628 | 7161 | 2178 | 7217 | 56 | cg | (27) |
| 122 | 1 | 623 | 7168 | 2182 | 7224 | 56 | cg | (26) |
| 123** | 1 | 623 | 7168 | 2182 | 7224 | 56 | cg | This study |
| 124 | 1 | 631 | 7164 | 2178 | 7216 | 52 | cg | (27) |
| 125** | 1 | 633 | 7163 | 2177 | 7223 | 60 | cg | This study |
| 126 | 1 | 623 | 7174 | 2184 | 7214 | 40 | cg | (26) |
| 127** | 1 | 625 | 7173 | 2183 | 7216 | 43 | cg | This study |
| 128** | 1 | 625 | 7173 | 2183 | 7216 | 43 | cg | This study |
| 129 | 1 | 625 | 7173 | 2183 | 7216 | 43 | cg | (27) |
| 130** | 1 | 621 | 7169 | 2183 | 7211 | 42 | cg | This study |
| 131 | 1 | 623 | 7180 | 2186 | 7223 | 43 | cg | (26) |
| 132** | 1 | 631 | 7167 | 2179 | 7216 | 49 | cg | This study |
| 133** | 1 | 625 | 7170 | 2182 | 7224 | 54 | cg | This study |
| 134** | 1 | 627 | 7175 | 2183 | 7230 | 55 | cg | This study |
| 135** | 1 | 622 | 7152 | 2177 | 7201 | 49 | cg | This study |
| 136** | 1 | 632 | 7165 | 2178 | 7213 | 48 | cg | This study |
| 137** | 1 | 621 | 7178 | 2186 | 7221 | 43 | cg | This study |
| 138** | 1 | 628 | 7176 | 2183 | 7233 | 57 | cg | This study |
| 139 | 1 | 629 | 7162 | 2178 | 7215 | 53 | cg | (27) |
| 140** | 1 | 626 | 7156 | 2177 | 7207 | 51 | cg | This study |
| 141** | 1 | 626 | 7156 | 2177 | 7208 | 52 | cg | This study |

Table 1 Legend
(1) Order in which these sequences are listed in accompanying protein alignment (Table 5).
(2) Designation of Human Rhinovirus-C (HRV-C) as a species has been accepted by the ICTV Study Group on Picornaviruses.
(3) Field strains were assigned serotypes on the basis of sequence similarity to reference (ATCC) strains.
(4) Strain designation from GenBank sequence description if available; ATCC origin; or field-strain isolate.
(5) Protein and RNA alignments use these names. Accession number, if available, precedes serotype; f01-f10: field strains; entries beginning with “hrv-” are from this study.
(6) (abbreviated) Strain names used on phylograms. Not all alignment entries are included (not inc). Asterisk (*) designates GenBank origin, sequence is not original to this study.
(7) GenBank accession number for published sequences (black) or from this study (green). “Pico-DB” is unpublished, but available at: http://www.picornaviridae.com/
(8) The Major (M) group HRV use ICAM-1 receptors. The Minor (m) group HRVs use LDL receptors. See reference: (35).
(9) Drug group reactivity according to reference: (36). na: not part of that study.
(10) 1^{st} base of polyprotein open reading frame within individual sequence file.
(11) Last base of polyprotein open reading frame within individual sequence file. Count excludes the ORF termination codon.
(12) Polyprotein length, in amino acids, encoded by this genome.
(13) Genome sequence length.
(14) 3′ non-coding fragment length. Count does not include poly(A).
(15) cg: complete genome. cg-5: almost complete genome, except for 5′ end.
(16) Chimeric sequence: bases 1-135 from this study, remainder from EF077279.
(17) Thomas Briese and Ian Lipkin, personal communication to ACP.
(18) First entry of Table 5 alignment includes an annotation line with protein structure features.
Citations from Table 1
17. T. C. Bjorndahl, H. Monzavi, D. S. Wishart, J. Biomol. NMR 26, 85 (2003).
18. E. Arnold, M. G. Rossmann, J. Mol. Biol. 211, 763 (1990).
19. D. W. Gohara et al., J. Biol. Chem. 275, 25523 (2000).
20. J. F. Petersen et al., EMBO J. 18, 5463 (1999).
21. D. M. Strauss, L. W. Glustrom, D. S. Wuttke, J. Mol. Biol. 330, 225 (2003).
22. B. Brown, M. S. Oberste, K. Maher, M. A. Pallansch, J. Virol. 77, 8973 (2003).
23. V. R. Racaniello, D. Baltimore, Proc. Natl. Acad. Sci. U.S.A. 78, 4887 (1981).
24. P. J. Hughes, C. North, C. H. Jellis, P. D. Minor, G. Stanway, J. Gen. Virol. 69 (Pt 1), 49 (1988).
25. T. Skern et al., Nucleic Acids Res. 13, 2111 (1985).
26. A. L. Kistler et al., Virol. J. 4, 40 (2007).
27. C. Tapparel et al., BMC Genomics 8, 224 (2007).
28. W. M. Lee, W. Wang, R. R. Rueckert, Virus Genes 9, 177 (1995).
29. J. R. Harris, V. R. Racaniello, J. Virol. 79, 5363 (2005).
30. M. Duechler et al., Proc. Natl. Acad. Sci. U.S.A. 84, 2605 (1987).
31. P. McErlean et al., J. Clin. Virol. 39, 67 (2007).
32. N. Renwick et al., J. Infect. Dis. 196, 1754 (2007).
33. D. Lamson et al., J. Infect. Dis. 194, 1398 (2006).
34. S. K. Lau et al., J. Clin. Microbiol. 45, 3655 (2007).
35. T. J. Smith et al., J. Virol. 67, 1148 (1993).
36. G. Stanway, P. J. Hughes, R. C. Mountford, P. D. Minor, J. W. Almond, Nucleic Acids Res. 12, 7859 (1984).

TABLE 2
Recombination events in HRV serotypes. The P-value listed is
the lowest obtained. See Table 1 for full data and P-values.

| Major | Minor | Recom- | | |
|---|---|---|---|---|
| Parent | Parent | binant | Genome Region | P-value |
| hrv-45 | hrv-21 | hrv-8 | 5′-UTR | 1.094 × 10−21 |
| hrv-45 | hrv-21 | hrv-95 | 5′UTR | 1.094 × 10−21 |
| hrv-65 | hrv-21 | hrv-80 | 5′-UTR, VP4 | 6.129 × 10−23 |
| hrv-51 | hrv-11 | hrv-20 | 5′-UTR, VP4 | 8.402 × 10−26 |
| hrv-51 | hrv-11 | hrv-68 | 5′-UTR, VP4 | 8.402 × 10−26 |
| hrv-28 | hrv-62 | hrv-71 | 5′-UTR | 8.484 × 10−15 |
| hrv-54-f05 | hrv-75 | hrv-18 | 5′-UTR, VP4, VP2, | 3.737 × 10−7 |
| | | | VP3, VP1 | |
| hrv-54-f05 | hrv-75 | hrv-24 | 5-UTR, VP4, VP2, | 3.737 × 10−7 |
| | | | VP3, VP1 | |
| hrv-54-f05 | hrv-75 | hrv-50 | 5′-UTR, VP4, VP2, | 3.737 × 10−7 |
| | | | VP3, VP1 , P2A | |
| hrv-54-f05 | hrv-75 | hrv-34 | VP4, VP2, VP3, | 3.737 × 10−7 |
| | | | VP1 | |
| hrv-54-f05 | hrv-67 | hrv-38 | VP2, VP3, VP1, | 1.561 × 10−29 |
| | | | P2A, P2B, P2C, | |
| | | | P3A | |
| hrv-54 | hrv-67 | hrv-60 | VP2, VP3, VP1, | 1.561 × 10−29 |
| | | | P2A, P2B, P2C, | |
| | | | P3A | |
| hrv-53 | hrv-80 | hrv-46 | 5′-UTR, VP4, VP2, | 1.291 × 10−62 |
| | | | VP3, VP1 | |
| hrv-13-f03 | HRV41 | hrv-73 | 5′-UTR, VP4, VP2, | 9.419 × 10−12 |
| | | | VP3, VP1 | |
| hrv-30 | hrv-59 | hrv-39 | 5′-UTR, VP4, VP2, | 2.963 × 10−12 |
| | | | VP3, VP1 | |
| hrv-68 | hrv-20 | hrv-28 | VP4, VP2, VP3, | 6.839 × 10−12 |
| | | | VP1, P2A, P2B, | |
| | | | P2C | |
| hrv-100 | hrv-10 | hrv-56 | P2A, P2B, P2C, | 4.230 × 10−17 |
| | | | P3A, P3B, P3C, | |
| | | | P3D | |
| hrv-29 | hrv-54-f05 | hrv-31 | P3C, P3D | 7.934 × 10−13 |
| hrv-29 | hrv-54-f05 | hrv-47 | P3C, P3D | 1.232 × 10−10 |
| hrv-42 | hrv-97 | hrv-4 | 5′-UTR, VP4 | 8.233 × 10−35 |

TABLE 3
Recombination analysis based on the HHM-based alignment using the complete
genomes from the reference set and the field samples. Results are shown for all
recombination events with P < 0.00001 by at least 2 analysis modes (see
Methods). P-values represent the results using the indicated analysis modes. The
nucleotide is referenced to the recombinant (daughter) hrv. See FIG. 3 for
representative recombinations in graphical forms. When a progressive alignment
was performed and recombination tested, 19 of the 23 recombinants were confirmed
(a, major and minor parents were the same as with HHM; b, one or both parents differed
from the HHM-based analysis).

| Major | Minor | | | | | |
|---|---|---|---|---|---|---|
| Parent | Parent | Recombinant | start | end | Genome Region | P-RDP |
| hrv-45 | hrv-21 | hrv-8 (a) | 13 | 631 | 5′-UTR | 3.351 × 10−20 |
| hrv-45 | hrv-21 | hrv-95 (a) | 34 | 599 | 5′UTR | 3.351 × 10−20 |
| hrv-65 | hrv-21 | hrv-80 (b) | 10 | 756 | 5′-UTR, VP4 | 1.077 × 10−21 |
| hrv-51 | hrv-11 | hrv-20 (b) | 6 | 690 | 5′-UTR, VP4 | 1.023 × 10−19 |
| hrv-51 | hrv-11 | hrv-68 (b) | 11 | 691 | 5′-UTR, VP4 | 1.023 × 10−19 |
| hrv-28 | hrv-62 | hrv-71 | 1 | 661 | 5′-UTR | 2.582 × 10−9 |
| hrv-54-f05 | hrv-75 | hrv-18 | 511 | 3058 | 5′-UTR, VP4, VP2, VP3, VP1 | 1.371 × 10−3 |
| hrv-54-f05 | hrv-75 | hrv-24 (b) | 540 | 3071 | 5-UTR, VP4, VP2, VP3, VP1 | 1.371 × 10−3 |
| hrv-54-f05 | hrv-75 | hrv-50 (b) | 554 | 3294 | 5′-UTR, VP4, VP2, VP3, VP1, | 1.371 × 10−3 |
| | | | | | P2A | |
| hrv-54-f05 | hrv-75 | hrv-34 | 651 | 3088 | VP4, VP2, VP3, VP1 | 1.371 × 10−3 |
| hrv-54-f05 | hrv-67 | hrv-38 (b) | 1006 | 4935 | VP2, VP3, VP1, P2A, P2B, | |
| | | | | | P2C, P3A | |
| hrv-54 | hrv-67 | hrv-60 (b) | 995 | 4979 | VP2, VP3, VP1, P2A, P2B, | 8.952 × 10−12 |
| | | | | | P2C, P3A | |
| hrv-53 | hrv-80 | hrv-46 (a) | 32 | 3222 | 5′-UTR, VP4, VP2, VP3, VP1 | 1.153 × 10−22 |
| hrv-13-f03 | HRV41 | hrv-73 | 64 | 3333 | 5′-UTR, VP4, VP2, VP3, VP1 | 1.606 × 10−2 |
| hrv-30 | hrv-59 | hrv-39 (b) | 450 | 3148 | 5′-UTR, VP4, VP2, VP3, VP1 | 1.958 × 10−7 |
| hrv-68 | hrv-20 | hrv-28 (a) | 691 | 4143 | VP4, VP2, VP3, VP1, P2A, | |
| | | | | | P2B, P2C | |
| hrv-100 | hrv-10 | hrv-56 (b) | 3487 | 6889 | P2A, P2B, P2C, P3A, P3B, | 4.315 × 10−3 |
| | | | | | P3C, P3D | |
| hrv-29 | hrv-54-f05 | hrv-31 (a) | 5181 | 7007 | P3C, P3D | 3.3 × 10−10 |
| hrv-29 | hrv-54-f05 | hrv-47 (a) | 5321 | 7008 | P3C, P3D | 3.3 × 10−10 |
| hrv-42 | hrv-97 | hrv-4 (a) | 1 | 640 | 5′-UTR, VP4 | 1.322 × 10−12 |
| hrv-84 | hrv-37 | hrv-27 (b) | 48 | 718 | 5′-UTR, VP4 | 7.842 × 10−4 |
| hrv-84 | hrv-37 | hrv-93 (b) | 100 | 772 | 5′-UTR, VP4 | 7.842 × 10−4 |
| hrv-84 | hrv-37 | hrv-97 (b) | 101 | 748 | 5′-UTR, VP4 | 7.842 × 10−4 |

| Major | | | | | |
|---|---|---|---|---|---|
| Parent | P-BootScan | P-MaxChi | P-Chimeara | P-Siscan | P-3Seq |
| hrv-45 | 1.094 × 10−21 | 5.518 × 10−08 | 4.7 × 10−9 | 6.061 × 10−12 | |
| hrv-45 | 1.094 × 10−21 | 5.518 × 10−08 | 4.7 × 10−9 | 6.061 × 10−12 | |
| hrv-65 | 6.129 × 10−23 | 9.417 × 10−8 | 1.584 × 10−3 | 5.099 × 10−12 | 2.235 × 10−5 |
| hrv-51 | 8.402 × 10−26 | 1.606 × 10−6 | 7.286 × 10−7 | 2.187 × 10−20 | 2.101 × 10−12 |
| hrv-51 | 8.402 × 10−26 | 1.606 × 10−6 | 7.286 × 10−7 | 2.187 × 10−20 | 2.101 × 10−12 |
| hrv-28 | 8.484 × 10−15 | 1.194 × 10−3 | 4.255 × 10−6 | | 1.899 × 10−8 |
| hrv-54-f05 | | 1.555 × 10−6 | 3.737 × 10−7 | 8.391 × 10−3 | 2.173 × 10−3 |
| hrv-54-f05 | | 1.555 × 10−6 | 3.737 × 10−7 | 8.391 × 10−3 | 2.173 × 10−3 |
| hrv-54-f05 | | 1.555 × 10−6 | 3.737 × 10−7 | 8.391 × 10−3 | 2.173 × 10−3 |
| hrv-54-f05 | | 1.555 × 10−6 | 3.737 × 10−7 | 8.391 × 10−3 | 2.173 × 10−3 |
| hrv-54-f05 | 1.561 × 10−29 | 9.616 × 10−14 | 1.322 × 10−14 | 8.465 × 10−5 | 3.552 × 1−18 |
| hrv-54 | 1.561 × 10−29 | 9.616 × 10−14 | 1.322 × 10−14 | 8.465 × 10−5 | 3.552 × 1-0−18 |
| hrv-53 | 2.641 × 10−16 | 3.295 × 10−23 | 1.261 × 10−25 | 3.511 × 10−10 | 1.291 × 10−62 |
| hrv-13-f03 | | 3.503 × 10−9 | 9.419 × 10−12 | | 2.363 × 10−8 |
| hrv-30 | 6.012 × 10−3 | 9.356 × 10−11 | 2.963 × 10−12 | 8.613 × 10−7 | 1.023 × 10−11 |
| hrv-68 | 4.245 × 10−11 | 9.232 × 10−6 | 1.932 × 10−6 | 6.839 × 10−12 | |
| hrv-100 | 4.230 × 10−17 | 1.23 × 10−7 | 1.921 × 10−10 | | 5.416 × 10−7 |
| hrv-29 | 7.934 × 10−13 | 1.038 × 10−6 | 5.174 × 10−8 | 1.232 × 10−10 | |
| hrv-29 | | 1.038 × 10−6 | 5.174 × 10−8 | 1.232 × 10−10 | |
| hrv-42 | 8.233 × 10−35 | 1.315 × 10−10 | 1.126 × 10−8 | 9.599 × 10−22 | 4.975 × 10−4 |
| hrv-84 | 1.500 × 10−10 | 9.948 × 10−4 | 1.061 × 10−3 | 2.364 × 10−10 | 1.792 × 10−4 |
| hrv-84 | 1.500 × 10−10 | 9.948 × 10−4 | 1.061 × 10−3 | 2.364 × 10−10 | 1.792 × 10−4 |
| hrv-84 | 1.500 × 10−10 | 9.948 × 10−4 | 1.061 × 10−3 | 2.364 × 10−10 | 1.792 × 10−4 |

TABLE 4
Amino acid differences between the HRV reference genomes and the analogous field isolates.
The position is the amino acid number of the reference genome for each comparison.

| | hrv-52-f10 | hrv-89-f08 | hrv-89-f09 | hrv-81-f06 | hrv-81-f07 | hrv-13-f03 | hrv-09-f01 | hrv-09-f02 | hrv-49-f04 | hrv-54-f05 |
|---|---|---|---|---|---|---|---|---|---|---|
| 40 | | | | | | | R40G | R40G | | |
| 59 | | | | | | | S59D | S59D | | |
| 136 | | | | | | K136E | | | | E136D |
| 142 | | | | N142S | N142S | | G142N | G142N | | |
| 143 | | | | | | | D143E | D143E | | |
| 156 | | | | | | | | | R156K | |
| 173 | | | | | | C173S | | | | |
| 193 | | | | | | | | | | V193I |
| 199 | | | | | | | D199H | D199H | | |
| 215 | | | | | | | | | N215K | |
| 216 | | | | | | | | | −f216Y | |
| 222 | Q222S | | | | | | | | T222A | |
| 227 | N227D | | | | | | | | | |
| 229 | I229T | | | | | I229T | | | | −229P |
| 230 | | | | | | N230G | | | | A230- |
| 232 | E232A | | T232S | | | | | | S232N | V232T |
| 233 | | | | | | | | | M233I | |
| 234 | | | | | | | | | | T234I |
| 237 | | | | K237R | K237R | | | | L237V | N237H |
| 271 | | | | | | | | | | T271S |
| 305 | | | | | | | | | | P305S |
| 308 | N308S | | | | | | | | | |
| 310 | T310S | | | | | | | | | |
| 335 | | S335N | S335N | | | | | | | |
| 339 | V339I | | | | | | | | | |
| 340 | P340- | | | | | | | | | |
| 344 | −344K | | | | | | | | S344N | |
| 351 | | | | | | | | | | V351I |
| 404 | | | | | | | | | | −f404L |
| 405 | T405V | | | T405A | T405A | | | | | A405P |
| 406 | | | | | | N406T | | | | |
| 410 | S410T | | | | | | | | | |
| 411 | | | | | | | | | E411G | |
| 412 | | | | | | | | | N412D | N412S |
| 417 | | | | | | | | | V417I | |
| 419 | | | | | | | | | | −f419S |
| 421 | | | | | | | Q421T | Q421T | | |
| 422 | K422R | | | | | | | | V422I | |
| 428 | | | | | | | T428S | T428S | | |
| 474 | | | | | | | S474T | S474T | | |
| 487 | S487A | | | | | | | | | |
| 488 | | | | | | | | | A488R | |
| 491 | | K491R | K491R | | | | T491I | | | |
| 494 | | | | | | | | | K494R | |
| 506 | | | | | | | | | V506I | |
| 526 | −f526Y | | | | | | | | | |
| 529 | | | | | | | | | P529S | |
| 531 | | | | | | | | | T531A | |
| 534 | | | | X534S | X534S | | | | | |
| 535 | | | | | | | | | S535A | |
| 538 | | | | | | | | | I538V | |
| 554 | | | | | | | | | A554P | |
| 555 | | | | | | | | | | S555N |
| 584 | | | | | | | | | V584P | |
| 586 | | | | T586E | T586E | | | | | |
| 589 | −f589L | | | | | | | | | |
| 592 | | | | | | | | | H592Q | R592K |
| 625 | | | | | | S625K | | | | |
| 626 | P626S | | | | | S626P | | | | |
| 631 | | | | S631A | S631A | | | | | |
| 656 | | | | | | | | | | −f656Y |
| 662 | T662N | | | | | | | | | |
| 675 | | | | S675A | S675A | | | | | |
| 679 | | | | | | H679Y | | | | |
| 681 | | | | | | S681H | | | | |
| 685 | | | | | | | I685V | I685V | | |
| 686 | E686K | | | | | | | | | Q686R |
| 687 | I687V | | | | | | | | | |
| 688 | | | | | | T688D | S688D | S688D | | |
| 689 | | | | | | N689A | | | | |
| 690 | N690D | T690V | T690V | | | | | | | |
| 696 | T696G | | | | | | | | | |
| 706 | | | | | | | E706K | E706K | | |
| 709 | S709N | | | | | | | | | |
| 713 | V713I | V713I | V713I | | | | | | | |
| 714 | N714S | | | | | | | | | |
| 721 | | | | | | | L721I | L721I | | |
| 725 | | Y725-f | Y725-f | | | | | | | |
| 734 | | | | N734D | N734D | | | | | |
| 745 | | A745T | A745T | | | | | | | |
| 749 | T749A | Q749K | Q749K | | | | | | | |
| 751 | Q751E | N751D | N751D | | | | | | K751E | V751A |
| 753 | | S753N | S753N | | | | | | | |
| 772 | E772K | | | | | | | | | |
| 786 | | | | | | | | | V786I | |
| 802 | V802I | | | | | | | | | |
| 827 | | | S827N | | | | | | Q827E | |
| 828 | A828P | | | | | S828T | | | | |
| 829 | N829E | | | H829Q | H829Q | | | | | |
| 836 | | | | | | I836V | | | | |
| 838 | | | | X838N | X838N | | | | | |
| 847 | | I847M | I847M | | | | | | | |
| 853 | L853-f | | | | | | | | | K853E |
| 855 | | | | | | | | | S855N | |
| 856 | | S856L | S856L | | | | | | | |
| 858 | | | | I858V | I858V | | | | | |
| 869 | | | | | | V869I | | | | |
| 892 | | | | | | | | | V892I | |
| 893 | N893S | S893L | S893L | | | G893K | | | E893Q | R893Q |
| 894 | | | | | | | T894E | T894E | D894N | E894D |
| 896 | | | | | | | D896E | D896E | | D896E |
| 898 | | | | | | | A898S | A898S | | |
| 911 | | | | K911R | K911R | A911T | | | T911K | |
| 914 | D914E | | | | | | | | A914P | T914E |
| 915 | | | | | | | | | | N915T |
| 916 | | V916I | V916I | | | | | | | |
| 917 | | | | | | | K917R | K917R | | V917T |
| 922 | N922S | | | | | | | | | |
| 923 | S923P | | | | | | | | | |
| 928 | V928I | | | | | | | | | |
| 948 | | | | | | | N948D | N948D | | |
| 952 | | | | | | | | | | V952I |
| 959 | | I959V | I959V | | | | | | | |
| 966 | | | | | | | I966T | I966T | | |
| 973 | | | | | | S973A | | | | |
| 987 | | | | | | | | | N987S | |
| 993 | | R993K | R993K | | | E993N | | | | |
| 995 | E995D | | | | | | | | | |
| 1035 | M1035V | | | | | | | | | |
| 1041 | | | | | | | I1041M | | | |
| 1042 | | I1042V | I1042V | | | | | | | |
| 1071 | | | | | | | | | I1071V | |
| 1092 | | | | D1092E | D1092E | | D1092E | D1092E | | |
| 1102 | | | | | | | S1102N | S1102N | | S1102N |
| 1105 | | | | | | | | | V1105I | |
| 1109 | | | | | | | | | | V1109I |
| 1110 | | | | | | | V1110I | V1110I | | |
| 1164 | | | | I1164V | I1164V | | | | | |
| 1209 | R1209K | | | | | | | | | |
| 1223 | | | | | | | | | | −f1223L |
| 1229 | A1229T | | | | | | | | | |
| 1233 | | | | | | | | | V1233I | |
| 1236 | | | | A1236T | A1236T | | | | | |
| 1238 | | V1238D | V1238D | S1238T | S1238T | | | | | |
| 1246 | | −f1246Y | −f1246Y | | | | | | | |
| 1249 | | | | | | I1249V | | | | |
| 1265 | | | | | | | | | G1265S | |
| 1272 | | | | | | | | | | V1272I |
| 1274 | | | | | | | | | | Y1274H |
| 1277 | V1277M | | | | | | | | | |
| 1278 | | | | | | N1278S | | | | |
| 1294 | | | | | | | | | I1294V | |
| 1299 | | | | | | L1299S | | | | |
| 1305 | | | | | | | P1305S | P1305S | | |
| 1412 | | | | | | | Y1412-f | Y1412-f | | |
| 1418 | | | | | | | V1418I | V1418I | I1418L | |
| 1431 | | | | | | | N1431D | N1431D | | |
| 1432 | A1432V | | | | | | | | | |
| 1437 | | | | | | R1437K | | | | |
| 1440 | | | | | | | | | N1440D | |
| 1441 | | | | | | V1441I | | | | |
| 1442 | | | | | | K1442D | | | | |
| 1443 | | I1443T | I1443T | | | | | | | |
| 1450 | K1450R | | | | | | | | | |
| 1460 | V1460I | | | | | | | | −f1460V | |
| 1461 | | | | | | | | | | V1461M |
| 1467 | | | | | | | | | S1467T | |
| 1469 | | | | R1469K | R1469K | | | | | |
| 1474 | X1474D | | | | | | | | | |
| 1478 | | | | N1478S | N1478S | | | | | |
| 1482 | | | | | | E1482Q | | | | |
| 1491 | | | | V1491I | V1491I | | | | | |
| 1514 | | | | | | V1514A | L1514S | L1514S | | |
| 1521 | | | | | | T1521A | | | | |
| 1530 | K1530E | | | | | | | | | |
| 1538 | | | | | | | | | V1538D | Y1538D |
| 1542 | | | | | | | T1542I | T1542I | | |
| 1550 | | | | | | K1550R | | | | |
| 1556 | | | | N1556S | N1556S | | | | | |
| 1560 | | | | N1560S | N1560S | | | | | |
| 1563 | | | | | | A1563T | | | | |
| 1569 | | | | I1569V | | | | | | |
| 1572 | | | | | | | V1572A | V1572A | | |
| 1600 | | | | I1600V | I1600V | | | | | |
| 1630 | | | | | | | | | E1630D | |
| 1645 | | | | | | I1645V | | | | |
| 1646 | | | | | | | V1646I | V1646I | | |
| 1658 | | V1658I | V1658I | | | | | | | |
| 1662 | | | | H1662Q | H1662Q | A1662T | | | | |
| 1669 | | | | | | | | | Y1669-f | |
| 1672 | | | | Y1672-f | Y1672-f | | | | | |
| 1674 | | | | | | | | | | R1674K |
| 1678 | | | | | | | N1678K | N1678K | | |
| 1682 | | | | | | S1682T | | | | |
| 1687 | | I1687A | I1687A | | | | | | | |
| 1718 | | | | | | | I1718D | I1718D | | |
| 1729 | | | V1729E | | I1729V | | | | I1729V | |
| 1748 | | | | | | | | | S1748N | |
| 1766 | | | | I1766V | I1766V | | | | | |
| 1776 | | | | | | | | | | K1776R |
| 1785 | K1785R | | | | | | | | | |
| 1796 | | | | | | | I1796V | I1796V | I1796V | |
| 1797 | | | | | | | N1797S | N1797S | | |
| 1798 | | | | | | T1798A | | | | |
| 1799 | | | | Q1799K | Q1799K | | | | | |
| 1800 | | | | | | | | | | P1800S |
| 1802 | | | | | | T1802A | | | | |
| 1804 | V1804M | | | | | | L1804I | L1804I | | |
| 1809 | | V1809I | V1809I | | | | | | | |
| 1811 | | | | | | | | | N1811S | |
| 1823 | H1823Y | | | | | | | | | |
| 1825 | I1825V | | | V1825I | V1825I | | | | | |
| 1836 | | | | | | | | | | |
| 1838 | T1838A | | | | | R1838K | K1838R | K1838R | | |
| 1840 | | | | | | | | | T1840A | |
| 1847 | C1847R | | | | | | | | | |
| 1855 | K1855R | | | | | | | | | |
| 1859 | | | | K1859E | K1859E | | | | | |
| 1860 | I1860T | | | | | | | | | |
| 1861 | | | | D1861E | D1861E | | | | −f1861S | |
| 1862 | | | | | | | | | | M1862I |
| 1866 | | | | M1866I | M1866I | | | | | |
| 1870 | | | | | | V1870I | | | | |
| 1883 | P1883S | | | | | | | | | |
| 1885 | E1885D | | | | | | D1885N | D1885N | | |
| 1887 | | | | I1887V | I1887V | | | | | |
| 1888 | | | | A1888T | A1888T | | | | | |
| 1906 | X1906T | | | | | | | | | |
| 1914 | | | | A1914S | A1914S | | | | | |
| 1920 | | | | K1920R | K1920R | | | | | |
| 1926 | | | K1926R | | | S1926T | N1926S | N1926S | | |
| 1928 | | K1928R | K1928R | | | | | | | |
| 1934 | | R1934K | R1934K | | | | | | | |
| 1935 | | | | | | | | | | M1935I |
| 1957 | R1957K | | | | | | | | | |
| 1961 | | | | P1961S | P1961S | | | | | S1961H |
| 1962 | | | | V1962A | V1962A | | | | | A1962T |
| 1973 | | | | | | | I1973V | I1973V | | |
| 1977 | | | | V1977I | V1977I | | | | | |
| 1978 | | | | | | | T1978A | T1978A | | |
| 1989 | | | | | | T1989S | | | | |
| 1992 | S1992L | | | | | | | | | |
| 1997 | | | | | | | | | I1997V | |
| 2008 | | | | | | | | | T2008I | |
| 2040 | R2040K | | | | | | | | | |
| 2047 | | | | | | I2047R | | | | |
| 2052 | R2052K | S2052P | | | | | | | | |
| 2055 | | | | | | | | | | S2055P |

TABLE 5
hrv08.pro.msf MSF: 2255 Type: P October 22, 2008 19:15 Check: 5838

| | | | |
|---|---|---|---|
| Name: annotation | Len: 2255 | Check: 4935 | Weight: 1.00 |
| Name: af499637-cva13 | Len: 2255 | Check: 447 | Weight: 1.00 |
| Name: af546702-cva21 | Len: 2255 | Check: 4642 | Weight: 1.00 |
| Name: v01149-pv1m | Len: 2255 | Check: 6327 | Weight: 1.00 |
| Name: d00239-01 | Len: 2255 | Check: 7463 | Weight: 1.00 |
| Name: pico-01 | Len: 2255 | Check: 6186 | Weight: 1.00 |
| Name: hrv-01 | Len: 2255 | Check: 6203 | Weight: 1.00 |
| Name: x02316-02 | Len: 2255 | Check: 7841 | Weight: 1.00 |
| Name: hrv-07 | Len: 2255 | Check: 5013 | Weight: 1.00 |
| Name: dq473503-07 | Len: 2255 | Check: 3087 | Weight: 1.00 |
| Name: hrv-08 | Len: 2255 | Check: 1247 | Weight: 1.00 |
| Name: pico-09 | Len: 2255 | Check: 6334 | Weight: 1.00 |
| Name: hrv-09 | Len: 2255 | Check: 7236 | Weight: 1.00 |
| Name: hrv-09-f01 | Len: 2255 | Check: 4151 | Weight: 1.00 |
| Name: hrv-09-f02 | Len: 2255 | Check: 4476 | Weight: 1.00 |
| Name: dq473498-10 | Len: 2255 | Check: 850 | Weight: 1.00 |
| Name: hrv-10 | Len: 2255 | Check: 850 | Weight: 1.00 |
| Name: ef173414-11 | Len: 2255 | Check: 8297 | Weight: 1.00 |
| Name: ef173415-12 | Len: 2255 | Check: 6532 | Weight: 1.00 |
| Name: hrv-13 | Len: 2255 | Check: 4848 | Weight: 1.00 |
| Name: hrv-13-f03 | Len: 2255 | Check: 6361 | Weight: 1.00 |
| Name: dq473493-15 | Len: 2255 | Check: 5079 | Weight: 1.00 |
| Name: l24917-16 | Len: 2255 | Check: 9066 | Weight: 1.00 |
| Name: hrv-18 | Len: 2255 | Check: 2115 | Weight: 1.00 |
| Name: hrv-19 | Len: 2255 | Check: 3752 | Weight: 1.00 |
| Name: hrv-20 | Len: 2255 | Check: 5241 | Weight: 1.00 |
| Name: hrv-21 | Len: 2255 | Check: 2811 | Weight: 1.00 |
| Name: hrv-22 | Len: 2255 | Check: 3661 | Weight: 1.00 |
| Name: dq473497-23 | Len: 2255 | Check: 2349 | Weight: 1.00 |
| Name: hrv-24 | Len: 2255 | Check: 8306 | Weight: 1.00 |
| Name: ef173416-24 | Len: 2255 | Check: 7278 | Weight: 1.00 |
| Name: hrv-25 | Len: 2255 | Check: 7535 | Weight: 1.00 |
| Name: dq473508-28 | Len: 2255 | Check: 1578 | Weight: 1.00 |
| Name: hrv-29 | Len: 2255 | Check: 5183 | Weight: 1.00 |
| Name: dq473512-30 | Len: 2255 | Check: 3262 | Weight: 1.00 |
| Name: hrv-30 | Len: 2255 | Check: 3559 | Weight: 1.00 |
| Name: hrv-31 | Len: 2255 | Check: 6949 | Weight: 1.00 |
| Name: hrv-32 | Len: 2255 | Check: 4105 | Weight: 1.00 |
| Name: hrv-33 | Len: 2255 | Check: 7247 | Weight: 1.00 |
| Name: hrv-34 | Len: 2255 | Check: 5118 | Weight: 1.00 |
| Name: dq473501-34 | Len: 2255 | Check: 4982 | Weight: 1.00 |
| Name: dq473505-36 | Len: 2255 | Check: 2273 | Weight: 1.00 |
| Name: hrv-38 | Len: 2255 | Check: 9028 | Weight: 1.00 |
| Name: dq473495-38 | Len: 2255 | Check: 8056 | Weight: 1.00 |
| Name: ay751783-39 | Len: 2255 | Check: 3443 | Weight: 1.00 |
| Name: hrv-40 | Len: 2255 | Check: 6843 | Weight: 1.00 |
| Name: dq473491-41 | Len: 2255 | Check: 7539 | Weight: 1.00 |
| Name: hrv-43 | Len: 2255 | Check: 7974 | Weight: 1.00 |
| Name: dq473499-44 | Len: 2255 | Check: 3531 | Weight: 1.00 |
| Name: hrv-45 | Len: 2255 | Check: 2115 | Weight: 1.00 |
| Name: dq473506-46 | Len: 2255 | Check: 6630 | Weight: 1.00 |
| Name: hrv-47 | Len: 2255 | Check: 6617 | Weight: 1.00 |
| Name: dq473496-49 | Len: 2255 | Check: 8506 | Weight: 1.00 |
| Name: hrv-49-f04 | Len: 2255 | Check: 7414 | Weight: 1.00 |
| Name: hrv-50 | Len: 2255 | Check: 6681 | Weight: 1.00 |
| Name: hrv-51 | Len: 2255 | Check: 7727 | Weight: 1.00 |
| Name: dq473507-53 | Len: 2255 | Check: 6412 | Weight: 1.00 |
| Name: hrv-54 | Len: 2255 | Check: 9861 | Weight: 1.00 |
| Name: hrv-54-f05 | Len: 2255 | Check: 6687 | Weight: 1.00 |
| Name: dq473511-55 | Len: 2255 | Check: 9894 | Weight: 1.00 |
| Name: hrv-56 | Len: 2255 | Check: 2611 | Weight: 1.00 |
| Name: hrv-57 | Len: 2255 | Check: 339 | Weight: 1.00 |
| Name: hrv-58 | Len: 2255 | Check: 9705 | Weight: 1.00 |
| Name: dq473500-59 | Len: 2255 | Check: 2882 | Weight: 1.00 |
| Name: hrv-60 | Len: 2255 | Check: 53 | Weight: 1.00 |
| Name: hrv-61 | Len: 2255 | Check: 9103 | Weight: 1.00 |
| Name: hrv-62 | Len: 2255 | Check: 7013 | Weight: 1.00 |
| Name: hrv-63 | Len: 2255 | Check: 4641 | Weight: 1.00 |
| Name: hrv-64 | Len: 2255 | Check: 5394 | Weight: 1.00 |
| Name: ef173417-64 | Len: 2255 | Check: 3160 | Weight: 1.00 |
| Name: hrv-65 | Len: 2255 | Check: 3638 | Weight: 1.00 |
| Name: hrv-66 | Len: 2255 | Check: 7130 | Weight: 1.00 |
| Name: hrv-67 | Len: 2255 | Check: 7013 | Weight: 1.00 |
| Name: hrv-68 | Len: 2255 | Check: 4503 | Weight: 1.00 |
| Name: hrv-71 | Len: 2255 | Check: 1844 | Weight: 1.00 |
| Name: dq473492-73 | Len: 2255 | Check: 5881 | Weight: 1.00 |
| Name: dq473494-74 | Len: 2255 | Check: 9321 | Weight: 1.00 |
| Name: dq473510-75 | Len: 2255 | Check: 2355 | Weight: 1.00 |
| Name: dq473502-76 | Len: 2255 | Check: 3906 | Weight: 1.00 |
| Name: hrv-76 | Len: 2255 | Check: 3651 | Weight: 1.00 |
| Name: hrv-77 | Len: 2255 | Check: 1505 | Weight: 1.00 |
| Name: hrv-78 | Len: 2255 | Check: 1027 | Weight: 1.00 |
| Name: ef173418-78 | Len: 2255 | Check: 369 | Weight: 1.00 |
| Name: hrv-80 | Len: 2255 | Check: 7222 | Weight: 1.00 |
| Name: hrv-81 | Len: 2255 | Check: 5990 | Weight: 1.00 |
| Name: hrv-81-f06 | Len: 2255 | Check: 5925 | Weight: 1.00 |
| Name: hrv-81-f07 | Len: 2255 | Check: 5782 | Weight: 1.00 |
| Name: hrv-82 | Len: 2255 | Check: 9248 | Weight: 1.00 |
| Name: dq473509-82 | Len: 2255 | Check: 9925 | Weight: 1.00 |
| Name: hrv-85 | Len: 2255 | Check: 854 | Weight: 1.00 |
| Name: pico-85 | Len: 2255 | Check: 1136 | Weight: 1.00 |
| Name: dq473504-88 | Len: 2255 | Check: 95 | Weight: 1.00 |
| Name: a10937-89 | Len: 2255 | Check: 3855 | Weight: 1.00 |
| Name: hrv-89 | Len: 2255 | Check: 4028 | Weight: 1.00 |
| Name: hrv-89-f08 | Len: 2255 | Check: 2571 | Weight: 1.00 |
| Name: hrv-89-f09 | Len: 2255 | Check: 2618 | Weight: 1.00 |
| Name: m16248-89 | Len: 2255 | Check: 3855 | Weight: 1.00 |
| Name: hrv-90 | Len: 2255 | Check: 4017 | Weight: 1.00 |
| Name: hrv-94 | Len: 2255 | Check: 5578 | Weight: 1.00 |
| Name: ef173419-94 | Len: 2255 | Check: 5531 | Weight: 1.00 |
| Name: hrv-95 | Len: 2255 | Check: 2386 | Weight: 1.00 |
| Name: hrv-96 | Len: 2255 | Check: 2237 | Weight: 1.00 |
| Name: hrv-98 | Len: 2255 | Check: 2787 | Weight: 1.00 |
| Name: hrv-100 | Len: 2255 | Check: 9900 | Weight: 1.00 |
| Name: ef186077-qpm | Len: 2255 | Check: 6593 | Weight: 1.00 |
| Name: ef077279-nat001 | Len: 2255 | Check: 536 | Weight: 1.00 |
| Name: ny1078 | Len: 2255 | Check: 1461 | Weight: 1.00 |
| Name: ef582385-c024 | Len: 2255 | Check: 3450 | Weight: 1.00 |
| Name: ef077280-nat045 | Len: 2255 | Check: 558 | Weight: 1.00 |
| Name: ef582387-c026 | Len: 2255 | Check: 7362 | Weight: 1.00 |
| Name: ef582386-c025 | Len: 2255 | Check: 255 | Weight: 1.00 |
| Name: dq473485-03 | Len: 2255 | Check: 5948 | Weight: 1.00 |
| Name: ef173422-03 | Len: 2255 | Check: 6362 | Weight: 1.00 |
| Name: dq473490-04 | Len: 2255 | Check: 8918 | Weight: 1.00 |
| Name: hrv-05 | Len: 2255 | Check: 3037 | Weight: 1.00 |
| Name: dq473486-06 | Len: 2255 | Check: 2318 | Weight: 1.00 |
| Name: l05355-14 | Len: 2255 | Check: 153 | Weight: 1.00 |
| Name: k02121-14 | Len: 2255 | Check: 86 | Weight: 1.00 |
| Name: ef173420-17 | Len: 2255 | Check: 3028 | Weight: 1.00 |
| Name: hrv-26 | Len: 2255 | Check: 7203 | Weight: 1.00 |
| Name: hrv-27 | Len: 2255 | Check: 815 | Weight: 1.00 |
| Name: ef173421-27 | Len: 2255 | Check: 815 | Weight: 1.00 |
| Name: dq473487-35 | Len: 2255 | Check: 3021 | Weight: 1.00 |
| Name: hrv-35 | Len: 2255 | Check: 2541 | Weight: 1.00 |
| Name: ef173423-37 | Len: 2255 | Check: 5738 | Weight: 1.00 |
| Name: hrv-42 | Len: 2255 | Check: 1573 | Weight: 1.00 |
| Name: dq473488-48 | Len: 2255 | Check: 2160 | Weight: 1.00 |
| Name: hrv-52 | Len: 2255 | Check: 4800 | Weight: 1.00 |
| Name: hrv-52-f10 | Len: 2255 | Check: 1413 | Weight: 1.00 |
| Name: ef173424-52 | Len: 2255 | Check: 5880 | Weight: 1.00 |
| Name: hrv-69 | Len: 2255 | Check: 7959 | Weight: 1.00 |
| Name: dq473489-70 | Len: 2255 | Check: 7471 | Weight: 1.00 |
| Name: hrv-72 | Len: 2255 | Check: 1505 | Weight: 1.00 |
| Name: hrv-79 | Len: 2255 | Check: 2821 | Weight: 1.00 |
| Name: hrv-83 | Len: 2255 | Check: 4330 | Weight: 1.00 |
| Name: hrv-84 | Len: 2255 | Check: 6613 | Weight: 1.00 |
| Name: hrv-86 | Len: 2255 | Check: 3381 | Weight: 1.00 |
| Name: hrv-91 | Len: 2255 | Check: 8414 | Weight: 1.00 |
| Name: hrv-92 | Len: 2255 | Check: 2492 | Weight: 1.00 |
| Name: ef173425-93 | Len: 2255 | Check: 691 | Weight: 1.00 |
| Name: hrv-97 | Len: 2255 | Check: 981 | Weight: 1.00 |
| Name: hrv-99 | Len: 2255 | Check: 839 | Weight: 1.00 |
| // | | | |

| | |
|---|---|
| | 1 50 |
| annotation | M......... .......... .......... .......... .........H |
| af499637-cva13 | MGAQVSSQKV GAHENTNVAT GGSTVNYTTI NYYKDSASNA ASKQDFSQDP |
| af546702-cva21 | MGAQVSTQKT GAHENQNVAA NGSTINYTTI NYYKDSASNS ATRQDLSQDP |
| v01149-pv1m | MGAQVSSQKV GAHENSNRAY GGSTINYTTI NYYRDSASNA ASKQDFSQDP |
| d00239-01 | MGAQVSRQNV GTHSTQNSVS NGSSLNYFNI NYFKDAASSG ASRLDFSQDP |
| pico-01 | MGAQVSRQNV GTHSTQNSVS NGSSLNYFNI NYFKDAASSG ASRLDFSQDP |
| hrv-01 | MGAQVSRQNV GTHSTQNSVS NGSSLNYFNI NYFKDAASSG ASRLDFSQDP |
| x02316-02 | MGAQVSRQNV GTHSTQNSVS NGSSLNYFNI NYFKDAASNG ASKLEFTQDP |
| hrv-07 | MGAQVSRQNV GTHSTQNAVT NGSSLNYFNI NYFKDAASSG ASRLDFSQDP |
| dq473503-07 | MGAQVSRQNV GTHSTQNAVT NGSSLNYFNI NYFKDAASSG ASRLDFSQDP |
| hrv-08 | MGAQVSRQNV GTHSTQNAVS GGSSLNYFNI NYFKDAASSG ASRLDFSQDP |
| pico-09 | MGAQVSRQNV GTHSTQNSVT NGSSLNYFNI NYFKDAASSR ASKLEFSQDP |
| hrv-09 | MGAQVSRQNV GTHSTQNSVS NGSSLNYFNI NYFKDAASSG ASKLEFSQDP |
| hrv-09-f01 | MGAQVSRQNV GTHSTQNSVS NGSSLNYFNI NYFKDAASSG ASKLEFSQDP |
| hrv-09-f02 | MGAQVSRQNV GTHSTQNSVS NGSSLNYFNI NYFKDAASSG ASKLEFSQDP |
| dq473498-10 | MGAQVSRQNV GTHSTQNAVS NGSSLNYFNI NYFKDAASSG ASKLEFSQDP |
| hrv-10 | MGAQVSRQNV GTHSTQNAVS NGSSLNYFNI NYFKDAASSG ASKLEFSQDP |
| ef173414-11 | MGAQVSRQNV GTHSTQNSVS NGSSLNYFNI NYFKDAASSG ASKLEFSQDP |
| ef173415-12 | MGAQVSRQNV GTHSTQNSVS NGSSLNYFNI NYFKDAASSG ASRLDFSQDP |
| hrv-13 | MGAQVSRQNV GTHSTQNSVS NGSSLNYFNI NYFKDAASSG ASKLEFSQDP |
| hrv-13-f03 | MGAQVSRQNV GTHSTQNSVS NGSSLNYFNI NYFKDAASSG ASKLEFSQDP |
| dq473493-15 | MGAQVSRQNV GTHSTQNTVS NGSSLNYFNI NYFKDAASSG ASKLEFSQDP |
| l24917-16 | MGAQVSRQNV GTHSTQNMVS NGSSLNYFNI NYFKDAASSG ASRLDFSQDP |
| hrv-18 | MGAQVSRQNV GTHSTQNSVS NGSSLNYFNI NYFKDAASHG ASKLDFSQDP |
| hrv-19 | MGAQVSRQNV GTHSTQNSVS NGSSLNYFNI NYFKDAASSG ASKLEFSQDP |
| hrv-20 | MGAQVSRQNV GTHSTQNAVS NGSSLNYFNI NYFKDAASSG ASRLDFSQDP |
| hrv-21 | MGTQVSRQNV GTHSTQNSVS NGSSLNYFNI NYFKDAASSG ASKLEFSQDP |
| hrv-22 | MGAQVSRQNV GTHSTQNTVS NGSSLNFFNI NYFKDAASSG ASKLEFSQDP |
| dq473497-23 | MGAQVSRQNV GTHSTQNSVS NGSSLNYFNI NYFKDAASNG ASKLEFSQDP |
| hrv-24 | MGAQVSRQNV GTHSTQNSVS NGSSLNYFNI NYFKDAASSG ASKLEFSQDP |
| ef173416-24 | MGAQVSRQNV GTHSTQNSVS NGSSLNYFNI NYFKDAASSG ASKLEFSQDP |
| hrv-25 | MGAQVSRQNV GTHSTQNSVS NGSSLNYFNI NYFKDAASSG ASKLEFSQDP |
| dq473508-28 | MGAQVSRQNV GTHTTQNAVS NGSSLNYFNI NYFKDAASSG ASRLDFSQDP |
| hrv-29 | MGAQVSRQNV GTHSTQNSVS NGSSLNYFNI NYFKDAASSG ASKLEFSQDP |
| dq473512-30 | MGAQVSRQNV GTHSTQNSVS NGSSLNYFNI NYFKDAASNG ASKLDFTQDP |
| hrv-30 | MGAQVSRQNV GTHSTQNSVS NGSSLNYFNI NYFKDAASNG ASKLDFTQDP |
| hrv-31 | MGAQVSRQNV GTHSTQNMVS NGSSLNYFNI NYFKDAASSG ASRLDFSQDP |
| hrv-32 | MGAQVSRQNV GTHSTQNSVT NGSSLNYFNI NYFKDAASSG ASKLEFSQDP |
| hrv-33 | MGAQVSRQNV GTHSTQNAVS NGSSLNYFNI NYFKDAASSG ASKLEFSQDP |
| hrv-34 | MGAQVSRQNV GTHSTQNTVS NGSSLNYFNI NYFKDAASHG ASKLDFSQDP |
| dq473501-34 | MGAQVSRQNV GTHSTQNTVS NGSSLNYFNI NYFKDAASHG ASKLDFSQDP |
| dq473505-36 | MGAQVSRQNV GTHSTQNSVS NGSSLNYFNI NYFKDAASSG ASRLDFSQDP |
| hrv-38 | MGAQVSRQNV GTHSTQNSVS NGSSLNYFNI NYFKDAASSG ASKLEFSQDP |
| dq473495-38 | MGAQVSRQNV GTHSTQNSVS NGSSLNYFNI NYFKDAASSG ASKLEFSQDP |
| ay751783-39 | MGAQVSRQNV GTHSTQNSVS GGSSLNYFNI NYFKDAASSG ASKLEFSQDP |
| hrv-40 | MGAQVSRQNV GTHSTQNSVS NGSSLNYFNI NYFKDAASSG ASKLEFSQDP |
| dq473491-41 | MGAQVSRQNV GTHSTQNSVS NGSSLNYFNI NYFKDAASSG ASKLEFSQDP |
| hrv-43 | MGAQVSRQNV GTHSTQNTVS NGSSLNYFNI NYFKDAASSG ASKLEFSQDP |
| dq473499-44 | MGAQVSRQNV GTHSTQNSVS NGSSLNYFNI NYFKDAASSG ASKLEFSQDP |
| hrv-45 | MGAQVSRQNV GTHSTQNTVT GGSSLNYFNI NYFKDAASSG ASRLDFSQDP |
| dq473506-46 | MGAQVSRQNV GTHSTQNTVA NGSSLNYFNI NYFKDAASSG ASRLDFSQDP |
| hrv-47 | MGAQVSRQNV GTHSTQNMVS NGSSLNYFNI NYFKDAASSG ASKLEFSQDP |
| dq473496-49 | MGAQVSRQNV GTHSTQNSVS NGSSLNYFNI NYFKDAASNG ASKLEFTQDP |
| hrv-49-f04 | MGAQVSRQNV GTHSTQNSVS NGSSLNYFNI NYFKDAASNG ASKLEFTQDP |
| hrv-50 | MGAQVSRQNV GTHSTQNSVS NGSSLNYFNI NYFKDAASHG ASKLDFSQDP |
| hrv-51 | MGAQVSRQNV GTHSTQNTVA NGSSLNYFNI NYFKDAASSG ASRLDFSQDP |
| dq473507-53 | MGAQVSRQNV GTHSTQNAVT NGSSLNYFNI NYFKDAASSG ASRLDFSQDP |
| hrv-54 | MGAQVSRQNV GTHSTQNSVS NGSSLNYFNI NYFKDAASSG ASKLEFSQDP |
| hrv-54-f05 | MGAQVSRQNV GTHSTQNSVS NGSSLNYFNI NYFKDAASSG ASKLEFSQDP |
| dq473511-55 | MGAQVSRQNV GTHSTQNSVS NGSSLNYFNI NYFKDAASSG ASKLEFSQDP |
| hrv-56 | MGAQVSRQNV GTHSTQNTVS NGSSLNYFNI NYFKDAASSG ASKLEFSQDP |
| hrv-57 | MGAQVSRQNV GTHSTQNSVS NGSSLNYFNI NYFKDAASSG ASKLEFSQDP |
| hrv-58 | MGAQVSRQNV GTHSTQNSVS NGSSLNYFNI NYFKDAASSX ASRLDFSQDP |
| dq473500-59 | MGAQVSRQNV GTHSTQNSVS NGSSLNYFNI NYFKDAASSG ASKLEFSQDP |
| hrv-60 | MGAQVSRQNV GTHSTQNVVS SGSSLNYFNI NYFKDAASSG ASKLEFSQDP |
| hrv-61 | MGAQVSRQNV GTHSTQNSVS NGSSLNYFNI NYFKDAASSG ASKLEFSQDP |
| hrv-62 | MGAQVSRQNV GTHSTQNSVS NGSSLNYFNI NYFKDAASSG ASKLEFSQDP |
| hrv-63 | MGAQVSRQNV GTHSTQNSVS NGSSLNYFNI NYFKDAASSG ASKLEFSQDP |
| hrv-64 | MGAQVSRQNV GTHSTQNSVS NGSSLNFFNI NYFKDAASSG ASKLEFSQDP |
| ef173417-64 | MGAQVSRQNV GTHSTQNSVS NGSSLNFFNI NYFKDAASSG ASKLEFSQDP |
| hrv-65 | MGAQVSRQNV GTHSTQNTVA NGSSLNYFNI NYFKDAASNG ASRLDFSQDP |
| hrv-66 | MGAQVSRQNV GTHSTQNAVS NGSSLNYFNI NYFKDAASSG ASKLEFSQDP |
| hrv-67 | MGAQVSRQNV GTHSTQNSVS NGSSLNYFNI NYFKDAASSG ASKLEFSQDP |
| hrv-68 | MGAQVSRQNV GTHSTQNAVS NGSSLNYFNI NYFKDAASSG ASRLDFSQDP |
| hrv-71 | MGAQVSRQNV GTHSTQNTVA NGSSLNYFNI NYFKDAASNG ASRLDFSQDP |
| dq473492-73 | MGAQVSRQNV GTHSTQNSVT NGSSLNYFNI NYFKDAASSG ASKLEFSQDP |
| dq473494-74 | MGAQVSRQNV GTHSTQNSVS NGSSLNYFNI NYFKDAASSG ASKLEFSQDP |
| dq473510-75 | MGAQVSRQNV GTHSTQNAVS NGSSLNYFNI NYFKDAASSG ASKLEFSQDP |
| dq473502-76 | MGAQVSRQNV GTHSTQNAVS NGSSLNYFNI NYFKDAASSG ASKLEFSQDP |
| hrv-76 | MGAQVSRQNV GTHSTQNAVS NGSSLNYFNI NYFKDAASSG ASKLEFSQDP |
| hrv-77 | MGAQVSRQNV GTHSTQNMVS NGSSLNYFNI NYFKDAASSG ASKLEFSQDP |
| hrv-78 | MGAQVSRQNV GTHSTQNSVS NGSSLNYFNI NYFKDAASSG ASRLDFSQDP |
| ef173418-78 | MGAQVSRQNV GTHSTQNSVS NGSSLNYFNI NYFKDAASSG ASRLDFSQDP |
| hrv-80 | MGAQVSRQNV GTHSTQNAVT NGSSLNYFNI NYFKDAASSG ASRLDFSQDP |
| hrv-81 | MGAQVSRQNV GTHSTQNSVS NGSSLNYFNI NYFKDAASSG ASRLDFSQDP |
| hrv-81-f06 | MGAQVSRQNV GTHSTQNSVS NGSSLNYFNI NYFKDAASSG ASRLDFSQDP |
| hrv-81-f07 | MGAQVSRQNV GTHSTQNSVS NGSSLNYFNI NYFKDAASSG ASRLDFSQDP |
| hrv-82 | MGAQVSRQNV GTHSTQNMVS NGSSLNYFNI NYFKDAASSG ASKLEFSQDP |
| dq473509-82 | MGAQVSRQNV GTHSTQNMVS NGSSLNYFNI NYFKDAASSG ASKLEFSQDP |
| hrv-85 | MGAQVSRQNV GTHSTQNSVS NGSSLNYFNI NYFKDAASSG ASKLEFSQDP |
| pico-85 | MGAQVSRQNV GTHSTQNSVS NGSSLNYFNI NYFKDAASSG ASKLEFSQDP |
| dq473504-88 | MGAQVSRQNV GTHSTQNAVT NGSSLNYFNI NYFKDAASSG ASRLDFSQDP |
| a10937-89 | MGAQVSRQNV GTHSTQNSVS NGSSLNYFNI NYFKDAASSG ASRLDFSQDP |
| hrv-89 | MGAQVSRQNV GTHSTQNSVS NGSSLNYFNI NYFKDAASSG ASRLDFSQDP |
| hrv-89-f08 | MGAQVSRQNV GTHSTQNSVS NGSSLNYFNI NYFKDAASSG ASRLDFSQDP |
| hrv-89-f09 | MGAQVSRQNV GTHSTQNSVS NGSSLNYFNI NYFKDAASSG ASRLDFSQDP |
| m16248-89 | MGAQVSRQNV GTHSTQNSVS NGSSLNYFNI NYFKDAASSG ASRLDFSQDP |
| hrv-90 | MGAQVSRQNV GTHSTQNSVS NGSSLNYFNI NYFKDAASSG ASKLEFSQDP |
| hrv-94 | MGAQVSRQNV GTHSTQNSVS NGSSLNYFNI NYFKDAASSG ASKLEFSQDP |
| ef173419-94 | MGAQVSRQNV GTHSTQNSVS NGSSLNYFNI NYFKDAASSG ASKLEFSQDP |
| hrv-95 | MGAQVSRQNV GTHSTQNAVS GGSSLNYFNI NYFKDAASSG ASRLDFSQDP |
| hrv-96 | MGAQVSRQNV GTHSTQNSVS NGSSLNYFNI NYFKDAASSG ASKLEFSQDP |
| hrv-98 | MGAQVSRQNV GTHSTQNSVS NGSSLNYFNI NYFKDAASSG ASKLEFSQDP |
| hrv-100 | MGAQVSRQNV GTHSTQNTVS NGSSLNYFNI NYFKDAASSG ASKLEFSQDP |
| ef186077-qpm | MGAQVSRQSV GSHETMIHAG TGAVVKYFNV NYYKDAASSG LTKQDFSQDP |
| ef077279-nat001 | MGAQVSRQSV GSHETMIHAG TGAVVKYFNV NYYKDAASSG LSKQDFSQDP |
| ny1078 | MGAQVSKQSV GAHETMVHAG SGAVVKYFNI NYYKDAASSG LTKQDFSQDP |
| ef582385-c024 | MGAQVSKQNV GSHENSVSAT GGSVIKYFNI NYYKDSASSG LTKQDFSQDP |
| ef077280-nat045 | MGAQVSKQNV GSHENSVSAS GGSVIKYFNI NYYKDSASSG LTKQDFSQDP |
| ef582387-c026 | MGAQVSRQSV GSHETMIHAG TGAVVKYFNV NYYKDAASSG LTKQDFSQDP |
| ef582386-c025 | MGAQVSKQNV GSHESGISAS SGSVIKYFNI NYYKDSASSG LSKQDFSMDP |
| dq473485-03 | MGAQVSTQKS GSHENQNILT NGSNQTFTVI NYYKDAASSS SAGQSFSMDP |
| ef173422-03 | MGAQVSTQKS GSHENQNILT NGSNQTFTVI NYYKDAASSS SAGQSFSMDP |
| dq473490-04 | MGAQVSTQKS GSHENQNILT NGSNQTFTVI NYYKDAASSS SAGQSLSMDP |
| hrv-05 | MGAQVSTQKS GSHENQNILT NGSNQTFTVI NYYKDAASSS SAGQSFSMDP |
| dq473486-06 | MGAQVSTQKS GSHENQNILT NGSNQTFTVI NYYKDAASSS SAGQSFSTDP |
| l05355-14 | MGAQVSTQKS GSHENQNILT NGSNQTFTVI NYYKDAASTS SAGQSLSMDP |
| k02121-14 | MGAQVSTQKS GSHENQNILT NGSNQTFTVI NYYKDAASTS SAGQSLSMDP |
| ef173420-17 | MGAQVSTQKS GSHENQNILT NGSTQHFTVI NYYKDAASSS SAGQSFSMDP |
| hrv-26 | MGAQVSTQKS GSHENQNFLS NGSNQTFTVI NYYKDAASSS SAGQSLSMDP |
| hrv-27 | MGAQVSTQKS GSHENQNILT NGSNQTFTVI NYYKDAASSS SAGQSFSMDP |
| ef173421-27 | MGAQVSTQKS GSHENQNILT NGSNQTFTVI NYYKDAASSS SAGQSFSMDP |
| dq473487-35 | MGAQVSTQKS GSHENQNILT NGSHQTFTVI NYYKDAASSS SASQSFSMDP |
| hrv-35 | MGAQVSTQKS GSHENQNILT NGSHQTFTVI NYYKDAASSS SASQSFSMDP |
| ef173423-37 | MGAQVSTQKS GSHENQNILT NGSNQTFTVI NYYKDAASSS SASQSFSMDP |
| hrv-42 | MGAQVSTQKS GSHENQNILT NGSNQTFTVI NYYKDAASSS SAGQSLSMDP |
| dq473488-48 | MGAQVSTQKT GSHENQNILT NGSTQHFTVI NYYKDAASSS SAGQSFSMDP |
| hrv-52 | MGAQVSTQKS GSHENQNILT NGSTQNFTVI NYYKDAASSS SAGQSFSMDP |
| hrv-52-f10 | MGAQVSTQKS GSHENQNILT NGSTQNFTVI NYYKDAASSS SAGQSFSMDP |
| ef173424-52 | MGAQVSTQKS GSHENQNILT NGSTQNFTVI NYYKDAASSS SAGQSFSMDP |
| hrv-69 | MGAQVSTQKS GSHENQNILT NGSTQHFTVI NYYKDAASSS SAGQSFSMDP |
| dq473489-70 | MGAQVSTQRS GSHENQNILT NGSTQHFTVI NYYKDAASSS SAGQSFSMDP |
| hrv-72 | MGAQVSTQKS GSHENQNILT NGSNQTFTVI NYYKDAASSS SAGQSLSMDP |
| hrv-79 | MGAQVSTQKS GSHENQNILT NGSHQTFTVI NYYKDAASSS SAGQSLSMDP |
| hrv-83 | MGAQVSTQKS GSHENQNILT NGSHQTFTVI NYYKDAASSS SAGQSLSMDP |
| hrv-84 | MGAQVSTQKS GSHENQNFLT NGSNQTFTVI NYYKDAASSS SAGQSLSMDP |
| hrv-86 | MGAQVSTQRS GSHENQNILT NGSHQTFTVI NYYKDAASAS SAGQSFSMDP |
| hrv-91 | MGAQVSTQKS GSHENQNILT NGSTQHFTVI NYYKDAASSS SASQSFSMDP |
| hrv-92 | MGAQVSTQKS GSHENQNILT NGSHQTFTVI NYYKDAASSS SAGQSLSMDP |
| ef173425-93 | MGAQISTQKS GSHENQNILT NGSNQTFTVI NYYKDAASSS SAGQSLSMDP |
| hrv-97 | MGAQVSTQKS GSHENQNILT NGSHQTFTVI NYYKDAASSS SAGQSLSMDP |
| hrv-99 | MGAQVSTQKS GSHENQNILT NGSNQTFTVI NYYKDAASSS SAGQSLSMDP |
| | 51 100 |
| annotation | HHHHH..... ........QS .......... ........BB BBB....... |
| af499637-cva13 | SKFTEPVKDV LIKSAPALNS PNIEACGYSD RVMQLTLGNS TITTQEAANS |
| af546702-cva21 | SKFTEPVKDL MLKTAPALNS PNVEACGYSD RVRQITLGNS TITTQEAANA |
| v01149-pv1m | SKFTEPIKDV LIKTAPMLNS PNIEACGYSD RVLQLTLGNS TITTQEAANS |
| d00239-01 | SKFTDPVKDV LEKGIPTLQS PSVEACGYSD RIIQITRGDS TITSQDVANA |
| pico-01 | SKFTDPVKDV LEKGIPTLQS PSVEACGYSD RIMQITRGDS TITSQDVANA |
| hrv-01 | SKFTDPVKDV LEKGIPTLQS PSVEACGYSD RIMQITRGDS TITSQDVANA |
| x02316-02 | SKFTDPVKDV LEKGIPTLQS PTVEACGYSD RIIQITRGDS TITSQDVANA |
| hrv-07 | SKFTDPVKDV LEKGIPTLQS PTVEACGYSD RLMQITRGDS TITSQDIANA |
| dq473503-07 | SKFTDPVKDV LEKGIPTLQS PTVEACGYSD RLMQITRGDS TITSQDIANA |
| hrv-08 | SKFTDPVKDV LTKGIPTLQS PTVEACGYSD RIIQITRGDT TITSQDIANA |
| pico-09 | SKFTDPVKSV LEKGIPTLQS PTVEACGYSD RIIQITRGDS TITSQDVANA |
| hrv-09 | SKFTDPVKDV LEKGIPTLQS PTVEACGYSD RIIQITRGDS TITSQDVANA |
| hrv-09-f01 | SKFTDPVKDV LEKGIPTLQS PTVEACGYSD RIIQITRGDS TITSQDVANA |
| hrv-09-f02 | SKFTDPVKDV LEKGIPTLQS PTVEACGYSD RIIQITRGDS TITSQDVANA |
| dq473498-10 | SKFTDPVKDV LEKGIPTLQS PTVEACGYSD RIIQITRGDS TITSQDVANA |
| hrv-10 | SKFTDPVKDV LEKGIPTLQS PTVEACGYSD RIIQITRGDS TITSQDVANA |
| ef173414-11 | SKFTDPVKDV LEKGIPTLQS PTVEACGYSD RIIQITRGDS TITSQDVANA |
| ef173415-12 | SKFTDPVKDV LTKGIPTLQS PTVEACGYSD RIIQITRGDS TITSQDIANA |
| hrv-13 | SKFTDPVKDV LEKGIPTLQS PTVEACGYSD RIIQITRGDS TITSQDVANA |
| hrv-13-f03 | SKFTDPVKDV LEKGIPTLQS PTVEACGYSD RIIQITRGDS TITSQDVANA |
| dq473493-15 | SKFTDPVKDV LEKGIPTLQS PTVEACGYSD RIIQITRGDS TITSQDVANA |
| l24917-16 | SKFTDPVKDV LEKGIPTLQS PSVEACGYSD RIIQITRGDS TITSQDVANA |
| hrv-18 | SKFTDPVKDV LEKGIPTLQS PTVEACGYSD RIIQITRGDS TITSQDVANA |
| hrv-19 | SKFTDPVKDV LEKGIPTLQS PTVEACGYSD RIIQITRGDS TITSQXVANA |
| hrv-20 | SKFTDPVKDV LLKDIPTLQS PSVEACGYSD RIIQITRGNS TITSQDIANA |
| hrv-21 | SKFTDPVKDV LEKGIPTLQS PTVEACGYSD RIIQITRGDS TITSQDVANA |
| hrv-22 | SKFTDPVKDV LEKGIPTLQS PTVEACGYSD RIIQITRGDS TITSQDVANA |
| dq473497-23 | SKFTDPVKDV LEKGIPTLQS PTVEACGYSD RIIQITRGDS TITSQDVANA |
| hrv-24 | SKFTDPVKDV LEKGIPTLQS PTVEACGYSD RIMQITRGDS TITSQDVANA |
| ef173416-24 | SKFTDPVKDV LEKGIPTLQS PTVEACGYSD RIMQITRGDS TITSQDVANA |
| hrv-25 | SKFTDPVKDV LEKGIPTLQS PTVEACGYSD RIMQITRGDS TITSQDVANA |
| dq473508-28 | SKFTDPVKDV LTKGIPTLQS PSVEACGYSD RIMQITRGNS TITSQDVANA |
| hrv-29 | SKFTDPVKDV LEKGIPTLQS PTVEACGYSD RIMQITRGDS TITSQDVANA |
| dq473512-30 | SKFTDPVKDV LEKGIPTLQS PTVEACGYSD RIIQITRGDS TITSQDVANA |
| hrv-30 | SKFTDPVKDV LEKGIPTLQS PTVEACGYSD RIIQITRGDS TITSQDVANA |
| hrv-31 | SKFTDPVKDV LEKGIPTLQS PTVEACGYSD RIMQITRGDS TITSQDVANA |
| hrv-32 | SKFTDPVKDV LEKGIPTLQS PTVEACGYSD RIIQITRGDS TITSQDVANA |
| hrv-33 | SKFTDPVKDV LEKGIPTLQS PTVEACGYSD RIIQITRGDS TITSQDVANA |
| hrv-34 | SKFTDPVKDV LEKGIPTLQS PTVEACGYSD RIIQITRGDS TITSQDVANA |
| dq473501-34 | SKFTDPVKDV LEKGIPTLQS PTVEACGYSD RIIQITRGDS TITSQDVANA |
| dq473505-36 | SKFTDPVKDV LEKGIPTLQS PTVEACGYSD RLIQITRGDS TITSQDIANA |
| hrv-38 | SKFTDPVKDV LEKGIPTLQS PTVEACGYSD RIIQITRGDS TITSQDVANA |
| dq473495-38 | SKFTDPVKDV LEKGIPTLQS PTVEACGYSD RIIQITRGDS TITSQDVANA |
| ay751783-39 | SKFTDPVKDV LEKGIPTLQS PTVEACGYSD RIIQITRGDS TITSQDVANA |
| hrv-40 | SKFTDPVKDV LEKGIPTLQS PTVEACGYSD RIIQITRGDS TITSQDVANA |
| dq473491-41 | SKFTDPVKDV LEKGIPTLQS PTVEACGYSD RIIQITRGDS TITSQDVANA |
| hrv-43 | SKFTDPVKDV LEKGIPTLQS PTVEACGYSD RIIQITRGDS TITSQDVANA |
| dq473499-44 | SKFTDPVKDV LEKGIPTLQS PTVEACGYSD RIMQITRGDS TITSQDVANA |
| hrv-45 | SKFTDPVKDV LTKGIPTLQS PTVEACGYSD RIIQITRGDT TITSQDIANA |
| dq473506-46 | SKFTDPVKDV LTKGIPTLQS PSVEACGYSD RIIQITRGNS TITSQDIANA |
| hrv-47 | SKFTDPVKDV LEKGIPTLQS PTVEACGYSD RIMQITRGDS TITSQDVANA |
| dq473496-49 | SKFTDPVKDV LEKGIPTLQS PTVEACGYSD RIIQITRGDS TITSQDVANA |
| hrv-49-f04 | SKFTDPVKDV LEKGIPTLQS PTVEACGYSD RIIQITRGDS TITSQDVANA |
| hrv-50 | SKFTDPVKDV LEKGIPTLQS PTVEACGYSD RIIQITRGDS TITSQDVANA |
| hrv-51 | SKFTDPVKDV LIKGVPTLQS PTVEACGYSD RIIQITRGNS TITSQDIANA |
| dq473507-53 | SKFTDPVKDV LTKGIPTLQS PSVEACGYSD RIIQITRGNS TITSQDVANA |
| hrv-54 | SKFTDPVKDV LEKGIPTLQS PTVEACGYSD RIIQITRGDS TMTSQDVANA |
| hrv-54-f05 | SKFTDPVKDV LEKGIPTLQS PTVEACGYSD RIIQITRGDS TITSQDVANA |
| dq473511-55 | SKFTDPVKDV LEKGIPTLQS PSVEACGYSD RIIQITRGDS TITSQDVANA |
| hrv-56 | SKFTDPVKDV LEKGIPTLQS PTVEACGYSD RIIQITRGDS TITSQDVANA |
| hrv-57 | SKFTDPVKDV LEKGIPTLQS PTVEACGYSD RIIQITRGDS TITSQDVANA |
| hrv-58 | SKFTDPVKDV LEKGIPTLQS PTVEACGYSD RLIQITRGDS TITSQDIANA |
| dq473500-59 | SKFTDPVKDV LEKGIPTLQS PTVEACGYSD RIIQITRGDS TITSQDVANA |
| hrv-60 | SKFTDPVKDV LEKGIPTLQS PTVEACGYSD RIIQITRGDS TIISQDVANA |
| hrv-61 | SKFTDPVKDV LEKGIPTLQS PTVEACGYSD RIIQITRGDS TITSQDVANA |
| hrv-62 | SKFTDPVKDV LEKGIPTLQS PTVEACGYSD RIIQITRGDS TITSQDVANA |
| hrv-63 | SKFTDPVKDV LEKGIPTLQS PTVEACGYSD RIIQITRGDS TITSQDVANA |
| hrv-64 | SKFTDPVKDV LEKGIPTLQS PTVEACGYSD RIIQITRGDS TITSQDVANA |
| ef173417-64 | SKFTDPVKDV LEKGIPTLQS PTVEACGYSD RIIQITRGDS TITSQDVANA |
| hrv-65 | SKFTDPVKDV LTKGIPTLQS PSVEACGYSD RIIQITRGNS TITSQDIANA |
| hrv-66 | SKFTDPVKDV LXKGIPTLQS PTVEACGYSD RIIQITRGDS TITSQDVANA |
| hrv-67 | SKFTDPVKDV LEKGIPTLQS PTVEACGYSD RIIQITRGDS TITSQDVANA |
| hrv-68 | SKFTDPVKDV LMKDIPTLQS PSVEACGYSD RIIQITRGNS TITSQDIANA |
| hrv-71 | SKFTDPVKDV LIKGVPTLQS PSVEACGYSD RIIQITRGNS TITSQDIANA |
| dq473492-73 | SKFTDPVKDV LEKGIPTLQS PTVEACGYSD RIIQITRGDS TITSQDVANA |
| dq473494-74 | SKFTDPVKDV LEKGIPTLQS PTVEACGYSD RIIQITRGDS TITSQDVANA |
| dq473510-75 | SKFTDPVKDV LEKGIPTLQS PTVEACGYSD RIIQITRGDS TITSQDVANA |
| dq473502-76 | SKFTDPVKDV LEKGIPTLQS PTVEACGYSD RIIQITRGDS TITSQDVANA |
| hrv-76 | SKFTDPVKDV LEKGIPTLQS PTVEACGYSD RIIQITRGDS TITSQDVANA |
| hrv-77 | SKFTDPVKDV LEKGIPTLQS PTVEACGYSD RIIQITRGDS TITSQDVANA |
| hrv-78 | SKFTDPVKDV LTKGVPTLQS PSVEACGYSD RIIQITRGDS TITSQDIANA |
| ef173418-78 | SKFTDPVKDV LTKGVPTLQS PSVEACGYSD RIIQITRGDS TITSQDIANA |
| hrv-80 | SKFTDPVKDV LTKGIPTLQS PSVEACGYSD RIIQITRGNS TITSQDIANA |
| hrv-81 | SKFTDPVKDV LEKGIPTLQS PSVEACGYSD RIIQITRGDS TITSQDVANA |
| hrv-81-f06 | SKFTDPVKDV LEKGIPTLQS PSVEACGYSD RIIQITRGDS TITSQDVANA |
| hrv-81-f07 | SKFTDPVKDV LEKGIPTLQS PSVEACGYSD RIIQITRGDS TITSQDVANA |
| hrv-82 | SKFTDPVKDV LEKGIPTLQS PTVEACGYSD RIIQITRGDS TITSQDVANA |
| dq473509-82 | SKFTDPVKDV LEKGIPTLQS PTVEACGYSD RIIQITRGDS TITSQDVANA |
| hrv-85 | SKFTDPVKDV LEKGIPTLQS PTVEACGYSD RIIQITRGDS TITSQDVANA |
| pico-85 | SKFTDPVKDV LEKGIPTLQS PTVEACGYSD RIIQITRGDS TITSQDVANA |
| dq473504-88 | SKFTDPVKDV LEKGIPTLQS PSVEACGYSD RLMQITRGDS TITSQDIANA |
| a10937-89 | SKFTDPVKDV LEKGIPTLQS PTVEACGYSD RLIQITRGDS TITSQDTANA |
| hrv-89 | SKFTDPVKDV LEKGIPTLQS PTVEACGYSD RLIQITRGDS TITSQDIANA |
| hrv-89-f08 | SKFTDPVKDV LEKGIPTLQS PTVEACGYSD RLIQITRGDS TITSQDIANA |
| hrv-89-f09 | SKFTDPVKDV LEKGIPTLQS PTVEACGYSD RLIQITRGDS TITSQDIANA |
| m16248-89 | SKFTDPVKDV LEKGIPTLQS PTVEACGYSD RLIQITRGDS TITSQDTANA |
| hrv-90 | SKFTDPVKDV LEKGIPTLQS PSVEACGYSD RIIQITRGDS TITSQDVANA |
| hrv-94 | SKFTDPVKDV LEKGIPTLQS PTVEACGYSD RIIQITRGDS TITSQDVANA |
| ef173419-94 | SKFTDPVKDV LEKGIPTLQS PTVEACGYSD RIIQITRGDS TITSQDVANA |
| hrv-95 | SKFTDPVKDV LTKGIPTLQS PTVEACGYSD RIIQITRGDT TITSQDIANA |
| hrv-96 | SKFTDPVKDV LEKGIPTLQS PTVEACGYSD RIIQITRGDS TITSQDVANA |
| hrv-98 | SKFTDPVKDV LEKGIPTLQS PTVEACGYSD RIIQITRGDS TITSQDVANA |
| hrv-100 | SKFTDPVKDV LEKGIPTLQS PTVEACGYSD RIIQITRGDS TITSQDVANA |
| ef186077-qpm | SKFTQPVADI LTN..PALMS PSIEACGFSD RLKQITIGNS TITTQDAVNT |
| ef077279-nat001 | SKFTQPVADV LTN..PALMS PSVEACGFSD RLKQITIGNS TITTQDAVNT |
| ny1078 | SKFTQPVADL LTN..PALMS PSVEACGYSD RLKQITIGSS TITTQDSVNT |
| ef582385-c024 | SKFTQPLAEA LTN..PALMS PTVEACGMSD RLKQITIGNS TITTQDTLNS |
| ef077280-nat045 | SKFTQPIADV LTN..PALMS PTVEACGFSD RLKQITIGNS TITTQDSLNT |
| ef582387-c026 | SKFTQPVADI LSN..PALMS PSIEACGFSD RLKQITIGNS TITTQDAVNT |
| ef582386-c025 | EKFTKPLADV MTN..PALMS PSIEACGFSD RLKQITIGSS TITTQDTLNT |
| dq473485-03 | SKFTEPVKDL MLKGAPALNS PNVEACGYSD RVQQITLGNS TITTQEAANA |
| ef173422-03 | SKFTEPVKDL MLKGAPALNS PNVEACGYSD RVQQITMGNS TITTQEAANA |
| dq473490-04 | SKFTEPVKDL MLKGAPALNS PNIEACGYSD RVEQITLGNS TITTQEAANS |
| hrv-05 | SKFTEPVKDI MLKGAPALNS PNIEACGYSD RVEQITLGNS TITTQEAANS |
| dq473486-06 | SKFTEPVKDL MLKGAPALNS PNVEACGYSD RVQQITLGNS TITTQEAANA |
| l05355-14 | SKFTEPVKDL MLKGAPALNS PNVEACGYSD RVQQITLGNS TITTQEAANA |
| k02121-14 | SKFTEPVKDL MLKGAPALNS PNVEACGYSD RVQQITLGNS TITTQEAANA |
| ef173420-17 | SKFTEPVKDI MLKGAPALNS PNVEACGYSD RVQQITLGNS TITTQEAANA |
| hrv-26 | SKFTEPVKDL MLKGAPALNS PNIEACGYSD RVEQITIGNS TITTQEAANT |
| hrv-27 | SKFTEPVKDI MLKGAPALNS PNIEACGYSD RVEQITIGNS TITTQEAANA |
| ef173421-27 | SKFTEPVKDI MLKGAPALNS PNIEACGYSD RVEQITIGNS TITTQEAANA |
| dq473487-35 | SKSTEPVKDL MLKGAPALNS PNVEACGYSD RVQQITLGNS TITTQEAANA |
| hrv-35 | SKFTEPVKDL MLKGAPALNS PNVEACGYSD RVQQITLGNS TITTQEAANA |
| ef173423-37 | SKFTEPVKDL MLKGAPALNS PNVEACGYSD RVQQITLGNS TITTQEAANA |
| hrv-42 | SKFTEPVKDI MLKGAPALNS PNIEACGYSD RVEQITLGNS TITTQEAANS |
| dq473488-48 | SKFTEPVKDI MIKGAPALNS PNVEACGYSD RVQQITLGNS TITTQEAANA |
| hrv-52 | SKFTEPVKDI MLKGAPALNS PNVEACGYSD RVQQITLGNS TITTQEAANA |
| hrv-52-f10 | SKFTEPVKDI MLKGAPALNS PNVEACGYSD RVQQITLGNS TITTQEAANA |
| ef173424-52 | SKFTEPVKDI MLKGAPALNS PNVEACGYSD RVQQITLGNS TITTQEAANA |
| hrv-69 | SKFTEPVKDI MLKGAPALNS PNVEACGYSD RVQQITLGNS TITTQEAANA |
| dq473489-70 | SKFTEPVKDI MLKGAPALNS PNVEACGYSD RVQQITLGNS TITTQEAANA |
| hrv-72 | SKFTEPVKDL MLKGAPALNS PNVEACGYSD RVQQITLGNS TITTQEAANA |
| hrv-79 | SKFTEPVKDL MLKGAPALNS PNVEACGYSD RVQQITLGNS TITTQEAANA |
| hrv-83 | SRFTEPVKDL MLKGAPALNS PNVEACGYSD RVQQITLGNS TITTQEAANA |
| hrv-84 | SKFTEPVKDL MLKGAPALNS PNIEACGYSD RVEQITIGNS TITTQEAANA |
| hrv-86 | SKFTEPVKDL MLKGAPALNS PNVEACGYSD RVQQITLGNS TITTQEAANA |
| hrv-91 | SKFTEPVKDI MLKGAPALNS PNVEACGYSD RVQQITLGNS TITTQEAANA |
| hrv-92 | SKFTEPVKDL MLKGAPALNS PNVEACGYSD RVQQITLGNS TITTQEAANA |
| ef173425-93 | SKFTEPVKDI MLKGAPALNS PNIEACGYSD RVEQITIGNS TITTQEAANA |
| hrv-97 | SKFTEPVKDI MLKGAPALNS PNVEACGYSD RVEQITIGNS TITTQEAANA |
| hrv-99 | SKFTEPVKDI MLKGAPALNS PNIEACGYSD RVEQITLGNS TITTQEAANT |
| | 101 150 |
| annotation | .......... .......... ....HHHZHH ..BBBBBBBB .....BBCBB |
| af499637-cva13 | VVAYGVWPSY LSDKDANPVD QPTEPDVSAC RFYTLDTVEW DKESKGWWWK |
| af546702-cva21 | IVAYGEWPTY INDSEANPVD APTEPDVSSN RFYTLESVSW KTTSRGWWWK |
| v01149-pv1m | VVAYGRWPEY LRDSEANPVD QPTEPDVAAC RFYTLDTVSW TKESRGWWWK |
| d00239-01 | VVGYGVWPHY LTPQDATAID KPTQPDTSSN RFYTLESKHW NGDSKGWWWK |
| pico-01 | VVGYGVWPHY LTPQDATAID KPTQPDTSSN RFYTLESKHW NGSSKGWWWK |
| hrv-01 | VVGYGVWPHY LTPQDATAID KPTQPDTSSN RFYTLESKHW NGSSKGWWWK |
| x02316-02 | IVAYGVWPHY LSSKDASAID KPSQPDTSSN RFYTLRSVTW SSSSKGWWWK |
| hrv-07 | VVGYGVWPSY LTPDDATAID KPTQPDTSSN RFYTLDSRTW TSASLGWWWK |
| dq473503-07 | VVGYGVWPSY LTPDDATAID KPTQPDTSSN RFYTLDSRTW TSASLGWWWK |
| hrv-08 | VVAYGVWPTY LNSKDATAID KPTQPDTSTN RFYTLESREW RSDSKGWWWK |
| pico-09 | VVGYGVWPHY LTPQDATAID KPSHPDTSSN RFYTLESKTW SGDSKGWWWK |
| hrv-09 | IVGYGVWPHY LTPQDATAIX KPSHPDTSSN RFYTLESKTW SSDSKGWWWK |
| hrv-09-f01 | VVGYGVWPHY LTPQDATAID KPSHPDTSSN RFYTLESKTW SNESKGWWWK |
| hrv-09-f02 | VVGYGVWPHY LTPQDATAID KPSHPDTSSN RFYTLESKTW SNESKGWWWK |
| dq473498-10 | VVGYGVWPHY LSATDATAID KPTQPDTSSN RFYTLESKVW TSTSQGWWWK |
| hrv-10 | VVGYGVWPHY LSATDATAID KPTQPDTSSN RFYTLESKVW TSTSQGWWWK |
| ef173414-11 | VVGYGVWPHY LTSQDATAID KPSRPDTSSN RFYTLESKHW TSTSKGWWWK |
| ef173415-12 | VVAYGVWPHY LSAEDANAID KPTRPDTSSN RFYTLESKQW MENSKGWWWK |
| hrv-13 | VVGYGVWPHY LTPQDATAID KPTRPDTSSN RFYTLKSQLW TAESKGWWWK |
| hrv-13-f03 | VVGYGVWPHY LTPQDATAID KPTRPDTSSN RFYTLESQLW TAESKGWWWK |
| dq473493-15 | VVGYGVWPHY LTPEDATAID KPSHPDTSSN RFYTLDSKLW SGDSKGWWWK |
| l24917-16 | VVGYGVWPHY LTPQDATAID KPTQPDTSSN RFYTLDSKMW NSTSKGWWWK |
| hrv-18 | VVGYGVWPHY LTPQDATAID KPSRPDTSSN RFYTLESKTW DSSSKGWWWK |
| hrv-19 | VVGYGVWPHY LTPEDATAID KPSQPDTSTN RFYTLESKVW DSESKGWWWK |
| hrv-20 | VVAYGVWPQY LTPDHASAID KPTHPDISSN RFYTLESKEW TRDSKGWWWK |
| hrv-21 | VVGYGVWPHY LTPQDATAID KPSRPDTSSN RFYTLESKMW TSDSKGWWWK |
| hrv-22 | VVGYGVWPHY LTPEDATAID KPTQPDTSSN RFYTLESKIW ASNSKGWWWK |
| dq473497-23 | VVAYGVWPHY LSSEDATAID KPSRPDTSSN RFYTLKSITW SRSSKGWWWK |
| hrv-24 | VVAYGVWPHY LTPQDATAID KPSRPDTSSN RFYTLESKTW TGTSKGWWWK |
| ef173416-24 | VVAYGVWPHY LTPQDATAID KPSRPDTSSN RFYTLESKTW TGTSKGWWWK |
| hrv-25 | VIGYGIWPHY LSAEDATAID KPTQPDTSSN RFYTLESKTW HADSKGWWWK |
| dq473508-28 | VVAYGVWPEY LPSQDASAID KPTHPDTSSN RFYTLESKNW TETSKGWWWK |
| hrv-29 | VIGYGVWPHY LSAEDATAID KPTQPDTSSN RFYTLESKTW NRQSKGWWWK |
| dq473512-30 | VVAYGVWPHY LSSKDATAID KPSRLDTSSN RFYTLKSIIW SGASKGWWWK |
| hrv-30 | VVAYGVWPHY LSSKDATAID KPSRPDTSSN RFYTLKSIIW SGASKGWWWK |
| hrv-31 | VVGYGIWPHY LSAKDATAID KPTQPDTSSN RFYTLESKIW TRDSKGWWWK |
| hrv-32 | VVAYGVWPHY LTPQDATAID KPSHPDTSSN RFYTLESKNW DSESKGWWWK |
| hrv-33 | VVGYGVWPHY LTPQDATAID KPSRPDTSSN RFYTLASKEW DFNSKGWWWK |
| hrv-34 | VVGYGVWPHY LTPQDATAID KPSRPDTSSN RFYTLESKMW DSSSKGWWWK |
| dq473501-34 | VVGYGVWPHY LTPQDATAID KPSRPDTSSN RFYTLESKMW DSSSKGWWWK |
| dq473505-36 | VVGYGVWPSY LTPDDATAID KPTQPDTSSN RFYTLESRSW TSSSSGWWWK |
| hrv-38 | VVGYGVWPHY LTSRDATAID KPSQPDTSSN RFYTLESKTW TSDSKGWWWK |
| dq473495-38 | VVGYGVWPHY LTSRDATAID KPSQPDTSSN RFYTLESKTW TSDSKGWWWK |
| ay751783-39 | VVGYGVWPHY LTADDASAID KPTQPDTSSN RFYTLESKVW KRDSKGWWWK |
| hrv-40 | VVGYGVWPHY LTPQDATAID KPSQPDTSSN RFYTLESKEW TNSSQGWWWK |
| dq473491-41 | VVGYGVWPHY LTSQDATAID KPTRPDTSSN RFYTLESQLW TNTSKGWWWK |
| hrv-43 | VVAYGVWPHY LTPQDATAID KPSQPETSSD RFYTLESRSW DSTSKGWWWK |
| dq473499-44 | VIGYGVWPHY LSAEDATAID KPTQPDTSSN RFYTLESKTW DRQSKGWWWK |
| hrv-45 | VVGYGVWPHY LDSKDASAID KPTQPDTSAN RFYTLESKEW TPDSKGWWWK |
| dq473506-46 | VVGYGVWPEY LTDDDASAID KPTHPDTSSN RFYTLESKHW ENNSKGWWWK |
| hrv-47 | VVGYGVWPHY LSAKDATAID KPTQPDTSSN RFYTLESKIW KPDSKGWWWK |
| dq473496-49 | VVAYGVWPHY LSSKDASAID KPSRPDTSSN RFYTLKSVTW TNSSKGWWWK |
| hrv-49-f04 | VVAYGVWPHY LSSKDASAID KPSRPDTSSN RFYTLKSVTW SNSSKGWWWK |
| hrv-50 | VVGYGVWPHY LSSQDATAID KPSRPDTSSN RFYTLESKSW DSMSKGWWWK |
| hrv-51 | VVAYGVWPHY LSDTDANAID KPSHPDTSSD RFYTLDSKLW SNESKGWWWK |
| dq473507-53 | VVAYGVWPEY LSAQDASAID KPSHPDTSSN RFYTLESKNW TNTSKGWWWK |
| hrv-54 | VVGYGVWPHY LTAQDATAID KPTQPDTSSN RFYTLESKLW DETSKGWWWK |
| hrv-54-f05 | VVGYGVWPHY LTAQDATAID KPTQPDTSSN RFYTLDSKLW DETSKGWWWK |
| dq473511-55 | VVGYGVWPHY LTPQDATALD KPSRPDTSSN RFYTLESKIW NGTSKGWWWK |
| hrv-56 | VVGYGVWPHY LTPQDATAID KPTQPDTSSN RFYTLESKVW TQTSKGWWWK |
| hrv-57 | VVGYGVWPHY LTPQDATAID KPSRPDTSSN RFYTLESKMW SSTSKGWWWK |
| hrv-58 | VVGYGVWPSY LTPDDATAID KPTQPDTSSN RFYTLESRNW TNSSTGWWWK |
| dq473500-59 | VVGYGVWPHY LTPQDATAID KPTQPDTSSN RFYTLESKTW VETSKGWWWK |
| hrv-60 | VVGYGVWPHY LTAQDATAID KPSQPDTSSN RFYTLESKLW TNESKGWWWK |
| hrv-61 | VVGYGVWPHY LTPQDATAID KPSKPDTSSN RFYTLESQTW TNESKGWWWK |
| hrv-62 | VIGYGIWPHY LSAEDATAID KPTQPDTSSN RFYTLESKIW HTNSKGWWWK |
| hrv-63 | VVGYGVWPQY LSSQDATAID KPTQPDTSSN RFYTLESKVW TRTSKGWWWK |
| hrv-64 | VVGYGVWPHY LTPQDATAID KPTQPDTSSN RFYTLESKTW ASNSKGWWWK |
| ef173417-64 | VVGYGVWPHY LTPQDATAID KPTQPDTSSN RFYTLESKTW ASNSKGWWWK |
| hrv-65 | VVGYGVWPQY LSDADANAID KPTHPDTSSN RFYTLDSKLW TSTSKGWWWK |
| hrv-66 | VVGYGVWPHY LSGADATAID KPSRPDTSSN RFYTLESKVW TPTSQGWWWK |
| hrv-67 | VVGYGVWPHY LTPQDATAID KPSHPDTSSN RFYTLESKTW SADSKGWWWK |
| hrv-68 | VVAYGVWPQY LSPDDASAID KPTHPDVSSN RFYTLDSKEW TNESKGWWWK |
| hrv-71 | VVAYGVWPQY LSDADANAID KPSHPDTSSN RFYTLDSKLW ENGSKGWWWK |
| dq473492-73 | VVAYGVWPHY LTPQDATAID KPTRPDTSSN RFYTLESQMW SPESKGWWWK |
| dq473494-74 | VVGYGVWPHY LTPEDATAID KPSHPDTSAN RFYTLESKLW NGESKGWWWK |
| dq473510-75 | VVAYGVWPHY LTPQDATAID KPSQPETSSN RFYTLESKTW DSSSKGWWWK |
| dq473502-76 | VVGYGVWPHY LTSQDATAID KPSRPDTSSN RFYTLESKEW SANSKGWWWK |
| hrv-76 | VVGYGVWPHY LTSQDATAID KPSRPDTSSN RFYTLESKEW SANSKGWWWK |
| hrv-77 | VVGYGVWPHY LSAADATAID KPTQPDTSSN RFYTLESKVW SSTSQGWWWK |
| hrv-78 | VVAYGVWPSY LTAKDASAID KPTQPDTSSN RFYTLESKNW QSDSKGWWWK |
| ef173418-78 | VVAYGVWPSY LTAKDASAID KPTQPDTSSN RFYTLESKNW QSDSKGWWWK |
| hrv-80 | VVAYGVWPEY LSSDDASAID KPTHPDTSSN RFYTLESKQW DTNSKGWWWK |
| hrv-81 | VVGYGVWPHY LNSRDAVAID KPTQPDTSSN RFYTLDSKMW DNNSKGWWWK |
| hrv-81-f06 | VVGYGVWPHY LNSRDAVAID KPTQPDTSSN RFYTLDSKMW DSNSKGWWWK |
| hrv-81-f07 | VVGYGVWPHY LNSRDAVAID KPTQPDTSSN RFYTLDSKMW DSNSKGWWWK |
| hrv-82 | VVGYGIWPHY LSPEDATAID KPTRPDTSSN RFYTLESKIW TSDSKGWWWK |
| dq473509-82 | VVGYGIWPHY LSPEDATAID KPTRPDTSSN RFYTLESKIW TRDSKGWWWK |
| hrv-85 | VVGYGVWPHY LTPQDATAID KPSQPDTSSN RFYTLESKEW TNSSQGWWWK |
| pico-85 | VVGYGVWPHY LTPQDATAID KPSQPDTSSN RFYTLESKEW TNSSQGWWWK |
| dq473504-88 | VVGYGVWPSY LTPDDATAID KPTQPDTSSN RFYTLESRTW TTASTGWWWK |
| a10937-89 | VVAYGVWPSY LTPDDATAID KPTQPDTSSN RFYTLDSRSW TSASSGWWWK |
| hrv-89 | VVAYGVWPSY LTPDDATAID KPTQPDTSSN RFYTLDSRSW TSASSGWWWK |
| hrv-89-f08 | VVAYGVWPSY LTPDDATAID KPTQPDTSSN RFYTLDSRSW TSASSGWWWK |
| hrv-89-f09 | VVAYGVWPSY LTPDDATAID KPTQPDTSSN RFYTLDSRSW TSASSGWWWK |
| m16248-89 | VVAYGVWPSY LTPDDATAID KPTQPDTSSN RFYTLDSRSW TSASSGWWWK |
| hrv-90 | VVAYGVWPHY LTPQDATAID KPSRPDTSSN RFYTLESKTW TGSSKGWWWK |
| hrv-94 | VVGYGVWPHY LTPQDATAID KPTQPDTSSN RFYTLESKIW TTDSKGWWWK |
| ef173419-94 | VVGYGVWPHY LTPQDATAID KPTQPDTSSN RFYTLESKIW TTDSKGWWWK |
| hrv-95 | VVAYGVWPTY LNSKDATAID KPTQPDTSTN RFYTLESREW RSDSKGWWWK |
| hrv-96 | VVGYGVWPHY LTPQDATAID KPSRPDTSSN RFYTLESQTW SGDSKGWWWK |
| hrv-98 | VVGYGVWPHY LTAQDATAID KPTQPDTSSN RFYTLESKLW HETSKGWWWK |
| hrv-100 | VVGYGVWPHY LSAADATAID KPTQPDTSSN RFYTLESKIW TGTSQGWWWK |
| ef186077-qpm | IVAYGEWPSY LSDIDATSVD KPTHPETSSD RFYTLKSVTW EVGSSGWWWK |
| ef077279-nat001 | IVAYGEWPSY LSDLDATSVD KPTHPETSSD RFYTLKSVEW QSSSEGWWWK |
| ny1078 | IVAYGEWPSY LSDLDASSVD KPTHPETSAD RFYTLDSVQW GGSSKGWWWK |
| ef582385-c024 | ILAYGEWPKY LSDLDASSVD KPTHPETSSD RFYTLTSVDW TTTSKGWWWK |
| ef077280-nat045 | IVAYGEWPEY LSDLDATSVD KPTHPETSSD RFYTLESVMW HGSSRGWWWK |
| ef582387-c026 | IVAYGEWPSY LSDMDATSVD KPTHPETSSD RFYTLNSVTW QGGSLGWWWK |
| ef582386-c025 | VVAYGEWPEY LKDTDASAVD KPTHPETSTD RFYTLTSVNG MASSKGWWWK |
| dq473485-03 | IVCYAEWPEY LSDNDASDVN KTSKPDISVC RFYTLDSKTW KATSKGWCWK |
| ef173422-03 | IVCYAEWPEY LSDNDASDVN KTSKPDISVC RFYTLDSKTW KATSKGWCWK |
| dq473490-04 | IVAYGEWPSF LDDSDASDVN KTTKPDISAC RFYTLDSKTW TNLSKGWCWK |
| hrv-05 | IVAYGEWPSF LSDVDASDVN KTTKPDTSAC RFYTLDSKMW TQGSKGWCWK |
| dq473486-06 | VVCYAEWPEY LPDGDASDVN KTSKPDTSVC RFYTLESKTW QNSSKGWCWK |
| l05355-14 | VVCYAEWPEY LPDVDASDVN KTSKPDTSVC RFYTLDSKTW TTGSKGWCWK |
| k02121-14 | VVCYAEWPEY LPDVDASDVN KTSKPDTSVC RFYTLDSKTW TTGSKGWCWK |
| ef173420-17 | VVAYAEWPSY LADIDASDVN KTSQPDTSVC RFYTLESKTW TRDSKGWCWK |
| hrv-26 | VVAYGEWPSF LSDVDASDVN KTTKPDTSAC RFYTLQSKMW NPDSKGWCWK |
| hrv-27 | IVAYAEWPSF LKDDDASDVN KPTRPDTSAC RFYTLASKTW SRESKGWCWK |
| ef173421-27 | IVAYAEWPSF LKDDDASDVN KPTRPDTSAC RFYTLASKTW SRESKGWCWK |
| dq473487-35 | VVCYAEWPEY LSDDDASDVN KTSKPDTSVC RFYTLDSKTW QQDSKGWCWK |
| hrv-35 | VVCYAEWPEY LSDDDASDVN KTSKPDTSVC RFYTLDSKTW QQDSKGWCWK |
| ef173423-37 | VVCYAEWPEY LPDKDASDVN KTSKPDTSVC RFYTLESKTW KNSSKGWCWK |
| hrv-42 | IVAYGEWPSF LSDVDASDVN KTTKPDTSAC RFYTLDSKTW SQSSKGWCWK |
| dq473488-48 | VVAYAEWPSY LSDMDASDVN KTSQSDTSVC RFYTLESKLW KTTSKGWCWK |
| hrv-52 | VVAYAEWPSY LSDSDASDVN KTSQPDTSVC RFYTLDSKTW SANSKGWCWK |
| hrv-52-f10 | VVAYAEWPSY LSDSDASDVN KTSQPDTSVC RFYTLDSKTW SANSKGWCWK |
| ef173424-52 | VVAYAEWPSY LSDSDASDVN KTSQPDTSVC RFYTLDSKTW SANSKGWCWK |
| hrv-69 | VVAYAEWPSY LTDTDASDVN KTSQPDTSVC RFYTLESKTW TNLSKGWCWK |
| dq473489-70 | VVAYAEWPSY LADNDASDVN KTSQPDTSVC RFYTLESKTW TNSSKGWCWK |
| hrv-72 | VVCYAEWPEY LPDRDASDVN KTSKPDTSVC RFYTLDSKMW QKNSKGWCWK |
| hrv-79 | VVCYAEWPEY LPDSDASDVN KTSKPDTSVC RFFTLDSKEW TQNSKGWCWK |
| hrv-83 | VVCYAEWPEY LPDKDASDVN KTSKPDTSVC RFYTLDSKDW SANSKGWCWK |
| hrv-84 | IVGYGEWPSF LDDKDASDVN KPTRPDTSAC RFYTLASKTW SQSSKGWCWK |
| hrv-86 | VVCYAEWPEY LPDKDASDVN KTSKPDTSVC RFYTLDSKTW NNTSKGWCWK |
| hrv-91 | VVAYAEWPSY LADSDASDVN KTSKPDTSVC RFYTLDSKTW TRESKGWCWK |
| hrv-92 | VVCYAEWPEY LSDNDASDVN KTSKPDTSVC RFYTLDSKDW SPSSKGWCWK |
| ef173425-93 | IVAYAEWPSF LEDTDASDVN KPTRPDTSAC RFYTLASKTW SPASRGWCWK |
| hrv-97 | VVAYAEWPSF LKDVDASDVN KPTRPDTSAC RFYTLASKTW NPTSKGWCWK |
| hrv-99 | VVAYGEWPSF LSDNDASDVN KTTKPDTSAC RFYTLDSKLW TQDSKGWCWK |
| | 151 200 |
| annotation | BHHHHHHHHA HHHHHHHHBB BBBDBBBBBB .......BBB EBBBBB.... |
| af499637-cva13 | LPDALKDMGL FGQNMYYHYL GRSGYTVHVQ CNASKFHQGT LGVFAVPEYC |
| af546702-cva21 | LPDCLKDMGM FGQNMYYHYL GRSGYTIHVQ CNASKFHQGA LGVFLIPEFV |
| v01149-pv1m | LPDALRDMGL FGQNMYYHYL GRSGYTVHVQ CNASKFHQGA LGVFAVPEMC |
| d00239-01 | LPDALKEMGI FGENMYYHFL GRSGYTVHVQ CNASKFHQGT LLVAMIPEHQ |
| pico-01 | LPDALKDMGI FGENMYYHFL GRSGYTVHVQ CNASKFHQGT LLVAMIPEHQ |
| hrv-01 | LPDALKDMGI FGENMYYHFL GRSGYTVHVQ CNASKFHQGT LLVAMIPEHQ |
| x02316-02 | LPDALKDMGI FGENMFYHYL GRSGYTIHVQ CNASKFHQGT LIVALIPEHQ |
| hrv-07 | LPDALKNMGI FGENMFYHFL GRSGYTIHVQ CNSSKFHQGL LIVAAIPEHQ |
| dq473503-07 | LPDALKNMGI FGENMFYHFL GRSGYTIHVQ CNSSKFHQGL LIVAAIPEHQ |
| hrv-08 | LPDALKDMGV FGENMFYHSL GRAGYMIHVQ CNASKFHSGT LLVVAIPEHQ |
| pico-09 | LPDALKDMGI FGQNMYYHFL GRSGYTVHVQ CNSSKFHQGT LIVAMIPEDQ |
| hrv-09 | LPDALKDMGI FGQNMYYHFL GRSGYTVHVQ CNSSKFHQGT LIVAMIPEHQ |
| hrv-09-f01 | LPDALKDMGI FGQNMYYHFL GRSGYTVHVQ CNSSKFHQGT LIVAMIPEHQ |
| hrv-09-f02 | LPDALKDMGI FGQNMYYHFL GRSGYTVHVQ CNSSKFHQGT LIVAMIPEHQ |
| dq473498-10 | LPDALKDMGI FGENMFYHYL GRSGYTVHVQ CNASKFHQGT LLVVMIPEHQ |
| hrv-10 | LPDALKDMGI FGENMFYHYL GRSGYTVHVQ CNASKFHQGT LLVVMIPEHQ |
| ef173414-11 | LPDALRDMGI FGENMFYHFL GRSGYTVHVQ CNASKFHQGT LIVAMIPEHQ |
| ef173415-12 | LPDALKDMGI FGENMFYHFL GRSGYTIHVQ CNASKFHQGT LMVVAIPEHQ |
| hrv-13 | LPDALKDMGI FGENMFYHFL GRCGYTVHVQ CNASKFHQGT LLIAMIPEHQ |
| hrv-13-f03 | LPDALKDMGI FGENMFYHFL GRSGYTVHVQ CNASKFHQGT LLIAMIPEHQ |
| dq473493-15 | LPDALRDMGV FGQNMYYHFL GRSGYTVHVQ CNSSKFHQGT LIVAMIPEHQ |
| l24917-16 | LPDALKDMGI FGENMFYHFL GRSGYTVHVQ CNASKFHQGT LLVVMIPEHQ |
| hrv-18 | LPDALKDMGI FGENMYYHFL GRSGYTVHVQ CNASKFHQGT LIVAMIPEHQ |
| hrv-19 | LPDALKDMGV FGQNMYYHFL GRSGYTIHVQ CNASKFHQGT LIVAAIPEHQ |
| hrv-20 | LPDALKNMGI FGENLFYHFL GRAGYTVHVQ CNASKFHQGT LIVAAIPEHQ |
| hrv-21 | LPDALKNMGI FGENMFYHFL GRSGYTVHVQ CNASKFHQGT LIVVMIPEHQ |
| hrv-22 | LPDALKDMGI FGQNMYYHFL GRSGYTIHVQ CNASKFHQGT LIVAAIPEHQ |
| dq473497-23 | LPDALRDMGI FGENMFYHYL GRSGYTVHVQ CNATKFHQGT LVVAMIPEHQ |
| hrv-24 | LPDALKEMGI FGENMFYHFL GRSGYTVHVQ CNASKFHQGT LIVAMIPEHQ |
| ef173416-24 | LPDALKEMGI FGENMFYHFL GRSGYTVHVQ CNASKFHQGT LIVAMIPEHQ |
| hrv-25 | LPDALKDMGI FGENMYYHYL GRSGYTVHVQ CNASKFHQGT LLVVMIPEHQ |
| dq473508-28 | LPDALKNMGI FGENLFYHFL GRAGYTVHVQ CNASKFHQGT LIVAAIPEHQ |
| hrv-29 | LPDALKDMGI FGENMYYHYL GRSGYTVHVQ CNASKFHQGT LLVVMIPEHQ |
| dq473512-30 | LPDALRDMGI FGENMFYHYL GRSGYTVHVQ CNATKFHQGT LIVAMIPEHQ |
| hrv-30 | LPDALRDMGI FGENMFYHYL GRSGYTVHVQ CNATKFHQGT LIVAMIPEHQ |
| hrv-31 | LPDALKDMGI FGENMYYHYL GRSGYTVHVQ CNASKFHQGT LLIMMIPEHQ |
| hrv-32 | LPDALKDMGV FGQNMYYHFL GRSGYTVHVQ CNSSKFHQGT LIVAMIPEHQ |
| hrv-33 | LPDALKDMGI FGENMFYHFL GRSGYTVHVQ CNASKFHQGT LIVAMIPEHQ |
| hrv-34 | LPDALKDMGI FGENMYYHFL GRSGYTVHVQ CNASKFHQGT LIVAMIPEHQ |
| dq473501-34 | LPDALKDMGI FGENMYYHFL GRSGYTVHVQ CNASKFHQGT LIVAMIPEHQ |
| dq473505-36 | LPDALKNMGI FGENMFYHFL GRSGYTIHVQ CNSSKFHQGL LIVAAIPEHQ |
| hrv-38 | LPDALKEMGV FGQNMYYHFL GRSGYTVHVQ CNSSKFHQGT LIVAMIPEHQ |
| dq473495-38 | LPDALKEMGV FGQNMYYHFL GRSGYTVHVQ CNSSKFHQGT LIVAMIPEHQ |
| ay751783-39 | LPDALKDMGI FGENMYYHFL GRSGYTVHVQ CNASKFHQGT LLIAMVPEHQ |
| hrv-40 | LPDALKDMGI FGENMFYHFL GRSGYTVHVQ CNASKFHQGT LLIVMIPEHQ |
| dq473491-41 | LPDALKDMGI FGENMFYHFL GRSGYTVHVQ CNASKFHQGT LLIATIPEHQ |
| hrv-43 | LPDALKDMGI FGQNMYYHFL GRSGYTVHVQ CNASKFHQGT LIVAMIPEHQ |
| dq473499-44 | LPDALKDMGI FGENMYYHYL GRSGYTVHVQ CNASKFHQGT LLVVMILEHQ |
| hrv-45 | LPDALKDMGV FGENMFYHAL GRSGYLIHVQ CNASKFHSGT LLVVAIPEHQ |
| dq473506-46 | LPDALKSMGI FGENMFYHHL GRAGYTIHVQ CNASKFHQGT LIVVAIPEHQ |
| hrv-47 | LPDALKDMGI FGENMYYHYL GRSGYTVHVQ CNASKFHQGT LLIMMIPEHQ |
| dq473496-49 | LPDALREMGI FGENMFYHYL GRSGYTIHVQ CNASKFHQGT LIVALIPEHQ |
| hrv-49-f04 | LPDALKEMGI FGENMFYHYL GRSGYTIHVQ CNASKFHQGT LIVALIPEHQ |
| hrv-50 | LPDALKEMGI FGENMYYHFL GRSGYTVHVQ CNASKFHQGT LIVAMIPEHQ |
| hrv-51 | LPDALKDMGI FGENMFYHFL GRTGYTIHVQ CNASKFHQGT LVVAAIPEHQ |
| dq473507-53 | LPDALKNMGI FGENLFYHFL GRAGYTVHVQ CNASKFHQGT LIVAAIPEHQ |
| hrv-54 | LPDALKDMGI FGENMFYHFL GRSGYTVHVQ CNASKFHQGT LLVAMVPEHQ |
| hrv-54-f05 | LPDALKDMGI FGENMFYHFL GRSGYTVHVQ CNASKFHQGT LLIAMVPEHQ |
| dq473511-55 | LPDALKNMGI FGENMFYHFL GRSGYTVHVQ CNASKFHQGT LIVAMIPEHQ |
| hrv-56 | LPDALKDMGI FGENMFYHFL GRSGYTVHVQ CNASKFHQGT LLVAMVPEHQ |
| hrv-57 | LPDALKNMGI FGENMFYHFL GRSGYTVHVQ CNASKFHQGT LVVAMIPEHQ |
| hrv-58 | LPDALKGMGI FGENMFYHFL GRSGYTIHVQ CNSSKFHQGL LIVAAIPEHQ |
| dq473500-59 | LPDALKDMGI FGENMFYHFL GRSGYTVHVQ CNASKFHQGT LLVAMVPEHQ |
| hrv-60 | LPDALKDMGV FGQNMYYHFL GRSGYTVHVQ CNSSKFHQGT LIVAMIPEHQ |
| hrv-61 | LPDALKDMGI FGENMFYHFL GRSGYTVHVQ CNASKFHQGT LLVAMIPEHQ |
| hrv-62 | LPDALKDMGI FGENMYYHYL GRSGYTVHVQ CNASKFHQGT LLVVMIPEHQ |
| hrv-63 | LPDALKDMGV FGENMFYHFL GRSGYTVHVQ CNASKFHQGT LLVAMVPEHQ |
| hrv-64 | LPDALKDMGI FGQNMYYHFL GRAGYTIHVQ CNASKFHQGT LIVAAIPEHQ |
| ef173417-64 | LPDALKDMGI FGQNMYYHFL GRAGYTIHVQ CNASKFHQGT LIVAAIPEHQ |
| hrv-65 | LPDALKNMGI FGENMFYHFL GRAGYTVHVQ CNASKFHQGT LVIAAIPEHQ |
| hrv-66 | LPDALKDMGI FGENMFYHYL GRSGYTVHVQ CNASKFHQGT LLVVMIPEHQ |
| hrv-67 | LPDALKDMGV FGQNMYYHFL GRSGYTVHVQ CNSSKFHQGT LIVAMIPEHQ |
| hrv-68 | LPDALKNMGI FGENLFYHFL GRAGYTVHVQ CNASKFHQGT LVVAAIPEHQ |
| hrv-71 | LPDALKNMGI FGENMFYHFL GRAGYTVHVQ CNASKFHQGT LVVAAIPEHQ |
| dq473492-73 | LPDALKDMGI FGENMFYHFL GRSGYTVHVQ CNASKFHQGT LLVAMIPEHQ |
| dq473494-74 | LPDALRDMGV FGQNMYYHFL GRNGYTVHVQ CNSSKFHQGT LIVAMIPEHQ |
| dq473510-75 | LPDALKDMGI FGQNMYYHFL GRSGYTVHVQ CNASKFHQGT LIVAMIPEHQ |
| dq473502-76 | LPDALKDMGI FGENMFYHFL GRSGYTVHVQ CNASKFHQGT LIVAMIPEHQ |
| hrv-76 | LPDALKDMGI FGENMFYHFL GRSGYTVHVQ CNASKFHQGT LIVAMIPEHQ |
| hrv-77 | LPDALKDMGI FGENMYYHYL GRSGYTVHVQ CNASKFHQGT LLVVMIPEHQ |
| hrv-78 | LPDALKDMGI FGENMFYHFL GRSGYTVHVQ CNASKFHQGT LLVVAIPEHQ |
| ef173418-78 | LPDALKDMGI FGENMFYHFL GRSGYTVHVQ CNASKFHQGT LLVVAIPEHQ |
| hrv-80 | LPDALKSMGI FGENMFYHHL GRAGYTIHVQ CNASKFHQGT LIVVAIPEHQ |
| hrv-81 | LPDALKDMGI FGENMFYHFL GRSGYTVHVQ CNASKFHQGT LLVTMIPEHQ |
| hrv-81-f06 | LPDALKDMGI FGENMFYHFL GRSGYTVHVQ CNASKFHQGT LLVTMIPEHQ |
| hrv-81-f07 | LPDALKDMGI FGENMFYHFL GRSGYTVHVQ CNASKFHQGT LLVTMIPEHQ |
| hrv-82 | LPDALKDMGV FGQNMYYHFL GRSGYTIHVQ CNASKFHQGT LIVAAIPEHQ |
| dq473509-82 | LPDALKEMGV FGQNMYYHFL GRSGYTIHVQ CNASKFHQGT LIVAAIPEHQ |
| hrv-85 | LPDALKDMGI FGENMYYHFL GRSGYTVHVQ CNASRFHQGT LLVVMIPEHQ |
| pico-85 | LPDALKDMGI FGENMYYHFL GRSGYTVHVQ CNASRFHQGT LLVVMIPEHQ |
| dq473504-88 | LPDALKSMGI FGENMFYHFL GRSGYTIHVQ CNSSKFHQGL LIVAAIPEHQ |
| a10937-89 | LPDALKNMGI FGENMFYHFL GRSGYTIHVQ CNSSKFHQGL LIVAAIPEHQ |
| hrv-89 | LPDALKNMGI FGENMFYHFL GRSGYTIHVQ CNSSKFHQGL LIVAAIPEHQ |
| hrv-89-f08 | LPDALKNMGI FGENMFYHFL GRSGYTIHVQ CNSSKFHQGL LIVAAIPEHQ |
| hrv-89-f09 | LPDALKNMGI FGENMFYHFL GRSGYTIHVQ CNSSKFHQGL LIVAAIPEHQ |
| m16248-89 | LPDALKNMGI FGENMFYHFL GRSGYTIHVQ CNSSKFHQGL LIVAAIPEHQ |
| hrv-90 | LPDALKGMGI FGENMFYHFL GRSGYTVHVQ CNASKFHQGT LIVAMIPEHQ |
| hrv-94 | LPDALKDMGI FGQNMYYHFL GRSGYTIHVQ CNASKFHQGT LIVAAIPEHQ |
| ef173419-94 | LPDALKDMGI FGQNMYYHFL GRSGYTIHVQ CNASKFHQGT LIVAAIPEHQ |
| hrv-95 | LPDALKDMGV FGENMFYHSL GRAGYLIHVQ CNASKFHSGT LLVVAIPEHQ |
| hrv-96 | LPDALKDMGI FGENMFYHFL GRSGYTVHVQ CNASKFHQGT LLVVMIPEHQ |
| hrv-98 | LPDALKDMGI FGENMFYHFL GRSGYTVHVQ CNASKFHQGT LLVAMVPEHQ |
| hrv-100 | LPDALKDMGI FGENMFYHYL GRSGYTVHVQ CNASKFHQGT LLVVMIPEHQ |
| ef186077-qpm | LPDCLRDMGV FGQNMYHHAM GRSGYIIHTQ CNATKFHSGC LLVAVVPEHQ |
| ef077279-nat001 | LPDCLKDMGI FGQNMFHHAM GRSGYIIHTQ CNATKFHSGC LLVAVVPEHQ |
| ny1078 | LPDCLKNMGI FGQNMYYHAM GRSGYIIHTQ CNATKFHSGC LLVAVVPEHQ |
| ef582385-c024 | LPDCLKDMGI FGQNLYHHAL GRSGYIIHTQ CNATKFNSGC LIVAVVPEHQ |
| ef077280-nat045 | IPDCLKDMGM FGQNMYHHSM GRSGMLIHVQ CNATKFHSGC LLVVVVPEHQ |
| ef582387-c026 | LPDCLRDMGV FGQNMYHHAM GRSGYIIHTQ CNATKFHSGC LLVAVVPEHQ |
| ef582386-c025 | IPDCLKDMGM FGQNMYYHAL GRSGYIFHVQ CNATKFHSGL LLVAIVPEHQ |
| dq473485-03 | LPDALKDMGV FGQNMFYHSL GRTGYTIHVQ CNATKFHSGC LLVVVIPEHQ |
| ef173422-03 | LPDALKDMGV FGQNMFYHSL GRTGYTIHVQ CNATKFHSGC LLVVVIPEHQ |
| dq473490-04 | LPDALKDMGI FGQNMFFHSQ GRTGYTIHVQ CNATKFHSGC LLVVVIPEHQ |
| hrv-05 | LPDALKDMGI FGQNMFFHSQ GRTGYTIHVQ CNATKFHSGC LLVVVIPEHQ |
| dq473486-06 | LPDALKDMGV FGQNMFFHSL GRSGYTIHVQ CNATKFHSGC LLVVVIPEHQ |
| 105355-14 | LPDALKDMGV FGQNMFFHSL GRSGYTVHVQ CNATKFHSGC LLVVVIPEHQ |
| k02121-14 | LPDALKDMGV FGQNMFFHSL GRSGYTVHVQ CNATKFHSGC LLVVVIPEHQ |
| ef173420-17 | LPDALKDMGV FGQNMFFHAL GRTGYTIHVQ CNATKFHSGC LLVVVVPEHQ |
| hrv-26 | LPDALKEMGI FGQNMFFHAQ GRTGYTIHVQ CNATKFHSGC LLVVVIPEHQ |
| hrv-27 | LPDALKDMGI FGQNMFYHSQ GRTGYTIHVQ CNATKFHSGT LLVVIIPEHQ |
| ef173421-27 | LPDALKDMGI FGQNMFYHSQ GRTGYTIHVQ CNATKFHSGT LLVVIIPEHQ |
| dq473487-35 | LPDALKDMGV FGQNMFYHTL GRTGYTIHVQ CNATKFHSGC LLIVVIPEHQ |
| hrv-35 | LPDALKDMGV FGQNMFYHTL GRTGYTIHVQ CNATKFHSGC LLXVVIPEHQ |
| ef173423-37 | LPDALKDMGV FGQNMFFHSL GRSGYTIHVQ CNATKFHSGC LLVVVIPEHQ |
| hrv-42 | LPDALKDMGI FGQNMFFHSQ GRTGYTIHVQ CNATKFHSGC LLVVVIPEHQ |
| dq473488-48 | LPDALKDMGV FGQNMFFHSL GRTGYTIHVQ CNATKFHSGC LLVVVVPEHQ |
| hrv-52 | LPDALKDMGV FGQNMFFHAL GRTGYTVHVQ CNATKFHSGC LLVVAIPEHQ |
| hrv-52-f10 | LPDALKDMGV FGQNMFFHAL GRTGYTVHVQ CNATKFHSGC LLVVAIPEHQ |
| ef173424-52 | LPDALKDMGV FGQNMFFHAL GRTGYTVHVQ CNATKFHSGC LLVVAIPEHQ |
| hrv-69 | LPDALKEMGV FGQNMFFHAL GRTGYTIHVQ CNATKFHSGC LLVVVIPEHQ |
| dq473489-70 | LPDALKDMGV FGQNMFFHAL GRTGYTIHVQ CNATKFHSGC LLVVVVPEHQ |
| hrv-72 | LPDALKDMGV FGQNMFFHSL GRSGYTIHVQ CNATKFHSGC LLVVVIPEHQ |
| hrv-79 | LPDALKDMGV FGQNMFFHSL GRTGYTIHVQ CNATKFHSGC LLVVVIPEHQ |
| hrv-83 | LPDALKDMGV FGQNMFFHSL GRTGYTIHVQ CNATKFHSGC LLVVVIPEHQ |
| hrv-84 | LPDALKDMGI FGQNMFYHSQ GRTGYTIHVQ CNATKFHSGT LLVVVIPEHQ |
| hrv-86 | LPDALKDMGV FGQNMFFHSL GRSGYTIHVQ CNATKFHSGC LLVVVIPEHQ |
| hrv-91 | LPDALKDMGV FGQNMFFHAL GRTGYTIHVQ CNATKFHSGC LLVVVVPEHQ |
| hrv-92 | LPDALKDMGV FGQNMFFHSL GRTGYTIHVQ CNATKFHSGC LLVVVIPEHQ |
| ef173425-93 | LPDALKDMGI FGQNMFFHSQ GRTGYTIHVQ CNATKFHSGT LLVVIIPEHQ |
| hrv-97 | LPDALKDMGI FGQNMFYHSQ GRTGYTIHVQ CNATKFHSGT LLVVIIPEHQ |
| hrv-99 | LPDALKDMGI FGQNMFYHSQ GRTGYTIHVQ CNATKFHSGC LLVVVIPEHQ |
| | 201 250 |
| annotation | .......... ..PPPPPPPP PPPPPPPPPP PP........ .....HHHHH |
| af499637-cva13 | LAGDSNSKTN YTSYVNANPG EAGGKFVSTF TPD.TGTSPK REFQPVDYLF |
| af546702-cva21 | MACNTESKTS YVSYINANPG ERGGEFTNTY NPSNTDASEG RKFAALDYLL |
| v01149-pv1m | LAGDSNTTTM HTSYQNANPG EKGGTFTGTF TPDNNQTSPA RRFCPVDYLL |
| d00239-01 | LASAKNGSVT .AGYNLTHPG EAGRVVGQQ. .RDANLRQPS D....DSWLN |
| pico-01 | LASAKHGSVT .AGYKLTHPG EAGRDVSQE. .RDASLRQPS D....DSWLN |
| hrv-01 | LASAKHGSVT .AGYKLTHPG EAGRDVSQE. .RDASLRQPS D....DSWLN |
| x02316-02 | IASALHGNVN .VGYNYTHPG ETGREVKAET R.LNPDLQPT E....EYWLN |
| hrv-07 | LASAVKGNVS .VGYKHTHPG EQGREISSE. RRTDDDRRPS D....DSWLN |
| dq473503-07 | LASAVKGNVS .VGYKHTHPG EQGREISSE. RRTDDDRRPS D....DSWLN |
| hrv-08 | LSYLGTGNVT .VGYRHTHPG ENGREIAEVA NR..NERQPS Y....DSWLN |
| pico-09 | LAAAERGNVT .AGYNFTHPG EPGREVGKPN R.QYTSKQPS D....DNWLN |
| hrv-09 | LAAAERGNVT .AGYNFTHPG EPGREVGKPN R.QYTSKQPS D....DNWLN |
| hrv-09-f01 | LAAAERGNVT .AGYNFTHPG EPGREVGKPN R.QYTSKQPS D....DNWLN |
| hrv-09-f02 | LAAAERGNVT .AGYNFTHPG EPGREVGKPN R.QYTSKQPS D....DNWLN |
| dq473498-10 | LASAHTGNVT .AGYNFTHPG EKGRTVGD.. RRDHHDKQPS D....DNWLN |
| hrv-10 | LASAHTGNVT .AGYNFTHPG EKGRTVGD.. RRDHHDKQPS D....DNWLN |
| ef173414-11 | LASATTGNVT .AGYKLTHPG ERGRDVSLTD R.QDITKQPS D....DSWLN |
| ef173415-12 | LAYIGTGNVT .VGYKHTHPG ESGRIIANNA ..DNNTRQPS D....DNWLN |
| hrv-13 | LATAAGGSVS .AGYKYTHPG EKGRDVGKIN R.ERNDRQPS D....DNWLN |
| hrv-13-f03 | LATAAGGSVS .AGYKYTHPG EKGRDVGKTG R.ERNDRQPS D....DNWLN |
| dq473493-15 | LASAEKGNVT .AGYKLTHPG EAGRDVSITN R.DHVSRQPS D....ENWLN |
| l24917-16 | LATVNKGNVN .AGYKYTHPG EAGREVGTQ. ..VENEKQPS D....DNWLN |
| hrv-18 | LASAIGGNVT .AGYKYTHPG ERGRVINVAS R.GDNEKQPS D....DNWLN |
| hrv-19 | LASATKGNVT .AGYNYTHPG EMGRDVGKIG R.ENNGKQPS D....DNWLN |
| hrv-20 | LAYIDKGSVT .VGYNHTHPG ETGRVIGGFT ..NNNSRQPS D....ENWLN |
| hrv-21 | LASASTGNVT .AGYNLTHPG EQGRDVGI.T RVEDLLKQPS D....DSWLN |
| hrv-22 | LASAGRGNVA .AGYNYTHPG EIGRDVGKEN R.EQANNQPS D....DNWLN |
| dq473497-23 | IASLQHGDVN .VGYNFTHPG EEGREVKNTT R.EHLQQQPT E....EHWLN |
| hrv-24 | LASATTGNVR .AGYNLTHPG ESGRNVDPIT R.DNPTKQPS D....DSWLN |
| ef173416-24 | LASATTGNVR .AGYNLTHPG ESGRNVDPIT R.DNPTKQPS D....DSWLN |
| hrv-25 | LANADSTKVG .AGYNYTHPG ERGRQVGD.A RDPITAVHPS D....DNWLN |
| dq473508-28 | LAFISKGNVT .VGYNYTHPG EQGREIGTFG PSSTLDRQPS D....ENWLN |
| hrv-29 | LASVGTDKVG .PGYAFTHPG EAGRQIGNVS ..EPTSKHPS D....DNWLN |
| dq473512-30 | IASLQHGNVN .VGYKFTHPG EEGREVRSN. RTEYLGQQPT E....EYWLN |
| hrv-30 | IASLQHGNVN .VGYKFTHPG EEGREVRSN. RTEYLGQQPT E....EYWLN |
| hrv-31 | LANENRTRAD .AGYNFTHPG ELGREVGN.. RRNPHDNLPS D....DNWLN |
| hrv-32 | LAAAERGNVT .AGYNFTHPG EIGRDVGKTE R.QHTSKQPS D....DNWLN |
| hrv-33 | LASATTGNAT .AGYNLTHPG EAGRDVSPKT R.ENIDKQPS D....DAWLN |
| hrv-34 | LASATYGNVA .AGYNYTHPG ERGRVINVTT R.EQNDKQPS D....DNWLN |
| dq473501-34 | LASATYGNVA .AGYNYTHPG ERGRVINVTT R.EQNDKQPS D....DNWLN |
| dq473505-36 | LASATSGNVS .VGYNHTHPG EQGREVVPS. RNSEDNKRPS D....DSWLN |
| hrv-38 | LAAAEKGNVT .AGYNLTHPG ETGRDVGITV R.EHPNKQPS D....DNWLN |
| dq473495-38 | LAAAEKGNVT .AGYNLTHPG ETGRDVGITV R.EHPNKQPS D....DNWLN |
| ay751783-39 | LASANYGNVT .AGYNYTHPG EAGRDVGQQ. .RTNNEKQPS D....DNWLN |
| hrv-40 | LASATTGNVT .AGYNYTHPG ELGRTVGQR. RSEVNIKQPS D....DNWLN |
| dq473491-41 | LATAVGGSVS .AGYNYTHPG EKGRDVGKM. DRERNDRQPS D....DSWLN |
| hrv-43 | LASATHGNVT .AGYNFTNPG EAGRNVGQI. RNEXLTKQPS D....DNWLN |
| dq473499-44 | LASVGTEKVG .PGYNFTHPG EAGRQIGNVS DR..TSKHPS D....DNWLN |
| hrv-45 | LAYIGTGNVT .VGYKHTHPG ETGRVIATTT DK..QTRQPS F....DSWLN |
| dq473506-46 | LAYIGGGNVT .VGYNHTHPG ESGRRIGGF. DGTERNRQPS D....ENWLN |
| hrv-47 | LANENSTRAD .AGYNYTHPG ERGREVGGE. RISHANL.PS D....DNWLN |
| dq473496-49 | IASALHGDVN .VGYNFTHPG ETGREVKSD. RSMNLNLQPT E....EYWLN |
| hrv-49-f04 | IASALHGDVN .VGYKYTHPG EAGREVKSD. RNINLNVQPT E....EYWLN |
| hrv-50 | LASATHGNVT .AGYNYTHPG ERGRTINVVN R.DQNDKQPS D....DNWLN |
| hrv-51 | LAFIGKGNVS .VGYRHTHPG ENGRTLNGS. RVDGNSKQPT E....ESWLN |
| dq473507-53 | LAFIKSGNVT .VGYKYTHPG EQGREIGTFG ENSVLDRQPS D....ENWLN |
| hrv-54 | LASASRGNVT .AGYNYTHPG ELGRVVGQ.A RVQTNDNQPS D....DNWLN |
| hrv-54-f05 | LASASRGNVT .AGYNYTHPG ELGRVVGQP. RTQINDHQPS D....DNWLN |
| dq473511-55 | LASATSGNVT .AGYNLTHPG EQGRDVSVTG R.QNATKQPS D....DNWLN |
| hrv-56 | LASANKGNVT .AGYNYTHPG EAGRDVGQ.T RV.NNDRQPS D....DNWLN |
| hrv-57 | LASATYGNVA .AGYRLTHPG EAGRDVSL.S RSENTEKQPS D....DNWLN |
| hrv-58 | LASATSGNVW .VGYNHTHPG EQGREVV.PT RSSTDNKRPS D....DSWLN |
| dq473500-59 | LASANIGNVT .VGYNYTHPG ESGRDVGQD. .RRSNDKQPS D....DNWLN |
| hrv-60 | LAAAEKGNVT .AGYNLTHPG EKGRTIGIPD R.LHPSKQPS D....DNWLN |
| hrv-61 | LATAVGGSVS .AGYNYTHPG EKGRDVGKLN R.ESNPRQPS D....DNWLN |
| hrv-62 | LANANSAKAG .AGYNYTHPG ERGRQVGDP. RENITAAHPS D....DNWLN |
| hrv-63 | LASASAGNVT .AGYNYTHPG EAGRDVGQ.N R.DANYKQPS D....DNWLN |
| hrv-64 | LASAHKGNVT .TGYNYTHPG ESGRDVSVEV R.DTANYQPS D....DAWLN |
| ef173417-64 | LASAHKGNVT .TGYNYTHPG ESGRDVSVEV R.DTANYQPS D....DAWLN |
| hrv-65 | LAYIGSGNVS .VGYKQTHPG ENGRTIDD.T RVNTMSNQPT D....ENWLN |
| hrv-66 | LAAASTGNVA .AGYNFTHPG EKGREVGSP. .RETHPNHPS D....DSWLN |
| hrv-67 | LAAAERGNVT .AGYNFTHPG ETGRDVGKTE R.VYQNKQPS D....DNWLN |
| hrv-68 | LAYIGKGNVT .VGYNHTHPG EKGRRIGGF. .IDNESRQPS D....ENWLN |
| hrv-71 | LAYIGSGNVS .VGYRHTHPG EQGRVIGT.. RENRNTNQPT D....ENWLN |
| dq473492-73 | LAAAKGGTVT .AGYKYTHPG EAGRDVGKTD RK.QNNRQPS D....DNWLN |
| dq473494-74 | LASAERGNVT .AGYKLTHPG EAGRDVSITT R.EHTSRQPS D....DNWLN |
| dq473510-75 | LASARHGNLT .AGYNFTHPG ENGRNVGQL. RTEYLDKQPS D....DNWLN |
| dq473502-76 | LASATTGNAT .AGYNLTHPG EGGRDVSLSN R.ADLSKQPS D....DAWLN |
| hrv-76 | LASATTGNAT .AGYNLTHPG EGGRDVSLSN R.ADLSKQPS D....DAWLN |
| hrv-77 | LASASSGNVT .AGYNFTHPG EQGREVG.ST R.ESHLKHPS D....DNWLN |
| hrv-78 | LAYIGTGNVT .VGYKHTHPG ESGRIIATNS .VNNNTRQPS D....DNWLN |
| ef173418-78 | LAYIGTGNVT .VGYKHTHPG ESGRIIATNS .VNNNTRQPS D....DNWLN |
| hrv-80 | LAYVGGGNVT .VGYNHTHPG ETGRRIGGYE NV.PRNRQPS D....ENWLN |
| hrv-81 | LATVNKANVT .AGYKYTHPG ESGRVVGTQA ...DDTKQPS D....DNWLN |
| hrv-81-f06 | LATVNKANVT .AGYKYTHPG ESGRVVGTQA ...DDTRQPS D....DNWLN |
| hrv-81-f07 | LATVNKANVT .AGYKYTHPG ESGRVVGTQA ...DDTRQPS D....DNWLN |
| hrv-82 | LASAERGNVN .TGYNYTHPG ESGRDVG.KD R.ESTRYQPS D....DNWLN |
| dq473509-82 | LASAEKGNVN .TGYNYTHPG ERGRDVGQ.E R.ESTSYQPS D....DNWLN |
| hrv-85 | LASATKGNVT .AGYNYTHPG ELGRIVGQ.T RAEINNKQPS D....DNWLN |
| pico-85 | LASATKGNVT .AGYNYTHPG ELGRIVGQ.T RAEINNKQPS D....DNWLN |
| dq473504-88 | LASALTGNVS .VGYKHTHPG EQGREISS.N RSSEDNRRPS D....DSWLN |
| a10937-89 | LASATSGNVS .VGYNHTHPG EQGREVV.PS RTSSDNKRPS D....DSWLN |
| hrv-89 | LASATSGNVS .VGYNHTHPG EQGREVV.PS RTSSDNKRPS D....DSWLN |
| hrv-89-f08 | LASATSGNVS .VGYNHTHPG EQGREVV.PS RTSSDNKRPS D....DSWLN |
| hrv-89-f09 | LASATSGNVS .VGYNHTHPG EQGREVV.PS RSSSDNKRPS D....DSWLN |
| m16248-89 | LASATSGNVS .VGYNHTHPG EQGREVV.PS RTSSDNKRPS D....DSWLN |
| hrv-90 | LASALAGNVT .AGYNLTHPG EGGRTVDQTN R.ENQSKQPS D....DSWLN |
| hrv-94 | LASAHKGNVN .TGYNYTHPG ETGRDVGTEV R.DTANYQPS D....DAWLN |
| ef173419-94 | LASAHKGNVN .TGYNYTHPG ETGRDVGTEV R.DTANYQPS D....DAWLN |
| hrv-95 | LSYLGTGNVT .VGYRHTHPG ENGREIAEVA NRN..ERQPS Y....DSWLN |
| hrv-96 | LATAAGGSVS .AGYNYTHPG EKGRDVGKEG R.EANVRQPS D....DNWLN |
| hrv-98 | LASASRGNVT .AGYNYTHPG ELGRVVGQ.A RTQTSGNQPS D....DNWLN |
| hrv-100 | LASAHTGNVT .AGYNFTHPG EKGRTVGDER KH..REKQPS D....DNWLN |
| ef186077-qpm | LAYIGGDNTR .VKYKHTHPG ELGHKIGSN. .SERGDNQPD E....NPFFN |
| ef077279-nat001 | LAYIGAD.AR .VSYEHTHPG EKGHVIGSN. .SDRNNHQPD E....SAFFN |
| ny1078 | LAYIGVD.AQ .VSYKHTHPG ERGHEIGRN. .TNRDDNQPD E....NPFFN |
| ef582385-c024 | LAYIGEANVN .VGYDHTHPG EGGHVIGSN. .VRRDNKQPD E....DPFFN |
| ef077280-nat045 | LAYIGAGGVN .VKYEHTHPG ERGHTLQAS. .DVRSNHNPD E....DPFYL |
| ef582387-c026 | LAYIGGTNAQ .VKYKHTHPG EQGHRIGSN. .SERNENQPD E....NPFFN |
| ef582386-c025 | LAYIGGTNAN .VGYNHTHPG EGGHEIGKN. .TERNDKQPD E....DPLFN |
| dq473485-03 | LASHEGGTVS .VKYKYTHPG DRGIDLDTVE VA....GGPT S....DAIYN |
| ef173422-03 | LASHEGGTVS .VKYKYTHPG DRGIDLDTVE VA....GGPT S....DAIYN |
| dq473490-04 | LASIQGGSVS .VYYEKTHPG EKGIDLNESD .....GDGPM L....DPLYM |
| hrv-05 | LASAEGGNVS .VLYDKTHPG EKGIDLSEAD .....STGPM K....DPLYM |
| dq473486-06 | LASHKGGNVS .VKYKYTHPG ESGIDLSTQL DE....DGPV K....DPVYS |
| 105355-14 | LASHEGGNVS .VKYTFTHPG ERGIDLSSAN EV....GGPV K....DVIYN |
| k02121-14 | LASHEGGNVS .VKYTFTHPG ERGIDLSSAN EV....GGPV K....DVIYN |
| ef173420-17 | LASHTEGNVS .VKYKYTHPG EQGIELDSAA EE....GGAS H....DPVYS |
| hrv-26 | LASIKGANVS .VLYDKTHPG ESGIDLNTTD ED.....GPM K....DPLYM |
| hrv-27 | LASAEGGNVS .VFYDQTHPG ERGIDLGTVI E.....KGPM T....DPIYN |
| ef173421-27 | LASAEGGNVS .VFYDQTHPG ERGIDLGTVI E.....KGPM T....DPIYN |
| dq473487-35 | LASHDGGTVS .VKYKFTHPG DQGIDLSTAE ET....NGPV K....NPVYN |
| hrv-35 | LASHDGGTVS .VKYKFTHPG DQGIDLSTAE ET....NGPV K....NPVYN |
| ef173423-37 | LASHEGSNVS .VKYKFTHPG ENGIDLELPP EK....DGPV K....NPVYG |
| hrv-42 | LASIEGGNVS .VLYDKTHPG EKGIDLND.E DP....KGPM K....DPLYM |
| dq473488-48 | LASRTEGNVS .VNYKYTHPG ERGIDLDEAE KE....GGAT H....NPVYS |
| hrv-52 | LASHTQGNVS .VKYKYTHPG EQGINLNDIP EE....GGAS H....DPVYS |
| hrv-52-f10 | LASHTQGNVS .VKYKYTHPG ESGINLDDTP EA....GGAS H....DPVYS |
| ef173424-52 | LASHTQGNVS .VKYKYTHPG EQGINLNDIP EE....GGAS H....DPVYS |
| hrv-69 | LASHTEGNVS .VKYKYTHPG ETGINLSDAV AT....GGAS H....DPVYS |
| dq473489-70 | LASHTQGNVS .VKYKYTHPG EQGIDLDSVA ET....GGAS H....DPVYS |
| hrv-72 | LASHEGGNVS .VKYKLTHPG EGGIDLSTNI EA....DGPV K....DPVYN |
| hrv-79 | LASHDGGTVS .VKYEFTHPG DQGIDLSTAE VQ....FGPA K....DAIYS |
| hrv-83 | LASHDGGTVS .VKYKFTHPG EQGIDLGTSG ER....TGPA K....DAVYN |
| hrv-84 | LASAKGGNVS .VYYDKTHPG ENGIDLSVA. DE....LGPK T....DPVYN |
| hrv-86 | LASHQGGNVS .VKYTLTHPG EAGIDLSKAE VD....KGPS N....NPVYS |
| hrv-91 | LASHTEGNVS .VKYKYTHPG EQGIELSNLA EE....GGAS H....DPVYS |
| hrv-92 | LASHDGGSVS .VKYKYTHPG DQGIDLGTTE EN....FGPA K....DAVYS |
| ef173425-93 | LASANGGNVS .VLYDKTHPG ENGIDLGS.E VK....DGPM T....DPIYN |
| hrv-97 | LASAEGGNVS .VLYDKTHPG ENGIDLNTN. DA....KGPK T....DPIYN |
| hrv-99 | LASIEGANVS .VLYDKTHPG EQGIDLSKT. DP....KGPV R....DPLYM |
| | 251 300 |
| annotation | HH..HHBHHH ..BBBFBBB. ..BBBBGBB. .......... ....BBBBBH |
| af499637-cva13 | GCGVMAGNAF VFPHQIINLR TNNCATLVLP YVNSLAIDCM AKHNNWGIVI |
| af546702-cva21 | GSGVLAGNAF VYPHQIINLR TNNSATIVVP YVNSLVIDCM AKHNNWGIVI |
| v01149-pv1m | GNGTLLGNAF VFPHQIINLR TNNCATLVLP YVNSLSIDSM VKHNNWGIAI |
| d00239-01 | FDGTLLGNLL IFPHQFINLR SNNSATLIVP YVNAVPMDSM LRHNNWSLVI |
| pico-01 | FDGTLLGNLL IFPHQFINLR SNNSATLIVP YVNAVPMDSM LRHNNWSLVI |
| hrv-01 | FDGTLLGNLL IFPHQFINLR SNNSATLIVP YVNAVPMDSM LRHNNWSLVI |
| x02316-02 | FDGTLLGNIT IFPHQFINLR SNNSATIIAP YVNAVPMDSM RSHNNWSLVI |
| hrv-07 | FDGTLLGNLP IYPHQYINLR TNNSATLILP YINAVPMDSM LRHNNWSLVI |
| dq473503-07 | FDGTLLGNLP IYPHQYINLR TNNSATLILP YINAVPMDSM LRHNNWSLVI |
| hrv-08 | CNGTLLGNAL IFPHQFINLR TNNAATLILP YVNATPMDFM LRHNNWSLLI |
| pico-09 | FDGTLLGNIT IYPHQFINLR SNNSATIIVP YVNAVPMDSM LRHNNWSLVI |
| hrv-09 | FDGTLLGNIT IYPHQFINLR SNNSATIIVP YVNAVPMDSM LRHNNWSLVI |
| hrv-09-f01 | FDGTLLGNIT IYPHQFINLR SNNSATIIVP YVNAVPMDSM LRHNNWSLVI |
| hrv-09-f02 | FDGTLLGNIT IYPHQFINLR SNNSATIIVP YVNAVPMDSM LRHNNWSLVI |
| dq473498-10 | FDGTSLGNAL IFPHQFINLR SNNSATIIVP YVNAVPMDSM LRHNNWSLVI |
| hrv-10 | FDGTSLGNAL IFPHQFINLR SNNSATIIVP YVNAVPMDSM LRHNNWSLVI |
| ef173414-11 | FDGTLLGNIT IYPHQFINLR SNNSATIIVP YVNAVPMDSM LRHNNWSLVI |
| ef173415-12 | CDGTLMGNLP IFPHQFINLR TNNSATLILP YMNCVSMDSM LRHNNWSLLI |
| hrv-13 | FDGTLLGNLT IFPHQFINLR SNNSATIIVP YVNAVPMDSM LRHNNWSLVI |
| hrv-13-f03 | FDGTLLGNLT IFPHQFINLR SNNSATIIVP YVNAVPMDSM LRHNNWSLVI |
| dq473493-15 | FDGTLLGNIT IFPHQFINLR SNNSATLIVP YVNAVPMDSM LRHNNWSLVI |
| l24917-16 | FDGTLLGNLL IFPHQFINLR SNNSATLIVP YVNAVPMDSM VRHNNWSLVI |
| hrv-18 | FDGTLLGNIT IYPHQLINLR SNNSATIIVP YVNAVPMDSM LRHNNWSLVI |
| hrv-19 | FDGTLLGNIT IFPHQFINIR SNNSATLILP YVNAVPMDSM LRHNNWSLVI |
| hrv-20 | CDGTLLGNIT IYPHQYINLR SNNSATLILP YVNAVPMDSM VRHNNWSIVI |
| hrv-21 | FDGTLLGNIT IFPHQFINLR SNNSATIIVP YVNAVPMDSM PRHNNWSLVI |
| hrv-22 | FDGTLLGNIT IFPHQFINLR SNNSATLILP YVNAVPMDSM LRHNNWSLVI |
| dq473497-23 | FDGTLLGNIT IFPHQFINLR SNNSATIIVP YVNAVPMDSM RSHNNWSLVI |
| hrv-24 | FDGTLLGNIT IFPHQFINLR SNNSATIIVP YVNAVPMDSM LRHNNWSLVI |
| ef173416-24 | FDGTLLGNIT IFPHQFINLR SNNSATIIVP YVNAVPMDSM LRHNNWSLVI |
| hrv-25 | FDGTMLGNVL IFPHQFINLR SNNSATIIVP YVNAVPMDSM LRHNNWSLVI |
| dq473508-28 | CDGTLLGNIT VYPHQFINLR SNNSATLILP YVNAVPMDSM VRHNNWSIVI |
| hrv-29 | FDGTLLGNVL IFPHQFINLR SNNSATIIVP YVNAVPMDSM LRHNNWSLVI |
| dq473512-30 | FDGTLLGNIT IFPHQFINLR SNNSATIIVP YVNAVPMDSM RSHNNWSLVI |
| hrv-30 | FDGTLLGNIT IFPHQFINLR SNNSATIIVP YVNAVPMDSM RSHNNWSLVI |
| hrv-31 | FDGTLLGNAL IYPHQFINLR SNNSATLIVP YVNAVPMDSM LRHNNWSLVI |
| hrv-32 | FDGTLLGNIT IFPHQFINLR SNNSATIIVP YVNAVPMDSM LRHNNWSLVI |
| hrv-33 | FDGTLLGNIT VYPHQLINLR SNNSATIIVP YVNAVPMDSM LRHNNWSLVI |
| hrv-34 | FDGTLLGNIT IYPHQLINLR SNNSATIIVP YVNAVPMDSM LRHNNWSLVI |
| dq473501-34 | FDGTLLGNIT IYPHQLINLR SNNSATIIVP YVNAVPMDSM LRHNNWSLVI |
| dq473505-36 | FDGTLLGNLP IYPHQYINLR TNNSATLILP YVNAVPMDSM LRHNNWSLVI |
| hrv-38 | FDGTLLGNIT IFPHQFINLR SNNSATIIVP YVNAVPMDSM LRHNNWSLVI |
| dq473495-38 | FDGTLLGNIT IFPHQFINLR SNNSATIIVP YVNAVPMDSM LRHNNWSLVI |
| ay751783-39 | FDGTLLGNLL IFPHQFINLR SNNSATIIVP YVNAVPMDSM LRHNNWSLLI |
| hrv-40 | FDGTFLGNLL IFPHQFINLR SNNSATIIVP YVNAVPMDSM LRHNNWSLLI |
| dq473491-41 | FDGTLLGNLT IFPHQFINLR SNNSATIIVP YVNAVPMDSM LRHNNWSLVI |
| hrv-43 | FDGTLLGNIT IFPHQFINLR SNNSATIIVP YVNAVPMDSM LRHNNWSLVI |
| dq473499-44 | FDGTLLGNVL IFPHQFINLR SNNSATIIVP YVNAVPMDSM LRHNNWSLVI |
| hrv-45 | CNGTLLGNAL IFPHQFINLR TNNAATLILP YVNATPMDSM LRHNNWSLLI |
| dq473506-46 | CNGTLLGNLT IFPHQFINLR SNNSATLILP YVNAVPMDSM LRHNNWSLVI |
| hrv-47 | FDGTLLGNAL IFPHQFINLR SNNSATLIVP YVNAVPMDSM LRHNNWSLVI |
| dq473496-49 | FDGTLLGNIT IFPHQFINLR SNNSATIIAP YVNAVPMDSM RSHNNWSLVI |
| hrv-49-f04 | FDGTLLGNIT IFPHQFINLR SNNSATIIAP YVNAVPMDSM RSHNNWSLVI |
| hrv-50 | FDGTLLGNIT IYPHQLINLR SNNSATIIVP YVNAVPMDSM PRHNNWSLVI |
| hrv-51 | CDGTLLGNIT IYPHQFINLR SNNSATLILP YVNAVPMDSM LRHNNWSLVI |
| dq473507-53 | CDGTLLGNIT VYPHQYINLR SNNSATLILP YVNAVPMDSM VRHNNWSIVI |
| hrv-54 | FDGTFLGNLL IFPHQFINLR TNNSATIIVP YVNAVPMDSM LRHNNWSLLI |
| hrv-54-f05 | FDGTFLGNLL IFPHQFINLR SNNSATIIVP YVNAVPMDSM LRHNNWSLLI |
| dq473511-55 | FDGTLLGNIT IYPHQLINLR SNNSATIIVP YVNAVPMDSM LRHNNWSLVI |
| hrv-56 | FDGTFMGNLL IFPHQFINLR SNNSATIIVP YVNAVPMDSM LRHNNWSLLI |
| hrv-57 | FDGTLLGNIT IFPHQFINLR SNNSATIIVP YVNAVPMDSM LRHNNWSLVI |
| hrv-58 | FDGTLLGNLP IYPHQYINLR TNNSATLILP YVNAVPMDSM LRHNNWSLVI |
| dq473500-59 | FDGTLLGNLL IFPHQFINLR SNNSATIIVP YVNAVPMDSM LRHNNWSLLI |
| hrv-60 | FDGTLLGNIT IFPHQFINLR SNNSATIIVP YVNAVPMDSM LSHNNWSLVI |
| hrv-61 | FDGTLLGNLT IFPHQFINLR SNNSATIIVP YVNAVPMDSM LRHNNWSLVI |
| hrv-62 | FDGTMLGNVL IFPHQFINLR SNNSATIIVP YVNAVPMDSM LRHNNWSLVI |
| hrv-63 | FDGTLLGNLL IFPHQFINLR SNNSATIIVP YVNAVPMDSM LRHNNWSLLI |
| hrv-64 | FDGTLLGNIT IFPHQFINLR SNNSATLILP YVNAVPMDSM LRHNNWSLVI |
| ef173417-64 | FDGTLLGNIT IFPHQFINLR SNNSATLILP YVNAVPMDSM LRHNNWSLVI |
| hrv-65 | CDGTLLGNIT IYPHQFINLR SNNSATLILP YVNAVPMDSM LRHNNWSIVI |
| hrv-66 | FDGTLLGNAL IFPHQFINLR SNNSATIIVP YVNAVPMDSM LRHNNWSLVI |
| hrv-67 | FDGTLLGNIT IFPHQFINLR SNNSATIIVP YVNAVPMDSM LRHNNWSLVI |
| hrv-68 | CDGTLLGNIT IYPHQYINLR SNNSATLILP YVNAVPMDSM VRHNNWSIVI |
| hrv-71 | CDGTLLGNIT IFPHQFINLR SNNSATLILP YVNAVPMDSM IRHNNWSIVI |
| dq473492-73 | FDGTLLGNST IFPHQFINLR SNNSATIIVP YVNAVPMDSM LRHNNWSLVI |
| dq473494-74 | FDGTLLGNIT IFPHQFINLR SNNSATLIVP YVNAVPMDSM LRHNNWSLVI |
| dq473510-75 | FDGTLLGNIT IFPHQFINLR SNNSATIIVP YVNAVPMDSM LRHNNWSLVI |
| dq473502-76 | FDGTLLGNIT VYPHQLINLR TNNSATIIVP YVNAVPMDSM VRHNNWSLVI |
| hrv-76 | FDGTLLGNIT VYPHQLINLR TNNSATIIVP YVNAVPMDSM VRHNNWSLVI |
| hrv-77 | FDGTLLGNAL IFPHQFINLR TNNSATIIVP YVNAVPMDSM LRHNNWSLVI |
| hrv-78 | CNGTLMGNLP IFPHQFINLR SNNSATLILP YMNAVPMDSM LRHNNWSIVI |
| ef173418-78 | CNGTLMGNLP IFPHQFINLR SNNSATLILP YMNAVPMDSM LRHNNWSIVI |
| hrv-80 | CNGTLLGNLT IFPHQFINLR SNNSATLILP YVNAVPMDSM LRHNNWSLVI |
| hrv-81 | FDGTLLGNLL IFPHQFINLR SNNSATIIVP YVNAVPMDSM VRHNNWSLVI |
| hrv-81-f06 | FDGTLLGNLL IFPHQFINLR SNNSATIIVP YVNAVPMDSM VRHNNWSLVI |
| hrv-81-f07 | FDGTLLGNLL IFPHQFINLR SNNSATIIVP YVNAVPMDSM VRHNNWSLVI |
| hrv-82 | FDGTLLGNIT IFPHQFINLR SNNSATLILP YVNAVPMDSM LRHNNWSLVI |
| dq473509-82 | FDGTLLGNIT IFPHQFINLR SNNSATLILP YVNAVPMDSM LRHNNWSLVI |
| hrv-85 | FDGTFLGNVL IFPHQFINLR SNNSATIIVP YVNAVPMDSM LRHNNWSLVI |
| pico-85 | FDGTFLGNVL IFPHQFINLR SNNSATIIVP YVNAVPMDSM LRHNNWSLVI |
| dq473504-88 | FDGTLLGNLP IYPHQYINLR TNNSATLILP YVNAVPMDSM LRHNNWSLVI |
| a10937-89 | FDGTLLGNLP IYPHQYINLR TNNSATLILP YVNAVPMDSM LRHNNWSLVI |
| hrv-89 | FDGTLLGNLP IYPHQYINLR TNNSATLILP YVNAVPMDSM LRHNNWSLVI |
| hrv-89-f08 | FDGTLLGNLP IYPHQYINLR TNNSATLILP YVNAVPMDSM LRHNNWSLVI |
| hrv-89-f09 | FDGTLLGNLP IYPHQYINLR TNNSATLILP YVNAVPMDSM LRHNNWSLVI |
| m16248-89 | FDGTLLGNLP IYPHQYINLR TNNSATLILP YVNAVPMDSM LRHNNWSLVI |
| hrv-90 | FDGTLLGNIT IFPHQFINLR SNNSATIIVP YVNAVPMDSM LRHNNWSLVI |
| hrv-94 | FDGTLLGNIT IFPHQFINLR SNNSATLILP YVNAVPMDSM LRHNNWSLVI |
| ef173419-94 | FDGTLLGNIT IFPHQYINLR SNNSATLILP YVNAVPMDSM LRHNNWSLVI |
| hrv-95 | CNGTLLGNAL IFPHQFINLR TNNAATLILP YVNATPMDSM LRHNNWSLLI |
| hrv-96 | FDGTLLGNLT IFPHQFINLR SNNSATIIVP YVNAVPMDSM LRHNNWSLVI |
| hrv-98 | FDGTFLGNLL IFPHQFINLR SNNSATIIVP YVNAVPMDSM LRHNNWSLLI |
| hrv-100 | FDGTLLGNAL IFPHQFINLR SNNSATIIVP YVNAVPMDSM LRHNNWSLVI |
| ef186077-qpm | CNGTLLGNLT IFPHQLINLR TNNSSTIVVP YINCTPMDSM LRHNNVSLVI |
| ef077279-nat001 | CNGTLLGNLT IFPHQLINLR TNNSSTIVVP YINCTPMDSM LRHNNVSLVI |
| ny1078 | CNGTLLGNLT IFPHQLINLR TNNSSSIVVP YINCTPMDSM LRHNNVSLVI |
| ef582385-c024 | CNGTLLGNIT IFPHQLINLR TNNSSTIVVP YINCVPMDNM LRHNNLSLVI |
| ef077280-nat045 | CNGTLLGNAL IYPHQMINLR TNNSATIVVP YINCVPMDNM LRHNNVSLLI |
| ef582387-c026 | CNGTLLGNLT IFPHQLINLR TNNSSTIVVP YINCTPMDSM LRHNNVSLVI |
| ef582386-c025 | CNGTLLGNLT IFPHQLINLR TNNSATIVVP YINCVPMDSM LRHNNISLVI |
| dq473485-03 | MDGTLLGNLL IFPHQFINLR TNNTATIVVP YINSVPIDSM TRHNNVSLMV |
| ef173422-03 | MDGTLLGNLL IFPHQFINLR TNNTATIVVP YINSVPIDSM TRHNNVSLMV |
| dq473490-04 | MDGTLIGNSL IFPHQFINLR TNNTATIVVP YINSVPMDSM TRHNNLSLMV |
| hrv-05 | MDGTLIGNSL IFPHQFINLR TNNTATIVVP YINSVPMDSM TRHNNLSLMV |
| dq473486-06 | MNGTLLGNLL IFPHQFINLR TNNTATIVVP YINSVPIDSM TRHNNVSLLV |
| l05355-14 | MNGTLLGNLL IFPHQFINLR TNNTATIVIP YINSVPIDSM TRHNNVSLMV |
| k02121-14 | MNGTLLGNLL IFPHQFINLR TNNTATIVIP YINSVPIDSM TRHNNVSLMV |
| ef173420-17 | MNGTLVGNLL IFPHQFINLR TNNTATIIVP YINSVPMDSM TRHNNASLLV |
| hrv-26 | MNGTLIGNSL IFPHQFINLR TNNTATIVVP YINSVPMDSM TRHNNLSLMV |
| hrv-27 | MDGTLLGNVL IFPHQFINLR TNNTATIVTP YINSVPMDSM TRHNNLSLLV |
| ef173421-27 | MDGTLLGNVL IFPHQFINLR TNNTATIVTP YINSVPMDSM TRHNNLSLLV |
| dq473487-35 | MDGTLLGNLL IFPHQFINLR TNNTATIIVP YMNSVPMDSM TRHNNVSLMV |
| hrv-35 | MDGTLLGNLL IFPHQFINLR TNNTATIIVP YMNSVPMDSM TRHNNVSLMV |
| ef173423-37 | MNGTLLGNLL IFPHQFINLR TNNTATIVVP YINSVPIDSM TRHNNVSLMV |
| hrv-42 | MDGTLIGNSL IFPHQFINLR TNNTATIVVP YINSVPMDSM TRHNNLSLLV |
| dq473488-48 | MNGTLVGNLL IFPHQFINLR TNNTATLVIP YINSVPMDSM TRHNNVSLLV |
| hrv-52 | MNGTLVGNLL IFPHQFINLR TNNTATLILP YINSVPMDSM TRHNNISLLV |
| hrv-52-f10 | MNGTLVGNLL IFPHQFINLR TNNTATLILP YINSVPMDSM TRHNNISLLV |
| ef173424-52 | MNGTLVGNLL IFPHQFINLR TNNTATLILP YINSVPMDSM TRHNNISLLV |
| hrv-69 | MNGTLVGNLL IFPHQFINLR TNNTATLIVP YINSVPMDSM TRHNNVSLLV |
| dq473489-70 | MNGTLIGNLL IFPHQFINLR TNNTATIIVP YINSVPMDSM TRHNNVSLLV |
| hrv-72 | MNGTLLGNLL IFPHQFINLR TNNTATLVVP YINSVPIDSM TRHNNVSLMI |
| hrv-79 | MDGTLLGNLL IFPHQFINLR TNNTATIIVP YMNSVPIDSM TRHNNISLMV |
| hrv-83 | MDGTLIGNLT IFPHQFINLR TNNTATIIVP YINSVPIDSM TRHNNISLMV |
| hrv-84 | MDGTLLGNAL IFPHQFINLR TNNTATLVMP YINSVPMDSM TRHNNLSLLV |
| hrv-86 | MNGTLLGNLL IFPHQFINLR TNNTATIVVP YINSVPIDSM TRHNNVSLLI |
| hrv-91 | MNGTLIGNLL IFPHQFINLR TNNTATIIVP YINSVPMDSM TRHNNLSLLV |
| hrv-92 | MDGTLLGNLT IFPHQFINLR TNNTATIVVP YINSVPIDSM TRHNNVSLMV |
| ef173425-93 | MDGTLLGNVL IFPHQFINLR TNNTATIVTP YINSAPMDSM TRHNNLSLLV |
| hrv-97 | MDGTLLGNVL IFPHQFINLR TNNTATIVTP YINSVPMDSM TRHNNLSLLV |
| hrv-99 | MNGTLIGNSL IFPHQFINLR TNNTATIVIP YINSVPMDSM TRHNNLSLMV |
| | 301 350 |
| annotation | BBBBBBBB.. ......BBBB BBBIBBBBBB BBB....... ....QG.... |
| af499637-cva13 | LPLSKLDYNP DAST.KLPIT VTIAPMCCEF NGLRNLTIP. ..ATQGLPVM |
| af546702-cva21 | LPLAPLAFAA TSSP.QVPIT VTIAPMCTEF NGLRNITVP. ..VHQGLPTM |
| v01149-pv1m | LPLAPLNFAS ESSP.EIPIT LTIAPMCCEF NGLRNITLP. ..RLQGLPVM |
| d00239-01 | IPISPLRSET TSSN.IRPIT VSISPMCAEF SGARAK.... .NVRQGLPVY |
| pico-01 | IPISPLRSET TSSN.IVPIT VSISPMCAKF SGARAK.... .NIKQGLPVY |
| hrv-01 | IPISPLRSET TSSN.IVPIT VSISPMCAEF SGARAK.... .NIKQGLPVY |
| x02316-02 | IPICPLE.TS SAIN.TIPIT ISISPMCAEF SGARAK.... ...RQGLPVF |
| hrv-07 | IPICPLQVES SGTP.TVPIT VSISPMFSEF SGPRNKVVF. ...TQGLPVM |
| dq473503-07 | IPICPLQVES SGIP.TVPIT VSISPMFSEF SGPRNKVVF. ...TQGLPVM |
| hrv-08 | VPISQLR... GDTN.SIPIT ISISPMAAEF SGARNR.... SARIEGLPVM |
| pico-09 | IPICPLQSSG TESS.TIPIT ISISPMFAEF SGARAK.... ...SQGLPVL |
| hrv-09 | IPICPLQSSG TESS.TXPIT ISISPMFAEF SGARAK.... ...SQGLPVL |
| hrv-09-f01 | IPICPLQSSG TESS.TIPIT ISISPMFAEF SGARAK.... ...SQGLPVL |
| hrv-09-f02 | IPICPLQSSG TESS.TIPIT ISISPMFAEF SGARAK.... ...SQGLPVL |
| dq473498-10 | IPISPLKSDP SASA.LVPIT ISISPMFAEF SGARAK...T AAANQGLPVY |
| hrv-10 | IPISPLKSDP SASA.LVPIT ISISPMFAEF SGARAK...T AAANQGLPVY |
| ef173414-11 | IPICPLESDL ..NT.PVPIT ISISPMCAEF SGARAR.... ...NQGLPVL |
| ef173415-12 | IPVCPLQVDA QATR.FVPIT ISISPMSAEF SGARN..... ATIPQGLPVM |
| hrv-13 | IPICELKCNN .TAV.TVPIT ISISPMCAEF SGARAR.... .NVTQGLPTL |
| hrv-13-f03 | IPICELKCNN .TAV.TVPIT ISISPMCAEF SGARAR.... .NVTQGLPTL |
| dq473493-15 | IPICQLEVVG GVSN.TVPIT ISISPMFAEF SGARAR.... ...SQGLPVM |
| l24917-16 | IPVCQLQSNN .ISN.IVPIT VSISPMCAEF SGARAK.... .TVVQGLPVY |
| hrv-18 | IPICQLE... GDPP.TIPIT ISISPMCAEF SGVRAR.... ...SQGLPAM |
| hrv-19 | IPICPLESQ. GTEG.VVPIT VSISPMFAEF SGARAR.... ...SQGLPVM |
| hrv-20 | IPISKIQFEN GATP.YVPIT LSISPMCSEF SGARN..... .NTTQGLPVM |
| hrv-21 | IPICPLESD. GQTP..VPIT ISISPMCAEF SGARAK.... ...SQGLPVM |
| hrv-22 | IPICPLEAS. GADV.TVPIT ISISPMFAEF SGARAR.... ...SQGLPVL |
| dq473497-23 | IPICPLETMS .TIT.TVPIT ISISPMCAEF SGARAR.... ...SQGLPVL |
| hrv-24 | IPICPLENST EVPN.AVPIT ISISPMCAEF SGARAR.... ...SQGLPVM |
| ef173416-24 | IPICPLENST EVPN.AVPIT ISISPMCAEF SGARAR.... ...SQGLPVM |
| hrv-25 | IPVSKLQADN .TSV.VVPIT ISISPMFAEF SGARAR..PT RAASQGLPTF |
| dq473508-28 | IPISKLETQ. GGTS.IVPIT ISISPMFSEF SGARN..... .NIPQGLPVM |
| hrv-29 | IPISELQAEN ATNV.TVPIT VSISPMFAEF SGARAR..PA RATTQGLPIY |
| dq473512-30 | IPICPLETMS .TIT.TVPIT ISISPMCAEF SGARAK.... ...SQGLPVL |
| hrv-30 | IPICPLETMS .TIT.TVPIT ISISPMCAEF SGARAK.... ...SQGLPVL |
| hrv-31 | IPICELLVDS TVNA.IVPIT ISISPMFAEF SGARAK...P EAASQGLPIY |
| hrv-32 | IPICPLQSEG TVST.TIPIT ISISPMFAEF SGARAK.... ...SQGLPVL |
| hrv-33 | IPICPLESVP ...ASTVPIT ISISPMCAEF SGARAA.... .ARNQGLPVF |
| hrv-34 | IPICQLE... GTPG.TVPIT ISISPMCAEF SGARAK.... ...NQGLPVM |
| dq473501-34 | IPICQLE... GTPG.TVPIT ISISPMCAEF SGARAK.... ...NQGLPVM |
| dq473505-36 | IPICPLQVQT GGTN.SIPIT VSISPMFSEF SGPRNKVVF. .GATEGLPVM |
| hrv-38 | IPICPLEVSS PGSN.SIPIT VSISPMFAEF SGARAR.... ...SQGLPTM |
| dq473495-38 | IPICPLEVSS PGSN.SIPIT VSISPMFAEF SGARAR.... ...SQGLPTM |
| ay751783-39 | IPVSPLEADT SATA.IVPIT VSISPMFSEF SGARAR...P AAATQGLPVY |
| hrv-40 | IPISPLKSDM SATS.KVPIT ISISPMCAEF SGARAR...P SATTQGLPVY |
| dq473491-41 | IPICELKCDT .TAA.TVPIT ISISPMCAEF SGARAR.... .NVTQGLPTL |
| hrv-43 | IPICELQVEG GVPR.TVPIT ISISPMYAEF SGARAK.... ...SQGIPTM |
| dq473499-44 | IPISELQTEN ATNV.TVPIT VSISPMFAEF SGARAR..PA RAAAQGLPIY |
| hrv-45 | VPVSELRGDT .....SIPIT VSISPMAAEF SGARNR.... SARVEGLPVM |
| dq473506-46 | IPVCPLEVAQ GSEQ.YVPIT ISISPMCSEF SGARN..... AARTQGLPVF |
| hrv-47 | IPICELLADS KASA.VVPIT ISISPMFAEF SGARAK...P VAAPQGLPIY |
| dq473496-49 | IPICPLETSS .TVN.TIPIT ISISPMCAEF SGARAK.... ...SQGLPVF |
| hrv-49-f04 | IPICPLETSS .TVN.TIPIT ISISPMCAEF SGARAK.... ...NQGLPVF |
| hrv-50 | IPICPLE... GTPT.TVPIT ISISPMCAEF SGARAK.... ...SQGLPVM |
| hrv-51 | IPVSPLASES ..TD.KVPIT ISISPMCSEF SGARNQ.... .STKQGLPVM |
| dq473507-53 | IPISKLVSV. GGTA.YVPIT ISISPMASEF SGARN..... NSPQGLPVM |
| hrv-54 | IPVSPLKSDQ .ASV.RVPIT ISISPMCAEF SGARAR...P AAAAQGLPVY |
| hrv-54-f05 | IPVSSLKSDQ .ASV.RVPIT ISISPMCAEF SGARAR...P AAAAQGLPVY |
| dq473511-55 | IPICPLEA.Q GVPN.TVPIT VSISPMCAEF SGARAK.... ...SQGLPVM |
| hrv-56 | IPVSPLKSDT LATS.QVPIT ISISPMCAEF SGARAK...P NAAPQGLPVY |
| hrv-57 | IPICPLEANG IQPV.VVPIT ISISPMCAEF SGARAK.... ...SQGLPVM |
| hrv-58 | IPICPLQVEG GGTP.SIPIT VSISPMFSEF SGPRNKVVF. ...SQGLPVM |
| dq473500-59 | IPVSPLKSDA SATS.KVPIT ISISPMFAEF SGARAK...P MAVGQGLPVY |
| hrv-60 | IPICPLEVST TGTN.GIPIT VSISPMFAEF SGARAR.... ...SQGLPTM |
| hrv-61 | IPICELRGS. GVDL.TIPIT ISISPMCAEF SGARAN.... ...SQGLPVL |
| hrv-62 | IPVSKLQADN TGSV.VVPIT ISISPMFAEF SGARAR..PA RATTEGLPTY |
| hrv-63 | IPISPLNSDP LATA.KVPIT ISISPMCAEF SGARAK...P VAVGQGLPVY |
| hrv-64 | IPICPLETES .VDT.TVPIT ISISPMYAEF SGARAR.... ...SQGLPVL |
| ef173417-64 | IPICPLETES .VDT.TVPIT ISISPMYAEF SGARAR.... ...SQGLPVL |
| hrv-65 | IPVSPLDSQA ..TG.NVPIT ISISPMCSEF SGARNQSV.. ...KQGLPVM |
| hrv-66 | IPVSPLKAEN ATSV.IVPIT ISISPMFAEF SGARAR...P VA..QGLPVY |
| hrv-67 | IPICPLQVEG AGSN.IIPIT VSISPMFAEF SGARAR.... ...NQGLPVL |
| hrv-68 | IPISQLKFEP GATP.FIPIT LSISPMCSEF SGARNN.... ..TTQGLPVM |
| hrv-71 | IPISPLEAN. .VTD.PVPIT ISISPMCTEF SGARNQSV.. ..KIQGLPVM |
| dq473492-73 | IPICKLRCQ. GISP.VVPIT ISISPMCAEF SGARAR.... .NVTQGLPTF |
| dq473494-74 | IPICPLEVSG NEVN.TVPIT ISISPMFAEF SGARAR.... ...SQGLPVM |
| dq473510-75 | IPICELQVDT GVPA.TVPIT ISISPMFAEF SGARAR.... ...SQGIPTM |
| dq473502-76 | IPICPLESLP ...SSTVPIT ISISPMCAEF SGARA..... TARNQGLPVF |
| hrv-76 | IPICPLESLP ...SSTVPIT ISISPMCAEF SGARA..... TARNQGLPVF |
| hrv-77 | IPISPLKSDT AATA.IVPIT ISISPMFAEF SGARAH...P TA..QGLPVY |
| hrv-78 | IPVCSLQSEA GATG.LVPIT VSISPMFAEF SGARN.AVV. ...QQGLPVM |
| ef173418-78 | IPVCSLQSEA GATG.LVPIT VSISPMFAEF SGARN.AVV. ...QQGLPVM |
| hrv-80 | IPVCPLEVAV GSEQ.YVPIT ISISPMCSEF SGARN..MV. ....QGLPVF |
| hrv-81 | IPICQLQSNN .TSN.IVPIT VSISPMFAEF SGARAKTVV. ....QGLPVY |
| hrv-81-f06 | IPICQLQSNN .TSN.IVPIT VSISPMFAEF SGARAKTVV. ....QGLPVY |
| hrv-81-f07 | IPICQLQSNN .TSN.IVPIT VSISPMFAEF SGARAKTVV. ....QGLPVY |
| hrv-82 | IPICPLETN. GGDA.TVPIT ISISPMFAEF SGARAR.... ...SQGLPVL |
| dq473509-82 | IPICPLETN. GGDV.TVPIT ISISPMFAEF SGARAR.... ...SQGLPVL |
| hrv-85 | IPISPLKSDT TATS.KVPIT ISISPMCAEF SGARAR...P VAAAQGLPVY |
| pico-85 | IPISPLKSDT TATS.KVPIT ISISPMCAEF SGARAR...P VAAAQGLPVY |
| dq473504-88 | IPICPLQVEM SGTQ.TVPIT VSISPMFSEF SGPRNKVVF. ...NQGFPVM |
| a10937-89 | IPICPLQVQP GGTQ.SIPIT VSISPMFSEF SGPRSKVVF. .STTQGLPVM |
| hrv-89 | IPICPLQVQP GGTQ.SIPIT VSISPMFSEF SGPRNKVVF. .STTQGLPVM |
| hrv-89-f08 | IPICPLQVQP GGTQ.SIPIT VSISPMFSEF SGPRNKVVF. .STTQGLPVM |
| hrv-89-f09 | IPICPLQVQP GGTQ.SIPIT VSISPMFSEF SGPRNKVVF. .STTQGLPVM |
| m16248-89 | IPICPLQVQP GGTQ.SIPIT VSISPMFSEF SGPRSKVVF. .STTQGLPVM |
| hrv-90 | IPICPLESMQ .VPN.TVPIT ISISPMCAEF SGARAR.... ...SQGLPVM |
| hrv-94 | IPICPLETQS .QDT.TVPIT ISISPMYAEF SGARAR.... ...SQGLPVL |
| ef173419-94 | IPICPLETQS .QDT.TVPIT ISISPMYAEF SGARAR.... ...SQGLPVL |
| hrv-95 | VPISQLR... GDTN.SIPIT ISISPMAAEF SGARNR.... SARIEGLPVM |
| hrv-96 | IPICRLNSM. GGEP.TVPIT ISISPMCAEF SGARAK.... ...NQGLPVL |
| hrv-98 | IPVSPLKSDQ .ASI.RVPIT ISISPMCAEF SGARAK...P TAAAQGLPVY |
| hrv-100 | IPISPLKSEP GASA.LVPIT ISISPMFAEF SGARAK...T IAAPQGLPVY |
| ef186077-qpm | IPICPLRAPA TAPS.TLPIT ISIAPIKSEF SGARQS.... .AKAQGLPVR |
| ef077279-nat001 | IPICPLRPPN PGQQ.TLPIT ISIAPIKSEF SGARQA...I KATAQGLPVR |
| ny1078 | IPICPLKTPS GAPR.TLPIT VSIAPDRSES SGARQS.... .ATRQGLPTR |
| ef582385-c024 | IPIVPLRAAN GVTK..VPIT ISIAPDKSEF SGARQS.... ..VKQGLPTR |
| ef077280-nat045 | IPIVPLKANT DAVN.SLPIT VTIAPDKSEF SGAMKS.... ..QQQGLPTR |
| ef582387-c026 | IPICPLRAPA TAPQ.TLPIT ISIAPIKSEF SGARQS.... ..TKQGLPVR |
| ef582386-c025 | IPLVPLRSGS TQTINRLPIT ISIAPDKSEF SGARQS.... .NKVQGLPVR |
| dq473485-03 | VPIAPLNAPT GSSP.TLPVT VTIAPMCTEF TGIRSRSIVP ....QGLPTT |
| ef173422-03 | VPIAPLNAPT GSSP.TLPVT VTIAPMCTEF TGIRSRSIVP ....QGLPTT |
| dq473490-04 | IPIVNITATS GAAP.SISIT VTIAPMYLEL SGIRSKTVI. ....QGLPTV |
| hrv-05 | IPIVDITATS GTTP.SIPVT ITIAPMFLEL SGIRSKAVI. ....QGLPAV |
| dq473486-06 | IPIAPLVAPT GSPQ.TLPVT VTIAPMCTEF TGIRSKTIVP ....QGLPTT |
| l05355-14 | IPIAPLTVPT GATP.SLPIT VTIAPMCTEF SGIRSKSIVP ....QGLPTT |
| k02121-14 | IPIAPLTVPT GATP.SLPIT VTIAPMCTEF SGIRSKSIVP ....QGLPTT |
| ef173420-17 | IPVVPLVNPV GMST.IIPIT VTIAPMYTEF SGIRSKAVVP ....QGLPTL |
| hrv-26 | IPIVGITATS GSAP.SIPVT VTIAPMFLEL SGIRSRTVI. ....QGLPTV |
| hrv-27 | IPVVPITANT GSPP.SIPVT VTIAPMNLEL SGIRSKTVV. ..KEQGLPTT |
| ef173421-27 | IPVVPITANT GSPP.SIPVT VTIAPMNLEL SGIRSKTVV. ..KEQGLPTT |
| dq473487-35 | IPIAPLAAPT GSAT.TLPIT VTIAPMFTEF TGIRSKTIVP NATPQGLPTT |
| hrv-35 | IPIAPLAAPT GSAT.TLPIT VTIAPMFTEF TGIRSKTIVP NATPQGLPTT |
| ef173423-37 | IPIAPLVAPT GSPQ.TLPVT VTIAPMCTEF TGIRSKTIVP ....QGLPTT |
| hrv-42 | IPIVQIAATT GAAP.SIPVT VTIAPMFLEL SGIRSKAVI. ....QGLPTV |
| dq473488-48 | IPIVPLAHPT GMTT.TIPIT VTIAPMCTEF SGIRSKAVAI ....QGLPTL |
| hrv-52 | IPIVPLSNPT GMTT.TIPVT VTIAPMYTEF SGIRSKAVVP ....QGLPTL |
| hrv-52-f10 | IPIVPLSSPS GMTT.TIPVT VTIAPMYTEF SGIRSKAVI. ...KQGLPTL |
| ef173424-52 | IPIVPLSNPT GMTT.TIPVT VTIAPMYTEF SGIRSKAVVP ....QGLPTL |
| hrv-69 | IPVVPLTNPT GMST.TIPIT VTIAPMNTEF SGIRSRAVVP ....QGLPTL |
| dq473489-70 | IPVVPLVNPV GMST.IIPVT VTIAPMYTEF SGIRSKAVVP ....QGLPTL |
| hrv-72 | IPIAPLIVPT NASP.TLPVT VTIAPMCTEF TGIRSKTVV. ...PQGLPTT |
| hrv-79 | IPISPLMAPT GNSQ.TLPIT VTIAPMCTEF TGIRSKTIVP NVSPQGLPVT |
| hrv-83 | IPIVPLSVPN GNQP.TLPIT VTIAPMCTEF SGIRSKTIVP NASPQGLPTT |
| hrv-84 | IPVIPITATS GAAH.SVPIT VTIAPMFLEL SGIRSMAVV. ...TQGLPTT |
| hrv-86 | IPISPLTAPT NSPN.TLPVT VTIAPMCAEF SGIRSRSIVP ....QGLPTT |
| hrv-91 | IPVVPLINPV GMST.IIPIT VTIAPMCTEF SGIRSQAV.. ..KIQGLPTL |
| hrv-92 | IPIVPLMAPT GNAT.TLPIT VTIAPMCTEF SGIRSKTIVP NATPQGLPTT |
| ef173425-93 | IPVVPITAST GSAP.SIPVT VTIAPMNPEL SGIRSKTVI. ..TPQGLPTT |
| hrv-97 | IPIVPITAST GSSH.SIPIT VTIAPMNLEL SGIRSKTVI. ...TQGLPTT |
| hrv-99 | IPVVNITATS GTSP.SIPVT VTIAPMFLEL SGIRSKAVI. ....QGLPTV |
| | 351 400 |
| annotation | .......... .......... .......... .....BBBBB BBBBBBBBKK |
| af499637-cva13 | STPGSNQYLT SDNFQSPCAL PEFDVTQPIF IPGEVKNLME LAEIDTMIPM |
| af546702-cva21 | NTPGSNQFLT SDDFQSPCAL PNFDVTPPIH IPGEVKNMME LAEIDTLIPM |
| v01149-pv1m | NTPGSNQYLT ADNFQSPCAL PEFDVTPPID IPGEVKNMME LAEIDTMIPF |
| d00239-01 | ITPGSGQFMT TDDMQSPCAL PWYHPTKEIS IPGEVKNLIE MCQVDTLIPV |
| pico-01 | ITPGSGQFMT TDDMQSPCAL PWYHPTKEIS IPGEVKNLIE MCQVDTLIPV |
| hrv-01 | ITPGSGQFMT TDDMQSPCAL PWYHPTKEIS IPGEVKNLIE MCQVDTLIPV |
| x02316-02 | ITPGSGQFLT TDDFQSPCAL PWYHPTKEIS IPGEVKNLVE ICQVDSLVPI |
| hrv-07 | LTPGSGQFLT TDDTQSPSAF PYFHPTKEIF IPGQVRNLIE MCQVDTLIPI |
| dq473503-07 | LTPGSGQFLT TDDTQSPSAF PYFHPTKEIF IPGQVRNLIE MCQVDTLIPI |
| hrv-08 | LTPGSGQFLT TDDMQSPSVL PYFHPTQEIF IPGEVKNLVE LCQVDTMVPL |
| pico-09 | TTPGSGQFMT TDDFQSPSAL PWFHPTKEIS IPGQVRNLIE MCQVDTIIPI |
| hrv-09 | TTPGSGQFMT TDDFQSPSAL PWFHPTKEIS IPGQVRNLIE MCQVDTIIPI |
| hrv-09-f01 | TTPGSGQFMT TDDFQSPSAL PWFHPTKEIS IPGQVRNLIE MCQVDTIIPI |
| hrv-09-f02 | TTPGSGQFMT TDDFQSPSAL PWFHPTKEIS IPGQVRNLIE MCQVDTIIPI |
| dq473498-10 | VTPGSGQFMT TDDLQSPSAL PWYHPTKEIL IPGEVKNLIE MCQVDSLIPI |
| hrv-10 | VTPGSGQFMT TDDLQSPSAL PWYHPTKEIL IPGEVKNLIE MCQVDSLIPI |
| ef173414-11 | ITPGSGQFMT TDDFQSPSAL PWFHPTKEIS IPGQVRNLIE LCQVDTLIPI |
| ef173415-12 | ITPGSGQFLT TDDAQSPSAL PYFHPTKEIF IPGQVKNLIE MCQVDTLIPV |
| hrv-13 | LTPGSGQFLT TDDFQSPSAL PWYHPTKEIS IPGQVRNLIE LCQVDTMIPI |
| hrv-13-f03 | LTPGSGQFLT TDDFQSPSAL PWYHPTKEIS IPGQVRNLIE LCQVDTMIPI |
| dq473493-15 | MTPGSGQFMT TDDYQSPSAL PWFHPTKEIS IPGEVKNLVE MCQVDTLIPI |
| l24917-16 | VTPGSGQFMT TDDMQSPCAL PWYHPTKEIF IPGEVKNLIE MCQVDTLIPI |
| hrv-18 | LTPGSGQFMT TDDFQSPCAL PWFHPTKEIS IPGQVRNLIE LCQVDTLIPI |
| hrv-19 | ITPGSGQFMT TDDYQSPSAL PWFHSTKEIS IPGQVRNLAE LCQVDTIIPI |
| hrv-20 | LTPGSAQFLT TDDAQSPSAL PYFHPTKEIF IPGKVNNLSE ICQVDSMIPI |
| hrv-21 | LTPGSGQFMT TDDFQSPSAL PWFHPTKEIS IPGQVTNLIE LCQVDTLIPV |
| hrv-22 | LTPGSGQFLT TDDYQSPSAL PWFHPTKEIF IPGQVRNLSE LCQVDTIIPI |
| dq473497-23 | ITPGSGQFLT TDDFQSPCAL PWYHPTKEIF IPGEVKNLVE ICQVDSLIPI |
| hrv-24 | ITPGSGQFLT TDDFQSPSAL PWFHPTKEIS IPGQVRNLVE LCQVDTLIPI |
| ef173416-24 | ITPGSGQFLT TDDFQSPSAL PWFHPTKEIS IPGQVRNLVE LCQVDTLIPI |
| hrv-25 | ITPGSGQFMT TDDFQSPSAL PWYHPTKEIL IPGEVKNLIE MCQVDTLIPI |
| dq473508-28 | LTPGSAQFLT TDDAQSPSAL PYFHPTKEIF IPGRVNNLSE ICQIDSMIPI |
| hrv-29 | MTPGSGQFMT TDDFQSPSAL PWYHPTKEIL IPGEVKNLIE ICQVDTLIPI |
| dq473512-30 | VTPGSGQFLT TDDYQSPCAL PWYHPTKEIF IPGEVKNLVE ICQVDSLIPI |
| hrv-30 | VTPGSGQFLT TDDYQSPCAL PWYHPTKEIF IPGEVKNLVE ICQVDSLIPI |
| hrv-31 | MTPGSGQFMT TDDFQSPSAL PWYHPTKEIL IPGEVKNLIE MCQVDTLIPI |
| hrv-32 | MTPGSGQFMT TDDFQSPSAL PWFHPTKEIS IPGQVRNLIE MCQVDTIIPI |
| hrv-33 | ITPGSGQFMT TDDFQSPSAL PWFHPTREIS IPGQVKNLVE LCQIDTLIPI |
| hrv-34 | LTPGSGQFMT TDDFQSPCAL PWYHPTKEIS IPGQVRNLIE LCQVDTLIPI |
| dq473501-34 | LTPGSGQFMT TDDFQSPCAL PWYHPTKEIS IPGQVRNLIE LCQVDTLIPI |
| dq473505-36 | LTPGSGQFLT TDDTQSPSAF PYFHPTKEIF IPGQVRNLIE MCQVDTLIPV |
| hrv-38 | MTPGSGQFMT TDDFQSPCAL PWFHPTKEIS IPGEVKNLIE LCQVDTMIPI |
| dq473495-38 | MTPGSGQFMT TDDFQSPCAL PWFHPTKEIS IPGEVKNLIE LCQVDTMIPI |
| ay751783-39 | MTPGSGQFLT TDDLQSPSAL PWYHPTKEIF IPGQVRNLIE MCQVDTMIPI |
| hrv-40 | LTPGSNQFMT TDDFQSPCAL PWYHPTKEIF IPGEVKNLIE MCQVDTLIPI |
| dq473491-41 | LTPGSGQFLT TDDYQSPSAL PWYHPTKEIS IPGQVQNLIE LCQVATMIPI |
| hrv-43 | LTPGSGQFMT TDDFQSPSAL PWYHPTKEIS IPGQVRNLIE MCEVDTLIPI |
| dq473499-44 | MTPGSGQFMT TDDFQSPSAL PWYHPTKEIL IPGEVKNLME ICQVDTLIPI |
| hrv-45 | LTPGSGQFLT TDDMQSPSVL PYFHSTQEIF IPGEVKNLIE LCQVDTMVPL |
| dq473506-46 | ITPGSAQFLT TDDTQSPSAL PYFHPTKEIF IPGEVKNLSE ICQVDSMIPI |
| hrv-47 | MTPGSGQFMT TDDFQSPSAL PWYHPTKEIL IPGEVRNLIE MCQVDTLIPI |
| dq473496-49 | ITPGSGQFLT TDDFQSPCAL PWYHPTKEIS IPGEVKNLVE ICQVDSLVPI |
| hrv-49-f04 | ITPGSGQFLT TDDFQSPCAL PWYHPTKEIS IPGEVKNLVE ICQVDSLVPI |
| hrv-50 | LTPGSGQFMT TDDFQSPCAL PWYHPTKEIS IPGQVRNLIE LCQVDTLIPI |
| hrv-51 | LTPGSAQFLT TDDTQSPSAL PYFHPTKEIF IPGKVSNLSE ICQVDSMIPV |
| dq473507-53 | LTPGSEQFLT TDDAQSPSAL PYFHPTKEIF IPGRVTNLSE ICQIDSMIPI |
| hrv-54 | VTPGSGQFMT TDDLQSPCAL PWYHPTKEIF IPGEVRNLIE MCQVDTLIPI |
| hrv-54-f05 | ITPGSGQFMT TDDLQSPCAL PWYHPTKEIF IPGEVRNLIE MCQVDTLIPI |
| dq473511-55 | TTPGSGQFMT TDDYQSPCAL PWFHSTKEIS IPGQVRNLIE LCQVDTLIPV |
| hrv-56 | VTPGSGQFMT TDDLQSPSAL PWYHPTKEIF IPGEVKNLIE MCQVGTLIPV |
| hrv-57 | VTPGSGQFMT TDDYQSPSAL PWFHPTKEIS IPGQVRNLIE MCQVDTLIPV |
| hrv-58 | LTPGSGQFLT TDDTQSPSAF PYFHPTKEIF IPGQVKNLIE MCQVDTLIPI |
| dq473500-59 | ITPGSGQFMT TDDLQSPSAL PWYHPTKEIF IPGEVKNLIE MCQVDTLIPI |
| hrv-60 | LTPGSGQFMT TDDFQSPCAL PWFHPTKEIS IPGEVKNLIE LCQVDSMIPI |
| hrv-61 | LTPGSGQFMT TDDFQSPSAL PWYHPTKEIS IPGQVRNLVE LCQVDTLIPI |
| hrv-62 | ITPGSGQFMT TDDFQSPSAL PWYHPTKEIL IPGEVKNLIE MCQVDTLIPI |
| hrv-63 | VTPGSGQFMT TDDLQSPSAL PWYHSTKEIF IPGEVKNLIE MCQVGTLIPV |
| hrv-64 | LTPGSGQFMT TDDYQSPSAL PWFHPTKEIF IPGQVQNLSE LCQVDTIIPI |
| ef173417-64 | LTPGSGQFMT TDDYQSPSAL PWFHPTKEIF IPGQVQNLSE LCQVDTIIPI |
| hrv-65 | LTPGSAQFLT TDDTQSPSAL PYFHSTKEIF IPGKVNNLSE ICQVDSMIPI |
| hrv-66 | ITPGSGQFMT TDDLQSPSAL PWYHPTKEIM IPGEVKNLIE MCQVDTLIPI |
| hrv-67 | TTPGSGQFMT TDDFQSPSAL PWFHPTKEIS IPGQVRNLIE ICQVDTLIPI |
| hrv-68 | LTPGSAQFLT TDDAQSPSAL PYFHPTKKIF IPGEVKNLSE ICQVDSMIPI |
| hrv-71 | LTPGSAQFLT TDDAQSPSAL PYFHPTKEIF IPGEVKNLSE ICQVDSIIPI |
| dq473492-73 | ITPGSGQFLT TDDFQSPSAL PWYHPTKEIS IPGQVRNLVE LCQVDTMIPI |
| dq473494-74 | MTPGSGQFMT TDDFQSPSAL PWFHPTKEIS IPGQVRNLVE MCQVDTLIPI |
| dq473510-75 | LTPGSGQFMT TDDFQSPSAL PWYHPTKEIS IPGQVRNLIE LCEVDTLIPI |
| dq473502-76 | ITPGSGQFMT TDDFQSPSAL PWFHPTREIS IPGQVKNFVE LCQIDTLIPI |
| hrv-76 | ITPGSGQFMT TDDFQSPSAL PWFHPTREIS IPGQVKNLVE LCQIDTLIPI |
| hrv-77 | ITPGSGQFMT TDDLQSPSAL PWYHPTKEIM IPGEVKNLIE MCQVDTLIPI |
| hrv-78 | LTPGSEQFLT TDDNQSPSAL PWFHPSKEIF IPGRVTNLAE ICQVDTLIPV |
| ef173418-78 | LTPGSEQFLT TDDNQSPSAL PWFHPSKEIF IPGRVTNLAE ICQVDTLIPV |
| hrv-80 | LTPGSAQFLT TDDTQSPSAL PYFHPTKEIF IPGEVKNLSE ICQVDSMIPV |
| hrv-81 | VTPGSGQFMT TDDMQSPCAL PWYHPTKEIF IPGEVKNLIE MCQVDTLIPI |
| hrv-81-f06 | VTPGSGQFMT TDDMQSPCAL PWYHPTKEIF IPGEVKNLIE MCQVDTLIPI |
| hrv-81-f07 | VTPGSGQFMT TDDMQSPCAL PWYHPTKEIF IPGEVKNLIE MCQVDTLIPI |
| hrv-82 | MTPGSGQFMT TDDYQSPSAL PWFRSTKEIF IPGQVHNLAE LCQVDTIIPI |
| dq473509-82 | MTPGSGQFMT TDDYQSPSAL PWFHSTKEIF IPGQVHNLAE LCQVDTIIPI |
| hrv-85 | LTPGSNQFMT TDDFQSPCAL PWYHPTKEIF IPGEVKNLIE MCQVDTLIPI |
| pico-85 | LTPGSNQFMT TDDFQSPCAL PWYHPTKEIF IPGEVKNLIE MCQVDTLIPI |
| dq473504-88 | LTPGSGQFLT TDDMQSPSAF PYFHPTKEIF IPGEVKNLVE MCQVDTLIPV |
| a10937-89 | LTPGSGQFLT TDDTQSPSAF PYFHPTKEIF IPGQVRNLIE MCQVDTLIPV |
| hrv-89 | LTPGSGQFLT TDDTQSPSAF PYFHPTKEIF IPGQVRNLIE MCQVXTLIPV |
| hrv-89-f08 | LTPGSGQFLT TDDTQSPSAF PYFHPTKEIF IPGQVRNLIE MCQVDTLIPV |
| hrv-89-f09 | LTPGSGQFLT TDDTQSPSAF PYFHPTKEIF IPGQVRNLIE MCQVDTLIPV |
| m16248-89 | LTPGSGQFLT TDDTQSPSAF PYFHPTKEIF IPGQVRNLIE MCQVDTLIPV |
| hrv-90 | ITPGSGQFLT TDDFQSPSAL PWFHPTKEIS IPGQVRNLVE LCQVDTLIPI |
| hrv-94 | MTPGSGQFMT TDDYQSPSAL PWFHPTKEIF IPGQVQNLSE LCQVDTIIPI |
| ef173419-94 | MTPGSGQFMT TDDYQSPSAL PWFHPTKEIF IPGQVQNLSE LCQVDTIIPI |
| hrv-95 | LTPGSGQFLT TDDMQSPSVL PYFHPTQEIF IPGEVKNLVE LCQVDTMVPL |
| hrv-96 | LTPGSGQFMT TDDFQSPSAL PWYHPTKEIS IPGQVRNLIE LCQVDTLIPI |
| hrv-98 | VTPGSGQFMT TDDLQSPCAL PWYHPTKEIF IPGEVKNLIE MCQVDTLIPV |
| hrv-100 | ITPGSGQFLT TDDLQSPSAL PWYHPTKEIL IPGEVKNLIE MCQVDTLIPI |
| ef186077-qpm | MPSGANQFMT TEDEQSPNIL PSFHSTKQIH IPGEIKTIMD MARVDSFMPI |
| ef077279-nat001 | MPSGANQFMT TEDEQSPNVL PGFHPTKQIH IPGEIKTIMD IARVDSFMPI |
| ny1078 | MPSGANQFMT TEDEQSGNIL PNFSPTKQIH IPGQIKTIMD MARVDSFMPI |
| ef582385-c024 | LPSGSQQFLT TEDEQSANLL PDFSVTKIID IPGRITNILH MAMVDSFMPL |
| ef077280-nat045 | SPAGSQQFMT TEDEQSPNIL PEYSPTKMIH IPGRIDNILH IAMVESLIPL |
| ef582387-c026 | MPSGANQFMT TEDEQSPNIL PSFHSTKQIH IPGQIKTIMD MARVDSFMPI |
| ef582386-c025 | MPSGAQQFMT TEDEQSANLL PGFHPTPIMH IPGEITNIMH MARVDSFMPI |
| dq473485-03 | TLPGSGQFLT TDDRQSPSAL PSYEPTPRIH IPGKVRNLLE IIQVGTLIPM |
| et173422-03 | TLPGSGQFLT TDDRQSPSAL PSYEPTPRIH IPGKVRNLLE IIQVGTLIPM |
| dq473490-04 | LTPGSEQFLT TDDRQSPSAM PNYEPTPLIH IPGEVRNLLE IAQVDTLIPI |
| hrv-05 | LTPGSEQFLT TDDRQSPSAM PNYEPTPLIH IPGEVKNLLE IAQVDTLIPL |
| dq473486-06 | TLPGSGQFLT TDDRQSPSAL PNYEPTPRIH IPGKVRNLLE IIQVDTLIPM |
| l05355-14 | TLPGSGQFLT TDDRQSPSAL PNYEPTPRIH IPGKVHNLLE IIQVDTLIPM |
| k02121-14 | TLPGSGQFLT TDDRQSPSAL PNYEPTPRIH ILGKVHNLLE IIQVDTLIPM |
| ef173420-17 | TTPGSGQFLT TDDRQSPSAM PNYEPTPLIH IPGEVHNLLE IIQVDTLIPM |
| hrv-26 | LTPGSEQFLT TDDRQSPSAM PNYEPTPLIH IPGEVNNLLE MAQVDTLIPI |
| hrv-27 | LTPGSEQFLT TDDRQSPSAL PNYEPTPLIH IPGRVTNLLE LVQVDTMIPI |
| ef173421-27 | LTPGSEQFLT TDDRQSPSAL PNYEPTPLIH IPGRVTNLLE LVQVDTMIPI |
| dq473487-35 | TLPGSGQFLT TDDRQSPSAL PNYEPTPKIH IPGEVKNLLE IIQVDTLIPM |
| hrv-35 | TLPGSGQFLT TDDRQSPSAL PNYEPTPKIH IPGEVKNLLE IIQVDTLIPM |
| ef173423-37 | TLPGSGQFLT TDDRQSPSAL PSYEPTPRIH IPGKVENLLE IIQVDTLIPM |
| hrv-42 | LTPGSEQFLT TDDRQSPSAM PNYEPTPLIH IPGEVKNLLE IAQVDTLIPI |
| dq473488-48 | TTPGSGQFLT TDDRQSPSAM PNYEPTPPIH IPGEVHNLLE IIQVDTLIPM |
| hrv-52 | TTPGSGQFLT TDDRQSPSAM PNYEPTPQIH IPGEVHNLLE LIQVDTLIPL |
| hrv-52-f10 | TTPGSGQFLT TDDRQSPSAM PNYEPTPQIH IPGEVHNLLE LIQVDTLIPL |
| ef173424-52 | TTPGSGQFLT TDDRQSPSAM PNYEPTPQIH IPGEVHNLLE LIQVDTLIPL |
| hrv-69 | TTPGSGQFLT TDDRQSPSAM PNYEPTPPIH IPGEVHNLLE IIQVDTLIPM |
| dq473489-70 | TTPGSGQFLT TDDRQSPSAM PNYEPTPLIH IPGEVHNLLE IIQVDTLIPM |
| hrv-72 | TLPGSGQFLT TDDRQSPSAL PNYEPTPKIH IPGQVKNLLE IIQVDTLIPM |
| hrv-79 | TLPGSGQFLT TDDRQSPSAL PNYEPTPRIH IPGKVRNLLE IIQVDTLIPM |
| hrv-83 | TLPGSGQFLT TDDRQSPSAL PNYEPTPRIH IPGQVRNLLE IIQVDTMIPM |
| hrv-84 | STPGSNQFLT TDDRQSPSAL PNYEVTPLIH IPGRVVNLLE IAQVDSLIPI |
| hrv-86 | TLPGSGQFLT TDDRQSPSAL PCYEPTPKIH IPGEVKNLLE MIQVDTLIPM |
| hrv-91 | TTPGSGQFLT TDDRQSPSAM PNYEPTPPIH IPGEVHNLLE IIQVDTLIPM |
| hrv-92 | TLPGSGQFLT TDDRQSPSAL PNYEPTPRIH IPGQVKNLLE IIQVDTLIPM |
| ef173425-93 | LTPGSEQFLT TDDRQSPSAL PNYEPTPLIH IPGRVTNLLE LIQVDTMIPI |
| hrv-97 | LTPGSEQFLT TDDRQSPSAL PNYEPTPLIH IPGRVTNLLE IIQVDSMIPI |
| hrv-99 | LTPGSEQFLT TDDRQSPSAM PNYEPTPLIH IPGEVKNLLE IAQVDTLIPI |
| | 401 450 |
| annotation | KKKKKK..KK KKKKK..... .......... BBCBBHHHHH H.HHAHHHHH |
| af499637-cva13 | DLSEGK.RNS MDMYRVKISD AGDR..NKPI LCLTLSPASD PRLSYTMLGE |
| af546702-cva21 | NAVDGK.VNT MEMYQIPLND NLSK...API FCLSLSPASD KRLSHTMLGE |
| v01149-pv1m | DLSATK.KNT MEMYRVRLSD KPHT..DDPI LCLSLSPASD PRLSHTMLGE |
| d00239-01 | NNVGTN.VGN ISMYTVQLGN QMDM..AQEV FAIKVDITSQ P.LATTLIGE |
| pico-01 | NNVGNN.VGN VSMYTVQLGN QTGM..AQKV FSIKVDITSQ P.LATTLIGE |
| hrv-01 | NNVGNN.VGN VSMYTVQLGN QTGM..AQKV FSIKVDITSQ P.LATTLIGE |
| x02316-02 | NNTDTY.INS ENMYSVVLQS SINA..PDKI FSIRTDVASQ P.LATTLIGE |
| hrv-07 | NNTGNN.VRS ANMYTVDLTT QDHP.YAQEV FAIPVDVASH P.LATTYLGE |
| dq473503-07 | NNTGNN.VRS ANMYTVDLTT QDHP.YAQEV FAIPVDVASH P.LATTYLGE |
| hrv-08 | NNINAS.RNR IDMYALPLTR QPQS..AVKL FTIPIDIASP P.LNTTLLGE |
| pico-09 | NNTGTN.TTS INMYTVQLER QADL..MTEV FAINVDIASH P.LATTMLGE |
| hrv-09 | NNTGTN.TTS INMYTVQVER QADL..MSEV FAINVDIASH P.LATTMLGE |
| hrv-09-f01 | NNTGTN.TTS INMYTVQLER TADL..MSEV FAINVDIASH P.LATTMLGE |
| hrv-09-f02 | NNTGTN.TTS INMYTVQLER TADL..MSEV FAINVDIASH P.LATTMLGE |
| dq473498-10 | NNTTAN.LRK VNMYTIQLAN QTNM..AEEI FAIKVDIASQ P.LSNTIIGE |
| hrv-10 | NNTTAN.LRK VNMYTIQLAN QTNM..AEEI FAIKVDIASQ P.LSNTIIGE |
| ef173414-11 | NSVEGN.TDN TSIYTVTLTR ETNL..AQEI FAIKVDIASH P.LATTLIGE |
| ef173415-12 | NNVQAS.KNN ISMYTVQLTT QTDV..AKEV FAIPVDVASQ P.LATTMLGE |
| hrv-13 | NNTNDN.IRD KSMYTIRLGG VTNA..KEAI FAMRVDIASQ P.LATTMIGE |
| hrv-13-f03 | NNTNDT.IRD KSMYTIRLGG VTNA..KEAI FAMRVDIASQ P.LATTMIGE |
| dq473493-15 | NNTPGN.VNT INMYTVLLER KDDL..MQEV FSIRVDIASQ P.LATTMIGE |
| l24917-16 | NSTQSN.IGN VSMYTVTLSP QTKL..AEEI FAIKVDIASH P.LATTLIGE |
| hrv-18 | NNLQAN.ITN LDMYSVVLNR EIDP..AKEV FAIKVDIASQ P.LATTLIGE |
| hrv-19 | NHTTST.TSS VNMYTVVLDK KTSL..AEEV FAIKVDIASQ P.LATTLIGE |
| hrv-20 | NNTIAV.KNS AGMYTVQVSQ QTQP..AQEI FSILVDVASQ P.IATTLIGE |
| hrv-21 | NNTETNGVTN INMYTVTINR EVNITPAKEI FAIKVDIASQ P.LATTLIGE |
| hrv-22 | NHTTST.TSS VNMYTIQLTK QQPG...TEV FAMRVDVASQ P.LATTLIGE |
| dq473497-23 | NNLEGN.ITS EAMYSIELQS STEQ..SK.I FSIRTDIASQ P.LATTLIGE |
| hrv-24 | DSVEAN.IKS TNMYTVTLTR ETDS..AKEI FAIKVDIASH P.LATTLIGE |
| ef173416-24 | DSVEAN.IKS TNMYTVTLTR ETDS..AKEI FAIKVDIASH P.LATTLIGE |
| hrv-25 | NNTNNN.LRK VNLYTVELTN QPMP..AQKI FSIKVDIASQ P.LVNTMLGQ |
| dq473508-28 | NNTHAV.KNT VNMYTVEVRS QTSL..AAEV FAIPADIASQ P.LATTLIGE |
| hrv-29 | NNVSSN.LRK VNLYTVQLTS QTTP..AQQV FAIKVDIASQ P.LANTMLGQ |
| dq473512-30 | NNTEDN.ITS EAMYSIKLQS SVDQ...DKI FSLRTDIASQ P.LATTLIGE |
| hrv-30 | NNTEDN.ITS EAMYSIKLQS SVDQ...DKI FSLRTDIASQ P.LATTLIGE |
| hrv-31 | NNTMQN.LRK VNMYTIQLSS QTEM..AQEI FAIKVDIASQ P.LANTMLGE |
| hrv-32 | NNTGNN.TTS INMYTVRLER QTNL..MGEV FAINVDIASH P.LATTLLGE |
| hrv-33 | NSTTAN.NDN TSIYTVTLEK QTNL..AQEV FAIKVDIASN P.LATTLIGE |
| hrv-34 | NNLQQN.ISN TDMYSVVLNN ETEL..AKEI FAIRVDIASQ P.LATTLLGE |
| dq473501-34 | NNLQQN.ISN TDMYSVVLNN ETEL..AKEI FAIRVDIASQ P.LATTLLGE |
| dq473505-36 | NNKEEN.VKS VNMYTVDLRT QTEL..AKEV FSIPVDVASQ P.LATTLIGE |
| hrv-38 | NNVGNN.TKS VDMYTVVLQR QDQL..MQEV FAIRVDVASQ P.LATTLIGE |
| dq473495-38 | NNVGNN.TKS VDMYTVVLQR QDQL..MQEV FAIRVDVASQ P.LATTLIGE |
| ay751783-39 | NNTNER.IGN VNMYTVSLTS QTNT..AEQI FAIKVDIASQ P.LSSTLIGE |
| hrv-40 | NNTMPN.LRN INKYTVQLTS QMDL..AQEI FALKVDVASQ P.LSSTLLGE |
| dq473491-41 | NNTNTN.IRD KSMYTIRLGG ETDI..KEAI FAMRVDIASQ P.LATTLIGE |
| hrv-43 | NNVGTN.VKN VNMYTVLLER QDNL..AQEI FAIRVDIASQ P.LATTLIGE |
| dq473499-44 | NNVNNN.LRK VNLYTVQLTS QTTP..AQQV FAIKVDIASQ P.LANTMLGQ |
| hrv-45 | NNLHIN.KNK IGMYALPLTR QNTP..AAEL FAMPVDITSS P.LATTLLGE |
| dq473506-46 | NNVEQH.KNS VDMYSVVLTK QTNP..AEEI FAIPVDVASQ P.LATTLIGE |
| hrv-47 | NNTAQN.LRK VNMYTIQLSN QTEM..AQEI FAIKVDIASQ P.LASTMLGE |
| dq473496-49 | NNTENN.ITN ENMYSVVLQS TVGE..PDKI FSIKTDVASQ P.LATTLIGE |
| hrv-49-f04 | NNTENN.ITN GDMYSVILQS TIGE..PDKI FSIKTDVASQ P.LATTLIGE |
| hrv-50 | NNLEHN.TTN INMYSVVLSN ETEM..AKEI FAIKVDIASQ P.LATTLIGE |
| hrv-51 | NNTTPV.NNS IGMYTVRVEV QTDF..KKEI FAIPVDVASQ P.LSTTLIGE |
| dq473507-53 | NNTHGV.KNT VNMYTVQVQE QTTP..AKEV FAIPVDVASQ P.LATTLIGE |
| hrv-54 | NNTFAN.LRN VNKYAVSLFS QTDF..AKEI FAIKVDVASQ P.LSSTLLGE |
| hrv-54-f05 | NNTLPN.LRN VSKYAVSLSS QTDF..AKEI FAIKVDVASQ P.LSSTLLGE |
| dq473511-55 | NNVEGN.EKR IDMYTVTLSR ETDV..AKEI LAIRVDIASQ P.LATTMIGE |
| hrv-56 | NNTDAN.LRN INKYTVQLSS QTDL..AKEI FAIKVDVASQ P.LSSTLLGE |
| hrv-57 | NNLEQN.IRK VDMYSVILGQ ETTP..AKEI FAIKVDIASQ P.LATTMIGE |
| hrv-58 | NNVGEN.VKS VNMYTVDLRS QTEL..AQEV FAIPVDIASQ P.LATTLIGE |
| dq473500-59 | NNTASN.IRN VSMYTVQLTS QVNL..AQEI FAIKVDVASQ P.LSSTLLGE |
| hrv-60 | NNVADK.IRS VDMYTVVLQR QDEL..MQEV FAIRVDVASQ P.LATTLIGE |
| hrv-61 | NNTEAN.IRN KNMYTVQLGG EPDP..TTPV FTIRVDIASQ P.LATTLIGE |
| hrv-62 | NNTDNN.LRK VNLYTVELTN QPTP..AQKI FSIKVDIASQ P.LANTMLGQ |
| hrv-63 | NNTNAN.IKN VSMYTVQLTS QTDL..AQEI FAIKVDVASQ P.LSSTLLGE |
| hrv-64 | NNTTAN.TTS VNMYTLQLTK QSPG...HEV FAMKVDIASQ P.LATTMIGE |
| ef173417-64 | NNTTAN.TTS VNMYTLQLTK QSPG...HEV FAMKVDIASQ P.LATTMIGE |
| hrv-65 | NNTIPM.NNK IGMYTVEVGV QTEL..KTEI FAIPVDVASQ P.LATTLIGE |
| hrv-66 | NNTQAN.LKK INIYTIQLSN QPKM..AEEI FAIKVDIASQ P.LSSTLLGQ |
| hrv-67 | NNTGTN.TTS INMYTVQLQR QAEL..MQEI FAINVDIASH P.LATTLLGE |
| hrv-68 | NNTPTA.RNS TNMYTVQVGQ QTKV..AQEV FSILVDVASQ P.LATTLIGE |
| hrv-71 | NNVEAR.KNS ISMYTVEVGV KTTL..AEEV FAIPVDVASQ P.LATTLIGE |
| dq473492-73 | NNIAAN.VTN ESMYAISLES NADI..KQAV FAMRVDITSQ P.LATTLIGE |
| dq473494-74 | NNTAGN.VNN VNMYTVLLER KPDL..MQEV FAIKVDIASQ P.LATTMIGE |
| dq473510-75 | NNTSEN.LKT TNMYTVYLER RSDL..AQEI FAIRVDIASQ P.LATTLIGE |
| dq473502-76 | NSVTGN.TNN TSIYTVTLTR ETSL..AQEI FAIKVDIASD P.LATTLIGE |
| hrv-76 | NSVTGN.TNN TSIYTVTLTR ETSL..AQEI FAIKVDIASD P.LATTLIGE |
| hrv-77 | NNTQEN.LKK VNVYTIQLTN QLKM..AEEI FAIKVDIASQ P.LSNTMLGQ |
| hrv-78 | NNVQAS.KNN VSMYTVMLST QTQT..AQEI FAIPVDVASQ P.LATTLIGE |
| ef173418-78 | NNVQAS.KNN VSMYTVMLST QTQT..AQEI FAIPVDVASQ P.LATTLIGE |
| hrv-80 | NNVQGN.KNS VNMYSIILDK QTTS..AGEV FAIPVDIASQ P.LATTLIGE |
| hrv-81 | NSTRTN.IGN VNMYTVTLQP QTDM..ATEV FAIKVDIASH P.LATTLIGE |
| hrv-81-f06 | NSTRAN.IGN VNMYTVTLQP QTDM..ATEV FAIKVDIASH P.LATTLIGE |
| hrv-81-f07 | NSTRAN.IGN VNMYTVTLQP QTDM..ATEV FAIKVDIASH P.LATTLIGE |
| hrv-82 | NYTSAN.TTS VNMYTVVLDK GDPG...REV FAMRADIATH P.LATTLIGE |
| dq473509-82 | NYTTAN.TTS VNMYTVVLQK ENPG...TEV FAMRVDIATQ P.LATTLIGE |
| hrv-85 | NNTSQN.LRN INKYTVQLTS QVNL..AQEI FAIKVDVASQ P.LSSTLLGE |
| pico-85 | NNTSQN.LRN INKYTVQLTS QVNL..AQEI FAIKVDVASQ P.LSSTLLGE |
| dq473504-88 | NNTEGN.VKS ANMYTVHLAT QDNH..AQEV FSIPVDVASQ P.LATTYLGE |
| a10937-89 | NNTQEN.VRS VNMYTVDLRT QVDL..AKEV FSIPVDIASQ P.LATTLIGE |
| hrv-89 | NNTQEN.VRS VNMYTVDLRT QVDL..AKEV FSIPVDIASQ P.LATTLIGE |
| hrv-89-f08 | NNTQEN.VRS VNMYTVDLRT QVDL..AKEV FSIPVDIASQ P.LATTLIGE |
| hrv-89-f09 | NNTQEN.VRS VNMYTVDLRT QVDL..AKEV FSIPVDIASQ P.LATTLIGE |
| m16248-89 | NNTQEN.VRS VNMYTVDLRT QVDL..AKEV FSIPVDIASQ P.LATTLIGE |
| hrv-90 | DSVEAN.AKN TSMYTVTLSR ETEL..AKEI FAIKVDIASH P.LATTLIGE |
| hrv-94 | NNISSN.TTS VNMYSLVLTK ERPG...HEV FAMKVDIASQ P.LATTIIGE |
| ef173419-94 | NNISSN.TTS VNMYSLVLTK ERPG...HEV FAMKVDIASQ P.LATTIIGE |
| hrv-95 | NNINAS.RNR IDMYALPLTR QPQS..AVKL FTIPIDIASP P.LNTTLLGE |
| hrv-96 | HNTATN.AKN INMYTVQLGG EPNP..KTPV FTIRVDIASQ P.LATTLIGE |
| hrv-98 | NNTIAN.LRN VNKYAVSLSS QTDF..AKEI FAIKVDVASQ P.LSSTLLGE |
| hrv-100 | NNTIAN.LRK VNMYTIQLTN QTEM..AKEI FAIKVDIASQ P.LSSTMIGE |
| ef186077-qpm | NNVNQS..HT LGVYYITISN VSSE...RKI LAIPLDMSNT L.FSTTLMGE |
| ef077279-nat001 | NNIPTM.INS IGAYNIPITG GHDN...KRI VVIPLDTSNT L.FSSTLLGE |
| ny1078 | NNLQSQ.LQD VGVYNITLTG TSNN....RI LAIPLDMSNT L.FSTTLMGE |
| ef582385-c024 | NNIETN.VGQ ISIYNVQVTK KTAD...KPI VVIPMDISNT L.FSTTLLGE |
| ef077280-nat045 | NNIPGQ.VGT VGMYNVTIAS KTAD..QDMI LAIPLQMDNT L.FATTLVGE |
| ef582387-c026 | NNVNQS..QA IGIYYIPISN THNN...RRI LAIPLDMSNT L.FSTTLLGE |
| ef582386-c025 | NNITDQ.KID VPIYSRTVTA RPASNSSDLI LAIPLDMNNT L.FSTTLLGE |
| dg473485-03 | NNTGTN..DN VTNYLIPLHA DRQN...EQI FGTKLYIGDG V.FKTTLLGE |
| ef173422-03 | NNTGTN..DN VTNYLIPLHA DRQN...EQI FGTKLYIGDG V.FKTTLLGE |
| dq473490-04 | NN.AEG.ITG LETYRIPLYQ NRQG...QQV FGFRLYLGDG V.LKTTMLGE |
| hrv-05 | NNTTNT..TG LGMYRIPLVQ NMQG...EQV FGFRLYLGDG V.LKTTLLGE |
| dq473486-06 | NNVHTS..DE VNSYLIPLTA NRQN...DQI FGTKLFIGDG V.FKTTLLGE |
| l05355-14 | NNTHTK..DE VNSYLIPLNA NRQN...EQV FGTNLFIGDG V.FKTTLLGE |
| k02121-14 | NNTHTK..DE VNSYLIPLNA NRQN...EQV FGTNLFIGDG V.FKTTLLGE |
| ef173420-17 | NNKGAT..DS LEEYRIPLTV NKKN...DQI FGTRLFIGDG V.FKTTLFGE |
| hrv-26 | NNAKDV..VG LGMYRIPLVQ NRQE...TQV FGFRLFLGDG V.LKTTLLGE |
| hrv-27 | NNVTDL..DT IEKYRIPLMQ NQQE...QQV FGFSLFLGGG V.LKTTLLGE |
| ef173421-27 | NNVTDL..DT IEKYRIPLMQ NQQE...QQV FGFSLFLGGG V.LKTTLLGE |
| dq473487-35 | NNTHTK..DE VKSYLIPLVA NQQN...QKI FGTDLYIGDG V.FKTTLLGE |
| hrv-35 | NNTHTK..DE VKSYLIPLVA NQQN...QKI FGTDLYIGDG V.FKTTLLGE |
| ef173423-37 | NNLQGS..DE VSNYLIPLTA NKQN...GQI FGTKLFIGDG V.FKTTLLGE |
| hrv-42 | NNTETA..SG LDMYRVPLTQ NKQG...EQV FGFKLYLGDG V.LKTTLIGE |
| dq473488-48 | NNTHTT..DV MDEYRIPLTV GRVN...DQV FGTRMFIGDG V.FKTTLFGE |
| hrv-52 | NNLGTT..DS MSEYRIPLTA GKVN...EQI FGTRLYIGDG V.FKTTLFGE |
| hrv-52-f10 | NNLGVT..DT MSEYRIPLTA GRVN...EQI FGTRLYIGDG V.FKTTLFGE |
| ef173424-52 | NNLGTT..DS MSEYRIPLTA GKVN...EQI FGTRLYIGDG V.FKTTLFGE |
| hrv-69 | NNIGAR..DT MDEYRIPLEV NKVN...EQI FGTRLFIGDG V.FKTTLFGE |
| dq473489-70 | NNKGAT..DT LDEYRIPLTV NKKN...DQI FGTRLFIGDG V.FKTTLFGE |
| hrv-72 | NNTHNK..DA VKSYLIPLQP NRQG...EMV FGTKLFIGDG V.FKTTLLGE |
| hrv-79 | NNIYNK..DK FNSYLIPLTA NQQN...TQI FGTNLFIGDG V.FKTTLLGE |
| hrv-83 | NKTKVN..DE VGNYLIPLIQ GQKN...EQI FGTNLYIGDG V.FKTTLLGE |
| hrv-84 | NNIANN..DN LERYRIPLTP NVQE...TQV FGFSLFLGGT V.LKTTLLGE |
| hrv-86 | NNTTAT..DE VGNYLIPLNV NKQN...EQI FGTNLFIGDG V.FKTTLLGE |
| hrv-91 | NNKGTT..DT LEEYRIPLTV DKKD...GQI FGTRLFIGDG V.FKTTLFGE |
| hrv-92 | NNTKTK..DE VDSYLIPLTQ GLKN...AQI FGTNLYIGDG V.FKTTLLGE |
| ef173425-93 | NNVTEL..DT IDKYRVPLIQ GQQE...QQV FGFSLFLGGK V.LKTTLLGE |
| hrv-97 | NNITLP..DG VEKYRVPLMQ NQQE...SQV FGFSLFLGGD V.LKTTLLGE |
| hrv-99 | NNTKTA..TG LDMYRIPLMQ NKQG...EQV FGFRLYLGDG V.LKTTLLGE |
| | 451 500 |
| annotation | HHHHHBBBBB BBDBBBBBB. ....BBBEBB BBB....... .HHHBHHHHB |
| af499637-cva13 | ILNYYTHWAG SLKFSFLFCG SMMATGKLLV AYSPPGAQPP QDRKAAMLGT |
| af546702-cva21 | ILNYYTHWTG SIRFTFLFCG SMMATGKLLL SYSPPGAKPP TNRKDAMLGT |
| v01149-pv1m | ILNYYTHWAG SLKFTFLFCG FMMATGKLLV SYAPPGADPP KKRKEAMLGT |
| d00239-01 | IASYYTHWTG SLRFSFMFCG TANTTLKLLL AYTPPGIDKP ATRKDAMLGT |
| pico-01 | IASYYTHWTG SLRFSFMFCG TANTTLKLLL AYTPPGIDEP TTRKDAMLGT |
| hrv-01 | IASYYTHWTG SLRFSFMFCG TANTTLKLLL AYTPPGIDEP TTRKDAMLGT |
| x02316-02 | ISSYFTHWTG SLRFSFMFCG TANTTVKLLL AYTPPGIAEP TTRKDAMLGT |
| hrv-07 | MSSYFTHWTG SLRFSFMFCG SACTTLKLLI AYTPPGIAKP TSRKEAMLGT |
| dq473503-07 | MSSCFTHWTG SLRFSFMFCG SACTTLKLLI AYTPPGIAKP TSRKEAMLGT |
| hrv-08 | IASYYTHWTG SIRLSFMFCG SANSSLKLLI AYTPPGAPEP TKRKEAMLGT |
| pico-09 | IASYYTHWTG SLRFSFMFCG SASSTLKLLL AYTPPGIAKP TSRRDAMLGT |
| hrv-09 | IASYYTHWTG SLRFSFMFCG SASSTLKLLL AYTPPGIAKP TSRRDAMLGT |
| hrv-09-f01 | IASYYTHWTG SLRFSFMFCG SASTTLKLLL AYTPPGIAKP ISRRDAMLGT |
| hrv-09-f02 | IASYYTHWTG SLRFSFMFCG SASTTLKLLL AYTPPGIAKP TSRRDAMLGT |
| dq473498-10 | IASYFTHWTG SLRFSFMFCG TANTTLKLLL AYTPPGIKKP TNRKQAMLGT |
| hrv-10 | IASYFTHWTG SLRFSFMFCG TANTTLKLLL AYTPPGIKKP TNRKQAMLGT |
| ef173414-11 | IASYYTHWTG SIRFSFLFCG TANTTLKLLI AYTPPGISKP TSRKEAMLGT |
| ef173415-12 | LSSYFTHWTG SLRFSFMFCG TANTTLKLLI AYTPPGVPKP DTRKKAMLGT |
| hrv-13 | ISSYYTHWTG SLRFSFMFCG SAFTTLKLLI AYTPPGITVP ENRKKAMLGT |
| hrv-13-f03 | ISSYYTHWTG SLRFSFMFCG SAFTTLKLLI AYTPPGITVP ENRKKAMLGT |
| dq473493-15 | IASYYTHWTG SLRFSFMFCG TANTTLKLLL AYTPPGIAKP SNRKEAMLGT |
| 124917-16 | IASYFTHWTG SLRFSFMFCG TANTTLKVLL AYTPPGIGKP RSRKEAMLGT |
| hrv-18 | ISSYYTHWTG SLRFSFLFCG SANTTLKLLV AYTPPGIAKP ADRKAAMLGT |
| hrv-19 | MSSYYTHWTG SLRFSFMFCG TAGTTLKLLL AYTPPGIAKP QKRKDAMLGT |
| hrv-20 | IASYYTHWTG SLRFSFMFCG TANTSLKLLI AYTPPGVPKP ETRQKAMLST |
| hrv-21 | IANYYTHWTG SIRFSFLFCG TANTTLKLLV AYTPPGIKKP ENRKDAMLGT |
| hrv-22 | ISSYFTNWTG SLRFSLMFCG SAGTTLKMLV AYTPPGIAKP ETRKDAMLGT |
| dq473497-23 | ISSYFTHWTG SLRFSFMFCG TANTTVKLLL AYTPPGIAEP TTRKNAMLGT |
| hrv-24 | IASYYTHWTG SLRFSFLFCG TACTTLKLLI AYTPPGIRKP ENRREAMLGT |
| ef173416-24 | IASYYTHWTG SLRFSFLFCG TACTTLKLLI AYTPPGIRKP ENRREAMLGT |
| hrv-25 | IASYFTHWTG SLRFSFMFCG TANTTLKLLL AYTPPGIGEP QDRKQAMLGT |
| dq473508-28 | IASYYTHWTG SLRFSFMFCG TANTSLKLLI AYTPPGVPKP DSRQKAMLST |
| hrv-29 | IASYFTHWTG SLRFSFMFCG TANTTLKLLL AYTPPGIGKP ETRKQAMLGT |
| dq473512-30 | ISNYFTHWTG SLRFSFMFCG TANTTVKLLL AYTPPGIAEP TTRKDAMLGT |
| hrv-30 | ISNYFTHWTG SLRFSFMFCG TANTTVKLLL AYTPPGIAEP TTRKDAMLGT |
| hrv-31 | IASYFTHWTG SLRFSFMFCG TANTTLKLLL AYTPPGIGKP SSRKQAMLGT |
| hrv-32 | IAGYYTHWTG SLRFSFMFCG SASTTLKLLL AYTPPGISKP TTRKEAMLGT |
| hrv-33 | IASYFTHWTG SVRFSFLFCG TANTTLKLLL AYTPPGIRKP ISRKEAMLGT |
| hrv-34 | IASYYTHWTG SLRFSFMFCG SANTTLKLLI AYTPPGISKP TKRKEAMLGT |
| dq473501-34 | IASYYTHWTG SLRFSFMFCG SANTTLKLLI AYTPPGISKP TKRKEAMLGT |
| dq473505-36 | LASYYTHWTG SLRFSFMFCG SASSTLKLLI AYTPPGVGKP KSRREAMLGT |
| hrv-38 | ISSYYTQWTG SLRFSFMFCG TANTTLKLLI AYTPPGIVKP RNRKEAMLGT |
| dq473495-38 | ISSYYTQWTG SLRFSFMFCG TANTTLKLLI AYTPPGIVKP RNRKEAMLGT |
| ay751783-39 | IASYYTHWTG SLRFSFMFCG TANTTLKLLL AYTPPGIDKP TTRKQAMLGT |
| hrv-40 | ICSYYTHWTG SLRFGFMFCG TANTTLKLLI AYTPPGIKKP ENRKQAMLGT |
| dq473491-41 | IASYYTHWTG SLRFSFMFCG SAFTTLKLLI AYTPPGIDVP STRKQAMLGT |
| hrv-43 | IASYYTHWTG SLRFSFMFCG SASTTLKLLL AYTPPGIAKP TTRKDAMLGT |
| dq473499-44 | IASYFTHWTG SLRFSFMFCG TANTTLKLLI AYTPPGIGKP ENRKQAMLGT |
| hrv-45 | IASYYTNWTG SIRLSFMFCG SANTSLKLLL AYTPPGVSKP TSRREAMLGT |
| dq473506-46 | IASYYTHWTG SIRISFMFCG SANTSLKLLI AYTPPGVPKP TSRKEAMLGT |
| hrv-47 | IASYFTHWTG SLRFSFMFCG TANTTLKLLL AYTPPGIGKP RDRKQAMLGT |
| dq473496-49 | ISSYYTHWTG SLRFSFMFCG TANTTVKLLL AYTPPGIAEP TTRKEAMLGT |
| hrv-49-f04 | ISSYYTHWTG SLRFSFMFCG TANTTVKLLL AYTPPGIREP TTRREAMLGT |
| hrv-50 | VSSYYTHWTG SLRFSFLFCG SANTTLKLLI AYTPPGIGKP TNRKEAMLGT |
| hrv-51 | IASYYTHWTG SLRFSFMFCG TASTSLKLLI AYTPPGVDKP NTRTKAMLGT |
| dq473507-53 | IASYYTHWTG SVRFSFMFCG TANTSLKLLI AYTPPGVPKP TSRKDAMLST |
| hrv-54 | ICSYYTHWTG SLRFSFMFCG TANTTLKVLI AYTPPGIKKP ETRKQAMLGT |
| hrv-54-f05 | ICSYYTHWTG SLRFSFMFCG TANTTLKVLI AYTPPGIKKP ETRKQAMLGT |
| dq473511-55 | IASYFTQWTG SLRFSFLFCG TANTTLKILV AYTPPGIRKP QTRREAMLGT |
| hrv-56 | ICSYYTHWTG SLRFSFMFCG TANTTLKVLL AYTPPGIKKP ESRKQAMLGT |
| hrv-57 | IASYYTHWTG SLRFSFLFCG TANTTLKLLL AYTPPGIEKP QSRKDAMLGT |
| hrv-58 | IASYYTHWTG SLRFSFMFCG SASATLKLLI AYTPPGVAKP KSRKEAMLGT |
| dq473500-59 | ICSYYTHWTG SLRFSFMFCG TANTTLKLLL AYTPPGIKKP ENRKQAMLGT |
| hrv-60 | ISSYYTQWTG SLRFSFMFCG TANTTLKLLI AYTPPGIAKP KSRKEAMLGT |
| hrv-61 | VSSYYTHWTG SVRFSFMFCG SALTTLKLLI AYTPPGIRVP RNRKEAMLGT |
| hrv-62 | IASYYTHWTG SLRFSFMFCG TANTTLKLLL AYTPPGIGEP RDRKQAMLGT |
| hrv-63 | ICSYYTHWTG SLRFSFMFCG TANTTLKLLL AYTPPGIKKP ESRKQAMLGT |
| hrv-64 | MSSYFTHWTG SLRFSFMFCG SAGTTLKLLI AYTPPGIEKP TKRKDAMLGT |
| ef173417-64 | MSSYFTHWTG SLRFSFMFCG SAGTTLKLLI AYTPPGIEKP TKRKDAMLGT |
| hrv-65 | IASYYTHWTG SLRFSFMFCG TANTSLKLLI AYTPPGVAKP TTRTMAMLGT |
| hrv-66 | IASYFTHWTG SLRFSFMFCG TANTTLKLLL AYTPPGIRKP ENRKQAMLGT |
| hrv-67 | IASYYTHWTG SLRFSFMFCG SASSTLKLLL AYTPPGISKP TSRKEAMLGT |
| hrv-68 | IASYYTHWTG SLRFSFMFCG TANTSLKLLI AYTPPGVPKP GSRQKAMLST |
| hrv-71 | ISSYYTHWTG SIRFSFMFCG TANTSLKLLI AYTPPGVGKP QNRTQAMLGT |
| dq473492-73 | IASYYTHWTG SLRFSFMFCG SAFSTLKLLL AYTPPGIAVP DSRKKAMLGT |
| dq473494-74 | ISSYFTHWTG SLRFSFMFCG TANSTLKLLL AYTPPGIDKP RSRKDAMLGT |
| dq473510-75 | IASYYTHWTG SLRFSFMFCG SASATLKLLL AYTPPGIAKP TTRKQAMLGT |
| dq473502-76 | IASYYTHWTG SIRFSFLFCG SANTTLKLLI AYTPPGIRKP KNRKEAMLGT |
| hrv-76 | IASYYTHWTG SIRFSFLFCG SANTTLKLLI AYTPPGIRKP KNRKEAMLGT |
| hrv-77 | IASYFTHWTG SLRFSFMFCG TANTTLKLLL AYTPPGIKKP ETRKQAMLGT |
| hrv-78 | IASYYTHWTG SLRFSFMFCG TANTTLKLLI AYTPPGVPKP DTRKKAMLGT |
| ef173418-78 | IASYYTHWTG SLRFSFMFCG TANTTLKLLI AYTPPGVPKP DTRKKAMLGT |
| hrv-80 | IASYYTHWTG SIRFSFMFCG SANTSLKLLI AYTPPGVPKP TSRREAMLGT |
| hrv-81 | IASYFTHWTG SLRFSFMFCG TANTTLKLLL AYTPPGISKP KNRKEAMLGT |
| hrv-81-f06 | IASYFTHWTG SLRFSFMFCG TANTTLKLLL AYTPPGISKP KNRKEAMLGT |
| hrv-81-f07 | IASYFTHWTG SLRFSFMFCG TANTTLKLLL AYTPPGISKP KNRKEAMLGT |
| hrv-82 | IASYYTHWTG SLRFSLMFCG TAGTTLKLLV AYTPPGIAKP TNRKDAMLGT |
| dq473509-82 | IASYYTHWTG SLRFSLMFCG TAGTTLKLLV AYTPPGIAKP ENRKDAMLGT |
| hrv-85 | ICSYYTHWTG SLRFSFMFCG TANTTLKLLL AYTPPGIDKP KNRKQAMLGT |
| pico-85 | ICSYYTHWTG SLRFSFMFCG TANTTLKLLL AYTPPGIDKP KNRKQAMLGT |
| dq473504-88 | MSSYFTHWTG SLRFSFMFCG SASTTLKLLL AYTPPGVGKP TSRREAMLGT |
| a10937-89 | LASYYTHWTG SLRFSFMFCG SASSTLKLLI AYTPPGVGKP KSRREAMLGT |
| hrv-89 | LASYYTHWTG SLRFSFMFCG SASSTLKLLI AYTPPGVGKP KSRREAMLGT |
| hrv-89-f08 | LASYYTHWTG SLRFSFMFCG SASSTLKLLI AYTPPGVGKP RSRREAMLGT |
| hrv-89-f09 | LASYYTHWTG SLRFSFMFCG SASSTLKLLI AYTPPGVGKP RSRREAMLGT |
| m16248-89 | LASYYTHWTG SLRFSFMFCG SASSTLKLLI AYTPPGVGKP KSRREAMLGT |
| hrv-90 | IASYYTHWTG SIRFSFLFCG TANTTLKLLL AYTPPGIRKP RDRREAMLGT |
| hrv-94 | ISSYYTHWTG SLRFSFMFCG SAGTTLKLLI AYTPPGIDKP TKRKDAMLGT |
| ef173419-94 | ISSYYTHWTG SLRFSFMFCG SAGTTLKLLI AYTPPGIDKP TKRKDAMLGT |
| hrv-95 | IASYYTHWTG SIRLSFMFCG SANSSLKLLI AYTPPGAPEP TKRKEAMLGT |
| hrv-96 | IASYYTHWTG SVRFSFMFCG SALTTLKLLI AYTPPGIRVP ENRKEAMLGT |
| hrv-98 | ICSYYTHWTG SLRFSFMFCG TANTTLKVLI AYTPPGIKKP ENRKQAMLGT |
| hrv-100 | IASYFTHWTG SLRFSFMFCG TANTTLKLLL AYTPPGIRKP TTRKQAMLGT |
| ef186077-qpm | VLNYYSNWSG SIKLTFFCVC DSFSTGKFLI AYTPPGADIP ESRRDAMLGT |
| ef077279-nat001 | VLNYFSNWSG SIKLTFFCVC DSFSTGKFLI AYTPPGAGIP ETRRDAMLGV |
| ny1078 | ILNYFSNWSG SIKLTFLCVC DSFSTGKFLI AYTPPGASIP ANRTDAMLGT |
| ef582385-c024 | VLNYFANWSG SISITFMCVC DSFSTGKFLL AYTPPGAALP PDRKTAMLGV |
| ef077280-nat045 | ILNYFSNWSG SIRVTCICVC DSFSTGKFLM AYTPPGGGLP TTRKEAMLGV |
| ef582387-c026 | VLNYYSNWSG SIKLTFFCVC DSFSTGKFLI AYTPPGADIP ETRRDAMLGT |
| ef582386-c025 | VLNYFSTWAG SITLSFLCVC DSFSTGKFLI AYTPPGGGLP KNRKEAMLGV |
| dq473485-03 | IAQYYTHWSG SLRISLMYTG PALSSAKIIL AYTPPGTRGP EDRKEAMLGT |
| ef173422-03 | IAQYYTHWSG SLRISLMYTG PALSSAKIIL AYTPPGTRGP EDRKEAMLGT |
| dq473490-04 | ICQYFTHWAG SLRLSFMYTG PALSSAKLLI AYTPPGTAGP ADRRTAMLGT |
| hrv-05 | LCQYFTHWAG SLRLSFMYTG PALSSAKLLI AYTPPGAQGP TKRKEAMLGT |
| dq473486-06 | IAQYYTHWSG SLRISLMYTG SALSSAKLIL AYTPPGAQGP TNRRDAMLGT |
| 105355-14 | IVQYYTHWSG SLRFSLMYTG PALSSAKLIL AYTPPGARGP QDRREAMLGT |
| k02121-14 | IVQYYTHWSG SLRFSSMYTG PALSSAKLTL AYTPPGARGP QDRREAMLGT |
| ef173420-17 | IAQYYTHWSG SIRISLMYTG PALSSAKLIL AYTPPGTKGP KDRKEAMLGT |
| hrv-26 | ICQYYTHWAG SLRLSFMYTG PALSSAKLLI AYTPPGAEGP TKRKDAMLGT |
| hrv-27 | IAQYYTHWAG SIRISFMYTG PALSSAKLLL AYTPPGAPGP TNRKDAMLGT |
| ef173421-27 | IAQYYTHWAG SIRISFMYTG PALSSAKLLL AYTPPGAPGP TNRKDAMLGT |
| dq473487-35 | IAQYYTHWSG SLKISLMYTG PALSSAKLIL AYTPPGAPGP ETRKDAMLGT |
| hrv-35 | IAQYYTHWSG SLKISLMYTG PALSSAKLIL AYTPPGAPGP ETRKDAMLGT |
| ef173423-37 | IAQYYTHWSG SLRISLMYTG PALSSAKLIL AYTPPGAQGP TNRKDAMLGT |
| hrv-42 | LCQYFTHWAG SLRLSFMYTG PALSSAKLLI AYTPPGAQGP IKRKEAMLGT |
| dq473488-48 | IAQYYTHWSG SIRISLMYTG PALSSAKLIL VYTPPGTEGP KNRKEAMLGT |
| hrv-52 | IAQYFTHWSG SIRISLMYTG PALSSAKLIL AYTPPGSGGP KDRKEAMLGT |
| hrv-52-f10 | IAQYFTHWSG SIRISLMYTG PALSSAKLIL AYTPPGAGGP KDRKEAMLGT |
| ef173424-52 | IAQYFTHWSG SIRISLMYTG PALSSAKLIL AYTPPGSGGP KDRKEAMLGT |
| hrv-69 | IAQYYSHWSG SIRISLMYTG PALSSAKLIL AYTPPGTKGP KTRREAMLGT |
| dq473489-70 | IAQYYTHWSG SIRISLMYTG PALSSAKLIL AYTPPGTEGP KSRKEAMLGT |
| hrv-72 | IVQYYTHWAG SLRFSLMYTG PALSSAKLIL SYTPPGANGP TTRKEAMLGT |
| hrv-79 | IVQYYTHWSG SLRFSLMYTG PALSSAKLIL AYTPPGAQGP TSRKEAMLGT |
| hrv-83 | IVQYYTHWSG SLRISLMYTG PALSSAKLIL AYTPPGAQGP TTKKDAXLGT |
| hrv-84 | IAQYYTHWAG SLRVSFMYTG PALSSAKLLI AYTPPGAPGP KTRKDAMLGT |
| hrv-86 | IAQYYTHWAG SLRFSLMYTG PALSSAKLIL AYTPPGVAGP KNRKEAMLGT |
| hrv-91 | IAQYYTHWSG SIRISLMYTG PALSSAKLIL AYTPPGTEGP KSRREAMLGT |
| hrv-92 | IVQYYAHWSG SLRISLMYTG PALSSAKLTL AYTPPGAQGP TSRKEAMLGT |
| ef173425-93 | IAQYYTHWAG SIRISFMYTG PALSSAKLLL AYTPPGALGP TNRKDAMLGT |
| hrv-97 | IAQYYTHWAG SIRISFMYTG PALSSAKLLV AYTPPGAPGP TTRKEAMLGT |
| hrv-99 | LSQYFTHWAG SLRLSFMYTG PALSSARLLI AYTPPGAQGP TKRKDAMLGT |
| | 501 550 |
| annotation | BFBBBB...B BBBBGBB... ....BBB... HHHH.BBBBH B...BBBB.. |
| af499637-cva13 | HVIWDIGLQS SCTMVVPWIS NTSYRRTVKD DFTEGGYISM FYQTRVVVPA |
| af546702-cva21 | HIIWDLGLQS SCSMVAPWIS NTVYRRCARD DFTEGGFITC FYQTRIVVPA |
| v01149-pv1m | HVIWDIGLQS SCTMVVPWIS NTTYRQTIDD SFTEGGYISV FYQTRIVVPL |
| d00239-01 | HVVWDVGLQS TISLVVPWVS ASHFRLTAND KYSMAGYITC WYQTNLVVPP |
| pico-01 | HVVWDVGLQS TISLVVPWVS ASHFRLTADN KYSMAGYITC WYQTNLVVPP |
| hrv-01 | HVVWDVGLQS TISLVVPWVS ASHFRLTADN KYSMAGYITC WYQTNLVVPP |
| x02316-02 | HVIWDVGLQS TISMVVPWIS ASHYRNTSPG R.STSGYITC WYQTRLVIPP |
| hrv-07 | HLVWDVGLQS TASLVVPWVS ASHFRFTTPD TYSAAGYITC WYQTNFVVPP |
| dq473503-07 | HLVWDVGLQS TASLVVPWVS ASHFRFTTPD TYSAAGYITC WYQTNFVVPP |
| hrv-08 | HIVWDVGLQS TCSLIIPWVS ASHFRNTTPD TYSKAGFVTC WYQTNFVTAP |
| pico-09 | HVVWDVGLQS TISMVVPWVS ASHFRNTTPD KYSLAGFITC WYQTNLVVPA |
| hrv-09 | HVVWDVGLQS TISMVVPWVS ASHFRNTTPD KYSLAGFITC WYQTNLVVPA |
| hrv-09-f01 | HVVWDVGLQS TISMVVPWVS ASHFRNTTPD KYSLAGFITC WYQTNLVVPA |
| hrv-09-f02 | HVVWDVGLQS TISMVVPWVS ASHFRNTTPD KYSLAGFITC WYQTNLVVPA |
| dq473498-10 | HIVWDIGLQS TVSLVVPWVS ASHFRYTTPD TYSAAGYITC WYQTNFVFPP |
| hrv-10 | HIVWDIGLQS TVSLVVPWVS ASHFRYTTPD TYSAAGYITC WYQTNFVFPP |
| ef173414-11 | HVVWDVGLQS TISMVVPWVS ASHYRNTTPD TYSLAGFVTC WYQTNLVVPP |
| ef173415-12 | HLVWDVGLQS TVSMIVPWVS ASHFRFTTPD TYSKAGYITC WYQTNFVVPP |
| hrv-13 | HVIWDVGLQS TVSMVVPWVS ASHYRNTTPD TYSAAGYITC WYQTDLITPP |
| hrv-13-f03 | HVIWDVGLQS TVSMVVPWVS ASHYRNTTPD TYSAAGYITC WYQTDLITPP |
| dq473493-15 | HVVWDVGLQS TISMIVPWVS ASHFRNTTPD TFSSAGYISC WYQTNLVVPP |
| 124917-16 | HVVWDVGLQS TVSLVVPWIS ASQYRFTTPD TYSSAGYITC WYQTNFVVPP |
| hrv-18 | HVVWDVGLQS TISMVVPWVS ASHFRNTTPD TYSLAGYITC WYQTNLIAPT |
| hrv-19 | HVVWDVGLQS TISMIVPWVS ASHYRNTTPD TFSLAGYITC WYQTNLVTPP |
| hrv-20 | HVVWDVGLQS TVSMVVPWVS ASHYRYTTPD TYSQAGYITC WYQTNFIVPP |
| hrv-21 | HVVWDVGLQS TISMVVPWIS ASHYRNTTPD KYSSAGYITC WYQTNLVVPP |
| hrv-22 | HVVWDVGLQS TISMIVPWIS AXHFRNTTPD TFSQAGYITC WYQTSLVTPP |
| dq473497-23 | HIIWDIGLQS TISMVVPWIS ASHYRNTSPD V.STSGFITC WYQTGLVIPP |
| hrv-24 | HVVWDVGLQS TISMVVPWVS ASHFRNTTPD KYSLAGYITC WYQTNLVVPP |
| ef173416-24 | HVVWDVGLQS TISMVVPWVS ASHFRNTTPD KYSLAGYITC WYQTNLVVPP |
| hrv-25 | HIVWDVGLQS TVSLVVPWVS SSHFRYTSRD EHSAAGYITC WYQTSLVFPP |
| dq473508-28 | HVVWDVGLQS TVSLVVPWIS ASHYRYTTPD TYSKAGYITC WYQTNFIVPP |
| hrv-29 | HIVWDVGLQS TVSLVVPWVS ASHFRYTSSD EHSAAGYITC WYQTQLVFPP |
| dq473512-30 | HVIWDIGLQS TISMVVPWIS ASHYRNTSPD V.SPSGYITC WYQTGLVIPP |
| hrv-30 | HVIWDIGLQS TISMVVPWIS ASHYRNTSPD V.SPSGYITC WYQTGLVIPP |
| hrv-31 | HIVWDVGLQS TVSLVVPWVS ASHFRYTSRD EHSAAGYITC WYQTNFVFPP |
| hrv-32 | HVVWDVGLQS TISMIVPWVS ASHFRNTTPD KYSLAGFITC WYQTNLVVPA |
| hrv-33 | HVVWDVGLQS TMSMVVPWVS ASHFRNTTPD TYSLAGYVTC WYQTSLVVPS |
| hrv-34 | HVVWDVGLQS TMSMVVPWVS ASHFRNTTPD KYSLAGYITC WYQTNLIAPQ |
| dq473501-34 | HVVWDVGLQS TMSMVVPWVS ASHFRNTTPD KYSLAGYITC WYQTNLIAPQ |
| dq473505-36 | HLVWDVGLQS TASLVVPWVS ASHFRFTTPD TYSSAGYITC WYQTNFVVPA |
| hrv-38 | HIVWDVGLQS TISMIVPWVS ASHFRNTTPD TYSLAGFITC WYQTNLVVPP |
| dq473495-38 | HIVWDVGLQS TISMIVPWVS ASHFRNTTPD TYSLAGFITC WYQTNLVVPP |
| ay751783-39 | HIVWDVGLQS TVSLVVPWVS ASHFRYTTPD TYSMAGYITC WYQTNFVFPP |
| hrv-40 | HIVWDVGLQS TISLVVPWVS ASHFRYTTPD TYTAAGYITC WYQTNFVFPP |
| dq473491-41 | HVIWDVGLQS TVSMVVPWVS ASHYRNTTPD TYSSAGYITC WYQTNLIIPP |
| hrv-43 | HVVWDVGLQS TISMIVPWIS ASHFRNTTPD AYSLAGYITC WYQTNLVVPP |
| dq473499-44 | HIVWDVGLQS TVSLVVPWVS ASHFRYTSSD ERSAAGYITC WYQTQLVFPP |
| hrv-45 | HLVWDVGLQS TCSLVIPWIS ASHFRNTTPD TYSKAGYVTC WYQTNFITAP |
| dq473506-46 | HLVWDVGLQS TASMVIPWVS ASHYRFTTPD IYTLAGYVTC WYQTNLVVAP |
| hrv-47 | HIVWDVGLQS TVSLVVPWVS ASHFRYTSRD EHSAAGYITC WYQTNFVFPP |
| dq473496-49 | HVIWDVGLQS TISMVVPWIS ASHYRNTSPG T.STSGYITC WYQTRLVIPP |
| hrv-49-f04 | HVIWDIGLQS TISMVVPWIS ASHYRNTSSG A.STAGYVTC WYQTRLVIPP |
| hrv-50 | HVVWDVGLQS TISMVVPWVS ASHYRNTTPD TYSLAGYITC WYQTNLIAPP |
| hrv-51 | HVVWDVGLQS TISMVVPWIS ASHYRFTTPD TYSQAGYITC WYQTNFVTPP |
| dq473507-53 | HVVWDVGLQS TMSMVVPWVS ASHYRYTTPD TYSKAGYITC WYQTNFIVPP |
| hrv-54 | HIVWDVGLQS TVSLVVPWVS ASHFRYTTPD TYSAAGYITC WYQTNFVFPP |
| hrv-54-f05 | HIVWDVGLQS TVSLVVPWVS ASHFRYTTPD TYSAAGYITC WYQTNFVFPP |
| dq473511-55 | HIVWDVGLQS TVSMVVPWVS ASHFRNTTPD TYSLAGYITC WYQTNLVVPP |
| hrv-56 | HIVWDVGLQS TVSLVVPWVS ASHFRYTTPD TYSAAGYITC WYQTNFVFPP |
| hrv-57 | HVVWDVGLQS TISMVVPWVS ASHYRNTTPD TYSQAGYITC WYQTNLIVPP |
| hrv-58 | HLVWDVGLQS TASLVVPWVS ASHFRFTTPD TYSSAGYITC WYQTNFVVPA |
| dq473500-59 | HIVWDVGLQS TISLVVPWVS ASHFRYTTPD NYSSAGFITC WYQTNFVFPP |
| hrv-60 | HIVWDVGLQS TISMIVPWVS ASHFRNTTPD TYSSAGFITC WYQTNLVFPP |
| hrv-61 | HLIWDVGLQS TVSMVVPWVS ASHYRNTTPD TYSIAGFITC WYQTKLVVPP |
| hrv-62 | HIVWDVGLQS TVSLVVPWVS SSHFRYTSRD EHSAAGYITC WYQTSLVFPP |
| hrv-63 | HIVWDVGLQS TISLVVPWVS ASHFRYTTPD NYSAAGYITC WYQTNFVFPP |
| hrv-64 | HVVWDVGLQS TISMIVPWVS ASHYRNTTPD TFSQAGYITX WYQTSLVTPP |
| ef173417-64 | HVVWDVGLQS TISMIVPWVS ASHYRNTTPD TFSQAGYITG WYQTSLVTPP |
| hrv-65 | HLVWDVGLQS TVSMVVPWVS ASHYRFTTPD TYSQAGYITC WYQTNFVVPP |
| hrv-66 | HIVWDVGLQS TVSLVVPWVS ASHFRYTTPD TYSAAGYITC WYQTNFVFPP |
| hrv-67 | HVVWDVGLQS TISMIVPWIS ASHFRNTTPD KYSLAGFITC WYQTNLVVPA |
| hrv-68 | HVVWDVGLQS TVSMVVPWVS ASHYRYTTPD TYSQAGYITC WYQTNFIVPP |
| hrv-71 | HMIWDVGLQS TVSMVVPWIS ASNYRFTTPD AYSQAGFITC WYQTNFVVPP |
| dq473492-73 | HVIWDVGLQS TVSIVVPWVS ASHYRNTTPD TYSLAGYITC WYQTKLVEPP |
| dq473494-74 | HVVWDVGLQS TISMIVPWVS ASHFRNTTPD TFSSAGFITC WYQTNLVVPP |
| dq473510-75 | HVVWDVGLQS TISMIVPWVS ASHFRNTTPD TYSLAGYVTC WYQTNLVVPP |
| dq473502-76 | HVVWDVGLQS TISMVVPWVS ASHFRNTTPD TYSLAGYVTC WYQTNLVVPP |
| hrv-76 | HVVWDVGLQS TISMVVPWVS ASHFRNTTPD TYSLAGYVTC WYQTNLVVPP |
| hrv-77 | HIVWDVGLQS TVSLVVPWVS ASHFRYTTPD TYSAAGYITC WYQTNFVFPP |
| hrv-78 | HLVWDVGLQS TVSLVVPWVS ASHFRFTTPD TYSKAGYITC WYQTNFVVPP |
| ef173418-78 | HLVWDVGLQS TVSLVVPWVS ASHFRFTTPD TYSKAGYITC WYQTNFVVPP |
| hrv-80 | HLVWDVGLQS TASMVVPWVS ASHYRFTTPD MYSLAGYITC WYQTNLIVAP |
| hrv-81 | HVVWDVGLQS TVSLVVPWVS ASQYRFTTPD TYSXAGYITC WYQTNFVVPP |
| hrv-81-f06 | HVVWDVGLQS TVSLVVPWVS ASQYRFTTPD TYSSAGYITC WYQTNFVVPP |
| hrv-81-f07 | HVVWDVGLQS TVSLVVPWVS ASQYRFTTPD TYSSAGYITC WYQTNFVVPP |
| hrv-82 | HVVWDVGLQS TISMIVPWVS ASHFRNTTPD KFSQAGYITC WYQTALVTPP |
| dq473509-82 | HVVWDVGLQS TISMIVPWVS ASHFRNTTPD KFSQAGYITC WYQTTLVTPP |
| hrv-85 | HIVWDVGLQS TISLVVPWVS ASHFRYTTPD TYSAAGYITC WYQTNFVFPP |
| pico-85 | HIVWDVGLQS TISLVVPWVS ASHFRYTTPD TYSRAGYITC WYQTNFVFPP |
| dq473504-88 | HLVWDVGLQS TASLVVPWVS ASHFRFTTPD TYSSAGYITC WYQTNFVVPP |
| a10937-89 | HLVWDVGLQS TASLVVPWVS ASHFRFTTPD TYSSAGYITC WYQTNFVVPD |
| hrv-89 | HLVWDVGLQS TASLVVPWVS ASHFRFTTPD TYSSAGYITC WYQTNFVVPD |
| hrv-89-f08 | HLVWDVGLQS TASLVVPWVS ASHFRFTTPD TYSSAGYITC WYQTNFVVPD |
| hrv-89-f09 | HLVWDVGLQS TASLVVPWVS ASHFRFTTPD TYSSAGYITC WYQTNFVVPD |
| m16248-89 | HLVWDVGLQS TASLVVPWVS ASHFRFTTPD TYSSAGYITC WYQTNFVVPD |
| hrv-90 | HVVWDVGLQS TISMVVPWVS ASHYRNTTPD KYSLAGFVTC WYQTNFIVPP |
| hrv-94 | HVVWDVGLQS TISMIVPWVS ASHYRNTTPD KFSQAGYITC WYQTSLVSPP |
| ef173419-94 | HVVWDVGLQS TISMIVPWVS ASHYRNTTPD KFSQAGYITC WYQTSLVSPP |
| hrv-95 | HIVWDVGLQS TCSLIIPWVS ASHFRNTTPD TYSKAGFVTC WYQTNFVTAP |
| hrv-96 | HLIWDVGLQS TVSMVVPWIS ASHFRNTTPD TYSLAGYITC WYQTKLVAPP |
| hrv-98 | HIVWDVGLQS TVSLVVPWVS ASHFRYTTPD TYSAAGYITC WYQTNLVFPP |
| hrv-100 | HIVWDVGLQS TVSLVVPWVS ASHFRYTTPD TYSAAGYITC WYQTNFVFPP |
| ef186077-qpm | HVVWDLGLQS SCHLVVPWIS SHFFRRTRKD NYTESGFVTL WHQTAFVSNG |
| ef077279-nat001 | HVVWDLGLQS SCHLVVPWIS SHFFRRTKKD DYTEGGFITL WYQTAFVSN. |
| ny1078 | HIIWDLGLQS SCHMVIPWIS SHFFRRTRKD NFTEGGFVTL WHQTAFVSNG |
| ef582385-c024 | HVIWDLGLQS SCTIVVPWMS GTFHRRTKAD NYTQAGYVSL WYQTDFVSPN |
| ef077280-nat045 | HVVWDLGLQS SCTLVAPWMS STFYRRTKGS NYTSGGYITL WYQTNFVATT |
| ef582387-c026 | HVVWDLGLQS SCHLVVPWIS SHFFRRTRKD NYTESGFVTL WHQTAFVSNG |
| ef582386-c025 | HVIWDLGLQS TCTIVVPWIA QGYYRHTKAD NYTESGYVTL WYQTNFV.PG |
| dq473485-03 | HVVWDIGLQS TIVMTIPWTS GVQFRYTDPD TYTSAGYLSC WYQTSLILPP |
| ef173422-03 | HVVWDIGLQS TIVMTIPWTS GVQFRYTDPD TYTSAGYLSC WYQTSLILPP |
| dq473490-04 | HVVWDIGLQS TVVLNIPWTS GVQFRYTDPD TYTSAGFVSC WYQTSLVLPP |
| hrv-05 | HVVWDIGLQS TVVLNIPWTS GVQYRYTDPD TYTSAGFVSC WYQTSLVLPP |
| dq473486-06 | HVVWDIGLQS TIVMTIPWTS GVQFRYTDPD TYTSAGYLSC WYQTSLILPP |
| 105355-14 | HVVWDIGLQS TIVMTIPWTS GVQFRYTDPD TYTSAGFLSC WYQTSLILPP |
| k02121-14 | HVVWDIGLQS TIVMTIPWTS GVQFRYTDPD TYTSAGFLSC WYQTSLILPP |
| ef173420-17 | HVVWDVGLQS TVVMNIPWTS GVQFRYTDPD TYTSAGYLSC WYQTSLILPP |
| hrv-26 | HVVWDIGLQS TVVLNIPWTS GVQYRYTDPD TYTSAGYVSC WYQTSLVLPP |
| hrv-27 | HIIWDIGLQS TIVMNIPWTA GSQFRYTDPD TYTHPGFISC WYQTSLVLPP |
| ef173421-27 | HIIWDIGLQS TIVMNIPWTA GSQFRYTDPD TYTHPGFISC WYQTSLVLPP |
| dq473487-35 | HVVWDIGLQS TIVMTIPWTS GVQFRYTDKD TYTSAGFLSC WYQTSLILPP |
| hrv-35 | HVVWDIGLQS TIVMTIPWTS GVQFRYTDKD TYTSAGFLSC WYQTSLILPP |
| ef173423-37 | HVVWDIGLQS TIVMTIPWTS GVQFRYTDPD TYTSAGYLSC WYQTSLILPP |
| hrv-42 | HVIWDIGLQS TVVLNIPWTS GVQFRYTDPD TYTSAGFISC WYQTSLVLPP |
| dq473488-48 | HVVWDVGLQS TVVMNIPWTS GVQFRYTDPD TYTSAGFLSC WYQTSLILPP |
| hrv-52 | HVVWDVGLQS TVVMNIPWTS GVQFRFTDPD TYTSAGFLSC WYQTSLILPP |
| hrv-52-f10 | HVVWDVGLQS TVVMNIPWTS GVQFRYTDPD TYTSAGFLSC WYQTSLILPP |
| ef173424-52 | HVVWDVGLQS TVVMNIPWTS GVQFRFTDPD TYTSAGFLSC WYQTSLILPP |
| hrv-69 | HVVWDVGLQS TVVMNIPWTS GVQFRYTDPD TYTSAGHLSC WYQTSLILPP |
| dq473489-70 | HVVWDVGLQS TVVMNIPWTS GVQFRYTDPD TYTSAGHLSC WYQTSLILPP |
| hrv-72 | HVVWDIGLQS TIVMTIPWTS GVQFRYTDPD TYTSAGFLSC WYQTSLILPP |
| hrv-79 | HVVWDIGLQS TIVMTIPWTS GVQFRYTDKD TYTSAGFLSC WYQTSLILPP |
| hrv-83 | HVVWDIGLQS TIVMTIPWTS GVQFRYTDKD TYTSAGFLSC WYQTSLILPP |
| hrv-84 | HLVWDIGLQS TVVMNIPWTS GSQFRYTDPD TYTHPGYISC WYQTSLILPP |
| hrv-86 | HVVWDIGLQS TIIMNIPWTS GVQFRYTDPD TYTSAGHLSC WYQTSLILPP |
| hrv-91 | HVVWDVGLQS TVVMNIPWTS GVQFRYTDPD TYTSAGHLSC WYQTSLILPP |
| hrv-92 | HVVWDIGLQS TIVMTIPWTS GVQFRYTDKD TYTSAGFLSC WYQTSLILPP |
| ef173425-93 | HVIWDIGLQS TVVMNIPWTA GAQFRYTDPD TYTHPGFISC WYQTSLVLPP |
| hrv-97 | HVVWDIGLQS TVVMNIPWTA GAQFRYTDPD TYTHPGYVSC WYQTSLVLPP |
| hrv-99 | HVVWDIGLQS TVVLNIPWTS GVQFRYTDPD TYTSAGFVSC WYQTSLVLPP |
| | 551 600 |
| annotation | .....BBBBB IBBBB...BB BBB....... ......QNP. .......... |
| af499637-cva13 | STPTSMDILC FISACNDFTV RLLRDTTHIT QS.ALPQGLE DLIQQVASNA |
| af546702-cva21 | STPTSMFMLG FVSACPDFSV RLLKDTPHIS QSKLIGRTQG IEDLIDTAIK |
| v01149-pv1m | STPREMDILG FVSACNDFSV RLLRDTTHIE QK.ALAQGLG QMLESMIDNT |
| d00239-01 | NTPQTADMLC FVSACKDFCL RMARDTDLHI QSGPIEQNPV ENYIDEVLNE |
| pico-01 | STPQTADMLC FVSACKDFCL RMARDTDLHI QSGPIEQNPV ENYIDEVLNE |
| hrv-01 | STPQTADMLC FVSACKDFCL RMARDTDLHI QSGPIEQNPV ENYIDEVLNE |
| x02316-02 | QTPPTARLLC FVSGCKDFCL RMARDTNLHL QSGAIAQNPV ENYIDEVLNE |
| hrv-07 | NTPRSAKMVC MVSACKDFCL RLARDTNLHM QEGVLEQNPV ENYVDSLLNE |
| dq473503-07 | NTPRSAKMVC MVSACKDFCL RLARDTNLHM QEGVLEQNPV ENYVDSLLNE |
| hrv-08 | DAPTTADIIC LVSACKDFCL RMARDTDQHT QVGAITQNPI EQFTEAVLNE |
| pico-09 | DTPTTANLLC FVSACKDFCL RMARDTNLHK QSGPITQNPV ENYIDQVLNE |
| hrv-09 | DTPTTANLLC FVSACKDFCL RMARDTNLHK QSGPITQNPV ENYIDQVLNE |
| hrv-09-f01 | DTPTTANLLC FVSACKDFCL RMARDTNLHK QSGPITQNPV ENYIDQVLNE |
| hrv-09-f02 | DTPTTANLLC FVSACKDFCL RMARDTNLHK QSGPITQNPV ENYIDQVLNE |
| dq473498-10 | NTPSTADMLC FVSACKDFCL RMARDTNMHI QSDAITQNPV ENYIDNVLNE |
| hrv-10 | NTPSTADMLC FVSACKDFCL RMARDTNMHI QSDAITQNPV ENYIDNVLNE |
| ef173414-11 | STPTTAKMLC FVSGCKDFCL RMARDTNLHL QSGPIEQNPV EDYVDGILNE |
| ef173415-12 | GTPNSANMLC FVSACKDFCL RMARDTDLHS QDTPITQNPV ERYVDEVLNE |
| hrv-13 | NTAPVADMIC FVSGCKDFCL RMARDTNLHK QSGVIAQNPV ERYVDEVLNE |
| hrv-13-f03 | NTAPVADMIC FVSGCKDFCL RMARDTNLHK QSGVIAQNPV ERYVDEVLNE |
| dq473493-15 | NTPTTAKMLC FVSGCKDFCL RMARDTDLHK QRGNITQNPV ENYIDEVLNE |
| 124917-16 | NTPNTAEMLC FVSGCNHFCL RMARDTDLHK QTGPITQNPV ERYVDEVLNE |
| hrv-18 | NAPQTARMLC FVSACKDFCL RMARDTNLHT QSGTIEQNPV ENYIDEVLNE |
| hrv-19 | NTPSTARMLC FVSGCKDFCL RMARDTNLHK QTGTITQNPV EKYVDTILNE |
| hrv-20 | NTPREADIIC FVSGCKDFCL RMARDTNLHT QVGVMEQNPV ERYTEAILNE |
| hrv-21 | NTPTSAKMLC FVSGCKDFCL RMARDTSLHK QSAPITQNPV ENYIDEVLNE |
| hrv-22 | STPETARMLC FVSGCKDFCL RMARDTNLHK QIGAITQNPV EKYIDGVLNE |
| dq473497-23 | DTPSTARLLC FVSGCKDFCL RMARDTNLHM QKGIIAQNPI ENYVDEVLNE |
| hrv-24 | NTPTTAKMLC FVSGCKDFCL RMARDTNLHL QSAPITQNPV ENYIDEVLNE |
| ef173416-24 | NTPTTAKMLC FVSGCKDFCL RMARDTNLHL QSAPITQNPV ENYIDEVLNE |
| hrv-25 | NTPSTADMLC FVSACKDFCL RMARDTDMHV QSEAIEQNPI ENYVDQVLNE |
| dq473508-28 | STPNSANIIC FVSACKDFCL RMARDTNLHT QVGAITQNPV EKYTEALLNE |
| hrv-29 | NTPNTADMLC FVSACKDFCL RMARDTDMHI HNEIITQNPV ENYVDEVLNE |
| dq473512-30 | ETPPTARLLC FVSGCKDFCL RMARDTSLHM QKGVIEQNPV ENFVDEVLSE |
| hrv-30 | ETPPTARLLC FVSGCKDFCL RMARDTSLHM QKGVIEQNPV ENFVDEVLSE |
| hrv-31 | NTPNTANMLC FVSACKDFCL RMARDTDMHI QDDAITQNPV ENYVEEVLNE |
| hrv-32 | DTPATANLLC FVSACKDFCL RMARDTNLHK QSGPITQNPV ENYIDQVLNE |
| hrv-33 | STPITAKMLC FVSGCKDFCL RMARDTNLHL QSGIITQNPV ENYIEEVLNE |
| hrv-34 | NTPQTAKILC FVSACKDFCL RMARDTNLHK QSGPIEQNPV ENYIDEVLNE |
| dq473501-34 | NTPQTAKILC FVSACKDFCL RMARDTNLHK QSGPIEQNPV ENYIDEVLNE |
| dq473505-36 | STPDNAKMVC MVSACKDFCL RLARDTNLHM QEGVLTQNPV ENYIDNVLNE |
| hrv-38 | DTPATARMLC FVSGCKDFCL RMARDTNLHK QSGAITQNPV EEYIDGVLNE |
| dq473495-38 | DTPATARMLC FVSGCKDFCL RMARDTNLHK QSGAITQNPV EEYIDGVLNE |
| ay751783-39 | NTPNNANMIC FVSGCKDFCL RMARDTDMHV QNVPITQNPV ENYIDEVLNE |
| hrv-40 | NTPATADMLC FVSACKDFCL RMARDTDMHV QNVPITQNPV ERYVDEVLNE |
| dq473491-41 | NAAPVADMIC FVSGCKDFCL RMARDTNLHK QSGVIEQNPV ERYVDEVLNE |
| hrv-43 | NTPSTAKMLC FVSGCKDFCL RMARDTNLHK QSGAIVQNPV ENYVDEILNQ |
| dq473499-44 | NTPNTADMLC FVSACKDFCL RMARDTDMHT HNEIITQNPV ENYVDEVLNE |
| hrv-45 | NTPPTADIIC LVSACKDFCL RMARDTNQHT QLGAIEQNPV EQFAEAVLDQ |
| dq473506-46 | NTPTTARMIC FVSGCKDFCL RMARDTNLHS QTGAITQNPV EKYTEAVLNE |
| hrv-47 | NTPNRADMLC FVSACKDFCL RMARDTDMHI QNDAITQNPV ENYVEEVLNE |
| dq473496-49 | QTPATARLLC FVSGCKDFCL RMARDTNLHL QSGVIEQNPV EHYIDEVLNE |
| hrv-49-f04 | QTPPTARLLC FVSGCKDFCL RMARDTNLHL QSGPIEQNPV EQYIDEVLNE |
| hrv-50 | NTPSTARMLC FVSGCKDFCL RMARDTNLHT QSGNIEQNPV ENYIDEVLNE |
| hrv-51 | STPKYADIIC FVSGCKDFCL RMARDTNLHS QTVNITQNPV EKYTEALLNE |
| dq473507-53 | STPNSADIIC FVSGCKDFCL RMARDTNLHT QVGAITQNPV EKYTEAILNE |
| hrv-54 | NTPNSADMLC FVSACKDFCL RMARDTDMHV QNVPIEQNPV ERYVDEVLNE |
| hrv-54-f05 | NTPNNADMLC FVSACKDFCL RMARDTDMHV QNVPIEQNPV EKYVDEVLNE |
| dq473511-55 | NTPTTARMLC FVSGCKDFCL RMARDTNLHL QSAPITQNPV ERYVDEVLNE |
| hrv-56 | NTPNTADMLC FVSACKDFCL RMARDTDMHV QNVPITQNPV ERYVDDVLNE |
| hrv-57 | NTPTTARMLC FVSGCKDFCL RMARDTNLHM QSMPITQNPV ENFVDEVLNE |
| hrv-58 | NTPANAKMAC MVSACKDFCL RLARDTNLHM QDGVLTQNPV ENYIDNVLNE |
| dq473500-59 | NTPSSADMLC FVSACKDFCL RMARDTDTHT QIVPITQNPV ENYVNDVLNE |
| hrv-60 | DTPTTARMLC FVSGCKDFCL RMARDTNLHK QSGIITQNPV ESYIDGVLNE |
| hrv-61 | NTASTADMLC FVSGCKDFCL RMARDTNLHK QSGPIEQNPV ERYVDEVLNE |
| hrv-62 | NTPGTADMLC FVSACKDFCL RMARDTDMHT QSDAIEQNPI ENYVDQVLNE |
| hrv-63 | NTPNSADMLC FVSACKDFCL RMARDTDMHV QNVPITQNPV ENYVNDVLNE |
| hrv-64 | STPQTARMLC FVSGCKDFCL RMARDTNLHK QIGAITQNPV EQYIDGVLNE |
| ef173417-64 | STPQTARMLC FVSGCKDFCL RMARDTNLHK QIGAITQNPV EQYIDGVLNE |
| hrv-65 | NTPTTANIIC FVSGCKDFCL RMARDTNLHS QTVNITQNPI EKYTEALLNE |
| hrv-66 | NTPSTADMLC FVSACKDFCL RMARDTDMHV QNDAITQNPV EDYVEGVLNE |
| hrv-67 | NTPTTANLLC FVSACKDFCL RMARDTNLHK QTGPITQNPV EDYVDQVLNE |
| hrv-68 | NTPNTADIIC FVSGCKDFCL RMARDTNLHT QAGVIEQNPV EKYTEAVLNE |
| hrv-71 | NTPKTANIIC FVSGCKDFCL RMARDTNLHS QTVNIEQNPV EKYTEAILNE |
| dq473492-73 | NTTPVADMIC FVSGCKDFCL RMARDTNLHK QSGVIAQNPV ERYVDEVLNE |
| dq473494-74 | NTPVTARMLC FVSGCKDFCL RMARDTDLHK QRGNITQNPV ENYIDEVLNE |
| dq473510-75 | NTPPNAKMLC FVSGCKDFCL RMARDTNLHK QSGVIEQNPV ENYVDEILNQ |
| dq473502-76 | NTPTTAKMLC FVSGCKDFCL RMARDTNLHL QSGPITQNPV ENYVDEILNE |
| hrv-76 | NTPTTAKMLC FVSGCKDFCL RMARDTNLHL QSGPITQNPV ENYVDEILNE |
| hrv-77 | NTPNTADMLC FVSACKDFCL RMARDTDMHI QNDAITQNPV EDYIDNVLNE |
| hrv-78 | GTPNTANMLC FVSGCKDFCL RMARDTDLHV QTTPITQNPV EEYVDQVLNE |
| ef173418-78 | GTPNTANMLC FVSGCKDFCL RMARDTDLHV QTTPITQNPV EEYVDQVLNE |
| hrv-80 | NTPSTARMIC FVSGCKDFCL RMARDTNLHV QTGPITQNPV EKYTEAILNE |
| hrv-81 | NTPNTAEMLC FVSGCKDFCL RMARDTDLHK QSGPITQNPV ERYVDEVLNE |
| hrv-81-f06 | NTPNTAEMLC FVSGCKDFCL RMARDTDLHK QSGPIEQNPV ERYVDEVLNE |
| hrv-81-f07 | NTPNTAEMLC FVSGCKDFCL RMARDTDLHK QSGPIEQNPV ERYVDEVLNE |
| hrv-82 | STPATARMLC FVSGCKDFCL RMARDTNLHK QIGPITQNPV EKYVDSVLNE |
| dq473509-82 | STPATARMLC FVSGCKDFCL RMARDTNLHK QVGPITQNPV EKYVDSVLNE |
| hrv-85 | NTPATADMLC FVSACKDFCL RMARDTDMHV QDVPITQNPV EKYVDEVLNE |
| pico-85 | NTPATADMLC FVSACKDFCL RMARDTDMHV QDVPITQNPV EKYVDEVLNE |
| dq473504-88 | GTPKNAKMVC MVSACKDFCL RLARDTNLHM QEGVLTQNPV ENYVDNLLNE |
| a10937-89 | STPDNAKMVC MVSACKDFCL RLARDTNLHT QEGVLTQNPV ENYIDSVLNE |
| hrv-89 | STPDNAKMVC MVSACKDFCL RLARDTNLHT QEGVLTQNPV ENYIDSVLNE |
| hrv-89-f08 | STPDNAKMVC MVSACKDFCL RLARDTNLHT QEGVLTQNPV ENYIDSVLNE |
| hrv-89-f09 | STPDNAKMVC MVSACKDFCL RLARDTNLHT QEGVLTQNPV ENYIDSVLNE |
| m16248-89 | STPDNAKMVC MVSACKDFCL RLARDTNLHT QEGVLTQNPV ENYIDSVLNE |
| hrv-90 | NTPTTAKMLC FVSGCKDFCL RMARDTNLHL QSGPITQNPV ENYIDEVLNE |
| hrv-94 | STPQQARMLC FVSGCKDFCL RMARDTNLHK QIGAITQNPV EKYIDGVLNE |
| ef173419-94 | STPQQARMLC FVSGCKDFCL RMARDTNLHK QIGAITQNPV EKYIDGVLNE |
| hrv-95 | DAPTTADIIC LVSACKDFCL RMARDTDQHT QVGAITQNPI EQFTEAVLNE |
| hrv-96 | NTPTTADMLC FVSGCKDFCL RMARDTNLHK QSGPIAQNPV ERYVDEVLNE |
| hrv-98 | NTPNSADMLC FVSACKDFCL RMARDTDMHV QEVPIEQNPV ERYVDEVLNE |
| hrv-100 | NAPNTADMLC FVSACKDFCL RMARDTDMHI QNDAIQQNPV ENYVEGVLNE |
| ef186077-qpm | ...TSGSIMV TCSACSDMSV RMLRDTPMMK QEDNITQNPV EEFVEHTLKE |
| ef077279-nat001 | ..GATGAIMV TCSACSDMSV RMLRDTPMMQ QDENIAQNPV EEFVDHTLQK |
| ny1078 | ...QVGSIMV TCSACSDMSV RMLRDTPMMK QEINIAQNPV EKFIDQTLEE |
| ef582385-c024 | ..DGIGTIVA TCSGCPDLSV RMLRDTPMMK QEKDLTQNPV EDYIDKVVDT |
| ef077280-nat045 | T.GGTGTIIA TCSACPDLSV RMMRDTPMIK QPENNIQNPV DNFVDEVLKE |
| ef582387-c026 | ...TTGSIMV TCSACSDMSV RMLRDTPMMR QDEDITQNPV EEFVEHTLKE |
| ef582386-c025 | SAIAQGSILV TCSACPDMAV RMLRDTPMIT QDKNTLQNPV EQFVDNVLEE |
| dq473485-03 | QTSGQVYLLS FISACPDFKL RLMKDTQTIS QTDALTEGLS DELEEVIVEK |
| ef173422-03 | QTSGQVYLLS FISACPDFKL RLMKDTQTIS QTDALTEGLS DELEEVIVEK |
| dq473490-04 | QVEQTVYMLG FVSACPDFKL RLMKDTQSIQ QETALTEGLG EDLMEVIVDK |
| hrv-05 | QTQQTVYMLG FISACPDFKL RLMKDTQSIH QETALTEGLE DDLVEVIVDK |
| dq473486-06 | NTTGQVHLLS FISACPDFKL RLMKDTQTIS QTDALTEGLG DELEEVIVEK |
| 105355-14 | ETTGQVYLLS FISACPDFKL RLMKDTQTIS QTVALTEGLG DELEEVIVEK |
| k02121-14 | ETTGQVYLLS FISACPDFKL RLMKDTQTIS QTVALTEGLG DELEEVIVEK |
| ef173420-17 | DTTGTVYLLA FISACPDFKL RLMKDTQTIS QTEALMEGFE DELEEVVIDK |
| hrv-26 | QSQQTVCLLG FISACPDFKL RLMKDTQSIY QDSALTEGLE DDLVEVIVDK |
| hrv-27 | DTNQKVFIIG FVSACPDFKL RLLKDTSSIS QTEALTEGLG EELEEVIVDK |
| ef173421-27 | DTNQKVFIIG FVSACPDFKL RLLKDTSSIS QTEALTEGLG EELEEVIVDK |
| dq473487-35 | QTQGQVYLLS FISACSDFKL RLMKDTQTIS QTDALPEGLG EELEEVIVEK |
| hrv-35 | QTQGQVYLLS FISACSDFKL RLMKDTQTIS QTDALPEGLG EELEEVIVEK |
| ef173423-37 | NTAGQVYLLS FISACSDFKL RLMKDTQTIS QTDALTEGLG DELEEVIVEK |
| hrv-42 | QTQQTVYILG FISACPDFKL RLMKDTQSIH QETALTEGLE DELMEVIVDK |
| dq473488-48 | DTSGAVYLLA FVSACPDFKL RLMKDTQTIS QTEALTEGFE EELEEVVVDK |
| hrv-52 | ETSGTVYLLA FVSACPDFKL RLMKDTQTIS QTEALTEGFG EELEEVVVDK |
| hrv-52-f10 | ETSGTVYLLA FVSACPDFKL RLMKDTQTIS QTEALTEGLG EELEEVVVDK |
| ef173424-52 | ETSGTVYLLA FVSACPDFKL RLMKDTQTIS QTEALTEGFG EELEEVVVDK |
| hrv-69 | DTAGTVYLLA FISACPDFKL RLMKDTQTIS QTEALTEGLG EELEEVVIDK |
| dq473489-70 | DTTGTVYLLA FISACPDFKL RLMKDTQTIS QTEALMEGFE GELEEVVIDK |
| hrv-72 | DTPTEVYMLS FISACPDFKL RLMKDTQTIS QTVALTEGLN DELEEVIVEK |
| hrv-79 | QTAGKVYLLS FISACPDFKL RLMKDTQTIS QTTALTEGLN DELEEVIVEK |
| hrv-83 | ETSGTVYLLS FVSACPDFKL RLMKDTQTIS QTSALTEGLN DELEEVIVEK |
| hrv-84 | ETNQTVYLLG FISACPDFKL RLMKDTSSIS QERALTEGLE DVLEEVIVDK |
| hrv-86 | QTSGTAYLLS FISACPDFKL RLMKDTQTIS QTDALEEGLG DELEEVIVEK |
| hrv-91 | DTTGAVHLLA FISACPDFKL RLXKDTQTIS QTEALTEGFE GELEEVVIDK |
| hrv-92 | QTAGTVYLLS FISACPDFKL RLMKDTQTIS QTTALTEGLN DELEEVIVEK |
| ef173425-93 | GTNQNVYILG FVSACPDFKL RLLKDTSSIS QTEALTEGLG QELEEVIVDK |
| hrv-97 | ETNQSVYILG FISACPDFKL RLMKDTSSIS QSEALTEGLG EELEEVIVDK |
| hrv-99 | QTQQTVYILG FVSACPDFKL RLMKDTQSIY QDTALTEGLE EDLVEVIVDK |
| | 601 650 |
| annotation | .......... .......... .......... ......HHHH H.....HHHH |
| af499637-cva13 | LQLSQPTRPA LPPAEQSVPN TNQTTPEHSK EVPALTAVET GATNPLEPGD |
| af546702-cva21 | NALRVSQ... .PPSTQS... TEATSGVNSQ EVPALTAVET GASGQAIPSD |
| v01149-pv1m | VRETVGA... .ATSRDALPN TEASGPTHSK EIPALTAVET GATNPLVPSD |
| d00239-01 | VLV....... .......VPN IKESHHTTSN SAPLLDAAET GHTSNVQPED |
| pico-01 | VLV....... .......VPN IKESHHTTSN SAPLLDAAET GHTSNVQPED |
| hrv-01 | VLV....... .......VPN IKESHHTTSN SAPLLDAAET GHTSNVQPED |
| x02316-02 | VLV....... .......VPN INSSNPTTSN SAPALDAAET GHTSSVQPED |
| hrv-07 | VLV....... .......VPN IQPSTSVSSH SVPALDAAET GHTSSVQPED |
| dq473503-07 | VLV....... .......VPN IQPSTSVSSH SVPALDAAET GHTSSVQPED |
| hrv-08 | VLV....... .......VPN TQASNGSIAN SAPALDAAET GHTSSVQPED |
| pico-09 | VLV....... .......VPN IKESNPTTSN SAPALDAAET GHTSNVQPED |
| hrv-09 | VLV....... .......VPN IKESNPTTSN SAPALDAAET GHTSNVQPED |
| hrv-09-f01 | VLV....... .......VPN IKESNPTTSN SAPALDAAET GHTSNVQPED |
| hrv-09-f02 | VLV....... .......VPN IKESNPTTSN SAPALDAAET GHTSNVQPED |
| dq473498-10 | VLV....... .......VPN IRESHPSTSN SAPILDAAET GHTSSVQPED |
| hrv-10 | VLV....... .......VPN IRESHPSTSN SAPILDAAET GHTSSVQPED |
| ef173414-11 | VLV....... .......VPN IKESQATTSN SAPALDAAET GHTSKVQPED |
| ef173415-12 | VLV....... .......VPN INKSNGQLSN AAPALDAAET GHTSQTQPED |
| hrv-13 | VLV....... .......VPN ISESSSTTSN SAPALDAAET GHTSSVQPED |
| hrv-13-f03 | VLV....... .......VPN ISESKPTTSN SAPALDAAET GHTSSVQPED |
| dq473493-15 | VLV....... .......VPN IKESHSSTSN SAPALDAAET GHTSSVQPED |
| 124917-16 | VLV....... .......VPN INQSHPTTSN AAPVLDAAET GHTNKIQPED |
| hrv-18 | VLV....... .......VPN VNESHAITSN SAPALDAAET GHTSNVQPED |
| hrv-19 | VLV....... .......VPN INESHPSTSN AAPALDAAET GHTSNVQPED |
| hrv-20 | VLV....... .......VPN ITSSNSQTSN AAPALDAAET GHTNQVQPED |
| hrv-21 | VLV....... .......VPN IRESHGTTSN SAPALDAAET GHTSNVQPED |
| hrv-22 | VLV....... .......VPN TNESXPSTSN AAPALDAAET GHTSNVQPED |
| dq473497-23 | VLV....... .......VPN INSSHPTTSN SAPALDAAET GHTSNVQPED |
| hrv-24 | VLV....... .......VPN IKESKPSTSN SAPALDAAET GHTSSVQPED |
| ef173416-24 | VLV....... .......VPN IKESKPSTSN SAPALDAAET GHTSSVQPED |
| hrv-25 | VLV....... .......VPN IKESHPSTSN SAPILDAAET GHTSNVQPED |
| dq473508-28 | VLV....... .......VPN INPSNAQTTN AAPALDAAET GHTSQTQPED |
| hrv-29 | VLV....... .......VPN IRESHPSTSN SAPILDAAET GHTSNVQPED |
| dq473512-30 | VLV....... .......VPN INSSHPTTSN SAPALDAAET GHTSNVQPED |
| hrv-30 | VLV....... .......VPN INSSHPTTSN SAPALDAAET GHTSNVQPED |
| hrv-31 | VLV....... .......VPN IKESHPSTSN SAPILDAAET GHTSNVQPED |
| hrv-32 | VLV....... .......VPN IKESNPTTSN SAPALDAAET GHTSNVQPED |
| hrv-33 | VLV....... .......VPN IRESQATTSN SAPALDAAET GHTNNVQPED |
| hrv-34 | VLV....... .......VPN IKESQATTSN SAPALDAAET GHTSSVQPED |
| dq473501-34 | VLV....... .......VPN IKESQATTSN SAPALDAAET GHTSSVQPED |
| dq473505-36 | VLV....... .......VPN IQPSSSVSSH SAPALDAAET GHTSSVQPED |
| hrv-38 | VLV....... .......VPN IKESHSSTSN SAPALDAAET GHTSNVQPED |
| dq473495-38 | VLV....... .......VPN IKESHSSTSN SAPALDAAET GHTSNVQPED |
| ay751783-39 | VLV....... .......VPN IRESHPTTSN AATALDAAET GHTSSIQPED |
| hrv-40 | VLV....... .......VPN IRESHPTTSN SAPALDAAET GHTSNIQPED |
| dq473491-41 | VLV....... .......VPN ISESSPTTSN SAPALDAAET GHTSNVQPED |
| hrv-43 | VLV....... .......VPN TVESHSTTSN AAPALDAAET GHTSQVQPED |
| dq473499-44 | VLV....... .......VPN IRESHPSTSN SAPILDAAET GHTSNVQPED |
| hrv-45 | VLV....... .......VPN TRPSDGLIAN SAPALDAAET GHTSSVQPED |
| dq473506-46 | VLV....... .......VPN INASSGHTSN SAPALDAAET GHTSQIQPED |
| hrv-47 | VLV....... .......VPN IKESHPSTSN SAPILDAAET GHTSNVQPED |
| dq473496-49 | VLV....... .......VPN INSSHPTTSN SAPALDAAET GHTSNVQPED |
| hrv-49-f04 | VLV....... .......VPN INSSHPTTSN SAPALDAAET GHTSNVQPED |
| hrv-50 | VLV....... .......VPN IKESQATTSN SAPALDAAET GHTSNVQPED |
| hrv-51 | VLV....... .......VPN IQPSSGHTSN AAPALDAAET GHTSQVQPED |
| dq473507-53 | VLV....... .......VPN INASNPQTSN SAPALDAAET GHTSQTQPED |
| hrv-54 | VLV....... .......VPN IRESHPATSN SAPALDAAET GHTSGIQPED |
| hrv-54-f05 | VLV....... .......VPN IRESHPATSN SAPALDAAET GHTSGIQPED |
| dq473511-55 | VLV....... .......VPN IRESQAATSN SAPVLDAAET GHTSNVQPED |
| hrv-56 | VLV....... .......VPN IRESHPSTSN SAPALDAAET GHTSAIQPED |
| hrv-57 | VLV....... .......VPN IKQSQATTSN SAPALDAAET GHTSNVQPED |
| hrv-58 | VLV....... .......VPN IQPSTSVSSH SVPALDAAET GHTSSVQPED |
| dq473500-59 | VLV....... .......VPN IQESHPTTSN AAPALDAAET GHTSSIQPED |
| hrv-60 | VLV....... .......VPN IRESQPTTSN SAPALDAAET GHTSNVQPED |
| hrv-61 | VLV....... .......VPN INQSNPTTSN SAPVLDAAET GHTSNVQPED |
| hrv-62 | VLV....... .......VPN IKESHPSTSN SAPILDAAET GHTSNVQPED |
| hrv-63 | VLV....... .......VPN IQESHPTTSN AAPVLDAAET GHTSSIQPED |
| hrv-64 | VLI....... .......VPN INESHPSTSN AAPALDAAET GHTSNVQPED |
| ef173417-64 | VLI....... .......VPN INESHPSTSN AAPALDAAET GHTSNVQPED |
| hrv-65 | VLV....... .......VPN IQASNGHTSN AAPALDAAET GHTSQVQPED |
| hrv-66 | VLV....... .......VPN IKESHPSTSN AAPILDAAET GHTSKVQPED |
| hrv-67 | VLV....... .......VPN IKQSEPTTSN SAPALDAAET GHTSSVQPED |
| hrv-68 | VLV....... .......VPN IPASNTQTSN AAPALDAAET GHTNQVQPED |
| hrv-71 | VLV....... .......VPN IQPSNGHTSN SAPALDAAET GHTNQVQPED |
| dq473492-73 | VLV....... .......VPN INESNPTTSN SAPALDAAET GHTSGVQPED |
| dq473494-74 | VLV....... .......VPN IRESHSSTSN SAPALDAAET GHTSNVQPED |
| dq473510-75 | VLV....... .......VPN TMESHPTTSN AAPALDAAET GHTSHVQPED |
| dq473502-76 | VLV....... .......VPN IKESQATTSN AAPALDAAET GHTSSVQPED |
| hrv-76 | VLV....... .......VPN IKESQATTSN AAPALDAAET GHTSSVQPED |
| hrv-77 | VLV....... .......VPN TKESHPSTSN SAPILDAAET GHTSSVQPED |
| hrv-78 | VLV....... .......VPN IKESKPQSSN SAPVLDAAET GHTNQVQPED |
| ef173418-78 | VLV....... .......VPN IKESKPQSSN SAPVLDAAET GHTNQVQPED |
| hrv-80 | VLV....... .......VPN ISASNGHTSN SAPTLDAAET GHTSQVQPED |
| hrv-81 | VLV....... .......VPN INESHPTTSN SAPVLDAAET GHTSNIQPED |
| hrv-81-f06 | VLV....... .......VPN INESHPTTSN AAPVLDAAET GHTSNIQPED |
| hrv-81-f07 | VLV....... .......VPN INESHPTTSN AAPVLDAAET GHTSNIQPED |
| hrv-82 | VLV....... .......VPN INESHPSTSN SAPALDAAET GHTSTVQPED |
| dq473509-82 | VLV....... .......VPN INESHPSTSN SAPALDAAET GHTSTVQPED |
| hrv-85 | VLV....... .......VPN IKESHPTTSN SAPALDAAET GHTSSIQPED |
| pico-85 | VLV....... .......VPN IKESHPTTSN SAPALDAAET GHTSSIQPED |
| dq473504-88 | VLV....... .......VPN IQPSTSVSSH SVPALDAAET GHTSSVQPED |
| a10937-89 | VLV....... .......VPN IQPSTSVSSH AAPALDAAET GHTSSVQPED |
| hrv-89 | VLV....... .......VPN IQPSTSVSSH AAPALDAAET GHTSSVQPED |
| hrv-89-f08 | VLV....... .......VPN IQPSTSVSSH AAPALDAAET GHTSSVQPED |
| hrv-89-f09 | VLV....... .......VPN IQPSTSVSSH AAPALDAAET GHTSSVQPED |
| m16248-89 | VLV....... .......VPN IQPSTSVSSH AAPALDAAET GHTSSVQPED |
| hrv-90 | VLV....... .......VPN IKESKPSTSN SAPALDAAET GHTSDVQPED |
| hrv-94 | VLV....... .......VPN INESHPSTSN AAPALDAAET GHTSNVQPED |
| ef173419-94 | VLV....... .......VPN INESHPSTSN AAPALDAAET GHTSNVQPED |
| hrv-95 | VLV....... .......VPN TQASNGSIAN SAPALDAAET GHTSSVQPED |
| hrv-96 | VLV....... .......VPN INESYPTTSN SAPVLDAAET GHTSNVQPED |
| hrv-98 | VLV....... .......VPN IKESHPATSN SAPTLDAAET GHTSGIQPED |
| hrv-100 | VLV....... .......VPN IRESHPSTSN SAPILDAAET GHTSNVQPED |
| ef186077-qpm | VLV....... .......VPD TQASGPVHTT KPQALGAVEI GATADVGPET |
| ef077279-nat001 | VMA....... .......VPD TQASGPTHTN KPQTLGALEI GATAEIGPET |
| ny1078 | VLV....... .......VPD TQASGPVHTT KPQALGALEI GATADVGPEA |
| ef582385-c024 | VLQ....... .......VPN TQPSGPQHSI QPSALGAMEI GASSTTIPGD |
| ef077280-nat045 | VLV....... .......VPD TKPSGPTHTV KPTVLNAMEI GVTPDATPES |
| ef582387-c026 | VLV....... .......VPD TQASGPVHTT KPQALGAVEI GATADVGPET |
| ef582386-c025 | VLV....... .......VPN TQPSGPIHTT KPTALSAMEI GASSDVKPED |
| dq473485-03 | TKQ....... .......TLA SVSSGPKHTQ SVPALTANET GATLPTRPSD |
| ef173422-03 | TKQ....... .......TLA SVSSGPKHTQ SVPALTANET GATLPTRPSD |
| dq473490-04 | THQ....... .......TLA SVKSDSKHTQ KVPALTANET GATLPTTPSD |
| hrv-05 | AQQ....... .......TLA SIKSDSKHTQ KVPSLTANET GATLPTTPSD |
| dq473486-06 | TKQ....... .......TLA SVSSGPKHTQ SVPILTANET GATMPTNPSD |
| 105355-14 | TKQ....... .......TVA SISSGPKHTQ KVPILTANET GATMPVLPSD |
| k02121-14 | TKQ....... .......TVA SISSGPKHTQ KVPILTANET GATMPVLPSD |
| ef173420-17 | MKQ....... .......VTA SSQSGPKHTQ KVPALSANET GATLPTNPSD |
| hrv-26 | TQQ....... .......ALA SIKSGSKHTQ KAPALTANET GATMPTTPSD |
| hrv-27 | AKQ....... .......TIA SVSSNSKHTQ KVPTLSASET GTTLPTVPSD |
| ef173421-27 | AKQ....... .......TIA SVSSNSKHTQ KVPTLSASET GTTLPTVPSD |
| dq473487-35 | TKQ....... .......TVA SIASGSKHTQ SVPMLTANET GASMPVEPSD |
| hrv-35 | TKQ....... .......TVA SIASGSKHTQ SVPTLTANET GASMPVXPSD |
| ef173423-37 | TKQ....... .......TLA SISSGPKHTQ SVPTLTANET GATMPTNPSD |
| hrv-42 | TQQ....... .......TLA SVKSGSKHTQ KVPSLTANET GATLPTTPSD |
| dq473488-48 | MKQ....... .......ITA SSQSGPKHTQ KVPALSANET GATLPTNPSD |
| hrv-52 | MRQ....... .......ITA SSQSGPKHTQ KVPALSASET GATLPTNPSD |
| hrv-52-f10 | MRQ....... .......ITA SSQSGSKHTQ KVPALSASET GATLPTNPSD |
| ef173424-52 | MRQ....... .......ITA SSQSGPKHTQ KVPALSASET GATLPTNPSD |
| hrv-69 | MKQ....... .......VTA SVQSGSKHTQ KVPALSASET GATLPTNPSD |
| dq473489-70 | MKQ....... .......VTA SSQSGPKYTQ KVPALSANET GATLPTNPSD |
| hrv-72 | TKQ....... .......TLA SISSGPKYTQ SVPTLTANET GATMPTLPSD |
| hrv-79 | TRQ....... .......TLA SVSSGPKHTQ SVPTLTANET GATMPTQPSD |
| hrv-83 | TRQ....... .......TLA SVASGPKHTQ SVPILTANET GATMPTQPSD |
| hrv-84 | AKQ....... .......TIA SINSNSKHTQ QVPTLSASET GATLQTVPSD |
| hrv-86 | TKQ....... .......TLA SVATGSKYTQ KVPSLSANET GATMPTVPSD |
| hrv-91 | MKQ....... .......VTA SSQSGSKYTQ KVPALSANET GATLPTDPSD |
| hrv-92 | TKQ....... .......TLA SITSGPKHTQ SVPTLTANET GATMPTQPSD |
| ef173425-93 | AKQ....... .......TIA SVVSNSKHTQ KVPTLSASET GATLPTVPSD |
| hrv-97 | AKQ....... .......TIA SASSISKHTQ KVPTLSASET GATLPTVPSD |
| hrv-99 | TRQ....... .......TLA SIKSDSKHTQ KVPALTANET GATLPTKPSD |
| | 651 700 |
| annotation | HH........ ....HHHZHH HHH..BBBBB BBBBB..... .......... |
| af499637-cva13 | TVQTRHVIQT RSRSESTVES FFARGACVTI MGVDN.YNET LKG....... |
| af546702-cva21 | VVETRHVVNY KTRSESCLES FFGRAACVTI LSLTNSSKS. .......... |
| v01149-pv1m | TVQTRHVVQH RSRSESSIES FFARGACVTI MTVDNPASTT N......... |
| d00239-01 | AIETRYVMTS QTRDEMSIES FLGRSGCVHI SRIKVDYND. .........Y |
| pico-01 | AIETRYVITS QTRDEMSIES FLGRSGCVHI SRIKVDYTD. .........Y |
| hrv-01 | AIETRYVITS QTRDEMSIES FLGRSGCVHI SRIKVDYTD. .........Y |
| x02316-02 | VIETRYVQTS QTRDEMSLES FLGRSGCIHE SKLEVTLAN. .........Y |
| hrv-07 | MIETRYVITD QTRDETSIES FLGRSGCIAK VKLDTTQGD. .........Y |
| dq473503-07 | MIETRYVITD QTRDETSIES FLGRSGCIAK VKLDTTQGD. .........Y |
| hrv-08 | LIETRYVITD QTRHETSLES FLGRAGCIKI IALELDHDN. .........Y |
| pico-09 | MIETRYVQTS QTRDEMSLES FLGRSGCIHI SKLNIDYSN. .........Y |
| hrv-09 | MIETRYVQTS QTRDEMSLES FLGRSGCIHI SKLNIDYSD. .........Y |
| hrv-09-f01 | MIETRYVQTS QTRDEMSLES FLGRSGCIHI SKLNVDYDN. .........Y |
| hrv-09-f02 | MIETRYVQTS QTRDEMSLES FLGRSGCIHI SKLNVDYDN. .........Y |
| dq473498-10 | TVETRYVQTS QTRDEMSIES FLGRSGCIHT STITVNNTRP .........Y |
| hrv-10 | TVETRYVQTS QTRDEMSIES FLGRSGCIHT STITVNNTRP .........Y |
| ef173414-11 | MIETRYVQTS QTLDEMSIES FLGRSGCIHM SKLIVQYED. .........Y |
| ef173415-12 | VIETRYVITD QTRDEMSIES FLGRSGCISI IELDLDHEG. .........Y |
| hrv-13 | MVETRYVQTS QTRDEMSVES FLGRSGCIHM STMNIDYTN. .........Y |
| hrv-13-f03 | MVETRYVQTS QTRDEMSVES FLGRSGCIYM HTMNIDYDA. .........Y |
| dq473493-15 | MIETRYVQTS QTRDEMSIES FLGRSGCVHI SDLKIHYED. .........Y |
| 124917-16 | TIETRYVQSS QTLDEMSVES FLGRSGCIHE SVLDIVDN.. .........Y |
| hrv-18 | MIETRYVQTS QTRDEMSIES FLGRSGCIHI SKLVVHYED. .........Y |
| hrv-19 | MIETRYVQTS QTRDEMSIES FLGRSGCVHV SELQLDYTN. .........Y |
| hrv-20 | MVETRYVITD QTRDEMSVES FLGRSGCIAI IHTDLDHEAQ Q........Y |
| hrv-21 | MVETRYVQTS QTRDEMSIES FLGRSGCIHM SKLVVNYDN. .........Y |
| hrv-22 | MIETRYVLNS QTRDEMSIES FLGRSGCIHI SHLEVKYTG. .........Y |
| dq473497-23 | VIETRYVQTS QTRDEMSLES FLGRSGCIHE SKLKVEIGN. .........Y |
| hrv-24 | MVETRYVQTS QTLDEMSVES FLGRSGCIHM SKLTVDYDN. .........Y |
| ef173416-24 | MVETRYVQTS QTLHEMSVES FLGRSGCIHM SKLTVDYDN. .........Y |
| hrv-25 | TIETRYVQTT QTRDEMSIES FLGRSGCVHT STIETKLKH. .......... |
| dq473508-28 | MVETRYVITD QTRDEMSIES FLGRSSCIAI IHTDVVHETD K........Y |
| hrv-29 | TIETRYVQTS HTRDEMSIES FLGRSGCIHV STIKANQAH. .......... |
| dq473512-30 | VIETRYVQTS QTRDEMSLES FLGRSGCIHE SKLELELAH. .........Y |
| hrv-30 | VIETRYVQTS QTRDEMSLES FLGRSGCIHE SKLELELAR. .........Y |
| hrv-31 | TIETRYVQTS QTRDEMSIES FLGRSGCVHT SIIEPDGLH. .......... |
| hrv-32 | MIETRYVQTT HTRDEMSLES FLGRSGCVHI SKLDIDYTN. .........Y |
| hrv-33 | MIETRYVQTS QTLDEMSVES FLGRSGCIHM SKLVVKYED. .........Y |
| hrv-34 | MIETRYVQTS QTRDEMSIES FLGRSGCIHM SKLVVDYEN. .........Y |
| dq473501-34 | MIETRYVQTS QTRDEMSIES FLGRSGCIHM SKLVVDYEN. .........Y |
| dq473505-36 | MIETRYVITD QTRDETSIES FLGRSGCIAM IEFSTSSDKD EH........ |
| hrv-38 | MIETRYVQTS QTRDEMSVES FLGRSGCVHI SNLDIDYIN. .........Y |
| dq473495-38 | MIETRYVQTS QTRDEMSVES FLGRSGCVHI SNLDIDYIN. .........Y |
| ay751783-39 | TIETRYVQTS HTRDEMSVES FLGRSGCIHI STITMKKEN. .........Y |
| hrv-40 | TIETRFVQTS QTRDEMSIES FLGRSGCIHI STITVDNSLE .........Y |
| dq473491-41 | MIETRYVQTS QTRDEMSIES FLGRSGCVHK STLNIDYTD. .........Y |
| hrv-43 | MVETRSVHNF QTRDEMSIES FLGRSGCIHI STLEVNYDD. .........Y |
| dq473499-44 | TIETRYVQTS QTRDEMSIES FLGRSGCIHV STIKTNQAHN .......... |
| hrv-45 | LIETRYVIAD QTRHETSIES FLGRAGCVAT ISLDINHDD. .......... |
| dq473506-46 | MIETRYVITD QTKDEMSIES FLGRSGCIAI IETELNHEEG K........Y |
| hrv-47 | TIETRYVQTA QTRDEMSIES FLGRSGCVHS STIQSNEQH. .......... |
| dq473496-49 | VIETRYVQTS QTRDEMSLES FLGRSGCIHE SKLEVTLTN. .........Y |
| hrv-49-f04 | VIETRYVQTS QTRDEMSLES FLGRSGCIHE SKLEVTLTN. .........Y |
| hrv-50 | MLETRYVQTS QTRDEMSIES FLGRSGCIHI SKLVVDYDG. .........Y |
| hrv-51 | MVETRYVVTD QTRDEMSIES FLGRSACVAI IHTNLEHVEA DRQA.....Y |
| dq473507-53 | MLETRYVITD QTRDEMSIES FLGRSSCIAE IHTNLDHTG. .........Y |
| hrv-54 | TIETRFVQTS QTRDEMSIES FLGRAGCIHE STITIQNDVE .........Y |
| hrv-54-f05 | TIETRYVQTS QTRDEMSIES FLGRAGCIHE STITIRNDVE .........Y |
| dq473511-55 | VIETRYVQTS QTRDEMSIES FLGRSGCVHI SELVVHYEE. .........Y |
| hrv-56 | TIETRYVQTS QTRDEMSIES FLGRSGCIHI STITVDNDVD .........Y |
| hrv-57 | MIETRYVQTS QTRDEMSLES FLGRSGCIHI SELIVDYDN. .........Y |
| hrv-58 | MIETRYVITD QTRDETSIES FLGRSGCIAI IKFNTNKTN. .........Y |
| dq473500-59 | TIETRYVQTS QTRDEMSVES FLGRSGCIHI STITVNKDIK .........Y |
| hrv-60 | VIETRYVQIT QTRDEMSVES FLGRSGCIHI SKLEIDYSN. .........Y |
| hrv-61 | MIETRYVQTS QTRDEMSVES FLGRSGCIHM STLNINYDN. .........Y |
| hrv-62 | TIETRYVQTT QTRDEMSIES FLGRSGCVHT STIETTLSH. .......... |
| hrv-63 | TVETRYVQTS QTRDEMSVES FLGRSGCIHI STITVDKTID .........Y |
| hrv-64 | MIETRYVQNT QTRDEMSIES FLGRSGCIHI SDLKVNYTG. .........Y |
| ef173417-64 | MIETRYVQNT QTRDEMSIES FLGRSGCIHI SDLKVNYTG. .........Y |
| hrv-65 | VIETRYVITD QTRDEMSVES FLGRSACIAV IHTDLKHDHE GQNV.....Y |
| hrv-66 | TVETRYVQTS QTRDEMSIES FLGRSGCIHI STINVDSTKT .........Y |
| hrv-67 | MIETRYVQAS NTRDEMSLES FLGRSGCIHI SKLNIDYNA. .........Y |
| hrv-68 | VVETRYVITD QTRDEMSIES FLGRSGCIAI IHTDLDHNED Q........Y |
| hrv-71 | VMETRYVITD QTRDEMSIES FLGRSACIAT IHTKLVHGEE GV.......Y |
| dq473492-73 | MIETRYVQTS QTRDEMSIES FLGRSGCIHM STMNINYEN. .........Y |
| dq473494-74 | MVETRYVQTS QTRDEMSVES FLGRSGCIHI SHLKIDYTN. .........Y |
| dq473510-75 | MLETRQVQNF QTRDEMSIES FLGRSGCIHI STLEIDYTN. .........Y |
| dq473502-76 | MVETRYVQTS QTLDEMSMES FLGRSGCIHI SKLVVEYEG. .........Y |
| hrv-76 | MVETRYVQTS QTLDEMSMES FLGRSGCIHI SKLVVEYEG. .........Y |
| hrv-77 | TVETRYVQTS QTRDEMSIES FLGRSGCIHI STINVEDGKT .........Y |
| hrv-78 | TIETRYVITD QTRDEMSIES FLGRSGCATI MRLELDHTD. .........Y |
| ef173418-78 | TIETRYVITD QTRDEMSIES FLGRSGCATI MRLELDHTD. .........Y |
| hrv-80 | MIETRYVITD QTKDEMSIES FLGRSGCVAI IETKLNHETD M........Y |
| hrv-81 | TIETRYVQSS QTLDEMSVES FLGRSGCIHE SILDI.KED. .........Y |
| hrv-81-f06 | TIETRYVQSS QTLDEMSVES FLGRAGCIHE SILDI.KED. .........Y |
| hrv-81-f07 | TIETRYVQSS QTLDEMSVES FLGRAGCIHE SILDI.KED. .........Y |
| hrv-82 | MIETRYVQTS QTRDEMSLES FLGRSGCIHI SHLNVRYTD. .........Y |
| dq473509-82 | MIETRYVQTS QTRDEMSLES FLGRSGCIHI SHLNVKYTD. .........Y |
| hrv-85 | TIETRFVQTS QTRDEMSLES FLGRSGCIHI STITVNNNLD .........Y |
| pico-85 | TIETRFVQTS QTRDEMSLES FLGRSGCIHI STITVNNNLD .........Y |
| dq473504-88 | MIETRYVITD QTRDETSIES FLGRSGCIAK IKLDTNEGD. .........Y |
| a10937-89 | MIETRYVITD QTRDETSIES FLGRSGCIAM IEFNTSSDKT E......... |
| hrv-89 | MIETRYVITD QTRDETSIES FLGRSGCIAM IEFNTSSDKT E......... |
| hrv-89-f08 | MIETRYVITD QTRDETSIES FLGRSGCIAM IEFNTSSDKV E......... |
| hrv-89-f09 | MIETRYVITD QTRDETSIES FLGRSGCIAM IEFNTSSDKV E......... |
| m16248-89 | MIETRYVITD QTRDETSIES FLGRSGCIAM IEFNTSSDKT E......... |
| hrv-90 | VVETRYVQTS QTRDEMSVES FLGRSGCIHM SKLVVNYDN. .........Y |
| hrv-94 | MIETRYVQNT QTRDEMSIES FLGRSGCIHI AHLEIKYDG. .........Y |
| ef173419-94 | MIETRYVQNT QTRDEMSIES FLGRSGCIHI AHLEIKYDG. .........Y |
| hrv-95 | LIETRYVITD QTRYETSLES FLGRAGCIKI IALELDHDN. .......... |
| hrv-96 | MIETRYVQTS QTRDEMSIES FLGRSGCIHM STLNIDYDN. .........Y |
| hrv-98 | TIETRYVQTS QTRDEMSIES FLGRAGCIHE STITIQNDVE .........Y |
| hrv-100 | TVETRYVQTS QTRDEMSIES FLGRSGCIHT STITVSKMKN .........Y |
| ef186077-qpm | LIETRYVMND NTNAEAAVEN FLGRSA.LWA NLRLD..... .......... |
| ef077279-nat001 | LIETRYVMND NTNAEATVEN FLGRSA.LWA NFTLD..... .......... |
| ny1078 | MIETRYVMNS NTNAEAAVEN FLGRSA.LWA NLTLQ..... .......... |
| ef582385-c024 | LIETRYVINS NTNSEALIEN FMGRSA.LWA KIQVA..... .......... |
| ef077280-nat045 | VIETRYVINN HTNNEALMEN FLGRSS.LWA ELQMS..... .......... |
| ef582387-c026 | LIETRYVMND NTNAEATVEN FLGRSA.LWA NLKLN..... .......... |
| ef582386-c025 | MIETRYVVNS RTNGEATIEN FLGRSA.LWA NIDMA..... .......... |
| dq473485-03 | NVETRTTYMH FNGSETDVES FLGRAACVHV TEIKNKNAAG LD........ |
| ef173422-03 | NVETRTTYMH FNGSETDVES FLGRAACVHV TEIKNKNAAG LD........ |
| dq473490-04 | NVETRTTLMH YRGSETTLEN FLGRAACVHV VEIKNKR..V TDD......T |
| hrv-05 | SVETRTTLMH YTGSETTLEN FLGRAACVHV VEIVNKRPTD TEE....... |
| dq473486-06 | NVETRTTYMH FNGSETDVES FLGRAACVHI TEIENKNPAD IQ....... |
| 105355-14 | SIETRTTYMH FNGSETDVEC FLGRAACVHV TEIQNKDATG ID....... |
| k02121-14 | SIETRTTYMH FNGSETDVEC FLGRAACVHV TEIQNKDATG ID....... |
| ef173420-17 | NVETRTTYMH FTGSETSLEN FLGRSACVHI TEIQNKLPTE PRKEGDRMIN |
| hrv-26 | SVETRTTLMH YVGSETTLEN FLGRAACVHV VEIKNKEPQD V......... |
| hrv-27 | NIETRTTYMH YDGTETSLES FLGRAACVHV ITIENKLPTT GQ........ |
| ef173421-27 | NIETRTTYMH YDGTETSLES FLGRAACVHV ITIENKLPTT GQ........ |
| dq473487-35 | SVETRLTYMH FKGSETDVES FLGRAACVHM TEIVNKNPAG ST........ |
| hrv-35 | SVETRLTYMH FKGSETDVES FLGRAACVHM TEIVNKNPAX ST........ |
| ef173423-37 | NVETRTTYMH FNGSETDIES FLGRAACVHI TEIENKNSTG SV........ |
| hrv-42 | SVETRTTMMH YTGSETTLEN FLGRAACVHV VEIRNKRPTD GE........ |
| dq473488-48 | NVETRTTYMH FSGSETTLEN FLGRSACVHI TEIQNKRPEE FTSEETA..K |
| hrv-52 | NVETRTTYMH FTGSETTLEN FLGRSACVHI TEIENEIPQN PTPEGT...N |
| hrv-52-f10 | NVETRTTYMH FNGSETTLEN FLGRSACVHI TEIENKVPQD PTPEGG...N |
| ef173424-52 | NVETRTTYMH FTGSETTLEN FLGRSACVHI TEIENEIPQN PTPEGT...N |
| hrv-69 | SVETRTTYMH FTGSETTIEN FLGRSACVHI TEIQNKKPIE SLEEGK...N |
| dq473489-70 | NVETRTTYMH FTGSETSLEN FLGRSACVHI TEIKNKLPTE PVMKGNNMEN |
| hrv-72 | NVETRTTYMH FNGSETDIEC FLGRAACVHV TEIENKNPNG IS........ |
| hrv-79 | NVETRTTYMH FNGSETDVES FLGRAACVHM VKIVNKNPTD IV........ |
| hrv-83 | NVETRTTYMH FNGSETDIES FLGRAACVHM VEIVNKNPLN IK........ |
| hrv-84 | NVETRKTYMH YDGAETSVES FLGRSACVHV TEIENKSPAT DM........ |
| hrv-86 | NIETRTTYMN FTGSETDVEC FLGRAACVHI TEIENKDPTD IE........ |
| hrv-91 | NVETRTTYMH YTGSETTLEN FLGRSACVHI TEIQNKLPIK PTEEGGNMKN |
| hrv-92 | NVETRTTYMH FNGSETDVEN FLGRAACVHM VEIVNKNPEG LE........ |
| ef173425-93 | NVETRTTYMH YDGTETSLES FLGRAACVHV TDIENKLPTR ES........ |
| hrv-97 | NVETRTTYMH YDGTETSIES FLGRAACVHV TDIENKLPNT TD........ |
| hrv-99 | SVETRTTLMH YTGSETTLEN FLGRAACVHV VEIRNKRPTD EI........ |
| | 701 750 |
| annotation | HHHHHHH.BB CBB.....HH HAHHHHHHBB BBBBBBBDBB BBBB...... |
| af499637-cva13 | .DQKSTLFTT WNITYTDTVQ LRRKLEMFTY SRFDIEFTFV VTERYYSSNS |
| af546702-cva21 | .GEEKKHFNI WNITYTDTVQ LRRKLEFFTY SRFDLEMTFV FTENYPSTAS |
| v01149-pv1m | ...KDKLFAV WKITYKDTVQ LRRKLEFFTY SRFDMELTFV VTANFTETNN |
| d00239-01 | NGVN.KNFTT WKITLQEMAQ IRRKFELFTY VRFDSEVTLV PCIAG...RG |
| pico-01 | NGQD.INFTK WKITLQEMAQ IRRKFELFTY VRFDSEITLV PCIAG...RG |
| hrv-01 | NGQD.INFTK WKITLQEMAQ IRRKFELFTY VRFDSEITLV PCIAG...RG |
| x02316-02 | N..KE.NFTV WAINLQEMAQ IRRKFELFTY TRFDSEITLV PCISA...LS |
| hrv-07 | DTGKGVGFTT WKISLQEMAQ IRRKFELFTY TRFDSEITIV TAAAA...QG |
| dq473503-07 | DTGKGVGFTT WKISLQEMAQ VRRKFELFTY TRFDSEITIV TAAAA...QG |
| hrv-08 | DE....SFRT WGINIQEMSQ IRRKFEMFTY VRFDSEITIV PCIAA...IK |
| pico-09 | DK.SVENFTI WKINIKEMAQ LRRKFELFTY ARFDSEITLV PCIAA...ES |
| hrv-09 | DK.SVENFTI WKINIKEMAQ IRRKFELFTY ARFDSEITLV PCIAA...ES |
| hrv-09-f01 | DK.SVKNFTI WKINIKEMAQ IRRKFELFTY ARFDSEITLV PCIAA...ES |
| hrv-09-f02 | DK.SVKNFTI WKINIKEMAQ IRRKFELFTY ARFDSEITLV PCIAA...ES |
| dq473498-10 | NE...HTFDT WQITLQEMAQ IRRKFEMFTY VRFDSEVTLV PCIAG...KG |
| hrv-10 | NE...HTFDT WQITLQEMAQ IRRKFEMFTY VRFDSEVTLV PCIAG...KG |
| ef173414-11 | NG..KKNFNT WKINLQEMAQ IRRKFEMFTY TRFDSEITLV PCIAA...KG |
| ef173415-12 | SAE.GKNFKT WKINLKEMAQ IRRKNELFTY LRFDSEITIV PSNAA...IE |
| hrv-13 | DD.SVNNFVK WKISLQEMAQ VRRKFELFTY VRFDSEITIV PCIAG...QG |
| hrv-13-f03 | DD.SVNNFVK WKISLQEMAQ VRRKFELFTY VRFDSEITIV PCIAG...QG |
| dq473493-15 | NKD.GKNFTK WQINLKEMAQ IRRKFELFTY VRFDSEITLV PCIAA...KS |
| 124917-16 | NDQ...SFTK WNINLQEMAQ IRRKFEMFTY ARFDSEITMV PSVAA...KD |
| hrv-18 | NAE.TRNFVK WQINLQEMAQ IRRKFEMFTY VRFDSEITLV PSVAA...KG |
| hrv-19 | NQE.NNNFKT WQINLKEMAQ IRRKFELFTY LRFDSEVTLV PCIAA...KS |
| hrv-20 | NA.PGKNFSQ WKITIKEMAQ IRRKCELFTY LRFDSEITIV ATVAA...LG |
| hrv-21 | NT.GENNIST WQINIKEMAQ IRRKFEMFTY TRFDSEITLV PSIAA...RA |
| hrv-22 | NE.EGNNFNI WQINLKEMAQ IRRKFELFTY LRFDSEITLV PCIAS...QS |
| dq473497-23 | DE...NNFNT WNINLQEMAQ IRRKFELFTY TRFDSEITLV PCISA...LS |
| hrv-24 | DT...KNFFK WQINLQEMAQ VRRKFELFTY TRFDSEITIV PSIAG...KG |
| ef173416-24 | DT...KNFFK WQINLQEMAQ VRRKFELFTY TRFDSEITIV PSIAG...KG |
| hrv-25 | ...DER.FKT WNINLQEMAQ IRRKFEMFTY VRFDSEITLV PSIAG...RG |
| dq473508-28 | NH.PGKNFSK WNITLKEMAQ IRRKCEMFTY LRFDSEITIV VSVAS...KG |
| hrv-29 | ...DAK.FDK WNVNLQEMAQ IRRKFEMFTY VRFDSEITLV PCIAG...RG |
| dq473512-30 | DK...KNFTT WNINLQEMAQ IRRKFELFTY TRFDSEITLV PCISA...LS |
| hrv-30 | DK...KNFTT WNINLQEMAQ IRRKFELFTY TRFDSEITLV PCISA...LS |
| hrv-31 | ...DSK.YKV WHINLQEMAQ IRRKFEMFTY VRFDSEVTIV PCIAG...HG |
| hrv-32 | NK.SVKNFTI WKINIKEMAQ IRRKFELFTY TRFDSEITLV PCIAA...ES |
| hrv-33 | NE..KKNFMT WKINLQEMAQ IRRKFEMFTY ARFDSEITLV PSIAA...QG |
| hrv-34 | NA.KTKNFMT WQINLQEMAQ IRRKFEMFTY VRFDSEVTLV PSIAA...KG |
| dq473501-34 | NA.KTKNFMT WQINLQEMAQ IRRKFEMFTY VRFDSEVTLV PSIAA...KG |
| dq473505-36 | DK.IGKGFKT WKISLQEMAQ IRRKYELFTY TRFDSEITIV TAAAV...QG |
| hrv-38 | NS.EDKNFTT WQINLKEMAQ IRRKFEMFTY VRFDSEVTLV PCIAA...QN |
| dq473495-38 | NS.EDKNFTT WQINLKEMAQ IRRKFEMFTY VRFDSEVTLV PCIAA...QN |
| ay751783-39 | NE...HNFVD WKITLQEMAQ VRRKFEMFTY VRFDSEITLV PCIAG...RG |
| hrv-40 | DD...HHFDK WQITIQEMSQ IRRKFEFFTY ARFDSEITLV PCIAG...KG |
| dq473491-41 | DD.SIQNFKK WKISLQEMAQ IRRKFELFTY VRFDSEITIV PSIAG...QG |
| hrv-43 | NG.TGKNFTQ WPINLQEMAQ IRRKYELFTY LRFDSEITLV PCIAA...KS |
| dq473499-44 | ....TK.FDK WNINLQEMAQ IRRKFEMFTY VRFDSEITLV PCIAG...HG |
| hrv-45 | ...YQKNYKN WAISLQEMSQ IRRKFEMFTY VRFDSEITIV PCVAA...TE |
| dq473506-46 | NAED.QNFSK WKMTLLEMAQ IRRKCELFTY LRFDSEITIV TTLAG...QG |
| hrv-47 | ...INK.FKV WHINLQEMAQ IRRKYEMFTY VRFDSEVTMV PCIAG...YG |
| dq473496-49 | NE...NNFKV WNINLQEMAQ IRRKFELFTY TRFDSEITLV PCISA...LS |
| hrv-49-f04 | NE...NNFKV WNINLQEMAQ IRRKFELFTY TRFDSEITLV PCISA...LS |
| hrv-50 | NEE.TKNFKK WQINLQEMAQ IRRKFEMFTY VRFDSEVTLV PSIAA...KG |
| hrv-51 | NA.KGKNFST WKITLKEMAQ IRRKCELFTY LRFDSEITIV ATIAG...QG |
| dq473507-53 | NE.PGKNHSE WKITLKEMAQ IRRKCEMFTY LRFDSEITIV VSVAS...KQ |
| hrv-54 | ND...HHFKK WDITLQEMAQ IRRKFEFFTY VRFDSEITLV PCIAG...KG |
| hrv-54-f05 | ND...HHFKK WDITLQEMAQ IRRKFEFFTY VRFDSEITLV PCIAG...KG |
| dq473511-55 | NK.EGKNFTK WQVNIQEMAQ IRRKFEMFTY TRFDSEVTLV PSIAA...KG |
| hrv-56 | N...SKHYNK WQITLQEMAQ VRRKFEFFTY VRFDSEVTLV PCVAG...KG |
| hrv-57 | N...TKNFTE WKINLQEMAQ IRRKFELFTY VRFDSEVTLV PSIAA...QG |
| hrv-58 | DD.IGVGYKT WKISLQEMAQ IRRKFELFTY TRFDSEITIV TAAAA...QG |
| dq473500-59 | DD..GH.FLK WPITLQEVAQ IRRKFEFFTY VRFDSEITLV PCIAG...KG |
| hrv-60 | NE.ENKNFTT WQINLKEMAQ IRRKFELFTY VRFDSELTLV PCIAA...KN |
| hrv-61 | DD.SIENFKV WKINLQEMAQ IRRKFELFTY ARFDSEITIV PCVAG...QG |
| hrv-62 | ....KDRFKT WNINLQEMAQ IRRKFEMFTY VRFDSEITLV PSIAG...RG |
| hrv-63 | DT..GH.FNK WQITLQEMAQ IRRKFEFFTY VRFDSEVTLV PCIAG...KG |
| hrv-64 | NDE.GNNFNK WQITLKEMAQ IRRKYELFTY VRFDSEITLV PCISS...QS |
| ef173417-64 | NDE.GNNFNK WQITLKEMAQ IRRKYELFTY VRFDSEITLV PCISS...QS |
| hrv-65 | NDE.GRNFSA WEITLKEMAQ IRRKCELFTY LRFDSEITIV ATIAG...QG |
| hrv-66 | DE...SKFRT WQITLQEMAQ IRRKFEMFTY VRFDSEITLV PCIAG...KG |
| hrv-67 | DE.SRDNFTI WKINIKEMAQ IRRKFELFTY TRFDSEITLV PCIAA...ES |
| hrv-68 | NA.PGKNFSQ WKITIKEMAQ IRRKCELFTY LRFDSEITIV ATIAA...LG |
| hrv-71 | NM.KGNNLSK WQITLKEMAQ IRRKCELFTY LRFDSEITIV ATLAG...QG |
| dq473492-73 | DD.APENFTK WKISLQEMAQ IRRKFELFTY VRFDSEVTIV PCIAG...QS |
| dq473494-74 | NVE.GKNFTK WQINLKEMAQ IRRKFELFTY VRFDSEVTLV PCIAA...KS |
| dq473510-75 | NGE.GKNFTQ WPINLQEMAQ IRRKYELFTY LRFDSEVTLV PCIAA...KG |
| dq473502-76 | DD..TKNFKT WKINLQEMAQ VRRKFEMFTY TRFDSEVTLV PSIAA...KG |
| hrv-76 | DD..TKNFKT WKINLQEMAQ VRRKFEMFTY TRFDSEVTLV PSIAA...KG |
| hrv-77 | DE..SK.FRK WQITLQEMAQ IRRKFEMFTY VRFDSEITLV PCIAG...KG |
| hrv-78 | NA.EGKNFTT WKINLQEMAQ IRRKNEMFTY LRFDSEITLV CAVAS...QG |
| ef173418-78 | NAE.GKNFTT WKINLQEMAQ IRRKNEMFTY LRFDSEITLV CAVAS...QG |
| hrv-80 | NA.DGQNFSK WKITLMEMAQ IRRKCELFTY LRFDSEITIV TTLAG...QG |
| hrv-81 | NT...QSFTK WKINLQEMAQ IRRKFEMFTY TRFNSEITLV PSIAN...KE |
| hrv-81-f06 | NT...QSFTK WKINLQEMAQ IRRKFEMFTY TRFDSEITLV PSIAN...KE |
| hrv-81-f07 | NT...QSFTK WKINLQEMAQ IRRKFEMFTY TRFDSEITLV PSIAN...KE |
| hrv-82 | NE..GNNFRS WQISIKEMAQ IRRKFELFTY VRFDSEITLV PCIAS...QS |
| dq473509-82 | NEK..NNFRS WPISIKEMAQ IRRKFELFTY VRFDSEITLV PCIAS...QS |
| hrv-85 | DE...NHFDQ WQITIQEMAQ IRRKFEFFTY TRFDSEITLV PCIAG...KG |
| pico-85 | DE...NHFDQ WQITIQEMAQ IRRKFEFFTY TRFDSEITLV PCIAG...KG |
| dq473504-88 | DT.IGVGFVT WKISLQEMAQ IRRKFELFTY TRFDSEITIV TAAAA...QG |
| a10937-89 | HDKIGKGFKT WKVSLQEMAQ IRRKYELFTY TRFDSEITIV TAAAA...QG |
| hrv-89 | HDKIGKGFKT WKXSLQEMAQ IRRKYELFTY TRFDSEITIV TAAAA...QG |
| hrv-89-f08 | HDKIGKGFKT WKISLQEMAQ IRRKFELFTY TRFDSEITIV TAAAT...KG |
| hrv-89-f09 | HDKIGKGFKT WKISLQEMAQ IRRKFELFTY TRFDSEITIV TAAAT...KG |
| m16248-89 | HDKIGKGFKT WKVSLQEMAQ IRRKYELFTY TRFDSEITIV TAAAA...QG |
| hrv-90 | DE...NNFHK WQINLQEMAQ IRRKFELFTY ARFDSEITIV PSIAG...KG |
| hrv-94 | ND.AGNNFQS WQITLKEMAQ IRRKFELFTY VRFDSEITLV PCISS...QS |
| ef173419-94 | ND.AGNNFQS WQITLKEMAQ IRRKFELFTY VRFDSEITLV PCISS...QS |
| hrv-95 | ...YDKNFRT WGINIQEMSQ IRRKFEMFTY VRFDSEITIV PCIAA...IE |
| hrv-96 | DD.SPKNFKV WKINLQEMAQ IRRKFELFTY ARFDSEITIV PCVAV...QS |
| hrv-98 | ND...HHFRQ WDITLQEMAQ IRRKFEFFTY VRFDSEVTLV PCIAG...KG |
| hrv-100 | NE...HTFDK WQITLQEMAQ IRRKFEMFTY VRFDSEITLV PCIAG...KG |
| ef186077-qpm | .....QGFRK WEINFQEHAQ VRKKFEMFTY VRFDLEITIV T.....NNKG |
| ef077279-nat001 | .....RSFKS WKINFQEHAQ IRKKFELFTY VRFDLEITIV T.....NNRG |
| ny1078 | .....NSFVA WDINFQEHAQ VRKKFEMFTY VRFDLEITIV T.....NNAG |
| ef582385-c024 | .....NGFAK WDINFQEHAQ VRKKFEMFTY ARFDMEVTVV T.....NNTG |
| ef077280-nat045 | .....DGFKK WDINFQEQAH IRKKIEMFTY IRFDMEVTIV T.....NNQG |
| ef582387-c026 | .....QGFKE WKINFQEHAQ VRKKFEMFTY VRFDLEVTIV T.....NNTG |
| ef582386-c025 | .....NGYAT WSITYQGNAQ IRKKLELFTY VRFDLEITII T.....SNSS |
| dq473485-03 | NHRKEGLFND WKINLSSLVQ LRKKLELFTY VRFDSEYTIL ATAS.QPEAS |
| ef173422-03 | NHRKEGLFND WKINLSSLVQ LRKKLELFTY VRFDSEYTIL ATAS.QPEAS |
| dq473490-04 | KHKEELLFND WKISLSSLVQ LRRKLELFTY VRFDSEYTII ATSS.QPEDA |
| hrv-05 | .HRMQLLFNN WKINLSSLVQ LRRKLEMFTY VRFDSEYTII ATSS.QPNEA |
| dq473486-06 | NQKEEKLFND WKINFSSLVQ LRKKLELFTY IRFDSEYTIL ATAS.QP.KS |
| 105355-14 | NHREAKLFND WKINLSSLVQ LRKKLELFTY VRFDSEYTIL ATAS.QPDSA |
| k02121-14 | NHREAKLFND WKINLSSLVQ LRKKLELFTY VRFDSEYTIL ATAS.QPDSA |
| ef173420-17 | THKEQGLFND WKISLSSLVQ FRKKLELFTY VRFDSEYTIL ATAS.QPNTA |
| hrv-26 | NHKSELLFND WKINLSSLVQ LRRKIELFTY VRFDSEYTII ATSS.QPNNA |
| hrv-27 | THKEQKLYND WKINLSSLVQ LRRKLEMFTY VRFDSEYTII ATSS.QPQAA |
| ef173421-27 | THKEQKLYND WKINLSSLVQ LRRKLEMFTY VRFDSEYTII ATSS.QPQAA |
| dq473487-35 | NQKQDKLFND WRINLSSLVQ FRKKLELFTY VRFDSEYTIL ATAS.QPDNS |
| hrv-35 | NQKQDKLFND WRINLSSLVQ FRKKLELFTY VRFDSEYTIL ATAS.QPDNS |
| ef173423-37 | NHKSDKLFND WKINLSSLVQ LRKKLELFTY VRFDSEYTIL ATAS.QPSKS |
| hrv-42 | EHKTQLLFND WKINLSSLVQ LRRKLEMFTY VRFDSEYTII ATSS.QPNDA |
| dq473488-48 | THKEQKLFND WKISLSSLVQ LRKKLELFTY VRFDSEYTIL ATAS.QPDQA |
| hrv-52 | THKEQKLFSD WKVNLSSLVQ LRKKLELFTY VRFDSEYTIL ATAS.QPETA |
| hrv-52-f10 | THKEQKLFND WKISLSSLVQ LRKKLELFTY VRFDSEYTIL ATAS.QPEAA |
| ef173424-52 | THKEQKLFSD WKVNLSSLVQ LRKKLELFTY VRFDSEYTIL ATAS.QPETA |
| hrv-69 | THKGQMLFND WKISLSSLVQ LRKKLELFTY VRFDSEYTIL ATAS.QPNAS |
| dq473489-70 | THKEQGLFND WKINLSSLVQ FRKKLELFTY VRFDSEYTIL ATAS.QPDTA |
| hrv-72 | NHKAEKLFND WKISLSSLVQ LRKKLELFTY VRFDSEYTIL ATAS.QPDTA |
| hrv-79 | NQKEHLLFND WRINLSSLVQ LRKKLELFTY IRFDSEYTIL ATAS.QPNDS |
| hrv-83 | NQKREKLFNE WRINLSSLVQ LRKKLELFTY ARFDSEYTIL ATAS.QPTNS |
| hrv-84 | NHKNQKLFDD WKINLSSLVQ LRRKLEFFTY VRFDSEYTII ATSS.QPNDA |
| hrv-86 | NQKEAKLFND WKINLSSLVQ LRKKLELFTY VRFDSEYTIL ATAS.QPTQS |
| hrv-91 | THKEQKLFND WKINLSSLVQ FRKKLELFTY VRFDSEYTIL ATAS.QPETA |
| hrv-92 | NQKEHKLFND WRINLSSLVQ LRKKLELFTY VRFDSEYTIL ATAS.QPTSS |
| ef173425-93 | THKEQKLYND WKINLSSLVQ LRRKLDMFTY VRFDSEYTII ATSS.QPQDA |
| hrv-97 | NHKAQKLFND WKINLSSLVQ LRRKLDMFTY VRFDSEYTII ATSS.QPERA |
| hrv-99 | EHKKDLLFND WKINLSSLVQ LRRKLELFTY VRFDSEYTII ATSS.QPDGA |
| | 751 800 |
| annotation | .....BBBEB BB........ ....HHBHH. ....BBFBBB .BB.BBBBG. |
| af499637-cva13 | GHALNQVYQI MYVPPGAPVP KKWDDYTWQT SSNPSIFYTY GSAPPRISIP |
| af546702-cva21 | GEVRNQVYQI MYIPPGAPRP SSWDDYTWQS SSNPSIFYMY GNAPPRMSIP |
| v01149-pv1m | GHALNQVYQI MYVPPGAPVP EKWDDYTWQT SSNPSIFYTY GTAPARISVP |
| d00239-01 | DDIGHVVMQY MYVPPGAPIP KTRNDFSWQS GTNMSIFWQH GQPFPRFSLP |
| pico-01 | DDIGHIVMQY MYVPPGAPIP SKRNDFSWQS GTNMSIFWQH GQPFPRFSLP |
| hrv-01 | DDIGHIVMQY MYVPPGAPIP SKRNDFSWQS GTNMSIFWQH GQPFPRFSLP |
| x02316-02 | QDIGHITMQY MYVPPGAPVP NSRDDYAWQS GTNASVFWQH GQAYPRFSLP |
| hrv-07 | NDIGHIVMQF MYVPPGAPVP IKREDYTWQS GTNASIFWQE GQPYPRFTIP |
| dq473503-07 | NGIGHIVMQF MYAPPGAPVP IKREDYTWQS GTNASIFWQE GQPYPRFTIP |
| hrv-08 | GDLGHIVLQY MYVPPGAPLP DKRMHDAWQT STNASVFWQV GQTYPRFTIP |
| pico-09 | ESVGHVVMQY MYVPPGAPLP RTRDDYAWQS GTNASIFWQH GQSYPRFSLP |
| hrv-09 | DSVGHVVMQY MYVPPGAPLP RTRDDYAWQS GTNASIFWQH GQSYPRFSLP |
| hrv-09-f01 | ESVGHVVMQY MYVPPGAPLP RTRDDYAWQS GTNASIFWQH GQSYPRFSLP |
| hrv-09-f02 | ESVGHVVMQY MYVPPGAPLP RTRDDYAWQS GTNASIFWQH GQSYPRFSLP |
| dq473498-10 | DDIGHIVMQY MYVPPGAPVP TKRDDFAWQS GTNASVFWQH GQPFPRFSLP |
| hrv-10 | DDIGHIVMQY MYVPPGAPVP TKRDDFAWQS GTNASVFWQH GQPFPRFSLP |
| ef173414-11 | NDIGHVVMQY MYVPPGAPVP EKRDDYTWQS GTNASIFWQY GQTYPRFSLP |
| ef173415-12 | GSNGHVVVQY MYVPPGAPLP KKRDDYTWQS GTNASVFWQE GQPYPRFTIP |
| hrv-13 | GDVGHVVMQY MYVPPGAPLP TKRNDYTWQS GTNASVFWQH GQIYPRFSLP |
| hrv-13-f03 | GDVGHVVMQY MYVPPGAPLP TKRNDYTWQS GTNASVFWQH GQIYPRFSLP |
| dq473493-15 | DNIGHVVMQY MYVPPGAPLP NKRNDYTWQS GTNASVFWQH GQPYPRFSLP |
| l24917-16 | GHIGHIVMQY MYVPPGAPIP TTRDDYAWQS GTNASVFWQH GQPFPRFSLP |
| hrv-18 | DDIGHIVMQY MYVPPGAPIP KTRDDFAWQS GTNASIFWQH GQTYPRFSLP |
| hrv-19 | KNIGHVVMQY MYVPPGAPIP KTRNDYTWQS GTNASIFWQH GQPYPRFSLP |
| hrv-20 | RDNGHVVLQY MYVPPGAPIP KTRDDYTWQS GTNASVFWQQ GQPYPRFTIP |
| hrv-21 | GDIGHVVMQY MYVPPGAPIP KTREDFAWQS GTNASIFWQH GQTYPRFSLP |
| hrv-22 | KNIGHVVMQY MYVPPGAPKP EKRDDYTWQS GTNASVFWQH GQPYPRFSLP |
| dq473497-23 | QDIGHITMQY MYVPPGAPIP KSRNDYAWQS GTNASIFWQH GQTYPRFSLP |
| hrv-24 | DDIGHVVMQY MYIPPGAPVP TKRDDYTWQS GTNASVFWQH GQTYPRFSLP |
| ef173416-24 | DDIGHVVMQY MYIPPGAPVP TKRDDYTWQS GTNASVFWQH GQTYPRFSLP |
| hrv-25 | ADIGHIVMQY MYVPPGAPLP TDRKHFAWQS STNASIFWQH GQPFPRFSLP |
| dq473508-28 | DDNGHVVMQY MYVPPGAPIP TTRNDYTWQS STNASVFWQQ GQPYPRFTIP |
| hrv-29 | NDIGHIVMQY MYVPPGAPVP NDRNHFAWQS GTNASIFWQH GQPFPRFSLP |
| dq473512-30 | QDIGHITMQY MYVPPGAPIP ESRNDYAWQS GTNASVFWQH GQTYPRFSLP |
| hrv-30 | QDIGHITMQY MYVPPGAPIP ESRNDYAWQS GTNASVFWQH GQTYPRFSLP |
| hrv-31 | SDIGHIVMQY MYVPPGAPVP TNRKHFAWQS GTNASIFWQH GQPFPRFTLP |
| hrv-32 | DSIGHVVMQY MYVPPGAPLP QARDDYTWQS GTNASIFWQH GQAYPRFSLP |
| hrv-33 | DDVGHVVMQY MYVPPGAPAP EKRDDYTWQS GTNASVFWQY GQTYPRFSLP |
| hrv-34 | DDIGHVVMQY MYVPPGAPIP KTRDDFAWQS GTNASIFWQH GQTYPRFSLP |
| dq473501-34 | DDIGHVVMQY MYVPPGAPIP KTRDDFAWQS GTNASIFWQH GQTYPRFSLP |
| dq473505-36 | DDSGHVVLQF MYVPPGAPVP VKRDDYTWQS GTNASVFWQE GQPYPRFTIP |
| hrv-38 | EGVGHVVMQY MYVPPGAPLP RKRDDYTWQS GTNASVFWQY GQTYPRFSLP |
| dq473495-38 | EGVGHVVMQY MYVPPGAPLP RKRDDYTWQS GTNASVFWQY GQTYPRFSLP |
| ay751783-39 | EDIGHIVMQY MYVPPGAPVP KKRDDYTWQS GTNASVFWQH GQPYPRFSLP |
| hrv-40 | EDIGHIVMQY MYVPPGAPIP KKRNDYTWQS GTNASVFWQH GQTFPRFSLP |
| dq473491-41 | SDVGHVVMQY MFVPPGAPLP EKRDDYTWQS GTNASVFWQY GQVYPRFSLP |
| hrv-43 | NDIGHVVMQY MYVPPGAPIP KTRKDYAWQS GTNASVFWQH GQTFPRFSLP |
| dq473499-44 | DDIGHIVMQY MYVLPGAPVP DDRNHFAWQS GTNASIFWQH GQPFPRFSLP |
| hrv-45 | GNLGHIVVQY MFVPPGAPLP VSRTDNTWQS STNASVFWQV GQTYPRFSIP |
| dq473506-46 | DDIGHVVIQY MYVPPGAPLP RYRNDYTWQS GTNASVFWQQ GQPYPRFTIP |
| hrv-47 | DDIGHIVMQY MYVPPGAPVP TSREHFAWQS GTNASIFWQQ GQPFPRFSLP |
| dq473496-49 | KDIGHITMQY MYVPPGAPVP KSRDDYAWQS GTNASVFWQH GQAYPRFSLP |
| hrv-49-f04 | EDIGHITMQY MYVPPGAPVP KSRDDYAWQS GTNASIFWQH GQAYPRFSLP |
| hrv-50 | VDIGHVVMQY MYVPPGAPIP KTRDDFAWQS GTNASIFWQH GQTYPRFSLP |
| hrv-51 | DDIGHIVLQY MYVPPGGPVP LTRKDDEWQS GTNASVFWQH GQPYPRFTIP |
| dq473507-53 | GNNGHVVIQY MYVPPGAPIP KTRDDYTWQS GTNASVFWQQ GQPYPRFTIP |
| hrv-54 | VDIGHIVMQF MYVPPGAPKP EKRNDYTWES STNPSIFWQH GQAYPRFSLP |
| hrv-54-f05 | ADIGHIVMQF MYVPPGAPKP EKRNDYTWES STNPSIFWQH GQAYPRFSLP |
| dq473511-55 | DDIGHVVMQY MYVPPGAPIP KTREDYAWQS GTNASIFWQH GQTYPRFSLP |
| hrv-56 | DDIGHIVMQY MYVPPGAPLP NSRDDFTWQS GTNASVFWQH GQAFPRFSLP |
| hrv-57 | EDVGHVVMQY MYVPPGAPIP KTRKDYAWQS GTNASIFWQH GQTYPRFSLP |
| hrv-58 | EDNGHIVLQF MYVPPGAPVP KNRDDFTWQS GTNASVFWQE GQPYPRFTIP |
| dq473500-59 | DDIGHIVMQY MYVPPGAPLP TKRDDYTWQS GTNASIFWQH GQTFPRFSLP |
| hrv-60 | DGIGHVVMQY MYVPPGAPLP TKRDDYTWQS GTNASVFWQH GQTYPRFSLP |
| hrv-61 | GDIGHVVMQY MYVPPGAPTP EKRNDFTWQS GTNASVFWQH GQAYPRFSLP |
| hrv-62 | ADIGHIVMQY MYVPPGAPLP TDRKHFAWQS STNASIFWQH GQPFPRFSLP |
| hrv-63 | DDIGHIVMQY MYVPPGAPLP TRREDYTWQS GTNASIFWQH GQAFPRFSLP |
| hrv-64 | ANIGHVVMQY MYVPPGAPIP TKRNDYVWQS GTNASIFWQH GQPYPRFSLP |
| ef173417-64 | ANIGHVVMQY MYVPPGAPIP TKRNDYVWQS GTNASIFWQH GQPYPRFSLP |
| hrv-65 | DDIGHIVLQY MYVPPGGPVP KTRKDEEWQT GTNASVFWQH GQPYPRFTIP |
| hrv-66 | DDIGHIVMQY MYVPPGAPIP DNRTHFAWQS GTNASIFWQL GQPFPRFSLP |
| hrv-67 | DSIGHVVMQY MYVPPGAPLP TKRDDYTWQS GTNASIFWQH GQSYPRFSLP |
| hrv-68 | KDNGHVVLQY MYVPPGAPIP KTRDDYTWQS GTNASVFWQQ GQPYPRFTIP |
| hrv-71 | DDLGHIALQY MYVPPGGPIP ETRDADEWQS GTNASVFWQH GQPYPRFTIP |
| dq473492-73 | GDVGHVVMQY MYVPPGAPLP TKRNDYTWQS GTNASVFWQH GQTYPRFSLP |
| dq473494-74 | DNIGHVVMQY MYVPPGAPLP KKRDDYTWQS GTNASVFWQH GQPYPRFSLP |
| dq473510-75 | NDIGHVVMQY MYVPPGAPIP TTRKDYAWQS GTNASVFWQH GQTFPRFSLP |
| dq473502-76 | DDIGHVVMQY MYVPPGAPVP KKRDDYTWQS GTNASIFWQY GQTYPRFSLP |
| hrv-76 | DDIGHVVMQY MYVPPGAPVP KKRDDYTWQS GTNASIFWQY GQTYPRFSLP |
| hrv-77 | DDIGHVVMQY MYVPPGAPIP DSRTHFAWQS GTNASIFWQH GQPFPRFSLP |
| hrv-78 | DNNGHVVLQF MFVPPGAPIP KKRDDYTWQS GTNASVFWQQ GQTYPRFSIP |
| ef173418-78 | DNNGHVVLQF MFVPPGAPIP KKRDDYTWQS GTNASVFWQQ GQTYPRFSIP |
| hrv-80 | EDIGHVVIQY MYVPPGAPLP NKRNDYTWQS GTNASVFWQQ GQPYPRFTIP |
| hrv-81 | GHIGHIVMQY MYVPPGAPIP TTREDYAWQS GTNASIFWQH GQPFPRFSLP |
| hrv-81-f06 | GHIGHIVMQY MYVPPGAPIP TTREDYAWQS GTNASIFWQH GQPFPRFSLP |
| hrv-81-f07 | GHIGHIVMQY MYVPPGAPIP TTREDYAWQS GTNASIFWQH GQPFPRFSLP |
| hrv-82 | NDIGHVVMQY MYVPPGAPKP EKRDDYTWQS GTNASIFWQH GQPYPRFSLP |
| dq473509-82 | NDIGHVVMQY MYVPPGAPKP EKRDDYTWQS GTNASIFWQH GQPYPRFSLP |
| hrv-85 | DDIGHIVMQY MYVPPGAPIP RKRDDYTWQS GTNASVFWQH GQTFPRFSLP |
| pico-85 | DDIGHIVMQY MYVPPGAPIP RKRDDYTWQS GTNASVFWQH GQTFPRFSLP |
| dq473504-88 | DDTGHIVLQF MYVPPGAPIP KKRDDYTWQS GTNASVFWQE GQPYPRFTIP |
| a10937-89 | NDSGHIVLQF MYVPPGAPVP EKRDDYTWQS GTNASVFWQE GQPYPRFTIP |
| hrv-89 | XDSGHIVLQF MYVPPGAPVP EKRDDYTWQS GTNASVFWQE GQPYPRFTIP |
| hrv-89-f08 | DDNGHIVLQF MYVPPGAPVP EKRDDYTWQS GTNASVFWQE GQPYPRFTIP |
| hrv-89-f09 | DDNGHIVLQF MYVPPGAPVP EKRDDYTWQS GTNASVFWQE GQPYPRFTIP |
| m16248-89 | NDSGHIVLQF MYVPPGAPVP EKRDDYTWQS GTNASVFWQE GQPYPRFTIP |
| hrv-90 | NDIGHVVMQY MYIPPGAPVP EKRDDYAWQS GTNASIFWQH GQTYPRFSLP |
| hrv-94 | ANIGHVVMQY MYVPPGAPKP DKRDDYVWQS GTNASIFWQH GQPYPRFSLP |
| ef173419-94 | ANIGHVVMQY MYVPPGAPKP DKRDDYVWQS GTNASIFWQH GQPYPRFSLP |
| hrv-95 | GDLGHIVLQY MYVPPGAPLP DKRMHDAWQT STNASVFWQV GQTYPRFTIP |
| hrv-96 | GDIGHVVMQY MYVPPGAPTP EKRDDFTWQS GTNASVFWQH GQAYPRFSLP |
| hrv-98 | ADIGHIVMQF MYVPPGAPKP KKRNDYTWES STNPSIFWQH GQAYPRFSLP |
| hrv-100 | DDIGHIVMQY MYVPPGAPVP TKRDDFAWQS GTNASVFWQH GQPFPRISLP |
| ef186077-qpm | ......LMQI MFVPPGITPP GGKDGREWDT ASNPSVFFQP NSGFPRFTIP |
| ef077279-nat001 | ......LMQV MFVPPGIDVP KQKEGKEWNA ASNPSVFFQP HSGFPRFTIP |
| ny1078 | ......LMQI MYVPPGARAP ANKDSKEWDS ASNPSVFYQP HSGFPRFTIP |
| ef582385-c024 | ......LVQI MFVPPGIDAP DSIDSRLWDS ASNPSVFYQP KSGFPRFTIP |
| ef077280-nat045 | ......LMQI MYVPPGIEAP ESLNDKRWNG ASNPSVFYQP KSGFPRFTIP |
| ef582387-c026 | ......LMQI MFIPPGIPAP NGKNSREWDT ASNPSVFFQP NSGFPRFTIP |
| ef582386-c025 | ......LMQI MYVPPGAKPP NNNSSMEWNT ASNPSIFFQP GNGFPRFTIP |
| dq473485-03 | SYSSNLTVQA MYVPPGAPNP KEWDDYTWQS ASNPSVFFKV GET.SRFSVP |
| ef173422-03 | SYSSNLTVQA MYVPPGAPNP KEWDDYTWQS ASNPSVFFKV GET.SRFSVP |
| dq473490-04 | SYSSNLTVQA MFIPPGAPNP IKWDDYTWQS ATNPSVFFNV GKS.ARFSVP |
| hrv-05 | KFSSNLTIQA MFIPPGAPNP KKWDDYTWQS ATNPSVFFNV GKS.ARFSVP |
| dq473486-06 | NYASNLVVQA MYVPPGAPNP EKWDDFTWQS ASNPSVFFKV GDT.SRFSVP |
| l05355-14 | NYSSNLVVQA MYVPPGAPNP KEWDDYTWQS ASNPSVFFKV GDT.SRFSVP |
| k02121-14 | NYSSNLVVQA MYVPHGAPKS KRVGDYTWQS ASNPSVFFKV GDT.SRFSVP |
| ef173420-17 | KYASNLTVQA MYVPPGAPNP VKWNDYTWQS ASNPSVFFEV GKM.ARFSVP |
| hrv-26 | KYSSNLTVQA MYIPPGAPNP VKWDDYTWQS ATNPSVFFNV GKS.ARFSVP |
| hrv-27 | RFSNTLTVQA MFIPPGAPNP VEWDDYTWQS ASNPSVFFNV GKS.ARFSVP |
| ef173421-27 | RFSNTLTVQA MFIPPGAPNP VEWDDYTWQS ASNPSVFFNV GKS.ARFSVP |
| dq473487-35 | KYSSNLTVQA MYVPPGAPNP EAWNDYTWQS ASNPSVFFKV GDT.SRFSVP |
| hrv-35 | KYSSNLTVQA MYVPPGAPNP EAWNDYTWQS ASNPSVFFKV GDT.SRFSVP |
| ef173423-37 | NYASNLVVQA MYVPPGAPNP KEWNDFTWQS ASNPSVFFKV GDT.ARFSVP |
| hrv-42 | KFSSNLTVQA MFIPPGAPNP SKWDDYTWQS ATNPSVFFNV GKS.ARFSVP |
| dq473488-48 | QYASNLIVQA MYVPPGAPNP IEWNDYTWQS ASNPSVFFEV GKT.ARFSVP |
| hrv-52 | QYASNLTVQA MYVPPGAPNP VEWNDYTWQS ASNPSVFFEV GKT.ARFSVP |
| hrv-52-f10 | EYASNLTVQA MYVPPGAPNP VKWNDYTWQS ASNPSVFFEV GKT.ARFSVP |
| ef173424-52 | QYASNLTVQA MYVPPGAPNP VEWNDYTWQS ASNPSVFFEV GKT.ARFSVP |
| hrv-69 | EYASNLTVQA MYVPPGAPNP VKWNDYTWQS ASNPSVFFEV GKT.ARFSVP |
| dq473489-70 | QYASNLTVQA MYVPPGAPNP IKWDDYTWQS ASNPSVFFEV GKM.ARFSVP |
| hrv-72 | NYSSNLVVQA MYVPPGAPNP VEWDDYTWQS ASNPSVFFKV GDT.SRFSVP |
| hrv-79 | QYSSNLTVQA MYVPPGAPNP TKWDDYTWQS ASNPSVFFKV GDT.ARFSVP |
| hrv-83 | SYSSDLTVQA MYVPPGAPNP TKWDDYTWQS ASNPSVFFKV GDT.ARFSVP |
| hrv-84 | QYSNTLTIQA MFIPPGAPNP KEWDDYTWQS ASNPSVFFNV GNS.ARFSVP |
| hrv-86 | SYSSNLTVQA MYVPPGAPNP KTWNDYTWQS ASNPSVFFKV GDT.ARFSVP |
| hrv-91 | QYASNLTVQA MYVPPGAPNP IKWDDYTWQS ASNPSVFFEV GKM.ARFSVP |
| hrv-92 | KYSSSLTVQA MYVPPGAPNP TKWDDYTWQS ASNPSVFFKV GDT.ARFSVP |
| ef173425-93 | QFSNTLTVQA MFIPPGAPNP QEWDDYTWQS ASNPSVFFNV GKS.ARFSVP |
| hrv-97 | QFSNTLTVQA MFIPPGAPNP IQWDDYTWQS ASNPSVFFNV GRS.ARFSVP |
| hrv-99 | KYSSNLTIQA MFIPPGAPNP IKWDDYTWQS ATNPSVFFNV GKT.ARFSVP |
| | 801 850 |
| annotation | .......BBB BLLLLLL... .....LLLLL LLLLLHHHHL .BBHBBB... |
| af499637-cva13 | FVGIANAYSH FYDGYATVPL KTDTTDSGAA YYGAVSINDF GLLAVRVVNE |
| af546702-cva21 | YVGIANAYSH FYDGFARVPL EGENTDAGDT FYGLVSINDF GVLAVRAVNR |
| v01149-pv1m | YVGISNAYSH FYDGFSKVPL KDQSAALGDS LYGAASLNDF GILAVRVVND |
| d00239-01 | FLSIASAYYM FYDGYDG... .....DNSSS KYGSIVTNDM GTICSRIVTE |
| pico-01 | FLSIASAYYM FYDGYDG... .....DNTSS KYGSVVTNDM GTICSRIVTE |
| hrv-01 | FLSIASAYYM FYDGYDG... .....DNTSS KYGSVVTNDM GTICSRIVTE |
| x02316-02 | FLSVASAYYM FYDGYD.... .....EQDQ. NYGTANTNNM GSLCSRIVTE |
| hrv-07 | FMSIASAYYM FYDGYDG... .....DDASS KYGSVVTNDM GTICVRLVTS |
| dq473503-07 | FMSIASAYYM FYDGYDG... .....DDASS KYGSVVTNDM GTICVRLVTS |
| hrv-08 | FSSIASAYYM FYDGYDS... .....DGLDA IYGIPVTNHM GTICVRMVTD |
| pico-09 | FLSIASAYYM FYDGYDG... ......GPDS QYGTIVTNDM GSLCSRIVTE |
| hrv-09 | FLSIASAYYM FYDGYDG... ......GPDS LYGTIVTNDM GSLCSRVVTE |
| hrv-09-f01 | FLSIASAYYM FYDGYDG... ......GPDS QYGTIVTNDM GSLCSRIVTE |
| hrv-09-f02 | FLSIASAYYM FYDGYDG... ......GPDS QYGTIVTNDM GSLCSRIVTE |
| dq473498-10 | FLSIASAYYM FYDGYDG... .....DTHDS RYGTTVINHM GTLCMRIVTN |
| hrv-10 | FLSIASAYYM FYDGYDG... .....DTHDS RYGTTVINHM GTLCMRIVTN |
| ef173414-11 | FMSIASAYYM FYDGYDG... .....DQPNS RYGNIVTNDM GTLCYRIVTD |
| ef173415-12 | FISIASAYYM FYDGYAD... .....DNPSA PYGTVVTNDM GSLCVRIVTD |
| hrv-13 | FLSIASAYYM FYDGYNG... .....DSSDA RYGTTITNDM GTLCFRIVTE |
| hrv-13-f03 | FLSIASAYYM FYDGYNG... .....DSTDA RYGTTVTNDM GTLCFRIVTE |
| dq473493-15 | FLSIASAYYM FYDGYDG... .....DSTES HYGTVVTNDM GTLCSRIVTE |
| l24917-16 | FLSIASAYYM FYDGYDG... .....DTYKS RYGTVVTNDM GTLCSRIVTS |
| hrv-18 | FLSIASAYYM FYDGYDG... .....DQTSS RYGTVATNDM GTLCSRIVTD |
| hrv-19 | FLSIASAYYM FYDGYDG... .....DSPGS RYGTIVTNDM GTLCSRIVTD |
| hrv-20 | FMSIASAYYM FYDGYE.... .....DDKGS VYGSVVTNDM GTLCVRIVTE |
| hrv-21 | FLSIASAYYM FYDGYDG... .....DQTDS QYGAVVTNDM GSLCYRIVTG |
| hrv-22 | FLSVASAYYX FYDGYDG... ......GPGS RYGAVVTNDM GTLCSRIVTE |
| dq473497-23 | FLSVASAYYM FYDGYN.... ......EKGT HYGTVSTNNM GTLCSRVVTE |
| hrv-24 | FLSVASAYYM FYDGYDG... .....DQHDS VYGSVVTNDM GTLCYRIVTD |
| ef173416-24 | FLSVASAYYM FYDGYDG... .....DQHDS VYGSVVTNDM GTLCYRIVTD |
| hrv-25 | FLSVASAYYM FYDGYNG... .....DDHTA RYGTTVVNRM GALCMRIVTN |
| dq473508-28 | FMSIASAYYM FYDGYE.... .....DDNGT TYGAVVTNHM GTLCARIVTE |
| hrv-29 | FLSVASAYYM FYDGYNG... .....GDHTA TYGTTVVNRM GTLCVRIVTG |
| dq473512-30 | FLSVASAYYM FYDGYN.... ......EGGT NYGTVSTNNM GTLCSRVVTE |
| hrv-30 | FLSVASAYYM FYDGYN.... ......EGGT NYGTVSTNNM GTLCSRVVTE |
| hrv-31 | FLSVASAYYM FYDGYDG... .....DKSGA KYGTTVVNRM GALCMRVVTN |
| hrv-32 | FLSIASAYYM FYDGYDG... ......GPDS QYGTIVTNDM GSLCSRIVTE |
| hrv-33 | FLSIASAYYM FYDGYDG... .....DQPNS RYGNMVTNDM GTLCSRIVTD |
| hrv-34 | FLSIASAYYM FYDGYDG... .....DQHDS RYGTVVTNDM GTLCSRIVTD |
| dq473501-34 | FLSIASAYYM FYDGYDG... .....DQHDS RYGTVVTNDM GTLCSRIVTD |
| dq473505-36 | FMSIASAYYM FYDGYDG... .....DNAAS KYGSVVTNDM GTICVRIVTS |
| hrv-38 | FLSIASVYYM FYDGYDG... .....DSTES HYGTAVTNDM GTLCSRIVTE |
| dq473495-38 | FLSIASAYYM FYDGYDG... .....DSTES HYGTAVTNDM GTLCSRIVTE |
| ay751783-39 | FLSIASAYYM FYDGYDG... .....DKSSS RYGVSVTNDM GTLCTRIVTN |
| hrv-40 | FLSIASAYYM FYDGYDG... .....DTSTS RYGTSVTNHM GTLCSRIVTN |
| dq473491-41 | FLSIASAYYM FYDGYEE... .....GSTNA RYGTTVTNDM GTLCFRIVTE |
| hrv-43 | FLSIASAYYM FYDGYEG... .....DQKTS RYGTIASNHM GTLCSRIVTE |
| dq473499-44 | FLSVASAYYM FYDGYNG... .....GDHTA TYGTTVVNRM GTLCVRIVTG |
| hrv-45 | FSSIASAYYM FYDGYDT... .....DGTDA VYGVSVTNDM GTICVRIVTD |
| dq473506-46 | FMSIASAYYM FYDGYES... .....DK.GK IYGTAVTNDM GTICVRIVTE |
| hrv-47 | FLSVASAYYM FYDGYNG... .....DRSGA KYGTTVVNRM GALCMRVVTN |
| dq473496-49 | FLSVASAYYM FYDGYN.... .....EQ.GQ NYGTVSTNNM GSLCSRIVTE |
| hrv-49-f04 | FLSVASAYYM FYDGYN.... ......EEGQ NYGTVSTNNM GSLCSRIVTE |
| hrv-50 | FLSIASAYYM FYDGYDG... .....DKSTS RYGTVVTNDM GTLCSRIVTD |
| hrv-51 | FVSIASAYYM FYDGYEG... .....DSLTS QYGSVVTNAM GTLCVRVVTE |
| dq473507-53 | FMSIASAYYM FYDGYE.... .....DDNGT TYGAVVTNDM GTLCVRIVTE |
| hrv-54 | FLSIASAYYM FYDGYDG... .....DAPGS RYGTSVTNHM GTLCSRVVTG |
| hrv-54-f05 | FLSIASAYYM FYDGYDG... .....DAPGS RYGTSVTNHM GTLCSRVVTG |
| dq473511-55 | FLSIASAYYM FYDGYDG... .....DQTES RYGTAVTNDM GTLCSRIITD |
| hrv-56 | FLSIASAYYM FYDGYDG... .....DTSSS RYGTSVTNHM GTLCSRIVTN |
| hrv-57 | FLSIASAYYM FYDGYDG... .....DQTES RYGTVVTNDM GTLCSRIVTD |
| hrv-58 | FMSIASAYYM FYDGYDG... .....DDAKS IYGSVVTNDM GTICVRIVTS |
| dq473500-59 | FLSIASAYYM FYDGYDG... .....DKSES RYGVSVTNHM GTLCSRIVTN |
| hrv-60 | FLSIASAYYM FYDGYDG... .....HSTES HYGTVVTNDM GTLCSRIVTE |
| hrv-61 | FLSIASAYYM FYDGYDG... .....DSEIT RYGTSVTNDM GALCFRIVTE |
| hrv-62 | FLSVASAYYM FYDGYNG... .....DDYTA KYGTTVVNRM GALCMRIVTN |
| hrv-63 | FLSIASAYYM FYDGYDG... .....DKSSS RYGVSVTNHM GTLCSRIVTN |
| hrv-64 | FLSIASAYYM FYDGYDG... ......GPGS RYGXXVTNDM GTLCSRIVTE |
| ef173417-64 | FLSIASAYYM FYDGYDG... ......GPGS RYGTAVTNDM GTLCSRIVTE |
| hrv-65 | FVSIASAYYM FYDGYEG... .....DDPNS KYGSVVTNAM GTLYVRVVTE |
| hrv-66 | FLGIASAYYM FYDGYDG... .....DTPGS RYGTTVVNHM GTLCIRIVTN |
| hrv-67 | FLSIASAYYM FYDGYDG... ......GPDS LYGTIVTNDM GSLCSRIVTE |
| hrv-68 | FMSIASAYYM FYDGYE.... .....DDKGS VYGSVVTNDM GTLCVRIVTE |
| hrv-71 | FMSIASAYYM FYDGYEG... .....DALSS KYGSVVTNAM GTLCVRVVTE |
| dq473492-73 | FLSIASAYYM FYDGYDG... .....DSAQS HYGTTVVNDM GTLCFRIVTE |
| dq473494-74 | FLSIASAYYM FYDGYDG... .....DSTES HYGTVVTNDM GTLCSRIVTE |
| dq473510-75 | FLSIASAYYM FYDGYEG... .....DQNTS RYGTIASNHM GTLCSRIVTE |
| dq473502-76 | FLSIASAYYM FYDGYDG... .....DQPSS RYGNIVTNDM GTLCSRIVTD |
| hrv-76 | FLSIASAYYM FYDGYDG... .....DQPSS RYGNIVTNDM GTLCSRIVTD |
| hrv-77 | FLSIASAYYM FYDGYDG... .....DTYES RYGTTVVNHM GTLCIRIVTN |
| hrv-78 | FSSIASAYYM FYDGYPD... .....DSTSS PYGTVVTNDM GTLCMRMVTD |
| ef173418-78 | FSSIASAYYM FYDGYSD... .....DSTSS PYGTVVTNDM GTLCMRMVTD |
| hrv-80 | FMSIASAYYM FYDGYES... .....DKGN. IYGTAVTNDM GTLCARIVTE |
| hrv-81 | FLSVASAYYM FYDGYDG... .....DTYHS RYGTVVTXDM GTLCSRIVTS |
| hrv-81-f06 | FLSVASAYYM FYDGYDG... .....DTYQS RYGTVVTNDM GTLCSRIVTS |
| hrv-81-f07 | FLSVASAYYM FYDGYDG... .....DTYQS RYGTVVTNDM GTLCSRIVTS |
| hrv-82 | FLSIASAYYM FYDGYDG... .....DAPGS RYGTIVTNDM GTLCSRIVTE |
| dq473509-82 | FLSIASAYYM FYDGYDG... .....DVPGS RYGTIVTNDM GTLCSRIVTE |
| hrv-85 | FLSVASAYYM FYDGYDG... .....DTPGS MYGTSVTNHM GTLCSRIVTN |
| pico-85 | FLSVASAYYM FYDGYDG... .....DTPGS MYGTSVTNHM GTLCSRIVTN |
| dq473504-88 | FMSIASAYYM FYDGYDG... .....DDASS RYGSVVTNDM GTICIRLVTS |
| a10937-89 | FMSIASAYYM FYDGYDG... .....DSAAS KYGSVVTNDM GTICVRIVTS |
| hrv-89 | FMSIASAYYM FYDGYDG... .....DSAAS KYGSVVTNDM GTICVRIVTS |
| hrv-89-f08 | FMSIASAYYM FYDGYDG... .....DSAAS KYGSVVTNDM GTICVRMVTS |
| hrv-89-f09 | FMSIASAYYM FYDGYDG... .....DNAAS KYGSVVTNDM GTICVRMVTS |
| m16248-89 | FMSIASAYYM FYDGYDG... .....DSAAS KYGSVVTNDM GTICVRIVTS |
| hrv-90 | FLSIASAYYM FYDGYDG... .....DQTDS RYGTIVTNDM GTLCYRIVTD |
| hrv-94 | FLSIASAYYM FYDGYDG... ......GPGS RYGTVVTNDM GTLCSRIVTE |
| ef173419-94 | FLSIASAYYM FYDGYDG... ......GPGS RYGTVVTNDM GTLCSRIVTE |
| hrv-95 | FSSIASAYYM FYDGYDS... .....DGLDA IYGIPVTNHM GTICVRMVTD |
| hrv-96 | FLSIASAYYM FYDGYDG... .....DSEST RYGTSVTNDM GTLCFRIVTE |
| hrv-98 | FLSIASAYYM FYDGYDG... .....DAPGS RYGTSVTNHM GTLCSRVVTG |
| hrv-100 | FLSIASAYYM FYDGYDG... .....DTHDS HYGTTVINHM GTLCMRIVTN |
| ef186077-qpm | FTGLGSAYYM FYDGYDG... ....TDDANI NYGISLTNDM GTLCFRALDG |
| ef077279-nat001 | FTGLGSAYYM FYDGYDG... ....INADNT NYGISETNDM GTLCFRALDG |
| ny1078 | FTGLGSAYYM FYDGYDG... ....TEAGNI QYGISRTNDM GSLCIRALDD |
| ef582385-c024 | FTGLGSAYYM FYDGYDVP.. ....RNKSNA VYGITSTNDM GTLCFRAMED |
| ef077280-nat045 | FTGLGSAYYV FYDGYDE... ....NKVGST TYGISATNDM GTLCFRALED |
| ef582387-c026 | FTGLGSAYYM FYDGYDG... ....TDAANT NYGISLTNDM GTLCFRALDG |
| ef582386-c025 | FTGLGSAYYM FYDGYDK... ....VSHDEG TYGTSVTNDM GRLCFRTPNN |
| dq473485-03 | FVGIASAYNC FYDGYSH... .....DDPDT PYGITVLNHM GSMAFRVVNE |
| ef173422-03 | FVGIASAYNC FYDGYSH... .....DDPDT PYGITVLNHM GSMAFRVVNE |
| dq473490-04 | YLGIASAYNC FYDGYSH... .....DNKTT PYGINVLNHM GSIAFRVVNE |
| hrv-05 | YLGIASAYNC FYDGYSH... .....DNSTT PYGINVLNHM GSMAFRVVNE |
| dq473486-06 | FVGLASAYNC FYDGYSH... .....DNKDT PYGITVLNHM GSIAFRVVNE |
| l05355-14 | YVGLASAYNC FYDGYSH... .....DDAET QYGITVLNHM GSMAFRIVNE |
| k02121-14 | YVGLASAYNC FYDGYSH... .....DDAET QYGITVLNHM GSMAFRIVNE |
| ef173420-17 | FIGIASAYSC FYDGYSH... .....DDENT PYGINVLNHM GSIAFRIVND |
| hrv-26 | YLGIASAYNC FYDGYSH... .....DDTKT PYGINVLNHM GSMAFRVVNE |
| hrv-27 | YLGIASAYNN FYDGYSH... .....DSKTT VYGINVLNHM GSIAFRIVNE |
| ef173421-27 | YLGIASAYNN FYDGYSH... .....DSKTT VYGINVLNHM GSIAFRIVNE |
| dq473487-35 | FVGLASAYNC FYDGYSH... .....DDENT PYGITVLNHM GSMAFRIVND |
| hrv-35 | FVGLASAYNC FYDGYSH... .....DDENT PYGITVLNHM GSMAFRIVND |
| ef173423-37 | FVGLASAYNC FYDGYSH... .....DDENT PYGITVLNHM GSMAFRVVDE |
| hrv-42 | YLGIASAYNC FYDGYSH... .....DNLTT PYGINVLNHM GSMAFRVVNE |
| dq473488-48 | FTGIASAYNC FYDGYSH... .....DDENT QYGINVLNHM GSIAFRVVND |
| hrv-52 | FVGIASAYNC FYDGYSH... .....DDANT QYGINVLNHM GSIAFRVVND |
| hrv-52-f10 | FIGIASAYNC FYDGYSH... .....DDPET QYGINVLNHM GSIAFRVVND |
| ef173424-52 | FVGIASAYNC FYDGYSH... .....DDANT QYGINVLNHM GSIAFRVVND |
| hrv-69 | FTGIASAYNC FYDGYSH... .....DNEDT QYGINVLNHM GSIAFRVVND |
| dq473489-70 | FVGIASAYNC FYDGYSH... .....DNEDT PYGISVLNHM GSIAFRIVND |
| hrv-72 | YVGLASAYNC FYDGYSH... .....DDAET QYGISVLNHM GSMAFRIVNE |
| hrv-79 | FVGLASAYNC FYDGYSH... .....DDENT PYGITVLNHM GSMAFRVVND |
| hrv-83 | FVGLASAYNC FYDGYSH... .....DDEDT PYGITVLNHM GSMAFRVVNE |
| hrv-84 | YLGIASAYNN FYDGYSH... .....DSRTT VYGINVLNHM GVMAFRVVNE |
| hrv-86 | FVGLASAYSC FYDGYSH... .....DNEDT PYGITVLNHM GSIAFRVVND |
| hrv-91 | FIGIASAYNC FYDGYSH... .....DNEDT PYGINVLNHM GSIAFRIVND |
| hrv-92 | FVGLASAYNC FYDGYSH... .....DDEDT PYGITVLNHM GSMAFRIVNE |
| ef173425-93 | YLGIASAYNN FYDGYSH... .....DDKTT VYGINVLNHM GSIAFRVVNE |
| hrv-97 | YLGIASAYNN FYDGYSH... .....DNKDT VYGINVLNHM GSIAFRVVNE |
| hrv-99 | YLGIASAYXC FYDGYSH... .....DDPNT PYGINVLNHM GSIAFRVVNE |
| | 851 900 |
| annotation | .....BBBBB BBBBIBB..B BBB....... .......... .......... |
| af499637-cva13 | HNPVRVSSKI RVYMKPKHVR VWCPRPPRAV EYYGPGV... ...DYKANTL |
| af546702-cva21 | SNPHTIHTSV RVYMKPKHIR CWCPRPPRAV LYRGEGV... ...DMISSAI |
| v01149-pv1m | HNPTKVTSKI RVYLKPKHIR VWCPRPPRAV AYYGPGV... ...DYKDGTL |
| d00239-01 | KQEHPVVITT HIYHKAKHTK AWCPRPPRAV PYTHSRVTNY VPKTGDVT.. |
| pico-01 | KQKHSVVITT HIYHKAKHTK AWCPRPPRAV PYTHSHVTNY MPETGDVT.. |
| hrv-01 | KQKHSVVITT HIYHKAKHTK AWCPRPPRAV PYTHSHVTNY MPETGDVT.. |
| x02316-02 | KHIHKVHIMT RIYHKAKHVK AWCPRPPRAL EYTRAHRTNF KIEDRSIQ.. |
| hrv-07 | TQKHNLKIVS RIYHKAKHIK AWCPRPPRAV PYQHTHSTNY VPQNGEVATQ |
| dq473503-07 | TQKHNLKIVS RIYHKAKHIK AWCPRPPRAV PYQHTHSTNY VPQNGEVATQ |
| hrv-08 | KQKIKTKIDS RIYLKAKHIK AWCPRPPRAV TYNHIYNPNY VREGVTPE.. |
| pico-09 | EHGSRVKIST RIYHKAKHVK AWCPRPPRAV EYIHTHVTNY KPSTGDYATV |
| hrv-09 | EHGPRVKIST RIYHKAKHVK AWCPRPPRAV EYIHTHVTNY KPSTGDYATV |
| hrv-09-f01 | EHGSRVKIST RIYHKAKHVK AWCPRPPRAV EYIHTHVTNY KPSEGEYSTV |
| hrv-09-f02 | EHGSRVKIST RIYHKAKHVK AWCPRPPRAV EYIHTHVTNY KPSEGEYSTV |
| dq473498-10 | QQAHEVEITT SVYHKAKHVK AWCPRPPRAV PYTHAHSTNY KPHGKELQI. |
| hrv-10 | QQAHEVEITT SVYHKAKHVK AWCPRPPRAV PYTHAHSTNY KPHGKELQI. |
| ef173414-11 | DHRHKIEVTT RVYHKAKHVK VWCPRPPRAV EYTHTHVTNY KPQEGQVK.. |
| ef173415-12 | QQKHKVKITS RIYHKAKHIS AWGPRPPRAV PYQHIHNPNY KTSNGVPD.. |
| hrv-13 | EHTNKVKVTT RIYHKAKHVK AWCPRPPRAV EYTNVHVTNY KPGTGDVAVS |
| hrv-13-f03 | EHTNKVKVTT RIYHKAKHIK AWCPRPPRAV EYTNVHVTNY KPKTGDVAVS |
| dq473493-15 | EHGTRVEITT RVYHKAKHVK AWCPRPPRAV EYTHTHVTNY KPQDGDVTTV |
| l24917-16 | EQLHKVKVVT RIYHKAKHTK AWCPRPPRAV QYSHTHTTNY KLSSEVHND. |
| hrv-18 | KHKNEVEITT RIYHKAKHVK AWCPRPPRAV EYTHTHVTNY KPKEGREK.. |
| hrv-19 | EHQTQVAITT RVYHKAKHIK AWCPRPPRAV EYTHTHVTNY KPQTGEVTLP |
| hrv-20 | QQTHKVKITS RIFHKAKHIS AWCPRAPRAV PYQHTKSTNL VPRTGEITTH |
| hrv-21 | QHKHKIEVTT RIYHKAKHVK AWCPRPPRAV EYTHTHVTNY KIANHEVT.. |
| hrv-22 | EHQTQVKITT RVYHKAKHVK AWCPRPPRAV GYTHTHVTNY KPSVGDYTLP |
| dq473497-23 | KHIHDMRIMT RVYHKAKHVK AWCPRPPRAL EYTRAHRTNF KIEGENVK.. |
| hrv-24 | KHNHQIEITT RIYHKAKHIK VWCPRPPRAV EYTHTHVTNY KHQTREVK.. |
| ef173416-24 | KHNHQIEITT RIYHKAKHIK VWCPRPPRAV EYTHTHVTNY KHQTREVK.. |
| hrv-25 | KQVHDVEVTT NIYHKAKHVK AWCPRPPRAV PYKYVDFNNY AASDNVDI.. |
| dq473508-28 | QQKHEVKITS IIFHKAKHVS AWCPRPPRAV AYQHTYSPNF VPQEGDVETH |
| hrv-29 | KQAHDVQVTT SIYHKAKHVK AWCPRPPRVV PYKYVGLTNY TLKEEDTV.. |
| dq473512-30 | KHIHDVRIMT RVYHKAKHVK AWCPRPPRAL EYTRAHRTNF KIDGREVK.. |
| hrv-30 | KHIHDVRIMT RVYHKAKHVK AWCPRPPRAL EYTRAHRTNF KIDGREVK.. |
| hrv-31 | KQAHKVEITT NIYHKAKHVK AWCPRPPRAV PYRYVGSTNY KPDENEVTI. |
| hrv-32 | EHGSRVDIST RVYHKAKHVK AWCPRPPRAV EYTHTHVTNY KPSTGDYTTA |
| hrv-33 | NHKHPIEVTT RVYHKAKHVK VWCPRPPRAV EYTHTHVTNY KSTTREVK.. |
| hrv-34 | EHQNRVEITT RVYHKAKHVK TWCPRPPRAV EYTHTHVTNY KVRGKTEK.. |
| dq473501-34 | EHQNRVEITT RVYHKAKHVK TWCPRPPRAV EYTHTHVTNY KVRGKTEK.. |
| dq473505-36 | NQKHDLNIVC RIYHKAKHIK AWCPRPPRAV PYQHTHSTNY IPYKGEIT.. |
| hrv-38 | NHGTQVKITT RIYHKAKHVK AWCPRPPRAV EYRHTHVNNY KPDQGEVTTM |
| dq473495-38 | NHGTQVKITT RIYHKAKHVK AWCPRPPRAV EYRHTHVNNY KPDQGEVTTM |
| ay751783-39 | QQKHLVEVTT RVYHKAKHVK AWCPRAPRAV PYTHSNVTNY KVRDGEPT.. |
| hrv-40 | KQQHEVEITT RIYHKAKHIK AWCPRAPRAV PYTHTHSTNY KPQEGEVQ.. |
| dq473491-41 | EHTNKVKVTT RVYHKAKHVK AWCPRPPRAV EYTNVHVTNY KPKAGAEIVA |
| hrv-43 | EHQNQIEITT RIYHKAKHIK AWCPRPPRAV EYTHTHVTNY KREGKEVET. |
| dq473499-44 | KQAHDVQVTT SIYHKAKHVK AWCPRPPRVV PYKYVGLTNY TLKETDTV.. |
| hrv-45 | QQQHRVKIDS MVYLKAKHIK AWCPRPPRAV TYNHTYNPNY VRADETAT.. |
| dq473506-46 | QQKHKVLITS RIYHKAKHIK AWCPRAPRAV PYQHIYNPNF KTTQPETIPD |
| hrv-47 | KQLHKVEITT NIYHKAKHVK AWCPRPPRAV PYRYVGSTNY KPDQGEVAI. |
| dq473496-49 | KHIHSMHIMT RIYHKAKHVK AWCPRPPRAL EYTRAHRTNF KVEDRDIK.. |
| hrv-49-f04 | KHIHNMHIMT RIYHKAKHVK AWCPRPPRAL EYTRAHRTNF KIQNRDIK.. |
| hrv-50 | EHQNKVEITT RVYHKAKHIK VWCPRPPRAV EYTHTHVTNY KKSDATEK.. |
| hrv-51 | QQKHEVNITS RIYHKAKHVS AWCPRPPRAV AYQHTYSTNF VPKEGFEGL. |
| dq473507-53 | QQKNEVKITS RIYHKAKHIS AWCPRPPRAV AYQHTYSPNF VPQTGTVETH |
| hrv-54 | KQKHPVEITT RVYHKAKHIR AWCPRAPRAV PYTHTRSTNY MPREGDPT.. |
| hrv-54-f05 | KQEHPVEITT RVYHKAKHIR AWCPRAPRAV PYTHTRSTNY MPQDGEPT.. |
| dq473511-55 | NHKHPIEVTT RVYHKAKHIK AWCPRPPRAV EYTHTHVTNY KKTDGTEK.. |
| hrv-56 | KQLHPVEVTT RVYHKAKHIR AWCPRAPRAV PYTHAHVTNY KPQDGDVQ.. |
| hrv-57 | QHTHHVKVTT RVYHKAKHVK AWCPRPPRAI EYTHTHVTNY KVKGKQEETA |
| hrv-58 | KQRHNLNIVC RIYHKAKHIK AWCPRPPRAV PYQFTHSTNY IPDSGEVTTQ |
| dq473500-59 | SQEHPVEVVT RVYHKAKHVK AWCPRAPRAV PYTHSYVTNY KIAGKEPE.. |
| hrv-60 | HHGTQVHVAT RIYHKAKHVK AWCPRPPRAV EYRHTHVNNY RPDDGEAAIT |
| hrv-61 | QHTNQVKITT RIYHKAKHVK VWCPRPPRAV EYTNVHLTNY KPKDSEKQVT |
| hrv-62 | KQVHDVEVTT NIYHKAKHVK AWCPRPPRAV PYKYVDFNNY AASDSVDI.. |
| hrv-63 | SQEHPVEVTT RVYHKAKHIR AWCPRAPRAV PYTHSYVTNY KITGQETE.. |
| hrv-64 | EHTTQVNITT RVYHKAKHVK AWCPRPPRAV GYTHTNVTNY KPSKGEYTPP |
| ef173417-64 | EHTTQVNITT RVYHKAKHVK AWCPRPPRAV GYTHTNVTNY KPSKGEYTPP |
| hrv-65 | RQKHEVNITS RIYHKAKHIS AWCPRPPRAV AYQHTYSTNF VPSGGLTNLR |
| hrv-66 | EQHHNVEITT RVYHKAKHVK AWCPRPLRAV PYTTVNSTNY MPHTGDLQI. |
| hrv-67 | EHGSRVKIAT RVYHKAKHVK AWCPRPPRAV EYIHTHVTNY RPETGEAQTV |
| hrv-68 | QQTHRVKITS RIFHKAKHIS AWCPRAPRAV PYQHTRSTNL VPKEGDIKTH |
| hrv-71 | QQSNTVNITS RIYHKAKHVR AWCPRPPRAV AYQSTYTTNF VPQDGINSIK |
| dq473492-73 | EHTSRVKVTT RIYHKAKHVK AWCPRPPRAV EYTNAHVTNY KPTDGEVTTA |
| dq473494-74 | EHDARVEITT RVYHKAKHVK AWCPRPPRAV EYTHTHVTNY KPQEGDVT.. |
| dq473510-75 | EHRNKVEVTT RIYHKAKHIK AWCPRPPRAV EYTFRRVTNY KRDGQQVEI. |
| dq473502-76 | DHKHKIEVTT RVYHKAKHVK VWCPRPPRAV EYTYTHVTNY KPHSGDVQ.. |
| hrv-76 | DHKHKIEVTT RIYHKAKHVK VWCPRPPRAV EYTYTHVTNY KPHSGDVQ.. |
| hrv-77 | QQNHEVEITT RVYHKAKHIK AWCPRPPRAV PYTAVDSTNY KPMRGDVQI. |
| hrv-78 | QQQHKVTITA RVYHKAKHIS AWGPRPPRAV PYQHIYNPNY KTEEGTPD.. |
| ef173418-78 | QQQHKVTITA RVYHKAKHIS AWGPRPPRAV PYQHIYNPNY KTEEGTPD.. |
| hrv-80 | QQKHKVLITS RIYHKAKHIK AWCPRAPRAV PYQHTYSPNF KNTDESIPDT |
| hrv-81 | EQVHKVKIVT RIYHKAKHTK AWCPRPPRAV QYTHTHVTNY KLETDVHTS. |
| hrv-81-f06 | EQVHKVKVVT RIYHKAKHTK AWCPRPPRAV QYTHTHVTNY KLETDVHTS. |
| hrv-81-f07 | EQVHKVKVVT RIYHKAKHTK AWCPRPPRAV QYTHTHVTNY KLETDVHTS. |
| hrv-82 | EHQTQVSITT RIYHKAKHVK AWCPRPPRAV GYTHTHVTNY KPSQGDYSVV |
| dq473509-82 | EHQTQVNITT RIYHKAKHVK AWCPRPPRAV GYTHTHVTNY KPSEGDYSVS |
| hrv-85 | KQQHEVEITT RVYHKAKHVK AWCPRAPRAV PYTHTRSTNY VPQDGEVK.. |
| pico-85 | KQQHEVEITT RVYHKAKHVK AWCPRAPRAV PYTHTRSTNY VPQDGEVK.. |
| dq473504-88 | TQKHKLNIIS RIYHKAKHIK AWCPRPPRAV PYQHTHSTNY VPKDGEVATQ |
| a10937-89 | NQKHDSNIVC RIYHKAKHIK AWCPRPPRAV AYQHTHSTNY IPSNGEATTQ |
| hrv-89 | NQKHDLNIVC RIYHKAKHIK AWCPRPPRAV AYQHTHSTNY IPSNGEATTQ |
| hrv-89-f08 | NQKHDLNIVC RIYHKAKHIK AWCPRPPRAV AYQHTHSTNY IPLNGEATTQ |
| hrv-89-f09 | NQKHDLNIVC RIYHKAKHIK AWCPRPPRAV AYQHTHSTNY IPLNGEATTQ |
| m16248-89 | NQKHDSNIVC RIYHKAKHIK AWCPRPPRAV AYQHTHSTNY IPSNGEATTQ |
| hrv-90 | EHAHKIEITT RIYHKAKHIK VWCPRPPRAV EYTHTHVTNY KHATRELK.. |
| hrv-94 | EHKTQVNITT RVYHKAKHVK AWCPRPPRAV GYTHTHVTNY KPSEGEYKPP |
| ef173419-94 | EHKTQVNITT RVYHKAKHVK AWCPRPPRAV GYTHTHVTNY KPSEGEYKPP |
| hrv-95 | KQKIKTKIDS RIYLKAKHIK AWCPRPPRAV TYNHIYNPNY VREGVTPE.. |
| hrv-96 | EHTNKVKITT RVYHKAKHVK VWCPRPPRAV EYTNVHLTNY KPNNEVTTF. |
| hrv-98 | KQEHPVEITT RVYHKAKHIR AWCPRAPRAV PYTHTRSTNY MPQDGEPT.. |
| hrv-100 | QQAHEVEITT NIYHKAKHVK AWCPRPPRAV PYTHSHSTNY KPHEGDVKI. |
| ef186077-qpm | TGASD....I KVFGKPKHIT AWIPRPPRAT QYLHKFSTNY NKPKTSGSTE |
| ef077279-nat001 | AENSE....I KIFAKPKHTT AWIPRPPRAT QYLHKFSTNY NKQKPDSGGQ |
| ny1078 | TTKND....V KVFAKPKHTT AWIPRPPRAT QYTLKYSTNY NVIKKGTTSD |
| ef582385-c024 | TNEHS....I RVFVKPKHTI AWIPRPPRAT QYTHKFSTNY HVKKPDDTTG |
| ef077280-nat045 | TAKQD....V KVFVKPKHIT AWCPRPPRAV DYTHRYSPNY HTNK.GSTTE |
| ef582387-c026 | TEVSD....I KVFAKPKHTA AWIPRPPRAT QYLHKFSTNY NKPKTKGSNE |
| ef582386-c025 | NSGTDI...I RIFGKPKHTR AWIPRPPRAT EYTHKDSTNY NRRVNPNSED |
| dq473485-03 | HDVHTTIVKI RVYHRAKHVE AWIPRAPRAL PYVSIGRTNY PRDSKT.... |
| ef173422-03 | HDVHTTIVKI RVYHRAKHVE AWIPRAPRAL PYVSIGRTNY PRDSKT.... |
| dq473490-04 | HDKHTTHVKI RVYHRAKHIR AWIPRAPRAL QYLYIGRTNY SPNPVS.... |
| hrv-05 | HDNHTTHVKV RVYHRAKHIR AWVPRAPRAL EYLHIGRTNY KQSPQN.... |
| dq473486-06 | HDAHKTLVKI RVYHRAKHVE AWIPRAPRAL PYETIGRTNY PKKNKIV... |
| l05355-14 | HDEHKTLVKI RVYHRAKHVE AWIPRAPRAL PYTSIGRTNY PKNTE..... |
| k02121-14 | HDEHKTLVKI RVYHRAKLVE AWIPRAPRAL PYTSIGRTNY PKNTE..... |
| ef173420-17 | HDVHTTLVKI RVFHRAKHIR AWVPRPPRAL PYTSIGRTNF PNTRNK.... |
| hrv-26 | HDSHTTHVKI RVYHRAKHVR AWVPRAPRAL QYLHIGRTNY DPQSKN.... |
| hrv-27 | NDVHLTKVKI RVYHRAKHVL AWIPRPPRAK PYLSIGRTNY EKTGN..... |
| ef173421-27 | NDVHLTKVKI RVYHRAKHVL AWIPRPPRAK PYLSIGRTNY EKTGN..... |
| dq473487-35 | HDVHTTLVKI RVYHRAKHVQ AWIPRAPRAL PYVSIGRSNY DKSAK..... |
| hrv-35 | HDVHTTLVKI RVYHRAKHVQ AWIPRAPRAL PYVSIGRSNY DKSAK..... |
| ef173423-37 | HDAHTTLVKI RVYHRAKHVE AWIPRAPRAL PYEAIGKTNY PKMIT..... |
| hrv-42 | HDIHTTHVKI RVYHRAKHIR AWIPRAPRVL EYLHIGRTNY KQNPKN.... |
| dq473488-48 | HDSHTTLVKI RVFHRAKHIR AWLPRPPRAL PYTSIGRTNF PTQHGK.... |
| hrv-52 | HDLHKTLVKI RVFHRAKHIR AWVPRPPRAL PYTSIGRTNF PLNHGK.... |
| hrv-52-f10 | HDFHKTLVKI RVFHRAKHIR AWVPRPPRAL PYTSIGRTNF PLSHGK.... |
| ef173424-52 | HDLHKTLVKI RVFHRAKHIR AWVPRPPRAL PYTSIGRTNF PLNHGK.... |
| hrv-69 | HDEHTTIVKI RVFHRAKHIR AWIPRPPRAL PYTSIGRTNF PNTHGK.... |
| dq473489-70 | HDVHTTLVKI RVFHRAKHIR AWVPRPPRAL PYTSIGKTNF PNTHSK.... |
| hrv-72 | HDTHRTLVKI RVYHRAKHIE AWVPRAPRAL PYTSIGRTNY PKNPK..... |
| hrv-79 | HDTHTTEVKI RVYHRAKHVE AWIPRAPRAL PYMSIGRTNY QRDAK..... |
| hrv-83 | HDAHTTEVKI RVYHRAKHVQ VWVPRAPRAL PYVSIGRTNY ERQNIK.... |
| hrv-84 | NDQHXTKVKI RIYHRAKHVM AWIPRPPRAK PYLHIGRTNY DGSQS..... |
| hrv-86 | HDLHKTVVKI RVYHRAKHIQ TWIPRAPRAL PYETIGRTNF PRNPPK.... |
| hrv-91 | HDVHTTLVKI RVFHRPKHIR AWIPRPPRAL PYTSIGRTNF PNRHSK.... |
| hrv-92 | HDAHTTEVKI RVYHRAKHVE AWIPRAPRAL PYVSIGRTNY NKQAIV.... |
| ef173425-93 | NDAHLTKVKI RVYHRAKHVM AWIPRPPRAK PYLKIGRTNY DKSGH..... |
| hrv-97 | NDVHLTKVKI RVYHRAKHVM AWIPRPPRAK PYLKIGRTNY TGSDN..... |
| hrv-99 | HDTHTTYVKI RVYHRAKHIR AWIPRAPRAL QYLHIGRTNY DLNSTN.... |
| | 901 950 |
| annotation | .......... ........VG P....HHH.. BBBBB..HHH .....HHH.. |
| af499637-cva13 | ......TPLP IK..NLTTYG FGHQLKAVYV AGYKIVNYHL ATSEDYRNCV |
| af546702-cva21 | ......LPLT .KVDSITTFG FGHQNKAVYV AGYKICNYHL ATPSDHLNAI |
| v01149-pv1m | ......TPLS TKD..LTTYG FGHQNKAVYT AGYKICNYHL ATQDDLQNAV |
| d00239-01 | .......TAI VPRASMKTVG PSDLYVHVGN LIYRN..LHL FNSE.MHDSI |
| pico-01 | .......TAI VRRNTITTAG PSDLYVHVGN LIYRN..LHL FNSE.MHDSI |
| hrv-01 | .......TAI VRRNTITTAG PSDLYVHVGN LIYRN..LHL FNSE.MHDSI |
| x02316-02 | .......TAI VTRPIITTAG PSDMYVHVGN LIYRN..LHL FNSE.MHESI |
| hrv-07 | ......IKTR ANLFTLKSAG PSSMYVHVGN LIYRN..LHL FNSE.LHDSI |
| dq473503-07 | ......IKTR ANLFTLKSAG PSSMYVHVGN LIYRN..LHL FNSE.LHDSI |
| hrv-08 | .......TKV KYRAEVTTIG PSEMFVHTTN LMYRN..YHL TPEQELDSAI |
| pico-09 | ......IPVR DNVRAVKNVG PSDMYVHVGN LIYRN..LHL FNSE.ANNSV |
| hrv-09 | ......IPVR DNVRAVKNVG PSDMYVHVGN LIYRN..LHL FNSE.ANDSV |
| hrv-09-f01 | ......IPVR DNVRAVRNVG PSDMYVHVGN LIYRN..LHL FNSE.ANDSV |
| hrv-09-f02 | ......IPVR DNVRAVRNVG PSDMYVHVGN LIYRN..LHL FNSE.ANDSV |
| dq473498-10 | ......FIRS RDDPKVVTAG PSDMYVHVGN LIYRN..LHL FNSE.MHDSI |
| hrv-10 | ......FIRS RDDPKVVTAG PSDMYVHVGN LIYRN..LHL FNSE.MHDSI |
| ef173414-11 | .......TAV KARKTIKTAG PSDMYVHVGN LIYRN..LHL FNSD.INDSI |
| ef173415-12 | .......NRV KLRETLTTVG PSDLYVHCGD LIYRN..LHL CEPEHLNDSV |
| hrv-13 | ......IVPR ANVREIRNFG PSDMYVHVGN LIYRN..LHL FNSE.MHDSI |
| hrv-13-f03 | ......IVPR TNVREIRNFG PSDMYVHVGN LIYRN..LHL FNSE.MHDSI |
| dq473493-15 | ......IPTR ENVRAIVNVG PSDMYVHVGN LIYRN..LHL FNSE.ANDSV |
| l24917-16 | .......VAI RPRTNLTTVG PSDMYVHVGN LIYRN..LHL FNSD.IHDSI |
| hrv-18 | .......TAI VPRARITMAG PSDMYVHVGN LIYRN..LHL FNSE.MHDSI |
| hrv-19 | ......IEIR DNPRHIKNVG PSDMYVHVGN LIYRN..LHL FNSD.MHDSI |
| hrv-20 | IRFRNTVKDL TYPTEMTNVG PSDLYVHAGN LIYRN..LHL CEPENLNDST |
| hrv-21 | .......SAV ESRRTIVTVG PSDMYVHVGN LIYRN..LHL FNSE.MHDSI |
| hrv-22 | ......IPTR ANPRQILNVG PSDMYVHVGN LIYRN..LHL FNSD.IHDSV |
| dq473497-23 | .......SRV AHRPAVITAG PSDMYVHVGN LIYRN..LHL FNSE.MHESI |
| hrv-24 | .......TAI EPRRGIKTVG PSDMYVHVGN LIYRN..LHL FNSE.MHDSI |
| ef173416-24 | .......TAI EPRRGIKTVG PSDMYVHVGN LIYRN..LHL FNSE.MHDSI |
| hrv-25 | ........FI QPRNSLKTAG PSDMYVHVGN LIYRN..LHL FNSE.MHDSI |
| dq473508-28 | ......IKFR TDVKQITTVG PSDLYVHTGN LVYRN..LHL CEPENLNDST |
| hrv-29 | .........V ESRPSLMTAG PSDMYVHVGD LIYRN..LHL CNSE.MHDSI |
| dq473512-30 | .......SRV EHRARVTTAG PSDMYVHVGN LIYRN..LHL FNSE.MHDSI |
| hrv-30 | .......SRV EHRARVTTAG PSDMYVHVGN LIYRN..LHL FNSE.MHDSI |
| hrv-31 | ......FVKH RDNPKIITAG PSDLFVHVGN LIYRN..LHL FNSE.MHDSV |
| hrv-32 | ......IQTR EHVRAVKNVG PSDMYVHVGN LIYRN..LHL FNSE.ASDSV |
| hrv-33 | .......TAI RPRATIKTAG PSDMYVHVGN LIYRN..LHL FNSD.IHDSI |
| hrv-34 | .......TAI KHRAKITMAG PSDMYVHVGN LIYRN..LHL FNSE.MHDSI |
| dq473501-34 | .......TAI KHRAKITMAG PSDMYVHVGN LIYRN..LHL FNSE.MHDSI |
| dq473505-36 | .......TQI KTRPNVFTVG PSSMFVHVGN LIYRN..LHL FNSA.LDDSI |
| hrv-38 | ......IPTR TNIRTIVNVG PSDMYVHVGN LIYRN..LHL FNSE.VSDSV |
| dq473495-38 | ......IPTR TNIRTIVNVG PSDMYVHVGN LIYRN..LHL FNSE.VSDSV |
| ay751783-39 | .......LFI KPRENLTTAG PSDMYVHVGN LIYRN..LHL FNSE.MHDSI |
| hrv-40 | .......IFL KERASLTTVG PSDMYVHVGN LIYRN..LHL FNSE.MHDSI |
| dq473491-41 | .....SVRPR DNVRQVRNYG PSDMYVHVGN LIYRN..LHL FNSE.MHDSI |
| hrv-43 | ......AIVS RRDIKIVNAG PTDMYVHVGN LIYRN..LHL FNSE.MHESV |
| dq473499-44 | .........V EPRHSIMTAG PSDMYVHAGN LIYRN..LHL CNSE.MHDSI |
| hrv-45 | ........KV QTRANVTTVG PSDMFIHASE FLYRN..YHL TPEKELKEAC |
| dq473506-46 | T....HIKIR RDIKYIKTAG PSDPHVHAGD LIYRN..LHL CESENLNDSI |
| hrv-47 | ......FIEH RENPKFITAG PSDLFVHVGN LIYRN..LHL FNSE.AHDSV |
| dq473496-49 | .......TGI TSRAIITTAG PSDMYVHVGN LIYRN..LHL FNSE.MHESI |
| hrv-49-f04 | .......TGI KSRPIITTAG PSDMYVHVGN LIYRN..LHL FNSE.MHESI |
| hrv-50 | .......TAI ATRPKITVAG PSDMYVHVGN LIYRN..LHL FNSE.MHDSI |
| hrv-51 | ....KTQIKT RADIKLVNFG PSDLFVHMDD LVYRN..LHL CEPENLNDSV |
| dq473507-53 | I...KFRPDV KDVTSVMTAG PSDLYVHSSN FIYKN..LHL CEPENLNDSV |
| hrv-54 | .......IFL KHRTNLVTAG PSDMYVHVGN LIYRN..LHL FNSE.MHDSI |
| hrv-54-f05 | .......IFL KHRETLTTAG PSDMYVHVGN LIYRN..LHL FNSE.MHDSI |
| dq473511-55 | .......TAI KYRRDIKTVG PSDMYVHVGN LIYRN..LHL FNSD.MHDSI |
| hrv-56 | .......IFL KPRPSLTTFG PSDMYVHVGN LIYRN..LHL FNSD.MHDSI |
| hrv-57 | ......IKYR RDIKVVKNVG PSDMYVHVGN LIYRN..LHL FNSD.MHDSI |
| hrv-58 | ......IKPR TNVFTITSAG PSSMFVHVGN LIYRN..LHL FNSD.LDDSI |
| dq473500-59 | .......IFL KPRMNITTAG PSDMYVHVDN LIYRN..LHL FNSE.MHESV |
| hrv-60 | ......IPIR TDIRAIRTVG PSDMYVHVGN LIYRN..LHL FNSE.ISDSV |
| hrv-61 | T....FIKPR ANLREIRTFG PSDMYVHVGN LIYRN..LHL FNSE.AHDSV |
| hrv-62 | ........FI KSRQNLQTAG PSDMYVHVGN LIYRN..LHL FNSE.MHDTI |
| hrv-63 | .......IFL KPRATIKTAG PSDMYVHVGN LIYRN..LHL FNSE.MHESI |
| hrv-64 | ......VPSR NSPRDIVTVG PSDMYVHVGN LIYRN..LHL CNSD.MHDSI |
| ef173417-64 | ......VPSR NSPRDIVTVG PSDMYVHVGN LIYRN..LHL CNSD.MHDSI |
| hrv-65 | .....TQIKT RDNIKIVNFG PSDMFVHMDD LIYRN..LHL CEPENLNDSV |
| hrv-66 | ......FIKP RTDPKVVNVG PSDMFVHAGN LIYRN..LHL FNSE.MHDSV |
| hrv-67 | ......IPVR ADVRTIRNVG PSDLYVHVGN LIYRN..LHL FNSE.ANDSV |
| hrv-68 | IKFRNTVKDL AYPPELANLG PSDLYVHTGN LIYRN..LHL CEPENLNDST |
| hrv-71 | T....KIKIR RDIKEVSNYG PSDLHVHMGN LVYRN..LHL CDHENLNDSV |
| dq473492-73 | ......IRHR DNVRAIQNFG PSDMYVHVGN LIYRN..LHL FNSE.MHDSI |
| dq473494-74 | .......TVI PTRRSIVNVG PSDMYVHVGN LIYRN..LHL FNSE.ANDSV |
| dq473510-75 | ......AIEP RRDVKFVNAG PTDLYVHVGN LIYRN..LHL LNSE.MHDSI |
| dq473502-76 | .......TAI RPRATIKTAG PSDMYVHVGN LIYRN..LHL FNSD.IHDSI |
| hrv-76 | .......TAI RPRATIKTAG PSDMYVHVGN LIYRN..LHL FNSD.IHDSI |
| hrv-77 | ......FIKE RASPKVVTFG PSDMFVHVGN LVYRN..LHL CNSE.MHDSV |
| hrv-78 | .......TKV AIRANIKTVG PSDLYVHVGN LVYRN..LHL CTNENLSDST |
| ef173418-78 | .......TKV AIRANIKTVG PSDLYVHVGN LVYRN..LHL CTNENLSDST |
| hrv-80 | .....QIKIR DNIRQVRTVG PSDLYVHTGE LIYRN..LHL CEPENLDDSV |
| hrv-81 | .......VAI KPRTSLTNVG PSDMYVHVGN LIYRN..LHL FNSD.MHDSI |
| hrv-81-f06 | .......VAI RPRTSLTNVG PSDMYVHVGN LIYRN..LHL FNSD.MHDSI |
| hrv-81-f07 | .......VAI RPRTSLTNVG PSDMYVHVGN LIYRN..LHL FNSD.MHDSI |
| hrv-82 | ......IPVR ESPRQILNVG PSDMYVHVGN LIYRN..LHL FNSD.AHDSI |
| dq473509-82 | ......IPIR DSPRHILNVG PSDMYVHVGN LIYRN..LHL FNSD.VHDSI |
| hrv-85 | ......IFLK E.RASLTTAG PSDMYVHVGN LIYRN..LHL FNSE.MHDSI |
| pico-85 | ......IFLK E.RASLTTAG PSDMYVHVGN LIYRN..LHL FNSE.MHDSI |
| dq473504-88 | ......IKTR RDVYTVTTAG PSSMYVHVGN LIYRN..LHL FNSD.LDESI |
| a10937-89 | ......IKTR PDVFTVTNVG PSSMFVHVGN LIYRN..LHL FNSD.LDDSI |
| hrv-89 | ......IKTR PDVFTVTNVG PSSMFVHVGN LIYRN..LHL FNSD.LDDSI |
| hrv-89-f08 | ......IKTR PDVFTITNVG PSSMFVHVGN LIYRN..LHL FNSD.LDDSI |
| hrv-89-f09 | ......IKTR PDVFTITNVG PSSMFVHVGN LIYRN..LHL FNSD.LDDSI |
| m16248-89 | ......IKTR PDVFTVTNVG PSSMFVHVGN LIYRN..LHL FNSD.LDDSI |
| hrv-90 | .......TAI RPRKTITTAG PSDMYVHVGN LIYRN..LHL FNSE.MYDSV |
| hrv-94 | ......VPVR NSPRDIVTVG PSDMYVHVGN LIYRN..LHL FNSE.MHDSI |
| ef173419-94 | ......VPVR NSPRDIVTVG PSDMYVHVGN LIYRN..LHL FNSE.MHDSI |
| hrv-95 | .......TKV KYRAEVTTIG PSEMFVHTTN LMYRN..YHL TPEQELDSAI |
| hrv-96 | ......IKPR ENLRDIRNFG PSDMYVHVGN LIYRN..LHL FNSE.ISDSV |
| hrv-98 | .......IFL KHRKDLVTAG PSDMYVHVGN LIYRN..LHL FNSE.MHDSI |
| hrv-100 | ......FIRP RDDPKFVTAG PSDMYVHVGN LIYRN..LHL FNSE.MHDSI |
| ef186077-qpm | LEPKHFFKYR QDITSITNLG PSDMYVHTKE AIYKC..AHL ..TEPNENTI |
| ef077279-nat001 | VEPKHFLNTR N...DIKNLG PSDMFVHTKE AIYKC..AHL ..TKPNEQTI |
| ny1078 | LEQKHFLTYR TDIT...NVG PSDMFVHTKE AVYKC..AHL ..TTPSEKTI |
| ef582385-c024 | LL..IQKHFI NHRTDIKTAG PSDLFVHTAT MIYRN..AHL ..TSLNDKTV |
| ef077280-nat045 | LEEKHYIN.. .TRTTIKTAG PSDLFVHTEQ AIYKN..AHL ..TTPNDQTI |
| ef582387-c026 | LEPKHFLKYR DDITSITTLG PSDMYVHTKE AIYRC..AHL ..TEPNENTI |
| ef582386-c025 | ST.LTRDHYI KTRATVTTAG PSDMYVHTKE AIYRN..AHL ..TSANEQTI |
| dq473485-03 | ........II KKRTNIKTYG LGPRFGGVFT SNVKIINYHL MTPDDHLNLV |
| ef173422-03 | ........II KKRTNIKTYG LGPRFGGVFT SNVKIINYHL MTPDDHLNLV |
| dq473490-04 | ......PI.. KTRESINTYG LGPRFGGVHT GNIKIMNYHL MTHEDNLRLL |
| hrv-05 | ......PI.. KTRKTISTYG LGPRFGGVCT GNIKIMNYHL MTHEDNLNLV |
| dq473486-06 | ......PVI. KKRENITTYG LGSRYGGVFT SNVKIMNYHL MTPEDHLNLI |
| l05355-14 | ......PVIK KRKGDIKSYG LGPRYGGIYT SNVKIMNYHL MTPEDHHNLI |
| k02121-14 | ......PVIK KRKGDIKSYG LGPRYGGIYT SNVKIMNYHL MTPEDHHNLI |
| ef173420-17 | ........VI ARRDSITTYG GNLKFGAVYT CNYKIMNYHL MTHEDHLNLL |
| hrv-26 | ......PI.. KTRXTISTYG LGPRFGGIHT GNVKIMNYHL MTHEDNLNLI |
| hrv-27 | ........II PKRNNISTYG LGPRFGGVCT GNIKIMNYHL MTHEDTLNLQ |
| ef173421-27 | ........II PKRNNISTYG LGPRFGGVCT GNIKIMNYHL MTHEDTLNLQ |
| dq473487-35 | ......PVI. KRREQITKYG LGPRFGGVFT SNFKIMNYHL MTPEDHLNLI |
| hrv-35 | ......PVI. KRREQITKYG LGPRFGGVFT SNFKIMNYHL MTPEDHLNLI |
| ef173423-37 | ......PVI. KKRDNITTYG LGSRYGGVFT SNVKIMNYHL MTPEDHLNLI |
| hrv-42 | ......PI.. KTRDSISTYG LGPRFGGVCT GNIKIMNYHL MSHEDTLNLV |
| dq473488-48 | ........VI AKRESLHTYG SNPRFGAIYT CNYKIMNYHL MTQEDHLNLL |
| hrv-52 | ........II AKRDKITTYG GNSRFGAVYT CNYKIMNYHL MTQEDHLNLL |
| hrv-52-f10 | ........II AKREKITTYG GSPRFGAIYT CNYKIMNYHL MTQEDHLNLL |
| ef173424-52 | ........II AKRDKITTYG GNSRFGAVYT CNYKIMNYHL MTQEDHLNLL |
| hrv-69 | ........VI TRRNKITTYG GSPRFGAVYT CNYKIMNYHL MTHEDHLNLL |
| dq473489-70 | ........VI ATRDSITTYG GNSRFGAIYT CNYKIMNYHL MTHEDYLNLL |
| hrv-72 | ......PVIK KREGDIKTYG LGPRFGGVYT SNAKIMNFHL MTPEDHLNLV |
| hrv-79 | ......PII. AKRENIMKYG LGPRFGGVFT SNVKIMNYHL MTPEDHLNLV |
| hrv-83 | ......PVI. EKRTSIKQYG LGPRFGGVYT GNVKIMNYHL MTPEDQLNLV |
| hrv-84 | ........II PKRDTISTYG LGPRFGGVCT GSVKVMNYHL MTQEDSLNLQ |
| hrv-86 | ........II KKRDTINTYG LGPRFGGIFT GNVKIMNYHL MTPEDHLNLV |
| hrv-91 | ........VI ATRDSITTYG GNSRFGAVYT CNYKIMNYHL MTHEDHLNLL |
| hrv-92 | ......PVI. KKRSLITNYG IGPRFGGVFT GNVKIMNYHL MTPEDHLNLV |
| ef173425-93 | ........VI PKRDGISTYG FGPRFGGVCT GNIKIMNYHL MTHEDMLNLQ |
| hrv-97 | ........II PKRKGISTYG FGPRFGGVCT GSIKIMNYHL MTHEDTLNLQ |
| hrv-99 | ......PI.. KTRDSISTYG LGPRFGGVCT GNVKIMNYHL MTHEDNLNLI |
| | 951 1000 |
| annotation | BBBHHHDBBB BB........ .......BBB BBBHHH.BBB BBB.BBB.BB |
| af499637-cva13 | RSMWERDLMV VESKAQGSDQ IARCSCKTGV YYCESRNKHY PITITAPTFQ |
| af546702-cva21 | SMLWDRDLMV VESRAQGTDT IARCSCRCGV YYCESRRKYY PVTFTGPTFR |
| v01149-pv1m | NVMWSRDLLV TESRAQGTDS IARCNCNAGV YYCESRRKYY PVSFVGPTFQ |
| d00239-01 | LVSYSSDLII YRTNTTGDDY IPSCNCTEAT YYCKHKNRYY PIKVTPHDWY |
| pico-01 | LVSYSSDLII YRTNTIGDDY IPNCNCTEAT YYCRHKNRYY PIKVTPHDWY |
| hrv-01 | LISYSSDLII YRTNTIGDDY IPNCNCTEAT YYCRHKNRYY PIKVTPHDWY |
| x02316-02 | LVSYSSDLII YRTNTVGDDY IPSCDCTQAT YYCKHKNRYF PITVTSHDWY |
| hrv-07 | LVSYSSDLII YRTNTEGDDV IPNCNCTDCT YYCYHKDRYF PIKVTAHDWY |
| dq473503-07 | LVSYSSDLII YRTNTEGDDV IPNCNCTDCT YYCYHKDRYF PIKVTAHDWY |
| hrv-08 | QVVYTADLVI HRTNDKGDDY IPDCNCTDCC YYCAHKNRYI PVKVRYYNYY |
| pico-09 | LISYSSDLVI YRTNTIGDDY IPTCDCTDAT YYCKHKNRYY PIKVTSHDWY |
| hrv-09 | LISYSSDLVI YRTNTIGDDY IPTCDCTDAT YYCKHKNRYY PIKVTSHDWY |
| hrv-09-f01 | LISYSSDLVI YRTNTTGDDY IPTCDCTDAT YYCKHKNRYY PIKVTSHDWY |
| hrv-09-f02 | LISYSSDLVI YRTNTTGDDY IPTCDCTDAT YYCKHKNRYY PIKVTSHDWY |
| dq473498-10 | LVSYSSDLVI YRTNTIGNDY IPTCDCTDAT YYCKHKDRYY PIKVTSHDWY |
| hrv-10 | LVSYSSDLVI YRTNTIGNDY IPTCDCTDAT YYCKHKDRYY PIKVTSHDWY |
| ef173414-11 | LVSYSSDLVI YRTNTVGDDY IPTCDCTDAT YFCKHKNRYY PIKVTSHDWY |
| ef173415-12 | LICYSSDLVI YRTNSKGDDY VPSCDCTDCT FYSKHKDRYF PITVKKYNWF |
| hrv-13 | LVSYSSDLVI YRTNTIGDDY IPSCDCTQAT YYCKHKNRYY PIEVTSHDWY |
| hrv-13-f03 | LVSYSSDLVI YRTNTIGDDY IPACDCTQAT YYCKHKNRYY PINVTSHDWY |
| dq473493-15 | LISYSSDLVI YRTNTKGDDY IPTCDCTQAT YYCRHKNRYY PIEVTSHDWY |
| 124917-16 | LVSYSSDLII YRTSTQGDGY IPTCNCTEAT YYCKHKNRYY PINVTPHDWY |
| hrv-18 | LISYSSDLVI YRTNTTGDDY IPTCDCTDAT YYCKHKNRYY PIKVTSHDWY |
| hrv-19 | LVSYSSDLVI YRTNTVGDDY IPTCDCTDAT YYCKHKNRYF PIKVTSHDWY |
| hrv-20 | LIRYSSDLVI YRTNTTGDDM IPTCDCTQAT YYCKHKDRYY PINVRKHQWY |
| hrv-21 | LISYSSDLVI YRTNTTGDDY IPTCDCTEAT YYCKHKNRYF PIKVTSHDWY |
| hrv-22 | LVSYSSDLVI YRTNTTGDDY IPTCDCTDAT YYCKHKNRYY PIKVTSHDWY |
| dq473497-23 | LVSYSSDLII YRTNTIGDDY IPSCDCTQAT YYCKHKNRYF PITVTSHDWY |
| hrv-24 | LISYSSDLVI YRTNTIGDDY IPTCDCTEAT YYCKHKNRYY PIKVTSHDWY |
| ef173416-24 | LISYSSDLVI YRTNTIGDDY IPTCDCTEAT YYCKHKNRYY PIKVTSHDWY |
| hrv-25 | LVSHSSDLVI YRTNTTGNDY IPTCDCTDAT YYCKHKDRYY PIKVTSHDWY |
| dq473508-28 | LICYSSDLVI YRTNTTGNDV IPTCNCTEAT YYCKHKDRYY PINVKKHQWY |
| hrv-29 | LISYSSDLVI YRTNTTGNDY IPTCDCTDAT YYCKHKDRYY PIKVTSHDWY |
| dq473512-30 | LVSYSSDLII YRTNTVGDDY IPSCDCTQAT YYCKHKNRYF PITVTSHDWY |
| hrv-30 | LVSYSSDLII YRTNTVGDDY IPSCDCTQAT YYCKHKNRYF PITVTSHDWY |
| hrv-31 | LVSYSSDLVI YRTNTTGNDY IPTCDCTQAT YYCKHKDRYY PIVVTSHDWY |
| hrv-32 | LISHSSDLVI YRTNTTGDDY IPTCDCTDAT YYCRHKNRYY PIKVTSHDWY |
| hrv-33 | SVSYSSDLVI YRTNTTGDDY IPTCDCTDAT YFCKHKNRYY PIKVTSHDWY |
| hrv-34 | LISYSSDLVI YRTNTIGDDY IPTCDCTEAT YFCKHKNRYF PIRVTSHDWY |
| dq473501-34 | LISYSSDLVI YRTNTIGDDY IPTCDCTEAT YFCKHKNRYF PIRVTSHDWY |
| dq473505-36 | LVSYSSDLVI YRTNTEGDDV IPNCDCTECT YYCHHKDRYF PIKVTPHDWY |
| hrv-38 | LVSYSSDLVI YRTNTTGDDY IPTCDCTDAT YYCKHKNRYF PIKVTSHDWY |
| dq473495-38 | LVSYSSDLVI YRTNTTGDDY IPTCDCTDAT YYCKHKNRYF PIKVTSHDWY |
| ay751783-39 | LVSYSSDLVI YRTNTQGDDY IPTCDCTQAT YYCKHKNRYF PITVTSHDWY |
| hrv-40 | LVSHSSDLVI YRTNTIGDDY IPTCDCTEAT YYCKHKNRYY PIKVTSHDWY |
| dq473491-41 | LVSYSSDLVI YRTNTIGDDY IPSCDCTQAT YYCKHKNRYY PITVTSHDWY |
| hrv-43 | LVSYSSDLVI YRTNTVGDDY IPTCDCTDAT YYCKHKNRYY PIKVTSHDWY |
| dq473499-44 | LISYSSDLVI YRTNTTGNDY IPTCDCTDAT YYCKHKDRYY PIKVTSHDWY |
| hrv-45 | QIVYTADLVI HRTRDKGDDY IPQCNCTDCC YYCAHKDRYI PIKVEYHSYY |
| dq473506-46 | LISYSSDLVI YRTNTTGDDI VPTCDCTLGT YYCKHKDRYY PVSVTKHQWY |
| hrv-47 | LVSYSSDLVI YRTNTTGNDY IPTCDCTQAT YYCRHKDRYY PIDVTSHDWY |
| dq473496-49 | LVSYSSDLII YRTNTIGDDY IPSCDCTQAT YYCKHKNRYF PITVTSHDWY |
| hrv-49-f04 | LVSYSSDLII YRTNTIGDDY IPSCDCTQAT YYCKHKSRYF PITVTSHDWY |
| hrv-50 | LVSYSSDLVI YRTNTIGDDY IPTCDCTDAT YYCKHKNRYY PIKVTSHDWY |
| hrv-51 | LICYSSDLVI YRTNTKGDDV IPTCDCTQAT YYCKHKNRYY PINVKKHDWY |
| dq473507-53 | LISYSSDLVI YRTNTTGDDI IPTCDCTLGT YYCKHKDRYY PISVTKHQWY |
| hrv-54 | LVSYSSDLVI YRTNTIGDDY IPTCDCTEAT YYCKHKNRYY PIKVTSHDWY |
| hrv-54-f05 | LISYSSDLVI YRTNTIGDDY IPTCDCTEAT YYCKHKNRYY PIKVTSHDWY |
| dq473511-55 | LVSYSSDLVI YRTNTIGDDY IPTCDCTEAT YYCKHKNRYY PIKVTSHDWY |
| hrv-56 | LVSYSSDLVI YRTNTTGDDY IPTCDCTQAT YYCKHKNRYF PINVNSHDWY |
| hrv-57 | LVSYSSDLVI YRTNTTGDDY IPTCDCTEAT YYCRHKNRYY PIKVTSHDWY |
| hrv-58 | LVSYSSDLII HRTNTEGDDI IPNCDCTECT YYCHHRDRYF PIKVTAHDWY |
| dq473500-59 | LVSYSSDLVI YRTNTKGDDY IPTCDCTQAT YYCKHKNRYY PINVTSHDWY |
| hrv-60 | LVSYSSDLVI YRTNTTGDDY IPTCDCTDAT YYCKHKNRYY PIKVTSHDWY |
| hrv-61 | LVSYSSDLVI YRTNTVGDDF IPTCDCTQAT YYCKHKNRYF PITVTSHDWY |
| hrv-62 | LVSXSSDLVI YRTNTTGDDY IPTCDCTDAT YYCKHKNRYY PIKVTSHDWY |
| hrv-63 | LISYSSDLVI YRTNTKGDDY IPTCDCTQAT YYCKHKNRYY PINVTSHDWY |
| hrv-64 | LVSYSSDLVI YRTNTIGDDY IPMCDCTDAT YYCKHKNRYY PIKVTSHDWY |
| ef173417-64 | LVSYSSDLVI YRTNTIGDDY IPMCDCTDAT YYCKHKNRYY PIKVTSHDWY |
| hrv-65 | SICYSSDLVI YRTNTKGDDL IPTCNCTQAT YYCKHKNRYY PINVKKHDWY |
| hrv-66 | LVSYSSDLVI YRTNTTGNDY IPTCDCTDAT YYCRHKDRYY PIKVTSHDWY |
| hrv-67 | LISYSSDLVI YRTNTVGDDY IPTCDCTEAT YYCRHKNRYY PIRVTSHDWY |
| hrv-68 | LICYSSDLVI YRTNTTGDDI IPTCDCTQAT YYCKHKDRYY PINVRKYQWY |
| hrv-71 | LIRYSSDLVI YRTNTEGDDF IPTCNCTQAT YYCKHKNRYF PINVKKHEWY |
| dq473492-73 | LVSYSSDLVI YRTNTIGDDF IPSCDCTEAT YYCKHKNRYY PIKVTSHDWY |
| dq473494-74 | LISYSSDLVI YRTNTKGDDY IPTCDCTEAT YYCKHKNRYY PIKVTSHDWY |
| dq473510-75 | LISYSSDLII YRTNTKGDDY IPTCDCTEAT YYCRHKNRYY PIKVTSHDWY |
| dq473502-76 | LVSYSSDLVI YRTNTTGDDY IPTCDCTDAT YFCKHKNRYY PIKVTSHDWY |
| hrv-76 | LVSYSSDIVI YRTNTTGDDY IPTCDCTDAT YFCKHKNRYY PIKVTSHDWY |
| hrv-77 | LVSYSSDLVI YRTNTIGNDY IPTCDCTEAT YYCKHKDRYY PIRVTSHDWY |
| hrv-78 | LIRYSSDLVI YRTNTRGDDY IPTCDCTQCT YYCKSKGRYY PIIVRKHDWY |
| ef173418-78 | LIRYSSDLVI YRTNTRGDDY IPTCDCTQCT YYCKSKGRYY PIIVRKHDWY |
| hrv-80 | LICYSSDLVI YRTNTKGDDV IPVCDCTHAT YYCKHKNRYY PIEVKKHQWY |
| hrv-81 | LVSYSSDLVI YRTNTQGDDY IPTCNCTDAT YYCKHKNRYY PIKVTPHDWY |
| hrv-81-f06 | LVSYSSDLVI YRTNTQGDDY IPTCNCTDAT YYCKHKNRYY PIKVTPHDWY |
| hrv-81-f07 | LVSYSSDLVI YRTNTQGDDY IPTCNCTDAT YYCKHKNRYY PIKVTPHDWY |
| hrv-82 | LVSYSSDLVI YRTNTTGDDY IPTCDCTDAT YYCRHKNRYY PIKVTSHDWY |
| dq473509-82 | LVSYSSDLVI YRTNTTGDDY IPTCDCTDAT YYCRHKNRYY PIKVTSHDWY |
| hrv-85 | LVSYSSDLVI YRTNTIGDDY IPTCDCTEAT YYCKHKNRYY PIRVTSHDWY |
| pico-85 | LVSYSSDLVI YRTNTIGDDY IPTCDCTEAT YYCKHKNRYY PIRVTSHDWY |
| dq473504-88 | LVSYSSDLII YRTNTKGDDV IPNCDCTDCT YYCHHKDRYY PIKVTSHDWY |
| a10937-89 | LVSYSSDLII YRTNTEGNDV IPNCDCTECT YYCHHKDRYF PIRVTAHDWY |
| hrv-89 | LVSYSSDLII YRTNTEGNDV IPNCDCTECT YYCHHKDRYF PIRVTAHDWY |
| hrv-89-f08 | LVSYSSDLVI YRTNTEGNDV IPNCDCTECT YYCHHKDRYF PIKVTAHDWY |
| hrv-89-f09 | LVSYSSDLVI YRTNTEGNDV IPNCDCTECT YYCHHKDRYF PIKVTAHDWY |
| m16248-89 | LVSYSSDLII YRTNTEGNDV IPNCDCTECT YYCHHKDRYF PIRVTAHDWY |
| hrv-90 | LVSYSSDLVI YRTNTTGDDY IPTCDCTEAT YYCKHKNRYY PIRVTSHDWY |
| hrv-94 | LISYSSDLVI YRTNTKGDDY IPTCDCTDAT YYCKHKNRYY PIKVTSHDWY |
| ef173419-94 | LISYSSDLVI YRTNTKGDDY IPTCDCTDAT YYCKHKNRYY PIKVTSHDWY |
| hrv-95 | QVVYTADLVI HRTNDKGDDY IPDCNCTDCC YYCAHKNRYI PVKVKYYNYY |
| hrv-96 | LVSYSSDLVI YRTNTIGDDY IPACDCTQAT YYCKHKNRYY PITVKSHDWY |
| hrv-98 | LVSYSSDLVI YRTNTIGDDY IPTCDCTEAT YYCKHKNRYY PIKVTSHDWY |
| hrv-100 | LVSYSSDLVI YRTNTTGNDY IPTCDCTDAT YYCKHKDRYY PIKVTSHDWY |
| ef186077-qpm | LLAISSDLQV DSTSIPGPDN IPTCDCTTGT YYCKSRDRYY PINLTHHSWY |
| ef077279-nat001 | LLAISSDLQV DSVDTPGPDN IPTCDCTTGT YYCKSRDRYF PINLTHHSWY |
| ny1078 | LLAISSDLQI DSADEPGPDI IPTCDCTTGT YFCKSMDRYY PVEFRHHSWY |
| ef582385-c024 | LLALSADLQV DAADQPGPDT IPQCDCLEGC YYSKSLDRYI PVKLEAHDWY |
| ef077280-nat045 | LLALTADLQI DGCDQPGPDN IPDCDCTSGC YYSRSLDRYI PVECEAHDWY |
| ef582387-c026 | LLAISSDLQV DSTNVPGPDN IPTCDCTTGT YYCKSRDRYY PVNLTHHSWY |
| ef582386-c025 | LIALTADLQV DAAEHPGDDI IPDCDCTTGT YYCKSKDRYY PVSVVSHAWY |
| dq473485-03 | APYPNRDLAV VATGAHGAET IPHCNCTSGV YYSRYYRKFY PIICERPTNI |
| ef173422-03 | APYPNRDLAV VATGAHGAET IPHCNCTSGV YYSRYYRKFY PIICERPTNI |
| dq473490-04 | TPMPERDLAI VDARAHGSET VPQCSCTSGV YYSRYYNKYY PVVCEKPTCV |
| hrv-05 | TPMPERDLAI INIGAHGSET VPHCNCTSGV YFSRYYNNFY PVICEKPTCL |
| dq473486-06 | TPYVNRDLAV VFTGAHGAET IPHCNCTSGV YYSRYYRKFY PVICEKPTTI |
| l05355-14 | APYPNRDLAI VSTGGHGAET IPHCNCTSGV YYSTYYRKYY PIICEKPTNI |
| k02121-14 | APYPNRDLAI VSTGGHGAET IPHCNRTSGV YYSTYYRKYY PIICEKPTNI |
| ef173420-17 | TPYYNRDLAV VATGALGSET IPHCNCTSGV YYSQYYNKYY PIICDKPTNI |
| hrv-26 | TPLPERDLAV VGVGAHGSET VPHCNCTSGV YYSRYYNKFY PIVCEKPTCL |
| hrv-27 | TSLPERDLAV ISINAHGSET VPQCSCTGGV YYSRYYNKYY PVVCEKPTCV |
| ef173421-27 | TSLPERDLAV ISINAHGSET VPQCSCTGGV YYSRYYNKYY PVVCEKPTCV |
| dq473487-35 | APYQSRDLAI VATGAHGAET IPHCNCTSGV YYSTYYRKFY PVICEKPTTI |
| hrv-35 | APYQSRDLAI VATGAHGAET IPHCNCTSGV YYSTYYRKFY PVICEKPTTI |
| ef173423-37 | TPYVNRDLAI VYTGAHGAET IPHCNCTSGV YYSRYYRKFY PIMCEKPTTI |
| hrv-42 | TPLPERDLAI VSTGAHGSET VPQCNCMSGV YYSRYYNKFY PVICEKPTCL |
| dq473488-48 | APHYNRDLAV VATGAQGSEI IPHCNCTSGV YYSQYYGKYY PIICEKPTNI |
| hrv-52 | TPYYNRDLAV VATGAQGSEI IPHCNCTSGV YYSQYYRKYY PIICEKPTNI |
| hrv-52-f10 | TPYYNRDLAV VATGAQGSEI IPHCNCTSGV YYSQYYRKYY PIICDKPTNI |
| ef173424-52 | TPYYNRDLAV VATGAQGSEI IPHCNCTSGV YYSQYYRKYY PIICEKPTNI |
| hrv-69 | TPYYDRDLAV VATGAQGSEI VPHCNCTSGV YYSQYYRKYY PIICERPTNX |
| dq473489-70 | TPYYNRDLAV VATGAQGSET IPHCNCTSGV YYSQYYNKYY PIICDKPTNI |
| hrv-72 | TSYPSRDLAI VSTGAHGAET IPHCNCTSGV YYSSYYRKFY PVICEKPTTI |
| hrv-79 | TSYPSRDLAI VFTGAHGAET VPHCNCTSGV YYSTYYRKSY PIICDKPTTI |
| hrv-83 | SSHPNRDLAV VSTGAHGAET IPHCSCTSGV YYSTYYRKFY PVICERSTTI |
| hrv-84 | TPLPERDLAI INLNGHGSET IPHCNCTSGV YYSRHYNKFY PVICEKPTCI |
| hrv-86 | TPYPSRDLAI VSTGAHGAET IPHCSCTSGV YYSRYYNKFY PIICEKPTNI |
| hrv-91 | TPYYNRDLAV VATGAQGSEI IPHCNCTSGV YYSQYYNKYY PIICDKPTNI |
| hrv-92 | APHPNRDLAV VSTGAHGAET IPHCNCTSGV YYSTYYRKFY PIICGKPTTI |
| ef173425-93 | TPLPERDLAV VNINAHGSET VPHCNCTSGV YYSRYYNKYY PIICEKPTCI |
| hrv-97 | TPLPERDLAV VNINAHGSET VPQCNCTSGV YYSRYYNKYY PVICEKPTCI |
| hrv-99 | TPLPERDLAV VSLQGHGSET VPQCKCTSGV YYSRYYNKFY PVICEKPTCL |
| | 1001 1050 |
| annotation | BB........ BBBB.BBBBB .......C.. BBB....BBB BBBBB...BB |
| af499637-cva13 | WMEKNDFYPA RYQSHMTIGN GFAAPGDCGG ILRCQHGVMG LITAGGNGLV |
| af546702-cva21 | FMEANDYYPA RYQSHMLIGC GFAEPGDCGG ILRCTHGVIG IITAGGEGVV |
| v01149-pv1m | YMEANNYYPA RYQSHMLIGH GFASPGDCGG ILRCHHGVIG IITAGGEGLV |
| d00239-01 | EIQESEYYPK HIQYNLLIGE GPCEPGDCGG KLLCRHGVIG IITAGGEGHV |
| pico-01 | EIQESEYYPK HIQYNLLIGE GPCEPGDCGG KLLCRHGVIG IITAGGEGHV |
| hrv-01 | EIQESEYYPK HIQYNLLIGE GPCEPGDCGG KLLCRHGVIG IITAGGEGHV |
| x02316-02 | EIQESEYYPK HIQYNLLIGE GPCEPGDCGG KLLCKHGVIG IVTAGGDNHV |
| hrv-07 | EIQESEYYPK HIQYNILIGE GPCEPGDCGG KLLCKHGVIG IVTAGGEGHV |
| dq473503-07 | EIQESEYYPK HIQYNILIGE GPCEPGDCGG KLLCKHGVIG IVTAGGEGHV |
| hrv-08 | TIQESEYYPK HIQYDILLGE GPSEPGDCGG KLLCKHGVIG MVTAGGDNHV |
| pico-09 | EIQESEYYPK HIQYNLLIGE GPCEPGDCGG KLLCKHGVIG IITAGGEGHV |
| hrv-09 | EIQESEYYPK HIQYNLLIGE GPCEPGDCGG KLLCKHGVIG IITAGGEGHV |
| hrv-09-f01 | EIQESEYYPK HIQYNLLIGE GPCEPGDCGG KLLCKHGVIG MITAGGEGHV |
| hrv-09-f02 | EIQESEYYPK HIQYNLLIGE GPCEPGDCGG KLLCKHGVIG IITAGGEGHV |
| dq473498-10 | EIQESEYYPK HIQYNLLIGE GPCEPGDCGG KLLCKHGVIG IITAGGEGHV |
| hrv-10 | EIQESEYYPK HIQYNLLIGE GPCEPGDCGG KLLCKHGVIG IITAGGEGHV |
| ef173414-11 | EIQESEYYPK HIQYNLLIGE GPCEPGDCGG KLLCKHGVIG IITAGGEGHV |
| ef173415-12 | EIQESEYYPK HIQYNILIGD GPCQAGDCGG KLLCKHGVIG IVTAGGSGKV |
| hrv-13 | EIQESEYYPK HIQYNLLIGE GPCEPGDCGG KLLCKHGVIG IITAGGEGHV |
| hrv-13-f03 | EIQESEYYPK HIQYNLLIGE GPCEPGDCGG KLLCKHGVIG IITAGGEGHV |
| dq473493-15 | EIQESEYYPK HIQYNLLIGE GPCEPGDCGG KLLCKHGVIG IITAGGEGHV |
| 124917-16 | EIQESEYYPK HIQYNLLIGE GPCEPGDCGG KLLCKHGVIG IITAGGEGHV |
| hrv-18 | EIQESEYYPK HIQYNLLIGE GPCEPGDCGG KLLCKHGVIG IITAGGEGHV |
| hrv-19 | EIQESEYYPK HIQYNLLIGE GPCEPGDCGG KLLCKHGVIG IITAGGEGHV |
| hrv-20 | EIQESEYYPK HIQYNILIGE GPCEPGDCGG KLLCKHGVIG IVTAGGDNHV |
| hrv-21 | EIQESEYYPK HIQYNLLIGE GPCEPGDCGG KLLCKHGVIG MITAGGDGHV |
| hrv-22 | EIQESEYYPK HIQYNLLIGE GPCEPGDCGG KLLCKHGVIG IITAGGEGHV |
| dq473497-23 | EIQESEYYPK HIQYNLLIGE GPCEPGDCGG KLLCKHGVIG IVTAGGDNHV |
| hrv-24 | EIQESEYYPK HIQYNLLIGE GPCEPGDCGG KLLCKHGVIG IVTAGGEGHV |
| ef173416-24 | EIQESEYYPK HIQYNLLIGE GPCEPGDCGG KLLCKHGVIG IVTAGGEGHV |
| hrv-25 | EIQESEYYPK HIQYNLLIGE GPCEPGDCGG KLLCKHGVIG IVTAGGEGHV |
| dq473508-28 | EIQESEYYPK HIQYNILIGE GPCEPGDCGG KLLCKHGVIG IVTAGGENHV |
| hrv-29 | EIQESEYYPK HIQYNLLIGE GPCEPGDCGG KLLCKHGVIG IITAGGEGHV |
| dq473512-30 | EIQESEYYPK HIQYNLLIGE GPCEPGDCGG KLLCKHGVIG IVTAGGDNHV |
| hrv-30 | EIQESEYYPK HIQYNLLIGE GPCEPGDCGG KLLCKHGVIG IVTAGGDNHV |
| hrv-31 | EIQESEYYPK HIQYNLLIGE GPCEPGDCGG KLLCKHGVIG IITAGGEGHV |
| hrv-32 | EIQESEYYPK HIQYNLLIGE GPCEPGDCGG KLLCKHGVIG IITAGGEGHV |
| hrv-33 | EIQESEYYPK HIQYNLLIGE GPCEPGDCGG KLLCKHGVIG IITAGGEGHV |
| hrv-34 | EIQESEYYPK HIQYNLLIGE GPCEPGDCGG KLLCKHGVIG IVTAGGEGHV |
| dq473501-34 | EIQESEYYPK HIQYNLLIGE GPCEPGDCGG KLLCKHGVIG IVTAGGEGHV |
| dq473505-36 | EIQESEYYPK HIQYNLLIGE GPCEPGDCGG KLLCKHGVIG MVTAGGDGHV |
| hrv-38 | EIQESEYYPK HIQYNLLIGE GPCEPGDCGG KLLCKHGVIG IITAGGEGHV |
| dq473495-38 | EIQESEYYPK HIQYNLLIGE GPCEPGDCGG KLLCKHGVIG IITAGGEGHV |
| ay751783-39 | EIQESEYYPK HIQYNLLIGE GPCEPGDCGG KLLCKHGVIG IITAGGDNHV |
| hrv-40 | EIQESEYYPK HIQYNLLIGE GPCEPGDCGG KLLCKHGVIG IITAGGEGHV |
| dq473491-41 | EIQESEYYPK HIQYNLLIGE GPCEPGDCGG KLLCKHGVIG IITAGGEGHV |
| hrv-43 | EIQESEYYPK HIQYNLLIGE GPCEPGDCGG KLLCKHGVIG IITAGGEGHV |
| dq473499-44 | EIQESEYYPK HIQYNLLIGE GPCEPGDCGG KLLCKHGVIG IITAGGEGHV |
| hrv-45 | TIQKSDYYPK HIQYDILIGE GPSQPGDCGG KLLCRHGVIG MVTAGGEGHV |
| dq473506-46 | EIQESDYYPK HIQYNILLGV GPCKPGDCGG KLLCKHGVIG IITAGGDNHV |
| hrv-47 | EIQESEYYPK HIQYNLLIGE GPCEPGDCGG KLLCKHGVIG IITAGGEGHV |
| dq473496-49 | EIQESEYYPK HIQYNLLIGE GPCEPGDCGG KLLCKHGVIG IVTAGGDNHV |
| hrv-49-f04 | EIQESEYYPK HIQYNLLIGE GPCEPGDCGG KLLCKHGVIG IVTAGGDNHV |
| hrv-50 | EIQESEYYPK HIQYNLLIGE GPCEPGDCGG KLLCKHGVIG IVTAGGEGHV |
| hrv-51 | EIQESEYYPK HIQCNILIGE GPCEPGDCGG KLLCKHGVIG LLTAGGSGHV |
| dq473507-53 | EIQESEYYPK HIQYNILLGV GPCKPGDCGG KLLCKHGVIG IITAGGDNHV |
| hrv-54 | EIQESEYYPK HIQYNLLIGE GPCEPGDCGG KLLCKHGVIG IITAGGEGHV |
| hrv-54-f05 | EIQESEYYPK HIQYNLLIGE GPCEPGDCGG KLLCKHGVIG IITAGGEGHV |
| dq473511-55 | EIQESEYYPK HIQYNLLIGE GPCEPGDCGG KLLCRHGVIG MVTAGGDGHV |
| hrv-56 | EIQESEYYPK HIQYNLLIGE GPCEPGDCGG KLLCKHGVIG IITAGGEGHV |
| hrv-57 | EIQESEYYPK HIQYNLLIGE GPCEPGDCGG KLLCKHGVIG MITAGGEGHV |
| hrv-58 | EIQESEYYPK HIQYNLLIGE GPCEPGDCGG KLLCKHGVIG IVTAGGDGHV |
| dq473500-59 | EIQESEYYPK HIQYNLLIGE GPCEPGDCGG KLLCKHGVIG IITAGGEGHV |
| hrv-60 | EIQESEYYPK HIQYNLLIGE GPCEPGDCGG KLLCKHGVIG IITAGGEGHV |
| hrv-61 | EIQESEYYPK HIQYNLLIGE GPCEPGDCGG KLLCKHGVIG IITAGGEGHV |
| hrv-62 | EIQESEYYPK HIQYNLLIGE GPCEPGDCGG KLLCKHGVIG IVTAGGEGHV |
| hrv-63 | EIQESEYYPK HIQYNLLIGE GPCEPGDCGG KLLCKHGVIG IITAGGEGHV |
| hrv-64 | EIQESEYYPK HIQYNLLIGE GPCEPGDCGG KLLCKHGVIG IITAGGEGHV |
| ef173417-64 | EIQESEYYPK HIQYNLLIGE GPCEPGDCGG KLLCKHGVIG IITAGGEGHV |
| hrv-65 | EIQESEYYPK HIQYNILIGE GPCKPGDCGG KLLCKHGVIG ILTAGGEGHV |
| hrv-66 | EIQESEYYPK HIQYNLLIGE GPCEPGDCGG KLLCKHGVIG IITAGGEGHV |
| hrv-67 | EIQESEYYPK HIQYNLLIGE GPCEPGDCGG KLLCKHGVIG IITAGGEGHV |
| hrv-68 | EIQESEYYPK HIQYNILMGE GPCEPGDCGG KLLCKHGVIG IVTAGGDNHV |
| hrv-71 | EIQESEYYPK HVQYNILIGE GPCEPGDCGG KLLCSHGVIG IVTAGGEGHV |
| dq473492-73 | EIQESEYYPK HIQYNLLIGE GPCEPGDCGG KLLCKHGVIG IITAGGEGHV |
| dq473494-74 | EIQESEYYPK HIQYNLLIGE GPCEPGDCGG KLLCKHGVIG IVTAGGEGHV |
| dq473510-75 | EIQESEYYPK HIQYNLLIGE GPCEPGDCGG KLLCRHGVIG IVTAGGEGHV |
| dq473502-76 | EIQESEYYPK HIQYNLLIGE GPCEPGDCGG KLLCKHGVIG IITAGGEGHV |
| hrv-76 | EIQESEYYPK HIQYNLLIGE GPCEPGDCGG KLLCKHGVIG IITAGGEGHV |
| hrv-77 | EIQESEYYPK HIQYNLLIGE GPCEPGDCGG KLLCKHGVIG IVTAGGEGHV |
| hrv-78 | EIQESEYYPK HIQYNILIGE GPCEPGDCGG KLLCKHGVIG IVTAGGEGHV |
| ef173418-78 | EIQESEYYPK HIQYNILIGE GPCEPGDCGG KLLCKHGVIG IVTAGGEGHV |
| hrv-80 | EIQESEYYPK HIQYNILIGV GPCEPGDCGG KLLCKHGVIG IVTAGGDDHV |
| hrv-81 | EIQESEYYPK HIQYNLLIGE GPCEPGDCGG KLLCKHGVIG IITAGGEGHV |
| hrv-81-f06 | EIQESEYYPK HIQYNLLIGE GPCEPGDCGG KLLCKHGVIG IITAGGEGHV |
| hrv-81-f07 | EIQESEYYPK HIQYNLLIGE GPCEPGDCGG KLLCKHGVIG IITAGGEGHV |
| hrv-82 | EIQESEYYPK HIQYNLLIGE GPCEPGDCGG KLLCKHGVIG IITAGGEGHV |
| dq473509-82 | EIQESEYYPK HIQYNLLIGE GPCEPGDCGG KLLCKHGVIG IITAGGEGHV |
| hrv-85 | EIQESEYYPK HIQYNLLIGE GPCEPGDCGG KLLCKHGVIG IITAGGEGHV |
| pico-85 | EIQESEYYPK HIQYNLLIGE GPCEPGDCGG KLLCKHGVIG IITAGGEGHV |
| dq473504-88 | EIQESEYYPK HIQYNILIGE GPCEPGDCGG KLLCKHGVIG IVTAGGEGHV |
| a10937-89 | EIQESEYYPK HIQYNLLIGE GPCEPGDCGG KLLCKHGVIG MITAGGEGHV |
| hrv-89 | EIQESEYYPK HIQYNLLIGE GPCEPGDCGG KLLCKHGVIG MITAGGEGHV |
| hrv-89-f08 | EIQESEYYPK HIQYNLLIGE GPCEPGDCGG KLLCKHGVIG MVTAGGEGHV |
| hrv-89-f09 | EIQESEYYPK HIQYNLLIGE GPCEPGDCGG KLLCKHGVIG MVTAGGEGHV |
| m16248-89 | EIQESEYYPK HIQYNLLIGE GPCEPGDCGG KLLCKHGVIG MITAGGEGHV |
| hrv-90 | EIQESEYYPK HIQYNLLIGE GPCEPGDCGG KLLCKHGVIG IITAGGEGHV |
| hrv-94 | EIQESEYYPK HIQYNLLIGE GPCKPGDCGG KLLCKHGVIG IITAGGEGHV |
| ef173419-94 | EIQESEYYPK HIQYNLLIGE GPCKPGDCGG KLLCKHGVIG IITAGGEGHV |
| hrv-95 | TIQESEYYPK HIQYDILLGE GPSEPGDCGG KLLCKHGVIG MVTAGGDNHV |
| hrv-96 | EIQESEYYPK HIQYNLLIGE GPCEPGDCGG KLLCKHGVIG IITAGGEGHV |
| hrv-98 | EIQESEYYPK HIQYNLLIGE GPCEPGDCGG KLLCKHGVIG IITAGGEGHV |
| hrv-100 | EIQESEYYPK HIQYNLLIGE GPCEPGDCGG KLLCKHGVIG IITAGGEGHV |
| ef186077-qpm | IIQESSYYPK HIQYDILIGE GPCSPGDCGG KLLCTHGTIG MVTAGGENHV |
| ef077279-nat001 | IIQESDYYPK HIQYDILIGE GPCSPGDCGG KLLCVHGTIG MVTAGGENHV |
| ny1078 | EIQESIYYPK HIQYDILIGE GPCSPGDCGG KLLCVHGTIG MITAGGENHV |
| ef582385-c024 | QIEETCYYPQ HIQYNIMIGE GHCQPGDCGG KLLCTHGVIG IITAGGDNHV |
| ef077280-nat045 | PVEETQYYPK HIQYNLLIGE GPCVPGDCGG KLLCRHGVIG IITAGGDGHV |
| ef582387-c026 | IIQESSYYPK HIQYDILIGE GPCSPGDCGG KLLCIHGTIG MVTAGGENHV |
| ef582386-c025 | PIEESSYYPK HVQYNILLGE GPCEPGDCGG KLLCKHGVIG IVTAGGEGHV |
| dq473485-03 | WIEGSSYYPS RYQAGVMKGV GPAEPGDCGG ILRCIHGPIG LLTAGGGGYV |
| ef173422-03 | WIEGSSYYPS RYQAGVMKGV GPAEPGDCGG ILRCIHGPIG LLTAGGGGYV |
| dq473490-04 | WIEGNNYYPS RYQQGVMKGV GPAEPGDCGG ILRCVHGPIG LLTAGGSGFV |
| hrv-05 | WIEGNNYYPS RYQQGVMRGV GPAEPGDCGG ILRCVHGPIG LLTAGGSGYV |
| dq473486-06 | WIEGSSYYPS RYQTGVMKGV GPAEPGDCGG ILRCVHGPIG LLTAGGSGYV |
| l05355-14 | WIEGNPYYPS RFQAGVMKGV GPAEPGDCGG ILRCIHGPIG LLTAGGSGYV |
| k02121-14 | WIEGSPYYPS RFQAGVMKGV GPAELGDCGG ILRCIHGPIG LLTAEGSGYV |
| ef173420-17 | WIEGGNYYPS RFQSGVMKGV GPAEPGDCGG VLRCVHGPIG LLTAGGSGYV |
| hrv-26 | WIEGNNYYPS RYQQGVMRGV GPAEPGDCGG ILRCIHGPIG LLTAGGSGYV |
| hrv-27 | WIEGNNYYPS RYQQGVMKGV GPAEPGDCGG ILRCIHGPIG ILTAGGGGYV |
| ef173421-27 | WIEGNNYYPS RYQQGVMKGV GPAEPGDCGG ILRCIHGPIG ILTAGGGGYV |
| dq473487-35 | WIEGGPYYPS RYQVGVMKGV GPAEPGDCGG ILRCIHGPIG LLTAGGGGYV |
| hrv-35 | WIEGGPYYPS RYQVGVMKGV GPAEPGDCGG ILRCIHGPIG LLTAGGGGYV |
| ef173423-37 | WIEGSSYYPS RYQSGVMKGV GPAEPGDCGG ILRCVHGPIG LLTAGGSGYV |
| hrv-42 | WIEGNNYYPS RYQQGVMRGV GPAEPGDCGG ILRCIHGPIG LLTAGGSGYV |
| dq473488-48 | WIEGGNYYPS RYQAGVMKGV GPAEPGDCGG ILRCIHGPIG LLTAGGSGYV |
| hrv-52 | WIEGGSYYPS RFQSGVMKGV GPAEPGDCGG ILRCMHGPIG LLTAGGSGYV |
| hrv-52-f10 | WIEGGSYYPS RFQSGVMKGV GPAEPGDCGG ILRCVHGPIG LLTAGGSGYV |
| ef173424-52 | WIEGGSYYPS RFQSGVMKGV GPAEPGDCGG ILRCMHGPIG LLTAGGSGYV |
| hrv-69 | WIEGGNYYPS RFQSGVMKGI GPAEPGDCGG ILRCIHGPIG LLTAGGSGYV |
| dq473489-70 | WIEGGNYYPS RFQSGVMKGV GPAEPGDCGG ILRCVHGPIG LLTAGGSGYV |
| hrv-72 | WIEGGPYYPS RFQTGVMKGV GPAEPGDCGG ILRCVHGPIG LLTAGGSGYV |
| hrv-79 | WIEGGPYYPS RYQVGVMKGV GPAEPGDCGG ILRCVHGPIG LLTAGGSGYV |
| hrv-83 | WIEGGPYYPS RYQAGVMKGV GPAEPGDCGG ILRCVHGPIG LLTAGGGGYV |
| hrv-84 | WIEGNNYYPS RYQQGVMKGV GPAEPGDCGG ILRCVHGPIG LLTAGGGGFV |
| hrv-86 | WIEGGPYYPS RYQSGVMKGV GPAEPGDCGG ILRCIHGPIG LLTAGGSGFV |
| hrv-91 | WIEGGNYYPS RFQSGVMKGV GPAEPGDCGG ILRCVHGPIG LLTAGGSGYV |
| hrv-92 | WIEGGPYYPS RYQVGVMKGV GPAEPGDCGG ILRCVHGPIG LLTAGGSGYV |
| ef173425-93 | WIEGSNYYPS RYQQGVMKGV GPAEPGDCGG ILRCTHGPIG ILTAGGGGYV |
| hrv-97 | WIEGNSYYPS RYQQGVMKGV GPAEPGDCGG ILRCVHGPIG LLTAGGGGFV |
| hrv-99 | WIEGNNHYPS RYQQGVMKGM GPAEPGDCGG ILRCTHGPIG LLTAGGSGYV |
| | 1051 1100 |
| annotation | BBBBHHHH.. ......EQG. .......... .......... .......... |
| af499637-cva13 | AFADIRDLY. VEEEAMEQG. ITSYIESLGA AFGSGFTTQI SDKITELTSM |
| af546702-cva21 | AFADIRDLWV YEEEAMEQG. ITSYIESLGT AFGAGFTHTI SEKVTELTTM |
| v01149-pv1m | AFSDIRDLYA YEEEAMEQG. ITNYIESLGA AFGSGFTQQI SDKITELTNM |
| d00239-01 | AFTDLRQF.. ..QCAEEQG. ITDYIHMLGE AFGNGFVDSV KEQINAINPI |
| pico-01 | AFIDLRQF.. ..HCAEEQG. ITDYIHMLGE AFGNGFVDSV KEQINAINPI |
| hrv-01 | AFIDLRQF.. ..HCAEEQG. ITDYIHMLGE AFGNGFVDSV KEQINAINPI |
| x02316-02 | AFIDLRHF.. ..HCAEEQG. VTDYIHMLGE AFGNGFVDSV KEHIHAINPV |
| hrv-07 | AFIDLRKF.. ..QCAEEQG. ISDYVEHLGQ VFGVGFVDSI KQQVNFINPT |
| dq473503-07 | AFIDLRKF.. ..QCAEEQG. ISDYVEHLGQ VFGVGFVDSI KQQVNFINPT |
| hrv-08 | AFIDLRKYR. .ITEAEEQG. ITDYVKSLGD AFGVGFVEQI KEQVSNINPL |
| pico-09 | AFIDLRHF.. ..HCAEEQG. ITDYIHMLGE AFGNGFVDSV RDQVNAINPI |
| hrv-09 | AFIDLRHF.. ..HCAEEQG. ITDYIHMLGE AFGNGFVDSV RDQVNAINPI |
| hrv-09-f01 | AFIDLRHF.. ..HCAEEQG. ITDYIHMLGE AFGNGFVDSV REQVNAINPI |
| hrv-09-f02 | AFIDLRHF.. ..HCAEEQG. ITDYIHMLGE AFGNGFVDSV REQVNAINPI |
| dq473498-10 | AFIDLRQF.. ..HCADEQG. ITDYIHMLGE AFGTGFVDSV KEQINAINPI |
| hrv-10 | AFIDLRQF.. ..HCADEQG. ITDYIHMLGE AFGTGFVDSV KEQINAINPI |
| ef173414-11 | AFIDLRHF.. ..HCAEEQG. ITDYIHMLGE AFGSGFVDSV KDQINAINPI |
| ef173415-12 | AFIDLRDF.. ..HVADEQG. ISDYITNLGE AFGQGFMSQV KDQISELNPM |
| hrv-13 | AFIDLRHF.. ..LCAEEQG. VTDYIHMLGE AFGNGFVDSV KEHVNAINPI |
| hrv-13-f03 | AFIDLRHF.. ..LCAEEQG. VTDYIHMLGE AFGNGFVDSV KEHVNAINPI |
| dq473493-15 | AFIDLRHF.. ..HCAEEQG. VTDYIHMLGE AFGDGFVNSV KEQISAINPV |
| l24917-16 | AFIDLRHF.. ..HCAEEQG. ITDYIHMLGE AFGSGFVDSV KDQINSINPI |
| hrv-18 | AFIDLRHF.. ..QCAEEQG. ITDYIHMLGE AFGTGFVDSV KDQINAINPI |
| hrv-19 | AFIDLRHF.. ..HCAEEQG. ITDYIHMLGE AFGNGFVDSV RDQVNAINPI |
| hrv-20 | AFIDLRDF.. ..QVAEEQG. ISDYVHTLGE AFGAGFVDNI KEQIQAINPI |
| hrv-21 | AFIDLRHF.. ..HCAEEQG. ITDYIHMLGE AFGSGFVDSV KEQINAINPI |
| hrv-22 | AFIDLRHF.. ..HCAEEQG. ITDYIHMLGE AFGNGFVDSV REQVNAINPI |
| dq473497-23 | AFIDLRQF.. ..HCADEQG. ITDYIHMLGE AFGNGFVDSV KEHVHAINPI |
| hrv-24 | AFIDLRHF.. ..HCADEQG. ITDYIHMLGE AFGNGFVDSV KDQINAINPI |
| ef173416-24 | AFIDLRHF.. ..HCADEQG. ITDYIHMLGE AFGNGFVDSV KDQINAINPI |
| hrv-25 | AFIDLRQF.. ..HCADEQG. VTDYIHMLGE AFGAGFVDSV KEQINAINPI |
| dq473508-28 | AFIDLRDF.. ..LVAEEQG. VLDYVHTLGE AFGAGFVDNI KEQIQTINPI |
| hrv-29 | AFIDLRQF.. ..HCADEQG. ITDYIHMLGE AFGTGFVDSV KEQINAINPI |
| dq473512-30 | AFIDLRHF.. ..HCAEEQG. ITDYIHMLGE AFGNGFVDSV KEHVQAINPI |
| hrv-30 | AFIDLRHF.. ..HCAEEQG. ITDYIHMLGE AFGNGFVDSV KEHVQAINPI |
| hrv-31 | AFIDLRQF.. ..HCADEQG. ITDYIHMLGE AFGSGFVDSV KDQINAINPI |
| hrv-32 | AFIDLRHF.. ..HCAEEQG. ITDYVHMLGE AFGNGFVDSV RDQVNAINPI |
| hrv-33 | AFIDLRHF.. ..HCAEEQG. ITDYIHMLGE AFGSGFVDSV KDQINAINPI |
| hrv-34 | AFIDLRHF.. ..HCAEEQG. ITDYVHMLGE AFGLGFVDSV KDQINAINPI |
| dq473501-34 | AFIDLRHF.. ..HCAEEQG. ITDYVHMLGE AFGLGFVDSV KDQINAINPI |
| dq473505-36 | AFIDLRKF.. ..QCAEEQG. LSDYVEHLGQ VFGVGFVDSI KQQVNFINPT |
| hrv-38 | AFIDLRHF.. ..HCAEEQG. ITDYIHMLGE AFGNGFVDSV KEQVNAINPI |
| dq473495-38 | AFIDLRHF.. ..HCAEEQG. ITDYIHMLGE AFGNGFVDSV KEQVNAINPI |
| ay751783-39 | AFIDLRHF.. ..HCAEEQG. VTDYIHMLGE AFGNGFVDSV KEHVKAINPV |
| hrv-40 | AFIDLRHF.. ..HCAEEQG. ITDYIHMLGE AFGTGFVDSV KDQISAINPI |
| dq473491-41 | AFIDLRHF.. ..LCAEEQG. VTDYIHMLGE AFGNGFVDSV KEHVNAINPI |
| hrv-43 | AFIDLRHF.. ..HCAEEQG. ITDYIHLLGE AFGNGFVESV KEQVNAINPI |
| dq473499-44 | AFIDLRQF.. ..HCADEQG. ITDYIHMLGE AFGTGFVDSV KEQINAINPI |
| hrv-45 | AFTDLRKYR. .MVEAEEQG. ITDYVKSLGD AFGVGFVDQI KEQINNINPL |
| dq473506-46 | AFIDLRDF.. ..QIAEEQG. IPEYIHSLGE AFGSGFVDNI KDQIQSINPI |
| hrv-47 | AFIDLRQF.. ..HCADEQG. ITDYIHMLGE AFGSGFVDSV KDQINAINPI |
| dq473496-49 | AFIDLRHF.. ..HCAEEQG. ITDYIHMLGE AFGNGFVDSV KEHIHAINPV |
| hrv-49-f04 | AFIDLRHF.. ..HCAEEQG. VTDYIHMLGE AFGNGFVDSV KEHIHAINPV |
| hrv-50 | AFIDLRHF.. ..HCAEEQG. ITDYVHMLGE AFGLGFVDSV KDQINAINPI |
| hrv-51 | AFIDLRDF.. ..HVADTQG. ITDYVHNLGE AFGAGFIDNI REQIQVINPI |
| dq473507-53 | AFIDLRDF.. ..QVAEEQG. IPEYIHSLGE AFGSGFVDNI KDQIQTINPI |
| hrv-54 | AFIDLRHF.. ..HCAEEQG. ITDYIHMLGE AFGTGFVDSV KDQINAINPI |
| hrv-54-f05 | AFIDLRHF.. ..HCAEEQG. ITDYIHMLGE AFGTGFVDSV KDQINAINPI |
| dq473511-55 | AFIDLRHF.. ..HCAEEQG. ITDYIHMLGE AFGSGFVDSV KDQINAINPI |
| hrv-56 | AFIDLRHF.. ..HCAEEQG. ITDYIHMLGE AFGTGFVDSV KDQISAINPI |
| hrv-57 | AFIDLRHF.. ..HCAEEQG. ITDYIHMLGE AFGNGFVDSV KEQINAINPI |
| hrv-58 | AFIDLRKF.. ..QCAEEQG. LSDYVEHLGQ VFGVGFVDSI KEQVNFINPT |
| dq473500-59 | AFIDLRHF.. ..HCAEEQG. ITDYIHMLGE AFGTGFVDSV KEQISAINPV |
| hrv-60 | AFIDLRHF.. ..HCAEEQG. ITDYIHMLGE AFGNGFVDSV RDQVNAINPI |
| hrv-61 | AFIDLRHF.. ..LCAEEQG. VTDYIHMLGE AFGNGFVDSV KEHVNAINPV |
| hrv-62 | AFIDLRQF.. ..HCVDEQG. VTDYIHMLGE AFGAGFVDSV KEQINAINPI |
| hrv-63 | AFIDLRQF.. ..HCAEEQG. VTDYIHMLGE AFGTGFVDSV KEQINAINPI |
| hrv-64 | AFIDLRHF.. ..HCAEEQG. ITDYIHMLGE AFGNGFVDSV REQVNAINPI |
| ef173417-64 | AFIDLRHF.. ..HCAEEQG. ITDYIHMLGE AFGNGFVDSV REQVNAINPI |
| hrv-65 | AFIDLRDF.. ..HVADTQG. ITDYVHNLGE AFGAGFIDNI KEQIQVINPI |
| hrv-66 | AFIDLRHF.. ..HCAEEQG. ITDYIHTLGE AFGTGFVDSV KDQINAINPI |
| hrv-67 | AFIDLRHF.. ..HYAEEQG. ITDYIHMLGE AFGNGFVDSV KEQVNAINPI |
| hrv-68 | AFIDLRDF.. ..QVAEEQG. ISDYVHTLGE AFGAGFVDNI REQIQTINPI |
| hrv-71 | AFIDLRDF.. ..HVAETQG. ITDYIHNLGE AFGAGFIDNI KDQINVINPI |
| dq473492-73 | AFIDLRHF.. ..LCAEEQG. VTDYIHMLGE AFGNGFVDSV KEHVNAINPI |
| dq473494-74 | AFIDLRHF.. ..HCAEEQG. ITDYIHMLGE AFGNGFVDSV REQVNAINPI |
| dq473510-75 | AFIDLRQF.. ..HCAEEQG. ITDYIHMLGE AFGNGFVESV KEQVSAINPI |
| dq473502-76 | AFIDLRHF.. ..HCAEEQG. ITDYIHMLGE AFGSGFVDSV KDQINAINPI |
| hrv-76 | AFIDLRHF.. ..HCAEEQG. ITDYIHMLGE AFGSGFVDSV KDQINAINPI |
| hrv-77 | AFIDLRHF.. ..HCAEEQG. ITDYIHMLGE AFGTGFVDSV KDQINAINPI |
| hrv-78 | AFIDIRDF.. ..QCAEEQG. ISDYMQYLGE AFGTGFVDSV REHVQSINPI |
| ef173418-78 | AFIDIRDF.. ..QCAEEQG. ISDYMQYLGE AFGTGFVDSV REHVQSINPI |
| hrv-80 | AFIDLRDF.. ..HCAENQG. ISDYIHSLGE AFGAGFIDNI KDQIQTINPM |
| hrv-81 | AFIDLRHF.. ..HCAEEQG. ITDYIHMLGE AFGSGFVDSV KDQINSINPI |
| hrv-81-f06 | AFIDLRHF.. ..HCAEEQG. ITDYIHMLGE AFGSGFVDSV KEQINSINPI |
| hrv-81-f07 | AFIDLRHF.. ..HCAEEQG. ITDYIHMLGE AFGSGFVDSV KEQINSINPI |
| hrv-82 | AFIDLRHF.. ..HCAEEQG. ITDYIHMLGE AFGSGFVDSV KDQINAINPI |
| dq473509-82 | AFIDLRHF.. ..HCAEEQG. ITDYIHMLGE AFGSGFVDSV KDQINAINPI |
| hrv-85 | AFIDLRHF.. ..HCAEEQG. ITDYIHMLGE AFGTGFVDSV KEQISAINPI |
| pico-85 | AFIDLRHF.. ..HCAEEQG. ITDYIHMLGE AFGTGFVDSV KEQISAINPI |
| dq473504-88 | AFIDLRKF.. ..QCAEEQG. ISDYVEHLGQ VFGVGFVDSI KQQINFINPT |
| a10937-89 | AFIDLRKF.. ..QCAEEQG. LSDYVEHLGQ VFGVGFVDSI KQQVNFINPT |
| hrv-89 | AFIDLRKF.. ..QCAEEQG. LSDYVEHLGQ VFGVGFVDSI KQQVNFINPT |
| hrv-89-f08 | AFIDLRKF.. ..QCAEEQG. LSDYVEHLGQ VFGVGFVDSI KQQVNFINPT |
| hrv-89-f09 | AFIDLRKF.. ..QCAEEQG. LSDYVEHLGQ VFGVGFVDSI KQQVNFINPT |
| m16248-89 | AFIDLRKF.. ..QCAEEQG. LSDYVEHLGQ VFGVGFVDSI KQQVNFINPT |
| hrv-90 | AFIDLRHF.. ..HCAEEQG. ITDYIHMLGE AFGSGFVDSV KDQINAINPI |
| hrv-94 | AFIDLRHF.. ..HCAEEQG. ITDYIHMLGE AFGNGFVDSV REQVNAINPI |
| ef173419-94 | AFIDLRHF.. ..HCAEEQG. ITDYIHMLGE AFGNGFVDSV REQVNAINPI |
| hrv-95 | AFIDLRKYR. .ITEAEEQG. ITDYVKSLGD AFGVGFVEQI KEQVSNINPL |
| hrv-96 | AFIDLRHF.. ..LCAEEQG. VTDYIHMLGE AFGNGFVDSV KEHVNAINPV |
| hrv-98 | AFIDLRHF.. ..HCAEEQG. ITDYIHMLGE AFGTGFVDSV KDQINAINPI |
| hrv-100 | AFIDLRQF.. ..HCVEEQG. VTDYVHMLGE AFGMGFVDSV KDQINAINPI |
| ef186077-qpm | AFIDLRNY.. .SSLSEHQG. ITDYVTQLGN AFGDGFTSSI RETLVSACDV |
| ef077279-nat001 | AFIDLRNY.. .SSLSEHQG. ITDYVTQLGS AFGDGFTNSI RDTLVGACDV |
| ny1078 | AFIDLRNY.. .SSLSEHQG. VTDYITQLGS AFGDGFTSSI RQTLMGACDV |
| ef582385-c024 | AFTDLRPY.. .SCLDTHQGP IQDYVTQLGS AFGSGFTDTL KDQLTDVSAH |
| ef077280-nat045 | AFTDLRPY.. .NIKATSQGP VTNYLNQLGS AFGEGFTANI KDQLATVGNT |
| ef582387-c026 | AFIDLRNY.. .SSLSEHQG. ITDYVTQLGN AFGDGFTNSI RETLVSACDV |
| ef582386-c025 | AFTDLRPY.. .SSLAHTQGP ISDYITQLGN AFGTGFTQTL ETNLKETCSG |
| dq473485-03 | CFADIRQLD. ..FIADEQG. LGDYITSLGR AFGTGFTDQI SAKVCELQDV |
| ef173422-03 | CFADIRQLD. ..FIADEQG. LGDYITSLGR AFGTGFTDQI SAKVCELQDV |
| dq473490-04 | CFADIRMLE. .LFKAEYQG. ISDYIVNLGK AFGTGFTDHI SEKVAELQNI |
| hrv-05 | CFADIRALD. .LFKAEYQG. ISDYILNLGK AFGSGFTEHI SEKVTELQNI |
| dq473486-06 | CFADIRQLD. ..CIAEEQG. LNDYITNLGR AFGVGFTDQI SAKVTELQDV |
| l05355-14 | CFADIRQLE. ..CIAEEQG. LSDYITGLGR AFGVGFTDQI STKVTELQEV |
| k02121-14 | CFADIRQLE. ..CIAEEQG. LSDYITGLGR AFGVGFTDQI STKVTELQEV |
| ef173420-17 | CFADIRQLD. ..FIAETQG. ITDYITSLGK AFGVGFTDQI SAKITELQDS |
| hrv-26 | CFADIRTLD. .MFNAEYQG. ISDYILNLGK AFGTGFTEHI SEKVTELQNI |
| hrv-27 | CFADIRGLD. .MFQAEYQG. ISDYILNLGK AFGTGFTDHI SEKVSELQNI |
| ef173421-27 | CFADIRGLD. .MFQAEYQG. ISDYILNLGK AFGTGFTDHI SEKVSELQNI |
| dq473487-35 | CFADIRQLE. ..NVAEEQG. LGDYITSLGR AFGVGFTDQI SAKVVELQDV |
| hrv-35 | CFADIRQLE. ..NVAEEQG. LGDYITSLGR AFGVGFTDQI SAKVVELQDV |
| ef173423-37 | CFADIRQLD. ..CIAEEQG. LNDYITSLGR AFGVGFTDQI SAKVTELQDV |
| hrv-42 | CFADIRALD. .MFKAEYQG. ISDYISNLGK AFGTGFTEHI SEKVTELQNM |
| dq473488-48 | CFADIRQLD. ..FIAETQG. ITDYITSLGK AFGVGFTDQI SAKITELQDS |
| hrv-52 | CFADIRQLD. ..FIAETQG. ITDYITNLGK AFGVGFTDQI SAKVTELQDS |
| hrv-52-f10 | CFADIRQLD. ..FIAETQG. ITDYITNLGK AFGVGFTDQI SAKVTELQDS |
| ef173424-52 | CFADIRQLD. ..FIAETQG. ITDYITNLGK AFGVGFTDQI SAKVTELQDS |
| hrv-69 | CFADIRQLD. ..FIAETQG. ITDYITNLGK AFGVGFTDQI SAKVTELQDS |
| dq473489-70 | CFADIRQLD. ..FIAETQG. ITDYITSLGK AFGVGFTDQI SAKVTELQDS |
| hrv-72 | CFADIRQLE. ..CIAEEQG. ISDYITSLGR AFGVGFTDQI SAKVTELQDV |
| hrv-79 | CFADIRQLE. ..QIAEEQG. LGDYITSLGR AFGVGFTDQI SAKVTELQDV |
| hrv-83 | CFADIRQLE. ..CIAEEQG. LGDYITSLGR AFGVGFTDQI SAKVTELQDV |
| hrv-84 | CFADIRMLD. .MLHAHYQG. IPDYITNLGR AFGTGFTDHI NDKIAELQST |
| hrv-86 | CFADIRELE. ..CIAEEQG. ISDYISNLGK AFGAGFTDQI TSKIGELQDT |
| hrv-91 | CFADIRQLD. ..LIAETQG. ITDYITSLGK AFGVGFTDQI SAKVSELQDS |
| hrv-92 | CFADIRQLD. ..CIAEEQG. LGDYITSLGR AFGVGFTDQI SAKVTELQDV |
| ef173425-93 | CFADIRGLD. .MFHAEYQG. ISDYILNLGK AFGTGFTDHI SEKVSELQNI |
| hrv-97 | CFADIRGLD. .MFRAEYQG. ISDYILNLGK AFGTGFTDHI SEKVSELQNI |
| hrv-99 | CFADIRALD. .MFEAEYQG. ISDYILNLGK AFGTGFTEHI SEKVTELQNI |
| | 1101 1150 |
| annotation | .......... .......... .......... .......... .......... |
| af499637-cva13 | VTSVLTEKLL KNLIKLISSL VIITRNYEDT TTVLATLALL GCDVSPWAWL |
| af546702-cva21 | VTSTITEKLL KNLVKIVSAL VIVVRNYEDT TTILATLALL GCDISPWQWL |
| v01149-pv1m | VTSTITEKLL KNLIKIISSL VIITRNYEDT TTVLATLALL GCDASPWQWL |
| d00239-01 | NN..ISKKVI KWLLRIISAM VIIIRNSSDP QTIIATLTLI GCNGSPWRFL |
| pico-01 | NN..ISKKVI KWLLRIISAM VIIIRNSSDP QTIIATLTLI GCNGSPWRFL |
| hrv-01 | NN..ISKKVI KWLLRIISAM VIIIRNSSDP QTIIATLTLI GCNGSPWRFL |
| x02316-02 | GN..ISKKII KWMLRIISAM VIIIRNSSDP QTILATLTLI GCSGSPWRFL |
| hrv-07 | SK..IGSKVI KWLLRIVSAM IIMVRNSSDP QTIVATLTLL GCSGSPWRFL |
| dq473503-07 | SK..IGSKVI KWLLRIVSAM IIMVRNSSDP QTIVATLTLL GCSGSPWRFL |
| hrv-08 | NN..ISAKVI KWLIRVISAL VIAVRSQGDL ATLSATLVLL GCSDSPWRFL |
| pico-09 | NS..ISKKIV KWMLRIISAM VIIIRNSSDP QTIIATLTLI GCSGSPWRFL |
| hrv-09 | NN..ISKKIV KWMLRIISAM VIIIRNSSDP QTIIATLTLI GCSGSPWRFL |
| hrv-09-f01 | NN..ISKKII KWMLRIISAM VIIIRNSSDP QTIIATLTLI GCSGSPWRFL |
| hrv-09-f02 | NN..ISKKII KWMLRIISAM VIIIRNSSDP QTIIATLTLI GCSGSPWRFL |
| dq473498-10 | NN..ISKKII KWLLRIISAM VIIIRNSSDP QTIIATLTLI GCSGSPWRFL |
| hrv-10 | NN..ISKKII KWLLRIISAM VIIIRNSSDP QTIIATLTLI GCSGSPWRFL |
| ef173414-11 | NN..ISRKIV KWILRIISAM VVIIRNSSDP QTIIATLTLL GCSGSPWRFL |
| ef173415-12 | NK..ITGKVL KWTIRIISAL TIVVRNSSDP LTIVATLAML GCNGSPWRFL |
| hrv-13 | NN..ISKKVI KWLLRIVSAM VIIIRNSSDP HTVVATLTLI GCSGSPWRFL |
| hrv-13-f03 | NN..ISKKVI KWLLRIVSAM VIIIRNSSDP HTVVATLTLI GCSGSPWRFL |
| dq473493-15 | NN..ISKKIV KWMLRIISAM VIIIRNSSDP QTVIATLTLI GCSGSPWRFL |
| l24917-16 | NN..ISSKMV KWMLRIISAM VIIIRNSSDP QTIIATLTLI GCNGSPWRFL |
| hrv-18 | NN..ISKKII KWLLRIISAM VIIIRNSSDP QTIIATLTLI GCSGSPWRFL |
| hrv-19 | NN..ISKKLI KWMLRIISAM VIIIRNSSDP QTIVATLTLV GCSGSPWRFL |
| hrv-20 | NK..ITSKII KWTIRIVSAI TIIIRNNADP HTIIATLALL GCSGSPWRFI |
| hrv-21 | NN..ISKKII KWLLRIISAM VIIIRNSSDP QTIIATLTLI GCSGSPWRFL |
| hrv-22 | NN..ISKKLV KWLLRIVSAM VIIIRNSSDP QTVIATLTLI GCSGSPWRFL |
| dq473497-23 | GN..ISKKIV KWMLRIISAM VIIIRNSSDP QTILATLTLI GCSGSPWRFL |
| hrv-24 | NS..ISKKII KWLLRIISAM VIIIRNSSDP QTIVATLTLI GCSGSPWRFL |
| ef173416-24 | NS..ISKKII KWLLRIISAM VIIIRNSSDP QTIVATLTLI GCSGSPWRFL |
| hrv-25 | NN..ISKKII KWLLRIISAM VIIIRNSSDP QTIIATLTLI GCSGSPWRFL |
| dq473508-28 | NK..ITSKII KWTIRIISAI TIIIRNNADP HTIIATLALL GCSGSPWRFI |
| hrv-29 | NN..ISKKII KWLLRVISAM VIIIRNSSDP QTIIATLTLI GCSGSPWRFL |
| dq473512-30 | GN..ISKKII KWMLRVISAM VIIIKNSSDP QTILATLTLI GCSGSPWRFL |
| hrv-30 | GN..ISKKII KWMLRVISAM VIIIRNSSDP QTILATLTLI GCSGSPWRFL |
| hrv-31 | NN..ISKKII KWLLRIISAM VIIIRNSSDP QTIVATLTLI GCSGSPWRFL |
| hrv-32 | NS..ISKKII KWMLRIISAM VIIIRNSSDP QTIIATLTLI GCSGSPWRFL |
| hrv-33 | NN..ISRKIV KWILRIISAM VVIIRNSSDP QTIIATLTLI GCSGSPWRFL |
| hrv-34 | NG..ISKKII KWLLRIISAM VIIIRNSSDP QTIIATLTLI GCSGSPWRFL |
| dq473501-34 | NG..ISKKII KWLLRIISAM VIIIRNSSDP QTIIATLTLI GCSGSPWRFL |
| dq473505-36 | SK..IGSKVI KWLLRIVSAM IIMVRNSSDP QTIIATLTLL GCSGSPWRFL |
| hrv-38 | NN..VSKKII KWMLRIISAM VIIIRNSSDP QTIIATLTLI GCSGSPWRFL |
| dq473495-38 | NN..VSKKII KWMLRIISAM VIIIRNSSDP QTIIATLTLI GCSGSPWRFL |
| ay751783-39 | GN..ISKKII KWMLRIISAM VIIIRNSSDP QTILATLTLI GCSGSPWRFL |
| hrv-40 | NN..ISKKVI KWLLRIISAM VIIIRNSSDP QTIIATLTLI GCSGSPWRFL |
| dq473491-41 | NN..ISKKVI KWLLRIVSAM VIIIRNSSDP HTVVATLTLI GCSGSPWRFL |
| hrv-43 | NN..ISKKMV KWMLRIISAM VIIIRNSSDP QTIIATLTLI GCSGSPWRFL |
| dq473499-44 | NN..ISKKII KWLLRIISAM VIIIRNSSDP QTIIATLTLI GCSGSPWRFL |
| hrv-45 | NK..ISAKVI KWLIRVISAL VIAVRSQGDP ATLSATLLLL GCSNSPWRFL |
| dq473506-46 | NK..ITSKII KWVIRIISAV TIIIRNSADP HTIVATLALL GCSGSPWRFI |
| hrv-47 | NN..ISKKII KWLLRIISAM VIIIRNSSDP QTIIATLTLI GCSGSPWRFL |
| dq473496-49 | GN..VSKKII KWMLRIISAM VIIIRNSSDP QTILATLTLI GCSGSPWRFL |
| hrv-49-f04 | GN..ISKKII KWMLRIISAM VIIIRNSSDP QTILATLTLI GCSGSPWRFL |
| hrv-50 | NS..ISKKII KWLLRIISAM VIIIRNSSDP QTIIATLTLI GCSGSPWRFL |
| hrv-51 | NK..ITGKIV KWIIRIISAI TIIIRNSADP HTVVATLALI GCSGSPWRFI |
| dq473507-53 | NK..ISSKIV KWVIRIISAI TIIIRNNADP HTIVATLALL GCSGSPWRFI |
| hrv-54 | NS..ISKKVI KWLLRIISAM VIIIRNSSDP QTIIATLTLI GCSGSPWRFL |
| hrv-54-f05 | NN..ISKKII KWLLRIISAM VIIIRNSSDP QTIIATLTLI GCSGSPWRFL |
| dq473511-55 | NN..ISKKII KWLLRIISAM VIIIRNSSDP QTIIATLTLI GCSGSPWRFL |
| hrv-56 | NN..ISKKII KWLLRIISAM VIIIRNSSDP QTIIATLTLI GCSGSPWRFL |
| hrv-57 | NS..ISKKII KWLLRIISAM VIIIRNSSDP QTIIATLTLI GCSGSPWRFL |
| hrv-58 | SK..IGSKVV KWLLRIVSAM IIMVRNSSDP QTIIATLTLL GCSGSPWKFL |
| dq473500-59 | NN..ISKKII KWMLRIISAM VIIIRNSSDP QTIIATLTLI GCSGSPWRFL |
| hrv-60 | NN..ISKKLI KWMLRIISAM VIIIRNSSDP QTIVATLTLI GCSGSPWRFL |
| hrv-61 | NN..ISKKVI KWLLRIVSAM VIIIRNSSDP QTVVATLTLI GCSGSPWRFL |
| hrv-62 | NS..ISKKII KWLLRIISAM VIIIRNSSDP QTIIATLTLI GCSGSPWRFL |
| hrv-63 | NN..ISKKII KWMLRIISAM VIIIRNSSDP QTIIATLTLI GCSGSPWRFL |
| hrv-64 | NN..ISKKLI KWLLRIVSAM VIIIRNSSDP QTVIATLTLI GCSGSPWRFL |
| ef173417-64 | NN..ISKKLI KWLLRIVSAM VIIIRNSSDP QTVIATLTLI GCSGSPWRFL |
| hrv-65 | NK..VTSKIV KWIIRIISAI TIIVRNNADP HTIIATLALL GCSGSPWRFI |
| hrv-66 | NN..ISKKII KWLLRIISAM VIIIRNSSDP QTIIATLTLI GCSGSPWRFL |
| hrv-67 | NN..ISKKII KWMLRIISAM VIIIRNSSDP QTIIATLTLI GCSGSPWRFL |
| hrv-68 | NK..ITSKIV KWTIRIVSAI TIIIRNNADP HTIVATLALL GCSGSPWRFI |
| hrv-71 | NK..ITSKIV KWIIRIISAM TIIIRNSADP HTIIATLALI GCSGSPWRFI |
| dq473492-73 | NS..ISKKVI KWLLRIVSAM VIIIRNSSDP HTVIATLTLI GCSGSPWRFL |
| dq473494-74 | NN..ISKKIV KWMLRIISAM VIIIRNCSDP QTIIATLTLI GCSGSPWRFL |
| dq473510-75 | NN..ISKKIV KWILRIISAM VIIIRNNSDP QTIIATLTLI GCSGSPWRFL |
| dq473502-76 | NN..ISRKIV KWILRIISAM VVIIRNSSDP QTIIATLTLI GCSGSPWRFL |
| hrv-76 | NN..ISRKIV KWILRIISAM VVIIRNSSDP QTIIATLTLI GCSGSPWRFL |
| hrv-77 | NN..ISKKII KWLLRIISAM VIIIRNSSDP QTIIATLTLI GCSGSPWRFL |
| hrv-78 | NK..INSKMV KWLLRIISAM VIIIRNNADP QTIIATLTLL GCSGSPWRFL |
| ef173418-78 | NK..INSKMV KWLLRIISAM VIIIRNNADP QTIIATLTLL GCSGSPWRFL |
| hrv-80 | NK..ITSKIV KWLIRIISAV TIIIRNNADP HTIIATLALI GCSGSPWRFI |
| hrv-81 | NS..ISSKMV KWMLRIISAM VIIIRNSSDP QTIIATLTLI GCNGSPWRFL |
| hrv-81-f06 | NS..ISSKMV KWMLRIISAM VIIIRNSSDP QTIIATLTLI GCNGSPWRFL |
| hrv-81-f07 | NS..ISSKMV KWMLRIISAM VIIIRNSSDP QTIIATLTLI GCNGSPWRFL |
| hrv-82 | NN..ISKKLV KWILRIISAM VIIIRNSSDP QTVIATLTLI GCSGSPWRFL |
| dq473509-82 | NN..ISKKLV KWILRIISAM VIIIRNSSDP QTVIATLTLI GCSGSPWRFL |
| hrv-85 | NN..ISKKII KWLLRIISAM VIIIRNSSDP QTIIATLTLI GCSGSPWRFL |
| pico-85 | NN..ISKKII KWLLRIISAM VIIIRNSSDP QTIIATLTLI GCSGSPWRFL |
| dq473504-88 | SK..IGSKVI KWLLRIVSAM IIMIRNSSDP QTIVATLTLL GCSGSPWRFL |
| a10937-89 | SK..IGSKVI KWLLRIVSAM IIMVRNSSDP QTVIATLTLL GCSGSPWRFL |
| hrv-89 | SK..IGSKVI KWLLRIVSAM IIMVRNSSDP QTVIATLTLL GCSGSPWRFL |
| hrv-89-f08 | SK..IGSKVI KWLLRIVSAM IIMVRNSSDP QTVIATLTLL GCSGSPWRFL |
| hrv-89-f09 | SK..IGSKVI KWLLRIVSAM IIMVRNSSDP QTVIATLTLL GCSGSPWRFL |
| m16248-89 | SK..IGSKVI KWLLRIVSAM IIMVRNSSDP QTVIATLTLL GCSGSPWRFL |
| hrv-90 | NN..ISKKII KWLLRIISAM VIIIRNSSDP QTIIATLTLI GCSGSPWRFL |
| hrv-94 | NN..ISKKLV KWLLRIVSAM VIIIRNSSDP QTVIATLTLI GCSGSPWRFL |
| ef173419-94 | NN..ISKKLV KWLLRIVSAM VIIIRNSSDP QTVIATLTLI GCSGSPWRFL |
| hrv-95 | NK..ISAKVI KWLIRVISAL VIAVRSQGDL ATLSATLVLL GCSDSPWRFL |
| hrv-96 | NS..ISKKVI KWLLRIVSAM VIIIRNSSDP QTVVATLTLI GCSGSPWRFL |
| hrv-98 | NN..ISKKII KWLLRIISAM VIIIRNSSDP QTIIATLTLI GCSGSPWRFL |
| hrv-100 | NG..ISKKII KWLLRIISAM VIIIRNSSDP QTIIATLTLI GCSGSPWRFL |
| ef186077-qpm | S.DKLTGKII KWTIRLISAL SIIVRNSTDT PTVIATLALL GCSGSPWRYL |
| ef077279-nat001 | S.DKLTGKII KWTIRLISAL SIIVRNSSDT PTVIATLALL GCSGSPWRYL |
| ny1078 | S.DKLTSKII KWTIRLISAL SIIVRNSTDT PTIIATLALL GCSGSPWRYL |
| ef582385-c024 | IKDKITSKVV KWLVRIISAL TIMIRNSTDT PTVLATLALL GCHKSPWSFL |
| ef077280-nat045 | IMDGLTNKVV KWLIRIISAM TIMIRNSSDT PTVLATLALL GCHESPWAFL |
| ef582387-c026 | S.DKLTGKII KWTIRLISAL SIIVRNSTDT PTIIATLALL GCSGSPWRYL |
| ef582386-c025 | MFDAITSKTV KWIVRIISAL TIVIRNSSDT PTVLATMALL GCTGSPWQYL |
| dq473485-03 | AKDFLTTKVL SKVVKMISAL VIICRNHDDL VTVTATLALL GCDGSPWRFL |
| ef173422-03 | AKDFLTTKVL SKVVKMISAL VIICRNHDDL VTVTATLALL GCDGSPWRFL |
| dq473490-04 | SMGYLNTKVL SKIIKIVSAL VIICRNHDDL ITVTATLALL GCDGSPWRYL |
| hrv-05 | SMGYLNTKVL SKIVKIVSAL VIICRNHDDL ITVTATLALL GCDGSPWRYL |
| dq473486-06 | AKDFLTTKVL SKVVKMISAL VIICRNHDDL VTVTATLALL GCDGSPWRFL |
| l05355-14 | AKDFLTTKVL SKVVKMVSAL VIICRNHDDL VTVTATLALL GCDGSPWRFL |
| k02121-14 | AKDFLTTKVL SKVVKMVSAL VIICRNHDDL VTVTATLALL GCDGSPWRFL |
| ef173420-17 | AKDYINVKIL SKVVKMISAL VIICRNHEDL VTVTATLALL GCDGSPWRFL |
| hrv-26 | SMGYLNTKVL SKIVKIISAL VIICRNHDDL ITVTATLALL GCDGSPWRYL |
| hrv-27 | TMGCLNTKVL SKVIKIVSAL VIICRNHEDL VTVTATLALL GCDGSPWRFL |
| ef173421-27 | TMGCLNTKVL SKVIKIVSAL VIICRNHEDL VTVTATLALL GCDGSPWRFL |
| dq473487-35 | AKDFLTTKVL SKVVKMISAL VIICRNHDDL VTVTATLALL GCDGSPWRFL |
| hrv-35 | AKDFLTTKVL SKVVKMISAL VIICRNHDDL VTVTATLALL GCDGSPWRFL |
| ef173423-37 | AKDFLTTKVL SKVVKMISAL VIICRNHDDL VTVTATLALL GCDGSPWRFL |
| hrv-42 | SLGYLNTKVL SKIVKIVSAL VIICRNHDDL ITVTATLALL GCDGSPWRYL |
| dq473488-48 | AKDYINVKIL SKVVKMVSAL VIICRNHEDL ITVTATLALL GCDGSPWRFL |
| hrv-52 | VKDYINVKIL SKVVKMVSAL VIICRNHDDL VTVTATLALL GCDGSPWRFL |
| hrv-52-f10 | VKDYINVKIL SKVVKMVSAL VIICRNHDDL VTVTATLALL GCDGSPWRFL |
| ef173424-52 | VKDYINVKIL SKVVKMVSAL VIICRNHDDL VTVTATLALL GCDGSPWRFL |
| hrv-69 | VKDYINVKIL SKVVKMISAL VIICRNHDDL ITVTATLALL GCDGSPWRFL |
| dq473489-70 | VKDYINVKIL SKVVKMISAL VIICRNHEDL VTVTATLALL GCDGSPWRFL |
| hrv-72 | AKDFLTTKVL SKVVKMVSAL VIICRNYEDL VTVTATLALL GCDGSPWRFL |
| hrv-79 | AKDFLTTKVL SKVVKMISAL VIICRNHDDL VTVTATLALL GCDGSPWRFL |
| hrv-83 | AKDFLTTKVL SKVVKMISAL VIICRNHDDL VTVTATLALL GCDGSPWRFL |
| hrv-84 | TTNYLNTKVL SKVVKIVSAL VIICRNHDDL VTVTATLALL GCDGSPWRFL |
| hrv-86 | VKDYLTTKML SKVVKMVSAL VIICRNHEDL VTVTATLALL GCDGSPWRFL |
| hrv-91 | VKDYINVKIL SKVVKMISAL VIICRNHEDL VTVTATLALL GCDGSPWRFL |
| hrv-92 | AKDFLTTKVL SKVVKMISAL VIICRNHDDL VTVTATLALL GCDGSPWRFL |
| ef173425-93 | TMGYLNTKVL SKVIKIVSAL VIICRNHDDL VTVTATLALL GCDGSPWRFL |
| hrv-97 | TMGYLNTKVL SKVIKIVSAL VIICRNHEDL VTVTATLALL GCDGSPWRFL |
| hrv-99 | SMGYLNTKVL SKIVKIVSAL VIICRNHDDL ITVTATLALL GCDGSPWRYL |
| | 1151 1200 |
| annotation | .......... ......ES.. .......... .......... .......... |
| af499637-cva13 | KKKVCDILEI .PFVLKQG.D SWMKKFTEAC NAAKGLEWIS NKISKFIDWI |
| af546702-cva21 | KKKACDLLEI .PYVMRQG.D GWMKKFTEAC NAAKGLEWIS NKISKFVDWL |
| v01149-pv1m | RKKACDVLEI .PYVIKQG.D SWLKKFTEAC NAAKGLEWVS NKISKFIDWL |
| d00239-01 | KEKFCKWTQL .TYIHKES.D SWLKKFTEMC NAARGLEWIG NKISKFIDWM |
| pico-01 | KEKFCKWTQL .TYIHKES.D SWLKKFTEMC NAARGLEWIG NKISKFIDWM |
| hrv-01 | KEKFCKWTQL .TYIHKES.D SWLKKFTEMC NAARGLEWIG NKISKFIDWM |
| x02316-02 | KEKFCKWTQL .NYIHKES.D SWLKKFTEAC NAARGLEWIG NKISKFIEWM |
| hrv-07 | KEKFCSWLQL .NYIHKES.D SWLKKFTEAC NAARGLEWIG QKISKFIDWI |
| dq473503-07 | KEKFCSWLQL .NYIHKES.D SWLKKFTEAC NAARGLEWIG QKISKFIDWI |
| hrv-08 | KQKVCQWLDL .RYVHKES.D GWIKKFTEMC NAARGLEWIG CKISKFIDWL |
| pico-09 | KEKFCKWTQL .NYVHKES.D SWLKKFTEMC NAARGLEWIG NKISKFIDWM |
| hrv-09 | KEKFCKWTQL .NYVHKES.D SWLKKFTEMC NAARGLEWIG NKISKFIDWM |
| hrv-09-f01 | KEKFCKWTQL .NYVHKES.D SWLKKFTEMC NAARGLEWIG NKISKFIDWM |
| hrv-09-f02 | KEKFCKWTQL .NYVHKES.D SWLKKFTEMC NAARGLEWIG NKISKFIDWM |
| dq473498-10 | KEKFCKWTQL .TYIHKES.D SWLKKFTEMC NAARGLEWIG NKISKFIEWM |
| hrv-10 | KEKFCKWTQL .TYIHKES.D SWLKKFTEMC NAARGLEWIG NKISKFIEWM |
| ef173414-11 | KEKFCKWTQL .NYVHKES.D TWLKKFTEMC NAARGLEWIG NKISKFIEWM |
| ef173415-12 | KEKICKWFQL .NYVHKES.D GWLKKFTEMC NAARGLEWIG NKISKFIDWL |
| hrv-13 | KEKFCKWTQL .NYIHKES.D SWLKKFTEMC NAARGLEWIG NKISKFIEWM |
| hrv-13-f03 | KEKFCKWTQL .NYIHKES.D SWLKKFTEMC NAARGLEWIG NKISKFIEWM |
| dq473493-15 | KEKFCKWTQL .NYVHKES.D SWLKKFTEMC NAARGLEWIG NKISKFIEWM |
| l24917-16 | KEKFCKWTQL .TYIHKES.D SWLKKFTEMC NAARGLEWIG NKISKFIDWM |
| hrv-18 | KEKFCKWTQL .NYIHKES.D SWLKKFTEMC NAARGLEWIG NKISKFIEWM |
| hrv-19 | KEKFCKWTQL .NYIHKES.D SWLKKFTEMC NAARGLEWIG NKISKFIDWM |
| hrv-20 | KEKVCNWLQL .VYVHKES.D GWIKKFTEMC NAARGLEWLG NKISKFIDWL |
| hrv-21 | KEKFCKWTQL .NYIHKES.D SWLKKFTEMC NAARGLEWIG NKISKFIDWM |
| hrv-22 | KEKFCKWTQL .NYVHKES.D SWLKKFTEMC NAARGLEWIG NKISKFIDWM |
| dq473497-23 | KEKFCKWTQL .SYIHKES.D SWLKKFTEAC NAARGLEWIG NKISKFIEWM |
| hrv-24 | KEKFCKWTQL .NYIHKES.D SWLKKFTEMC NAARGLEWIG NKISKFIEWM |
| ef173416-24 | KEKFCKWTQL .NYIHKES.D SWLKKFTEMC NAARGLEWIG NKISKFIEWM |
| hrv-25 | KEKFCKWTQL .NYIHKES.D SWLKKFTEMC NAARGLEWIG NKISKFIEWM |
| dq473508-28 | KEKACNWLQL .VYIHKES.D GWIKKFTEMC NAARGLEWLG NKISKFIDWL |
| hrv-29 | KEKFCKWTQL .NYIHKES.D SWLKKFTEMC NAARGLEWIG NKISKFIEWM |
| dq473512-30 | REKFCKGTQL .SYIHKES.D SWLKKFTEAC NAARGLEWIG NKISKFIEWM |
| hrv-30 | KEKFCKWTQL .SYIHKES.D SWLKKFTEAC NAARGLEWIG NKISKFIEWM |
| hrv-31 | KERFCKWTQL .NYIHKES.D SWLKKFTEMC NAARGLEWIG NKISKFIEWM |
| hrv-32 | KEKFCKWTQL .NYVHKES.D SWLKKFTEMC NAARGLEWIG NKISKFIDWM |
| hrv-33 | KEKFCKWTQL .NYIHKES.D SWLKKFTEMC NAARGLEWIG NKISKFIEWM |
| hrv-34 | KEKFCKWTQL .NYIHKES.D SWLKKFTEMC NAARGLEWIG NKISKFIDWM |
| dq473501-34 | KEKFCKWTQL .NYIHKES.D SWLKKFTEMC NAARGLEWIG NKISKFIDWM |
| dq473505-36 | KEKLCAWLQL .SYVHKQS.D SWLKKFTEAC NAARGLEWIG QKISKFIDWI |
| hrv-38 | KEKFCKWTQL .NYIHKES.D SWLKKFTEMC NAARGLEWIG NKISKFIDWM |
| dq473495-38 | KEKFCKWTQL .NYIHKES.D SWLKKFTEMC NAARGLEWIG NKISKFIDWM |
| ay751783-39 | KEKFCKWTQL .TYIHKES.D SWLKKFTEMC NAARGLEWIG NKISKFIEWM |
| hrv-40 | KEKFCKWTEL .TYIHKES.D SWLKKFTEMC NAARGLEWIG NKISKFIDWM |
| dq473491-41 | KEKFCKWTQL .NYIHKES.D SWLKKFTEMC NAARGLEWIG NKISKFIEWM |
| hrv-43 | KEKFCKWTQL .NYIHKES.D SWLKKFTEMC NAARGLEWIG NKISKFIEWM |
| dq473499-44 | KEKFCKWTQL .NYIHKES.D SWLKKFTEMC NAARGLEWIG NKISKFIEWM |
| hrv-45 | KQKVCTWLGL .RYIHKES.D GWIKKFTEMC NAARGLEWIG CKISKFIDWL |
| dq473506-46 | KEKVCGWLQL .TYIHKES.D GWIKKFTEMC NAARGLEWLG NKISKFIDWL |
| hrv-47 | KEKFCKWTQL .NYIHKES.D SWLKKFTEMC NAARGLEWIG NKISKFIEWM |
| dq473496-49 | KEKFCKWTQL .NYIHKES.D SWLKKFTEAC NAARGLEWIG NKISKFIEWM |
| hrv-49-f04 | KEKFCKWTQL .NYIHKES.D SWLKKFTEAC NAARGLEWIG NKISKFIEWM |
| hrv-50 | KEKFCKWTQL .NYIHKES.D SWLKKFTEMC NAARGLEWIG NKISKFIEWM |
| hrv-51 | KEKVCNWLQL .SYVHKES.D GWIKKFTEMC NAARGLEWLG NKISKFIDWV |
| dq473507-53 | KEKVCGWLQL .NYIHKES.D GWIKKFTEMC NAARGLEWLG NKISKFIDWL |
| hrv-54 | KEKFCKWTQL .NYIHKES.D SWLKKFTEMC NAARGLEWIG NKISKFIDWM |
| hrv-54-f05 | KEKFCKWTQL .NYIHKES.D SWLKKFTEMC NAARGLEWIG NKISKFIDWM |
| dq473511-55 | KEKFCKWTQL .NYVHKES.D SWLKKFTEMC NAARGLEWIG NKISKFIDWM |
| hrv-56 | KEKFCKWTQL .NYVHKES.D SWLKKFTEMC NAARGLEWIG NKISKFIEWM |
| hrv-57 | KEKFCKWTQL .NYIHKES.D SWLKKFTEMC NAAKGLEWIG NKISKFIDWM |
| hrv-58 | KEKFCAWLQL .NYVHKQS.D SWLKKFTEAC NAARGLEWIG QKISKFIDWI |
| dq473500-59 | KEKFCKWTQL .NYIHKES.D TWLKKFTEMC NAARGLEWIG NKISKFIEWM |
| hrv-60 | KEKFCKWTQL .TYIHKES.D SWLKKFTEMC NAARGLEWIG NKISKFIDWM |
| hrv-61 | KEKFCKWTQL .TYIHKES.D SWLKKFTEMC NAARGLEWIG NKISKFIEWM |
| hrv-62 | KEKFCKWTQL .NYIHKES.D SWLKKFTEMC NAARGLEWIG NKISKFIEWM |
| hrv-63 | KEKFCKWTQL .NYIHKES.D TWLKKFTEMC NAARGLEWIG NKISKFIEWM |
| hrv-64 | KEKFCKWTQL .NYVHKES.D SWLKKFTEMC NAARGLEWIG NKISKFIDWM |
| ef173417-64 | KEKFCKWTQL .NYVHKES.D SWLKKFTEMC NAARGLEWIG NKISKFIDWM |
| hrv-65 | KEKVCNWLHL .NYIHKES.D GWIKKFTEMC NAARGLEWLG NKISKFIDWF |
| hrv-66 | KEKFCKWTQL .NYIHKES.D SWLKKFTEMC NAARGLEWIG NKISKFIDWM |
| hrv-67 | KEKFCKWTQL .NYVHKES.D SWLKKFTEMC NAARGLEWIG NKISKFIDWM |
| hrv-68 | KEKICNWLQL .VYIHKES.D GWIKKFTEMC NAARGLEWLG NKISKFIDWL |
| hrv-71 | KDKVCKWLQL .TYVHKES.D GWIKKFTEMC NAARGLEWLG NKISKFIDWF |
| dq473492-73 | KEKFCKWTQL .NYIHKES.D SWLKKFTEMC NAARGLEWIG NKISKFIEWM |
| dq473494-74 | KEKFCKWTQL .SYIHKES.D SWLKKFTEMC NAARGLEWIG NKISKFIDWM |
| dq473510-75 | KEKFCKWTQL .SYIHKES.D SWLKKFTEMC NAARGLEWIG NKISKFIEWM |
| dq473502-76 | KEKFCKWTQL .NYIHKES.D SWLKKFTEMC NAARGLEWIG NKISKFIEWM |
| hrv-76 | KEKFCKWTQL .NYIHKES.D SWLKKFTEMC NAARGLEWIG NKISKFIEWM |
| hrv-77 | KEKFCKWTQL .NYVHKES.D SWLKKFTEMC NAARGLEWIG NKISKFIEWM |
| hrv-78 | KNKFCNWFGL .TYVHKES.D SWLKKFTEMC NAARGLEWIG NKISKFIDWI |
| ef173418-78 | KNKFCNWFGL .TYVHKES.D SWLKKFTEMC NAARGLEWIG NKISKFIDWI |
| hrv-80 | KEKVCNWLQL .NYVHKES.D NWVKKFTEMC NAARGLEWLG NKISKFIDWV |
| hrv-81 | KEKFCKWTQL .TYIHKES.D SWLKKFTEMC NAARGLEWIG NKISKFIDWM |
| hrv-81-f06 | KEKFCKWTQL .TYVHKES.D SWLKKFTEMC NAARGLEWIG NKISKFIDWM |
| hrv-81-f07 | KEKFCKWTQL .TYVHKES.D SWLKKFTEMC NAARGLEWIG NKISKFIDWM |
| hrv-82 | KEKFCKWTQL .HYVHKES.D SWLKKFTEMC NAARGLEWIG NKISKFIEWM |
| dq473509-82 | KEKFCKWTQL .HYVHKES.D SWLKKFTEMC NAARGLEWIG NKISKFIEWM |
| hrv-85 | KEKFCKWTQL .TYIHKES.D SWLKKFTEMC NAARGLEWIG NKISKFIEWM |
| pico-85 | KEKFCKWTQL .TYIHKES.D SWLKKFTEMC NAARGLEWIG NKISKFIEWM |
| dq473504-88 | KEKFCSWLQL .NYVHKES.D SWLKKFTEAC NAARGLEWIG QKISKFIDWI |
| a10937-89 | KEKLCAWLQL .SYVHKQS.D SWLKKFTEAC NAARGLEWIG QKISKFIDWI |
| hrv-89 | KEKLCAWLQL .SYVHKQS.D SWLKKFTEAC NAARGLEWIG QKISKFIDWI |
| hrv-89-f08 | KEKLCAWLQL .SYVHKQS.D SWLKKFTEAC NAARGLEWIG QKISKFIDWI |
| hrv-89-f09 | KEKLCAWLQL .SYVHKQS.D SWLKKFTEAC NAARGLEWIG QKISKFIDWI |
| m16248-89 | KEKLCAWLQL .SYVHKQS.D SWLKKFTEAC NAARGLEWIG QKISKFIDWI |
| hrv-90 | KEKFCKWTQL .NYVHKES.D SWLKKFTEMC NAARGLEWIG NKISKFIEWM |
| hrv-94 | KEKFCKWTQL .NYVHKES.D SWLKKFTEMC NAARGLEWIG NKISKFIDWM |
| ef173419-94 | KEKFCKWTQL .NYVHKES.D SWLKKFTEMC NAARGLEWIG NKISKFIDWM |
| hrv-95 | KQKVCQWLGL .RYVHKES.D GWIKKFTEMC NAARGLEWIG CKISKFIDWL |
| hrv-96 | KEKFCKWTQL .NYIHKES.D SWLKKFTEMC NAARGLEWIG NKISKFIEWM |
| hrv-98 | KEKFCKWTQL .NYIHKES.D SWLKKFTEMC NAARGLEWIG NKISKFIDWM |
| hrv-100 | KEKFCKWTQL .NYIHKES.D SWLKKFTEMC NAARGLEWIG NKISKFIEWM |
| ef186077-qpm | KDKVCKWLGI DPPPSRQS.D SWLKKFTEFC NAARGLEWIG DKLSKFIDWL |
| ef077279-nat001 | KDKVCKWLGI DPPPSRQG.D SWLKKFTEFC NAARGLEWIG DKLSKFIDWL |
| ny1078 | KDKVCRWLGI DPPPSRQG.D SWLKKFTEFC NAARGLEWIG DKLSKFIDWL |
| ef582385-c024 | KDRLCQWLNI TRPPSRQG.D GWLKKFTECC NAAKGLEWIG IKISKFIEWL |
| ef077280-nat045 | KSKVCKWLGI NPPAKRQG.D SWLKKFTEWC NAAKGLEWVG QKISRFIDWL |
| ef582387-c026 | KDKVCKWLGI DPPPSKQG.D SWLKKFTEFC NAARGLEWIG DKLSKFIDWL |
| ef582386-c025 | KTKLCAWLGV QKPPSKQS.D SWLKKFTEWC NAAKGLEWIG YKISKFIDWL |
| dq473485-03 | KMYISKHFQV .PYIERQAND GWFRKFNDAC NAAKGLEWIA NKISKLIEWI |
| ef173422-03 | KMYISKHFQV .PYIERQAND GWFRKFNDAC NAAKGLEWIA NKISKLIEWI |
| dq473490-04 | KMYISKHFCL .PYIERQSND LWFKKFNDMC NAAKGLEWIA NKISKLIDWV |
| hrv-05 | KMYISKHFCL .PYIERQSND LWFKKFNDMC NAAKGLEWIA NKISKLIEWV |
| dq473486-06 | KMYISKHFQV .PYIERQAND GWFRKFNDAC NAAKGLEWIA NKISKLIGWI |
| l05355-14 | KMYISKHFQV .PYIERQAND GWFRKFNDAC NAAKGLEWIA NKISKLIEWI |
| k02121-14 | KMYISKHFQV .PYIERQAND GWFRKFNDAC NAAKGLEWIA NKISKLIEWI |
| ef173420-17 | KMYISKHFQV .PYIERQAND GWFRKFNDAC NAAKGLEWIA NKISKLIEWV |
| hrv-26 | KMYISKHFCI .PYIERQSND LWFKKFNDMC NAAKGLEWIA NKISKLIEWV |
| hrv-27 | KMYISKHFHL .PYIERQSND LWFKKFNDMC NAAKGLEWIA NKISKLIDWI |
| ef173421-27 | KMYISKHFHL .PYIERQSND LWFKKFNDMC NAAKGLEWIA NKISKLIDWI |
| dq473487-35 | KMYISKHFQV .PYIERQAND GWFRKFNDAC NAAKGLEWIA NKISKLIEWI |
| hrv-35 | KMYISKHFQV .PYIERQAND GWFRKFNDAC NAAKGLEWIA NKISKLIEWI |
| ef173423-37 | KMYISKHFQV .PYIERQAND GWFRKFNDAC NAAKGLEWIA NKISKLIEWI |
| hrv-42 | KMYISKHFCL .PYIERQSND LWFKKFNDMC NAAKGLEWIA NKISKLIDWV |
| dq473488-48 | KMYISKHFQV .PYIERQAND GWFRKFNDAC NAAKGLEWIA NKISKLIEWV |
| hrv-52 | KMYISKHFQV .PYIERQAND GWFRKFNDAC NAAKGLEWIA NKISKLIEWV |
| hrv-52-f10 | KMYISKHFQV .PYIERQAND GWFRKFNDAC NAAKGLEWIA NKISKLIEWV |
| ef173424-52 | KMYISKHFQV .PYIERQAND GWFRKFNDAC NAAKGLEWIA NKISKLIEWV |
| hrv-69 | KMYISKHFQV .PYIERQAND GWFRKFNDAC NAAKGLEWIA NKISKLIEWV |
| dq473489-70 | KMYISKHFQV .PYIERQAND GWFRKFNDAC NAAKGLEWIA NKISKLIEWI |
| hrv-72 | KMYISKHFQV .PYIERQAND GWFRKFNDAC NAAKGLEWIA NKISKLIEWV |
| hrv-79 | KMYISKHFQV .PYIERQAND GWFRKFNDAC NAAKGLEWIA NKISKLIEWI |
| hrv-83 | KMYISKHFQV .PYIERQAND GWFRKFNDAC NAAKGLEWIA NKISKLIEWI |
| hrv-84 | KMYISKHFHL .PYIERQSND LWFKKFNDMC NAAKGLEWIA NKISKLIDWI |
| hrv-86 | KMYVSKHFQV .PYIERQAND GWFRKFNDAC NAAKGLEWIA NKISKFIDWV |
| hrv-91 | KMYISKHFQV .PYIERQAND GWFRKFNDAC NAAKGLEWIA NKISKLIEWI |
| hrv-92 | KMYISKHFQV .PYIERQAND GWFRKFNDAC NAAKGLEWIA NKISKLIEWI |
| ef173425-93 | KMYISKHFHL .PYIERQSND LWFKKFNDMC NAAKGLEWIA NKISKLIEWV |
| hrv-97 | KMYISKHFHL .PYIERQSND LWFKKFNDMC NAAKGLEWIA NKISKLIDWI |
| hrv-99 | KMYISKHFCL .PYIERQSND LWFKKFNDMC NAAKGLEWIA NKISKLIEWV |
| | 1201 1250 |
| annotation | .......... .......... .......... .......... .......... |
| af499637-cva13 | KTKIIPEARD KVEYVTKLKQ LGMLENQINT IHQSCP.SQE QQEILFNNVR |
| af546702-cva21 | KCKIIPDAKD KVEFLTKLKQ LDMLENQIAT IHQSCP.SQE QQEILFNNVR |
| v01149-pv1m | KEKIIPQARD KLEFVTKLRQ LEMLENQIST IHQSCP.SQE HQEILFNNVR |
| d00239-01 | KS.MLPQAQL KVKYLNEIKK LSLLEKQIEN LRAADNATQE K...IKCEID |
| pico-01 | KS.MLPQAQL KVKYLNEIKK LSLLEKQIEN LRAADSATQE K...IKCEID |
| hrv-01 | KS.MLPQAQL KVKYLNEIKK LSLLEKQIEN LRAADSATQE K...IKCEID |
| x02316-02 | KS.MLPQAQL KVKYLNELKK LNLYEKQVES LRVADMKTQE K...IKMEID |
| hrv-07 | KS.MLPQAQL KVEYLNKLKQ LNLLEKQIET IRLAPTCNQE K...IFCDIN |
| dq473503-07 | KS.MLPQAQL KVEYLNKLKQ LNLLEKQIET IRLAPTCNQE K...IFCDIN |
| hrv-08 | KS.MLPQAQN KIKFLQFTKQ LQLKEKQIDG LPYATIKQQE E...YLQEME |
| pico-09 | KS.MLPQAQL KVKYLSELKK LNLLEKQIDN LRCADTKVQE K...IKFEIE |
| hrv-09 | KS.MLPQAQL KVKYLSELKK LNLLEKQIDN LRCADTKVQE K...IKFEID |
| hrv-09-f01 | KS.MLPQAQL KVKYLSELKK LNLLEKQIDN LRCADTKVQE K...IKFEIE |
| hrv-09-f02 | KS.MLPQAQL KVKYLSELKK LNLLEKQIDN LRCADTKVQE K...IKFEIE |
| dq473498-10 | KS.MLPQAQL KVKYLSELKK LSFLEKQMEH LRVADSKTQE K...IKHEVD |
| hrv-10 | KS.MLPQAQL KVKYLSELKK LSFLEKQMEH LRVADSKTQE K...IKHEVD |
| ef173414-11 | KS.MLPQAQL KIKYLNELKK LNLLEKQIEH LRIADSNTQE K...IKCEID |
| ef173415-12 | KA.MLPQTQL KLEYLKLLKQ LPLLEKQIDG LRAATEKQRE D...YTETIS |
| hrv-13 | KS.MLPQAQL KVKYLNELKK LNLLEKQVEN LRCADTKTQE K...IKVEID |
| hrv-13-f03 | KS.MLPQAQL KVKYLNELKK LNLLEKQVEN LRCADTKTQE K...IKVEVD |
| dq473493-15 | KS.MLPQAQL KVKYLSELKK LNLLEKQIEN LRGADTKTQD K...IKFEID |
| l24917-16 | KS.MLPQAQL KVKYLSELKK LNFLEKQVEN LRAADTNTQE K...IKCEID |
| hrv-18 | KS.MLPQAQL KVKYLSELKK LSFLEKQMEH LRVADSKTQE K...IKCEID |
| hrv-19 | KS.MLPQAQL KVKYLSELKK LGFLEKQIEN LRCADTKTQE K...IKLEID |
| hrv-20 | KS.MLPQAKL KVEFIKNLKQ LPLLEKQVDG LRLATQKQQQ D...YIDTLT |
| hrv-21 | KS.MLPQAQL KVKYLSELKK LNLLEKQIEH LRAADSATQE K...IKCEID |
| hrv-22 | KS.MLPQAQL KVKYLSELKK LNLLEKQIEN LRGADSKTQE K...IKLEID |
| dq473497-23 | KS.MLPQAQL KVKYLNELKK LSLYEKQVES LRVADIKTQE K...IKMEID |
| hrv-24 | KS.MLPQAQL KIKYLSELKK LNLLEKQIEH LRIADSSTQE K...IKCEID |
| ef173416-24 | KS.MLPQAQL KIKYLSELKK LNLLEKQIEH LRIADSSTQE K...IKCEID |
| hrv-25 | KS.MLPQAQL KVKYLSELKK LNFLEKQMEH LRVADSQTQE K...IKHEID |
| dq473508-28 | KS.MLPQAKL KMEFIKNLKQ LPLLEKQVDG LRLATQKQQQ D...YIDILT |
| hrv-29 | KS.MLPQAQL KVKYLSELKK LSFLEKQMEH LRVADTQTQE K...IKHEID |
| dq473512-30 | KS.MLPQAQL KVKYLNELKK LSLYEKQVES LRVADIKTQE K...IKMEID |
| hrv-30 | KS.MLPQAQL KVKYLNELKK LSLYEKQVES LRVADIKTQE K...IKMEID |
| hrv-31 | KS.MLPQAQL KIKYLSELKK LSFLEKQIEY LRVADSQTQE K...IKCEID |
| hrv-32 | KS.MLPQAQL KVKYLSELKK LNLLEKQIDN LRCADTKVQE K...IKFEID |
| hrv-33 | KS.MLPQAQL KIKYLNELKK LNLLEKQIEH LRIADSSTQE K...IKCEID |
| hrv-34 | KS.MLPQAQL KVKYLNELKK LSFLEKQMEH LRVADSQTQE K...IKCEID |
| dq473501-34 | KS.MLPQAQL KVKYLNELKK LSFLEKQMEH LRVADSQTQE K...IKCEID |
| dq473505-36 | KS.MLPQAQL KVDYLTKLKQ LNLLEKQIET ITLAPANDQE K...IFTEIN |
| hrv-38 | KS.MLPQAQL KVKYLSELKK LNLLEKQIDN LRCADSKVQE K...IKLEID |
| dq473495-38 | KS.MLPQAQL KVKYLSELKK LNLLEKQIDN LRCADSKVQE K...IKLEID |
| ay751783-39 | KS.MLPQAQL KVKYLNELKR LNLYEKQVEN LRVADIKTQE K...IKMEID |
| hrv-40 | KS.MLPQAQL KVKYLSELKK LSFLEKQMEH LRVADSQTQE K...IKHEID |
| dq473491-41 | RS.MLPQAQL KVKYLSELKK LNLLEKQVEN LRCADSKTQE K...IKIEVD |
| hrv-43 | KS.MLPQAQL KVKYLAELKK LNLLEKQIEN LRGADTNTQE K...IKLEID |
| dq473499-44 | KS.MLPQAQL KVKYLSELKK LSFLEKQIEH LRVADTQTQE K...IKHEID |
| hrv-45 | KS.MLPQAQN KIKFLHFMKQ LQLKEKQIDG LPYATVKQQE D...YLKEME |
| dq473506-46 | KS.MLPQARL KVDFIKNLKQ LPLLEKQVDG LRLATQKQQQ E...YIDTLT |
| hrv-47 | KS.MLPQAQL KVKYLSELKK LSFLEKQIEH LRVADSQTQE K...IKCEID |
| dq473496-49 | KS.MLPQAQL KVKYLNELKK LSLYEKQVES LRVADVRTQE K...IKMEID |
| hrv-49-f04 | KS.MLPQAQL KVKYLNELKK LSLYEKQVES LRIADVRTQE K...IKMEID |
| hrv-50 | KS.MLPQAQL KIKYLSELKK LNFLEKQMEH LRVADSQIQE K...IKHEVD |
| hrv-51 | KS.MLPQAKL KVEYIKNLKQ LPLLEKEIDG LRLASQKQQQ E...YIDTLR |
| dq473507-53 | KS.MLPQARL KVDFIKNLKQ LPLLEKQVDG LRLATQKQQQ E...YIDTLT |
| hrv-54 | KS.MLPQAQL KIKYLSELKK LNFLEKQMEH LRVADSQTQE K...IKHEID |
| hrv-54-f05 | KS.MLPQAQL KIKYLSELKK LNLLEKQMEH LRVADSQTQE K...IKHEID |
| dq473511-55 | KS.MLPQAQL KVKYLSELKK LNLLEKQIEH LRAADSATQE K...IKCEID |
| hrv-56 | KS.MLPQAQL KIKYLSELKK LNFLEKQMEH LRIADSQTQE K...IKHEVD |
| hrv-57 | KS.MLPQAQL KIKYLSELKK LNLLEKQIEH LRVADSATQE K...IKCEID |
| hrv-58 | KG.MLPQAQL KVDYLTKLKQ LNLLEKQIET IRLAPANDQE K...IFTEIS |
| dq473500-59 | KS.MLPQAQL KVKYLEGLKK LNFLEKQIEH LRVADSQTQE K...IKCEID |
| hrv-60 | KS.MLPQAQL KVKYLSELKK LNLLEKQIEN LRGADTNTQE K...IKLEID |
| hrv-61 | KS.MLPQAQL KVKYLNELKK LNLLEKQIEN LRSADSKTQE R...IKVEID |
| hrv-62 | KS.MLPQAQL KVKYLSELKK LSFLEKQMEH LRVADSQTQE K...IKHEID |
| hrv-63 | KS.MLPQAQL KVKYLKELKK LNFLEKQIEH LRVADSQTQE K...IKCEID |
| hrv-64 | KS.MLPQAQL KVKYLSELKK LNLLEKQIEN LRGADTKTQE K...IKLEID |
| ef173417-64 | KS.MLPQAQL KVKYLSELKK LNLLEKQIEN LRGADTKTQE K...IKLEID |
| hrv-65 | KS.MLPQAKL KVEYIKNLKQ LPLLEKEIDG LRLASQKQQQ D...YIDTLK |
| hrv-66 | KS.MLPQAQL KVKYLSELKK LNFLEKQIEH LRVADSKTQE K...IKHEID |
| hrv-67 | KS.MLPQAQL KVKYLSELKK LNLLEKQIDN LRCADTKVQE K...IKFEID |
| hrv-68 | KS.MLPQAKL KVEFIKNLKQ LPLLEKQVDG LRLATQKQQQ D...YVDTLT |
| hrv-71 | KS.MLPQAKL KVEYIKNLKQ LPLLEKQIDG LRLAPQKQQQ E...YIDTLR |
| dq473492-73 | KS.MLPQAQL KVKYLNELKK LSLLEKQVEN LRCADTKTQE K...IKVEID |
| dq473494-74 | KS.MLPQAQL KVKYLSELKK LNLLEKQIEN LRSADTKTQE K...IKLEID |
| dq473510-75 | KS.MLPQAQL KVKYLSELRK LNLLEKQIEN LRAADANTQE K...IKLEID |
| dq473502-76 | KS.MLPQAQL KIKYLNELKK LNLLEKQIEH LRIADSSTQG K...IKCEID |
| hrv-76 | KS.MLPQAQL KIKYLNELKK LNLLEKQIEH LRIADSSTQE K...IKCEID |
| hrv-77 | KS.MLPQAQL KVKYLSELKK LNFLEKQIEY LRVADSRTQE K...IKHEID |
| hrv-78 | KS.MLPQAKL KVTYIQSLKQ LPLLEKSVDG LRLANPEKQY L...FLKQIE |
| ef173418-78 | KS.MLPQAKL KVTYIQSLKQ LPLLEKSVDG LRLANPEKQY L...FLKQIE |
| hrv-80 | KS.MLPQAKL KVEFIKMLKQ LPLLEKEIDG LRLASQKQQQ E...YVETLR |
| hrv-81 | KS.MLPQAQL KVKYLNELKK LNLLEKQIEN LRAADANSQE K...IKCEID |
| hrv-81-f06 | KS.MLPQAQL KVKYLNELKK LNLLEKQIEN LRAADTNTQE K...IKCEID |
| hrv-81-f07 | KS.MLPQAQL KVKYLNELKK LNLLEKQIEN LRAADTNTQE K...IKCEID |
| hrv-82 | KS.MLPQAQL KVKYLNELKK LSLLEKQIEN LRCADTKTQE K...IKVEVD |
| dq473509-82 | KS.MLPQAQL KVKYLNELKK LSLLEKQIEN LRCADAKTQE K...IKVEVD |
| hrv-85 | KS.MLPQAQL KVKYLSELKK LNLLEKQIEH LRVADSHTQE K...IKHEID |
| pico-85 | KS.MLPQAQL KVKYLSELKK LNLLEKQIEH LRVADSHTQE K...IKHEID |
| dq473504-88 | KS.MLPQAQL KVDYLTKLKQ LNLLEKQIES IRLAPSCDQE K...IFNEIN |
| a10937-89 | KS.MLPQAQL KIDYLTKLKQ LNLLEKQIET IRLAPASVQE K...IFIEIN |
| hrv-89 | KS.MLPQAQL KIDYLTKLKQ LNLLEKQIET IRLAPASVQE K...IFIEIN |
| hrv-89-f08 | KS.MLPQAQL KIDYLTKLKQ LNLLEKQIET IRLAPASDQE K...IYIEIN |
| hrv-89-f09 | KS.MLPQAQL KIDYLTKLKQ LNLLEKQIET IRLAPASDQE K...IYIEIN |
| m16248-89 | KS.MLPQAQL KIDYLTKLKQ LNLLEKQIET IRLAPASVQE K...IFIEIN |
| hrv-90 | KS.MLPQAQL KIKYLAELKK LNFLEKQIEH LRVADSKTQE K...IKCEVD |
| hrv-94 | KS.MLPQAQL KVKYLSELKK LNLLEKQIEN LRGADTRTQE K...IKLEID |
| ef173419-94 | KS.MLPQAQL KVKYLSELKK LNLLEKQIEN LRGADTRTQE K...IKLEID |
| hrv-95 | KS.MLPQAQN KIKFLQFTKQ LQLKEKQIDG LPYATIKQQE E...YLQEME |
| hrv-96 | KS.MLPQAQL KVKYLNELKK LNLLEKQIEN LRSADSKTQE R...IKIEID |
| hrv-98 | KS.MLPQAQL KIKYLSELKK LSFLEKQMEH LRVADSQTQE K...IKHEID |
| hrv-100 | KS.MLPQAQL KVKYLSELKK LSFLEKQMEH LRVADTQTQE K...IKHEID |
| ef186077-qpm | KGKILPTLRR KSDTLKECKK IPLYKEQVKA FATASEEAQD E...LMVKVN |
| ef077279-nat001 | KGKILPTLRR KSDTLKECKK IPLYKEQVKA FATASEEAQD E...LLVKVS |
| ny1078 | KGKILPTLRR KSETLKECKK IPLYKEQVRA FATASEDAQD E...LMVNIS |
| ef582385-c024 | KNRLLPTLQH KKEILDECKK IDLYQEQVKG FSKVDEPAQQ E...LLIKVE |
| ef077280-nat045 | KERVLPTVKS KKEILDHCKK IPLYQEQIKG FPYATQESQE E...LALNID |
| ef582387-c026 | KGKILPTLRR KSDTLKECKK IPLYKEQVKA FATASEEAQD E...LIVKVN |
| ef582386-c025 | KEKLIPTVQR KKDTLLECKK LTLYEDQVKA FPQSSEIFQN E...LTTKLQ |
| dq473485-03 | KNKVLPQARE KLEFCSKLKQ LDILERQIAS IHDSNP.TQE KREQLFNNVL |
| ef173422-03 | KNKVLPQARE KLEFCSKLKQ LDILERQIAS IHDSNP.TQE KREQLFNNVL |
| dq473490-04 | KQKILPQARD KLDFCKRLKQ LDILERQIDT LHVNQV.NDN DRETLFNNVL |
| hrv-05 | KQKILPQARD KLDFCRRLKQ LDILERQIDT LHMNQV.NDN DREVLFNNIL |
| dq473486-06 | KNKVLPQAKE KLEYCSKLKQ LDILERQITT MHVSNP.SQE KREQLFNNVL |
| l05355-14 | KNKVLPQAKE KLEFCSKLKQ LDILERQITT MHISNP.TQE KREQLFNNVL |
| k02121-14 | KNKVLPQAKE KLEFCSKLKQ LDILERQITT MHISNP.TQE KREQLFNNVL |
| ef173420-17 | KTKIIPQAKE KLDFCKRLKQ LDILERQIAT MHVSNP.TQE KREQLFNNVL |
| hrv-26 | KLKVLPQARD KLDFCKRLKQ LDILERQINT LHMNQI.SDN DREVLFNNVL |
| hrv-27 | KNKILPQARD KLDFCKRLKQ LDILERQIDT LHMNYT.NEN DRETLFNNVL |
| ef173421-27 | KNKILPQARD KLDFCKRLKQ LDILERQIDT LHMNYT.NEN DRETLFNNVL |
| dq473487-35 | KNKVLPQARE KLEFCSKLKQ LDILERQITT MHISNP.TQE KRKYLFNNVM |
| hrv-35 | KNKVLPQARE KLEFCSKLKQ LDILERQITT MHISNP.TQE KREQLFNNVM |
| ef173423-37 | KNKVLPQAKE KLEYCSKLKQ LDILERQITT MHVSNP.TQE KREQLFNNVL |
| hrv-42 | KQKILPQARD KLDFCRRLKQ LDILERQIDS LHMNQV.NDN DREVLFNNIL |
| dq473488-48 | KTKIIPQAKE KLDFCRRLKQ LDILERQIAT MHVSNP.TQE KREQLLNNVM |
| hrv-52 | KTKIIPQARE KLDFCKRLKQ LDILERQIAT MHVSNP.TQE KREQLFNNVL |
| hrv-52-f10 | KTKIIPQAKE KLDFCKRLKQ LDILERQITT MHVSNP.TQE KREQLFNNVL |
| ef173424-52 | KTKIIPQARE KLDFCKRLKQ LDILERQIAT MHVSNP.TQE KREQLFNNVL |
| hrv-69 | KTKIIPQAKE KLDFCKRLKQ LDILERQVTT MHVSNP.TQE KREQLFNNIL |
| dq473489-70 | KVKIIPQAKE KLDFCKRLKQ LDILERQIAT MHVSNP.TQE KREQLFNNVL |
| hrv-72 | KNKVLPQAKE KLEFCSKLKQ LDLLERQINT MHVSNP.TQE KREQLFNNVL |
| hrv-79 | KNKVLPQAKE KLEFCSKLKQ LDILERQITT MHVSNP.TQE KREQLFNNVM |
| hrv-83 | KNKVLPQAKE KLEFCSKLKQ LDILERQITT MHVSNP.TQE KREQLFNNVM |
| hrv-84 | KNKLLPQAKD KLDFCKRLKQ LDIIERQVET LHMNHT.NEN DREVLFNNIL |
| hrv-86 | KTKILPQARE KLDFCRRLKQ LDILERQITT MHISNP.TQE KREQLFNNIL |
| hrv-91 | KTKIIPQAKE KLDFCKRLKQ LDIMERQIAT MHVSNP.TQE KREQLFNNVL |
| hrv-92 | KNKVLPQAKE KLDFCSKLKQ LDMLERQITT MHVSNP.TQE KREQLFNNVM |
| ef173425-93 | KNKILPQARD KLDFCKRLKQ LDILERQIDT LHMNHT.NEN DREVLFNNVL |
| hrv-97 | KNKILPQARD KLDFCKRLKQ LDILERQIDT LHMNQT.NEN DRETLFNNVL |
| hrv-99 | KQKILPQARD KLDFCKRLKQ LDVLERQIDT LHMNQV.SDN DRETLFNNIL |
| | 1251 1300 |
| annotation | .......... .......... .......... .......... .......... |
| af499637-cva13 | WLAIQSRRFA PLYAVEAKRI SKLENTINNY IQFKSKHRIE PVCVLIHGSP |
| af546702-cva21 | WLAVQSRRFA PLYAVEARRI NKMESTINNY IQFKSKHRIE PVCMLIHGSP |
| v01149-pv1m | WLSIQSKRFA PLYAVEAKRI QKLEHTINNY IQFKSKHRIE PVCLLVHGSP |
| d00239-01 | TLHDLSCKFL PLYAHEAKRI KVLYNKCSNI I..KQRKRSE PVAVMIHGPP |
| pico-01 | TLHDLSCKFL PLYAHEAKRI KVLYNKCSNI I..KQRKRSE PVAVMIHGPP |
| hrv-01 | TLHDLSCKFL PLYAHEAKRI KVLYNKCSNI I..KQRKRSE PVAVMIHGPP |
| x02316-02 | TLHDLSRKFL PLYASEAKRI KTLYIKCDNI I..KQKKRCE PVAIVIHGPP |
| hrv-07 | TLHDLSLKFL PLYASEARRI KNLYNKCNNV I..KGGKRNE PVAVLIHGSP |
| dq473503-07 | TLHDLSLKFL PLYASEARRI KNLYNKCNNV I..KGGKRNE PVAVLIHGSP |
| hrv-08 | EMLDISNKLL PLYPRENKMI KDLLKQAKNM T..VASKRIE PVAVMFHGDP |
| pico-09 | TLHDLSCKFL PLYASEAKRI KILHNKCCNI I..KQRKRSE PVAIMIHGPP |
| hrv-09 | TLHDLSCKFL PLYASEAKRI KILHNKCCNI I..KQRKRSE PVAIMIHGPP |
| hrv-09-f01 | TLHDLSCKFL PLYASEAKRI KILHNKCCNI I..KQRKRSE PVAIMIHGPP |
| hrv-09-f02 | TLHDLSCKFL PLYASEAKRI KILHNKCCNI I..KQRKRSE PVAIMIHGPP |
| dq473498-10 | TLHDLSCKFL PLYASEAKRI KILHNKCNTI I..KQRKRCE PVAVVIHGEP |
| hrv-10 | TLHDLSCKFL PLYASEAKRI KILHNKCNTI I..KQRKRCE PVAVVIHGEP |
| ef173414-11 | TLHDLSCKFL PLYASEAKRI KVLHNKCNVI I..KQKKRCE PVAVVIHGEP |
| ef173415-12 | RLDDLSKKFL PLYPSEAKRI SVLYKRSQTI L..KTSKRTE PVGILFHGEP |
| hrv-13 | TLHDLSCKFL PLYASEAKRI KVIYNKCNNI I..KQRKRSE PVAVMIHGLP |
| hrv-13-f03 | TLHDLSCKFL PLYASEAKRI KVIYNKCSNI I..KQRKRSE PVAVMIHGSP |
| dq473493-15 | TLHDLSCKFL PLYASEAKRI KALHNKCNII I..KQKKRSE PVAIMIHGPP |
| 124917-16 | TLHDLSCKFL PLYASEAKRI KVLYHKCTNI I..KQKKRSE PVAVMIHGPP |
| hrv-18 | TLHDLSCKFL PLYASEAKRI KILHNKCNTI I..KQKKRCE PVAVVIHGEP |
| hrv-19 | TLHDLSCKFL PLYASEAKRI KVLYNKCNNI I..KQKRRSE PVAIMIHGPP |
| hrv-20 | LMLDSSNKFL PLYAVENKRI KELLKRSQMI L..RTSKRTE PVGVIFHGEP |
| hrv-21 | TLHDLSCKFL PLYASEAKRI KVLHNKCNVV I..KQKKRCE PVAVVIHGEP |
| hrv-22 | TLHDLSCKFL PLYASEAKRI KVLXNKCNNV I..KQKKRSE PVAVMIHGPP |
| dq473497-23 | TLHDLSCKFL PLYASEAKRI KTLHIKCDNI I..KQKKRCE PVAIVIHGPP |
| hrv-24 | TLHDLSCKFL PLYASEAKRI KILYNKCNVI I..KQKKRCE PVAVVIHGEP |
| ef173416-24 | TLHDLSCKFL PLYASEAKRI KILYNKCNVI I..KQKKRCE PVAVVIHGEP |
| hrv-25 | TLHDLSCKFL PLYASEAKRI KILHNKCSTI I..KQRKRCE PVAVVIHGEP |
| dq473508-28 | LMLDSSNKFL PLYAVENKRI KELLKRSQMI L..RTSKRTE PVGVIFHGEP |
| hrv-29 | TLHDLSCKFL PLYASEAKRI KILHNKCSTI I..KQRKRCE PVAVVIHGDP |
| dq473512-30 | TLHDLSCKFL PLYASEAKRI KNLHIKCNNI I..KQKKRCE PVAIVIHGPP |
| hrv-30 | TLHDLSCKFL PLYASEAKRI KNLHIKCNNI I..KQKKRCE PVAIVIHGPP |
| hrv-31 | TLHDLSCKFL PLYASEAKRI KILHNKCNTI I..KQRKRCE PVAVAIHGEP |
| hrv-32 | TLHDLSCKFL PLYASEAKRI KTLHNKCCNI I..KQKKRSE PVAIMIHGPP |
| hrv-33 | TLHDLSCKFL PLYASEAKRI KVLYNKCNVI I..KQKKRCE PVAVVIHGEP |
| hrv-34 | TLHDLSCKFL PLYASEAKRI KILHNRCSTI I..KQRKRCE PVAVVIHGEP |
| dq473501-34 | TLHDLSCKFL PLYASEAKRI KILHNRCSTI I..KQRKRCE PVAVVIHGEP |
| dq473505-36 | TLHDLSLKFL PLYASEARRI KSLYIKCSNI I..KGGKRNE PVAVLIHGSP |
| hrv-38 | TLHDLSCKFL PLYASEAKRI KVLFNKCNTI I..KQKKRSE PVAIMVHGPP |
| dq473495-38 | TLHDLSCKFL PLYASEAKRI KVLFNKCNTI I..KQKKRSE PVAIMVHGPP |
| ay751783-39 | TLHDLSCKFL PLYASEAKRI KILHNKCDTI I..KQKKRSE PVAIVVHGPP |
| hrv-40 | TLHDLSCKFL PLYASEAKRI KVLHNKCSAI I..KQKKRCE PVAVVIHGEP |
| dq473491-41 | TLHDLSCKFL PLYASEAKRI KVVYNKCNNI I..KQKKRSE PVAVMIHGSP |
| hrv-43 | TLHDLSCKFL PLYASEAKRI KVLHNKCNNI I..KQRKRSE PVAIMIHGPP |
| dq473499-44 | TLHDLSCKFL PLYASEAKRI KILHNKCSTI I..KQRKRCE PVAVVIHGDP |
| hrv-45 | EMLDISNKLL PLYPKENKII KDLLKQAKSM T..TTSKRVE PVAIMFHGDP |
| dq473506-46 | LMLDSSNKFL PLYALENKRI KELLKRGQMI L..RTSKRTE PVGVIFHGEP |
| hrv-47 | TLHDLSCKFL PLYASEAKRI KILHNKCNTI I..KQKKRCE PVAVAIHGEP |
| dq473496-49 | TLHDLSCKFL PLYAGEAKRI KTLHIKCDNI I..KQKKRCE PVAIVIHGPP |
| hrv-49-f04 | TLHDLSCKFL PLYASEAKRI KTLHIKCDNI I..KQKKRCE PVAVVIHGEP |
| hrv-50 | TLHDLSCKFL PLYASEAKRI KVLYNKCNTI I..KQKKRCE PVAVVIHGEP |
| hrv-51 | IMLDSSNKFL PLYASENKRI KELLKRSEMI L..RTSKRTE PVGVLFHGEP |
| dq473507-53 | LMLDSSNKFL PLYALENKRI KELLKRGQMI L..RTSKRTE PVGVIFHGEP |
| hrv-54 | TLHDLSCKFL PLYASEAKRI KVLYNRCSTI I..KQKKRCE PVAVVIHGEP |
| hrv-54-f05 | TLHDLSCKFL PLYASEAKRI KILHNRCSTI I..KQKKRCE PVAVVIHGEP |
| dq473511-55 | TLHDLSCKFL PLYASEAKRI KVLHNKCNVV I..KQRKRCE PVAVVIHGEP |
| hrv-56 | TLHDLSCKFL PLYASEAKRI KILYNKCNTI I..KQRKRCE PVAVVIHGEP |
| hrv-57 | TLHDLSCKFL PLYASEAKRI KVLYNKCNVV I..KQKKRCE PVAVVIHGEP |
| hrv-58 | TLHDLSLKFL PLYASEARRI KNLYIKCNNI I..KGGKRNE PVAVLIHGSP |
| dq473500-59 | TLHDLSCKFL PLYASEAKRI KVLHNKCNVI I..KQKKRCE PVAVVIHGEP |
| hrv-60 | TLHDLSCKFL PLYASEAKRI KVLFNKCNTI I..KQKKRSE PVAIMIHGPP |
| hrv-61 | TLHDLSCKFL PLYASEAKRI KVIHNKCNTI I..KQKKRSE PVAVMIHGSP |
| hrv-62 | TLHDLSCKFL PLYASEAKRI KILHNKCSTI I..KQRKRCE PVAVVIHGEP |
| hrv-63 | TLHDLSCKFL PLYASEAKRI KVLHNKCNII I..KQKKRCE PVAVVIHGEP |
| hrv-64 | TLHDLSCKFL PLYASEAKRI KVLYNKCNNV I..KQKKRSE PVAVMIHGPP |
| ef173417-64 | TLHDLSCKFL PLYASEAKRI KVLYNKCNNV I..KQKKRSE PVAVMIHGPP |
| hrv-65 | IMLDSSNKFL PLYACENKRI KELLKRSEMI L..RTSKRTE PVGVLFHGEP |
| hrv-66 | TLHDLSCKFL PLYASEAKRI KILHNRCSTI I..KQKKRCE PVAVVIHGEP |
| hrv-67 | TLHDLSCKFL PLYASEAKRI KILHNKCCNI I..KQKKRSE PVAIMIHGPP |
| hrv-68 | LMLDSSNKFL PLYATENKRI KELLKRSQMI L..RTSKRTE PVGVIFHGEP |
| hrv-71 | IMLDSSNKFL PLYASENKRI KELLKRSDMI L..RTSKRTE PVGVLFHGDP |
| dq473492-73 | TLHDLSCKFL PLYASEAKRI KVIYNKCNNI I..KQKKRSE PVAVMIHGSP |
| dq473494-74 | TLHDLSCKFL PLYASEAKRI KALHNKCNIV I..KQKKRSE PVAVMIHGPP |
| dq473510-75 | TLHDLSCKFL PLYASEAKRI KALYNKCNNI I..KQRKRSE PVAIMIHGPP |
| dq473502-76 | TLHDLSCKFL PLYASEAKRI KVLHNKCNVI I..KQKKRCE PVAVVIHGEP |
| hrv-76 | TLHDLSCKFL PLYASEAKRI KVLHNKCNVI I..KQKKRCE PVAVVIHGEP |
| hrv-77 | TLHDLSCKFL PLYASEAKRI KILHNRCNTI I..KQKKRCE PVAVVIHGEP |
| hrv-78 | EMKDLSNKFL PLYANEAKRI KALAHKASNI V..KNAKRVE PVAVMFHGNP |
| ef173418-78 | EMKDLSNKFL PLYANEAKRI KALAHKASNI V..KNAKRVE PVAVMFHGNP |
| hrv-80 | IMADSSRKFL PLYASENKRI NDLLRRSEMV M..LTAKRTE PVGVLFHGEP |
| hrv-81 | TLHDLSCKFL PLYASEAKRI KVLYHKCTNI I..KQKKRSE PVAVMIHGPP |
| hrv-81-f06 | TLHDLSCKFL PLYASEAKRI KVLYHKCTNI I..KQKKRSE PVAVMIHGPP |
| hrv-81-f07 | TLHDLSCKFL PLYASEAKRI KVLYHKCTNI I..KQKKRSE PVAVMIHGPP |
| hrv-82 | TLHDLSCKFL PLYASEAKRI KVLYNKCNNI I..KQKKRSE PVAVMIHGPP |
| dq473509-82 | TLHDLSCKFL PLYASEAKRI KVLYNKCNNI I..KQKKRSE PVAVMIHGPP |
| hrv-85 | TLHDLSCKFL PLYASEAKRI KILHNKCSAI I..KQKKRCE PVAVVIHGEP |
| pico-85 | TLHDLSCKFL PLYASEAKRI KILHNKCSAI I..KQKKRCE PVAVVIHGEP |
| dq473504-88 | TLHDLSLKFL PLYASEARRI KNLYIKCNNI I..KGGKRNE PVAVLIHGSP |
| a10937-89 | TLHDLSLKFL PLYASEARRI KNLYIKCSNV I..KGGKRNE PVAVLIHGSP |
| hrv-89 | TLHDLSLKFL PLYASEARRI KNLYIKCSNV I..KGGKRNE PVAVLIHGSP |
| hrv-89-f08 | TLHDLSLKFL PLYASEARRI KNLYIKCSNV I..KGGKRNE PVAVLIHGSP |
| hrv-89-f09 | TLHDLSLKFL PLYASEARRI KNLYIKCSNV I..KGGKRNE PVAVLIHGSP |
| m16248-89 | TLHDLSLKFL PLYASEARRI KNLYIKCSNV I..KGGKRNE PVAVLIHGSP |
| hrv-90 | TLHDLSCKFL PLYASEAKRI KALHNKCNVI I..KQKKRCE PVAVVIHGEP |
| hrv-94 | TLHDLSCKFL PLYASEAKRI KVLYNKCNNI I..KQKKRSE PVAVMIHGPP |
| ef173419-94 | TLHDLSCKFL PLYASEAKRI KVLYNKCNNI I..KQKKRSE PVAVMIHGPP |
| hrv-95 | EMLDISNKLL PLYPRENKMI KDLLKQAKNM T..VASKRIE PVAVMFHGDP |
| hrv-96 | TLHDLSCKFL PLYASEAKRI KVIHNKCNNI I..KQKKRSE PVAVMIHGSP |
| hrv-98 | TLHDLSCKFL PLYASEAKRI KILHNRCSTI I..RQKKRCE PVAVVIHGEP |
| hrv-100 | TLHDLSCKFL PLYASEAKRI KILHNKCNAI I..KQRKRCE PVAVVIHGEP |
| ef186077-qpm | KLEKGLQDLA PLYALEAKQV REMSRELQRM MCYKKTHRTE PVCILLHGQP |
| ef077279-nat001 | KLEKGLQDLA PLYACEAKQV REMSRELQRM MCYKKTHRTE PVCILLHGQP |
| ny1078 | KLEKGLQDLA PLYACEAKQV REMSRDLQRM MCYKKTHRTE PVCILLHGQP |
| ef582385-c024 | KLKKGLDSLA PLYAVENKRV TNIYKELKAL SAYKTTQRTE PVCILIRGAP |
| ef077280-nat045 | KLKKGLDGLA PLYAVENKTV TQMQKEIKQL QCYKRTQRIE PVCILLHGIP |
| ef582387-c026 | KLEKGLQDLA PLYACEAKQV REMSRELQRM MCYKKTHRTE PVCILLHGQP |
| ef582386-c025 | VLKKNLDDMC PLYAVENKRV TNMLKEIKTM TAYKKTHRTE PVCILIHGGP |
| dq473485-03 | WLEQMSQKFS PLYASEAKRI RDLKNKITNY MQFKSKQRTE PVCVLIHGTP |
| ef173422-03 | WLEQMSQKFS PLYASEAKRI RDLKNKITNY MQFKSKQRTE PVCVLIHGTP |
| dq473490-04 | WLEQMSQKFA PLYAVEAKRI KSLKNKITNF IQFRSKHRTE PVCVLIHGTP |
| hrv-05 | WLEQMSQKFA PLYAVEAKRI KTLKNKMTNF IQFRSKHRTE PVCVLVHGSP |
| dq473486-06 | WLEQMSQKFA PLYAAESKRI KDLKNKIMNY MQFKSKQRTE PVCVLIHGTP |
| 105355-14 | WLEQMSQKFA PHYAVESKRI RELKNKMVNY MQFKSKQRTE PVCVLIHGTP |
| k02121-14 | WLEQMSQKFA PFYAVESKRI RELKNKMVNY MQFKSKQRTE PVCVLIHGTP |
| ef173420-17 | WLEQMSQKFA PLYAVEARRI KELKTKIVNY MQFKSKQRTE PVCVLIHGSP |
| hrv-26 | WLEQMSQKFA PLYAVESKRI RTLKNKMTNF IQFRSKHRTE PVCVLIHGTP |
| hrv-27 | WLEQMSQKFA PLYALEAKRI KSLKNKLTNF IQFRSKHRTE PVCVLIHGTP |
| ef173421-27 | WLEQMSQKFA PLYALEAKRI KSLKNKLTNF IQFRSKHRTE PVCVLIHGTP |
| dq473487-35 | WLEQMSQKFA PLYATEAKRI RDLKNKIVNY MQFRNKQRTE PVCVLIHGTP |
| hrv-35 | WLEQMSQKFA PLYATEAKRI RDLKNKIVNY MQFRNKQRTE PVCVLIHGTP |
| ef173423-37 | WLEQMSQKFA PLYAAESKRI RDLKNKIMNY MQFKNKQRTE PVCVLIHGTP |
| hrv-42 | WLEQMSQKFA PLYAVEAKRI KMLKNKMTNF IQFRSKHRTE PVCVLIHGSP |
| dq473488-48 | WLEQMSQKFA PLYAVEARRI KEMKTKMMNY IQFKSKQRTE PVCVLIHGSP |
| hrv-52 | WLEQMSQKFA PLYAVEARRI KEMKTKVMNY IQFKSKQRTE PVCVLIHGSP |
| hrv-52-f10 | WLEQMSQKFA PLYAVEARRI KEMKTKMMNY IQFKSKQRTE PVCVLIHGSP |
| ef173424-52 | WLEQMSQKFA PLYAVEARRI KEMKTKVMNY IQFKSKQRTE PVCVLIHGSP |
| hrv-69 | WLEQMSQKFA PLYAVEARRX KEMKMKMMNY IQFKSKHRTE PVCVLIHGSP |
| dq473489-70 | WLEQMSQKFA PLYAVEARRI KELKTKIVNY MQFKSKQRTE PVCVLIHGSP |
| hrv-72 | WLEQMSQKFA PLYATEAKRI RELKNKIVNY MQFRNKQRTE PVCVLIHGTP |
| hrv-79 | WLEQMSQKFA PLYAAEAKRI RELKNKILNY MQFRSKQRTE PVCVLIHGTP |
| hrv-83 | WLEQMSQKFA PLYAAEARRI KDLKNKILNY MQFKNKQRTE PVCVLIHGTP |
| hrv-84 | WLEQMSQKFA PLYAAEAKRI KTLKNKMTNF IQFRSKHRTE PVCVLIHGSP |
| hrv-86 | WLEQMSQKFA PLYATEGKRI RDLKNKMLNY IQFKSKQRTE PVCVLIHGTP |
| hrv-91 | WLEQMSQKFA PLYAVEARRI KELKTKMVNY MQFKSKQRTE PVCVLIHGSP |
| hrv-92 | WLEQMSQKFA PLYAAEAKRI RELKNKILNY MQFKNKQRTE PVCVLIHGTP |
| ef173425-93 | WLEQMSQKFA PLYALEAKRI RTLKNKMTNF IQFRSKHRTD PVRVLIHGTP |
| hrv-97 | WLEQMSQKFA PLYALEAKRI RSLKNKITNF IQFRSKHRTE PVCVLIHGTP |
| hrv-99 | WLEQMSQKFA PLYAVEAKRI RVLKNKMTNF IQFRSKHRTE PVCVLIHGTP |
| | 1301 1350 |
| annotation | .......... .......... .......... .......... .......... |
| af499637-cva13 | GTGKSVATSL MGRAIAEKEG TSTYSLPPDP THFDGYKQQG VVIMDDLNQN |
| af546702-cva21 | GTGKSIATSL IGRAIAEKES TSVYSMPPDP SHFDGYKQQG VVIMDDLNQN |
| v01149-pv1m | GTGKSVATNL IARAIAEREN TSTYSLPPDP SHFDGYKQQG VVIMDDLNQN |
| d00239-01 | GTGKSITTNF LARMITN..E SDVYSLPPDP KYFDGYDNQS VVIMDDIMQN |
| pico-01 | GTGKSITTNF LARMITN..E SDVYSLPPDP KYFDGYDNQS VVIMDDIMQN |
| hrv-01 | GTGKSITTNF LARMITN..E SDVYSLPPDP KYFDGYDNQS VVIMDDIMQN |
| x02316-02 | GAGKSITTNF LAKMITN..D SDIYSLPPDP KYFDGYDQQS VVIMDDIMQN |
| hrv-07 | GTGKSLATSV LARMLTV..E TDIYSLPPDP KYFDGYDQQS VVIMDDIMQN |
| dq473503-07 | GTGKSLATSV LARMLTV..E TDIYSLPPDP KYFDGYDQQS VVIMDDIMQN |
| hrv-08 | GSGKSICTNI LARMITN..P SDIYSLPPDP KYFDGYHQQT VVIMDDVMQN |
| pico-09 | GTGKPITTSF LARMITN..E SDIYSLPPDP KYFDGYDQQS VVIMDDIMQN |
| hrv-09 | GTGKSITTSF LARMITN..E SDIYSLPPDP KYFDGYDQQS VVIMDDIMQN |
| hrv-09-f01 | GTGKSITTSF LARMITN..E SDIYSLPPDP KYFDGYDQQS VVIMDDIMQN |
| hrv-09-f02 | GTGKSITTSF LARMITN..E SDIYSLPPDP KYFDGYDQQS VVIMDDIMQN |
| dq473498-10 | GTGKSMTTNF LARMITS..E SDIYSLPPDP KYFDGYDQQS VVIMDDVMQN |
| hrv-10 | GTGKSMTTNF LARMITS..E SDIYSLPPDP KYFDGYDQQS VVIMDDVMQN |
| ef173414-11 | GTGKSMTTNF LARMITN..D SDIYSLPPDP KYFDGYDQQS VVIMDDIMQN |
| ef173415-12 | GTGKSITTSV LARMLTS..D SDIYSLPPAP KYFD.VRPQT GVIMDDIMQN |
| hrv-13 | GTGKSITTSF LARMITN..E SDIYSLPPDP KYFDGYDQQS VVIMDDIMQN |
| hrv-13-f03 | GTGKSITTSF LARMITN..E SDIYSLPPDP KYFDGYDQQS VVIMDDIMQN |
| dq473493-15 | GTGKSITTSF LARMITS..E SDIYSLPPDP KYFDGYDQQS VVIMDDIMQN |
| 124917-16 | GTGKSITTSF LARMITN..E SDIYSLPPDP KYFDGYDNQS VVIMDDIMQN |
| hrv-18 | GTGKSMTTNF LARMITS..D SDIYSLPPDP KYFDGYDQQS VVIMDDIMQN |
| hrv-19 | GTGKSITTSF LARMITN..E SDVYSLPPDP KYFDGYDQQS VVIMDDIMQN |
| hrv-20 | GTGKSITTSI LARMLTT..E SDIYSLPPSP KYFDGYDQQS VVIMDDIMQN |
| hrv-21 | GTGKSMTTNF LARMITN..D SDIYSLPPDP KYFDGYDQQS VVIMDDIMQN |
| hrv-22 | GTGKSITTSF LARMLTN..E SDIYSLPPDP KYFDGYDQQS VVIMDDIMQN |
| dq473497-23 | GAGKSITTNF LAKMITN..D SDIYSLPPDP KYFDGYDQQS VVIMDDIMQN |
| hrv-24 | GTGKSMTTNF LARMITN..D SDIYSLPPDP KYFDGYDQQS VVIMDDIMQN |
| ef173416-24 | GTGKSMTTNF LARMITN..D SDIYSLPPDP KYFDGYDQQS VVIMDDIMQN |
| hrv-25 | GTGKSMTTNF LARMITN..D SDIYSLPPDP KYFDGYDQQS VVIMDDIMQN |
| dq473508-28 | GTGKSITTSI LARMLTT..E SDIYSLPPSP KYFDGYDQQS VVIMDDIMQN |
| hrv-29 | GTGKSMTTSF LARMITN..E SDIYSLPPDP KYFDGYDQQS VVIMDDVMQN |
| dq473512-30 | GAGKSITTNF LAKMITN..D SDIYSLPPDP KYFDGYDQQS VVIMDDIMQN |
| hrv-30 | GAGKSITTNF LAKMITN..D SDIYSLPPDP KYFDGYDQQS VVIMDDIMQN |
| hrv-31 | GTGKSMTTNF LARMITN..D SDIYSLPPDP KYFDGYDQQS VVIMDDIMQN |
| hrv-32 | GTGKSITTSF LARMITN..E SDIYSLPPDP KYFDGYDQQS VVIMDDIMQN |
| hrv-33 | GAGKSMTTNF LARMITN..D SDIYSLPPDP KYFDGYDQQS VVIMDDIMQN |
| hrv-34 | GTGKSMTTNF LARMITN..D SDIYSLPPDP KYFDGYDQQS VVIMDDIMQN |
| dq473501-34 | GTGKSMTTNF LARMITN..D SDIYSLPPDP KYFDGYDQQS VVIMDDIMQN |
| dq473505-36 | GTGKSLATSV LARMLTV..E TDIYSLPPDP KYFDGYDQQS VVIMDDIMQN |
| hrv-38 | GTGKSITTSF LARMITN..E SDVYSLPPDP KYFDGYDQQS VVIMDDIMQN |
| dq473495-38 | GTGKSITTSF LARMITN..E SDVYSLPPDP KYFDGYDQQS VVIMDDIMQN |
| ay751783-39 | GTGKSITTSF LARMITN..D SDIYSLPPDP KYFDGYDQQS VVIMDDIMQN |
| hrv-40 | GTGKSMTTNF LARMITS..D SDIYSLPPDP KYFDGYDQQS VVIMDDVMQN |
| dq473491-41 | GTGKSITTSF LARMITN..E SDIYSLPPDP KYFDGYDQQS VVIMDDIMQN |
| hrv-43 | GTGKSITTSF LARMITN..E SDVYSLPPDP KYFDGYDQQS VVIMDDIMQN |
| dq473499-44 | GTGKSMTTSF LARMITN..E SDIYSLPPDP KYFDGYDQQS VVIMDDVMQN |
| hrv-45 | GSGKSVCTNI LARMITN..P SDIYSLPPNP KYFDGYHQQT VVIMDDVMQN |
| dq473506-46 | GTGKSITTSI LARMLTS..E SDIYSLPPSP KYFDGYDQQS VVIMDDIMQN |
| hrv-47 | GTGKSMTTNF LARMITN..D SDIYSLPPDP KYFDGYDQQS VVIMDDIMQN |
| dq473496-49 | GAGKSITTNF LARMITN..D SDIYSLPPDP KYFDGYDQQS VVIMDDIMQN |
| hrv-49-f04 | GAGKSITTNF LARMITN..D SDIYSLPPDP KYFDGYDQQS VVIMDDIMQN |
| hrv-50 | GTGKSMTTSF LARMITN..E SDIYSLPPDP KYFDGYDQQS VVIMDDVMQN |
| hrv-51 | GTGKSIATSI LARMLTT..E SDIYSLPPSP KYFDGYDQQS VVIMDDIMQN |
| dq473507-53 | GTGKSITTSI LARMLTS..E SDIYSLPPSP KYFDGYDQQS VVIMDDIMQN |
| hrv-54 | GTGKSMTTNF LARMITN..D SDIYSLPPDP KYFDGYDQQS VVIMDDVMQN |
| hrv-54-f05 | GTGKSMTTNF LARMITN..D SDIYSLPPDP KYFDGYDQQS VVIMDDVMQN |
| dq473511-55 | GTGKSMTTNF LARMITN..D SDIYSLPPDP KYFDGYDQQS VVIMDDIMQN |
| hrv-56 | GTGKSMTTNF LARMITT..E SDIYSLPPDP KYFDGYDQQS VVIMDDVMQN |
| hrv-57 | GTGKSMTTNF LARMITN..D SDIYSLPPDP KYFDGYDQQS VVIMDDIMQN |
| hrv-58 | GTGKSLATSV LARMLTV..E TDIYSLPPDP KYFDGYDQQS VVIMDDIMQN |
| dq473500-59 | GTGKSMTTNF LARMITN..D SDIYSLPPDP KYFDGYDQQS VVIMDDIMQN |
| hrv-60 | GTGKSITTSF LARMITN..E SDIYSLPPDP KYFDGYDQQS VVIMDDIMQN |
| hrv-61 | GTGKSITTNF LARMITN..E SDIYSLPPDP KYFDGYDQQS VVIMDDIMQN |
| hrv-62 | GTGKSMTTNF LARMITN..D SDIYSLPPDP KYFDGYDQQS VVIMDDIMQN |
| hrv-63 | GTGKSMTTNF LARMITN..D SDIYSLPPDP KYFDGYDQQS VVIMDDIMQN |
| hrv-64 | GTGKSITTSF LARMLTN..E SDIYSLPPDP KYFDGYDQQS VVIMDDIMQN |
| ef173417-64 | GTGKSITTSF LARMLTN..E SDIYSLPPDP KYFDGYDQQS VVIMDDIMQN |
| hrv-65 | GTGKSIATSI LARMLTT..E SDIYSLPPSP KYFDGYDQQS VVIMDDIMQN |
| hrv-66 | GTGKSMTTNF LARMITS..D SDIYSLPPDP KYFDGYDQQS VVIMDDIMQN |
| hrv-67 | GTGKSITTSF LARMITN..E SDIYSLPPDP KYFDGYDQQS VVIMDDIMQN |
| hrv-68 | GTGKSITTSI LARMLTT..E SDIYSLPPSP KYFDGYDQQS VVIMDDIMQN |
| hrv-71 | GTGKSIATSI LARMITT..E SDIYSLPPSP KYFDGYDQQS VVIMDDIMQN |
| dq473492-73 | GTGKSITTSF LARMITN..E SDIYSLPPDP KYFDGYDQQS VVIMDDIMQN |
| dq473494-74 | GTGKSITTSF LARMITN..E SDIYSLPPDP KYFDGYDQQS VVIMDDIMQN |
| dq473510-75 | GTGKSITTSF LARMITN..E SDVYSLPPDP KYFDGYDQQS VVIMDDIMQN |
| dq473502-76 | GAGKSMTTNF LARMITN..D SDIYSLPPDP KYFDGYDQQS VVIMDDIMQN |
| hrv-76 | GAGKSMTTNF LARMITN..D SDIYSLPPDP KYFDGYDQQS VVIMDDIMQN |
| hrv-77 | GTGKSMTTNF LARMITN..D SDIYSLPPDP KYFDGYDQQS VVIMDDIMQN |
| hrv-78 | GTGKSLATNF LGRMITS..E TDIYSLPPDP KHFDGYDQQT VVIMDDVMQN |
| ef173418-78 | GTGKSLATNF LGRMITS..E TDIYSLPPDP KHFDGYDQQT VVIMDDVMQN |
| hrv-80 | GTGKSIATSI LGRMLTN..E SDIYSLPPSP KYFDGYDQQS VVIMDDIMQN |
| hrv-81 | GTGKSITTSF LARMITN..E SDIYSLPPDP KYFDGYDNQS VVIMDDIMQN |
| hrv-81-f06 | GTGKSITTSF LARMITN..E SDIYSLPPDP KYFDGYDNQS VVIMDDIMQN |
| hrv-81-f07 | GTGKSITTSF LARMITN..E SDIYSLPPDP KYFDGYDNQS VVIMDDIMQN |
| hrv-82 | GTGKSITTSF LARMITN..E SDIYSLPPDP KYFDGYDQQS VVIMDDIMQN |
| dq473509-82 | GTGKSITTSF LARMITN..E SDIYSLPPDP KYFDGYDQQS VVIMDDIMQN |
| hrv-85 | GTGKSMTTNF LARMITS..D SDIYSLPPDP KYFDGYDQQS VVIMDDVMQN |
| pico-85 | GTGKSMTTNF LARMITS..D SDIYSLPPDP KYFDGYDQQS VVIMDDVMQN |
| dq473504-88 | GTGKSLATSV LARMLTV..E TDIYSLPPDP KYFDGYDQQS VVIMDDIMQN |
| a10937-89 | GTGKSLATSV LARMLTV..E TDIYSLPPDP KYFDGYDQQS VVIMDDIMQN |
| hrv-89 | GTGKSLATSV LARMLTV..E TDIYSLPPDP KYFDGYDQQS VVIMDDIMQN |
| hrv-89-f08 | GTGKSLATSV LARMLTV..E TDIYSLPPDP KYFDGYDQQS VVIMDDIMQN |
| hrv-89-f09 | GTGKSLATSV LARMLTV..E TDIYSLPPDP KYFDGYDQQS VVIMDDIMQN |
| m16248-89 | GTGKSLATSV LARMLTV..E TDIYSLPPDP KYFDGYDQQS VVIMDDIMQN |
| hrv-90 | GTGKSMTTNF LARMITN..D SDIYSLPPDP KYFDGYDQQS VVIMDDIMQN |
| hrv-94 | GTGKSITTSF LARMLTN..E SDIYSLPPDP KYFDGYDQQS VVIMDDIMQN |
| ef173419-94 | GTGKSITTSF LARMLTN..E SDIYSLPPDP KYFDGYDQQS VVIMDDIMQN |
| hrv-95 | GSGKSICTNI LARMITN..P SDIYSLPPDP KYFDGYHQQT VVIMDDVMQN |
| hrv-96 | GTGKSITTSF LARMITN..E SDIYSLPPDP KYFDGYDQQS VVIMDDIMQN |
| hrv-98 | GTGKSMTTSF LARMITN..D SDIYSLPPDP KYFDGYDQQS VVIMDDVMQN |
| hrv-100 | GTGKSMTTNF LARMITS..D SDIYSLPPDP KYFDGYDQQS VVIMDDIMQN |
| ef186077-qpm | GCGKSLASKV IARGLTS..E SNVYSLPPDP KYFDGYNQQE VVIMDDVGQN |
| ef077279-nat001 | GCGKSLASKI IARGLTN..E SNVYSLPPDP KYFDGYNQQE VVIMDDVGQN |
| ny1078 | GCGKSLASKI VARGLTN..E SNVYSLPPDP KYFDGYNQQE VVIMDDVGQN |
| ef582385-c024 | GCGKSLVTSI IARGLTK..P GDIYSLPPDP KHFDGYDQQE VVIMDDLGQN |
| ef077280-nat045 | GCGKSLITTI IARGLAT..E GNIYSLPPNP KHFDGYNQQD VVIMDDVCQN |
| ef582387-c026 | GCGKSLASKV IARGLTN..E SNVYSLPPDP KYFDGYNQQE VVIMDDVGQN |
| ef582386-c025 | GCGKSLATTV IARGLAD..S GNVYSLPPNP KHFDGYCQQQ VVMMDDLGQN |
| dq473485-03 | GSGKSLTTSI VGRALAEHFN SSVYSLPPDP KHFDGYQQQE VVIMDDLNQN |
| ef173422-03 | GSGKSLTTSI VGRALAEHFN SSVYSLPPDP KHFDGYQQQE VVIMDDLNQN |
| dq473490-04 | GSGKSLTTSL VGRAIADNYN SSVYSLPPDP KHFDGYQQQA VVILDDLNQN |
| hrv-05 | GSGKSLTTSL IGRAIADNCG SSVYSLPPDP KHFDGYQQQA VVILDDLNQN |
| dq473486-06 | GSGKSLTTSI VGRALAEHFN SSVYSLPPDP KHFDGYQQQE VVIMDDLNQN |
| 105355-14 | GSGKSLTTSI VGRAIAEHFN SAVYSLPPDP KHFDGYQQQE VVIMDDLNQN |
| k02121-14 | GSGKSLTTSI VGRAIAEHFN SAVYSLPPDP KHFDGYQQQE VVIMDDLNQN |
| ef173420-17 | GSGKSLTTSI IGRAIAEKFK STVYSLPPDP KHFDGYQQQE VVIMDDLNQN |
| hrv-26 | GSGKSLTTSL IGRAIADNYG SSVYSLPPDP KHFDGYQQQA VVILDDLNQN |
| hrv-27 | GSGKSLTTSL IGRSIADIHR SSVYSLPPDP KHFDGYQQQA VVILDDLNQN |
| ef173421-27 | GSGKSLTTSL IGRSIADIHR SSVYSLPPDP KHFDGYQQQA VVILDDLNQN |
| dq473487-35 | GSGKSLTTSI VGRALAEHFN SAVYSLPPDP KHFDGYQQQE VVIMDDLNQN |
| hrv-35 | GSGKSLTTSI VGRALAEHFN SAVYSLPPDP KHFDGYQQQE VVIMDDLNQN |
| ef173423-37 | GSGKSLTTSI VGRALAEHFN SAVYSLPPDP KHFDGYQQQE VVIMDDLNQN |
| hrv-42 | GSGKSLTTSL IGRAIADSCG SSVYSLPPDP KHFDGYQQQA VVILDDLNQN |
| dq473488-48 | GSGKSLTTSI IGRAIAERYK STVYSLPPDP KHFDGYQQQE VVIMDDLNQN |
| hrv-52 | GSGKSLTTSI IGRAIAERFK STVYSLPPDP KHFDGYQQQE VVIMDDLNQN |
| hrv-52-f10 | GSGKSLTTSI IGRAIAERFK STVYSLPPDP KHFDGYQQQE VVIMDDLNQN |
| ef173424-52 | GSGKSLTTSI IGRAIAERFK STVYSLPPDP KHFDGYQQQE VVIMDDLNQN |
| hrv-69 | GSGKSLTTSI IGRAIAERFK STVYSLPPDP KHFDGYQQQE VVIMDDLNQN |
| dq473489-70 | GSGKSLTTSI VGRAIAERFK STVYSLPPDP KHFDGYQQQE VVIMDDLNQN |
| hrv-72 | GSGKSLTTSI IGRAIAEQFN SAVYSLPPDP KHFDGYQQQE VVIMDDLNQN |
| hrv-79 | GSGKSLTTSI VGRALAEHFN SSVYSLPPDP KHFDGYQQQE VVIMDDLNQN |
| hrv-83 | GSGKSLTTSI VGRALAEHFN SAVYSLPPDP KHFDGYQQQE VVIMDDLNQN |
| hrv-84 | GSGKSLATSL IGRSIADNHK SSVYSLPPDP KHFDGYQQQA VVILDDLNQN |
| hrv-86 | GSGKSLTTSI VGRALAEHYN SAVYSLPPDP KHFDGYQQQE VVIMDDLNQN |
| hrv-91 | GSGKSLTTSI IGRAIAERFK STVYSLPPDP KHFDGYQQQE VVIMDDLNQN |
| hrv-92 | GSGKSLTTSI VGRALAEHLN SAVYSLPPDP KHFDGYQQQE VVIMDDLNQN |
| ef173425-93 | GSGKSLATSL IGRSIADIHK SSVYSLPPDP KHFDGYQQQA VVILDDLNQN |
| hrv-97 | GSGKSLTTSL IGRSIAEIYK SSVYSLPPDP KHFDGYQQQA VVILDDLNQN |
| hrv-99 | GSGKSLTTSL IGRAIADSCG SSVYSLPPDP KHFDGYQQQA VVILDDLNQN |
| | 1351 1400 |
| annotation | .......... .......... .......... .......... .......... |
| af499637-cva13 | PDGMDMKLFC QMVSTVEFIP PMASLEEKGI LFTSDYVLAS TNSSTITPPT |
| af546702-cva21 | PDGMDMKLFC QMVSTVEFIP PMASLEEKGI LFTSDYVLAS TNSHSIVPPT |
| v01149-pv1m | PDGADMKLFC QMVSTVEFIP PMASLEEKGI LFTSNYVLAS TNSSRISPPT |
| d00239-01 | PDGEDMTLFC QMVSSVTFIP PMADLPDKGK PFDSRFVLCS TNHSLLAPPT |
| pico-01 | PDGEDMTLFC QMVSSVTFIP PMADLPDKGK PFDSRFILCS TNHSLLAPPT |
| hrv-01 | PDGEDMTLFC QMVSSVTFIP PMADLPDKGK PFDSRFILCS TNHSLLAPPT |
| x02316-02 | PAGDDMTLFC QMVSSVTFIP PMADLPDKGK AFDSRFVLCS TNHSLLTPPT |
| hrv-07 | PNGEDMTLFC QMVSSVPFIP PMADLPDKGK PFTSKFVLAS TNHTLLTPPT |
| dq473503-07 | PNGEDMTLFC QMVSSVPFIP PMADLPDKGK PFTSKFVLAS TNHTLLTPPT |
| hrv-08 | PNGEDMSTFC QMVSSVNYVV PMADLPDKGT LFSSDYVFCS TNQHILTPPT |
| pico-09 | PGGEDMSLFC QMVSSVTFIP PMADLPDKGK PFDSRFVFCS TNHSLLAPPT |
| hrv-09 | PGGEDMSLFC QMVSSVTFIP PMADLPDKGK PFDSRFVFCS TNHSLLAPPT |
| hrv-09-f01 | PGGEDMSLFC QMVSSVTFIP PMADLPDKGK PFDSRFVFCS TNHSLLAPPT |
| hrv-09-f02 | PGGEDMSLFC QMVSSVTFIP PMADLPDKGK PFDSRFVFCS TNHSLLAPPT |
| dq473498-10 | PSGEDMTLFC QMVSSVTFIP PMADLPDKGK PFDSRFVLCS TNHNLLAPPT |
| hrv-10 | PSGEDMTLFC QMVSSVTFIP PMADLPDKGK PFDSRFVLCS TNHNLLAPPT |
| ef173414-11 | PSGDDMTLFC QMVSSVTFIP PMADLPDKGK PFDSRFVLCS TNHSMLAPPT |
| ef173415-12 | PSGEDMTLFC QMVSSVPFIP PMADLPDKGK HFTSEYVLAS TNHSILNPPT |
| hrv-13 | PDGEDMSLFC QMVSSVTFIP PMADLPDKGK PFDSRFVLCS TNHSLLAPPT |
| hrv-13-f03 | PDGEDMSLFC QMVSSVTFIP PMADLPDKGK PFDSRFVLCS TNHSLLAPPT |
| dq473493-15 | PGGEDMSLFC QMVSSVTFIP PMADLPDKGK PFDSRFVFCS TNHSLLAPPT |
| 124917-16 | PGGEDMTLFC QMVSSVTFIP PMADLPDKGK PFDSRFVLCS TNHSLLAPPT |
| hrv-18 | PSGDDMTLFC QMVSSVTFIP PMADLPDKGK PFDSRFVLCS TNHNLLAPPT |
| hrv-19 | PDGEDMSLFC QMVSSVTFIP PMADLPDKGK PFDSRFVFCS TNHSLLAPPT |
| hrv-20 | PSGEDMSLFC QMVSSVPFIP PMADLPDKGK PFSSDYVLAS TNHTLLHPPT |
| hrv-21 | PSGDDMTLFC QMVSSVTFIP PMADLPDKGK PFDSRFVLCS TNHSLLTPPT |
| hrv-22 | PDGEDMSLFC QMVSSVTFIP PMADLPDKGK PFDSRFVFCS TNHSLLAPPT |
| dq473497-23 | PTGDDMTLFC QMVSSVTFIP PMADLPDKGK AFDSRFVLCS TNHSLLAPPT |
| hrv-24 | PSGEDMTLFC QMVSSVTFIP PMADLPDKGK PFDSRFVLCS TNHGMLAPPT |
| ef173416-24 | PSGEDMTLFC QMVSSVTFIP PMADLPDKGK PFDSRFVLCS TNHGMLAPPT |
| hrv-25 | PSGEDMTLFC QMVSSVTFIP PMADLPDKGK PFDSRFVLCS TNHNMLAPPT |
| dq473508-28 | PSGEDMSLFC QMVSSVPFIP PMADLPDKGK PFSSDYVLAS TNHTLLHPPT |
| hrv-29 | PSGDDMTLFC QMVSSVTFIP PMADLPDKGK PFDSRFVLCS TNHNMLAPPT |
| dq473512-30 | PAGDDMTLFC QMVSSVTFIP PMADLPDKGK TFDSRFVLCS TNHSLLAPPT |
| hrv-30 | PAGDDMTLFC QMVSSVTFIP PMADLPDKGK AFDSRFVLCS TNHSLLAPPT |
| hrv-31 | PSGEDMTLFC QMVSSVTFIP PMADLPDKGK PFDSRFVLCS TNHNLLAPPT |
| hrv-32 | PGGEDMSLFC QMVSSVTFIP PMADLPDKGK PFDSRFVFCS TNHSLLAPPT |
| hrv-33 | PSGDDMTLFC QMVSSVTFIP PMADLPDKGK PFDSRFVLCS TNHSMLAPPT |
| hrv-34 | PSGDDMTLFC QMVSSVTFIP PMADLPDKGK PFDSRFVLCS TNHNLLAPPT |
| dq473501-34 | PSGDDMTLFC QMVSSVTFIP PMADLPDKGK PFDSRFVLCS TNHNLLAPPT |
| dq473505-36 | PSGEDMTLFC QMVSSVPFIP PMADLPDKGK PFTSKFVLAS TNHTMLTPPT |
| hrv-38 | PDGEDMSLFC QMVSSVTFIP PMADLPDKGK PFDSRFVFCS TNQSLLAPPT |
| dq473495-38 | PDGEDMSLFC QMVSSVTFIP PMADLPDKGK PFDSRFVFCS TNQSLLAPPT |
| ay751783-39 | PTGEDMTLFC QMVSSVTFIP PMADLPDKGK AFDSRFVLCS TNHSLLAPPT |
| hrv-40 | PSGDDMTLFC QMVSSVTFIP PMADLPDKGK PFDSRFVLCS TNHNLLAPPT |
| dq473491-41 | PDGEDMSLFC QMVSSVTFIP PMADLPDKGK PFDSRFVLCS TNHSLLAPPT |
| hrv-43 | PDGEDMSLFC QMVSSVTFIP PMADLPDKGK PFDSRFVFCS TNQTLLAPPT |
| dq473499-44 | PSGDDMTLFC QMVSSVTFIP PMADLPDKGK PFDSRFVLCS TNHNMLAPPT |
| hrv-45 | PDGEDMSTFC QMVSSVNYVV PMADLPDKGT LFSSDYVFCS TNQHVLIPPT |
| dq473506-46 | PSGEDMSLFC QMVSSVPFIP PMADLPDKGK PFSSDYVLAS TNHTLLHPPT |
| hrv-47 | PSGEDMTLFC QMVSSVTFIP PMADLPDKGK PFDSRFVLCS TNHNLLAPPT |
| dq473496-49 | PAGDDMTLFC QMVSSVTFIP PMADLPDKGK AFDSRFVLCS TNHSLLAPPT |
| hrv-49-f04 | PAGDDMTLFC QMVSSVTFIP PMADLPDKGK AFDSRFVLCS TNHSLLAPPT |
| hrv-50 | PSGDDMTLFC QMVSSVTFIP PMADLPDKGK PFDSRFVLCS TNHSLLAPPT |
| hrv-51 | PNGEDMSLFC QMVSSVPFIP PMADLPDKGK PFSSDYVLAS TNHNLLHPPT |
| dq473507-53 | PSGEDMSLFC QMVSSVPFIP PMADLPDKGK PFSSDYVLAS TNHTLLHPPT |
| hrv-54 | PSGDDMTLFC QMVSSVTFIP PMADLPDKGK PFDSRFVLCS TNHNLLAPPT |
| hrv-54-f05 | PSGDDMTLFC QMVSSVTFIP PMADLPDKGK PFDSRFVLCS TNHNLLAPPT |
| dq473511-55 | PSGDDMTLFC QMVSSVTFIP PMADLPDKGK PFDSRFVLCS TNHSMLAPPT |
| hrv-56 | PSGEDMTLFC QMVSSVTFIP PMADLPDKGK PFDSRFVLCS TNHNLLAPPT |
| hrv-57 | PSGEDMTLFC QMVSSVTFIP PMADLPDKGK PFDSRFVLCS TNHSMLAPPT |
| hrv-58 | PNGEDMTLFC QMVSSVPFIP PMADLPDKGK PFTSKFVLAS TNHTLLTPPT |
| dq473500-59 | PSGDDMTLFC QMVSSVTFIP PMADLPDKGK PFDSRFVLCS TNHNMLAPPT |
| hrv-60 | PDGEDMSLFC QMVSSVAFIP PMADLPDKGK PFDSRFVFCS TNQSLLAPPT |
| hrv-61 | PDGEDMALFC QMVSSVTFIP PMADLPDKGK PFDSRFVLCS TNHSMLAPPT |
| hrv-62 | PSGEDMTLFC QMVSSVTFIP PMADLPDKGK PFDSRFVLCS TNHNMLAPPT |
| hrv-63 | PSGEDMTLFC QMVSSVTFIP PMADLPDKGK PFDSRFVLCS TNHNMLAPPT |
| hrv-64 | PDGEDMSLFC QMVSSVTFIP PMADLPDKGK PFDSRFVFCS TNHSLLAPPT |
| ef173417-64 | PDGEDMSLFC QMVSSVTFIP PMADLPDKGK PFDSRFVFCS TNHSLLAPPT |
| hrv-65 | PNGEDMSLFC QMVSSVPFIP PMADLPDKGK PFSSDYVLAS TNHNLLHPPT |
| hrv-66 | PSGDDMTLFC QMVSSVTFIP PMADLPDKGK PFDSKFVLCS TNHHLLAPPT |
| hrv-67 | PGGEDMSLFC QMVSSVTFIP PMADLPDKGK PFDSRFVFCS TNHSLLAPPT |
| hrv-68 | PSGEDMSLFC QMVSSVPFIP PMADLPDKGK PFSSDYVLAS TNHTLLHPPT |
| hrv-71 | PNGEDMSLFC QMVSSVPFIP PMADLPDKGK PFSSDYVLAS TNHSLLHPPT |
| dq473492-73 | PDGEDMSLFC QMVSSVTFIP PMADLPDKGK PFDSRFVLCS TNHSLLAPPT |
| dq473494-74 | PGGEDMSLFC QMVSSVTFIP PMADLPDKGK PFDSRFVFCS TNHSLLAPPT |
| dq473510-75 | PDGEDMSLFC QMVSSVTFIP PMADLPDKGK PFDSRFVFCS TNQTLLAPPT |
| dq473502-76 | PSGEDMTLFC QMVSSVTFIP PMADLPDKGK PFDSRFVLCS TNHSMLAPPT |
| hrv-76 | PSGEDMTLFC QMVSSVTFIP PMADLPDKGK PFDSRFVLCS TNHSMLAPPT |
| hrv-77 | PSGDDMTLFC QMVSSVTFIP PMADLPDKGK PFDSRFVLCS TNHHLLAPPT |
| hrv-78 | PDGEDMSLFC QMVSSVPFIP PMADLPDKGK PFSSRYVLCS TNHTILTPPT |
| ef173418-78 | PDGEDMSLFC QMVSSVPFIP PMADLPDKGK PFSSRYVLCS TNHTILTPPT |
| hrv-80 | PSGEDMSLFC QMVSSVPFIP PMADLPDKGK PFSSDFVLAS TNHTLLHPPT |
| hrv-81 | PGGEDMTLFC QMVSSVTFIP PMADLPDKGK PFDSRFVLCS TNHSLLAPPT |
| hrv-81-f06 | PGGEDMTLFC QMVSSVTFIP PMADLPDKGK PFDSRFVLCS TNHSLLAPPT |
| hrv-81-f07 | PGGEDMTLFC QMVSSVTFIP PMADLPDKGK PFDSRFVLCS TNHSLLAPPT |
| hrv-82 | PDGEDMSLFC QMVSSVTFIP PMADLPDKGK PFDSRFVFCS TNQSLLAPPT |
| dq473509-82 | PDGEDMSLFC QMVSSVTFIP PMADLPDKGK PFDSRFVFCS TNQSLLAPPT |
| hrv-85 | PSGDDMTLFC QMVSSVTFIP PMADLPDKGK PFDSRFVLCS TNHNLLAPPT |
| pico-85 | PSGDDMTLFC QMVSSVTFIP PMADLPDKGK PFDSRFVLCS TNHNLLAPPT |
| dq473504-88 | PNGEDMTLFC QMVSSVPFIP PMADLPDKGK PFTSKFVLAS TNHTLLTPPT |
| a10937-89 | PSGEDMTLFC QMVSSVPFIP PMADLPDKGK PFTSKFVLAS TNHTLLTPPT |
| hrv-89 | PSGEDMTLFC QMVSSVPFIP PMADLPDKGK PFTSKFVLAS TNHTLLTPPT |
| hrv-89-f08 | PSGEDMTLFC QMVSSVPFIP PMADLPDKGK PFTSKFVLAS TNHTLLTPPT |
| hrv-89-f09 | PSGEDMTLFC QMVSSVPFIP PMADLPDKGK PFTSKFVLAS TNHTLLTPPT |
| m16248-89 | PSGEDMTLFC QMVSSVPFIP PMADLPDKGK PFTSKFVLAS TNHTLLTPPT |
| hrv-90 | PSGEDMTLFC QMVSSVTFIP PMADLPDKGK SFDSRFVLCS TNHGMLAPPT |
| hrv-94 | PDGEDMSLFC QMVSSVTFIP PMADLPDKGK PFDSRFVFCS TNHSLLTPPT |
| ef173419-94 | PDGEDMSLFC QMVSSVTFIP PMADLPDKGK PFDSRFVFCS TNHSLLTPPT |
| hrv-95 | PNGEDMSTFC QMVSSVNYVV PMADLPDKGT LFSSDYVFCS TNQHILTPPT |
| hrv-96 | PDGEDMSLFC QMVSSVTFIP PMADLPDKGK PFDSRFVLCS TNHSLLAPPT |
| hrv-98 | PSGDDMTLFC QMVSSVTFIP PMADLPDKGK PFDSRFVLCS TNHNLLAPPT |
| hrv-100 | PSGDDMTLFC QMVSSVTFIP PMADLPDKGK PFDSRFVLCS TNHNLLAPPT |
| ef186077-qpm | PDGKDLSLFC QMVSSTEFIT PMANLEDKGR AFTSDYVIAS TNMSVLTPPT |
| ef077279-nat001 | PDGRDLSLFC QMVSSTEFIT PMANLEDKGR AFTSDYVIAS TNMSILTPPT |
| ny1078 | PDGKDLSFFC QMVSSTEFIT PMANLEDKGR AFTSDYVIAS TNMTVLTPPT |
| ef582385-c024 | PDGKDLSMFC QMVSTTNFIP PMAALEDKGK TFTSKYILAS TNLLNLQPPT |
| ef077280-nat045 | PDGEDMNMFC QMVSTADFHV PMAAIEDKGK SFTSKYVLAS TNMAALQPPT |
| ef582387-c026 | PDGKDLSLFC QMVSSTEFIT PMANLEDKGR AFTSDYVIAS TNMTVLTPPT |
| ef582386-c025 | PDGQDLAMFC QMVSTTDFIV PMAALEDKGK SFTSDYVLAS TNLNQLSPPT |
| dq473485-03 | PDGQDISMFC QMVSSVDFLP PMASLDNKGM LFTSNFVLAS TNSNTLSPPT |
| ef173422-03 | PDGQDISMFC QMVSSVDFLP PMASLDNKGM LFTSNFVLAS TNSNTLSPPT |
| dq473490-04 | PNGEDISTFC QMVSSVDFIP PMANLDNKGM LFTSSFVLAS TNSQTLNPPT |
| hrv-05 | PNGEDISTFC QMVSSVDFIP PMANLDNKGM LFTSNFVLAS TNSQTLNPPT |
| dq473486-06 | PDGQDISMFC QMVSTVDFIP PMANLDNKGM LFTSNFVLAS TNSNTLSPPT |
| 105355-14 | PDGQDISMFC QMVSSVDFLP PMASLDNKGM LFTSNFVLAS TNSNTLSPPT |
| k02121-14 | PDGQDISMFC QMVSSVDFLP PMASLDNKGM LFTSNFVLAS TNSNTLSPPT |
| ef173420-17 | PDGQDISMFC QMVSSVDFIP PMASLDNKGI SFTSNFVLAS TNSNTLSPPT |
| hrv-26 | PNGEDISTFC QMVSSVDFIP PMANLDNKGM LFTSNFVLAS TNSQTLNPPT |
| hrv-27 | PNGEDISTFC QMVSSVDFIP PMANLDNKGM LFTSSFVLAS TNSQTLNPPT |
| ef173421-27 | PNGEDISTFC QMVSSVDFIP PMANLDNKGM LFTSSFVLAS TNSQTLNPPT |
| dq473487-35 | PDGQDISMFC QMVSSVDFLP PMASLDNKGM LFTSNFVLAS TNSNTLSPPT |
| hrv-35 | PDGQDISMFC QMVSSVDFLP PMASLDNKGM LFTSNFVLAS TNSNTLSPPT |
| ef173423-37 | PDGQDISMFC QMVSSVDFIP PMANLDNKGM LFTSNFVLAS TNSNTLSPPT |
| hrv-42 | PNGEDISTFC QMVSSVDFIP PMANLDNKGM LFTSNFVLAS TNSQTLNPPT |
| dq473488-48 | PDGQDISMFC QMVSSVDFIP PMASLDNKGI LFTSNFVLAS TNSNTLNPPT |
| hrv-52 | PDGQDISMFC QMVSSVDFIP PMASLDNKGI LFTSNFVLAS TNSNTLSPPT |
| hrv-52-f10 | PDGQDISMFC QMVSSVDFIP PMASLDNKGI LFTSNFVLAS TNSNTLSPPT |
| ef173424-52 | PDGQDISMFC QMVSSVDFIP PMASLDNKGI LFTSNFVLAS TNSNTLSPPT |
| hrv-69 | PDGQDISMFC QMVSSVDFIP PMASLDNKGI LFTSNFVLAS TNSNTLSPPT |
| dq473489-70 | PDGQDVSMFC QMVSSVDFIP PMASLDNKGI LFTSNFVLAS TNSNTLSPPT |
| hrv-72 | PDGQDISMFC QMVSSVDFLP PMASLDNKGM LFTSNFVLAS TNSNTLSPPT |
| hrv-79 | PDGQDISMFC QMVSSVDFLP PMASLDNKGM LFTSNFVLAS TNSNTLSPPT |
| hrv-83 | PDGQDISMFC QMVSSVDFLP PMASLDNKGM LFTSNFVLAS TNSNTLSPPT |
| hrv-84 | PNGEDISTFC QMVSXVDFVP PMANLDNKGM LFTSSFVLAS TNSQTLNPPT |
| hrv-86 | PDGQDISMFC QMVSSVDFLP PMASLDNKGM LFTSNFVLAS TNSNTLSPPT |
| hrv-91 | PDGQDISMFC QMVSXVDFIP PMASLDNKGI LFTSNFVLAS TNSNTLSPPT |
| hrv-92 | PDGQDISMFC QMVSSVDFIP PMASLDNKGM LFTSNFVLAS TNSNTLSPPT |
| ef173425-93 | PNGEDISTFC QMVSSVDFIP PMANLDNKGM LFTSSFVLAS TNSQTLNPPT |
| hrv-97 | PNGEDVSTFC QMVSSVDFIP PMANLDNKGM LFTSSFVLAS TNSQTLNPPT |
| hrv-99 | PNGEDISTFC QMVSSVDFIP PMANLDNKGM LFTSNYVLAS TNSQTLNPPT |
| | 1401 1450 |
| annotation | .......... .......... .......... .......... .......... |
| af499637-cva13 | VAHSDALSRR FAFDMEICPL TQY.TIKGKL NMAEATRLCK DCPQPANFKK |
| af546702-cva21 | VAHSDALTRR FAFDVEVYTM SEH.SVKGKL NMATATQLCK DCPTPANFKK |
| v01149-pv1m | VAHSDALARR FAFDMDIQVM NEY.SRDGKL NMAMATEMCK NCHQPANFKR |
| d00239-01 | ISSLPAMNRR FFFDLDIVVH DNYKDAQGKL NVSKAFQPCN VNTKIGN.AK |
| pico-01 | ISSLPAMNRR FFFDLDIVVH DNYKDTQGKL DVSKAFRPCN VNTKIGN.AK |
| hrv-01 | ISSLPAMNRR FFFDLDIVVH DNYKDTQGKL DVSKAFRPCN VNTKIGN.AK |
| x02316-02 | ITSLPAMNRR FFLDLDIIVH DNFKDPQGKL NVAAAFRPCD VDNRIGN.AR |
| hrv-07 | VSSLPAMSRR FYFDLDIQVK KDY.LVDGKL DIAKSFKPCD VNNKIGN.AK |
| dq473503-07 | VSSLPAMSRR FYFDLDIQVK KDY.LVDGKL DIAKSFKPCD VNNKIGN.AK |
| hrv-08 | ISTLPALNRR LFLDLTIKVN PKY.LESGKL NLDCALKACD QEQKIGN.AR |
| pico-09 | ITSLSAMNRR FYLDLDIVVN ENFRDSQGKL NVPKSFKPCD IDNKIGN.AK |
| hrv-09 | ITSLSAMNRR FYLDLDIVVN ENFRDSQGKL DVSKSFRPCD IDNKIGN.AK |
| hrv-09-f01 | ITSLSAMNRR FFLDLDIIVN ENFRDSQGKL DVPKSFKPCD IDNKIGN.AK |
| hrv-09-f02 | ITSLSAMNRR FFLDLDIIVN ENFRDSQGKL DVPKSFKPCD IDNKIGN.AK |
| dq473498-10 | ITSLPAMNRR FFFDLDIIVC EKYKDPQGKL NVTAAFRPCD VDAKIGN.AR |
| hrv-10 | ITSLPAMNRR FFFDLDIIVC EKYKDPQGKL NVTAAFRPCD VDAKIGN.AR |
| ef173414-11 | ITSLPAMNRR FFLDLDIVVN EKYKDLQGKL NVTAAFQPCD VNVRIGN.AR |
| ef173415-12 | ITSTTAMNRR FYLDLDIVVH PRFQINQGKL NLVAALKPCD .TGKVGN.AT |
| hrv-13 | ITSLQAMNRR FFLDLDIVVN DNYKDAQGKL DVSKAFRPCD VKTKIGN.AR |
| hrv-13-f03 | ITSLQAMNRR FFLDLDIVVH DNYKDAQGKL DVSKAFKPCD IDTKIGN.AR |
| dq473493-15 | ITSLSAMNRR FFLDLDIVIH DNYKDNQGKL DVSKSFKPCD VDNKIGN.AK |
| 124917-16 | ISSLPAMNRR FYLDLDILVH DNYKDNQGKL DVSRAFRLCD VDSKIGN.AK |
| hrv-18 | ITSLPAMNRR FFLDLDIIVC DKYKDSQGKL NVSAAFQLCD VNTKVGN.AR |
| hrv-19 | ITSLSAMNRR FYLDLDIIVH DNYKDGQGKL DVPKSFKPCD VDSKIGN.AK |
| hrv-20 | ISCTTAMNRR FFLDLDIIVK DDYKLGHGKL NLQCALKPCN .VGKIGN.AK |
| hrv-21 | ITSLPAMNRR FFMDLDIIVC DKYKDAQGKL NVSAAFKPCD VDTKIGN.AR |
| hrv-22 | ITSLSAMNRR FFLDLDIVVH XXYKDNLGKL DVPKSFRPCD VDNKIGN.AK |
| dq473497-23 | ITSLPAMNRR FFFDLDIIVH DNFKDSQGKL NVAAAFKPCD VDIKIGN.AR |
| hrv-24 | ITSLPAMNRR FFLDLDIVVN GKYKDSQGKL NVSAAFRPCD VDAKIGN.AR |
| ef173416-24 | ITSLPAMNRR FFLDLDIVVN GKYKDSQGKL NVSAAFRPCD VDAKIGN.AR |
| hrv-25 | ITSLPAMNRR FFFDLDIVVC DKYKDPQGKL NVSAAFRPCD IDTKIGN.AR |
| dq473508-28 | ITCTTAMNRR FFLDLDIVVK DEYKLSQGKL NLQCALKPCN .VGKIGN.AK |
| hrv-29 | ITSLPAMNRR FFFDLDIVVC DKYKDSQGKL NVSAAFQPCD VDIKIGN.AR |
| dq473512-30 | ITSLPAMNRR FFLDLDIIVH DRFKDPQGKL NVAAAFRPCD VDNKIGN.AR |
| hrv-30 | ITSLPAMNRR FFLDLDIIVH DRFKDPQGKL NVAAAFRPCD VDNKIGN.AR |
| hrv-31 | ITSLPAMNRR FFLDLDIXVC DKYKDPQGKL NVAAAFRPCD VDTKIGN.AR |
| hrv-32 | ITSLSAMNRR FYLDLDIVVN ENYKDTQGKL XVPKSFKPCD IDNKIGN.AK |
| hrv-33 | ITSLPAMNRR FYLDLDIVVS EKYKDQQGKL NVAAAFQPCD VNAKIGN.AR |
| hrv-34 | ITSLPAMNRR FFLDLDIVVC EKYKDSQGKL NVSAAFRPCD VNTKIGN.AR |
| dq473501-34 | ITSLPAMNRR FFLDLDIVVC EKYKDSQGKL NVSAAFRPCD VNTKIGN.AR |
| dq473505-36 | VSSLPAMSRR FYFDLDIQVK KDY.LLDGKL DIAKSFRPCD VNTKIGN.AK |
| hrv-38 | ITSLSAMNRR FFLDLDITVH DNYKDNQGKL NVPKSFKPCD VDNKIGN.AK |
| dq473495-38 | ITSLSAMNRR FFLDLDITVH DNYKDNQGKL NVPKSFKPCD VDNKIGN.AK |
| ay751783-39 | ITSLPAMNRR FFLDLDIIVH DNYKDAQGKL NVAAAFRPCD VNTKIGN.AR |
| hrv-40 | ITSLPAMNRR FFLDLDIIVC DKYKVAQGKL NVSAAFKPCD VNTKIGN.AK |
| dq473491-41 | ITSLQAMNRR FFLDLDIVVH ENYKDAQGKL DVSRAFKPCD VDTKIGN.AR |
| hrv-43 | ITSLPAMNRR FFLDLDIVVH DNYKDAQDKL NVPKAFKPCD VDNKIGN.AR |
| dq473499-44 | ITSLPAMNRR FFFDLDIVVC DKYKDSQGKL NVSAAFQPCD VDVKIGN.AR |
| hrv-45 | ISTIPALNRR LFFDLTVKVN PRY.QEAGKL NLDCALKPCN HELKVGN.AR |
| dq473506-46 | ITCTTAMNRR FFLDLDIIVK DEYKLNQGKL NLQCALRPCK .EGKIGN.AK |
| hrv-47 | ITSLPAMNRR FFLDLDIVVC DKYKDPQGKL NVAAAFQPCD VNTKIGN.AR |
| dq473496-49 | ITSLPAMNRR FFLDLDIIVH DNFKDSQGKL NVAAAFQPCN VDNKIGN.AR |
| hrv-49-f04 | ITSLPAMNRR FFLDLDILVH DNFKDSQGKL NVAAAFQPCD VDNKIGN.AR |
| hrv-50 | IXSLPAMNRR FFLDLDIIVC DKYKDSQGKL NVSAAFQPCD VNTKIGN.AR |
| hrv-51 | ITCTTAMNRR FFLDLDIVVK DVFKINQGRL NLQCALKPCK .EGKVGN.AK |
| dq473507-53 | ITCTTAMNRR FFLDLDIIVK DDYKLGQGKL NLQCALKPCK .EGKIGN.AK |
| hrv-54 | ITSLPAMNRR FFLDLDIVVC DKYKDSQGKL NVSAAFKPCD VNTKIGN.AK |
| hrv-54-f05 | ITSLPAMNRR FFLDLDIVVC DKYKDSQGKL NVSAAFKPCD VNTKIGN.AK |
| dq473511-55 | ITSLPAMNRR FFMDLDIIVN DKYKDSQGKL NVSAAFKPCD VDVKIGS.AR |
| hrv-56 | ITSLPAMNRR FFFDLDIIVC DKYKDPQGKL NVSAAFQPCD VDTKIGN.AR |
| hrv-57 | ITSLPAMNRR FFMDLDIIVN DKYKDAQGKL NVSAAFKPCD VDTKIGN.AR |
| hrv-58 | VSSLPAMSRR FYFDLDIQVK KDY.LLDGKL DIAKSFRLCD INTKIGN.AR |
| dq473500-59 | ITSLPAMNRR FYLDLDIIVN DRYKDPQGKL NVSAAFQPCD VDAKVGN.AR |
| hrv-60 | ITSLSAMNRR FYLDLDITVH DNYKDNLGKL DVPKAFKPCD VDTKIGN.AK |
| hrv-61 | ITSLQAMNRR FFLDLDIVVH DNYKDSQGKL NVSKAFKPCD VGTKIGN.AR |
| hrv-62 | ITSLPAMNRR FFFDLDIVVC DKYKDSQGKL NVSAAFRPCD IDTKIGN.AR |
| hrv-63 | ITSLPAMNRR FFLDLDIVVN DRYKDSQGKL NVSAAFQPCD VNAKVGN.AR |
| hrv-64 | ITSLSAMNRR FFLDLDIVVH ERYKDNQGKL DVPKSFKPCD VDNKIGN.AK |
| ef173417-64 | ITSLSAMNRR FFLDLDIVVH ERYKDNQGKL DVPKSFKPCD VDNKIGN.AK |
| hrv-65 | ITCTTAMNRR FFLDLDIVVK EVFKINQGRL NLQCALKPCK .DGKIGN.AR |
| hrv-66 | ITSLPAMNRR FFLDLDIVVC DKYKDSQGKL NVSAAFRPCD VNTKIGN.AR |
| hrv-67 | ITSLSAMNRR FYLDLDIVVN ENYRDTQGKL NVPKSFKPCD IDNKIGN.AK |
| hrv-68 | ITCTTAMNRR FFLDLDIVVK DKYKLSQGKL NLQCALKPCN .EGKIGN.AX |
| hrv-71 | ITCTPAMNRR FFLDLDIIVK DDYKINQGKL NLQCALKPCK .VGKVGN.AK |
| dq473492-73 | ITSLQAMNRR FFLDLDIVVH DNYKDTQGKL DVSKAFKPCD VGTKIGN.AR |
| dq473494-74 | ITSLSAMNRR FFLDLDIVVH DNYRDNQGKL YVPKSFKPCD IDNKIGN.AK |
| dq473510-75 | ITSLPAMNRR FFLDLDIVVH DNYKDIQGKL DVPKAFKLCD IDTKVGN.AR |
| dq473502-76 | ITSLPAMNRR FYLDLDIVVS EKYKDQQGKL NVAAAFQPCD VNVKIGN.AR |
| hrv-76 | ITSLPAMNRR FYLDLDIVVS EKYKDQQGKL NVAAAFQPCD VNVKIGN.AR |
| hrv-77 | ITSLPAMNRR FFLDLDIVVC DKYKDSQGKL NVSAAFQLCD VNTKVGN.AR |
| hrv-78 | ISSLPAMNRR FFLDLDIEVS ENYKDGNGRI QLDCALKPCN DNIKIGN.AK |
| ef173418-78 | ISSLPAMNRR FFLDLDIEVS ENYKDGNGRI QLDCALKPCN DNIKIGN.AK |
| hrv-80 | ITCTTAMNRR FFLDLDITVK EEYKIQQGKL NLRCALKPCK .EGKVGN.AK |
| hrv-81 | ISSLPAMNRR FYLDLDITVH DNFKDNQGKL DVSRAFQPCD TNLKIGN.AK |
| hrv-81-f06 | ISSLPAMNRR FYLDLDITVH DNFKDNQGKL DVSRAFQPCD TNLKIGN.AK |
| hrv-81-f07 | ISSLPAMNRR FYLDLDITVH DNFKDNQGKL DVSRAFQPCD TNLKIGN.AK |
| hrv-82 | ITSLSAMNRR FYLDLDIIVH ENYKNTQGKL DVSRSFKPCD VDTKIGN.AK |
| dq473509-82 | ITSLSAMNRR FYLDLDIIVH ENYKNAQGKL DVSRSFKPCD VDTKIGN.AK |
| hrv-85 | ITSLPAMNRR FFLDLDIIVC DKYKDAQGKL NVCAAFKPCD IDTKIGN.AK |
| pico-85 | ITSLPAMNRR FFLDLDIIVC DKYKDAQGKL NVCAAFKPCD IDTKIGN.AK |
| dq473504-88 | VSSLPAMSRR FYFDLDIQVK KEY.LMGGKL DIAKSFRPCD VDIKIGN.AK |
| a10937-89 | VSSLPAMARR FYFDLDIQVK KEY.LLDGKL DIAKSFRPCD VNIKIGN.AK |
| hrv-89 | VSSLPAMARR FYFDLDIQVK KEY.LLDGKL DIAKSFRPCD VNTKIGN.AK |
| hrv-89-f08 | VSSLPAMARR FYFDLDIQVK KEY.LLDGKL DIAKSFRPCD VNTKIGN.AK |
| hrv-89-f09 | VSSLPAMARR FYFDLDIQVK KEY.LLDGKL DIAKSFRPCD VNTKIGN.AK |
| m16248-89 | VSSLPAMARR FYFDLDIQVK KEY.LLDGKL DIAKSFRPCD VNIKIGN.AK |
| hrv-90 | ITSLPAMNRR FFLDLDIVVN DKYKDSQGKL NVSAAFQPCD VDAKIGN.AR |
| hrv-94 | ITSLSAMNRR FFLDLDIVVH ERYKDNQGKL DVPKSFKPCD VDNKIGN.AK |
| ef173419-94 | ITSLSAMNRR FFLDLDIVVH ERYKDNQGKL DVPKSFKPCD VDNKIGN.AK |
| hrv-95 | ISTLPALNRR LFLDLTIKVN PKY.LESGKL NLDCALKACD QEQKIGN.AR |
| hrv-96 | ITSLQAMNRR FFLDLDIVVH DNYKDAQGKL DVSKAFKPCD VXAKIGN.AR |
| hrv-98 | ITSLPAMNRR FFLDLDIIVC DKYKDSQGKL NVSAAFKPCD VDTKIGN.AK |
| hrv-100 | ITSLPAMNRR FFLDLDIIVC DKYKDSQGKL NVSAAFKPCD VDAKIGN.AR |
| ef186077-qpm | VSSPEAIDRR FFLDCDVKIM PAY.NTNNLL NVAKALLPCT DCPKPEFYKQ |
| ef077279-nat001 | VSTPEAIDRR FFLDCDVKIM PAY.NTNNLL NVAKALLPCT DCPKPEFYKQ |
| ny1078 | VSSPEAIDRR FFLDCDVKIM PAY.NTNNLL NVAKALLPCT DCPKPEFYKQ |
| ef582385-c024 | VTVPEAIDRR FYLDLDLKIL QGYQNQVGLL DTAKALQPCA NCSKPPHYKQ |
| ef077280-nat045 | IQTPEALERR FFLDMDVKVL SPYSSQRGLL DTAKALQPCT KCDKPTHYSR |
| ef582387-c026 | VSSPEAIDRR FFLDCDVKIM PAY.NTNNLL NVAKALLPCT DCPKPEFYKQ |
| ef582386-c025 | ITIPEAITRR FFLDVDLKIM PGYKTHAGLL DTAKALQACS DCAKPLHYKQ |
| dq473485-03 | ILNPEALIRR FGFDLDICMH STY.TKNGKL NAAMATSLCK DCHQPSNFKK |
| ef173422-03 | ILNPEALIRR FGFDLDICLH STY.TKNGKL NAAMATSLCK DCHQPSNFKK |
| dq473490-04 | ILNPEALARR FGFDLDMCLH TTF.TRSGKL NAALASELCK DCHKPANFKK |
| hrv-05 | VLNPEALARR FGFDLDMCLH TTY.TKSGKL NTALASELCK DCHKPTNFKR |
| dq473486-06 | ILNPEALARR FGFDLDICLH STY.TKNGKL NATMSTSLCK DCHQPANFKK |
| 105355-14 | ILNPEALVRR FGFDLDICLH TTY.TKNGKL NAGMSTKTCK DCHQPSNFKK |
| k02121-14 | ILNPEALVRR FGFDLDICLH TTY.TKNGKL NAGMSTKTCK DCHQPSNFKK |
| ef173420-17 | ILNPEALARR FGFDLDICLH TTY.TKSGKL NVAMATELCK DCYKPSNFNK |
| hrv-26 | ILNPEALARR FGFDLDMCLH STF.TKSGKL NAALASELCK DCHKPTNFKR |
| hrv-27 | VLNPEALARR FGFDLDMCLH SSF.TKSGKL NTAMACELCK DCHKPTNFKK |
| ef173421-27 | VLNPEALARR FGFDLDMCLH SSF.TKSGKL NTAMACELCK DCHKPTNFKK |
| dq473487-35 | ILNPEALARR FGFDLDICLH STY.TKNGKL NVTMATSICK DCHQPSNFKK |
| hrv-35 | ILNPEALARR FGFDLDICLH STY.TKNGKL NVTMATSICK DCHQPSNFKK |
| ef173423-37 | ILNPEALARR FGFDLDICLH STY.TKNGKL NTTMSTSICK DCHQPSNFKK |
| hrv-42 | VLNPEALARR FGFDLDMCLH TTY.TKSGKL NAALASELCK DCHKPTNFKR |
| dq473488-48 | ILNPEALARR FGFDLDICLH TTY.TKSGKL NAAMATELCK DCHKPSNFNK |
| hrv-52 | ILNPEALARR FGFDLDICLH TTY.TKSGKL NAAMATELCK DCHKPSNFSK |
| hrv-52-f10 | ILNPEALARR FGFDLDICLH TTY.TKSGKL NVAMATELCK DCHKPSNFSR |
| ef173424-52 | ILNPEALARR FGFDLDICLH TTY.TKSGKL NAAMATELCK DCHKPSNFSK |
| hrv-69 | ILNPEALVRR FGFDLDICLH TSF.TKSGKL NVAMATELCK DCHKPSNFNR |
| dq473489-70 | ILNPEALARR FGFDLDICLH TTY.TKSGKL NAAMATELCK DCHKPSNFNK |
| hrv-72 | ILNPDALARR FGFDLDICLH STY.TKNGKL NAAMATRVCK DCHQPTNFKK |
| hrv-79 | ILNPEALVRR FGFDLDICLH TTF.TKNGKL NAAMATSICK DCHQPSNFKK |
| hrv-83 | ILNPEALVRR FGFDLDICLH STY.TKNGKL NAAMATSICK DCHQPSNFKK |
| hrv-84 | VLNPEALARR FGFDLDMCLH SSY.TKSGKL NVAMACELCK DCHTPTNFKK |
| hrv-86 | ILNPEALTRR FGFDLDICLH STY.TKNGKL NASMATSLCK DCHKPSNFKK |
| hrv-91 | ILNPEALVRR FGFDLDICLH TTY.TKSGKL NAAMATELCK DCHKPSNFNK |
| hrv-92 | ILNPEALARR FGFDLDICLH STY.TKNGKL NAAMVTTICK DCHQPTNFKK |
| ef173425-93 | VLNPEALARR FGFDLDMCLH SSF.TKSGKL NAAMACELCK DCHKPTNFKK |
| hrv-97 | VLNPEALARR FGFDLDMCLH SSF.TKSGKL NAAMACELCK DCHKPTNFKK |
| hrv-99 | ILNPEALARR FGFDLDMCLH TTY.TRSGKL NAALASELCK DCHKPTNFKR |
| | 1451 1500 |
| annotation | .......... .......... .......... .......... ........QG |
| af499637-cva13 | CCPLVCGKAI QLMDKYSRQR FSVDEITTLV INEKNRRANI GNCMEALFQG |
| af546702-cva21 | CCPLVCGKAL QLMDRYTRQR FTVDEITTLI MNEKNRRANI GNCMEALFQG |
| v01149-pv1m | CCPLVCGKAI QLMDKSSRVR YSIDQITTMI INERNRRSNI GNCMEALFQG |
| d00239-01 | CCPFVCGKAV SFKDRSTCST YTLAQVYNHI LEEDKRRRQV VDVMSAIFQG |
| pico-01 | CCPFVCGKAV TFKDRSTCST YTLAQVYNHI LEEDKRRRQV VDVMSAIFQG |
| hrv-01 | CCPFVCGKAV XFKDRSTCST YTLAQVYNHI LEEDKRRRQV VDVMSAIFQG |
| x02316-02 | CCPFVCGKAV SFKDRNSCNK YSLAQVYNIM IEEDRRRRQV VDVMTAIFQG |
| hrv-07 | CCPFICGKAV EFKDRNTCAV LSLSRLYNHI REEDRRRSSA AQAMEAIFQG |
| dq473503-07 | CCPFICGKAV EFKDRNTCAV LSLSRLYNHI REEDRRRSSA AQAMEAIFQG |
| hrv-08 | CCPLICGKAV SFVNRNNNEE LSLSRVYNQI VHEHNRRLNV SKHMEAIFQG |
| pico-09 | CCPFICGKAV VFKDRNTCQT YTLCQIYNMI IEEDKRRRNV IDVMTAIFQG |
| hrv-09 | CCPFICGKAV VFKDRNTCQT YTLCQIYNMI IEEDKRRRNV IDVMTAIFQG |
| hrv-09-f01 | CCPFICGKAV VFKDRNTCQT YTLCQIYNMI IEEDKRRRNV IDVMTAIFQG |
| hrv-09-f02 | CCPFICGKAV VFKDRNTCQT YTLCQIYNMI IEEDKRRRNV IDVMTAIFQG |
| dq473498-10 | CCPFICGKAV MFRDRNTCQT YTLAQVYNRI LDEDKRRRQV IDVMAAIFQG |
| hrv-10 | CCPFICGKAV MFRDRNTCQT YTLAQVYNRI LDEDKRRRQV IDVMAAIFQG |
| ef173414-11 | CCPFICGKAV MFKDRNTCQS YTLAQVYNRI LDEDKRRRQV VDVMSAIFQG |
| ef173415-12 | CCPLICGKAI TFRDRNTNES YALSTIYNKI VEENKNRKML SKSLEAIFQG |
| hrv-13 | CCPFICGKAV TFKDRNTCQS YPLSQIYNLI LEEDKRRTHV VDVMTAIFQG |
| hrv-13-f03 | CCPFICGKAV TFKDRNTCQS YPLSQIYNLI LQEDKRRTHV VDVMTAIFQG |
| dq473493-15 | CCPFICGKAV TFKDRNNCQA YSLSRIYNMI MEEDKRRRNV VDVMTAIFQG |
| l24917-16 | CCPFVCGKAV TFKDRNTCRT YSLSQIYNQI LEEDKRRRQV VDVMSAIFQG |
| hrv-18 | CCPFICGKAV IFRDRNTCQT YTLSQVYNLI LEEDRKRTRV VDVMAAIFQG |
| hrv-19 | CCPFICGRAV SFKDRNTCRE YSLSQIYNVI MEEDKRRRNV VDVMSAIFQG |
| hrv-20 | CCPLICGKAL QFRDRNTGEH LSLATVYNRI TQESKNRKEL TNSLQAIFQG |
| hrv-21 | CCPFICGKAV MFKDRNTCRT YTLAQIYNQI LEEDKRRRQV IDVMSAIFQG |
| hrv-22 | CCPFICGKAV SFKDRNTCQE YSLSXIYNMI IEEDKRRRNV VDVMTAIFQG |
| dq473497-23 | CCPFVCGKAV SFKDRNSCNK YTLAQIYNIM LEEDRRRRQM VDVMSAIFQG |
| hrv-24 | CCPFICGKAV MFKDRNTCTS YTLSQVYNNI LDEDKRRRQV VDVMSAIFQG |
| ef173416-24 | CCPFICGKAV MFKDRNTCTS YTLSQVYNNI LDEDKRRRQV VDVMSAIFQG |
| hrv-25 | CCPFICGKAV VFRDRNTCQT YTLAQVYNQI LVEDKRRRQV IDVMAAIFQG |
| dq473508-28 | CCPLICGKAL QFKDRNSGDY LSLATVYNRI TQESKNRKDL TNSLQAIFQG |
| hrv-29 | CCPFICGKAV NFRDRNTCQT YTLAQVYNQI LAEDKRRRRV IDVMAAIFQG |
| dq473512-30 | CCPFVCGKAV SFKDRNTCNK YTLAQIYNIM LEEDQRRRQM VDVMSAIFQG |
| hrv-30 | CCPFVCGKAV SFKDRNTCNK YTLAQIYNIM LEEDQRRRQM VDVMSAIFQG |
| hrv-31 | CCPFICGKAV IFRDRNNCQT YTLAQVYNQI LAEDKRRRQV VDVMAAIFQG |
| hrv-32 | CCPFICGKAV VFKDRNTCQT YTLCQIYNMI IEEDKRRRSV VDVMSAIFQG |
| hrv-33 | CCPFICGKAV MFKDRNTCQS YTLAQVYNHI LEEDRRRRQV VDVMSAIFQG |
| hrv-34 | CCPFICGKAV NFRDRNTCQT YTLSQVYNQM LEEDKRRRQV IDVMAAIFQG |
| dq473501-34 | CCPFICGKAV NFRDRNTCQT YTLSQVYNQM LEEDKRRRQV IDVMAAIFQG |
| dq473505-36 | CCPFICGKAV EFKDRNSCTT LSLSQLYNHI KEEDRRRSSA AQAMEAIFQG |
| hrv-38 | CCPFICGKAV SFKDRNTCQT YSLSQIYNMV IEEDKRRRNV VDVMSAIFQG |
| dq473495-38 | CCPFICGKAV SFKDRNTCQT YSLSQIYNMI IEEDKRRRNV VDVMSAIFQG |
| ay751783-39 | CCPFVCGKAV SFKDRNSCNK YTLAQIYNIM LEEDKRRRQV IDVMSAIFQG |
| hrv-40 | CCPFICGKAV IFRDRNTCQT YTLAQVYNLI LDEDKRRRQV IDVMAAIFQG |
| dq473491-41 | CCPFICGKAV TFKDRNTCLN YSLSQIYNLI LEEDKRRTHV VDVMSAIFQG |
| hrv-43 | CCPFICGKAV SFKDRNTCQS YALCQIYNKV LEEDKRRRNV VDVMSAIFQG |
| dq473499-44 | CCPFICGKAV NFRDRNTCQT YTLAQVYNQI LAEDKRRRQV VDVMAAIFQG |
| hrv-45 | CCPLICGKAI TFINRHNNEE LTLSQIYNQV VNEHNRRLNV SKHMEAIFQG |
| dq473506-46 | CCPLICGKAL QFRDRSNGEH LSLATIYNRI THESKNRKEL TNSLQAIFQG |
| hrv-47 | CCPFICGKAV IFRDRNNCQT YTLAQVYNRI LVEDKRRRQV VDVMAAIFQG |
| dq473496-49 | CCPFVCGKAF SFKDRNSCNK YTLAQVYNIM LEEDKRRRQV VDVMSAIFQG |
| hrv-49-f04 | CCPFVCGKAV SFKDRNTCNK YTLAQVYNIM LEEDKRRRQV VDVMSAIFQG |
| hrv-50 | CCPFICGKAV NFRDRNTCQT YTLAQVYNQM LEEDKRRRXV IDVMTAIFQG |
| hrv-51 | CCPLICGKAV VFRDRNTGDY LSLSTIYNRI TEESRNRKEL NTSLQAIFQG |
| dq473507-53 | CCPLICGRAL QFRDRSNGEH LSLATIYNRI TQESKNRKEL TNSLQAIFQG |
| hrv-54 | CCPFICGKAV VFRDRNTCQT YTLSQVYNQI LDEDKRRRQV VDVMAAIFQG |
| hrv-54-f05 | CCPFICGKAV MFRDRNTCQT YTLSQVYNQI LDEDKRRRQV VDVMAAIFQG |
| dq473511-55 | CCPFICGKAV MFKDRNTCHT YALAQVYNQI LDEDKRRRQV IDVMSAIFQG |
| hrv-56 | CCPFICGKAV MFRDRNTCQT YTLAQVYNQI LEEDRRRRQV IDVMAAIFQG |
| hrv-57 | CCPFICGKAV TFKDRNTCXT FSLAQVYNQI LDEDKRRRQV IDVMSAIFQG |
| hrv-58 | CCPFICGKAV EFKDRNTCNV LSLSQLYNHI REEDRRRSSA AQAMEAIFQG |
| dq473500-59 | CCPFICGKAV TFRDRNTCQN YTLSQVYNQM LEEDKRRRQV VDVMAAIFQG |
| hrv-60 | CCPFICGKAV SFKDRNTCQV YPLSQIYNLI IEEDKRRRNV VDVMSAIFQG |
| hrv-61 | CCPFICGKAV TFKDRNTCLS YPLSQIYNLI LQEDKRRTHV VDVMSAIFQG |
| hrv-62 | CCPFICGKAV IFRDRNTCQT YTLAQVYNQI LAEDKRRRQV IDVMAAIFQG |
| hrv-63 | CCPFICGKAV TFRDRNTCQN YTLSQVYNHM LEEDKRRRQV VDVMAAIFQG |
| hrv-64 | CCPFICGKAV SFRDRNTCQE YSLCQIYNII IEEDKRRRNV VDVMTAIFQG |
| ef173417-64 | CCPFICGKAV SFRDRNTCQE YSLCQIYNII IEEDKRRRNV VDVMTAIFQG |
| hrv-65 | CCPLICGKAV VFKDRNTGDY LSLATVYNKI TEENRNRKEL NTSLQAIFQG |
| hrv-66 | CCPFICGKAV VFRDRNTCQT YTLAQVYNLM LEEDRKRTQV IDVMAAIFQG |
| hrv-67 | CCPFICGKAV VFKDRNTCQT YTLCQIYNMI IEEDKRRRNV IDVMTAIFQG |
| hrv-68 | CCPLICGKAL QFRDRNNGDH LSLATIYNRI TQESKNRKDL TNSLQAIFQG |
| hrv-71 | CCPLICGKAV LFRDRNTGEH LSLATIYNKI VEESKNRKEL NTSLQAIFQG |
| dq473492-73 | CCPFICGKAV TFKDRNTCLS YSLSQIYNLI LQEDKRRTHV VDVMTAIFQG |
| dq473494-74 | CCPFICGKAV TFKDRNNCQI YSLSQIYNMI IEEDKRRRNV IDVMTAIFQG |
| dq473510-75 | CCPFICGKAV TFKDRNTCQS YTLSQIYNKI LEEDKRRRNV VDVMIAIFQG |
| dq473502-76 | CCPFICGKAV MFKDRNTCQS YTLAQVYNHI LNEDKRRRQV VDVMSAIFQG |
| hrv-76 | CCPFICGKAV MFKDRNTCQS YTLAQVYNHI LNEDKRRRQV VDVMSAIFQG |
| hrv-77 | CCPFICGKAV VFRDRNTCQT YTLSQVYNLI LEENRKRTQV VDVMAAIFQG |
| hrv-78 | CCPFVCGKAI TFRDRNSCEK YTLARIYHKI LEEDKRRQCL SNALEAVFQG |
| ef173418-78 | CCPFVCGKAI TFRDRNSCEK YTLARIYHKI LEEDKRRQCL SNALEAVFQG |
| hrv-80 | CCPLICGKAL VFRDRNTGEH LSLATIYNRI VEESKNRKEL TNSLQAIFQG |
| hrv-81 | CCPFVCGKAV MFKDRNTCRT YSLSQVYNQI LEEDKRRRQV VDVMSAIFQG |
| hrv-81-f06 | CCPFVCGKAV MFKDRNTCKT YSLSQVYSQI LEEDKRRRQV IDVMSAIFQG |
| hrv-81-f07 | CCPFVCGKAV MFKDRNTCKT YSLSQVYSQI LEEDKRRRQV IDVMSAIFQG |
| hrv-82 | CCPFICGKAV SFKDRNTCEV YSLCRIYNMI MQEDRHRRSV VDVMTAIFQG |
| dq473509-82 | CCPFICGKAV SFKDRNTCEV YSLCQIYNMI MQEDRHRRSV VDVMTAIFQG |
| hrv-85 | CCPFICGKAV IFRDRNTCQT YTLAQVYNLI LDEDKRRRQV IDVMAAIFQG |
| pico-85 | CCPFICGKAV IFRDRNTCQT YTLAQVYNLI LDEDKRRRQV IDVMAAIFQG |
| dq473504-88 | CCPFICGKAV EFKDRNTCAT LSLSQLYNHI REEDRRRSSA ARAMEAIFQG |
| a10937-89 | CCPFICGKAV EFKDRNSCTT LSLSQLYSHI KEEDRRRSSA AQAMEAIFQG |
| hrv-89 | CCPFICGKAV EFKDRNSCTT LSLSQLYSHI KEEDRRRSSA AQAMEAIFQG |
| hrv-89-f08 | CCPFICGKAV EFKDRNSCTT LSLSQLYSHI KEEDRRRSSA AQAMEAIFQG |
| hrv-89-f09 | CCPFICGKAV EFKDRNSCTT LSLSQLYSHI KEEDRRRSSA AQAMEAIFQG |
| m16248-89 | CCPFICGKAV EFKDRNSCTT LSLSQLYSHI KEEDRRRSSA AQAMEAIFQG |
| hrv-90 | CCPFVCGKAV MFKDRNTCXS YTLAQVYNTI LDEDKRRRRV IDVMSAIFQG |
| hrv-94 | CCPFICGXAV SFRDRNTCQE YSLSQIYNMI IEEDKRRRNV VDVMTAIFQG |
| ef173419-94 | CCPFICGKAV SFRDRNTCQE YSLSQIYNMI IEEDKRRRNV VDVMTAIFQG |
| hrv-95 | CCPLICGKAV SFVNRNNNEE LSLSRVYNQI VHEHNRRLNV SKHMEAIFQG |
| hrv-96 | CCPFICGKAV TFKDRNTCLS YPLSQIYNLI LQEDKRRTHV VDVMSAIFQG |
| hrv-98 | CCPFVCGKAV VFRDRNTCQT YTLSQVYNQI LYEDKRRRQV VDVMAAIFQG |
| hrv-100 | CCPFICGKAV VFRDRNTCQS YTLAQVYNQI LVEDKRRRQV IDVMAAIFQG |
| ef186077-qpm | CCPLTCGKAL ILQNKRTKAS YSINTVVQQL RQEKKNRKCV EHNLTAIFQG |
| ef077279-nat001 | CCPLTCGKAL ILQNKRTKAS YSINTVVQQL RQEKKNRKCV EYNLTAIFQG |
| ny1078 | CCPLICGKAL ILQNKRTKAS YSINTVVQQL RQEKKNRKCV EHNLTAIFQG |
| ef582385-c024 | CCPLLCGKAV VLVNRRTKGS YAINMVVQQL IEESKSRKSV GNNLTAIFQG |
| ef077280-nat045 | CCPLLCGKAV VLVNRKTQVS YSVNMIVEQL RTEYGNRRSV KTNLAAIFQG |
| ef582387-c026 | CCPLTCGKAL ILQNKRTKAS YSINTVVQQL RQEKKNRKCV EHNLTAIFQG |
| ef582386-c025 | CCPLLCGKAI ILQNRRTSAS LSINMVVSQL REESTTRKRV HTNLNAIFQG |
| dq473485-03 | CCPLVCGKAI SLVDRVSNVR FSIDQLVTAI INDYKNKVKI TDSLEVLFQG |
| ef173422-03 | CCPLVCGKAV SLVDRVSNVR FSIDQLVTAI INDYKNKVKI TDSLEVLFQG |
| dq473490-04 | CCPLICGKAL SLVDRHTNVR YSIDQLVTAI ITEHVNKTKI SNSLEVLFQG |
| hrv-05 | CCPLICGKAL SLVDRRTNVR YSIDQLVTAI ITEHANKTKI SSSLEALFQG |
| dq473486-06 | CCPLVCGKAI SLVDRTTNIR YSVDQLVTAI INDYKNKMKI TDSLEVLFQG |
| l05355-14 | CCPLVCGKAI SLVDRTTNVR YSVDQLVTAI ISDFKSKMQI TDSLETLFQG |
| k02121-14 | CCPLVCGKAI SLVDRTTNVR YSVDQLVTAI ISDFKSKMQI TDSLETLFQG |
| ef173420-17 | CCPLICGKAI SLVDRSSNVR YSLDQLVTAI ITDFNNKLKI TDSLEVLFQG |
| hrv-26 | CCPLICGKAL SLVDRRTNVR YSIDQLVTAI ITEHVNKTKI SSSLEVLFQG |
| hrv-27 | CCPLICGKAL SLVDRHTNVR YSIDQLVTAI LQEHNNKTKI TNSLEILFQG |
| ef173421-27 | CCPLICGKAL SLVDRHTNVR YSIDQLVTAI LQEHNNKTKI TNSLEILFQG |
| dq473487-35 | CCPLVCGKAI SLVDRTSNVR YSVDQLVTAI INDYRNKVKI TDSLEVLFQG |
| hrv-35 | CCPLVCGKAI SLVDRTSNVR YSVDQLVTAI INDYRNKVKI TDSLEVLFQG |
| ef173423-37 | CCPLVCGKAI SLVDRATNIR YSVDQLVTAI INDYKNKIKI TDSLEALFQG |
| hrv-42 | CCPLICGKAL SLVDRRTNVR FSIDQLVTAI ITEHANKTKI SSSLEVLFQG |
| dq473488-48 | CCPLICGKAI SLVDRSTNVR YSIDQLVTAI ISDFNNKLKI TDSLEILFQG |
| hrv-52 | CCPLICGKAV SLVDRSTNVR YSLDQLVTAI ITDYNNKLKI TDSLEVLFQG |
| hrv-52-f10 | CCPLICGKAI SLVDRSTNVR YSLDQLVTAI ITDYNNKLKI TDSLEVLFQG |
| ef173424-52 | CCPLICGKAV SLVDRSTNVR YSLXQLVTAI ITDYNNKLKI TDSLEVLFQG |
| hrv-69 | CCPLICGKAI SLVDRSTNVR YSLDQLVTAI ITDFNNKLKI TDSLEILFQG |
| dq473489-70 | CCPLICGKAI SLVDRTSNVR YSLDQLVTAI ITDFNNKLKI TDSLEILFQG |
| hrv-72 | CCPLVCGKAT SLVDRATNVR YSLDQLVTAV VNDYKNKMQI TDSLETLFQG |
| hrv-79 | CCPLVCGKAV SLVDRATNIR YSVDQLVTAI INDYKNKMKI TDSLEALFQG |
| hrv-83 | CCPLVCGKAT SLVDRATNIR YSVDQLVTAI ISDYKNKVKI TDSLEILFQG |
| hrv-84 | CCPLICGKAL SLVDKHTNVR YTIDQLVTAI LQEHNNKTKI TSSLEVLFQG |
| hrv-86 | CCPLVCGKAI SLVDRATNVR YSVDQLITAI INDYNNKIKI TDSLETLFQG |
| hrv-91 | CCPLICGKAI SLVDRTSNVR YSLDQLVTAI ITDFNNKLKI TDSLEVLFQG |
| hrv-92 | CCPLVCGKAI SLVDRATNIR YSVDQLVSAI INDYRNKIKI TDSLEALFQG |
| ef173425-93 | CCPLICGKAL SLVDRHTNVR YSIDQLVTAI LQEHNNKTKI TNSLEVLFQG |
| hrv-97 | CCPLICGKAL SLVDRHTNVR YSIDQLVTAI LQEHNNKTKI TNSLEVLFQG |
| hrv-99 | CCPLICGKAL SLVDRRTNVR YSIDQLVTAI ITEHANKTKI SSSLEVLFQG |
| | 1501 1550 |
| annotation | P......... .........H HHHHHHHHHH HHHHHHHHHH .......... |
| af499637-cva13 | PLEFKDLKID IKTSPPPECI NDLLQSVDSP EVREYCEKKG WIVNVSSETT |
| af546702-cva21 | PLRYKDLKID VKTVPPPECI SDLLQAVDSQ EVRDYCEKKG WIVNVTSQIQ |
| v01149-pv1m | PLQYKDLKID IKTSPPPECI NDLLQAVDSQ EVRDYCEKKG WIVNITSQVQ |
| d00239-01 | P.....ISLD ...APPPPAI ADLLQSVRTP EVIKYCQDNK WIVPAEC..Q |
| pico-01 | P.....ISLD ...VPPPPAI ADLLQSVRTP EVIKYCQDNK WVIPAEC..Q |
| hrv-01 | P.....ISLD ...XPPPPAI XDLLQSVRTP EVIKYCQDNK WVIPAEC..Q |
| x02316-02 | P.....IDM. ..KNPPPPAI TDLLQSVRTP EVIKYCEGNR WIIPAEC..K |
| hrv-07 | ......IDL. ..HTPPPPAI TDLLRSVRTP EVVKYCQDNN WIVPAEC..T |
| dq473503-07 | ......IDL. ..HTPPPPAI TDLLRSVRTP EVVKYCQDNN WIVPAEC..T |
| hrv-08 | P.....INMQ ...SPPPPAI VDLLRSTRNE EVINYCKNQN WIIPADV..S |
| pico-09 | P.....ITM. ..NLPPPPAI ADLLQSVRTP EVVKYCEDNK WTIPAEC..K |
| hrv-09 | P.....ITM. ..NSPPPPAI ADLLQSVRTP EVVKYCEYNK WIIPAEC..K |
| hrv-09-f01 | P.....ITM. ..NSPPPPAI ADLLQSVRTP EVVKYCEDNK WIIPAEC..K |
| hrv-09-f02 | P.....ITM. ..NSPPPPAI ADLLQSVRTP EVVKYCEDNK WIIPAEC..K |
| dq473498-10 | P.....ITLE ...GPPPAAI SDLLQSVRTP EVIKYCEGNK WIIPAEC..K |
| hrv-10 | P.....ITLE ...GPPPAAI SDLLQSVRTP EVIKYCEGNK WIIPAEC..K |
| ef173414-11 | P.....ISLE ...RPPPAAI SDLLQSVRTP EVIKYCEGNK WIIPAEC..K |
| ef173415-12 | P.....ISL. ..KNPPPPAI SDLLNAVRTP EVIKYCEENN WTVPAQC..T |
| hrv-13 | P.....ISME ...VPPPPAI TDLLRSVKTP EVIKYCEDNK WIIPAEC..K |
| hrv-13-f03 | P.....ISME ...APPPPAI ADLLRSVKTP EVIKYCEDNK WIIPADC..R |
| dq473493-15 | P.....ISM. ..NNPPPPAI VDLLQSVRTP EVVKYCEDNK WMLPADC..K |
| l24917-16 | P.....ISMD ...KPPPPAI TDLLRSVRTP EVIKYCQDNK WIVPADC..Q |
| hrv-18 | P.....ITLE ...GPPPAAI SDLLQSVRTP EVIKYCEENK WIIPAEC..K |
| hrv-19 | P.....ISM. ..NAPPPPAI TDLLQSVRTP EVIKYCEDNK WXIPADC..K |
| hrv-20 | P.....IDIV ..NKPPPPAI IDLLESVRSP DVIKYCEENK WIIPSDC..K |
| hrv-21 | P.....ISLE ...GPPPAAI SDLLQSVRTP EVIKYCEANK WIIPAEC..K |
| hrv-22 | P.....ISMD ...XPPPPAI XDLLQSVRTP EVIKYCEXNK WIIPAEC..K |
| dq473497-23 | P.....IDM. ..KNPPPPAI TDLLQSVRTP EVIKYCEENR WIIPAEC..K |
| hrv-24 | P.....ITLE ...GPPPAAI NDLLKSVRTP EVIKYCEYNK WTVPAEC..K |
| ef173416-24 | P.....ITLE ...GPPPAAI NDLLKSVRTP EVIKYCEDNK WTVPAEC..K |
| hrv-25 | P.....ITIE ...GPPPAAI NDLLKSVRTP EVIRYCEENK WIVPAEC..K |
| dq473508-28 | P.....IDVV ..NKPPPPAI IDLLKSVRNP DVIRYCEENK WIIPSDC..K |
| hrv-29 | P.....ITLE ...GPPPAAI NDLLKSVRTP EVIKYCEENK WIIPTEC..K |
| dq473512-30 | P.....IDM. ..RNPPPPAI TDLLQAVRTP EVIKYCEENK WVIPAEC..K |
| hrv-30 | P.....IDM. ..RNPPPPAI TDLLQAVRTP EVIKYCEENK WIIPAEC..K |
| hrv-31 | P.....ITLE ...GPPPAAI NDLLKSVRTP EVIKYCEDNK WIVPTEC..K |
| hrv-32 | P.....ISM. ..NSPPPPAI ADLLQSVRTP EVVKYCEYNK WIIPAEC..K |
| hrv-33 | P.....ISLE ...XPPPAAI SDLLQSVRTP EVIKYCEDNK WIIPAGC..R |
| hrv-34 | P.....ISME ...GPPPAAI SDLLQSVRTP EVIKYCEXNK WMIPAEC..K |
| dq473501-34 | P.....ISME ...GPPPAAI SDLLQSVRTP EVIKYCEVNK WMIPAEC..K |
| dq473505-36 | ......IDLQ ...SPPPPAI ADLLRSVKTP EIIKYCQGNN WIVPAEC..S |
| hrv-38 | P.....ISM. ..NAPPPPAI EDLLQSVRTP EVIKYCEENK WIIPADC..K |
| dq473495-38 | P.....ISM. ..NAPPPPAI EDLLQSVRTP EVIKYCEENK WIIPADC..K |
| ay751783-39 | P.....ISLQ ...NPPPPAI ADLLQSVRTP EVIKYCEENK WIIPAEC..K |
| hrv-40 | P.....ISLE ...GPPPAAI SDLLQSVRTP EVIKYCEDNK WIIPAGC..K |
| dq473491-41 | P.....ISME V...PPPPAI TDLLRSVKTP EVIKYCENNK WIIPAEC..K |
| hrv-43 | P.....ITLD ...APPPPAI TDLLRSVRTP EVIKYCEDNK WMIPAGC..K |
| dq473499-44 | P.....ITLE ...GPPPAAI NDLLKSVRTP EVIKYCEGNK WIIPAEC..K |
| hrv-45 | P.....IDMQ ...APPPPAI VDLLRSTRNE DVINYCKNQN WIIPADI..S |
| dq473506-46 | P.....IDVV ..NKPPPPAI VDLLKSVRNP DVIRYCEENK WIIPADC..R |
| hrv-47 | P.....ITLE ...GPPPAAI NDLLKSVRTP EVIKYCEDNK WVVPAEC..K |
| dq473496-49 | P.....IDM. ..KNPPPPAI TDLLQSVRTP EVIKYCEVNR WIIPAEC..K |
| hrv-49-f04 | P.....IDM. ..KNPPPPAI TDLLQSVRTP EVIKYCEDNR WIIPAEC..K |
| hrv-50 | P.....ISLE ...GPPPAAI SDLLHSVRTP EVIKYCEDNK WIVPAEC..K |
| hrv-51 | P.....IDVI ..NKPPPPAI LDLLKSVRNP DVIKYCEENK WMIPADC..K |
| dq473507-53 | P.....IDIV ..NKPPPPAI VDLLKSVRNP DVIRYCEENK WIIPADC..K |
| hrv-54 | P.....ISLE ...GPPPAAI SDLLQSVRTP EVIKYCEYNK WIIPAEC..K |
| hrv-54-f05 | P.....ISLE ...GPPPAAI SDLLQSVRTP EVIKYCEDNK WIIPAEC..K |
| dq473511-55 | P.....ISL. ..KGPPPAAI SDLLQSVRTP EVIKYCEENK WIIPTEC..K |
| hrv-56 | P.....ITLE ...GPPPAAI SDLLQSVRTP EVIKYCENNK WIIPAEC..K |
| hrv-57 | P.....ISLE ...GPPPAAI SDLLQSVRTP EVIKYCEQNK WIIPAEC..K |
| hrv-58 | ......IDLQ ...SPPPPAI TDLLRSVRTP EIIKYCQGNN WIVPAEC..S |
| dq473500-59 | P.....ITLE ...GPPPAAI NDLLQSVRTP EVIKYCEYNK WIIPAEC..K |
| hrv-60 | P.....ISM. ..NAPPPPAI ADLLQSVRTP EVIKYCEENK WVIPADC..K |
| hrv-61 | P.....ISME ...VPPPPAI TDLLRSVKTP EVIKYCEDNK WTIPADC..K |
| hrv-62 | P.....ITIE ...GPPPAAI NDLLKSVRTP EVIRYCEENK WMVPAEC..K |
| hrv-63 | P.....ITLE ...GPPPAAI SDLLQSVRTP EVIKYCEKNK WIIPAEC..K |
| hrv-64 | P.....ISMS ...APPPPAI ADLLQSVRTP EVIKYCEENK WIIPADC..K |
| ef173417-64 | P.....ISMS ...APPPPAI ADLLQSVRTP EVIKYCEENK WIIPADC..K |
| hrv-65 | P.....IDVV ..NKPPPPAI LDLLKSVRNP DVIKYCEDNK WIVPADC..K |
| hrv-66 | P.....ITLE ...GPPPAAI SDLLQSVRTP EVIKYCEGNK WIIPAEC..K |
| hrv-67 | P.....ISM. ..NLPPPPAI TDLLQSVRTP EVVKYCEDNN WIIPAEC..K |
| hrv-68 | P.....IDVV ..NKPPPPAI IDLLRSVRNP DVIKYCEENK WMIPSDC..K |
| hrv-71 | P.....IDVV ..NKPPPPAI LDLLKSVRNP EVIRYCEDNR WIVPVDC..R |
| dq473492-73 | P.....ISME ...VPPPPAI TDLLRSVRTP EVIKYCENNK WIVPADC..K |
| dq473494-74 | P.....ISM. ..NNPPPPAI ADLLQSVRTP EVIKYCEDNK WIIPAEC..K |
| dq473510-75 | P.....ITLD ...APPPPAI TDLLRSVRTP EVIKYCEENK WIIPTDC..R |
| dq473502-76 | P.....ISLE ...GPPPAAI SDLLQSVRTP EVIKYCEYNK WIVPAEC..R |
| hrv-76 | P.....ISLE ...GPPPAAI SDLLQSVRTP EVIKYCEYNK WIVPAEC..R |
| hrv-77 | P.....ITLE ...GPPPAAI NDLLXSVRTP EVIKYCEDNK WIIPXEC..K |
| hrv-78 | P.....ITL. ..NGPPPPAI LDLLKSVRTP EVIKYCEDNK WIVPAEN..T |
| ef173418-78 | P.....ITL. ..NGPPPPAI LDLLKSVRTP EVIKYCEDNK WIVPAEN..T |
| hrv-80 | P.....IDM. ..NRPPPPAI VDLLKSVRNP DVIKYCEDNK WVVPTEC..K |
| hrv-81 | P.....ISMD ...KPPPPAI TDLLRSVRTP EVIKYCQDNK WIVPADC..Q |
| hrv-81-f06 | P.....ISMD ...KPPPPAI TDLLRSVRTP EVIKYCQDNK WIVPADC..Q |
| hrv-81-f07 | P.....ISMD ...KPPPPAI TDLLRSVRTP EVIKYCQDNK WIVPADC..Q |
| hrv-82 | P.....ITMD ...APPPPAI ADLLQSVRTP EVIKYCEENK WIIPADC..K |
| dq473509-82 | P.....ITM. ..NAPPPPAI ADLLQSVRTP EVIKYCEENK WIIPADC..K |
| hrv-85 | P.....ISLV ...GPPPAAI SDLLQSVRTP EVIRYCEVNK WIIPAEC..K |
| pico-85 | P.....ISLV ...GPPPAAI SDLLQSVRTP EVIRYCEVNK WIIPAEC..K |
| dq473504-88 | ......IDLQ ...SPPPPAI TDLLRSVRTP EIIKYCQDNN WIVPAEC..S |
| a10937-89 | ......IDLQ ...SPPPPAI ADLLRSVKTP EIIKYCQDNN WIVPAEC..S |
| hrv-89 | ......IDLQ ...SPPPPAI ADLLRSVKTP EIIKYCQDNN WIVPAEC..S |
| hrv-89-f08 | ......IDLQ ...SPPPPAI ADLLRSVKTP EIIKYCQDNN WIVPAEC..S |
| hrv-89-f09 | ......IDLQ ...SPPPPAI ADLLRSVKTP EIIKYCQDNN WIVPAEC..S |
| m16248-89 | ......IDLQ ...SPPPPAI ADLLRSVKTP EIIKYCQDNN WIVPAEC..S |
| hrv-90 | P.....ITLE ...GPPPAAI NDLLKSVRTP EVIKYCEGNK WIVPAEC..K |
| hrv-94 | P.....ISM. ..NAPPPPAI ADLLQSVRTP EVIKYCEENK WVIPADC..K |
| ef173419-94 | P.....ISM. ..NAPPPPAI ADLLQSVRTP EVIKYCEENK WVIPADC..K |
| hrv-95 | P.....INMQ ...SPPPPAI VDLLRSTRNE EVINYCKNQN WIIPADV..S |
| hrv-96 | P.....ISME ...VPPPPAI TDLLRSVKTP EVIKYCENNK WIIPADC..K |
| hrv-98 | P.....ISLE ...GPPPAAI SDLLQSVRTP EVIKYCEYNK WIIPAEC..K |
| hrv-100 | P.....ISLD ...GPPPAAI SDLLQSVRTP EVIKYCENNR WIIPAEC..K |
| ef186077-qpm | .......LGD DST...PGFI IDLLSASKDP KVIEFCEKEG WIKQSRC.SK |
| ef077279-nat001 | .......LGD DST...PGFI VDLLSASKDP KVIEFCEKEG WIKQSKH.SK |
| ny1078 | .......LGD DTT...PGFI IDLLSASKDP KVIEFCEKEG WIKQSKC.SK |
| ef582385-c024 | .......LGD KET...PGFI IDLLSSSKDP KVIQYCQDQG YISPVHG..S |
| ef077280-nat045 | .......LGD QET...PGFI IDLLRSSKDP KVIQYCEEEG LILRSST..T |
| ef582387-c026 | .......LGD DST...PGFI VDLLSASKDP KVIEFCEKEG WIKQSKY.SK |
| ef582386-c025 | .......LGS DET...PGFI VDLLSSSRDP RVIEYCEKEG LIVKATH..T |
| dq473485-03 | PV.YKDLEID ICNTPPPECI SDLLKSVDSE EVREYCKKKK WIIPQIS.TN |
| ef173422-03 | PV.YKDLEID ICNTPPPECI SDLLKSVDSE EVREYCKKKK WIIPQIS.TN |
| dq473490-04 | PT.YKDLNIN VTTTPPPECI VDLLKSVDNE EVRQYCQGKK WLIPEIP.TN |
| hrv-05 | PV.YKDLSID ITTTPPPECI VDLLKSVDNE EVRQYCQEKR WLIPEIP.TN |
| dq473486-06 | PV.YKDLEID VCNTPPPECI NDLLKSVDSE EVREYCKKKK WIIPQIP.TN |
| l05355-14 | PV.YKDLEID VCNTPPPECI NDLLKSVDSE EIREYCKKKK WIIPEIP.TN |
| k02121-14 | PV.YKDLEID VCNTPPSECI NDLLKSVDSE EIREYCKKKK WIIPEIP.TN |
| ef173420-17 | PV.YKDLEID VHNTPPPECI SDLLKSVDSE ELREYCKQKN WLIPEIP.TN |
| hrv-26 | PV.YKDLNID VTTTPPPECI VDLLKSVDNE EVRQYCQEKR WLIPELP.TN |
| hrv-27 | PV.YKDMTID VYDKPPPDCI VDLLKSVDSV EVRQYCREKK WLIPEIP.TN |
| ef173421-27 | PV.YKDMTID VYDKPPPDCI VDLLKSVDSV EVRQYCREKK WLIPEIP.TN |
| dq473487-35 | PV.YKDLEID VCNTPPPECI NDLLKSVDSE EVRDYCKKKN WIIPQIP.TN |
| hrv-35 | PV.YKDLEID VCNTPPPECI NDLLKSVDSE EVRDYCKKKN WIIPQIP.TN |
| ef173423-37 | PV.YRDLEID VCNKSPPECI NDLLKSVDSE ELREYCKKKN WIIPQVP.TN |
| hrv-42 | PT.YKDLHID VTTTPPPECI VDLLKSVDNE EVRQYCQEKR WLIPEIP.TN |
| dq473488-48 | PT.YKDLEIE IHNTPPPECI SDLLKSVDSE ELREYCKQKN WLIPEIP.TN |
| hrv-52 | PV.YKDLEID VHSTPPPECI NDLLKSVDSK ELREYCKQKN WLIPEIP.TN |
| hrv-52-f10 | PV.YKDLEID VHSTPPPECI NDLLKSVDSE ELREYCKQKN WLIPEIP.TN |
| ef173424-52 | PV.YKDLEID VHSTPPPECI NDLLKSVDSK ELREYCKQKN WLIPEIP.TN |
| hrv-69 | PV.YKDLEID VHTTPPPECI NDLLKSVDSE ELREYCKQKN WLIPEIP.TN |
| dq473489-70 | PV.YKDLEID VHNTPPPECI SDLLKSVDSE ELREYCKQKN WLIPEIP.TN |
| hrv-72 | PV.YKDLEID VNSTPPPECI NDLLRSVDSE EIREYCKKKN WTIPEIP.TN |
| hrv-79 | PV.YKDLEID ICNTPPPECI NDLLKSVDSE EIREYCKKKN WTIPQIP.TN |
| hrv-83 | PV.YKDLEID VCNTPPPECI SDLLRSVDSE EVREYCKKKN WIIPQIP.TN |
| hrv-84 | PV.YKDLNID VHDKPPPECI VDLLKSVDCD EVRQYCKEKK WLIPEIP.TN |
| hrv-86 | PV.YKDIEID VCNTPPPECI NDLLKSVDSE EVREYCKKKK WTIPEIP.TD |
| hrv-91 | PV.YKDLEID IHSTPPPECI SDLLKSVDSE ELREYCKQKN WLIPEIP.TN |
| hrv-92 | PV.YKDLEID VCNTPAPECI NDLLKSVDSE EVREYCKKKN WIIPQIP.TN |
| ef173425-93 | PV.YKDLTID VNDKPPPDCI VDLLKSVDSE EVRQYCREKK WLIPEVP.TN |
| hrv-97 | PV.YKDITID VHDKPPPDCI VDLLKSVDSE EVRQYCREKK WLIPEIP.TN |
| hrv-99 | PV.YKDLSID ITTTPPPECI VDLLKSVDNE EVRQYCQEKK WLIPEIP.TN |
| | 1551 1600 |
| annotation | .......... .......... .......... .....QGP.. .......... |
| af499637-cva13 | IERNLSRAMT ILQALTTFAA VAGVVYVMYR LFAGQQGAYT GLP.NKRPNV |
| af546702-cva21 | LERNINRAMT ILQAVTTFAA VAGVVYVMYK LFAGQQGAYT GLP.NKKPNV |
| v01149-pv1m | TERNINRAMT ILQAVTTFAA VAGVVYVMYK LFAGHQGAYT GLP.NKKPNV |
| d00239-01 | IERDLSIANS IITIIANIIS IAGIIFVIYK LFCTLQGPYS GEP.KPKTKM |
| pico-01 | VERDLNIANS IIAIIANIIS IAGIIFVIYK LFCSLQGPYS GEP.KPKTKV |
| hrv-01 | VERDLNIANS IIAIIANIIS IAGIIFVIYK LFCSLQGPYS GEP.KPKTKV |
| x02316-02 | IEKELNLANT IITIIANVIG MARIIYVIYK LFCTLQGPYS GEP.KPKTKI |
| hrv-07 | IERDLGLANL TISIIANVVS IVGIIYIIYK LFCTLQGPYS GEP.KPKTRA |
| dq473503-07 | IERDLGLANL TISIIANVVS IVGIIYIIYK LFCTLQGPYS GEP.KPKTRA |
| hrv-08 | VERELNLVNL SISILANIIS IVGIIYIIYK LFISLQGPYS GLP.SKKKTI |
| pico-09 | IERDLNLANN IITIIANIIS IVGIIFVIYK LFCSLQGPYS GEP.KPKTRV |
| hrv-09 | IERDLNLANN IITIIANIIS IAGIIFVIYK LFCSLQGPYS GEP.KPKTRI |
| hrv-09-f01 | IERDLNLANN IITIIANIIS IAGIIFVIYK LFCSLQGPYS GEP.KPKTRV |
| hrv-09-f02 | IERDLNLANN IITIIANIIS IAGIIFVIYK LFCSLQGPYS GEP.KPKTRV |
| dq473498-10 | VERDLNIANT IITIIANIIS ISGIIYVIYK LFCTLQGPYS GEP.KPKTKI |
| hrv-10 | VERDLNIANT IITIIANIIS ISGIIYVIYK LFCTLQGPYS GEP.KPKTKI |
| ef173414-11 | IERDLNIANT IITIIANVIS ISGIIYVIYK LFCTLQGPYS GEP.KQKTKI |
| ef173415-12 | IERELNYCNL VLSMIANLVS IIGIVYVIYK LFCSFQGPYS GQP.EPNTRQ |
| hrv-13 | IERDLNLANN IIAIIANIIS IAGIIYIIYK LFCSFQGPYS GEP.KPKTKI |
| hrv-13-f03 | IERDLNLANN IITIIANIIS IAGIIYIIYK LFCSFQGPYS GEP.KPKTKI |
| dq473493-15 | IERDLNLANS IITIIANIIS IAGIIFVIYK LFCSLQGPYS GEP.KPKTKI |
| l24917-16 | IERDLNIANS IITIIANIIS IAGIIYIIYK LFCSLQGPYS GEP.KPKTKV |
| hrv-18 | VERDLNIANT IITIIANIVS ISGIIYVIYK LFCTLQGPYS GEP.KPKTKI |
| hrv-19 | IERDLNIANN IITIIANIIS IAGIIFVIYK LFCSLQGPYS GEP.KPKTKV |
| hrv-20 | LERDLNYANL IISMLANVIS IMGVIYIIYK LFCSLQGPYS GEP.KPTTRK |
| hrv-21 | VERDLNIANT IITIIANIIS ISGIIYVIYK LFCTLQGPYS GEP.KPKTKM |
| hrv-22 | IERDLNLANN IITIIANVIS IAGIIFVIYK LFCTLQGPYS GEP.KPKTKI |
| dq473497-23 | IEKELNLANT IITIIANVIS IAGIIYVIYK LFCTLQGPYS GEP.KIKTKI |
| hrv-24 | IERDLNVANT IITIMANIIS IAGIIYVIYK LFCTLQGPYS GEP.IKKTKV |
| ef173416-24 | IERDLNVANT IITIMANIIS IAGIIYVIYK LFCTLQGPYS GEP.IKKTKV |
| hrv-25 | IERDLNIANT IITIIANIIS ISGIIYVIYK LFCTLQGPYS GEP.KPKTKI |
| dq473508-28 | LERDLNYANL IISMLANVIS IMGVIYIIYK LFCSLQGPYS GEP.KPITKK |
| hrv-29 | VERDLNLANT IISIIANIIS ISGIIYVIYK LFCTLQGPYS GEP.KPKTKV |
| dq473512-30 | IEKGLNLANT IITIIANVIS IAGIIYVIYK LFCTLQGPYS GEP.KPKTKI |
| hrv-30 | IEKGLNLANT IITIIANVIS IAGIIYVIYK LFCTLQGPYS GEP.KPKTKI |
| hrv-31 | IERDLNIANT IITIIANIIS ISGIIYVIYK LFCTLQGPYS GEP.KPKTKI |
| hrv-32 | IERDLNLANN IITIIANIIS IAGIIFVIYK LFCSLQGPYS GEP.KPKTKI |
| hrv-33 | IERDLNIANT IITIIANVIS ISGIIYVIYK LFCTLQGPYS GEP.KQRTKI |
| hrv-34 | VERDLNIANT IITIIANIIS ISGIIYVIYK LFCTLQGPYS GEP.KPKTKV |
| dq473501-34 | VERDLNIANT IITIIANIIS ISGIIYVIYK LFCTLQGPYS GEP.KPKTKV |
| dq473505-36 | IERDLGIANM TIGIIANVVS IVGVIYIIYK LFCTLQGPYS GEP.KPKSKA |
| hrv-38 | IERDLNLANN IITIIANIIS IAGIIFVIYK LFCSLQGPYS GEP.KPKTRT |
| dq473495-38 | IERDLNLANN IITIIANIIS IAGIIFVIYK LFCSLQGPYS GEP.KPKTRT |
| ay751783-39 | IEKELNLANT IITIIANVIN IAGIIYVIYK LFCTLQGPYS GEP.KPKTKI |
| hrv-40 | VERDLNIANT IITIIANIIS ISGIIYVIYK LFCTLQGPYS GEP.KPKTKI |
| dq473491-41 | IERDLNLANN IITIIANIIS IAGIIYIIYK LFCSFQGPYS GEP.KPKTRV |
| hrv-43 | IERDLNLANN IITIIANIIS IAGIIFVIYK LFCSLQGPYS GEP.KPKTKV |
| dq473499-44 | IERDLNLANT IITIIANIIS ISGIIYVIYK LFCTLQGPYS GEP.KPKTKI |
| hrv-45 | IERELNLVNL SISILANLIS VIGIIYIIYK LFVSLQGPYS GLP.TKKKVI |
| dq473506-46 | LERDLNYANL IISMIANVIS IMGVIYIIYK LFCSLQGPYS GEP.KPVTKK |
| hrv-47 | VERDLNIANT VITIIANIIS ISGIIYVIYK LFCTLQGPYS GEP.KPKTKI |
| dq473496-49 | IEKELNLANT IITIIANVIS IAGIIYVIYK LFCTLQGPYS GEP.KPKTKV |
| hrv-49-f04 | IEKELNLANT IITIIANVIS IAGIIYVIYK LFCTLQGPYS GEP.KPKTKV |
| hrv-50 | IERDLNIANT IITIIANIIS ISGIIYVIYK LFCTLQGPYS GEP.KPKTRM |
| hrv-51 | LERDLNYANL VISMIANVIS ILGVIYIIYK LFCSLQGPYS GEP.KPISRK |
| dq473507-53 | LERDLNYANV IISMIANVIS IMGVIYIIYK LFCSLQGPYS GEP.KPVTKK |
| hrv-54 | IERDLNLANT IITIIANIIS ISGIIYVIYK LFCTLQGPYS GEP.KPKTKV |
| hrv-54-f05 | IERDLNLANT IITIIANIIS ISGIIYVIYK LFCTLQGPYS GEP.KPKTKV |
| dq473511-55 | VERDLNIANT IITIIANIIS ISGIIYVIYK LFCTLQGPYS GEP.KPKTRM |
| hrv-56 | IERDLNIANT IITIIANIIS ISGIIYVIYK LFCTLQGPYS GEP.KPKTKI |
| hrv-57 | IERDLNVANT IITIIANIIS ISGIIYVIYK LFCTLQGPYS GEP.KPKTRM |
| hrv-58 | IERDLGIANM AISIIANVVS IVGVIYIIYK LFCTLQGPYS GEP.KPKSRA |
| dq473500-59 | VERDLNIANT IITIIANIIS ISGIIYVIYK LFCTLQGPYS GEP.KPKTKI |
| hrv-60 | IERDLNLANN ITTIIANIIS IAGIIFVIYK LFCSLQGPYS GEP.KPKTKV |
| hrv-61 | IERDLILANN IITIIANIIS IAGIIYIIYK LFCSFQGPYS GEP.KPKTKI |
| hrv-62 | VERDLNIANT IITIIANIIS ISGIIYVIYK LFCTLQGPYS GEP.RPKTKI |
| hrv-63 | VERDLNIANT IITIIANIIS ISGIIYVIYK LFCTLQGPYS GEP.KPKTKI |
| hrv-64 | IERDLNLANN ITTIIANVIS IAGIIFVIYK LFCTLQGPYS GEP.KPKTKV |
| ef173417-64 | IERDLNLANN ITTIIANVIS IAGIIFVIYK LFCTLQGPYS GEP.KPKTKV |
| hrv-65 | LERDLNYANL VISMIANVVS ILGVIYIIYK LFCSLQGPYS GEP.KPATRK |
| hrv-66 | VERDLNIANT IITIIANIIS ISGIIYVIYK LFCTLQGPYS GEP.KPKTRI |
| hrv-67 | IERDLNLANN IIAIIANIIS IAGIIFVIYK LFCSLQGPYS GEP.KPKTKI |
| hrv-68 | LERDLNYANL IISMLANVIS IMGVIYIIYK LFCSLQGPYS GEP.KPVTKK |
| hrv-71 | LERDLNYANL VISMIANVIS ILGVIYIIYK LFCSLQGPYS GEP.KPITRK |
| dq473492-73 | IERDLNLANN IITIIANIIS IAGIIYIIYK LFCSFQGPYS GEP.KPKTKV |
| dq473494-74 | IERDLNLANN IITIIANIIS IAGIILVIYK LFCSLQGPYS GEP.KPKPKI |
| dq473510-75 | IERDLNLANN IITIIANVVS IAGIICVIYK IFCSLQGPYS GEP.KPKTKI |
| dq473502-76 | IERDLNIANT IITIIANVIS ISGIIYVIYK LFCTLQGPYS GEP.KQKTKI |
| hrv-76 | IERDLNIANT IITIIANVIS ISGIIYVIYK LFCTLQGPYS GEP.KQKTKI |
| hrv-77 | VERDLNMANT IITIIANIIS ISGIIYVIYK LFCTLQGPYS GEP.KPKTKV |
| hrv-78 | IERDLNVANS VITIIANLIS IVGVIYIIYK LFCSLQGPYS GEP.KPKTKT |
| ef173418-78 | IERDLNVANS VITIIANLIS IVGVIYIIYK LFCSLQGPYS GEP.KPKTKT |
| hrv-80 | LERDLNYANL VISMLANVIS IVGVIYIIYK LFCSLQGPYS GEP.KPVTKK |
| hrv-81 | IERDLNIANN IITIIANIIS IAGIIYIIYK LFCTLQGPYS GEP.KPKTKI |
| hrv-81-f06 | IERDLSIANS IITIIANIVS IAGIIYIIYK LFCTLQGPYS GEP.KPKTKV |
| hrv-81-f07 | IERDLSIANS IITIIANIIS IAGIIYIIYK LFCTLQGPYS GEP.KPKTKV |
| hrv-82 | IERDLNLANN IITIIANVIS IAGIIFVIYK LFYTLQGPYS GEP.KPKPKI |
| dq473509-82 | IERDLNLANN IITIIANVIS IAGIIFVIYK LFCTLQGPYS GEP.KPKPKI |
| hrv-85 | VERDLNIANT IITIIANIIS ISGIIYVIYK LFCTLQGPYS GEP.KPKTKI |
| pico-85 | VERDLNIANT IITIIANIIS ISGIIYVIYK LFCTLQGPYS GEP.KPKTKI |
| dq473504-88 | IERDLGLANL TIGIIANVVS IVGIIYIIYK LFCTLQGPYS GEP.KPKTRA |
| a10937-89 | IERDLGIANM TIGIIANVVS IVGVIYIIYK LFCTLQGPYS GEP.KPKSRA |
| hrv-89 | IERDLGIANM TIGIIANVVS IVGVIYIIYK LFCTLQGPYS GEP.KPKSRA |
| hrv-89-f08 | IERDLGIANM TIGIIANVVS IVGVIYIIYK LFCTLQGPYS GEP.KPKSRA |
| hrv-89-f09 | IERDLGIANM TIGIIANVVS IVGVIYIIYK LFCTLQGPYS GEP.KPKSRA |
| m16248-89 | IERDLGIANM TIGIIANVVS IVGVIYIIYK LFCTLQGPYS GEP.KPKSRA |
| hrv-90 | IERDLNVANT IITIIANIIS ISGIIYVIYK LFCTLQGPYS GEP.VKKTKI |
| hrv-94 | IERDLNLANN IITIIANVIS IAGIIFVIYK LFCTLQGPYS GEP.KPKTKT |
| ef173419-94 | IERDLNLANN IITIIANVIS IAGIIFVIYK LFCTLQGPYS GEP.KPKTKT |
| hrv-95 | VERELNLVNL SISILANIIS IVGIIYIIYK LFISLQGPYS GLP.SKKKTI |
| hrv-96 | IERDLNLANN IITIIANIIS IAGIIYIIYK LFCSFQGPYS GEP.KPKTKI |
| hrv-98 | IERDLNIANT IITIIANIIS ISGIIYVIYK LFCTLQGPYS GEP.KPKTKI |
| hrv-100 | VERDLNIANT IITIIANIIS ISGIIYVIYK LFCTLQGPYS GEP.KPKTKV |
| ef186077-qpm | IERDFNYAQY CINCVGSIIL ILGTVYALYK LMCIAQGPYT GLP.QPKVRK |
| ef077279-nat001 | IERDFNYVQY CLNCVGSIIL ILGTVYALYK LMCIAQGPYT GLP.QPKPRK |
| ny1078 | IERDFNYAQY CINCVGSIVL ILGTVYALYK LMCIAQGPYT GLP.QPKPRK |
| ef582385-c024 | IERDFAYVTY ALNCLGSLVI ILGTIYALYR LMCIAQGPYT GMP.TTPKKK |
| ef077280-nat045 | LDRHIDRVQQ AINCIGSLVV ILGTIYALYK LMSMTQGAYT GHV.EPTLKR |
| ef582387-c026 | IERDFNYAQY CINCVGSIVL ILGTVYALYK LMCIAQGPYT GLP.QPKPRK |
| ef582386-c025 | LEKNLEYTQY CLNCLGSLIL IIGTIYAIYK LLCVSQGAYS GNA.NPIPRK |
| dq473485-03 | IERAVNQASM IINTILMFVS TLGIVYVIYK LFAQTQGPYS GNPVHNKLKP |
| ef173422-03 | IERAVNQASM IINTILMFVS TLGIVYVIYK LFAQTQGPYS GNPVHNKLKP |
| dq473490-04 | VERAVNQATN LINTILMFIT TLGIIYMIYK LFAQTQGPYS GLDGKSKLKP |
| hrv-05 | IDRAVNQATS LINTILMFIT TLGIVYMIYK LFAQTQGPYS GLDNKSKLKP |
| dq473486-06 | IERAMNQASM IINTILMFVS TLGIVYVIYK LFAQTQGPYS GNPSHNKLKP |
| l05355-14 | IERAMNQASM IINTILMFVS TLGIVYVIYK LFAQTQGPYS GNPPHNKLKA |
| k02121-14 | IERAMNQASM IINTILMFVS TLGIVYVIYK LFAQTQGPYS GNPPHNKLKA |
| ef173420-17 | IERAVNQAST IINTILMFVS TLGILYVIYK LFAQTQGPYS GNPSHTKLKP |
| hrv-26 | IDRAVNQATS LINTILMFIT TLGIVYMIYK LFAQTQGPYS GLDSKSKLKP |
| hrv-27 | IDRAVNQATS LINTILMFIT TMGIVYMIYK MFAQTQGPYS GLDSKAKLKA |
| ef173421-27 | IDRAVNQATS LINTILMFIT TMGIVYMIYK MFAQTQGPYS GLDSKAKLKA |
| dq473487-35 | IERAVNQASM IINTILMFVS TLGIVYVIYK LFAQTQGPYS GNPTHNKLKP |
| hrv-35 | IERAVNQASM IINTILMFVS TLGIVYVIYK LFAQTQGPYS GNPTHNKLKP |
| ef173423-37 | IERAMNQASM IINTILMFVS TLGIVYVIYK LFAQTQGPYS GNPSHNKLKP |
| hrv-42 | VERAVNQATN LINTILMFIT TLGIIYMIYK LFAQTQGPYS GLDNKSKLKP |
| dq473488-48 | IERAVNQAST IINTILMFVS TLGILYVIYK LFAQTQGPYS GNPSHTKLKP |
| hrv-52 | IERAVNQAST IINTILMFVS TLGILYVIYK LFAQTQGPYS GNPSHTKLKP |
| hrv-52-f10 | IERAVNQAST IINTILMFVS TLGILYVIYK LFAQTQGPYS GNPSHTKLKP |
| ef173424-52 | IERAVNQAST IINTILMFVS TLGILYVIYK LFAQTQGPYS GNPSHTKLKP |
| hrv-69 | VERAVNQAST IINTILMFVS TLGILYVIYK LFAQTQGPYS GNPSHTKLKP |
| dq473489-70 | IERAVNQAST IINTILMFVS TLGILYVIYK LFAQTQGPYS GNPSHVKLKP |
| hrv-72 | IERAVNQASM IINTILMFVS TLGIIYVIYK LFAQTQGPYS GNPAHSKLKA |
| hrv-79 | IDRAMNQASM IINTILMFVS TLGIVYVIYK LFAQTQGPYS GNPSHNKLKP |
| hrv-83 | IERAVNQASM IINTILMFVS TLGIVYVIYK LFAQTQGPYS GNPSHGKLKA |
| hrv-84 | VERAVNQATS LINTILMFIT TMGIIYMIYK MFAQMQGPYS GLESKTKLKA |
| hrv-86 | IERAVNQASM IINTILMFVS TLGIVYVIYK LFAQTQGPYS GNVAHNKLKP |
| hrv-91 | IERAVNQAST IINTILMFVS TLGILYVIYK LFAQTQGPYS GNPSHTKLKP |
| hrv-92 | IERAVNQASM IINTILMFVS TLGIVYVIYK LFAQTQGPYS GNPAHNKLKA |
| ef173425-93 | IDRAVNQATS LINTILMFIT TMGIVYMIYK MFAQTQGPYS GLDNKTKLKA |
| hrv-97 | IDRAVNQATS LINTILMFIT TMGIVYMIYK MFAQTQGPYS GLDTKTKLKA |
| hrv-99 | VERAVNQATN LINTILMFIT TLGIVYMIYK LFAQTQGPYS GLDSKSKLKP |
| | 1601 1650 |
| annotation | ........QG PHHHHHHHHH HHHBBBBB.. ..BBBBBBBB ..BBBB..H. |
| af499637-cva13 | PTIRTAKVQG PGFDYAVAMA KRNITTATTS KGEFTMLGVY DNVAILPTHA |
| af546702-cva21 | PTIRVAKVQG PGFDYAVAMA KRNIVTATTT KGEFTMLGVH DNVAILPTHA |
| v01149-pv1m | PTIRTAKVQG PGFDYAVAMA KRNIVTATTS KGEFTMLGVH DNVAILPTHA |
| d00239-01 | PE.RRVVAQG PEEEFGRSIL KNNTCVITTD NGKFTGLGIY DRTLIIPTHA |
| pico-01 | PE.RRIVAQG PEEEFGRSIL KNNTCVITTG NGKFTGLGIH DRILSHPTHA |
| hrv-01 | PE.RRVVAQG PEEEFGRSIL KNNTCVITTG NGKFTGLGIH DRILIIPTHA |
| x02316-02 | PE.RRVVTQG PEEEFGMSLI KHNSCVITTE NGKFTGLGVY DRFVVVPTHA |
| hrv-07 | PE.RRVVTQG PEEEFGRSLI KHNCCVVTTD KGKFTGLGIY NQIMVLPTHS |
| dq473503-07 | PE.RRVVTQG PEEEFGRSLI KHNCCVVTTD KGKFTGLGIY NQIMVLPTHS |
| hrv-08 | PE.KRIVVQG PTTEFGLSLI KHNTCVVETD NGKFTGLGIF DNVLVIPTHA |
| pico-09 | PE.RRVVAQG PEEEFGRSLI KHNTCVVTTD NGKFTGLGIY DRYMIVPTHA |
| hrv-09 | PE.RRVVAQG PEEEFGRSLI KHNTCVVTTD NGKFTGLGIY DRYMIVPTHA |
| hrv-09-f01 | PE.RRVVAQG PEEEFGRSLI KHNTCVVTTD NGKFTGLGIY DRYMIIPTHA |
| hrv-09-f02 | PE.RRVVAQG PEEEFGRSLI KHNTCVVTTD NGKFTGLGIY DRYMIIPTHA |
| dq473498-10 | PE.RRVVAQG PEEEFGRSLI KHNSCIVTTQ NGKFTGLGIY DRMLILPTHA |
| hrv-10 | PE.RRVVAQG PEEEFGRSLI KHNSCIVTTQ NGKFTGLGIY DRMLILPTHA |
| ef173414-11 | PE.RRVVAQG PEEEFGRSLI KHNSCVVTTQ NGKFTGLGIY DRTIVIPTHA |
| ef173415-12 | PE.RRVVIQG PVEEFGRSLI KHNTRIVTTK NGKFTGLGIY DNLMVIPTHA |
| hrv-13 | PE.RRVVAQG PEEEFGRSII KHNTCVVTTD NGKFTGLGIY DKLMILPTHA |
| hrv-13-f03 | PE.RRVVAQG PEEEFGRSII KHNTCVVTTD NGKFTGLGIY DKLMVLPTHA |
| dq473493-15 | PE.RRVVAQG PEEEFGRSLI KHNTCVVTTD NGKFTGLGIY DRYMIVPTHS |
| l24917-16 | PE.RRVVAQG PEEEFGMSII KNNTCVVTTT NGKFTGLGIY DRILILPTHA |
| hrv-18 | PE.RRVVAQG PEEEFGRSLI KHNSCIVTTQ NGKFTGLGIY DRTLIIPTHA |
| hrv-19 | PE.RRVVAQG PEEEFGRSLI KHNTCVVTTD NGKFTGLGIY DRVMILPTHA |
| hrv-20 | PE.RRVVTQG PQEEFGRSLI KHNTCVVTTD NGKFTGLGIY DNIMIIPTHA |
| hrv-21 | PE.RRVVAQG PEEEFGRSLI KHNSCVVTTQ NGKFTGLGIY DRTLIIPTHA |
| hrv-22 | PE.RRVVAQG PEEEFGRSLI KHNTCVVTTD NGKFTGLGIY DRVMILPTHA |
| dq473497-23 | PE.RRVVAQG PEGEFGMSLI KHNSCVVTTE NGKFTGLGIY DRTLIVPTHA |
| hrv-24 | PE.RRVVAQG PEEEFGRSLI KHNSCIVTTQ NGKFTGLGIY DRVLIIPTHA |
| ef173416-24 | PE.RRVVAQG PEEEFGRSLI KHNSCIVTTQ NGKFTGLGIY DRVLIIPTHA |
| hrv-25 | PE.RRVVAQG PEEEFGRSLI KHNSCVVSTQ NGKFTGLGIY DRTLILPTHA |
| dq473508-28 | PE.RRVVTQG PQEEFGRSLI KHNTCVVTTN NGKFTGLGIY DNVMIIPTHA |
| hrv-29 | PE.RRVVAQG PEEEFGRSLI KHNSCVVSTQ NGKFTGLGIY DRTLILPTHA |
| dq473512-30 | PE.RRVVAQG PEEEFGLSLI KHNSCVVTTE NGKFTGLGIH DRIMIVPTHA |
| hrv-30 | PE.RRVVAQG PEEEFGLSLI KHNSCVVTTE NGKFTGLGIH DRIMIVPTHA |
| hrv-31 | PE.RRVVAQG PEEEFGRSLI KHNSCIVTTQ NGKFTGLGIY DRTLVLPTHA |
| hrv-32 | PE.RRVVAQG PEEEFGRSLI KHNTCVITTD NGKFTGLGIY DKYMIIPTHA |
| hrv-33 | PE.RRVVAQG PEEEFGRSLI KHNSCVVTTQ NGKFTGLGIY DRTIIIPTHA |
| hrv-34 | PE.RRVVAQG PEEEFGRSLI KHNSCVVTTE NGKFTGLGIY DRTLIVPTHA |
| dq473501-34 | PE.RRVVAQG PEEEFGRSLI KHNSCVVTTE NGKFTGLGIY DRTLIVPTHA |
| dq473505-36 | PE.RRVVTQG PEEEFGRSLL KHNCCIVTTD KGKFTGLGIY DQVMVLPTHS |
| hrv-38 | PE.RRVVAQG PEEEFGRSLI KHNSCIVTTQ NGKFTGLGIY DRTLIIPTHA |
| dq473495-38 | PE.RRVVAQG PEEEFGRSLI KHNSCIVTTQ NGKFTGLGIY DRTLIIPTHA |
| ay751783-39 | PE.RRVVAQG PEEEFGRSLI KHNSCVVTTQ NGKFTGLGIY DRVMIIPTHA |
| hrv-40 | PE.RRVVAQG PEEEFGRSLI KHNSCVVTTS NGKFTGLGIY DRTLIIPTHA |
| dq473491-41 | PE.RRVVAQG PEEEFGRSLI KHDTCVVTTD NGKFTGLGIY DKLMVLPTHA |
| hrv-43 | PE.RRIVAQG PEEEFGRSLI KHNTCVVTTD NGKFTGLGIY DKLMILPTHA |
| dq473499-44 | PE.RRVVAQG PEEEFGRSLI KHNSCVVSTQ NGKFTGLGIY DRTLILPTHA |
| hrv-45 | PE.KRVVVQG PSTEFGLSLI KHNTCIVETE NGKFTGLGIY DNLLVIPTHA |
| dq473506-46 | PE.RRVVTQG PQEEFGRSLI KHNTCVVTTS NGKFTGLGIY DNVMIIPTHA |
| hrv-47 | PE.RRVVAQG PEEEFGRSLI KHNSCIVTTQ NGKFTGLGIY DRTLILPTHA |
| dq473496-49 | PE.RRVVAQG PEEEFGMSLI KHNSCVITTE NGKFTGLGIY DKFVVVPTHA |
| hrv-49-f04 | PE.RRVVAQG PEEEFGMSLI KHNSCVITTD NGKFTGLGIY DKFVVVPTHA |
| hrv-50 | PE.RRVVAQG PEEEFGRSLI KHNSCVVTTE NGKFTGLGIY DRTLIIPTHA |
| hrv-51 | PE.RRIVTQG PQEEFGRSLL KHNACIVTTE NGKFTGLGIY DNVMILPTHA |
| dq473507-53 | PE.RRVVTQG PQEEFGRSLM KHNTCVVTTN NGKFTGLGIY DNVMIIPTHA |
| hrv-54 | PE.RRVVAQG PEEEFGRSLI KHNSCVVTTS NGKFTGLGIY DRTLIVPTHA |
| hrv-54-f05 | PE.RRVVAQG PEEEFGRSLI KHNSCVVTTS NGKFTGLGIY DRTLIVPTHA |
| dq473511-55 | PE.RRVVAQG PEEEFGRSLI KHNSCVVTTS NGKFTGLGIY DRTLIVPTHA |
| hrv-56 | PE.RRVVAQG PEEEFGRSLI KHNSCIVTTQ NGKFTGLGIY DRVLILPTHA |
| hrv-57 | PE.RRVVAQG PEEEFGRSLI KHNSCVVTTQ NGKFTGLGIY DRVLIIPTHA |
| hrv-58 | PE.RRVVTQG PEEEFGRSLL KHNCCIVTTD KGKFTGLGIY DQVMVLPTHS |
| dq473500-59 | PE.RRVVAQG PEEEFGRSLI KHNSCVVTTQ NGKFTGLGIY DRTIIIPTHA |
| hrv-60 | PE.RRVVAQG PEEEFGRSLI KHNSCVVTTS NGKFTGLGIY DRTLIIPTHA |
| hrv-61 | PE.RRVVTQG PEEEFGRSLI KHNTCVVTTD NGKFTGLGIY DKLMILPTHA |
| hrv-62 | PE.RRVVAQG PEEEFGRSLI KHNSCVVSTQ NGKFTGLGIY DRTLILPTHA |
| hrv-63 | PE.RRVVAQG PEEEFGRSLI KHNSCIVTTQ NGKFTGLGIY DRVLILPTHA |
| hrv-64 | PE.RRVVAQG PEEEFGRSLI KHNTCVVTTE NGKFTGLGIY DKIMILPTHA |
| ef173417-64 | PE.RRVVAQG PEEEFGRSLI KHNTCVVTTE NGKFTGLGIY DKIMILPTHA |
| hrv-65 | PE.RRVVTQG PQEEFGRSLL KHNACIVTTD NGKFTGLGIY DNVMILPTHA |
| hrv-66 | PE.RRVVAQG PEEEFGRSLI KHNSCIVTTS NGKFTGLGIY DRTLIIPTHA |
| hrv-67 | PE.RRVVAQG PEEEFGRSLI KHNTCVVTTD NGKFTGLGIY DRYLIIPTHA |
| hrv-68 | PE.RRVVTQG PQEEFGRSLI KHNTCVVTTS NGKFTGLGIY DNIMIIPTHA |
| hrv-71 | PE.RRVVTQG PQEEFGRSLL KHNACIVTTE NGKFTGLGIY DNVMIIPTHA |
| dq473492-73 | PE.RRVVAQG PEEEFGRSLI KHNTCVVTTD NGKFTGLGIY DKLMILPTHA |
| dq473494-74 | PE.RRIVAQG PEEEFGRSLI KHNTCVVTTE NGKFTGLGIY DRIMIVPTHS |
| dq473510-75 | PE.RRIVAQG PEEEFGRSLI KHNTCVVTTE NGKFTGLGIY DKVMILPTHA |
| dq473502-76 | PE.RRVVAQG PEEEFGRSLI KHNSCVVTTQ NGKFTGLGIY DRTIIIPTHA |
| hrv-76 | PE.RRVVAQG PEEEFGRSLI KHNSCVVTTQ NGKFTGLGIY DRTIIIPTHA |
| hrv-77 | PE.RRVVAQG PEEEFGRSLI KHNSCVVTTS NGKFTGLGIY DRTLILPTHA |
| hrv-78 | PE.RRVVTQG PVEEFGRSLI KHNTCVVTTE NGKFTGLGVH DNVMIIPTHS |
| ef173418-78 | PE.RRVVTQG PVEEFGRSLI KHNTCVVTTE NGKFTGLGVH DNVMIIPTHS |
| hrv-80 | PE.RRVVTQG PQEEFGRSII KHNTCVLTTN NGKFTGLGIH DNFMVIPTHA |
| hrv-81 | PE.RRVVAQG PEEEFGMSII KNNTCVVTTA NGKFTGLGIY DKILIIPTHA |
| hrv-81-f06 | PE.RRVVAQG PEEEFGMSII KNNTCVVTTA NGKFTGLGIY DKILIIPTHA |
| hrv-81-f07 | PE.RRVVAQG PEEEFGMSII KNNTCVVTTA NGKFTGLGIY DKILIIPTHA |
| hrv-82 | PE.RRVVAQG PEEEFGRSLI KHNTCVVTTD NGKFTGLGIY DKTMIIPTHA |
| dq473509-82 | PE.RRVVAQG PEEEFGRSLI KHNTCVVTTE NGKFTGLGIY DKTMIIPTHA |
| hrv-85 | PE.RRVVAQG PEEEFGRSLI KHNSCIVTTS NGKFTGLGIY DRTLIIPTHA |
| pico-85 | PE.RRVVAQG PEEEFGRSLI KHNSCIVTTS NGKFTGLGIY DRTLIIPTHA |
| dq473504-88 | PE.RRVVTQG PEEEFGRSLL KHNCCTVTTD KGKFTGLGIY DQIMVLPTHS |
| a10937-89 | PE.RRVVTQG PEEEFGRSLL KHNCCVVTTD KGKFTGLGIY DQVMVLPTHS |
| hrv-89 | PE.RRVVTQG PEEEFGRSLL KHNCCVVTTD KGKFTGLGIY DQVMVLPTHS |
| hrv-89-f08 | PE.RRVVTQG PEEEFGRSLL KHNCCVVTTD KGKFTGLGIY DQVMVLPTHS |
| hrv-89-f09 | PE.RRVVTQG PEEEFGRSLL KHNCCVVTTD KGKFTGLGIY DQVMVLPTHS |
| m16248-89 | PE.RRVVTQG PEEEFGRSLL KHNCCVVTTD KGKFTGLGIY DQVMVLPTHS |
| hrv-90 | PE.RRVVAQG PEEEFGRSLI KHNSCVVTTQ NGKFTGLGIY DRVIIVPTHA |
| hrv-94 | PE.RRVVAQG PEEEFGRSLI KHNTCVVTTD NGKFTGLGIY DRVMILPTHA |
| ef173419-94 | PE.RRVVAQG PEEEFGRSLI KHNTCVVTTD NGKFTGLGIY DRVMILPTHA |
| hrv-95 | PE.KRIVVQG PTTEFGLSLI KHNTCVVETD NGKFTGLGIF DNVLVIPTHA |
| hrv-96 | PE.RRVVAQG PEEEFGRSLI KHNTCVVTTD NGKFTGLGIY DRLMILPTHA |
| hrv-98 | PE.RRVVAQG PEEEFGRSLI KHNSCVVTTS NGKFTGLGIY DRTLIIPTHA |
| hrv-100 | PE.RRVVAQG PEEEFGRSLI KHNSCVVTTN NGKFTGLGIY DRTLIIPTHA |
| ef186077-qpm | PELRKAVIQG PEHEFGMALI KRNCHIATTD RGDFNLLGVY DNCAVIPTHA |
| ef077279-nat001 | PELRKAVVQG PEHEFGMALI KRNCHIATTD RGGFNLLGIY DNCAVIPTHA |
| ny1078 | PELRRAVAQG PEHEFGMALL KRNCHIATTD RGDFNLLGIH DNCAVLPTHA |
| ef582385-c024 | PELRRATLQG PEHEFLNALI RRNCHIITTD KGEFNLLGIY SNCAVVPTHA |
| ef077280-nat045 | SELRKAVTQG PEHEFMYALL KRNCHIAETR KGEFNLLGIH SNCAVIPTHA |
| ef582387-c026 | PELRKAVIQG PEHEFGMALI KRNCHIATTD RGDFNLLGVY DNCAVIPTHA |
| ef582386-c025 | PELRKAVKQG PEHEFVHSLI RRNCHVATTS RGDFNLLGIH DNCAVLPTHA |
| dq473485-03 | PTLKPVVVQG PNTEFALSLL RKNILTITTE KGEFTSLGIH DRICVLPTHA |
| ef173422-03 | PTLKPVVVQG PNTEFALSLL RKNILTITTE KGEFTSLGIH DRICVLPTHA |
| dq473490-04 | PTLRAVVTQG PNTEFALSLL RKNIVTVTTS KGEFTGLGIY DRILVIPTHA |
| hrv-05 | PTLRAVVTQG PNTEFALSLL RKNIVTVTTD KGEFTGLGIY ERVLVIPTHA |
| dq473486-06 | PSLRPVVVQG PNTEFALSLL RKNIVTITTE KGEFTGLGIY DRVCVIPTHA |
| l05355-14 | PTLRPVVVQG PNTEFALSLL RKNIMTITTS KGEFTGLGIH DRVCVIPTHA |
| k02121-14 | PTLRPVVVQG PNTEFALSLL RKNIMTITTS KGEFTGLGIH DRVCVIPTHA |
| ef173420-17 | PTLRQVVVQG PNTEFALSLL RKNIVTITTT KGEFTALGVH DRVCVVPTHS |
| hrv-26 | PTLRAVVTQG PNTEFALSLL RKNIVTVTTD KGEFTGLGVY DRLLVIPTHA |
| hrv-27 | PVLRAVVTQG PNTEFALSLL RKNIVTITTA KGEFTALGIY DRICVIPTHS |
| ef173421-27 | PVLRAVVTQG PNTEFALSLL RKNIVTITTA KGEFTALGIY DRICVIPTHS |
| dq473487-35 | PTLRPVVAQG PNTEFALSLL RKNIVTITTE KGEFTGLGIY DQICVIPTHA |
| hrv-35 | PTLRPVVAQG PNTEFALSLL RKNIVTITTE KGEFTGLGIY DQICVIPTHA |
| ef173423-37 | PSLRPVMVQG PNTEFALSLL RKNIVTITTE KGEFTGLGIY DRVCVIPTHA |
| hrv-42 | PTLRAVVTQG PNTEFALSLL RKNIVTVTTD KGEFTGLGIH ERILVIPTHA |
| dq473488-48 | PTLRQVVVQG PNTEFALSLL RKNIVTITTS KGEFTALGIH DRVCVVPTHS |
| hrv-52 | PTLRQVVVQG PNTEFAMSLL RKNIVTVTTT KGEFTALGIH DRVCVVPTHS |
| hrv-52-f10 | PTLRQVVVQG PNTEFAMSLL RKNIVTVTTT KGEFTALGIH DRVCVVPTHS |
| ef173424-52 | PTLRQVVVQG PNTEFAMSLL RKNIVTVTTT KGEFTALGIH DRVCVVPTHS |
| hrv-69 | PTLRQVVVQG PNTEFAMSLL RKNIVTITTS KGEFTALGVH DRVCVVPTHS |
| dq473489-70 | PTLRQVVVQG PNTEFALSLL RKNIVTITTT KGEFTALGIH DRVCVVPTHS |
| hrv-72 | PTLRPVIVQG PNTEFALSLL RKNIVTITTD KGEFTGLGIY DHICVIPTHA |
| hrv-79 | PTLRPVIVQG PNTEFALSLL RKNVVTITTE KGEFTGLGVY DHVCVIPTHA |
| hrv-83 | PTLRPVIVQG PNTEFALSLL RKNIVTITTE KGEFTGLGVY DQICVLPTHA |
| hrv-84 | PTLRAVVTQG PNTEFALSLL RKNIITVTTS KGEFTSLGIY DRICVIPTHS |
| hrv-86 | PTLRPVVVQG PNTEFALSLL RKNIVTITTE KGEFTGLGIY DRVCVVPTHS |
| hrv-91 | PTLRQVVVQG PNTEFALSLL RKNIITITTA KGEFTALGIY DRVCVVPTHS |
| hrv-92 | PTLRPIVVQG PNTEFALSLL RKNIVTITTE KGEFTGLGVY DHVCVVPTHA |
| ef173425-93 | PVLRAVVTQG PNTEFALSLL RKNIITITTS KGEFTALGIY DRICVIPTHS |
| hrv-97 | PVLRAVVTQG PNTEFALSLL RKNIVTITTA KGEFTALGIY DRICVIPTHS |
| hrv-99 | PTIRAVMTQG PNTEFALSLL RKNIVTVTTN KGEFTGLGIY DKILVVPTHA |
| | 1651 1700 |
| annotation | ...BBBB... BBBBBBBBBB BBB...BBBE BBBBBBB... ...HHHHHH. |
| af499637-cva13 | APGETVMIDG KEVEIIDAKA LEDQAGTNLE ITIITLKRNE KFRDIRPHIP |
| af546702-cva21 | APGETIIIDG KEVEILDARA LEDQAGTNLE ITIITLKRNE KFRDIRSHIP |
| v01149-pv1m | SPGESIVIDG KEVEILDAKA LEDQAGTNLE ITIITLKRNE KFRDIRPHIP |
| d00239-01 | DPGREVQVNG IHTKVLDSYD LYNRDGVKLE ITVIQLDRNE KFRDIRKYIP |
| pico-01 | DPGREVQVMG VHTKVLDSYD LYNRDGVKLE ITVIQLDRNE KFRDIRKYIP |
| hrv-01 | DPGREVQVNG VHTKVLDSYD LYNRDGVKLE ITVIQLDRNE KFRDIRKYIP |
| x02316-02 | DPGKEIQVDG ITTKVIDSYD LYNKNGIKLE ITVLKLDRNE KFRDIRRYIP |
| hrv-07 | DPGSEILIDG VKVKILDSYD LHNHEGIKLE ITVIKLDRNE KFRDIRKYLP |
| dq473503-07 | DPGSEILIDG VKVKILDSYD LHNHEGIKLE ITVIKLDRNE KFRDIRKYLP |
| hrv-08 | DPGKSVNIDG VEVKVVDSYD LFNKEGVKLE ITVILLSRNE KFRDIRKYIP |
| pico-09 | DPGKTVQING IVTNVVDSYD LHNKDGINLE ITVIKLDRNE KFRDIRKYIP |
| hrv-09 | DPGKTVQING IVTNVVDSYD LHNKDGIKLE ITVIKLDRNE KFRDIRKYIP |
| hrv-09-f01 | DPGKTVQING IVTNVVDSYD LHNKDGIKLE ITVIKLDRNE KFRDIRKYIP |
| hrv-09-f02 | DPGKTVQING IVTNVVDSYD LHNKDGIKLE ITVIKLDRNE KFRDIRKYIP |
| dq473498-10 | DPGREVQIDG ITTKVEDSFD LYNKDGVKLE ITVLKLKRNE KFKDIRKYIP |
| hrv-10 | DPGREVQIDG ITTKVEDSFD LYNKDGVKLE ITVLKLKRNE KFKDIRKYIP |
| ef173414-11 | DPGEEVQIDG ITTKVEDSYD LYNKDGIKLE ITVLKLQRNE KFRDIRKYIP |
| ef173415-12 | DPEKVVQIDG INVNVTDSYD LCNKQGVKLE ITVIKLDQNE KFRDIRKFIP |
| hrv-13 | DPGKEVCING IATKVSDSYD LYNKQGVKLE ISAIVLDRNE KFRDIRKYIP |
| hrv-13-f03 | DPGKEVCING ITTKVSDSYD LYNKQGVKLE ITAIVLDRNE KFRDIRKYIP |
| dq473493-15 | DPGKVVQVDG INTNVLDAYD LHNKDGIKLE ITVIKLDRNE KFRDIRKYIP |
| l24917-16 | DPGSEIQVNG IHTKVLDSYD LFNKEGVKLE ITVLKLDRNE KFRDIRKYIP |
| hrv-18 | DPGKEVQIDG ITTEVQDSYD LYNKDGVKLE ITVLKLKRNE KFKDIRKYIP |
| hrv-19 | DPGKTLQING INTSVTDSYD LYNKDGIKLE ITVVKLDRNE KFRDIRKYIP |
| hrv-20 | DAGKEVEIDG IKTQVDDAYD LHNSQGIKLE ITVLKLHRNE KFRDIRKYIP |
| hrv-21 | DPGKEVQIDG IATKVEDSYD LFNKDGVKLE ITVLKLKRNE KFKDIRKYIP |
| hrv-22 | DPGKIIQVNG INTTVCDSYD LYNKNGIKLE ITVVKLDRNE KFRDIRKYIP |
| dq473497-23 | DPGKEIQIDG ITTKVIDSYH LYNKDGIKLE ITVLKLDRNE KFRDIRKYIP |
| hrv-24 | DPGREVQIDG INTTVEDSYD LYNKDGIKLE ITVLKLKRNE KFKDIRKYLP |
| ef173416-24 | DPGREVQIDG INTTVEDSYD LYNKDGIKLE ITVLKLKRNE KFKDIRKYLP |
| hrv-25 | DPGNEIQIDG IATKVEDSYD LYNKDGVKLE ITVLKLKRNE KFKDIRKYIP |
| dq473508-28 | DAGREVEIDG IKTQIDDAYD LHNSQGVKLE ITVLKLHRNE KFRDIRKYIP |
| hrv-29 | DPGSEVQIDG ITTKVEDSFD LYNKDGVKLE ITVLKLKRNE KFKDIRKYIP |
| dq473512-30 | DPGKEIQIDG ITTKVTDSYD LYNKDGIKLE ITVLKLDRNE KFRDIRKYIP |
| hrv-30 | DPGKEIQIDG ITTKVTDSYD LYNKDGIKLE ITVLKLDRNE KFRDIRKYIP |
| hrv-31 | DPGNEIQIDG ITTKVEDSYD LFNKDGIKLE ITVLKLKRNE KFKDIRKYIP |
| hrv-32 | DPGKTVQING IITNVIDSYD LHNKDGVKLE ITVIKLDRNE KFRDIRKYIP |
| hrv-33 | DPGKEVQIDG ISTKVEDSYD LYNKDGIKLE ITVLKLQRNE KFRDIRKYIP |
| hrv-34 | DPGKEVQIDG ITTKVEDSFD LFNKDGVKLE ITVLKLKRNE KFKDIRKYIP |
| dq473501-34 | DPGKEVQIDG ITTKVEDSFD LFNKDGVKLE ITVLKLKRNE KFKDIRKYIP |
| dq473505-36 | DPGSEILVDG VKVKISDSYD LHNHEGVKLE ITVVKLVRNE KFKDIRKYLP |
| hrv-38 | DPGNEVQIDG ITTKVEDSYD LFNKDGVKLE ITVLKLKRNE KFKDIRKYIP |
| dq473495-38 | DPGNEVQIDG ITTKVEDSYD LFNKDGVKLE ITVLKLKRNE KFKDIRKYIP |
| ay751783-39 | DPDKEVQIDG ITTKVLDSYD LYNKDGVKLE ITVLKLDRNE KFRDIRKYIP |
| hrv-40 | DPGKEVQIDG ITTEVQDSYD LYNKDGIKLE ITVLKLKRNE KFKDIRKYIP |
| dq473491-41 | DPSSMIYVNG IATKVKDSYD LYNKQGIKLE ITVVILDRNE KFRDIRKYIP |
| hrv-43 | DPGKEIYING IKVKVSDSYD LYNKDGVKLE ITVVKLDRNE KFRDIRRYIP |
| dq473499-44 | DPGSEVQIDG IATKVEDSFD LYNKDGVKLE ITVLKLKRNE KFKDIRKYIP |
| hrv-45 | DPSRTVKVDG VETEVLDSYD LYNKEGVKLE ITVLILSRNE KFRDIRKYIP |
| dq473506-46 | DAGQEVEIDG IKTKVSDAYD LHNMHGVKLE ITVLKLSRNE KFRDIRKYIP |
| hrv-47 | DPGGEIQIDG ITTKVEDSYD LFNKDGIKLE ITVLKLKRNE KFKDIRKYIP |
| dq473496-49 | DPGKEIQVDG ITTKVADSYD LYNKDGIKLE ITVLKLDRNE KFRDIRKYIP |
| hrv-49-f04 | DPGKEIQVDG ITTKVADSFD LYNKDGIKLE ITVLKLDRNE KFRDIRKYIP |
| hrv-50 | DPGKEVQIDG ITTKVEDSYD LFNKDGVKLE ITVLKLKRNE KFKDIRKYIP |
| hrv-51 | DVGRTVDIDG IQTKVADAYD LHNSQGVKLE ITVVKLNRNE KFRDIRKYIP |
| dq473507-53 | DAGQEVEVDG IKTKVSDAYD LYNTQGVKLE ITVLKLNRNE KFRDIRKYIP |
| hrv-54 | DPGKEVQIDG ITTEVQDSYD LYNRDGIKLE ITVLKLKRNE KFKDIRKYIP |
| hrv-54-f05 | DPGKEVQIDG ITTEVQDSYD LYNKDGIKLE ITVLKLKRNE KFKDIRKYIP |
| dq473511-55 | DPGKEIQVDG ITTKVEDSYD LFNKDGVKLE ITVLKLKRNE KFKDIRKYIP |
| hrv-56 | DPGHEVQIDG ITTKVEESFD LFNKDGVKLE ITVLKLKRNE KFKDIRKYIP |
| hrv-57 | DPGKEVQIDG ITTGVEDSYD LFNKDGVKLE ITVLKLKRNE KFKDIRKYIP |
| hrv-58 | DPGSEILVDG VKVKISDSYD LHNHEGIKLE ITVVKLVRNE KFKDIRKYLP |
| dq473500-59 | DPGGEVQIDG IVTKVEDSYD LFNKDGIKLE ITVLKLKRNE KFRDIRKYIP |
| hrv-60 | DPGKEVQIDG ITTTVQDSYD LYNKDGVKLE ITVLKLKRNE KFKDIRKYIP |
| hrv-61 | DPGKEIYING IATRVSDSYD MYNKQGIKLE ITAVLLDRNE KFRDIRRYIP |
| hrv-62 | DPGNEIQIDG ITTKVEGSYD LYNKEGVKLE ITVLKLKRNE KFKDIRKYIP |
| hrv-63 | DPGHEVQIDG ITTKVEDSFD LFNKDGVKLE ITVLKLKRNE KFKDIRKYIP |
| hrv-64 | DPGKTIQING INTNVLDSYD LYNKNGVKLE ITVVKLDRNE KFRDIRKYIP |
| ef173417-64 | DPGKTIQING INTNVLDSYD LYNKNGVKLE ITVVKLDRNE KFRDIRKYIP |
| hrv-65 | DAGKTIDING IQTRVADAYD LYNSQGVKLE ITVVKLDRNE KFRDIRKYIP |
| hrv-66 | DPGKEVQIDG ITTKVEDSYD LYNKDGVKLE ITVLKLKRNE KFKDIRKYIP |
| hrv-67 | DPGKTVQING IITNVTDSYD LHNKEGIKLE ITVIKLDRNE KFRDIRKYIP |
| hrv-68 | DAGKEVEIDG IKTQIDDAYD LYNSQGIKLE ITVLKLHRNE KFRDIRKYIP |
| hrv-71 | EVGKSVEIDG IQVKVSDSYD LYNSHGVKLE ITVVKLNRNE KFRDIRKYIP |
| dq473492-73 | DPGKEVYING IATKVSDSYD LYNKQGVKLE ITAIVLDRNE KFRDIRKYIP |
| dq473494-74 | DPGTTIQIDG INTKVLDSYD LHNKDGIKLE ITVIKLDRNE KFRDIRKYIP |
| dq473510-75 | DPGKQVQING INVNVSDSYD LYNKDGVKLE ITVIKLDRNE KFRDIRKYIP |
| dq473502-76 | DPGKEVQIDG ISTKVEDSYD LYNKDGVKLE ITVLKLQRNE KFRDIRKYIP |
| hrv-76 | DPGKEVQIDG ISTKVEDSYD LYNKDGVKLE ITVLKLQRNE KFRDIRKYIP |
| hrv-77 | DPGREIQVDG ITTEVQDSYD LYNKDGVKLE ITVLKLKRNE KFKDIRKYIP |
| hrv-78 | EPGSQIEIDG VKTNVVDSYD LYNKDGVKLE ITVVKLNRNE KFRDIRKYLP |
| ef173418-78 | EPGSQIEIDG VKTNVVDSYD LYNKDGVKLE ITVVKLNRNE KFRDIRKYLP |
| hrv-80 | DVGGEIEVDG IKTKVLDSYD LYNKQGVKLE ITVIKLDRNE KFRDIRKYIP |
| hrv-81 | DPGSEIQVNG IHTKILDSYD LYNKEGVKLE ITVLKLDRNE KFRDIRKYIP |
| hrv-81-f06 | DPGSEIQVNG IQTKILDSYD LFNKEGVKLE ITVLKLDRNE KFRDIRKYIP |
| hrv-81-f07 | DPGSEIQVNG IQTKILDSYD LFNKEGVKLE ITVLKLDRNE KFRDIRKYIP |
| hrv-82 | DPGNTIQIDG INVKISDSYD LYNKNGVKLE ITVIKLDRNE KFRDIRKYIP |
| dq473509-82 | DPGKTIQIDG INVKISDSYD LYNKNGVKLE ITVVKLDRNE KFRDIRKYIP |
| hrv-85 | DPGKEVQIDG ITTQVQDSYD LYNKDGIKLE ITVLKLKRNE KFKDIRKYIP |
| pico-85 | DPGKEVQIDG ITTQVQDSYD LYNKDGIKLE ITVLKLKRNE KFKDIRKYIP |
| dq473504-88 | DPGSEVMIDG VKIKVLDSYD LHNHEGVKLE ITVIKLARNE KFKDIRKYLP |
| a10937-89 | DPGSEILVDG VKVKVSDSYD LHNHEGVKLE ITVVKLIRNE KFKDIRKYLP |
| hrv-89 | DPGSEILVDG VKVKVSDSYD LHNHEGVKLE ITVVKLIRNE KFKDIRKYLP |
| hrv-89-f08 | DPGSEILIDG VKVKVSDSYD LHNHEGVKLE ITVVKLARNE KFKDIRKYLP |
| hrv-89-f09 | DPGSEILIDG VKVKVSDSYD LHNHEGVKLE ITVVKLARNE KFKDIRKYLP |
| m16248-89 | DPGSEILVDG VKVKVSDSYD LHNHEGVKLE ITVVKLIRNE KFKDIRKYLP |
| hrv-90 | DPGKEIQIDG ISTKVEDSYD LYNKDGIKLE ITVLKLKRNE KFKDIRKYIP |
| hrv-94 | DPGKTIQING INTNVTDSYD LYNKNGIKLE ITVVKLDRNE KFRDIRKYIP |
| ef173419-94 | DPGKTIQING INTNVTDSYD LYNKNGIKLE ITVVKLDRNE KFRDIRKYIP |
| hrv-95 | DPGKSVNIDG VEVKVVDSYD LFNKEGVKLE ITVILLSRNE KFRDIRKYIP |
| hrv-96 | DPGKEISING IVTKVSDSYD MYNKQGIKLE ITAVVLDRNE KFRDIRKYIP |
| hrv-98 | DPGKEVQIDG ITTEVQDSYD LYNKDGIKLE ITVLKLKRNE KFKDIRKYIP |
| hrv-100 | DPGREVQIDG IATKVEDSYD LYNKDGIKLE ITVLKLKRNE KFKDIRKYIP |
| ef186077-qpm | QPEDTILIDG VETKILKQTI ITDENDVDTE VTILWLDRNE KFRDIRRFIP |
| ef077279-nat001 | QVEDTILIDG VETKVLKQTI VTDENDVDTE ITILWLDRNE KFRDIRRFIP |
| ny1078 | QADGTILIDG VETKILKQTI VTDESDVDTE ITILWLDRNE KFRDIRRFLP |
| ef582385-c024 | EPGDVVDIDG RLVRVLKQQV LTDMNDVDTE VTVLWLDQNE KFRDIRRFIP |
| ef077280-nat045 | ECDKEILIDG IPTKILKQQI ITDASDVDTE VTLLWLDRNE KFRDIRRFIP |
| ef582387-c026 | QPEDTILIDG VETKILKQTI ITDENDVDTE ITILWLDRNE KFRDIRRFIP |
| ef582386-c025 | ECGDTILIDN IQTKVIKQQI ITDSNDVDTE ITIVWLDRNE KFRDIRRFIP |
| dq473485-03 | QPGDNVLVNG QKIQIKDKYK LVDPDNTNLE LTIIELDRNE KFRDIRGFI. |
| ef173422-03 | QPGDNVLVNG QKIQIKDKYK LVDPDNTNLE LTIIELDRNE KFRDIRGFI. |
| dq473490-04 | QPSDQVLING FSTNVRDKWK LVDPDNLNLE ITVIELDRNE KFRDIRMFI. |
| hrv-05 | QPSDQVLING FATKIKDKWK LVDPDNLNLE ITVLELDRNE KFRDIRMFI. |
| dq473486-06 | QPDNKVLVNG QAVQIKDKYK LVDPDNINLE LTVLMLDRNE KFRDIRSFI. |
| l05355-14 | QPGDDVLVNG QKIRVKDKYK LVDPENINLE LTVLTLDRNE KFRDIRGFI. |
| k02121-14 | QPGDDVLVNG QKIRVKDKYK LVDPENINLE LTVLTLDRNE KFRDIRGFI. |
| ef173420-17 | QPDDEVLVNG QPVKLKDKYK LLDPENTNLE LTILILDRNE KFRDIRAFI. |
| hrv-26 | QPGEQVLING FSTKLKDKWK LVDPDDINLE ITVLELDRNE KFRDIRMFI. |
| hrv-27 | QPENEVLLNG YNVKIKDKWR LVDPDNLNLE ITVLELDRNE KFRDIRMFI. |
| ef173421-27 | QPENEVLLNG YNVKIKDKWR LVDPDNLNLE ITVLELDRNE KFRDIRMFI. |
| dq473487-35 | QPGDSVLVNG QKIQVRDKYK LVDPENVNLE LTILILDRNE KFRDIRNFI. |
| hrv-35 | QPGDSVLVNG QKIQVRDKYK LVDPENVNLE LTILILDRNE KFRDIRNFI. |
| ef173423-37 | QPGNSVLLNG QKIQIKDKYK LMDPDNINLE LTVLILDRNE KFRDIRNFI. |
| hrv-42 | QPSEKVLING FATNVKDKWK LVDPDNLNLE ITVLELDRNE KFRDIRMFI. |
| dq473488-48 | QPSDEVLING QTVKLKDKYK LLDPENTNLE LTILVLDRNE KFRDIRAFI. |
| hrv-52 | QPDDEVLVNG QPVKLKDKYK LMDPENTNLE LTIIVLDRNE KFRDIRAFI. |
| hrv-52-f10 | QPDDEVLVNG QPVKLKDKYK LMDPENTNLE LTIIVLDRNE KFRDIRAFI. |
| ef173424-52 | QPDDEVLVNG QPVKLKDKYK LMDPENTNLE LTIIVLDRNE KFRDIRAFI. |
| hrv-69 | QPGDEVLVNG QPIKLKDKYK LLDPENTNLE LTIVVLDRNE KFRDIRAFI. |
| dq473489-70 | QPDDEVLVNG QPVKLKDKYK LLDPENTNLE LTILVLDRNE KFRDIRAFI. |
| hrv-72 | QPNDSVLING QKVQIKDKFK LVDPDNINLE LTILDLNRNE KFRDIRAFI. |
| hrv-79 | QPSDSVLVNG QKIQIKDKYK LVDPENVNLE LTILVLDRNE KFRDIRGFI. |
| hrv-83 | QPTDNVLLNG QKVQIKDKYK LVDPENVNLE LTIITLDRNE KFRDIRGFI. |
| hrv-84 | QPEDEVLVNG FKTRVSDKWK LVDPDNMNLE ITVLQLDRNE KFRDIRSFI. |
| hrv-86 | HPGQEVLLNG QKIKVSDKYR LIDSDSINLE LTVLTLDRNE KFRDIRPFI. |
| hrv-91 | QPDDEVLVNG QPVKLKDKYK LLDSENTNLE LTILVLDRNE KFRDIRAFI. |
| hrv-92 | QPSESVLLNG QKVHLKDKYK LVDPENVNLE LTIIVLDRNE KFRDIRGFI. |
| ef173425-93 | QPENEVLLNG YNTKIKDKWR LVDPDNLNLE ITVLELDRNE KFRDIRMFI. |
| hrv-97 | QPENEVLLNG YNIKIKDKWK LIDPDNLNLE ITVLELDRNE KFRDIRMFI. |
| hrv-99 | QPTEEVLING FTVKIKDKWK LVDPDNLNLE ITVLELDRNE KFRDIRMFI. |
| | 1701 1750 |
| annotation | .......BBB BB......BB BBBBBBBBBB BBBBBB...B BBBBBBBB.. |
| af499637-cva13 | TQITETNDGV LIVNTSKYPN MYVPVGAVTE QGYLNLGGRQ TARTLMYNFP |
| af546702-cva21 | TQITETNDGV LIVNTSKYPN MYVPVGAVTE QGYLNLSGRQ TARTLMYNFP |
| v01149-pv1m | TQITETNDGV LIVNTSKYPN MYVPVGAVTE QGYLNLGGRQ TARTLMYNFP |
| d00239-01 | ETEDDYPECN LALSANQVEP TIIKVGDVVS YGNILLSGNQ TARMLKYNYP |
| pico-01 | ETEDDYPECN LALSANQDEP TIIKVGDVVS YGNILLSGNQ TARMLKYNYP |
| hrv-01 | ETEDDYPECN LALSANQDEP TIIKVGDVVS YGNILLSGNQ TARMLKYNYP |
| x02316-02 | NNEDDYPNCN LALLANQPEP TIINVGDVVS YGNILLSGNQ TARMLKYSYP |
| hrv-07 | TRDDDYLGCN LALLANQDEP TILNVGDAIS YGNILLSGTN TARMIKYHYP |
| dq473503-07 | TRDDDYLGCN LALLANQDEP TILNVGDAIS YGNILLSGTN TARMIKYHYP |
| hrv-08 | NSEDDYTNCN LALVANQEVP QILEVGDVVS YGNILLSGNN TARMLKYDYP |
| pico-09 | EREDDYPECN LALVANQIEP TIISVGDVIS YGNILLSGNQ TARMLKYHYP |
| hrv-09 | EREDDYPECN LALVANQVEP TIISVGDVIS YGNILLSGNQ TARMLKYHYP |
| hrv-09-f01 | EREDDYPECN LALVANQDEP TIISVGDVIS YGNILLSGNQ TARMLKYHYP |
| hrv-09-f02 | EREDDYPECN LALVANQDEP TIISVGDVIS YGNILLSGNQ TARMLKYHYP |
| dq473498-10 | ENEDDYPDCN LALSANQPET TIINVGDVVS YGNILLSGTQ TARMLKYNYP |
| hrv-10 | ENEDDYPDCN LALSANQPET TIINVGDVVS YGNILLSGTQ TARMLKYNYP |
| ef173414-11 | EREDDYPECN LALSANQPET TIINVGDVVS YGNILLSGNQ TARMLKYNYP |
| ef173415-12 | DREDDYPECH LALTANQEQP TILEVGDAVS YGNILLSGNE TARMIKYNYP |
| hrv-13 | EREDDYPDCN LALVANQPEP TIINVGDVIS YGNILLSGNQ TARMLKYNYP |
| hrv-13-f03 | EREDDYPDCN LALVANQPEP TIINVGDVIS YGNILLSGNQ TARMLKYNYP |
| dq473493-15 | ESEDDYPDCN LALVANQVEP TIISVGDVIS YGNILLSGSQ TARMLKYNYP |
| l24917-16 | ESEDDYPECN LALVANQTEP TIIKVGDVVS YGNILLSGTQ TARMLKYNYP |
| hrv-18 | EDEDDYPDCN LALSANQPET TIINVGDVVS YGNILLSGNQ TARMLKYNYP |
| hrv-19 | EKEDDYPDCN LALVANQPEP TILSVGDVVS YGNILLSGNQ TARMLKYNYP |
| hrv-20 | ETEDDYPECH LALVANQQEP TILEVGDCCS YGNILLSGNQ TARMIKYNYP |
| hrv-21 | ENEDDYPDCN LALSANQPET TIINVGDVIS YGNILLSGTQ TARMLKYNYP |
| hrv-22 | EREDDYPDCN LALVANQPEP TIINVGDVVS YGNILLSGDQ TARMLKYNYP |
| dq473497-23 | NNEDDYPDCN LALLANQPEP TIINVGDVVS YGNILLSGSQ TARMLKYNYP |
| hrv-24 | ENEDDYPDCS LALSANQPET TIINVGDVIS YGNILLSGNQ TARMLKYNYP |
| ef173416-24 | ENEDDYPDCS LALSANQPET TIINVGDVIS YGNILLSGNQ TARMLKYNYP |
| hrv-25 | ENEDDYPDCN LALSANQPET TIINVGDVVS YGNILLSGNQ TARMLKYNYP |
| dq473508-28 | ESEDDYPECH LALVANQQEP TILEVGDCCS YGNILLSGNQ TARMIKYNYP |
| hrv-29 | ENEDDYPDCN LALSANQPET TIINVGDVVS YGNILLSGNQ TARMLKYNYP |
| dq473512-30 | SNEDDYPDCN LALLANQPEP TIINVGDVVS YGNILLSGNQ TARMLKYNYP |
| hrv-30 | SNEDDYPDCN LALLANQPEP TIINVGDVVS YGNILLSGNQ TARMLKYNYP |
| hrv-31 | ENEDDYPDCN LALSANQPET TIINVGDVVS YGNILLSGNX TARMLKYNYP |
| hrv-32 | EREDDYPDCN LALVANQPEP TIINVGDVIS YGNILLSGNQ TARMLKYHYP |
| hrv-33 | EKEDDYPECN LALSANQPET TIISVGDVVS YGNILLSGNQ TARMLKYNYP |
| hrv-34 | EHEDDYPDCN LALSANQPET TIIKVGDVVS YGNILLSGNQ TARMLKYNYP |
| dq473501-34 | EHEDDYPDCN LALSANQPET TIIKVGDVVS YGNILLSGNQ TARMLKYNYP |
| dq473505-36 | SREDDYPACN LALLANQDEP TIINVGDAES YGNILLSGTN TARMIKYHYP |
| hrv-38 | ENEDDYPDCN LALSANQPET TIINVGDVIS YGNILLSGNQ TARMLKYNYP |
| dq473495-38 | ENEDDYPDCN LALSANQPET TIINVGDVIS YGNILLSGNQ TARMLKYNYP |
| ay751783-39 | ENEDDYPECN LALSANQPET TILNVGDVVS YGNILLSGNQ TARMLKYNYP |
| hrv-40 | ENEDDYPDCN LALSANQPET TIINVGDVVS YGNILLSGSQ TARMLKYNYP |
| dq473491-41 | EKEDDYPECN LALVANQPEP TIINVGDVIS YGNILLSGNQ TARMLKYHYP |
| hrv-43 | EKEDDYPECN LALVANQPEP TIINVGDVVS YGNILLSGNQ TARMLKYNYP |
| dq473499-44 | ENEDDYPDCN LALSANQPET TIINVGDVVS YGNILLSGNQ TARMLKYNYP |
| hrv-45 | NSEDDYVNCN LALVANQDMP QILEVGDVVS YGNILLSGNN TARMLKYEYP |
| dq473506-46 | ESEDDYPECC LALVANQTEP TILEVGDCCS YGNILLSGNQ TARMIKYNYP |
| hrv-47 | ENEDDYPDCN LALSANQPET TIINVGDVVS YGNILLSGNQ TARMLKYNYP |
| dq473496-49 | NNEDDYPDCN LALLANQPEP TIINVGDVIS YGNILLSGNQ TARMLKYSYP |
| hrv-49-f04 | NNEDDYPDCN LALLANQPEP TIINVGDVVS YGNILLSGNQ TARMLKYNYP |
| hrv-50 | ENEDDYPDCN LALSANQPET TIIRVGDVVS YGNILLSGNQ TARMLKYNYP |
| hrv-51 | DSEDDYPDCC LALLANQPEP TIIEIGDSCS YGNILLSGNQ TARMIKYNYP |
| dq473507-53 | ESEDDYPECC LALVANQVEP TILEVGDCCS YGNILLSGNQ TARMIKYNYP |
| hrv-54 | ENEDDYPDCN LALSANQPET TIINVGDVVS YGNILLSGNQ TARMLKYNYP |
| hrv-54-f05 | ENEDDYPDCN LALSANQPET TIINVGDVVS YGNILLSGNQ TARMLKYNYP |
| dq473511-55 | ESESDYPDCN LALSANQPET TILNVGDVIS YGNILLSGTQ TARMLKYNYP |
| hrv-56 | ENEDDYPDCN LALSANQPET TIINVGDVVS YGNILLSGTQ TARMLKYNYP |
| hrv-57 | EREDDYPDCN LALSANQPET TIINVGDVVS YGNILLSGNQ TARMLKYNYP |
| hrv-58 | SREDDYPACN LALLANQDEP TIINVGDAES YGNILLSGTN TARMIKYHYP |
| dq473500-59 | ESEDDYPDCS LALSANQPET TIINVGDVVS YGNILLSGNQ TARMLKYNYP |
| hrv-60 | ENEDDYPDCN LALSANQPET TIISVGDVVS YGNILLSGNQ TARMLKYNYP |
| hrv-61 | EREDDYPECN LALVANQPEP TIISVGDVIS YGNILLSGNQ TARMLKYNYP |
| hrv-62 | ESEDDYPDCN LALSANQPET TIINVGDVVS YGNILLSGNQ TARMLKYNYP |
| hrv-63 | ESEDDYPDCN LALSANQPET TIINVGDVVS YGNILLSGTQ TARMLKYNYP |
| hrv-64 | EREDDYPDCN LALVANQPEP TIINVGDVVS YGNILLSGNQ TARMLKYNYP |
| ef173417-64 | EREDDYPDCN LALVANQPEP TIINVGDVVS YGNILLSGNQ TARMLKYNYP |
| hrv-65 | DTEDDYPECC LALLANQPEP TIIEVGDSCS YGNILLSGNQ TARMIKYNYP |
| hrv-66 | ENEDDYPDCN LALSANQPET TIINVGDVVS YGNILLSGTQ TARMLKYSYP |
| hrv-67 | EREDDYPDCN LALVANQVEP TIISVGDVVS YGNILLSGNQ TARMLKYHYP |
| hrv-68 | ESEDDYPECH LALVANQQEP TILEVGDCCS YGNILLSGNQ TARMIKYNYP |
| hrv-71 | DSEDDYSDCC LALLANQPEP TIIEVGDSCS YGNILLSGTQ TARMIKYNYP |
| dq473492-73 | EREDDYPDCN LALVANQSEP TIINVGDVIS YGNILLSGNQ TARMLKYNYP |
| dq473494-74 | EREDDYPECN LALVANQIEP TIISVGDVVS YGNILLSGNQ TARMLKYNYP |
| dq473510-75 | EKEDDYPECN LALVANQPEP TIIGVGDVVS YGNILLSGNQ TARMLKYNYP |
| dq473502-76 | EKEDDYPECN LALSANQPET TIISVGDVVS YGNILLSGNQ TARMLKYNYP |
| hrv-76 | EKEDDYPECN LALSANQPET TIISVGDVVS YGNILLSGNQ TARMLKYNYP |
| hrv-77 | ENEDDYPDCN LALSANQPET TIINVGDVVS YGNILLSGNQ TARMLKYNYP |
| hrv-78 | EHEDDYMECN LALVANQEQP TIIEVGDVCS YGNILLSGTQ TARMLKYNYP |
| ef173418-78 | EHEDDYMECN LALVANQEQP TIIEVGDVCS YGNILLSGTQ TARMLKYNYP |
| hrv-80 | EVEDDYVDCC LALTANQSEP TILQVGDSVS YGNILLSGNQ TARMIKYNYP |
| hrv-81 | ETEDDYPECN LALVANQPEP TIIKVGDVIS YGNILLSGTQ TARMLKYNYP |
| hrv-81-f06 | ETEDDYPECN LALVANQPEP TIIKVGDVIS YGNILLSGTQ TARMLKYNYP |
| hrv-81-f07 | ETEDDYPECN LALVANQPEP TIIKVGDVVS YGNILLSGTQ TARMLKYNYP |
| hrv-82 | EKEDDYPDCN LALVANQPEP TILNVGDVVS YGNILLSGNQ TARMLKYNYP |
| dq473509-82 | EKEDDYPECN LALVANQPEP TILNVGDVVS YGNILLSGNQ TARMLKYNYP |
| hrv-85 | ENEDDYPDCN LALSANQPET TIINVGDVVS YGNILLSGNQ TARMLKYNYP |
| pico-85 | ENEDDYPDCN LALSANQPET TIINVGDVVS YGNIILSGNQ TARMLKYNYP |
| dq473504-88 | TREDDYPACN LALLANQDEP TILNVGDAIS YGNILLSGTN TARMIKYHYP |
| a10937-89 | SREDDYPACN LALLANQDEP TIISVGDAVS YGNILLSGTN TARMIKYHYP |
| hrv-89 | SREDDYPACN LALLANQDEP TIISVGDAVS YGNILLSGTN TARMIKYHYP |
| hrv-89-f08 | SREDDYPACN LALLANQDEP TIISVGDAVS YGNILLSGTN TARMIKYHYP |
| hrv-89-f09 | SREDDYPACN LALLANQDEP TIISVGDAES YGNILLSGTN TARMIKYHYP |
| m16248-89 | SREDDYPACN LALLANQDEP TIISVGDAVS YGNILLSGTN TARMIKYHYP |
| hrv-90 | ESEDDYPDCN LALSANQPET TIINVGDVVS YGNILLSGNQ TARMLKYNYP |
| hrv-94 | EREDDYPDCN LALVANQPEP TIINVGDVVS YGNILLSGDQ TARMLKYNYP |
| ef173419-94 | EREDDYPDCN LALVANQPEP TIINVGDVVS YGNILLSGDQ TARMLKYNYP |
| hrv-95 | NSEDDYTNCN LALVANQEVP QILEVGDVVS YGNILLSGNN TARMLKYDYP |
| hrv-96 | EGEDDYPECN LALVANQPEP TIINVGDVIS YGNILLSGTQ TARMLKYNYP |
| hrv-98 | ENEDDYPDCN LALSANQPET TIINVGDVVS YGNILLSGNQ TARMLKYNYP |
| hrv-100 | ENEDDYPDCN LALSANQPET TIINVGDVVS YGNILLSGNQ TARMLKYNYP |
| ef186077-qpm | DYQRDWQNMR LVTNVPKFPM LDIEIGDVIP YGDINLSGNP TTRLLKYDYP |
| ef077279-nat001 | DYQRDWQGMR LVTNVPKFPM FDAEVGDVIP YGDINLSGNP TTRLLKYDYP |
| ny1078 | DYQRDWQNMR LVTNVPKFPM LDIEVGDVVP YGDINLSGNP TTRLLKYDYP |
| ef582385-c024 | EHQQDWHNIH LATNVTKFPM LNVEVGHTVP YGEINLSGNA TCRLYKYDYP |
| ef077280-nat045 | ETIEEWHHIR LVTNVSKFPM FFADVGTATP YGEITLSGNP TCRLLKYDYP |
| ef582387-c026 | DYQRDWQNMR LVTNVPKFPM LDIEIGDVIP YGDINLSGNP TTRLLKYDYP |
| ef582386-c025 | EHIEEWRNMR LATNVPKFPM MDVEVGDVTP YGEINLSGNL TCRLLKYNYP |
| dq473485-03 | SEDLEGLDAT LVVHSNGFTN TILDVGPITM AGLINLSNTP TTRMIRYDYP |
| ef173422-03 | SEDLEGLDAT LVVHSNGFTN TILDVGPITM AGLINLSNTP TTRMIRYDYP |
| dq473490-04 | ADDPECNEAT LVINSNNFKD TILTVGTTTL AGIINLSNTP TARMIRYEFP |
| hrv-05 | ADDPECNEAS LVIHSNNFKD TILSVGTTTL AGIINLSNTP TARMIRYEFP |
| dq473486-06 | SEDMEGMEAT LVVHSNNFTN TILDVGPITM AGLINLSSTP TSRMIRYDYP |
| l05355-14 | SEDLEGVDAT LVVHSNNFTN TILEVGPVTM AGLINLSSTP TNRMIRYDYA |
| k02121-14 | SEDLEGVDAT LVVHSNNFTN TILEVGPVTM AGLINLSSTP TNRMIRYDYA |
| ef173420-17 | ADDPEGMEGT LVINSNNFKD TILEVGQLTF AGLINLSSVP TNRMIRYEYP |
| hrv-26 | ADEPECSDAT LVINSNNFKD TILSVGSTTL AGIINLSNTP TARMIRYEFP |
| hrv-27 | ADESECPDAS LVIHSNNYKD TIMSVGHITM AGIINLSNTP TARMIRYDYP |
| ef173421-27 | ADESECPDAS LVIHSNNYKD TIMSVGHITM AGIINLSNTP TARMIRYDYP |
| dq473487-35 | SEDVEGLEAT LVIHSNHFTN TILDVGPITM AGLINLSSTP TNRMIRYDYP |
| hrv-35 | SEDVEGLEAT LVIHSNHFTN TILDVGPITM AGLINLSSTP TNRMIRYDYP |
| ef173423-37 | SEDIEGMEAT LVVHSNNFTN TILEVGPITM AGLINLSSTP TSRMIRYDYP |
| hrv-42 | ADDPECNEAS LVIHSNNFKD TILTVGATTL AGIINLSNTP TARMIRYDFP |
| dq473488-48 | AEDPEGAEGT LVINSNNFKD TILEVGQLTF AGLINLSSVP TNRMIRYEYP |
| hrv-52 | AEDPEGMEGT LVINSNNFKD TILEVGQLTF AGLINLSSVP TNRMIRYEYP |
| hrv-52-f10 | AEDPEGMEGT LVINSNNFKD TILEVGQLTF AGLINLSSVP TNRMIRYEYP |
| ef173424-52 | AEDPEGMEGT LVINSNNFKD TILEVGQLTF AGLINLSSVP TNRMIRYEYP |
| hrv-69 | AEDPEGVEGT LVINSNNFRD TILEVGQLTF AGLINLSSVP TNRMIRYEYP |
| dq473489-70 | ADDPEGMEGT LVINSNNFKD TILEVGQLTF AGLINLSSVP TNRMIRYEYP |
| hrv-72 | SEDFEGVEAT LVVHSNNYTN TILEVGPVTM AGLINLSSTP TNRMIRYEYA |
| hrv-79 | SEDLEGLDAT LVIHSNNFTN TILDVGPITM AGLINLSSTP TNRMIRYDYP |
| hrv-83 | SEDLEGFDAS LVIHSNNFTN TILDVGPITM AGLINLSSTP TNRMIRYDYP |
| hrv-84 | AEDPECSEAT LVINSNNFKD TIITVGQATL VGMINLSNTP TTRMIRYDYP |
| hrv-86 | CEDPEGMEAT LVIHSNNFTN TILEVGQITM AGLINLSSTP TGRMIRYEYP |
| hrv-91 | ADDPEGMEGT LVINSNNFKD TILEVGQLTF AGLINLSSVP TNRMIRYEYP |
| hrv-92 | SEDAEGLDAS LVIHSNNYTN TILDVGPITM AGLINLSSTP TNRMIRYDYP |
| ef173425-93 | ADEVECPDAS LVIHSNNYKN TIMSVGHTTM AGIINLSNTP TARMIRYDYP |
| hrv-97 | ADDLECSEAS LVIHSNNYKN TIMSVGHITM AGIINLSNTP TARMIRYDYP |
| hrv-99 | ADEPECNEAS LVIHSNNFKD TILSVGSTTL AGIINLSSTP TARMIRYDFP |
| | 1751 1800 |
| annotation | .....C..BB BB..BBBBBB BBBB..BBBB BB.HHHHHH. .QGBBBBBBB |
| af499637-cva13 | TRAGQCGGVI TCTGKVIGMH VGGNGSHGFA AALKRSYFTQ SQGEIQWMKP |
| af546702-cva21 | TRAGQCGGII TCTGKVIGMH VGGNGSHGFA AALKRSYFTQ NQGEIQWMRS |
| v01149-pv1m | TRAGQCGGVI TCTGKVIGMH VGGNGSHGFA AALKRSYFTQ SQGEIQWMRP |
| d00239-01 | TKSGYCGGVL YKIGQILGIH VGGNGRDGFS AMLLRSYFTD TQGQIKISKH |
| pico-01 | TKSGYCGGVL YKIGQILGIH VGGNGRDGFS AMLLRSYFTD TQGQIKVNKH |
| hrv-01 | TKSGYCGGVL YKIGQILGIH VGGNGRDGFS AMLLRSYFTD TQGQIKVNKH |
| x02316-02 | TKSGYCGGVL YKIGQVLGIH VGGNGRDGFS AMLLRSYFTD VQGQITLSKK |
| hrv-07 | TKAGFCGGVL YKVGSILGIH VGGNGRDGFS AMLLQSYFSE TQGLITKELP |
| dq473503-07 | TKAGFCGGVL YKVGSILGIH VGGNGRDGFS AMLLQSYFSE TQGLITKELP |
| hrv-08 | TKSGFCGGVL YKVGQVIGIH VGGNGRQGFS AMLLKRYFNQ QQGQIIIKKP |
| pico-09 | TKSGYCGGVL YKIGSIIGIH VGGNGRDGFS AMLLRSYFNE TQGQIINSRN |
| hrv-09 | TKSGYCGGVL YKIGSIIGIH VGGNGRDGFS AMLLRSYFNE TQGQIISSRN |
| hrv-09-f01 | TKSGYCGGVL YKIGSIIGIH VGGNGRDGFS AMLLRSYFNE TQGQIVSSRN |
| hrv-09-f02 | TKSGYCGGVL YKIGSIIGIH VGGNGRDGFS AMLLRSYFNE TQGQIVSSRN |
| dq473498-10 | TKSGYCGGIL YKIGQVLGIH VGGNGRDGFA AMLLRSYFSE TQGEIIISKS |
| hrv-10 | TKSGYCGGIL YKIGQVLGIH VGGNGRDGFA AMLLRSYFSE TQGEIIISKS |
| ef173414-11 | TKSGYCGGIL YKIGQILGIH VGGNGRDGFA AMLLRSYFSE AQGQITTSKS |
| ef173415-12 | TKSGYCGGVL YKIGQIVGIH VGGNGRDGFS AMLLRSYFTN FQGVITMQDD |
| hrv-13 | TKSGYCGGVL YKIGQIIGIH VGGNGRDGFS AMLLRSYFNQ TQGEITVTKK |
| hrv-13-f03 | TKSGYCGGVL YKIGQIIGIH VGGNGRDGFS AMLLRSYFNQ TQGEITVAKK |
| dq473493-15 | TKSGYCGGVL YKIGSVIGIH VGGNGRDGFS AMLLRSYFSE TQGQITLKKK |
| l24917-16 | TKSGYCGGVL YKIGQILGIH VGGNGRDGFS SMLLRSYFTE QQGQIQISKH |
| hrv-18 | TKSGYCGGIL YKIGQVLGIH VGGNGRDGFA AMLLRSYFSE TQGQIVSSKS |
| hrv-19 | TKSGYCGGVL YKIGVVLGIH VGGNGRDGFS SMLLRSYFSE TQGEITKTEN |
| hrv-20 | TKSGFCGGVL YKIGLVLGIH VGGNGRDGFS AMLLRSYFTE QQGKITSKTS |
| hrv-21 | TKSGYCGGIL YKIGQVLGIH VGGNGRDGFA AMLLRSYFSE TQGQIINSKP |
| hrv-22 | TKSGYCGGVL YKIGNILGIH VGGNGKDGFS ATLLRSYFSE TQGQITKTTS |
| dq473497-23 | TKSGYCGGVL YKIGQVLGIH VGGNGRDGFS AMLLRSYFAD TQGQITLSKR |
| hrv-24 | TKSGYCGGIL YKIGQVLGIH VGGNGRDGFA AMLLRSYFSE TQGQIVTRKS |
| ef173416-24 | TKSGYCGGIL YKIGQVLGIH VGGNGRDGFA AMLLRSYFSE TQGQIVTRKS |
| hrv-25 | TKSGYCGGIL YKIGQVLGIH VGGNGRDGFA AMLLRSYFSE TQGQIITSKS |
| dq473508-28 | TKSGFCGGVL YKIGLVLGIH VGGNGRDGFS AMLLRSYFTE HQGKIVSKAN |
| hrv-29 | TKSGYCGGIL YKIGQVLGIH VGGNGKDGFA AMLLRSYFSE TQGQIITSKS |
| dq473512-30 | TKSGYCGGVL YKIGQVLGIH VGGNGRDGFS AMLLRSYFAD TQGQITLSKR |
| hrv-30 | TKSGYCGGVL YKIGQVLGIH VGGNGRDGFS AMLLRSYFAD TQGQITLSKR |
| hrv-31 | TKSGYCGGIL YKIGQVLGIH VGGNGRDGFA AMLLRSYFSE TQGQIVVTKP |
| hrv-32 | TKSGYCGGVL YKIGSIIGIH VGGNGRDGFS AMLLRSYFSE TQGQITSSRN |
| hrv-33 | TKSGYCGGIL YKIGQVLGIH VGGNGRDGFA AMLLRSYFSE TQGQITISKP |
| hrv-34 | TKSGYCGGIL YKIGQVLGIH VGGNGRDGFA AMLLRSYFTE AQGQIITSKS |
| dq473501-34 | TKSGYCGGIL YKIGQVLGIH VGGNGRDGFA AMLLRSYFTE AQGQIITSKS |
| dq473505-36 | TKAGYCGGVL YKVGSILGIH VGGNGRDGFS AMLLKSYFGE TQGLITKELP |
| hrv-38 | TKSGYCGGIL YKIGQVLGIH VGGNGRDGFA AMLLRSYFTE VQGQITTTKS |
| dq473495-38 | TKSGYCGGIL YKIGQVLGIH VGGNGRDGFA AMLLRSYFTE VQGQITTTKS |
| ay751783-39 | TKSGYCGGIL YKIGQVLGIH VGGNGRDGFS AMLLRSYFTD TQGQITLSKK |
| hrv-40 | TKSGYCGGIL YKIGQVLGIH VGGNGRDGFA AMLLRSYFTE TQGQIITSKS |
| dq473491-41 | TKSGYCGGVL YKIGQIMGIH VGGNGRDGFS AMLLRSYFSE TQGEITTTRK |
| hrv-43 | TKSGYCGGVL YKIGNVLGIH VGGNGRDGFS AMLLRSYFNE TQGKIITEKS |
| dq473499-44 | TKSGYCGGIL YKIGQVLGIH VGGNGKDGFA AMLLRSYFSE TQGQIITSKS |
| hrv-45 | TKSGFCGGVL YKVGQVIGIH VGGNGRQGFS AMLLKRYFNQ QQGQIILKKP |
| dq473506-46 | TKSGFCGGVL YKIGLVLGIH VGGNGRDGFS AMLLRSYFNE QQGKIVSRAD |
| hrv-47 | TKSGYCGGIL YKIGQVLGIH VGGNGRDGFA AMLLRSYFSE TQGQITTTKP |
| dq473496-49 | TKSGYCGGVL YKIGQVLGIH VGGNGRDGFS AMLLRSYFTD VQGQIISSKK |
| hrv-49-f04 | TKSGYCGGVL YKIGQVLGIH VGGNGRDGFS AMLLRSYFTD VQGQIVSSKK |
| hrv-50 | TKSGYCGGIL YKIGQVLGIH VGGNGRDGFA AMLLRSYFSE TQGQIITSKS |
| hrv-51 | TKSGFCGGIL YKIGLILGIH VGGNGRNGFS AMLLRSYFNE QQGQIMTKAD |
| dq473507-53 | TKSGFCGGVL YKIGLILGIH VGGNGRDGFS AMLLRSYFNE QQGKIVSKAE |
| hrv-54 | TKSGYCGGIL YKIGQVLGIH VGGNGKDGFA AMLLRSYFTE AQGQIVTTKP |
| hrv-54-f05 | TKSGYCGGIL YKIGQVLGIH VGGNGRDGFA AMLLRSYFTE AQGQIVTTKS |
| dq473511-55 | TKSGYCGGIL YKIGQVLGIH VGGNGRDGFA AMLLRSYFSE TQGQITNSRP |
| hrv-56 | TKSGYCGGIL YKIGQVLGIH VGGNGRDGFA AMLLRSYFSE TQGKIVTSKS |
| hrv-57 | TKSGYCGGIL YKIGQVLGIH VGGNGRDGFA AMLLRSYFSE AQGQIISSKS |
| hrv-58 | TKAGYCGGVL YKVGSILGIH VGGNGRDGFS AMLLKSYFGE TQGLITKELP |
| dq473500-59 | TKSGYCGGIL YKIGQVLGIH VGGNGRDGFS AMLLRSYFSE TQGQIISPKP |
| hrv-60 | TKSGYCGGIL YKIGQVLGIH VGGNGRDGFA AMLLRSYFTE AQGQITTTKT |
| hrv-61 | TKSGYCGGVL YKIGQIIGIH VGGNGRDGFS AMLLRSYFSE TQGEIITSKK |
| hrv-62 | TKSGYCGGIL YKIGQVLGIH VGGNGRDGFA AMLLRSYFSE TQGQIITSRS |
| hrv-63 | TKSGYCGGIL YKIGQVLGIH VGGNGRDGFA AMLLRSYFSE TQGKVIVSKF |
| hrv-64 | TKSGYCGGVL YKIGSVLGIH VGGNGRDGFS ATLIRSYFSE AQGQITKRTN |
| ef173417-64 | TKSGYCGGVL YKIGSVLGIH VGGNGRDGFS ATLIRSYFSE AQGQITKRTN |
| hrv-65 | TKSGFCGGVL YKIGLVLGIH VGGNGRNGFS AMLLRSYFNE QQGQIMSRAD |
| hrv-66 | TKSGYCGGIL YKIGQIIGIH VGGNGRDGFS AMLLRSYFSE TQGQIVAKKS |
| hrv-67 | TKSGYCGGVL YKIGSVIGIH VGGNGRDGFS AMLLRSYFSE TQGQITSSRN |
| hrv-68 | TKSGFCGGVL YKIGLVLGIH VGGNGRDGFS AMLLRSYFTE QQGKIVSKVS |
| hrv-71 | TKSGFCGGVL YKIGLILGIH VGGNGRNGFS AMLLRSYFNE QQGKIVSTSD |
| dq473492-73 | TKSGYCGGVL YKIGQIIGIH VGGNGRDGFS AMLLRSYFNQ TQGEITVTKK |
| dq473494-74 | TKSGYCGGVL YKIGSVLGIH VGGNGRDGFS AMLLRSYFSE TQGQLTTTKK |
| dq473510-75 | TKSGYCGGVL YKIGNILGIH VGGNGRDGFS AMLLRSYFNE TQGKIVAAKD |
| dq473502-76 | TKSGYCGGIL YKIGQVLGIH VGGNGRDGFA AMLLRSYFSE TQGQITISKP |
| hrv-76 | TKSGYCGGIL YKIGQVLGIH VGGNGRDGFA AMLLRSYFSE TQGQITISKP |
| hrv-77 | TKSGYCGGIL YKIGQVLGIH VGGNGRDGFA AMLLRSYFTE AQGQIVTTKP |
| hrv-78 | TKSGFCGGVL YKVGHIIGIH VGGNGRDGFS AMILRSYFNE NQGLIKNKTD |
| ef173418-78 | TKSGFCGGVL YKIGHIIGIH VGGNGRDGFS AMILRSYFNE NQGLIKNKTD |
| hrv-80 | TKSGYCGGVL YKIGLIIGIH VGGNGKDGFS AMLLRSYFNN HQGQIVNKTT |
| hrv-81 | TKSGYCGGVL YKIGLILGIH VGGNGRDGFS SMLLRSYFTE QQGQIKISQH |
| hrv-81-f06 | TKSGYCGGVL YKIGLVLGIH VGGNGRDGFS SMLLRSYFTE QQGQIKISKH |
| hrv-81-f07 | TKSGYCGGVL YKIGLVLGIH VGGNGRDGFS SMLLRSYFTE QQGQIKISKH |
| hrv-82 | TKSGYCGGVL YKIGNILGIH VGGNGKDGFS ATLLRSYFSE TQGQITKTAN |
| dq473509-82 | TKSGYCGGVL YKIGNVLGIH VGGNGKDGFS ATLLRSYFSE TQGQITKTAN |
| hrv-85 | TKSGYCGGIL YKIGQVLGIH VGGNGRDGFA AMLLRSYFSE TQGQIISTKS |
| pico-85 | TKSGYCGGIL YKIGQVLGIH VGGNGRDGFA AMLLRSYFSE TQGQIISTKS |
| dq473504-88 | TKAGYCGGVL YKVGSILGIH VGGNGRDGFS AMLLKSYFNE TQGLITKELP |
| a10937-89 | TKAGYCGGVL YKVGSILGIH VGGNGRDGFS AMLLKSYFGE TQGLITKELP |
| hrv-89 | TKAGYCGGVL YKVGSILGIH VGGNGRDGFS AMLLKSYFGE TQGLITKELP |
| hrv-89-f08 | TKAGYCGGVL YKVGSILGIH VGGNGRDGFS AMLLKSYFGE TQGLITKELP |
| hrv-89-f09 | TKAGYCGGVL YKVGSILGIH VGGNGRDGFS AMLLKSYFGE TQGLITKELP |
| m16248-89 | TKAGYCGGVL YKVGSILGIH VGGNGRDGFS AMLLKSYFGE TQGLITKELP |
| hrv-90 | TKSGYCGGIL YKIGQVLGIH VGGNGRDGFA AMLLRSYFSE TQGQITSTKT |
| hrv-94 | TKSGYCGGVL YKIGNILGIH VGGNGKDGFS ATLIRSYFSE TQGQITKRTS |
| ef173419-94 | TKSGYCGGVL YKIGNILGIH VGGNGKDGFS ATLIRSYFSE TQGQITKRTS |
| hrv-95 | TKSGFCGGVL YKVGQVIGIH VGGNGRQGFS AMLLKRYFNQ QQGQIIIKKP |
| hrv-96 | TKSGYCGGVL YKIGQIIGIH VGGNGRDGFS AMLLRSYFSE TQGEIKTTKK |
| hrv-98 | TKSGYCGGIL YKIGQVLGIH VGGNGRDGFA AMLLRSYFTE TQGQIVTTKS |
| hrv-100 | TKSGYCGGIL YKIGQVLGIH VGGNGRDGFA AMLLRSYFSE TQGQIITTKT |
| ef186077-qpm | TKPGQCGGVI LNTGNIIGIH VGGNGRVGYC ASLTKSYFAS AQGEIVNKCK |
| ef077279-nat001 | TKPGQCGGVI LNTGNIVGIH VGGNGRVGYC ASLTKSYFAS AQGEIVNKCK |
| ny1078 | TKPGQCGGVI LNTGNIIGIH VGGNGRVGYC ASLTKSYFAT TQGEIVNKCK |
| ef582385-c024 | TQPGQCGAVL ANTGNIIGIH VGGNGRVGYA AALLRKYFAE EQGAIISKCN |
| ef077280-nat045 | TKPGQCGGVL GNTGHIVGIH VGGNGRVGYG AALLRKYFCE QQGTITSIVD |
| ef582387-c026 | TKPGQCGGVV LNTGNIVGIH VGGNGRVGYC ASLTKSYFAS AQGEIINKCK |
| ef582386-c025 | TQPGQCGAVI ANVGNIIAIH VGGNGRVGYG AALLRKYFAA TQGEITTRKS |
| dq473485-03 | TKTGQCGGVL CTTGKIFGIH VGGNGRRGFS AQLKKQYFVE KQGLIVSKQK |
| ef173422-03 | TKTGQCGGVL CTTGKIFGIH VGGNGRQGFS AQLKKQYFVE KQGLIVSKQK |
| dq473490-04 | TKTGQCGGVL CSTGKIYGIH VGGNGRQGFS AQLKKQYFVQ KQGEIVQRKN |
| hrv-05 | TKTGQCGGVL CSTGKIYGIH VGGNGKQGFS AQLKKQYFVQ KQGQIITKRN |
| dq473486-06 | TKTGQCGGVL CTTGKIFGIH VGGNGRQGFS AQLRKQYFIE KQGQIISRQK |
| l05355-14 | TKTGQCGGVL CATGKIFGIH VGGNGRQGFS AQLKKQYFVE KQGQVIARHK |
| k02121-14 | TKTGQCGGVL CATGKIFGIH VGGNGRQGFS AQLKKQYFVE KQGQVIARHK |
| ef173420-17 | TRTGQCGGVL CSTGKIYGIH VGGNGRQGFS AQLRKQYFVE KQGAIINKRN |
| hrv-26 | TKTGQCGGVL CSTGKIYGIH VGGNGRQGFS AQLKKQYFVQ KQGQIVSKRN |
| hrv-27 | TKTGQCGGVL CSTGKIYGIH VGGNGRQGFS AQLKKQYFVQ KQGEIISKKK |
| ef173421-27 | TKTGQCGGVL CSTGKIYGIH VGGNGRQGFS AQLKKQYFVQ KQGEIISKKK |
| dq473487-35 | TKTGQCGGVL CTTGKIFGIH VGGNGRQGFS AQLRRQYFVS KQGQIVARQK |
| hrv-35 | TKTGQCGGVL CTTGKIFGIH VGGNGRQGFS AQLRRQYFVS KQGQIVARQK |
| ef173423-37 | TKTGQCGGVL CTTGKIFGIH VGGNGRQGFS AQLRRQYFIE KQGQIVSRQK |
| hrv-42 | TRTGQCGGVL CSTGKIYGIH VGGNGKQGFS AQLKKQYFVQ KQGQIITKRN |
| dq473488-48 | TKTGQCGGVL CSTGKIYGIH VGGNGRQGFS AQLKKQYFVE KQGAITTKRN |
| hrv-52 | TRTGQCGGVL CSTGKIYGIH VGGNGRQGFS AQLKKQYFVE KQGAITTRKN |
| hrv-52-f10 | TRTGQCGGVL CSTGKIYGIH VGGNGRQGFS AQLKRQYFVE KQGAITTRKN |
| ef173424-52 | TRTGQCGGVL CSTGKIYGIH VGGNGRQGFS AQLKKQYFVE KQGAITTRKN |
| hrv-69 | TRTGQCGGVL CSTGKIYGIH VGGNGRQGFS AQLKKQYFVE KQGAITTKRN |
| dq473489-70 | TRTGQCGGVL CSTGKIYGIH VGGNGRQGFS AQLRKQYFVE KQGAIINKRN |
| hrv-72 | TKTGQCGGVL CATGKIFGIH VGGNGRQGFS AQLKRQYFVE KQGQVIARQK |
| hrv-79 | TRTGQCGGVL CTTGKIFGIH VGGNGRQGFS AQLRRQYFVS KQGQITSRQK |
| hrv-83 | TRTGQCGGVL CTTGKIFGIH VGGNGRQGFS AQLRKQYFVS KQGQITSRQK |
| hrv-84 | TKTGQCGGVL CSTGKIYGIH VGGNGRQGFS AQLRKQYFVQ QQGQIISRKH |
| hrv-86 | TKTGQCGGVL CSTGKIFGIH VGGNGRQGFS AQLRKQYFVE KQGEIVSKQK |
| hrv-91 | TRTGQCGGVL CSTGKIYGIH VGGNGRQGFS AQLKKQYFVE KQGAIINKRS |
| hrv-92 | TRTGQCGGVL CTTGKIFGIH VGGNGRQGFS AQLRKQYFVA KQGQVIARQK |
| ef173425-93 | TKTGQCGGVL CSTGKIYGIH VGGNGKQGFS AQLKKQYFVQ KQGEIISKRK |
| hrv-97 | TKTGQCGGVL CSTGKIYGIH VGGNGKQGFS AQLKKQYFVQ KQGEIISKRK |
| hrv-99 | TKTGQCGGVL CSTGKIYGIH VGGNGRQGFS AQLKKQYFVQ KQGEIVTKRN |
| | 1801 1850 |
| annotation | .HHHH..... .......... .......... .......... ....HHHHHH |
| af499637-cva13 | SKEMGYPIIN APSKTKLEPS AFHYVFEGVK EPAVLTKNDP RLKTDFEEAI |
| af546702-cva21 | SKEVGYPIIN APSKTKLEPS AFHYVFEGVK EPAVLTKNDP RLKTDFEEAI |
| v01149-pv1m | SKEVGYPIIN APSKTKLEPS AFHYVFEGVK EPAVLTKNDP RLKTDFEEAI |
| d00239-01 | ANECGLPTIH TPSKTKLQPS VFYDVFPGSK EPAVSRDNDP RLKVNFKEAL |
| pico-01 | ATECGLPTIH TPSKTKLQPS VFYDVFPGSK EPAVLTDNDP RLEVNFKEAL |
| hrv-01 | ATECGLPTIH TPSKTKLQPS VFYDVFPGSK EPAVLTDNDP RLEVNFKEAL |
| x02316-02 | TSECNLPSIH TPCKTKLQPS VFYDVFPGSK EPAVLSEKDA RLQVDFNEAL |
| hrv-07 | VSSKNLPSIH VSSKTRLQPS VFYDVFPGTK EPAVLSSNDP RLEVDFDSAL |
| dq473503-07 | VSSKNLPSIH VSSKTRLQPS VFYDVFPGTK EPAVLSSNDP RLEVDFDSAL |
| hrv-08 | VKEVDYPSIH TPTKTKLQPS IFHDVFPGVK EPAVLSEKDP RLQVDLNTSL |
| pico-09 | VKDLGYPTIH TPSKTKLHPS VFHDVFKGTK EPAVLSEKDP RLEVDFKTSL |
| hrv-09 | VKDLGYPTIH TPSKTKLHPS VFHDVFKGTK EPAVLTEKDP RLEVDFKTSL |
| hrv-09-f01 | VKDIGYPTIH TPSKTKLHPS VFHDVFKGTK EPAVLSERDP RLEVDFKTSL |
| hrv-09-f02 | VKDIGYPTIH TPSKTKLHPS VFHDVFKGTK EPAVLSERDP RLEVDFKTSL |
| dq473498-10 | TSECGLPSIH TPSKTKLQPS VFFDVFPGTK EPAVLSSNDP RLEVDFNKAL |
| hrv-10 | TSECGLPSIH TPSKTKLQPS VFFDVFPGTK EPAVLSSNDP RLEVDFNKAL |
| ef173414-11 | VTELGLPSIH TPSKTKLQPS IFHDVFPGSK EPAVLSDNDP RLKVNFKEAL |
| ef173415-12 | VRNLAMPPIH VPTKTKLQPS VFHDIFPGVK EPAVLSKSDS RLETDFETAL |
| hrv-13 | VTECGLPTIH TPSKTKLQPS VFFDVFEGSK EPAVLTDRDP RLTTDFNKAL |
| hrv-13-f03 | VAECGLPTIH TPSKTKLQPS VFFDVFEGSK EPAVLTDKDP RLTTDFNKAL |
| dq473493-15 | VQECGYPTIH NPNKTRLHPS VFFEVFKGNK EPAVLSEKDP RLSVDFKTAL |
| l24917-16 | VKDVGLPSIH TPTKTKLQPS VFYDIFPGSK EPAVLTEKDP RLKVDFDSAL |
| hrv-18 | TAECNYPSIH TPSKTKLQPS VFYDIFPGNK EPAVLSEQDP RLKVDFKEAL |
| hrv-19 | VRERDLPTIH TPSKTKLQPS AFFDVFEGTK EPAVLSDRDP RLQTDFKTAL |
| hrv-20 | VKECNLYSIH TPSNTKLQPS VFYDVFPGSK EPAVLSGKDP RLEVDLDESI |
| hrv-21 | TDKCGLPSIH TPSKTKLQPS VFYDIFPGNK EPAVLSNKDP RLEVDFEKAL |
| hrv-22 | VRECGLPTIH TPSKTKLQPS VFYEVFEGTK EPAVLTDKDP RLETDFKTAL |
| dq473497-23 | TSECNLPSIH NPCKTKLQPS VFYDVFPGSK EPAVLSDKDT RLQVDFNESL |
| hrv-24 | TLDCNLPSIH TPSKTKLQPS VFYDVFPGNK EPAVLSEHDP RLEVNFKEAL |
| ef173416-24 | TLDCNLPSIH TPSKTKLQPS VFYDVFPGNK EPAVLSEHDP RLEVNFKEAL |
| hrv-25 | TSECGLPSIH TPNKTKLQPS VFYDVFPGSK EPAVLTSNDP RLEVDFKEAL |
| dq473508-28 | VKECNLYSIH TPSNTKLQPS VFHDIFPGSK EPAVLSSKDP RLEVNLDESI |
| hrv-29 | TSECGLPSIH TPSKTKLQPS VFYDVFPGNK EPAVLSSNDP RLKVDFKEAL |
| dq473512-30 | TSECNLPSIH NPCKTKLQPS VFYDVFPGSK EPAVLSEKDT RLQVDFNEAL |
| hrv-30 | TSECNLPSIH NPCKTKLQPS VFYDVFPGSK EPAVLSEKDT RLQVDFNEAL |
| hrv-31 | TIECNLPSIH TPSKTKLQPS VFYDVFPGSK EPAVLSEHDS RLEVDFKQAL |
| hrv-32 | VKDLGYPTIH TPNKTKLHPS VFYDVFKGTK EPAVLSEKDP RLETDFKTAL |
| hrv-33 | VVELGLPSIH TPSKTKLQPS VFHDVFPGSK EPAVLSDNDP RLEVDFKQAL |
| hrv-34 | TAECNLPSIH TPSKTKLQPS VFYDVFPGNK EPAVLSDHDP RLKVNFSEAL |
| dq473501-34 | TAECNLPSIH TPSKTKLQPS VFYDVFPGNK EPAVLSDHDP RLKVNFSEAL |
| dq473505-36 | VSARNLPSIH VSSKTRLQPS VFHDVFPGTK EPAVLSNNDP RLETDFDTAL |
| hrv-38 | TSECNLPSIH TPSKTKLQPS VFHDVFPGSK EPAVLSEHDP RLKVNFKEAI |
| dq473495-38 | TSECNLPSIH TPSKTKLQPS VFHDVFPGSK EPAVLSEHDP RLKVNFKEAI |
| ay751783-39 | TSECGLPSIH NPSKTKLQPS VFYDIFPGSK QPAVLSEKDT RLQVDFNEAL |
| hrv-40 | TIECNLPSIH TPSKTKLQPS VFYDVFPGNK EPAVLSEHDP RLKVDFKEAL |
| dq473491-41 | VSELNLPTIH TPAKTKLQPS VFFDVFEGTK EPAVLTERDP RLTTDFNTAL |
| hrv-43 | VREYGFPTIH TPSKTKLQPS VFFDVFEGSK EPAVLSDKDP RLETDFKTAL |
| dq473499-44 | TSECGVPSIH TPSKTKLQPS VFYDVFPGNK EPAVLSSNDP RLKVDFKEAL |
| hrv-45 | VKEVDYPSIH TPTKTKLQPS VFHDIFPGVK EPAVLTEKDP RLEVDLNSSL |
| dq473506-46 | VKEYNMYSIH TPTKTKLQPS VFHDVFPGSK EPAVLSTKDP RLEVDLDSSI |
| hrv-47 | TIECNLPSIH TPSKTKLQPS VFYDVFPGSK EPAVLSEYDP RLEVDFKQAL |
| dq473496-49 | TSECNLPSIH NPSKTKLQPS VFYDVFPGSK EPAVLSEKDT RLQVDFNEAL |
| hrv-49-f04 | TSECNLPSIH SPSKTKLQPS VFYDVFPGSK EPAVLSEKDA RLQVDFNEAL |
| hrv-50 | TIECNLPSIH TPSKTKLQPS VFYDVFPGNK EPAVLSNHDP RLKVDFNEAL |
| hrv-51 | VKEKNLYSIH TPTKTKLQPS IFHDVFPGKK EPAVLSSKDP RLQVDLEEAI |
| dq473507-53 | VKEHNLYSIH TPTKTKLQPS VFHDVFPGSK EPAVLSTRDP RLEVDLDSSI |
| hrv-54 | TIECNLPSIH TPSKTKLQPS VFYDVFPGSK EPAVLSEHDP RLEVDFKQAL |
| hrv-54-f05 | TIECNLPSIH TPSKTKLQPS VFYDVFPGSK EPAVLSEHDP RLEVDFKQAL |
| dq473511-55 | TSECGLPSIH TPSKTKLQPS VFYDVFPGNK EPAVLSNHDP RLEVDFEKAI |
| hrv-56 | TSECGLPSIH TPSKTKLQPS VFFDVFPGTK EPAVLTNSDH RLEVDFNQAL |
| hrv-57 | TDKCGLPSIH TPSKTKLQPS VFYDVFPGSK EPAVLSSNDP RLEVDFEKAL |
| hrv-58 | VSVKNLPSIH VSSKTRLQPS VFHDVFPGTK EPAVLSNNDP RLETDFDTAL |
| dq473500-59 | TSLCGLPSIH TPSKTKLQPS VFHDIFPGSK EPAVLSSKDP RLEVDFDEAL |
| hrv-60 | TAECNLPSIH TPSKTKLQPS VFYNVFPGNK EPAVLSEHDP RLKTNFKEAL |
| hrv-61 | VHECGYPTIH TPAKTKLQPS VFFDVFEGSK EPAVLTEKDP RLTTDFNQAL |
| hrv-62 | TSECGLPSIH TPNKTKLQPS VFYDVFPGSK EPAVLTSNDP RLEVDFKEAL |
| hrv-63 | AKECGLPSIH TPSKTKLQPS VFYDVFPGTK EPAVLSSNDP RLEVDFNEAL |
| hrv-64 | VNECGLPTIH TPSKTKLQPS VFYDVFEGTK EPAVLTDKDP RLETDFKTAL |
| ef173417-64 | VNECGLPTIH TPSKTKLQPS VFYDVFEGTK EPAVLTDKDP RLETDFKTAL |
| hrv-65 | VREKNLFSIH TPTKTKLQPS VFHDTFPGKK EPAVLSSKDP RLQVDLEESI |
| hrv-66 | TIECNLPSIH TPSKTKLQPS IFYDVFPGNK EPAVLSDHDP RLEVDFKEAL |
| hrv-67 | VKDLGYPTIH TPNKTKLQPS VFHDVFKGTK EPAVLSDKDP RLETDFRTSL |
| hrv-68 | VKECNLYSIH TPSNTKLQPS VFYDVFPGSK EPAVLSGKDP RLEVNLDESI |
| hrv-71 | VKDKNLYSIH TPTNTKLQPS VFHDTFPGKK EPAVLSPKDP RLQVNLEEAI |
| dq473492-73 | VAECGLPTIH TPSKTKLQPS VFFDVFEGSK EPAVLTDKDP RLTTDFNKAL |
| dq473494-74 | VQDCGYPTIH NPTKTKLLPS VFFDVFKGSK EPAVLSEKDP RLETDFRTAL |
| dq473510-75 | VRNIGYPTIH TPSKTKLQPS VFFDVFEGTK EPAVLSESDP RLKTDFKTAL |
| dq473502-76 | TVELGLPSIH TPSKTKLQPS VFHDVFPGSK EPAVLSDNDP RLEVDFKQAL |
| hrv-76 | TVELGLPSIH TPSKTKLQPS VFHDVFPGSK EPAVLSDNDP RLEVDFKQAL |
| hrv-77 | TTECNLPSIH TPSKTKLQPS IFYDIFPGNK EPAVLSENDP RLEVNFKEAL |
| hrv-78 | VSVAGFNPVH VPSKTKLQPS IFFDVFPGDK EPAVLSKHDP RLETDFHTAL |
| ef173418-78 | VSVAGFNPVH VPSKTKLQPS IFFDVFPGDK EPAVLSKHDP RLETDFHTAL |
| hrv-80 | TKEKGIYSIH TPTKTKLQPS VFHDVFPGTK EPAVLSPYDN RLNVDLEDSI |
| hrv-81 | VKDVGLPSIH TPSKTKLQPS VFYDVFPGSK EPAVLTDKDP RLKVDFETAL |
| hrv-81-f06 | VKDVGLPSIH TPSKTKLQPS VFYDIFPGSK EPAVLTDKDP RLKVDFETAL |
| hrv-81-f07 | VKDVGLPSIH TPSKTKLQPS VFYDIFPGSK EPAVLTDKDP RLKVDFETAL |
| hrv-82 | VREYGLPTIH TPSKTKLQPS VFHDVFKGTK EPAVLSEKDP RLETDFKKAL |
| dq473509-82 | VKEHGFPTIH TPSKTKLQPS VFYDVFKGTK EPAVLSEKDP RLETDFKKAL |
| hrv-85 | TTECNYPSVH TPSKTKLQPS VFYDVFPGNK EPAVLSEHDP RLKVDFKEAL |
| pico-85 | TTECNYPSVH TPSKTKLQPS VFYDVFPGNK EPAVLSEHDP RLKVDFKEAL |
| dq473504-88 | VSAKNLPSIH VSSKTRLQPS VFHDVFPGTK EPAVLSDRDP RLEVDFNSAL |
| a10937-89 | VSVKNLPSVH VSSKTRLQPS VFHDVFPGTK EPAVLSSNDP RLETDFDSAL |
| hrv-89 | VSVKNLPSIH VSSKTRLQPS VFHDVFPGTK EPAVLSSNDP RLETDFDSAL |
| hrv-89-f08 | VSVKNLPSIH VSSKTRLQPS VFHDVFPGTK EPAVLSSNDP RLETDFDSAL |
| hrv-89-f09 | VSVKNLPSIH VSSKTRLQPS VFHDVFPGTK EPAVLSSNDP RLETDFDSAL |
| m16248-89 | VSVKNLPSVH VSSKTRLQPS VFHDVFPGTK EPAVLSSNDP RLETDFDSAL |
| hrv-90 | THECALPSIH TPSKTKLQPS VFYDVFPGSK EPAVLSDHDP RLEVDFKEAL |
| hrv-94 | TKEYGLPTIH TPSKTKLQPS VFYDVFEGTK EPAVLTDKDP RLETDFKTAL |
| ef173419-94 | TKEYGLPTIH TPSKTKLQPS VFYDVFEGTK EPAVLTDKDP RLETDFKTAL |
| hrv-95 | VKEVDYPSIH TPTKTKLQPS IFHDVFPGVK EPAVLSEKDP RLQVDLNTSL |
| hrv-96 | VNECGYPTIH TPAKTKLQPS VFFDVFEGSK EPAVLSEKDP RLKTDFNQAL |
| hrv-98 | TIECNLPSIH TPSKTKLQPS VFYDVFPGSK EPAVLSEHDP RLEVDFKQAL |
| hrv-100 | TLECNLPSIH TPSKTKLQPS VFYDVFPGSK EPAVLSSSDP RLEVDFKEAL |
| ef186077-qpm | VQDVGLKPIN TPEHSKLHPS VFYDIFPGEK EPAVLTQRDP RLETDLKTAV |
| ef077279-nat001 | VQDVGLKPIN TPEHSKLYPS VFYDIFPGEK EPAVLTQKDP RLETDLKTAV |
| ny1078 | VQDVGLKPIN TPEHSKLYPS VFFHVFPGEK EPAALTQKDP RLETDLKTAV |
| ef582385-c024 | VKEKNWKPVN TPSQTKLHPS VFHDVFPGVK EPAALHPKDP RLEVDLTTAV |
| ef077280-nat045 | VRELGYSPIN TPAKTKLLPS VFYDIFPGIK EPAPLSNRDS RLESNLDDTV |
| ef582387-c026 | VQDVGLKPIN TPEHSKLHPS VFYDIFPGEK EPAVLTQKDP RLETDLKTAV |
| ef582386-c025 | VRDVGMSPIN TPCKTKLYPS VFYDIFPGKK EPAALSDKDP RLEVNLKEAV |
| dq473485-03 | VRDIGLNPIN TPTKTKLHPS VFYNVFPGSK QPAVLNDNDP RLEVKLAESL |
| ef173422-03 | VRDIGLNPIN TPTKTKLHPS VFYNVFPGSK QPAVLNDNDP RLEVKLAESL |
| dq473490-04 | VRELDIKPIN TPSKTKLHPS VFYNVFPGTK EPAVLHPSDS RLEVDFEESL |
| hrv-05 | VRELDIKPIN TPSKTKLHPS VFYNVFPGSK EPAVLHPADE RLEVNFEESL |
| dq473486-06 | VRELNMASIN TPTKSKLHPS VFYNVFPGSK EPAVLSDNDP RLEVKLSDSL |
| l05355-14 | VREFNINPVN TPTKSKLHPS VFYDVFPGDK EPAVLSDNDP RLEVKLTESL |
| k02121-14 | VREFNINPVN TATKSKLHPS VFYDVFPGDK EPAVLSDNDP RLEVKLTESL |
| ef173420-17 | VKELGLKPIN TPTKTKLHPS VFYETFPGTK EPAALNDSDP RLEVNLRDSL |
| hrv-26 | VRELNIKPIN TPNTTKLHPS VFYNVFPGSK EPAVLHPADQ RLEVNFEDSL |
| hrv-27 | VHDLGLKPIN TPSKTKLLPS VFYNVFPGTK EPAVLHPADE RLEVDFEESL |
| ef173421-27 | VHDLGLKPIN TPSKTKLLPS VFYNVFPGTK EPAVLHPADE RLEVDFEESL |
| dq473487-35 | LKDLNLMPVN TPTKTKIHPS VFYDVFPGSK EPAVLSDNDP RLEVKLSESL |
| hrv-35 | LKDLNLMPVN TPTKTKIHPS VFYDVFPGSK EPAVLSDNDP RLEVKLSESL |
| ef173423-37 | VRELNIAPVN TPNKTKLHPS VFYNVFPGSK EPAVLSDNDP RLEVKLSDSL |
| hrv-42 | VRELSIKPIN TPNTTKLHPS VFYNVFPGSK EPAVLHPADE RLEVNFEESL |
| dq473488-48 | VREVGLKPIN TPTKTKLHPS VFYEIFPGSK EPAVLDDHDP RLEVNLHDSL |
| hrv-52 | VREVGLKPIN TPTKTKLHPS VFHEIFPGSK EPAVLNDTDP RLEVNLCDSL |
| hrv-52-f10 | VREMGLKPIN TPTKTKLHPS VFYEVFPGSK EPAVLNDADP RLEVNLRDSL |
| ef173424-52 | VREVGLKPIN TPTKTKLHPS VFHEIFPGSK EPAVLNDTDP RLEVNLCDSL |
| hrv-69 | VREVGLKPIN TPTKTKLHPS VFYEVFPGSK EPAVLDDSDP RLEVNLRDSL |
| dq473489-70 | VKELGLKPIN TPTKTKLHPS VFYEIFPGSK EPAALNDSDP RLEVNLRDSL |
| hrv-72 | VRELNMNPIN TPTKSKLHPS VFYNVFPGDK EPAVLSDNDP RLEVKLTESL |
| hrv-79 | LKDLNLNPIN TPSKTKLHPS VFYDIFPGNK QPAVLSDNDP RLEVKLADSL |
| hrv-83 | LKDLNLNPVN TPTKTKLHPS VFHDVFPGSK QPAVLSDNDP RLEVKLTESL |
| hrv-84 | VDDLNIKPIN TPTKTKLLPS VFYNVFPGSK EPAVLHPADK RLEVDLNDSL |
| hrv-86 | VKELGLHPIN TPSKTKLHPS VFYNVFEGNK QPAVLKDNDP RLEVNFSESL |
| hrv-91 | VRELGLKPIN TPTKTKLHPS VFYDVFPGSK EPAALNGSDP RLEVNLRDSL |
| hrv-92 | LRELNLAPVN TPTKTKLHPS VFYDVFPGNK QPAVLSDNDP RLEVKLTESL |
| ef173425-93 | VQELGMKPIN TPNKTKLLPS VFYNVFPGTK EPAVLHPADE RLEVNFEESL |
| hrv-97 | VHDLGLKPIN TPSKTKLLPS VFYNVFPGTK EPAVLHPSDE RLEVDFEESL |
| hrv-99 | VRELNLKPIN TPSKTKLHPS VFFNVFPGSK EPAVLHPSDE RLEVIFEDSL |
| | 1851 1900 |
| annotation | HH........ ..HHHHHHHH HHHHHHHHH. .......HHH HHHHH..... |
| af499637-cva13 | FSKYVGNKIT EVDEYMKEAV DHYAGQLMSL DINTEQMCLE DAMYGTDGLE |
| af546702-cva21 | FSKYVGNKIT EVDEYMKEAV DHYAGQLMSL DINTEQMCLE DAMYGTDGLE |
| v01149-pv1m | FSKYVGNKIT EVDEYMKEAV DHYAGQLMSL DINTEQMCLE DAMYGTDGLE |
| d00239-01 | FSKYKGNTEC SLNQHMEIAI AHYSAQLITL DIDSKPIALE DSVFGIEGLE |
| pico-01 | FSKYKGNVEC NLNEHMEIAI AHYSAQLMTL DIDSRPIALE DSVFGIEGLE |
| hrv-01 | FSKYKGNVEC NLNEHMEIAI AHYSAQLMTL DIDSRPIALE DSVFGIEGLE |
| x02316-02 | FSKYKGNTDC SINDHIRIAS SHYAAQLITL DIDPKPITLE DSVFGTDGLE |
| hrv-07 | FSKYKGNVNC QLTPHMEIAV AHYAAQLSTL DINPQPLSLE ESVFGIDGLE |
| dq473503-07 | FSKYKGNVNC QLTPHMEIAV AHYAAQLSTL DINPQPLSLE ESVFGIDGLE |
| hrv-08 | FSKYAGNKQM EVNEYMKVAA SHYASQLETL DIPNQQMSIE DCVYGTDNLE |
| pico-09 | FSKYKGNVDC TLNEHMRVAI AHYSAQLMTL DINPDNITME ESVFGTDGLE |
| hrv-09 | FSKYKGNVDC TLNEHMRVAI AHYSAQLMTL DINPDNITME ESVFGTDGLE |
| hrv-09-f01 | FSKYKGNVDC TLNEHMRVAI AHYSAQLMTL DINPNNITME ESVFGTDGLE |
| hrv-09-f02 | FSKYKGNVDC TLNEHMRVAI AHYSAQLMTL DINPNNITME ESVFGTDGLE |
| dq473498-10 | FSKYKGNNEC VINDHVKVAV SHYSAQLMTL DINPEPISLE DSVFGTEGLE |
| hrv-10 | FSKYKGNNEC VINDHVKVAV SHYSAQLMTL DINPEPISLE DSVFGTEGLE |
| ef173414-11 | FSKYKGNNDC VMNDHIKVAV SHYSAQLLTL DIDSKPITVE DSVFGIDGLE |
| ef173415-12 | FSKYKGNVHM DLTPHMEVAI SHYTAQLSSL DIPNTELSLE ECVYGAEGLE |
| hrv-13 | FSKYKGNIEC SMTDHMRVAV SHYSAQLMTL DIDSVNISLE ESVFGTEGLE |
| hrv-13-f03 | FSKYKGNIEC SMTDHMRVAI SHYSAQLMTL DIDSVNISLE ESVFGTEGLE |
| dq473493-15 | FSKYKGNNTC ELNDHIKVAV AHYSAQLATL DIGPASMSLE DSVFGTDGLE |
| l24917-16 | FSKYKGNTEC SLNEHIQVAV AHYSAQLATL DIDPQPIAME DSVFGMDGLE |
| hrv-18 | FSKYKGNNPC EINEHIKVAV SHYSAQLMTL DIDPSPISLE DGVFGMEGLE |
| hrv-19 | FSKYKGNKKF EMNDHIRVAV SHYSAQLMTL DIDHSNMSLE DSVFGIEGLE |
| hrv-20 | FSKYKGNEPV EITQNMLTAT AHYTAQLSTL DINPKPLSLE ESVYGIEGLE |
| hrv-21 | FSKYKGNEHC VMNDHINVAI SHYSAQLATL DINPQPISIE ESVFGMDGLE |
| hrv-22 | FSKYKGNLPC EMNDHVKVAV AHYSAQLMTL EIDSRNMSLE DSVFGTEGLE |
| dq473497-23 | FSKYKGNTHC VMNEHIKIAS AHYAAQLITL DIDPNPITLE DSVFGTDGLE |
| hrv-24 | FSKYKGNSQC ELNDHIKVAI SHYSAQLTTL DICPDPISLE DSVFGVEGLE |
| ef173416-24 | FSKYKGNSQC ELNDHIKVAI SHYSAQLTTL DICPDPISLE DSVFGVEGLE |
| hrv-25 | FSKYKGNNQC TMNDHIKVAI SHYSAQLMTL DIDPKPISLE ESVFGIEGLE |
| dq473508-28 | FSKYKGNEPV EITQNMLTAA AHYTAQLSTL DIDSTPLSLE ESVYGIEGLE |
| hrv-29 | FSKYKGNNQC TMNDHIRVAI SHYSAQLMTL DIDPKPISLE ESVFGIEGLE |
| dq473512-30 | FSKYKGNTHC TMNDHMKIAS SHYAAQLITL DINPSPITLE DSVFGTDGLE |
| hrv-30 | FSKYKGNTHC TMNDHMKIAS SHYAAQLITL DINPSPITLE DSVFGTDGLE |
| hrv-31 | FSKYKGNSQC EINDHVKVAI SHYSAQLMTL DIDPNPISLE DSVFGAEGLE |
| hrv-32 | FSKYKGNVNC ELNDHMRVAI AHYSAQLMTL DIDPSNMTME ESVFGTDGLE |
| hrv-33 | FSKYKGNNEC AMNEHIKVAV SHYSAQLHTL DVDPRPITVE DSVFGVEGLE |
| hrv-34 | FSKYKGNNQC TINDHIKVAI SHYSAQLTTL DIDPNPITLE ESVFGIEGLE |
| dq473501-34 | FSKYKGNNQC TINDHIKVAI SHYSAQLTTL DIDPNPITLE ESVFGIEGLE |
| dq473505-36 | FSKYKGNPAC EVTTHMKIAV AHYAAQLSTL DINPQPLSLE ESVFGIEGLE |
| hrv-38 | FSKYKGNNQC EMNDHIRVAI SHYSAQLATL DIDPNPISLE DSVFGIEGLE |
| dq473495-38 | FSKYKGNNQC EMNDHIRVAI SHYSAQLATL DIDPNPISLE DSVFGIEGLE |
| ay751783-39 | FSKYKGNVDC PMNDHIKIAS SHYAAQLITL DINPNPITLE DGVFGTEGLE |
| hrv-40 | FSKYKGNNPC EVNDHIKVAI SHYSAQLMTL DIDSNPISLE DSVFGIEGLE |
| dq473491-41 | FSKYKGNVKC EMSEHMKVAI SHYSAQLMTL DIDPTKISLE ESVFGTEGLE |
| hrv-43 | FSKYKGNKEC EMNDHIKVAV AHYSAQLMTL DIDHSNMSLG DSVFGIEGLE |
| dq473499-44 | FSKYKGNNQC IMNDHIKVAI SHYSAQLMTL DIDPKPISLE ESVFGIEGLE |
| hrv-45 | FSKYAGNVNL EMNEYMIVAA SHYASQLETL DISNQQMSIE ECVYGTDNLE |
| dq473506-46 | FSKYKGNEAI KITDNMLVAA AHYTAQLSTL DIDPQPISLE DSVYGIEGLE |
| hrv-47 | FSKYKGNNQC EINDHVRVAI SHYSAQLMTL DIDSNPISLE DSVFGTEGLE |
| dq473496-49 | FSKYKGNTNC FMNDHIRIAS SHYAAQLITL DIDPSPITLE DSVFGTDGLE |
| hrv-49-f04 | FSKYKGNTNC SMNDHIRIAS SHYAAQLITL DIDPSPITLE DSVFGTDGLE |
| hrv-50 | FSKYKGNNHC TMNDHIKVAI SHYSAQLATL DINPSPISIE DSVFGIEGLE |
| hrv-51 | FSKYKGNKEI KITQNMEVAA AHYAAQLSTL NINPEPLTME ESVYGIEGLE |
| dq473507-53 | FSKYKGNEAV KISENMLVAA AHYTAQLTTL DIDPQPISLE DSVYGIEGLE |
| hrv-54 | FSKYKGNNQC EMNEHIKVAA SHYSAQLMTL DIDSNPISLE DSVFGTEGLE |
| hrv-54-f05 | FSKYKGNNQC EINEHIKVAA SHYSAQLMTL DIDSNPISLE DSVFGTEGLE |
| dq473511-55 | FSKYKGNEQC VINDHIKVAV SHYSAQLATL DINPQPVSIE ESVFGMEGLE |
| hrv-56 | FSKYKGNNQC VMNDHVKVAI SHYSAQLMTL DINPEPISLE DSVFGTEGLE |
| hrv-57 | FSKYKGNEHC VMNDHINVAV SHYSAQLATL DINPQPISIE ESVFGIEGLE |
| hrv-58 | FSKYKGNPTC EVTPHMKIAV AHYAAQLSTL DISTQPLSLE ESVFGIDGLE |
| dq473500-59 | FSKYKGNVDC PMNDHIRVSV AHYSAQLATL DIDPNPVSME EGVFGMEGLE |
| hrv-60 | FSKYKGNNQC EINDHIRVAI SHYSAQLATL DIDPSPISLE DSVFGIEGLE |
| hrv-61 | FSKYKGNVEC NMSEHMKVAI SHYSAQLMTL DIDPSNITLE ESVFGTEGLE |
| hrv-62 | FSKYKGNNQC TINDHIKVAI SHYSAQLMTL DIDSKPISLE ESVFGIEGLE |
| hrv-63 | FSKYKGNNEC AINNHVKVAI SHYSAQLMTL DINPEPISLE DSVFGIEGLE |
| hrv-64 | FSKYKGNLPC EMNDHVKVAV AHYSAQLMTL EIDSRNMSLE DSVFGADGLE |
| ef173417-64 | FSKYKGNLPC EMNDHVKVAV AHYSAQLMTL EIDSRNMSLE DSVFGADGLE |
| hrv-65 | FSKYKGNKQI EITQNMRVAA AHYTAQLSTL DISPEPLSME ESVYGIEGLE |
| hrv-66 | FSKYKGNNKC ELNDHIKVAT SHYAAQLATL DIDSNPITLE DSVFGVEGLE |
| hrv-67 | FSKYKGNVEC ELNDHMKVAI AHYSAQLMTL DINPSNITIE DSVFGTDGLE |
| hrv-68 | FSKYKGNELV EITQNMLTAA AHYTAQLSTL DIDPTPLSLE ESVYGIEGLE |
| hrv-71 | FSKYKGNEKI EITQNMKIAA AHYAAQLSTL DINSEPLTLE ESVYGIEGLE |
| dq473492-73 | FSKYKGNIEC SMTDHMRVAI SHYSAQLMTL DIDSTNMSLE ESVFGTEGLE |
| dq473494-74 | FSKYKGNNTC QMNDHIRVAV AHYSAQLATL DINPANITLE DSVFGIEGLE |
| dq473510-75 | FSKYKGNPEC EMNDHIKVAI AHYSAQLMTL DIQTSSVSLE ESVFGIDGLE |
| dq473502-76 | FSKYKGNNEC TMNEHIKVAV SHYSAQLHTL DIDPKSITVE DSVFGIEGLE |
| hrv-76 | FSKYKGNNEC TMNEHIKVAV SHYSAQLHTL DIDPKSITVE DSVFGIEGLE |
| hrv-77 | FSKYKGNNPC EXNDHIKVAV SHYSAQLMTL DIDSNPISLE SSVFGIEGLE |
| hrv-78 | FSKYSGNQQI EMSDNIKVAV AHYSAQLSTL DINPAPLSLE ECVFGIDGLE |
| ef173418-78 | FSKYSGNQQI EMSDNIKVAV AHYSAQLSTL DINPAPLSLE ECVFGIDGLE |
| hrv-80 | FSKYKGNTSL EVTHNMKVAA THYAAQLMTL DIDPKPLTLE ESVYGIEGLE |
| hrv-81 | FSKYKGNVKC DLNDHMQVAV AHYSAQLATL DIDPQPIALE DSVFGMDGLE |
| hrv-81-f06 | FSKYKGNVEC ELNDHIQVAV AHYSAQLATL DIDPQPVTLE DSVFGMDGLE |
| hrv-81-f07 | FSKYKGNVEC ELNDHIQVAV AHYSAQLATL DIDPQPVTLE DSVFGMDGLE |
| hrv-82 | FSKYKGNLDC EMNDHVKVAV AHYSAQLMTL DIDSSNMSLE DSVFGSEGLE |
| dq473509-82 | FSKYKGNLNC EINDHVKIAV AHYSAQLMTL DIDSNNMSLE DSVFGSDGLE |
| hrv-85 | FSKYKGNGPC EMNDHIKVAI SHYSAQLMTL DIDSNPISLE DSVFGMEGLE |
| pico-85 | FSKYKGNGPC EMNDHIKVAI SHYSAQLMTL DIDSNPISLE DSVFGMEGLE |
| dq473504-88 | FSKYKGNADC PLTSHMKVAV AHYAAQLSTL DINPQPLSLE ESVFGIEGLE |
| a10937-89 | FSKYKGNPAC QVTPHMKIAV AHYAAQLSTL DINPQPLSLE ESVFGIEGLE |
| hrv-89 | FSKYKGNPAC QVTPHMKIAV AHYAAQLSTL DINPQPLSLE ESVFGIEGLE |
| hrv-89-f08 | FSKYKGNPAC QVTPHMKIAV AHYAAQLSTL DINPQPLSLE ESVFGIEGLE |
| hrv-89-f09 | FSKYKGNPAC QVTPHMKIAV AHYAAQLSTL DINPQPLSLE ESVFGIEGLE |
| m16248-89 | FSKYKGNPAC QVTPHMKIAV AHYAAQLSTL DINPQPLSLE ESVFGIEGLE |
| hrv-90 | FSKYKGNNQC VMNDHIKVAI SHYSAQLMTL DIDPKPISIE DSVFGIEGLE |
| hrv-94 | FSKYKGNLPC EMNDHVKIAV AHYSAQLMTL DIDPRNMTLE DSVFGADGLE |
| ef173419-94 | FSKYKGNLPC EMNDHVKIAV AHYSAQLMTL DIDPRNMTLE DSVFGADGLE |
| hrv-95 | FSKYAGNKQM EVNEYMKVAA SHYASQLETL DIPNQQMSIE DCVYGTDNLE |
| hrv-96 | FSKYKGNVNC DMSEHMKVAV SHYSAQLMTL DIDPSNITLE ESVFGTEGLE |
| hrv-98 | FSKYKGNNQC EMNEHIKVAV SHYSAQLMTL DIDSNPISLE DSVFGTEGLE |
| hrv-100 | FSKYKGNEHC VLNDHIRVAV SHYSAQLITL DIDPKPISLE DSVFGIEGLE |
| ef186077-qpm | MSKYKGNVKI EMSENIQIAV EHYSAQLFML DINPEPISLE QAVYGMENLE |
| ef077279-nat001 | LSKYKGNVQI EMNENIQIAV EHYSAQLFML DINPEPISLE QAVYSMQNLE |
| ny1078 | MSKYKGNVHI EMSENIHIAV EHYSAQLFML DINPEPITLE QAVYGMQNLE |
| ef582385-c024 | MSKYKGNIDV PMNEYIETAV DHYAAQLYML DINPEPITME QAIYGYANLE |
| ef077280-nat045 | FSKYKGNAQV EFNEFMQVAV DHYSAQLYTL DINPAPLTLE QAAYGIPNLE |
| ef582387-c026 | LSKYRGNVEI EMSENMQIAV EHYSAQLFML DINPEPISLE QAVYGMENLE |
| ef582386-c025 | MSKYKGNKGC QMNDYVLTAA KHYAAQLFPL DINPEPITME QAVYGTDYLE |
| dq473485-03 | FSKYKGNVQM EPTENMLIAV DHYAGQLMSL DISTKELTLK EALYGVDGLE |
| ef173422-03 | FSKYKGNVQM EPTENMLIAV DHYAGQLMSL DISTKELTLK EALYGVDGLE |
| dq473490-04 | FSKYKGNAKM EITDNMRVAI DHYSGQLLSL DIPTQELTLK EALYGTDGLE |
| hrv-05 | FSKYKGNIES EITENMKVAI DHYAGQLLSL DIPTQELTLK EALYGTDGLE |
| dq473486-06 | FSKYKGNIEM KPTENMLIAV DHYAGQLMSL DIPTEELTLK EALYGVDGLE |
| l05355-14 | FSKYKGNVNT EPTENMLVAV DHYAGQLLSL DIPTSELTLK EALYGVDGLE |
| k02121-14 | FSKYKGNVNT EPTENMLVAV DHYAGQLLSL DIPTSELTLK EALYGVDGLE |
| ef173420-17 | FSKYKGNVEI DITENMQIAI DQYSGQLLSL DIPTNELSLK EALYGTDGLE |
| hrv-26 | FSKYKGNVDM EISDNMKVAI DHYAGQLLSL DIPTQELSLK EALYGTDGLE |
| hrv-27 | FSKYKGNVKM EISENMTIAI DHYAGQLLSL DIPTKELSLK EALYGTDGLE |
| ef173421-27 | FSKYKGNVKM EISENMTIAI DHYAGQLLSL DIPTKELSLK EALYGTDGLE |
| dq473487-35 | FSKYKGNVEM KPTDNMLVAV DHYAGQLMSL DIPTTELTLK EALYGVDGLE |
| hrv-35 | FSKYKGNVEM KPTDNMLVAV DHYAGQLMSL DIPTTELTLK EALYGVDGLE |
| ef173423-37 | FSKYKGNVEM KPTENMLVAV DHYAGQLMSL DIPTEELTLK EALYGVDGLE |
| hrv-42 | FSKYKGNVKM EITENMKVAI DHYAGQLLSL DIPTQELTLK EALYGTDGLE |
| dq473488-48 | FSKYKGNVET NITENMQIAI DQYSGQLLSL DIPTEELTLK DALYGTDGLE |
| hrv-52 | FSKYKGNVEI DITENMQVAI DQYSGQLLSL DIPTEELSLK EALYGTDGLE |
| hrv-52-f10 | FSKYRGNVET DITENMQVAI DQYSGQLLSL DISTDELSLK EALYGTDGLE |
| ef173424-52 | FSKYKGNVEI DITENMQVAI DQYSGQLLSL DIPTEELSLK EALYGTDGLE |
| hrv-69 | FSKYKGNVEV DITENMQVAI DHYSGQLLSL DIPTDEMSLK EALYGTDGLE |
| dq473489-70 | FSKYKGNVEI DITENMQIAI DQYSGQLLSL DIPTDELSLK EALYGTDGLE |
| hrv-72 | FSKYKGNVEM EPTENMLVAV DHYAGQLLSL DIPTEELTIK EALYGVDGLE |
| hrv-79 | FSKYKGNVEM KPTDNMLVAI DHYAGQLMSL DIPTNELTLK EALYGVDGLE |
| hrv-83 | FSKYKGNVEM KPTENMLIAV DHYAGQLMSL DIPTGELSLK EALYGVDGLE |
| hrv-84 | FSKYKGNVNM KMTENMKVAV DHYAGQLMSL EIPTEELSLK EALYGTDGLE |
| hrv-86 | FSKYKGNVEM KPTPNMIVAV DHYAGQLMSL DIPVKELSLK EALYGVDGLE |
| hrv-91 | FSKYKGNIEI DITENMQIAI DQYSGQLLSL DIPTNELSLK EALYGTDGLE |
| hrv-92 | FSKYKGNVEM KPTENMLVAV DHYAGQLLSL DIPTDELSLK EALYGVDGLE |
| ef173425-93 | FSKYKGNVKM EISDNMIIAV DHYAGQLLSL DIPTRELSLK EALYGTDGLE |
| hrv-97 | FSKYKGNVKI EITENMCIAI DHYAGQLLSL DIPTKELSLK EALYGTDGLE |
| hrv-99 | FSKYKGNVKM EMTENMKVAV DHYAGQLLSL DIPTQELTLK EALYGTDGLE |
| | 1901 1950 |
| annotation | .......... HHHHH..HHH HH........ HHHHHHHHHH H....BBBBB |
| af499637-cva13 | ALDLSTSAGY PYVAMGKKKR DILNKQTRDT KEMQRLLDTY GINLPLVTYV |
| af546702-cva21 | ALDLSTSAGY PYVAMGKKKR DILDKQTRDT KEMQRLLDTY GINLPLVTYV |
| v01149-pv1m | ALDLSTSAGY PYVAMGKKKR DILNKQTRDT KEMQKLLDTY GINLPLVTYV |
| d00239-01 | ALDLNTSAGF PYVTMGIKKR DLINNKTKDI SRLKEALDKY GVDLPMITFL |
| pico-01 | ALDLNTSAGF PYVTMGIKKR DLINNKTKDI SRLKEALDKY GVDLPMITFL |
| hrv-01 | ALDLNTSAGF PYVTMGIKKR DLINNKTKDI SRLKEALDKY GVDLPMITFL |
| x02316-02 | ALDLNTSAGF PYIAMGVKKR DLINNKTKDI SKLKEAIDKY GVDLPMVTFL |
| hrv-07 | ALDLNTSAGF PYVSLGIKKK DLIDKKARDI SKLRKAIDKY GIDLPMVTFL |
| dq473503-07 | ALDLNTSAGF PYVSLGIKKK DLIDKKARDI SKLRKAIDKY GIDLPMVTFL |
| hrv-08 | ALDLNTSAGF PYVALGIKKK DIINKSTRDT SKIKNCLDTY GVDLPMITYL |
| pico-09 | ALDLNTSAGF PYVSMGIKKR DLINNNTKDI SKLRAALDKY GVDLPMVTFL |
| hrv-09 | ALDLNTSAGF PYVSMGIRKR DLINNSTKDI SKLRAALDKY GVDLPMVTFL |
| hrv-09-f01 | ALDLNTSAGF PYVSMGIKKR DLINNSTKDI SKLRAALDKY GVDLPMVTFL |
| hrv-09-f02 | ALDLNTSAGF PYVSMGIKKR DLINNSTKDI SKLRAALDKY GVDLPMVTFL |
| dq473498-10 | ALDLNTSAGF PYVSMGIKKK DLINKQTKDV TKLKMALDKY GVDLPMVTFL |
| hrv-10 | ALDLNTSAGF PYVSMGIKKK DLINKQTKDV TKLKMALDKY GVDLPMVTFL |
| ef173414-11 | ALDLNTSAGY PYVTMGIKKK DLIDNRNKDL TKLKVALDKY GVDLPMVTFL |
| ef173415-12 | ALDLNTSAGY PYTSLGIKKR DLIGRD.KDL SKLKAAINKY GLDLPMITFL |
| hrv-13 | ALDLNTSAGF PYISMGIKKR DLINNSTKDI TKLKLALDKY GVDLPMVTFL |
| hrv-13-f03 | ALDLNTSAGF PYISMGIKKR DLINNTTKDI TKLKLALDKY GVDLPMVTFL |
| dq473493-15 | ALDLNTSAGF PYISLGIKKR DLINNNTKDL SRLKSALDKY GVDLPMVTFL |
| l24917-16 | ALDLNTSAGY PYVTLGIKKK DLINNKTKDI SKLKLALDKY DVDLPMITFL |
| hrv-18 | ALDLNTSAGF PYVSMGIRKR DLINKQTKDI TKLKLALDKY GVDLPMVTFL |
| hrv-19 | ALDLNTSAGF PYISMGIKKR DLINNSTKDI SKLKMALDKY GVDLPMVTFL |
| hrv-20 | ALDLHTSAGY PYTACGIKKK DLIPKD.KNL TKLKHAIEKY GLDLPMITFL |
| hrv-21 | ALDLNTSAGF PYVTMGIKKR DLINKQTKDI TKLKMALDKY GVDLPMVTFL |
| hrv-22 | ALDLNTSAGF PYISMGIKKR DLINNSTKDI SKLKLALDKY GVDLPMVTFL |
| dq473497-23 | ALDLNTSAGF PYITMGIKKR DLINNKTKDI SKLREALDKY GVDLPMVTFL |
| hrv-24 | ALDLNTSAGF PYVTMGIKKR DLINKQTRDI TKLKMAMDKY GVDLPMVTFL |
| ef173416-24 | ALDLNTSAGF PYVTMGIKKR DLINKQTRDI TKLKMAMDKY GVDLPMVTFL |
| hrv-25 | ALDLNTSAGF PYVSMGIKKR DLINKQTKDL TKLKVALDKY GVDLPMVTFL |
| dq473508-28 | ALDLHTSAGY PYTACGIKKK DLIPKD.RNL TKLKCAIEKY GLDLPMITFL |
| hrv-29 | ALDLNTSAGF PYISMGIKKR DLINKQTKDV TKLKMALDKY GVDLPMVTFL |
| dq473512-30 | ALDLNTSAGF PYITMGIKKR DLINNRTKDI SKLKEAIDKY GVDLPMVTFL |
| hrv-30 | ALDLNTSAGF PYITMGIKKR DLINNRTKDI SKLKEAIDKY GVDLPMVTFL |
| hrv-31 | ALDLTTSAGF PYVSMGIKKR DLINKQTKDI TKLKMALDKY GVDLPMVTFL |
| hrv-32 | ALDLNTSAGF PYISMGIKKR DLINNSTKDI SKLKIALDKY GVDLPMVTFL |
| hrv-33 | ALDLNTSAGY PYVTMGIKKK DLIDNRAKDL TKLKIAMDKY GVDLPMVTFL |
| hrv-34 | ALDLNTSAGF PYVSMGIKKR DLINKQTKDI SKLKLALDKY GVDLPMVTFL |
| dq473501-34 | ALDLNTSAGF PYVSMGIKKR DLINKQTKDI SKLKLALDKY GVDLPMVTFL |
| dq473505-36 | ALDLNTSAGF PYVSLGIKKK DLIDKKTKDI TKLKKAIDKY GIDLPMVTFL |
| hrv-38 | ALDLNTSAGF PYVSMGVKKR DLINKQTKDV TRLKAAMDKY GVDLPMVTFL |
| dq473495-38 | ALDLNTSAGF PYVSMGVKKR DLINKQTKDV TRLKAAMDKY GVDLPMVTFL |
| ay751783-39 | ALDLNTSAGF PYITMGIKKR DLINNKTKDI SRLKQAIDKY GVDLPMVTFL |
| hrv-40 | ALDLNTSAGF PYVSMGIKKR DLINKQTKDI TKLKAALDKY GVDLPMVTFL |
| dq473491-41 | ALDLNTSAGF PYISMGIKKR DLINNSTKDI TKLKIALDKY GVDLPMVTFL |
| hrv-43 | ALDLNTSAGF PYISMGIKKR DLINKNTRDI SKLKAALDKY GVDLPMVTFL |
| dq473499-44 | ALDLNTSAGF PYISMGIKKR DLINKQTKDV TKLKMALDKY GVDLPMVTFL |
| hrv-45 | ALDLNTSAGF PYVALGIKKK DLINRETRDV SKIKNCLDTY GVDLPMITYL |
| dq473506-46 | ALDLHTSAGY PYTAHGIKKK DLIPKD.RNL TKLKVAMEKY GLDLPMITFL |
| hrv-47 | ALDLTTSAGF PYVSMGIKKR DLINKQTRDI TKLKMALDKY GVDLPMVTFL |
| dq473496-49 | ALDLNTSAGF PYITMGIKKR DLINNKTKDI SKLREAIDKY GVDLPMVTFL |
| hrv-49-f04 | ALDLNTSAGF PYITMGIKKR DLINNKTKDI SKLREAIDKY GVDLPMVTFL |
| hrv-50 | ALDLNTSAGF PYVSMGIKKR DLINKQTKDI SKLKMALDKY GVDLPMVTFL |
| hrv-51 | ALDLHTSAGY PYTACGIKKK DLIPKD.RDL TKFKQALEKY GLDLPMITFL |
| dq473507-53 | ALDLHTSAGY PYTAHGIKKK DLIPKD.KNL TKLKIAMEKY GLDLPMITFL |
| hrv-54 | ALDLTTSAGF PYVSMGIKKR DLINKQTKDI TKLKMALDKY GVDLPMVTFL |
| hrv-54-f05 | ALDLTTSAGF PYVSMGIKKR DLINKQTKDI TKLKIALDKY GVDLPMVTFL |
| dq473511-55 | ALDLNTSAGF PYVTMGIKKR DLINKQTKDI SKLKIALDKY GVDLPMVTFL |
| hrv-56 | ALDLNTSAGF PYVSMGIKKK DLINKQTKDL TKLKIALDKY GVDLPMVTFL |
| hrv-57 | ALDLNTSAGF PYVAMGIKKR DLINKQTRDI TKLKMALDKY GVDLPMVTFL |
| hrv-58 | ALDLNTSAGF PYVSLGIKKK DLIDKKTKDI TKLKKAIDKY GIDLPMVTFL |
| dq473500-59 | ALDLNTSAGF PYVAMGIKKK DLIDNRTKDI SKLKVALDKY GVDLPMVTFL |
| hrv-60 | ALDLNTSAGF PYVSMGIKKR DLINKQTKDI TKLKMAMDKY GVDLPMVTFL |
| hrv-61 | ALDLNTSAGF PYISMGIRKR DLINNSTKDI TKLKVALDKY GVDLPMVTFL |
| hrv-62 | ALDLNTSAGF PYVSMGIKKR DLINKQTRDL TKLKVALDKY GVDLPMVTFL |
| hrv-63 | ALDLNTSAGF PYVSMGIKKR DLINKQTKDI TKLKVALDKY GVDLPMVTFL |
| hrv-64 | ALDLNTSAGF PYISMGIKKR DLINNSTKDI SKLKLALDKY GVDLPMVTFL |
| ef173417-64 | ALDLNTSAGF PYISMGIKKR DLINNSTKDI SKLKLALDKY GVDLPMVTFL |
| hrv-65 | ALDLHTSAGY PYTACGIKKK DLIPKD.KNL TKLKQAMEKY GLDLPMITFL |
| hrv-66 | ALDLNTSAGF PYVTMGVKKR DLINKQTKDV TKLKAAMDKY GVDLPMVTFL |
| hrv-67 | ALDLNTSAGF PYISMGIKKR DLINNSTKDI SKLKIALDKY GVDLPMVTFL |
| hrv-68 | ALDLHTSAGY PYTACGIKKK DLIPKD.RNL TRLKCAIEKY GLDLPMITFL |
| hrv-71 | ALDLHTSAGY PYTACGIKKK DLIPKN.RDL SKLKGAIEKY GLDLPMITFL |
| dq473492-73 | ALDLNTSAGF PYISMGIRKK DLINNSTKDI TKLKLALDKY GVDLPMVTFL |
| dq473494-74 | ALDLNTSAGF PYISMGIKKR DLINNSTKDI SKLKTALDKY GVDLPMVTFL |
| dq473510-75 | ALDLNTSAGF PYISMGIKKR DLINNTTKDL SKLKAAMDKY GVDLPMVTFL |
| dq473502-76 | ALDLNTSAGY PYVTMGIKKK DLIDNRNKDL TKLKIAMDKY GVDLPMVTFL |
| hrv-76 | ALDLNTSAGY PYVTMGIKKK DLIDNRNKDL TKLKIAMDKY GVDLPMVTFL |
| hrv-77 | ALDLNTSAGF PYVSMGIKKR DLINKQTKDI TKLKVALDKY GVDLPMVTFL |
| hrv-78 | ALDLHTSAGY PYTTMGIKKK DLINTKTKDV TKLKEVMNKY GVDLPMITFL |
| ef173418-78 | ALDLHTSAGY PYTTMGIKKK DLINTKTKDV TKLKEVMNKY GVDLPMITFL |
| hrv-80 | ALDLHTSAGY PYTANGIKKK DLIPKD.RDL TKLKLAMEKY GLDLPMITFL |
| hrv-81 | ALDLNTSAGY PYVALGIKKK DLINNKTKDI SKLKVALDKY GVDLPMITFL |
| hrv-81-f06 | ALDLNTSAGY PYVSLGIKKR DLINNKTKDI SKLKVALDKY GVDLPMITFL |
| hrv-81-f07 | ALDLNTSAGY PYVSLGIKKR DLINNKTKDI SKLKVALDKY GVDLPMITFL |
| hrv-82 | ALDLNTSAGF PYISMGIKKR DLIDKSTKDI SKLKVALDKY GVDLPMVTFP |
| dq473509-82 | ALDLNTSAGF PYISMGIKKR DLIDKNTKDI SKLKIALDKY GVDLPMVTFL |
| hrv-85 | ALDLNTSAGF PYVSMGIRKR DLINKQTKDI TKLKMALDKY GVDLPMVTFL |
| pico-85 | ALDLNTSAGF PYVSMGIRKR DLINKQTKDI TKLKMALDKY GVDLPMVTFL |
| dq473504-88 | ALDLSTSAGF PYVSLGIKKK DLIDKKTKDI TKLKKAIDEY GLDLPMVTFL |
| a10937-89 | ALDLNTSAGF PYVSLGIKKK DLIDKKTKDI TKLRKAIDEY GIDLPMVTFL |
| hrv-89 | ALDLNTSAGF PYVSLGIKKK DLIDKKTKDI TKLRKAIDEY GIDLPMVTFL |
| hrv-89-f08 | ALDLNTSAGF PYVSLGIKKK DLIDKKTRDI TKLKKAIDEY GIDLPMVTFL |
| hrv-89-f09 | ALDLNTSAGF PYVSLGIKKK DLIDKRTRDI TKLKKAIDEY GIDLPMVTFL |
| m16248-89 | ALDLNTSAGF PYVSLGIKKK DLIDKKTKDI TKLRKAIDEY GIDLPMVTFL |
| hrv-90 | ALDLNTSAGF PYVTMGIKKR DLINKQTKDI TKLKMAMDKY GVDLPMVTFL |
| hrv-94 | ALDLNTSAGF PYISMGIKKR DLINNSTKDI SKLKIALDKY GVDLPMVTFL |
| ef173419-94 | ALDLNTSAGF PYISMGIKKR DLINNSTKDI SKLKIALDKY GVDLPMVTFL |
| hrv-95 | ALDLNTSAGF PYVALGIKKK DIINKSTRDT SKIKNCLDTY GVDLPMITYL |
| hrv-96 | ALDLNTSAGF PYISMGIRKR DLINNNTKDI TKLKIALDKY GVDLPMVTFL |
| hrv-98 | ALDLTTSAGF PYVSMGIKKR DLINKQTKDI TKLKIALDKY GVDLPMVTFL |
| hrv-100 | ALDLNTSAGF PYVSMGIKKR DLINKQTKDV TKLKMALDKY GVDLPMVTFL |
| ef186077-qpm | PLDLTTSAGF PYVTMGVKKR DILNRNTRDV TKLQHMIDTY GLDLPYVTYL |
| ef077279-nat001 | PLDLTTSAGF PYVTMGVKKR DILNRNTKDV TKLQHMIDTY GLDLPYITYL |
| ny1078 | PLDLTTSAGF PYVTMGVKKR DILNRITKDT TKLQEMIDTY GLDLPYITYL |
| ef582385-c024 | PLDLTTSPGF PYVSLGVKKK DILNNATKDT RKMQQMLDLY GIDLPYITFL |
| ef077280-nat045 | ALDLTTSAGF PYVSKGIKKK DILNKTTRDT TKLAKLMEEY GIDLPYVTYL |
| ef582387-c026 | PLDLTTSAGF PYVTMGIKKR DILNRNTKDI TKLQQMIDTY GLDLPYVTYL |
| ef582386-c025 | PLDLNTSAGY PYVTLGVKKR DILNRNTKDL SKLQHMMDLY GIDLPYITYL |
| dq473485-03 | PIDVTTSAGY PYVSLGIKKR DILNKETQDV EKMKFYLDKY GIDLPLVTYI |
| ef173422-03 | PIDVTTSAGY PYVSLGIKKR DILNKETQDV EKMKFYLDKY GIDLPLVTYI |
| dq473490-04 | PIDLNTSAGY PYVSLGINKK SILNRETKEV AKMQECLSKY GIDLPLITYI |
| hrv-05 | PIDLNTSAGY PYVSLGIKKR SILNKETKDT SKMEECLSKY GIDLPLVTYI |
| dq473486-06 | PIDITTSAGY PYVSLGIKKR DILNRETQEV EKMKFYLDKY GIDLPLVTYI |
| l05355-14 | PIDITTSAGF PYVSLGIKKR DILNKETQDT EKMKFYLDKY GIDLPLVTYI |
| k02121-14 | PIDITTSAGF PYVSLGIKKR DILNKETQDT EKMKFYLDKY GIDLPLVTYI |
| ef173420-17 | PIDINTSAGY PYVSLGIKKR DILNRDTQDI AKMEHYLDKY GIDLPLVTYI |
| hrv-26 | PIDLNTSAGY PYVSLGIKKR AILNKDTKDT SKMEECLSKY GIDLPLVTYI |
| hrv-27 | PIDLNTSAGY PYVSLGIKKR AILNKETQDT TKMEECLSKY GIDLPLVTYI |
| ef173421-27 | PIDLNTSAGY PYVSLGIKKR AILNKETQDT TKMEECLSKY GIDLPLVTYI |
| dq473487-35 | PIDVTTSAGY PYVSLGIKKR DILNRETQEV EKMKFYLDKY GIDLPLVTYI |
| hrv-35 | PIDVTTSAGY PYVSLGIKKR DILNRETQEV EKMKFYLDKY GIDLPLVTYI |
| ef173423-37 | PIDVTTSAGY PYVSLGIKKR DILNRETQET EKMKFYLDKY GIDLPLVTYI |
| hrv-42 | PIDLNTSAGY PYVSLGIKKR SILNKETKDT TKMEECLSKY GIDLPLVTYI |
| dq473488-48 | PIDVNTSAGY PYVSLGIRKR DILNKETQDT IKMEHYLDKY GIDLPLVTYI |
| hrv-52 | PIDINTSAGY PYVSLGIKKK DILNRETQDT SKMEHYLDKY GIDLPLVTYI |
| hrv-52-f10 | PIDINTSAGY PYVSLGIKKK DILNRETQDT SKMEHYLDKY GIDLPLVTYI |
| ef173424-52 | PIDINXSAGY PYVSLGIKKK DILNRETQDT SKMEHYLDKY GIDLPLVTYI |
| hrv-69 | PIDINTSAGY PYVSLGIKKK DILNRETQDT SKMEHYLDKY GIDLPLVTYI |
| dq473489-70 | PIDINTSAGY PYVSLGIKKR DILNRDTQDV TKMEHYLDKY GIDLPLVTYI |
| hrv-72 | PIDVTTSAGY PYVSLGIKKR DIINKETKDV EKMKYYLDKY GIDLPLVTYI |
| hrv-79 | PIDVTTSAGY PYVSLGIKKR DILNKETQDT EKMKFYLDKY GIDLPLVTYI |
| hrv-83 | PIDITTSAGY PYVSLGIKKR DILNKETQEV EKMKFYLDKY GIDLPLVTYI |
| hrv-84 | PIDLNTSAGY PYVGLGIKKR SILNKETQDT TKMQECLSKY GIDLPLITYI |
| hrv-86 | PLDITTSAGY PYVSLGIKKR DILNRETQDX TKMQAYLDKY GIDLPLVTYI |
| hrv-91 | PIDINTSAGY PYVSLGIKKR DILNKDTQDV AKMEHYLDKY GIDLPLVTYI |
| hrv-92 | PIDITTSAGY PYVSLGIKKR DILNKETQET EKMKFYLDKY GIDLPLVTYI |
| ef173425-93 | PIDLNTSAGY PYVSLGIKKR SILNKETQDT TKMEECLDKY GIDLPLVTYI |
| hrv-97 | PIDLNTSAGY PYVSLGIKKR SILNKETKDT AKMEECLSKY GIDLPLVTYI |
| hrv-99 | PIDLNTSAGY PYVNLGIKKR SILNKETKDT TKMEECLSKY GIDLPLVTYI |
| | 1951 2000 |
| annotation | .....HHHHH HH....BBBB .HHHHHHHHH HHHHHHHHHH HH........ |
| af499637-cva13 | KDELRSKTKV EQGKSRLIEA SSLNDSVAMR MAFGNLYAAF HKNPGVVTGS |
| af546702-cva21 | KDELRSKTKV EQGKSRLIEA SSLNDSVAMR MAFGNLYAAF HKNPGVVTGS |
| v01149-pv1m | KDELRSKTKV EQGKSRLIEA SSLNDSVAMR MAFGNLYAAF HKNPGVITGS |
| d00239-01 | KDELRKKEKI SAGKTRVIEA SSINDTILFR TTFGNLFSKF HLNPGVVTGS |
| pico-01 | KDELRKKEKI STGKTRVIEA SSINDTILFR TTFGNLFSKF HLNPGVVTGS |
| hrv-01 | KDELRKKEKI STGKTRVIEA SSINDTILFR TTFGNLFSKF HLNPGVVTGS |
| x02316-02 | KDELRKHEKV IKGKTRVIEA SSVNDTLLFR TTFGNLFSKF HLNPGIVTGS |
| hrv-07 | KDELRRKEKI KEGKTRVIEA NSVNDTVVFR STFGNLFSTF HKNPGIVTGS |
| dq473503-07 | KDELRRKEKI KEGKTRVIEA NSVNDTVVFR STFGNLFSTF HKNPGIVTGS |
| hrv-08 | KDELRAPEKV KMGKTRVIEA SSLNDTVHMR MLFGNLFKAF HANPGIITGC |
| pico-09 | KDELRKKEKI SSGKTRVIEA SSINDTVTFR MTYGNLFACF HKNPGIVTGS |
| hrv-09 | KDELRKKEKI SSGKTRVIEA SSINDTVAFR MTYGNLFACF HKNPGIITGS |
| hrv-09-f01 | KDELRKKEKI SSGKTRVIEA SSVNDTVAFR MTYGNLFACF HKNPGIVTGS |
| hrv-09-f02 | KDELRKKEKI SSGKTRVIEA SSVNDTVAFR MTYGNLFACF HKNPGIVTGS |
| dq473498-10 | KDELRKREKI TAGKTRVIEA SSVNDTVLFR TTFGNLFSKF HLNPGIVTGS |
| hrv-10 | KDELRKREKI TAGKTRVIEA SSVNDTVLFR TTFGNLFSKF HLNPGIVTGS |
| ef173414-11 | KDELRKKEKI SAGKTRVIEA SSVNDTVLFR SIYGNLFSKF HLNPGVVTGS |
| ef173415-12 | KDELRKPEKI KAGKTRVIEA SSINDTIHFR MKFGNLFSSF HLNPGIVTGS |
| hrv-13 | KDELRKKEKI STGKTRVIEA SSVNDTIAFR VVYGNLFSTF HKNPGVVTGS |
| hrv-13-f03 | KDELRKKEKI STGKTRVIEA SSVNDTIAFR VVYGNLFSSF HKNPGVVTGS |
| dq473493-15 | KDELRKKEKI SAGKTRVIEA SSVNDTVTFR MVYGNLFSTF HKNPGIITGS |
| l24917-16 | KDELRKKDKI AAGKTRVIEA SSINDTILFR TVYGNLFSKF HLNPGVVTGC |
| hrv-18 | KDELRKKEKI SAGKTRVIEA SSVNDTVLFR TTFGNLFSKF HLNPGIVTGS |
| hrv-19 | KDELRKKEKI SAGKTRVIEA SSINDTVAFR IVYGKLFAAF HKNPGVVTGS |
| hrv-20 | KDELRKPEKI KTGKTRIIEA SSLNDTVQFR MAFGNLFSKF HQNPGIITGS |
| hrv-21 | KDELRKKEKI SAGKTRVIEA SSVNDTVLFR TTFGNLFSKF HLNPGIVTGS |
| hrv-22 | KDELRKKEKI AAGKTRVIEA SSVNDTVAFR MVYGNLFSAF HKNPGIVTGS |
| dq473497-23 | KDELRKQEKI IKGKTRVIEA SSVNDTLLFR TTFGNLFSKF HLNPGIVTGS |
| hrv-24 | KDELRKREKI SAGKTRVIEA SSVNDTVLFR TTFGNLFSKF HLNPGIVTGS |
| ef173416-24 | KDELRKREKI SAGKTRVIEA SSVNDTVLFR TTFGNLFSKF HLNPGIVTGS |
| hrv-25 | KDELRKKEKI RAGKTRVIEA SSVNDTILFR TTFGNLFSKF HLNPGIITGS |
| dq473508-28 | KDELRKPEKI KTGKTRIIEA SSLNDTVQFR MAFGNLFSKF HQNPGIVTGS |
| hrv-29 | KDELRKREKI CAGKTRVIEA SSVNDTILFR TTFGNLFSKF HLNPGVVTGS |
| dq473512-30 | KDELRKPEKI SKGKTRVIEA SSVNDTLLFR TTFGNLFSKF HLNPGIVTGS |
| hrv-30 | KDELRKPEKI SKGKTRVIEA SSVNDTLLFR TTFGNLFSKF HLNPGIVTGS |
| hrv-31 | KDELRKKEKI ATGKTRVIEA SSVNDTILFR TTFGNLFSKF HLNPGIVTGS |
| hrv-32 | KDELRKKEKI PSGKTRVIEA SSVNDTIAFR MTYGNLFACF HKNPGVVTGS |
| hrv-33 | KDELRKKEKI SAGKTRVIEA SSVNDTVLFR SVYGNLFSKF HLNPGIITGS |
| hrv-34 | KDELRKKEKI SAGKTRVIEA SSVNDTVLFR TTFGNLFSKF HLNPGIVTGS |
| dq473501-34 | KDELRKKEKI SAGKTRVIEA SSVNDTVLFR TTFGNLFSKF HLNPGIVTGS |
| dq473505-36 | KDELRKKEKI RDGKTRVIEA NSVNDTVLFR SVYGNLFSAF HRNPGIVTGS |
| hrv-38 | KDELRKQEKI SAGKTRVIEA SSVNDTVMFR TTFGNLFSKF HLNPGIVTGS |
| dq473495-38 | KDELRKQEKI SAGKTRVIEA SSVSDTVMFR TNFGNLFSKF HLNPGIVTGS |
| ay751783-39 | KDELRKEEKI AKGKTRVIEA SSVNDTLLFR TTFGNLFSKF HLNPGIVTGS |
| hrv-40 | KDELRKKEKI SAGKTRVIEA SSVNDTVLFR ATFGNLFSKF HLNPGIVTGS |
| dq473491-41 | KDELRKKEKI AAGKTRVIEA SSVNDTIAFR VTYGNLFSTF HRNPGIITGS |
| hrv-43 | KDELRKKEKI SAGKTRVIEA SSVNDTVAFR MIYGNLFAAF HKNPGIVTGS |
| dq473499-44 | KDELRKREKI CDGKTRVIEA SSVNDTILFR TTFGNLFSKF HLNPGVVTGS |
| hrv-45 | KDELRTPEKI KLGKTRVIEA SSLNDTIHMR MLFGNLFKAF HANPGIVTGC |
| dq473506-46 | KDELRKPEKI SAGKTRIIEA SSLNDTVQFR MAFGNLFSKF HKNPGIVTGS |
| hrv-47 | KDELRKKEKI SAGKTRVIEA SSVNDTVLFR TTFGNLFSKF HLNPGIVTGS |
| dq473496-49 | KDELRKHEKI IKGKTRVIEA SSVNDTLLFR TTFGNLFSKF HLNPGIITGS |
| hrv-49-f04 | KDELRKHEKI IKGKTRVIEA SSVNDTLLFR TTFGNLFSKF HLNPGIVTGS |
| hrv-50 | KDELRKKEKI SAGKTRVIEA SSVNDTVLFR TTFGNLFSKF HLNPGIVTGS |
| hrv-51 | KDELRKPEKI LAGKTRIIEA SSLNDTVQFR MAFGRLFSAF HQNPGIVTGS |
| dq473507-53 | KDELRKPEKI SAGKTRIIEA SSLNDTVQFR MAFGNLFSKF HKNPGIVTGS |
| hrv-54 | KDELRKKEKI SAGKTRVIEA SSVNDTVLFR TTFGNLFSKF HLNPGIVTGS |
| hrv-54-f05 | KDELRKKEKI HTGKTRVIEA SSVNDTVLFR TTFGNLFSKF HLNPGIVTGS |
| dq473511-55 | KDELRKKDKI SAGKTRVIEA SSVNDTVLFR TTFGNLFSKF HLNPGVVTGS |
| hrv-56 | KDELRKKEKI AAGKTRVIEA SSVNDTILFR TTFGNLFSKF HLNPGIVTGS |
| hrv-57 | KDELRKKEKI SAGKTRVIEA SSVNDTVLFR TTFGNLFSKF HLNPGIVTGS |
| hrv-58 | KDELRKKEKI RDGKTRVIEA NSVNDTVLFR SVYGNLFSTF HKNPGIVTGS |
| dq473500-59 | KDELRKKDKI RAGKTRVIEA SSINDTLLFR TTFGNLFSKF HLNPGIVTGS |
| hrv-60 | KDELRKQEKI SAGKTRVIEA SSVNDTVLFR TTFGNLFSKF HLNPGIVTGS |
| hrv-61 | KDELRKKGKI MAGKTRVIEA SSINDTVAFR MTYGKLFSSF HKNPGIITGS |
| hrv-62 | KDELRKKEKI CAGKTRVIEA SSVNDTILFR TTFGNLFSKF HLNPGIITGS |
| hrv-63 | KDELRKKEKI SAGKTRVIEA SSVNDTVLFR TTFGNLFSKF HLNPGIVTGS |
| hrv-64 | KDELRKKEKI SAGKTRVIEA SSVNDTVAFR MVYGNLFAAF HKNPGIITGS |
| ef173417-64 | KDELRKKEKI SAGKTRVIEA SSVNDTVAFR MVYGNLFAAF HKNPGIITGS |
| hrv-65 | KDELRKPDKI YTGKTRIIEA SSLNDTVQFR MAFGRLFSSF HQNPGIITGS |
| hrv-66 | KDELRKKEKI SAGKTRVIEA SSVNDTILFR MTFGNLFSKF HLNPGVVTGS |
| hrv-67 | KDELRKKEKI SSGKTRVIEA SSINDTITFR MTYGNLFSCF HKNPGVVTGS |
| hrv-68 | KDELRKPEKI KTGKTRIIEA SSLNDTIQFR MAFGNLFSKF HQNPGIVTGS |
| hrv-71 | KDELRKPEKI NTGKTRIVEA SSLNDTVQFR MAFGRLFSVF HQNPGIITGS |
| dq473492-73 | KDELRKKEKI STGKTRVIEA SSVNDTIAFR VVYGNLFSTF HKNPGVVTGS |
| dq473494-74 | KDELRKKEKI SAGKTRVIEA SSINDTVTFR MVYGNLFTNF HKNPGVVTGS |
| dq473510-75 | KDELRKKEKI SAGKTRVIEA SSINDTIIFR QTYGNLFAAF HKNPGIITGS |
| dq473502-76 | KDELRKKEKI SAGKTRVIEA SSVNDTVLFR SVYGNLFSKF HLNPGIITGS |
| hrv-76 | KDELRKKEKI SAGKTRVIEA SSVNDTVLFR SVYGNLFSKF HLNPGIITGS |
| hrv-77 | KDELRKKEKI SAGKTRVIEA SSINDTVLFR STFGNLFSKF HLNPGIITGS |
| hrv-78 | KDELRKKDKI VAGKTRVIEA SSINDTVQFR MTFGRLFQAF HNNPGIVTGS |
| ef173418-78 | KDELRKKDKI VAGKTRVIEA SSINDTVQFR MTFGRLFQAF HNNPGIVTGS |
| hrv-80 | KDELRKPEKI REGKTRIIEA SSLNDTVHFR VTFGNLFSKF HQNPGIVTGS |
| hrv-81 | KDELRKKDKI PVGKTRVIEA SSINDTVLFR TTYGNLFSKF HLNPGVVTGC |
| hrv-81-f06 | KDELRKKDKI SAGKTRVIEA SSINDTILFR TTYGNLFSKF HLNPGVVTGC |
| hrv-81-f07 | KDELRKKDKI SAGKTRVIEA SSINDTILFR TTYGNLFSKF HLNPGVVTGC |
| hrv-82 | KDELRKKEKI SSGKTRVIEA SSVNDTVAFR MVYGNLFAAF HKNPGIVTGS |
| dq473509-82 | KDELRKKEKI SSGKTRVIEA SSVNDTVAFR MVYGNLFAAF HKNPGIVTGS |
| hrv-85 | KDELRKKEKI SAGKTRVIEA SSVNDTVLFR TTFGNLFSKF HLNPGIVTGS |
| pico-85 | KDELRKKEKI SAGKTRVIEA SSVNDTVLFR TTFGNLFSKF HLNPGIVTGS |
| dq473504-88 | KDELRRKEKI KDGKTRVIEA NSVNDTVIFR SVFGNLFSAF HKNPGVVTGS |
| a10937-89 | KDELRKKEKI KDGKTRVIEA NSVNDTVLFR SVFGNLFSAF HKNPGIVTGS |
| hrv-89 | KDELRKKEKI KDGKTRVIEA NSVNDTVLFR SVFGNLFSAF HKNPGIVTGS |
| hrv-89-f08 | KDELRKKEKI KDGKTRVIEA NSVNDTVLFR SVFGNLFSAF HKNPGIVTGS |
| hrv-89-f09 | KDELRKKEKI KDGKTRVIEA NSVNDTVLFR SVFGNLFSAF HKNPGIVTGS |
| m16248-89 | KDELRKKEKI KDGKTRVIEA NSVNDTVLFR SVFGNLFSAF HKNPGIVTGS |
| hrv-90 | KDELRKKEKI SAGKTRVIEA SSVNDTVLFR TTFGNLFSKF HLNPGVVTGS |
| hrv-94 | KDELRKKXKI STGKTRVIEA SSVNDTVAFR MVYGNLFSAF HRNPGIVTGS |
| ef173419-94 | KDELRKKGKI STGKTRVIEA SSVNDTVAFR MVYGNLFSAF HRNPGIVTGS |
| hrv-95 | KDELRAPEKV KMGKTRVIEA SSLNDTVHMR MLFGNLFKAF HANPGIITGC |
| hrv-96 | KDELRKKEKI SAGKTRVIEA SSINDTVAFR MVYGRLFSTF HKNPGIITGS |
| hrv-98 | KDELRKREKI SAGKTRVIEA SSVNDTVLFR TTFGNLFSKF HLNPGIVTGS |
| hrv-100 | KDELRKKEKI RAGKTRVIEA SSVNDTVLFR TTFGNLFSKF HLNPGIVTGS |
| ef186077-qpm | KDELRAPAKV KAGKTRAIEA ASMNDTANFR RVFGNLYAGF HANPGILTGS |
| ef077279-nat001 | KDELRAPAKI KAGKTRAIEA ASMNDTANFR RVFGNLYAGF HANPGILTGS |
| ny1078 | KDELRSPAKI KAGKTRAIEA ASMNDTANFR RVFGNLYASF HANPGVLTGS |
| ef582385-c024 | KDELRAPEKI KAGKTRIVEA ASVNDTVYFR TTFGNLYSTF HANPGILTGS |
| ef077280-nat045 | KDELRTPEKI KAGKTRLIEA ASLNDTVHYR MVFGNLFSTL HANPGILTGS |
| ef582387-c026 | KDELRAPTKV KAGKTRAIEA ASMNDTANFR RVFGNLYAGF HANPGILTGS |
| ef582386-c025 | KDELRAPEKI RCGKTRCIEA ASMNDTVYFR MVFGNLYGAF HKNPGVLTGS |
| dq473485-03 | KDELRSVDKV RLGKSRLIEA SSLNDSVNMR MKLGNLYKAF HQNPGIITES |
| ef173422-03 | KDELRSVDKV RLGKSRLIEA SSLNDSVNMR MKLGNLYKAF HQNPGIITGS |
| dq473490-04 | KDELRGKDKV RLGKSRLIEA SSLNDSVNMR VKLGNLYKAF HKNPGVLTGS |
| hrv-05 | KDELRGKDKI RLGKSRLIEA SSLNDSVNMR VKLGNLYKAF HKNPGILTGS |
| dq473486-06 | KDELRSTDKV RLGKSRLIEA SSLNDSVNMR MKLGNLYKAF HQNPGIITGS |
| l05355-14 | KDELRSVDKV RLGKSRLIEA SSLNDSVNMR MKLGNLYKAF HQNPGVLTGS |
| k02121-14 | KDELRSVDKV RLGKSRLIEA SSLNDSVNMR MKLGNLYKAF HQNPGVLTGS |
| ef173420-17 | KDELRSKDKV RLGKSRLIEA SSLNDSVNMR VKLGNLYKAF HTNPGVITGS |
| hrv-26 | KDELRGKDKI RLGKSRLIEA SSLNDSVNMR VKLGNLYKAF HKNPGILTGS |
| hrv-27 | KDELRGRDKV RLGKSRLIEA SSLNDSVNMR VKLGELYKAF HKNPGILTGS |
| ef173421-27 | KDELRGRDKV RLGKSRLIEA SSLNDSVNMR VKLGELYKAF HKNPGILTGS |
| dq473487-35 | KDELRSVDKV RLGKSRLIEA SSLNDSVNMR MKLGNLYKAF HKNPGTITGS |
| hrv-35 | KDELRSVDKV RLGKSRLIEA SSLNDSVNMR MKLGNLYKAF HKNPGTITGS |
| ef173423-37 | KDELRSTDKV RLGKSRLIEA SSLNDSVNMR MKLGNLYKAF HQNPGVITGS |
| hrv-42 | KDELRGKDKI RLGKSRLIEA SSLNDSVNMR IKLGNLYKAF HKNPGILTGS |
| dq473488-48 | KDELRGKDKV RLGKSRLIEA SSLNDSVNMR VKLGNLYKAF HLNPGVITGS |
| hrv-52 | KDELRGRDKV RLGKSRLIEA SSLNDSVNMR VKLGNLYKAF HSNPGVITGS |
| hrv-52-f10 | KDELRGKDKV RLGKSRLIEA SSLNDSVNMR VKLGNLYKAF HLNPGVITGS |
| ef173424-52 | KDELRGRDKV RLGKSRLIEA SSLNDSVNMR VKLGNLYKAF HSNPGVITGS |
| hrv-69 | KDELRGKDKV RLGKSRLIEA SSLNDSVNMR VKLGNLYKAF HLNPGVITGS |
| dq473489-70 | KDELRSKDKV RLGKSRLIEA SSLNDSVNMR VKLGNLYKAF HTNPGVITGS |
| hrv-72 | KDELRSTDKV RLGKSRLIEA SSLNDSVNMR MKLGNLYKAF HQNPGTITGS |
| hrv-79 | KDELRSSDKV RLGKSRLIEA SSLNDSVNMR MKLGHLYKAF HQNPGVITGS |
| hrv-83 | KDELRSADKV RLGKSRLIEA SSLNDSVNMR MKLGHLYKTF HQNPGVITGS |
| hrv-84 | KDELRGKVKI RLGKSRLIEA SSLNDSVNMR VKLGHLYKAF HKNPGTLTGS |
| hrv-86 | KDELRSVDKV RLGKSRLIEA SSLNDSVNMR MKLGSLYKTF HQNPGIITGS |
| hrv-91 | KDELRSKDKI RLGKSRLIEA SSLNDSVNMR VKLGNLYKAF HTNPGVITGS |
| hrv-92 | KDELRSADKV RLGKSRLIEA SSLNDSVNMR MKLGHLYKTF HQNPGVITGS |
| ef173425-93 | KDELRGKDKV RLGKSRLIEA SSLNDSVNMR VKLGELYKAF HKNPGIMTGS |
| hrv-97 | KDELRGKDKV RLGKSRLIEA SSLNDSVNMR VKLGELYKAF HKNPGILTGS |
| hrv-99 | KDELRGKDKI RLGKSRLIEA SSLNDSVNMR VKLGNLYKAF HLNPGILTGS |
| | 2001 2050 |
| annotation | ....HHHHHH .HHHHHH..B .BB.BB..HH HHHHHHHHHH HHHHHHHHH. |
| af499637-cva13 | AVGCDPDLFW SKIPVLMEEK .LFAFDYTGY DASLSPAWFE ALKMVLEKIG |
| af546702-cva21 | AVGCDPDLFW SKIPVLMEEK .LFAFDYTGY DASLSPAWFE ALKMVLEKIG |
| v01149-pv1m | AVGCDPDLFW SKIPVLMEEK .LFAFDYTGY DASLSPAWFE ALKMVLEKIG |
| d00239-01 | AVGCDPETFW SKIPVMLDGD CIMAFDYTNY DGSIHPVWFQ ALKKVLENLS |
| pico-01 | AVGCDPETFW SKIPVMLDGD CIMAFDYTNY DGSIHPVWFQ ALKKVLENLS |
| hrv-01 | AVGCDPETFW SKIPVMLDGD CIMAFDYTNY DGSIHPVWFQ ALKKVLENLS |
| x02316-02 | AVGCDPEVFW SKIPAMLDDK CIMAFDYTNY DGSIHPIWFE ALKQVLVDLS |
| hrv-07 | AVGCDPEVFW STIPLMLXGE CLMAFDYSNY DGSLHPIWFK CLKMLLEDIG |
| dq473503-07 | AVGCDPEVFW STIPLMLDGE CLMAFDYSNY DGSLHPIWFK CLKMLLEDIG |
| hrv-08 | AVGCDPEVFW SKIPPMLGDG SVMAFDYTNY DGSLHPVWFK MLEVVLDRLG |
| pico-09 | AVGCDPETFW SKIPVMLDGE CIMAFDYTNY DGSIHPVWFE ALKEILKELK |
| hrv-09 | AVGCDPETFW SKIPVMLDGE CIMAFDYTNY DGSIHPVWFE ALKEILKELK |
| hrv-09-f01 | AVGCDPETFW SKIPVMLDGE CIMAFDYTNY DGSIHPVWFE ALKEILKELK |
| hrv-09-f02 | AVGCDPETFW SKIPVMLDGE CIMAFDYTNY DGSIHPVWFE ALKEILKELK |
| dq473498-10 | AVGCDPETFW SKIPVMLDGE CIMAFDYTNY DGSIHPMWFQ ALKEVLSNLS |
| hrv-10 | AVGCDPETFW SKIPVMLDGE CIMAFDYTNY DGSIHPMWFQ ALKEVLSNLS |
| ef173414-11 | AVGCDPETFW SKIPVMLDGE CIMAFDYTNY DGSIHPIWFD ALKIVLANLS |
| ef173415-12 | AVGCDPDVFW SKIPLMLDGD CLMAFDYSNY DGSLSPVWFQ ALEKVLNNLG |
| hrv-13 | AVGCDPETFW SKIPVMLDGD CIMAFDYTNY DGSIHPIWFE ALKQVLINLS |
| hrv-13-f03 | AVGCDPETFW SKIPVMLDGD CIMAFDYTNY DGSIHPIWFE ALKQVLRNLS |
| dq473493-15 | AVGCDPETFW SKIPVMLDGD CIMAFDYTNY DGSIHPIWFE ALKMILTNLH |
| l24917-16 | AVGCDPETFW SKIPLMLDGD CIMAFDYTNY DGSIHPIWFK ALGMVLDNLS |
| hrv-18 | AVGCDPETFW SKIPVMLDGE CIMAFDYTNY DGSIHPIWFQ ALKEVLSNLG |
| hrv-19 | AVGCDPEVFW SKIPVMLDGD CIMAFDYTNY DGSIHPVWFE ALKMILENLQ |
| hrv-20 | AVGCDPEVFW SKIPVMLDGD CLMAFDYTNY DGSLNPVWFK LLSIVLNDLG |
| hrv-21 | AVGCDPEVFW SKIPVMLDGD CIMAFDYTNY DGSIHPIWFQ ALKQVLTNLS |
| hrv-22 | AVGCDPETFW SKIPVMLDGD CIMAFDYTNY DGSIHPMWFE ALKMILHNLE |
| dq473497-23 | AVGCDPETFW SKIPAMLDDK CIMAFDYTNY DGSIHPIWFE ALKQVLTDLS |
| hrv-24 | AVGCDPETFW SKIPVMLDGE CIMAFDYTNY DGSIHPIWFQ ALKEVLSNLG |
| ef173416-24 | AVGCDPETFW SKIPVMLDGE CIMAFDYTNY DGSIHPIWFQ ALKEVLSNLG |
| hrv-25 | AVGCDPETFW SKIPVMLDGE CIMAFDYTNY DGSIHPIWFQ ALKEVLINLS |
| dq473508-28 | AVGCDPEVFW SKVPVMLDGE CLMAFDYTNY DGSLNPVWFK LLNKVLTDLG |
| hrv-29 | AVGCDPETFW SKIPVMLDGE CIMAFDYTNY DGSIHPIWFQ ALKEVLANLS |
| dq473512-30 | AVGCDPETFW SKIPAMLDDK CIMAFDYTNY DGSIHPIWFE ALKLVLSDLS |
| hrv-30 | AVGCDPETFW SKIPAMLDDK CIMAFDYTNY DGSIHPIWFE ALKLVLSDLS |
| hrv-31 | AVGCDPETFW SKIPVMLNGE CIMAFDYTNY DGSIHPIWFE ALKEVLSNLG |
| hrv-32 | AVGCDPETFW SKIPVMLDGD CIMAFDYTNY DGSIHPVWFE ALKEILKDLQ |
| hrv-33 | AVGCDPETFW SKIPVMLDGE CIMAFDYTNY DGSIHPVWFD ALKLVLANLS |
| hrv-34 | AVGCDPETFW SKIPVMLDGD CIMAFDYTNY DGSIHPIWFQ ALKEVLGNLS |
| dq473501-34 | AVDCDPETFW SKIPVMLDGD CIMAFDYTNY DGSIHPIWFQ ALKEVLGNLS |
| dq473505-36 | AVGCDPEIFW STIPLMLDGE CLMAFDYSNY DGSLHPVWFK CLSMLLEDIG |
| hrv-38 | AVGCDPETFW SKIPVMLDGE CIMAFDYTNY DGSIHPIWFQ ALKEVLSNLG |
| dq473495-38 | AVGCDPETFW TKIPVMLDGE CIMAFDYTNY DGSIHPIWFQ ALKEVLSNLG |
| ay751783-39 | AVGCDPETFW SKIPAMLDDK CIMAFDYTNY DGSIHPVWFQ ALKQVLSDLS |
| hrv-40 | AVGCDPETFW SKIPVMLDGE CIMAFDYTNY DGSIHPIWFQ ALKAVLSNLG |
| dq473491-41 | AVGCDPETFW SKIPVMLDGE CIMAFDYTNY DGSIHPVWFE ALKQVLANLS |
| hrv-43 | AVGCDPETFW SKIPVMLDGE CIMAFDYTNY DGSIHPVWFQ ALKSVLENLQ |
| dq473499-44 | AVGCDPETFW SKIPVMLDGE CIMAFDYTNY DGSIHPIWFQ ALKEVLANLS |
| hrv-45 | AVGCDPETFW SKIPPMLGDG CVMAFDYTNY DGSLHPIWFR LLERVLDRLG |
| dq473506-46 | AVGCDPEIFW SKIPVMLDGD CLMAFDYSNY DGSLNPVWFE LLEKVLNDLG |
| hrv-47 | AVGCDPETFW SKIPVMLDGE CIMAFDYTNY DGSIHPIWFE ALKEVLSNLG |
| dq473496-49 | AVGCDPETFW SKIPAMLDDK CIMAFDYTNY DGSIHPIWFE ALKQVLMDLS |
| hrv-49-f04 | AVGCDPEIFW SKIPAMLDDK CIMAFDYTNY DGSIHPIWFE ALKQVLMDLS |
| hrv-50 | AVGCDPETFW SKIPVMLDGE CIMAFDYTNY DGSIHPIWFQ ALKEVLNNLS |
| hrv-51 | AVGCDPEVFW SKIPVMLDGE CLMAFDYSNY DGSLNPVWFE LLAKVLDDLG |
| dq473507-53 | AVGCDPEVFW SKIPVMLDGD CLMAFDYSNY DGSLNPVWFE LLERVLNDLG |
| hrv-54 | AVGCDPETFW SKIPVMLDGE CIMAFDYTNY DGSIHPIWFQ ALKEVLSNLG |
| hrv-54-f05 | AVGCDPETFW SKIPVMLDGE CIMAFDYTNY DGSIHPIWFQ ALKEVLSNLG |
| dq473511-55 | AVGCDPEVFW SKIPVMLDGD CIMAFDYTNY DGSIHPIWFQ ALKEVLTNLS |
| hrv-56 | AVGCDPETFW SKIPVMLDGE CIMAFDYTNY DGSIHPMWFQ ALKEVLSNLS |
| hrv-57 | AVGCDPEVFW SKIPVMLDGD CIMAFDYTNY DGSIHPIWFQ ALKEVLVNLS |
| hrv-58 | AVGCDPEVFW STIPLMLDGE CLMAFDYSNY DGSLHPVWFK CLSMLLEDIG |
| dq473500-59 | GVGCDPETFW SKIPVMLDGE CIMAFDYTNY DGSIHPVWFQ ALKLVLENLG |
| hrv-60 | AVGCDPEIFW SKIPAMLNGE CLMAFDYTNY DGSIHPMWFQ ALKEILSNLG |
| hrv-61 | AVGCDPETFW SKIPVMLDGE CIMAFDYTNY DGSIHPIWFE ALKQVLINLS |
| hrv-62 | AVGCDPETFW SKIPVMLDGE CIMAFDYTNY DGSIHPIWFQ ALKEVLINLS |
| hrv-63 | AVGCDPETFW SKIPVMLDGE CIMAFDYTNY DGSIHPMWFQ ALKEVLSNLS |
| hrv-64 | AVGCDPETFW SKIPVMLDGE CIMAFDYTNY DGSIHPMWFE ALKMILHNLE |
| ef173417-64 | AVGCDPETFW SKIPVMLDGE CIMAFDYTNY DGSIHPMWFE ALKMILHNLE |
| hrv-65 | AVGCDPETFW SKIPVMLDGE CLMAFDYSNY DGSLNPVWFK LLAQILENLG |
| hrv-66 | AVGCDPETFW SKIPVMLDGE CIMAFDYTNY DGSIHPIWFQ ALKEVLSNLN |
| hrv-67 | AVGCDPETFW SKIPVMLDGD CIMAFDYTNY DGSIHPVWFD ALKEVLKDLQ |
| hrv-68 | AVGCDPEVFW SKVPVMLDGE CLMAFDYTNY DGSLNPVWFK LLNRVLTELG |
| hrv-71 | AVGCDPEVFW SKIPVMLDGE CLMAFDYSNY DGSLNPVWFK LLAGILSDLG |
| dq473492-73 | AVGCDPETFW SKIPVMLDGE CIMAFDYTNY DGSIHPIWFE ALKQVLNNLS |
| dq473494-74 | AVGCDPETFW SKIPVMLDGD CIMAFDYTNY DGSIHPVWFE ALKMVLTNLQ |
| dq473510-75 | AVGCDPETFW SKIPVMLDGD CIMAFDYTNY DGSIHPIWFE ALKQVLNNLS |
| dq473502-76 | AVGCDPETFW SKIPVMLDGE CIMAFDYTNY DGSIHPIWFD ALKLVLSNPS |
| hrv-76 | AVGCDPETFW SKIPVMLDGE CIMAFDYTNY DGSIHPIWFD ALKLVLSNLS |
| hrv-77 | AVGCDPETFW SKIPAMLDDE CIMAFDYTNY DGSIHPVWFQ ALKEILSNLG |
| hrv-78 | AVGCDPDTFW SKIPLMLDGE CLMAFDYSNY DGSLNPVWFQ ALKEILQNLG |
| ef173418-78 | AVGCDPDTFW SKIPLMLDGE CLMAFDYSNY DGSLNPVWFQ ALKEILQNLG |
| hrv-80 | AVGCDPETFW SKIPVMLDGD CLMAFDYSNY DGSLNPVWFK LLEGVLNELG |
| hrv-81 | AVGCDPETFW SKIPLMLDGD CIMAFDYTNY DGSIHPVWFK ALRMVLDNLS |
| hrv-81-f06 | AVGCDPETFW SKIPLMLDGD CIMAFDYTNY DGSIHPVWFK ALRMVLDNLS |
| hrv-81-f07 | AVGCDPETFW SKIPLMLDGD CIMAFDYTNY DGSIHPVWFK ALRMVLDNLS |
| hrv-82 | AVGCDPETFW SKIPVMLDGE CIMAFDYTNY DGSIHPMWFE ALKMVLNNLQ |
| dq473509-82 | AVGCDPETFW SKIPVMLDGE CIMAFDYTNY DGSIHPMWFE ALKMVLSNLQ |
| hrv-85 | AVGCDPETFW SKIPVMLDGE CIMAFDYTNY DGSIHPIWFQ ALKEVLSNLG |
| pico-85 | AVGCDPETFW SKIPVMLDGE CIMAFDYTNY DGSIHPIWFQ ALKEVLSNLG |
| dq473504-88 | AVGCDPEVFW STIPLMLDGE CLMAFDYSNY DGSLNPIWFK CLKMLLEDIG |
| a10937-89 | AVGCDPEVFW STIPLMLDGE CLMAFDYSNY DGSLHPVWFK CLSMLLEDIG |
| hrv-89 | AVGCDPEVFW STIPLMLDGE CLMAFDYSNY DGSLHPVWFK CLSMLLEDIG |
| hrv-89-f08 | AVGCDPEVFW STIPLMLDGE CLMAFDYSNY DGSLHPVWFK CLSMLLEDIG |
| hrv-89-f09 | AVGCDPEVFW STIPLMLDGE CLMAFDYSNY DGSLHPVWFK CLSMLLEDIG |
| m16248-89 | AVGCDPEVFW STIPLMLDGE CLMAFDYSNY DGSLHPVWFK CLSMLLEDIG |
| hrv-90 | AVGCDPETFW SKIPVMLDGE CIMAFDYTNY DGSIHPIWFQ ALKEVLTNLG |
| hrv-94 | AVGCDPETFW SKIPVMLDGE CIMAFDYTNY DGSIHPMWFE ALKMILHNLE |
| ef173419-94 | AVGCDPETFW SKIPVMLDGE CIMAFDYTNY DGSIHPMWFE ALKMILHNLE |
| hrv-95 | AVGCDPEVFW SKIPPMLGDG SVMAFDYTNY DGSLHPVWFK MLEVVLDRLG |
| hrv-96 | AVGCDPETFW SKIPVMLDGE CIMAFDYTNY DGSIHPVWFE ALKQVLLNLS |
| hrv-98 | AVGCDPETFW SKIPVMLDGE CIMAFDYTNY DGSIHPVWFQ ALKEVLSNLG |
| hrv-100 | AVGCDPETFW SKIPVMLDGE CIMAFDYTNY DGSIHPIWFQ ALKEVLTNLS |
| ef186077-qpm | AIGCDPDIFW SQLYASMEKH .LLVFDYTNY DGSLHPVWFQ ALEQLLNNLG |
| ef077279-nat001 | AIGCDPDIFW SQLYASMEKH .LLVFDYTNY DGSLHPVWFQ ALEQLLNNLG |
| ny1078 | AIGCDPDIFW SQLYSSMEKH .LLVFDYTNY DGSLHPVWFQ ALEQLLNNLG |
| ef582385-c024 | AVGCNPDVFW SQMHAMLDGE .LIAFDYTNY DGSLEPVWFK ALGKVLDQLG |
| ef077280-nat045 | AVGCDPDIFW SQLHASLGGH .LLAFDYTNY DGSLHPIWFL CLEKVLTNLG |
| ef582387-c026 | AIGCDPDVFW SQLYASMEKH .LLVFDYTNY DGSLHPVWFQ ALEQLLNNLG |
| ef582386-c025 | AVGCDPDVFW SQMKATMGEN .ILAFDYTNY DGSLDPIWFD ALKIVLNELG |
| dq473485-03 | AVGCDPDVFW SVIPCLMDGH .LMAFDYSNF DASLSPVWFE CLEKVLNKLG |
| ef173422-03 | AVGCDPDVFW SVIPCLMDGH .LMAFDYSNF DASLSPVWFE CLEKVLNKLG |
| dq473490-04 | AVGCDPDTFW SVIPCLLDGE .LIAFDYSNF DASLSPVWFN CLEIVLEKLG |
| hrv-05 | AVGCDPDTFW SVIPCLLDGE .LIAFDYSNF DASLSPVWFK CLEQVLKKIG |
| dq473486-06 | AVGCDPDVFW SIIPCLMDGH .LMAFDYSNF DASLSPVWFE CLERVLAKLG |
| l05355-14 | AVGCDPDVFW SVIPCLMDGH .LMAFDYSNF DASLSPVWFV CLEKVLTKLG |
| k02121-14 | AVGCDPDVFW SVIPCLMDGH .LMAFDYSNF DASLSPVWFV CLEKVLTKLG |
| ef173420-17 | AVGCDPDTFW SIIPCLMDGQ .LMAFDYSNF DASLSPVWFK CLEQVLSKLG |
| hrv-26 | AVGCDPDTFW SVIPCLLDGE .LIAFDYSNF DASLSPVWFK CLENVLRKIG |
| hrv-27 | AVGCDPDTFW SIIPNLLDGE .LLAFDYSNF DASLSPVWFD CLELVLRKLG |
| ef173421-27 | AVGCDPDTFW SIIPNLLDGE .LLAFDYSNF DASLSPVWFD CLELVLRKLG |
| dq473487-35 | AVGCDPDTFW SVIPCLMDGH .LMAFDYSNF DASLSPVWFT CLERVLLKLG |
| hrv-35 | AVGCDPDTFW SVIPCLMDGH .LMAFDYSNF DASLSPVWFT CLERVLLKLG |
| ef173423-37 | AVGCDPDTFW SIIPCLMDGH .LMAFDYSNF DASLSPVWFE CLEKVLIKLG |
| hrv-42 | AVGCDPDTFW SVIPCLLDGE .LIAFDYSNF DASLSPVWFE CLEQVLKKIG |
| dq473488-48 | AVGCDPDTFW SVIPCLMDGQ .LMAFDYSNF DASLSPVWFN CLEQVLIKLG |
| hrv-52 | AVGCDPDTFW SVIPCLMDGQ .LMAFDYSNF DASLSPVWFR CLEQVLAKLG |
| hrv-52-f10 | AVGCDPDTFW SVIPCLMDGQ .LMAFDYSNF DASLSPVWFK CLEQVLAKLG |
| ef173424-52 | AVGCDPDTFW SVIPCLMDGQ .LMAFDYSNF DASLSPVWFR CLEQVLAKLG |
| hrv-69 | AVGCDPDTFW SVIPCLMDGQ .LMAFDYSNF DASLSPVWFK CLEQVLTKLG |
| dq473489-70 | AVGCDPDTFW SIIPCLMDGQ .LMAFDYSNF DASLSPVWFK CLEQVLSRLG |
| hrv-72 | AVGCDPDVFW SVIPCLMDGH .LMAFDYSNF DASLSPVWFV CLEKVLYKLG |
| hrv-79 | AVGCDPDVFW SVIPCLMDGH .LMAFDYSNF DASLSPVWFE CLEKVLEKLG |
| hrv-83 | AVGCDPDVFW SVIPCLMDGH .LMAFDYSNF DASLSPVWFE CLERVLEKIG |
| hrv-84 | AVGCDPDVFW SVIPCLLDGE .LLAFDYSNF DASLSPIWFD CLELVLRKIG |
| hrv-86 | AVGCDPDVFW SVIPCLMEGH .LMAFDYSNF DASLSPVWFD CLEKVLEKLG |
| hrv-91 | AVGCDPDTFW SIIPCLMDGQ .LMAFDYSNF DASLSPVWFK CLEQVLSKLG |
| hrv-92 | AVGCDPDVFW SVIPCLMDGH .LMAFDYSNF DASLSPVWFE CLERVLSKIG |
| ef173425-93 | AVGCDPDTFW SIIPSLLDGE .LLAFDYSNF DASLSPVWFE CLELVLRKLG |
| hrv-97 | AVGCDPDTFW SVIPSLLDGE .LLAFDYSNF DASLSPVWFD CLEMVLKKLG |
| hrv-99 | AVGCDPDTFW SIIPCLLDGE .LIAFDYSNF DASLSPVWFE CLEKVLSKIG |
| | 2051 2100 |
| annotation | HHHHHHHHHH HHHBBBBB.. BBBBBB.... ....HHHHHH HHHHHHHHHH |
| af499637-cva13 | FGDRVDYIDY LNHSHHLYKN KTYCVKGGMP SGCSGTSIFN SMINNLIIRT |
| af546702-cva21 | FGSRVDYIDY LNHSHHLYKN KTYCVKGGMP SGCSGTSIFN SMINNLIIRT |
| v01149-pv1m | FGDRVDYIDY LNHSHHLYKN KTYCVKGGMP SGCSGTSIFN SMINNLIIRT |
| d00239-01 | FQ..SNLIDR LCYSKHLFKS TYYEVAGGVP SGCSGTSIFN TMINNIIIRT |
| pico-01 | FQ..SNLIDR LCYSKHLFKS TYYEVAGGVP SGCSGTSIFN TMINNIIIRT |
| hrv-01 | FQ..SNLIDR LCYSKHLFKS TYYEVAGGVP SGCSGTSIFN TMINNIIIRT |
| x02316-02 | FN..PTLIDR LCKSKHIFKN TYYEVEGGVP SGCSGTSIFN TMINNIIIRT |
| hrv-07 | FS..SQLIDQ ICNSKHIYKS KYYEVEGGMP SGCAGTSIFN TMINNIIIRV |
| dq473503-07 | FS..SQLIDQ ICNSKHIYKS KYYEVEGGMP SGCAGTSIFN TMINNIIIRV |
| hrv-08 | FP..GCAIRK LSHSAHIYKG TYYEVDGGMP SGCAGTSIFN SMINNIIIRT |
| pico-09 | FE..HRLIDR LCNSRHIFKD SYYEVEGGVP SGCSGTSIFN TMINNVIIRT |
| hrv-09 | FE..HRLIDR LCNSRHIFRD SYYEVEGGVP SGCSGTSIFN TMINNVIIRT |
| hrv-09-f01 | FE..HRLIDR LCNSRHIFRD TYYEVEGGVP SGCSGTSIFN TMINNVIIRT |
| hrv-09-f02 | FE..HRLIDR LCNSRHIFRD TYYEVEGGVP SGCSGTSIFN TMINNVIIRT |
| dq473498-10 | FE..ASLIDR LCKSKHIFKN MYYEVEGGVP SGCSGTSIFN TMINNIIIRT |
| hrv-10 | FE..ASLIDR LCKSKHIFKN MYYEVEGGVP SGCSGTSIFN TMINNIIIRT |
| ef173414-11 | FR..TDLIDR LCKSKHIFKG TYYEVEGGVP SGCSGTSIFN TMINNVIIRT |
| ef173415-12 | FP..GHLVQN LCSSKHIFRS KYYEVDGGMP SGCSGTSIFN SMINNIIIRT |
| hrv-13 | FE..DKLIDR LCRSKHIFRD TYYEVEGGVP SGCSGTSIFN TMINNIIIRT |
| hrv-13-f03 | FE..DKLIDR LCKSKHIFRD TYYEVEGGVP SGCSGTSIFN TMINNIIIRT |
| dq473493-15 | FE..PSLIDR LCNSKHIFKD TYYEVEGGVP SGCSGTSIFN TMINNVIIRT |
| l24917-16 | FN..PTLINR LCNSKHIFKS TYYEVEGGVP SGCSGTSIFN SMINNIIIRT |
| hrv-18 | FN..PNLIDR LCKSKHIFKN IYYEVEGGVP SGCSGTSIFN TMINNIIIRT |
| hrv-19 | FE..PKLIDR LCNSKHIYRD SYYEVEGGVP SGCSGTSIFN TMINNVIIRT |
| hrv-20 | FP..GELVNK LCNSKHIYKT TYYEVEGGMP SGCAGTSIFN TIINNIIIRT |
| hrv-21 | FE..ASLIDR LCKSKHIFKN IYYEVEGGVP SGCSGTSIFN TMINNVIIRT |
| hrv-22 | FE..PKLIDR LCNSKHIYRD SYYEVEGGVP SGCSGTSIFN TMINNVIIRT |
| dq473497-23 | FN..PMLIDR LCRSKHIFKN TYYEVEGGVP SGCSGTSIFN TMINNIIIRT |
| hrv-24 | FE..SSLIDR LCKSKHIFKN MYYEVEGGVP SGCSGTSIFN TMINNIIIRT |
| ef173416-24 | FE..SSLIDR LCKSKHIFKN MYYEVEGGVP SGCSGTSIFN TMINNIIIRT |
| hrv-25 | FE..PTLIDR LCRSKHIFKN TYYEVEGGVP SGCSGTSIFN TMINNIIIRT |
| dq473508-28 | FP..GDLVNK LCNSRHIYKT TYYEAEGGMP SGCAGTSIFN TIINNIIIRT |
| hrv-29 | FE..PALIDR LCRSKHIFKN TYYEVEGGIP SGCSGTSIFN TMINNVIIRT |
| dq473512-30 | FN..PMLIDR LCKSKHIFKN TYYEVEGGVP SGCSGTSIFN TMINNIIIRT |
| hrv-30 | FE..PMLIDR LCKSKHIFKN TYYEVEGGVP SGCSGTSIFN TMINNIIIRT |
| hrv-31 | FQ..SDLIDR LCRSKHIFKN VYYEVEGGVP SGCSGTSIFN TMINNIIIRT |
| hrv-32 | FE..HRLIDR LCNSKHIFKD SYYEVEGGVP SGCSGTSIFN TMINNVIIRT |
| hrv-33 | FR..TDLIDR LCKSKHIFKG TYYEVEGGVP SGCSGTSIFN TMINNVIIRT |
| hrv-34 | FE..ANLIDR LCNSKHIFKN VYYEVEGGVP SGCSGTSIFN TMINNIIIRT |
| dq473501-34 | FE..ANLIDR LCNSKHIFKN VYYEVEGGVP SGCSGTSIFN TMINNIIIRT |
| dq473505-36 | FS..SQLINQ ICNSKHIYKS KYYEVEGGMP SGCAGTSIFN TMINNIIIRT |
| hrv-38 | FQ..PNLIDR LCKSKHIFKN TYYEVEGGVP SGCSGTSIFN TMINNVIIRT |
| dq473495-38 | FQ..PNLIDR LCKSKHIFKN TYYEVEGGVP SGCSGTSIFN TMINNVIIRT |
| ay751783-39 | FD..PSLIDR LCKSKHIFRN TYYEVEGGVP SGCSGTSIFN TMMNNIIIRT |
| hrv-40 | FN..SNLIDR LCRSKHIFKN VYYEVEGGVP SGCSGTSIFN TMINNIIIRT |
| dq473491-41 | FE..DRLIDR LCKSKHIFRD TYYEVEGGVP SGCSGTSIFN TMINNIIIRT |
| hrv-43 | FE..PKLVDR LCNSKHIYRD SYYEVEGGVP SGCSGTSIFN TMINNVIIRT |
| dq473499-44 | FE..PALIDR LCRSKHIFKN TYYEVEGGIP SGCSGTSIFN TMINNVIIRT |
| hrv-45 | FP..GCAVRK LSHSTHIYKG MYYEVDGGMP SGCAGTSIFN SMINNIIIRT |
| dq473506-46 | FP..GKLVNK LCHSKHIYKT TYYEVEGGMP SGCAGTSIFN SMINNIIIRT |
| hrv-47 | FK..PNLIDR LCRSKHIFKN VYYEVEGGVP SGCSGTSIFN TMINNVIIRA |
| dq473496-49 | FD..PMLIDR LCKSKHIFRN TYYEVEGGVP SGCSGTSIFN TMINNIIIRT |
| hrv-49-f04 | FD..PMLIDR LCKSKHIFRN TYYEVEGGVP SGCSGTSIFN TMINNIIIRT |
| hrv-50 | FE..ASLIDR LCNSKHIFKN MYYEVEGGVP SGCSGTSIFN TMINNVIIRT |
| hrv-51 | FP..GNLVRK LCYSKHIFKT QYYEVEGGMP SGCAGTSIFN TIINNIIIRT |
| dq473507-53 | FP..GKLVNK LCHSKHIYKT TYYEVEGGMP SGCAGTSIFN SMINNIIIRT |
| hrv-54 | FQ..SNLIDR LCKSKHIFKN VYYEVEGGVP SGCSGTSIFN TMINNIIIRT |
| hrv-54-f05 | FQ..PNLIDR LCKSKHIFKN VYYEVEGGVP SGCSGTSIFN TMINNIIIRT |
| dq473511-55 | FE..ANLIDR LCRSKHIFKS VYYEVEGGVP SGCSGTSIFN TMINNIIIRT |
| hrv-56 | FE..ASLIDR LCKSKHIFKN MYYEVEGGVP SGCSGTSIFN TMINNIIIRT |
| hrv-57 | FE..ANLIDR LCKSKHIFKN VYYEVEGGVP SGCSGTSIFN TMINNIIIRT |
| hrv-58 | FS..SQLINQ ICNSKHIYKS KYYEVEGGMP SGCAGTSIFN TIINNIIIRT |
| dq473500-59 | FE..SGLIDR LCKSKHIFKN IYYEVEGGVP SGCSGTSIFN TMINNIIIRT |
| hrv-60 | FQ..PDLIDR LCKSKHIFKN IYYEVEGGVP SGCSGTSIFN TMINNVIIRT |
| hrv-61 | FE..HRLIDR LCKSKHIFKD TYYEVEGGVP SGCSGTSIFN TMINNVIIRT |
| hrv-62 | FE..PTLIDR LCRSKHIFKN TYYEVEGGVP SGCSGTSIFN TMINNIIIRT |
| hrv-63 | FE..ASLIDR LCNSKHIFKN MYYEVEGGVP SGCSGTSIFN TMINNIIIRT |
| hrv-64 | FE..PKLIDR LCNSKHIYRD SYYEVEGGVP SGCSGTSIFN TMINNIIIRT |
| ef173417-64 | FE..PKLIDR LCNSKHIYRD SYYEVEGGVP SGCSGTSIFN TMINNIIIRT |
| hrv-65 | FP..GNLVQK LCYSKHIFKT QYYEVEGGMP SGCAGTSIFN TMINNVIIRT |
| hrv-66 | FE..PNLIDR LCKSKHIFKN MYYEVEGGVP SGCSGTSIFN TMINNIIIRT |
| hrv-67 | FE..HRLIDR LCNSKHIFKD SYYEVEGGVP SGCSGTSVFN TMINNIIIRT |
| hrv-68 | FP..GDLVNK LCNSKHIYKT IYYEVEGGMP SGCAGTSIFN TIINNIIIRT |
| hrv-71 | FP..GDLVQK LCYSKHIFKT KYYEVEGGMP SGCAGTSIFN TMINNVIIRT |
| dq473492-73 | FE..DKLIDR LCRSKHIFRD TYYEVEGGVP SGCSGTSIFN TMINNIIIRT |
| dq473494-74 | FE..SGLIDR LCKSKHIFRD SYYEVEGGVP SGCSGTSIFN TMINNVIIRT |
| dq473510-75 | FD..SKLIDR LCNSKHIFKN SYYEVEGGIP SGCSGTSIFN TMINNIIIRT |
| dq473502-76 | FR..TDLIDR LCKSKHIFKG TYYEVEGGVP SGCSGTSIFN TMINNVIIRT |
| hrv-76 | FR..TDLIDR LCKSKHIFKG TYYEVEGGVP SGCSGTSIFN TMINNVIIRT |
| hrv-77 | FE..PGLIDR LCKSKHIFKN TYYEVEGGVP SGCSGTSIFN TMINNVIIRT |
| hrv-78 | FE..SKLIDR LCNSKHIFSS QYYEVEGGMP SGCAGTSIFN TMINNVIIRT |
| ef173418-78 | FE..SKLIDR LCNSKHIFSS QYYEVEGGMP SGCAGTSIFN TMINNVIIRT |
| hrv-80 | FQ..GDLVRK LCYSKHVYKA SYYEVEGGMP SGCAGTSIFN TMINNIIIRT |
| hrv-81 | FN..SALIDK LCNSKHIFKS TYYEVEGGVP SGCSGTSIFN SMINNIIIRT |
| hrv-81-f06 | FN..STLIDK LCNSKHIFKS TYYEVEGGVP SGCSGTSIFN SMINNIIIRT |
| hrv-81-f07 | FN..STLIDK LCNSKHIFKS TYYEVEGGVP SGCSGTSIFN SMINNIIIRT |
| hrv-82 | FE..SRLIDR LCNSKHIYKD SYYEVEGGVP SGCSGTSIFN TMINNVIIRT |
| dq473509-82 | FE..PRLIDR LCNSKHIYKD SYYEVEGGVP SGCSGTSIFN TMINNVIIRT |
| hrv-85 | FD..SNLIDR ICKSKHIFKN MYYEVEGGVP SGCSGTSIFN TMINNIIIRT |
| pico-85 | FD..SNLIDR ICKSKHIFKN MYYEVEGGVP SGCSGTSIFN TMINNIIIRT |
| dq473504-88 | FP..SQLIDQ ICNSKHIYKS KYYEVEGGMP SGCAGTSIFN TIINNIIIRT |
| a10937-89 | FS..SQLINQ ICNSKHIYKS KYYEVEGGMP SGCAGTSIFN TIINNIIIRT |
| hrv-89 | FS..SQLINQ ICNSKHIYKS KYYEVEGGMP SGCAGTSIFN TIINNIIIRT |
| hrv-89-f08 | FP..SQLINQ ICNSKHIYKS KYYEVEGGMP SGCAGTSIFN TIINNIIIRT |
| hrv-89-f09 | FS..SQLINQ ICNSKHIYKS KYYEVEGGMP SGCAGTSIFN TIINNIIIRT |
| m16248-89 | FS..SQLINQ ICNSKHIYKS KYYEVEGGMP SGCAGTSIFN TIINNIIIRT |
| hrv-90 | FE..SNLIDR LCRSKHIFKN MYYEVEGGVP SGCSGTSIFN TMINNVIIRT |
| hrv-94 | FE..PKLIDR LCNSKHIYRD SYYEVEGGVP SGCSGTSIFN TMINNVIIRT |
| ef173419-94 | FE..PKLIDR LCNSKHIYRD SYYEVEGGVP SGCSGTSIFN TMINNVIIRT |
| hrv-95 | FP..GCAVRK LSHSAHIYKG TYYEVDGGMP SGCAGTSIFN SMINNIIIRT |
| hrv-96 | FE..HRLIDR LCNSKHIFKD TYYEVEGGIP SGCSGTSIFN TMINNVIIRT |
| hrv-98 | FQ..PNLIDR LCKSKHIFKN VYYEVEGGVP SGCSGTSIFN TMINNVIIRT |
| hrv-100 | FD..ASLIDR LCKSKHIFKN MYYEVEGGVP SGCSGTSIFN TMINNIIIRT |
| ef186077-qpm | FP..GELAYK LCNTTHIYKD EIYTVKGGMP SGISGTSIFN TIINNIIIRT |
| ef077279-nat001 | LP..GELAYK LCNTTHIYKD EIYTVKGGMP SGISGTSIFN TIINNIIIRT |
| ny1078 | FP..GELAYK LCNTTHIYKD EMYSVKGGMP SGISGTSIFN TIINNIIIRT |
| ef582385-c024 | FP..GHLTQR LCNTTHIFRD TTYDVKGGMP SGISGTSIFN TMMNNIIIRT |
| ef077280-nat045 | FP..GELMGK LSNSKHIYKN AFYTTEGGMP SGICGTSIFN TMINNVIIRT |
| ef582387-c026 | FP..GELAYK LCNTTHIYKD EIYTVKGGMP SGISGTSIFN TIINNIIIRT |
| ef582386-c025 | FD..GELTEK LKKTTHVYKD EVYTVLGGMP SGTSGTSVFN TMINNIIIRT |
| dq473485-03 | FK.QPSLIQS ICNTHHIFRD EIYRVEGGMP SGCSGTSIFN SMINNIIIRT |
| ef173422-03 | FK.HPSLIQS ICNTHHIFRD EIYRVEGGMP SGCSGTSIFN SMINNIIIRT |
| dq473490-04 | FQ.CSNLIQQ ICTTHHIFRD QIYVVEGGMP SGCSGTSIFN SMINNIIIRT |
| hrv-05 | FQ.CSELIQQ ICTTHHIFKD QIYTVEGGMP SGCSGTSIFN SMINNIIIRT |
| dq473486-06 | FK.HSSLIQT ICHTHHISKD EIYIVEGGMP SGCSGTSIFN SMINNIIIRT |
| l05355-14 | FA.GSSLIQS ICNTHHIFRD EIYVVEGGMP SGCSGTSIFN SMINNIIIRT |
| k02121-14 | FA.GSSLIQS ICNTHHIFRD EIYVVEGGMP SGCSGTSIFN SMINNIIIRT |
| ef173420-17 | FK.HSNLIQT ICNTHHIFRD EIYEVEGGMP SGCSGTSVFN SMINNIIIRT |
| hrv-26 | FQ.CADMIQQ ICTTHHIFKD QIYVVEGGMP SGCSGTSIFN SMINNIIIRT |
| hrv-27 | FE.SADLVKK ISTTHHIFKD QIYVVEGGMP SGCSGTSIFN SMINNIIIRT |
| ef173421-27 | FE.SADLVKK ISTTHHIFKD QIYVVEGGMP SGCSGTSIFN SMINNIIIRT |
| dq473487-35 | FE.HSNLIQT ICNTHHIFKD EIYTVKGGMP SGCSGTSIFN SMINNIIIRT |
| hrv-35 | FE.HSNLIQT ICNTHHIFKD EIYTVKGGMP SGCSGTSIFN SMINNIIIRT |
| ef173423-37 | FK.HSSLIQS ICHTHHIFKD EIYIVEGGMP SGCSGTSIFN SMINNIIIRT |
| hrv-42 | FQ.CSELIQQ ICTTHHIFKD QIYVVEGGMP SGCSGTSIFN SMINNIIIRT |
| dq473488-48 | FK.HANLIKS ICNTHHIFRD QIYEVEGGMP SGCSGTSIFN SMINNIIIRT |
| hrv-52 | FR.HSNLIKT ICNTHHIFRD EIYEVEGGMP SGCSGTSVFN SMINNIIIRT |
| hrv-52-f10 | FK.HSNLIRT ICNTHHIFRD EIYEVEGGMP SGCSGTSVFN SMINNIIIRT |
| ef173424-52 | FR.HSNLIKT ICNTHHIFRD EIYEVEGGMP SGCSGTSVFN SMINNIIIRT |
| hrv-69 | FK.HSNLIKT ICNTHHIFRD EIYEVEGGMP SGCSGTSVFN SMINNIIIRT |
| dq473489-70 | FR.HSNLIQT ICNTHHIFRD EIYEVEGGMP SGCSGTSVFN SMINNIIIRT |
| hrv-72 | FK.HSSLIQS ICNTHHIFRD EIYVVEGGMP SGCSGTSIFN SMINNIIIRT |
| hrv-79 | FK.CASLIQT ICNTHHIFKD EIYTVKGGMP SGCSGTSIFN SMINNIIIRT |
| hrv-83 | FS.CASLIQT ICNTHHIFKD EIYTVKGGMP SGCSGTSIFN SMINNIIIRT |
| hrv-84 | FE.SANLIKQ ICTTHHIFRD QVYVVEGGMP SGCSGTSIFN SMINNIIIRT |
| hrv-86 | FK.DSNLIRS ICNTHHIFRD EIYAVRGGMP SGCSGTSIFN SMINNIIIRT |
| hrv-91 | FK.HSNLIQT ICNTHHIFRD EIYEVEGGMP SGCSGTSIFN SMINNIIIRT |
| hrv-92 | FK.CASLIQT ICNTHHIFRD EIYTVKGGMP SGCSGTSIFN SMINNIIIRT |
| ef173425-93 | FE.SADLIRK ISTTHHIFKD QIYVVEGGMP SGCSGTSIFN SMINNIIIRT |
| hrv-97 | FE.SADLIKR ISTTHHIFKD QIYVVEGGMP SGCSGTSIFN SMINNIIIRT |
| hrv-99 | FQ.CSELIQQ ICTTHHIFRD QIYTVEGGMP SGCSGTSIFN SMINNIIIRT |
| | 2101 2150 |
| annotation | HHHHHH...H HHHHBBBBBG DDBBBBB... .HHHHHHHHH HHH...HHH. |
| af499637-cva13 | LLLKTYKGID LDHLKMIAYG DDVIASYPHE VDASLLAQSG KDYGLTMTPA |
| af546702-cva21 | LLLRTYKGID LDHLKMIAYG DDVIASYPHE VDASLLAQSG KDYGLTMTPA |
| v01149-pv1m | LLLKTYKGID LDHLKMIAYG DDVIASYPHE VDASLLAQSG KDYGLTMTPA |
| d00239-01 | LVLDAYKNID LDKLKIIAYG DDVIFSYKYT LDMEAIANEG KKYGLTITPA |
| pico-01 | LVLDAYKNID LDKLKIIAYG DDVIFSYKYT LDMEAIANEG KKYGLTITPA |
| hrv-01 | LVLDAYKNID LDKLKIIAYG DDVIFSYKYT LDMEAIANEG KKYGLTITPA |
| x02316-02 | LVLDAYKNID LDKLKIIAYG DDVIFSYIHE LDMEAIAIEG VKYGLTITPA |
| hrv-07 | LVLDAYKNID LDKLKILAYG DDVIFSYNFK LDMEILAREG DKYGLTITPA |
| dq473503-07 | LVLDAYKNID LDKLKILAYG DDVIFSYNFK LDMEILAREG DKYGLTITPA |
| hrv-08 | IILHGYKNID LDQLRILTYG DDIIFTYPDK LDMSYLAQIG EQYGLKMTPA |
| pico-09 | LVLDAYKHID LDKLKIIAYG DDVIFSYKYP LDMEAIAMEG KKYGLTITPA |
| hrv-09 | LVLDAYKHID LDKLKIIAYG DDVIFSYKYP LDMEAIAMEG KKYGLTITPA |
| hrv-09-f01 | LVLDAYKNID LDKLKIIAYG DDVIFSYKYP LDMEAIAMEG KKYGLTITPA |
| hrv-09-f02 | LVLDAYKNID LDKLKIIAYG DDVIFSYKYP LDMEAIAMEG KKYGLTITPA |
| dq473498-10 | LVLDAYKNID LDKLKIIAYG DDVIFSYKYQ LDMEAIAKEG TKYGLTITPA |
| hrv-10 | LVLDAYKNID LDKLKIIAYG DDVIFSYKYQ LDMEAIAKEG TKYGLTITPA |
| ef173414-11 | LVLDAYKNID LDKLKIIAYG DDVIFSYKYE LDMEAIAKEG VKYGLTITPA |
| ef173415-12 | LVLDTYKHIN LDKLKILSYG DDVLFSYPYE LSMEDIALAG QKYGLNITPA |
| hrv-13 | LVLDAYKHID LDKLKIIAYG DDVIFSYKYP LDMEAIAIEG NKYGLTITPA |
| hrv-13-f03 | LVLDAYKHID LDKLKIIAYG DDVIFSYKYP LDMEAIATEG NKYGLTITPA |
| dq473493-15 | LVLDAYKHID LDKLKIIAYG DDVIFSYKYN LDMEVIAAEG LKYGLTITPA |
| l24917-16 | LVLDAYKHID LDKLKIIAYG DDVIFSYKYK LDMEAIAKEG QKYGLTITPA |
| hrv-18 | LVLDAYKNID LDKLKIIAYG DDVIFSYKYQ LDMEAIAKEG TKYGLTITPA |
| hrv-19 | LVLDAYKHID LDKLKIIAYG DDVIFSYKYV LDMEAIAQEG TKYGLTITPA |
| hrv-20 | LVLDTYKFIN LDKLKILAYG DDVIFSYPFD LDMKELANEG TKYGLSITPA |
| hrv-21 | LVLDAYKNID LDKLKIIAYG DDVIFSYKYQ LDMEAIANEG TKYGLTITPA |
| hrv-22 | LVLDAYKHID LDKLKIIAYG DDVIFSYKYA LDMEAIASEG TKYGLTITPA |
| dq473497-23 | LVLDAYKNID LDKLKIIAYG DDVIFSYTYE LDMEAIAIEG VKYGLTITPA |
| hrv-24 | LVLDAYKNID LDKLKIIAYG DDVIFSYKYQ LDMEAIAKEG TKYGLTITPA |
| ef173416-24 | LVLDAYKNID LDKLKIIAYG DDVIFSYKYQ LDMEAIAKEG TKYGLTITPA |
| hrv-25 | LVLDAYRNID LDKLKILAYG DDVIFSYKYQ LDMEAIAKEG IKYGLTITPA |
| dq473508-28 | LVLDTYKFIN LDKLKILAYG DDVIFSYPYD LDMKELASEG TKYGLSITPA |
| hrv-29 | LVLDAYKNID LDKLKILAYG DDVIFSYKYQ LDMEAIAKEG TKYGLTITPA |
| dq473512-30 | LVLDAYKNID LDKLKIIAYG DDVIFSYIYE LDMEAIAMEG VKYGLTITPA |
| hrv-30 | LVLDAYKNID LDKLKIIAYG DDVIFSYIYE LDMEAIAMEG VKYGLTITPA |
| hrv-31 | LVLDAYKNID LDKLKIIAYG DDVIFSYKYQ LDMEVIAKEG IKYGLTITPA |
| hrv-32 | LVLDAYKHID LDKLKIIAYG DDVIFSYKYP LEMEAIAIEG KKYGLTITPA |
| hrv-33 | LVLDAYKNID LDKLKIIAYG DDVIFSYKYQ LDMEAIALEG VKYGLTITPA |
| hrv-34 | LVLDAYKNID LDKLKIIAYG DDVIFSYKYQ LDMEAIAMEG TKYGLTITPA |
| dq473501-34 | LVLDAYKNID LDKLKIIAYG DDVIFSYKYQ LDMEAIAMEG TKYGLTITPA |
| dq473505-36 | LVLDAYKNID LDKLKILAYG DDVIFSYNFK LDMTVLAKEG EKYGLTITPA |
| hrv-38 | LVLDAYKNID LDKLKIIAYG DDVIFSYKYQ LDMEAIANEG VKYGLTITPA |
| dq473495-38 | LVLDAYKNID LDKLKIIAYG DDVIFSYKYQ LDMEAIANEG VKYGLTITPA |
| ay751783-39 | LVLDAYKNID LDKLKIIAYG DDVIFSYVYE LDMEAIAMEG KKYGLTITPA |
| hrv-40 | LVLDAYKNID LDKLKIIAYG DDVIFSYKYQ LDMEAIAKEG SKYGLTITPA |
| dq473491-41 | LVLDAYKHID LDKLKIIAYG DDVIFSYKYP LDMEAIASEG IKYGLTITPA |
| hrv-43 | LVLDAYKHID LDKLKIIAYG DDVIFSYKYT LDMEAIANEG IKYGLTITPA |
| dq473499-44 | LVLDAYKNID LDKLKILAYG DDVIFSYKYQ LDMEAIAKEG TKYGLTITPA |
| hrv-45 | IILHAYKNID LDQLRILTYG DDIIFTYPDK LDMAYLAQIG EKYGLKMTPA |
| dq473506-46 | LVLDTYKHIN LDKLKILAYG DDVLFSYPFD LDMAELAKEG DKYGLTITPA |
| hrv-47 | LVLDAYKNID LDKLKIIAYG DDVIFSYKYQ LDMEAIAKEG IKYGLTITPA |
| dq473496-49 | LVLDAYKNID LDKLKIIAYG DDVIFSYTHE LDMEAIAIEG IKYGLTITPA |
| hrv-49-f04 | LVLDAYKNID LDKLKIIAYG DDVIFSYTYE LDMEAIAMEG TKYGLTITPA |
| hrv-50 | LVLDAYKNID LDKLKIIAYG DDVIFSYKYQ LDMEAIAKEG VKYGLTITPA |
| hrv-51 | LVLDTYKYVN LDKLKILAYG DDILFSYPYD LDMKELADEG SKYGLTITPA |
| dq473507-53 | LVLDTYKYIN LDKLKILAYG DDVLFSYPYD LDMAELAKEG NKYGLTITPA |
| hrv-54 | LVLDAYKNID LDKLKIIAYG DDVIFSYKYQ LDMEAIAKEG VKYGLTITPA |
| hrv-54-f05 | LVLDAYKNID LDKLKIIAYG DDVIFSYKYQ LDMEAIAKEG VKYGLTITPA |
| dq473511-55 | LVLDAYKYID LDKLKIIAYG DDVIFSYKYQ LDMEAIAKEG TKYGLTITPA |
| hrv-56 | LVLDAYKNID LDKLKIIAYG DDVLFSYKYQ LDMEAIANEG TKYGLTITPA |
| hrv-57 | LVLDAYKNID LDKLKIIAYG DDVIFSYKYQ LDMEAIAQEG VKYGLTITPA |
| hrv-58 | LVLDAYKNID LDKLKILAYG DDVIFSYNFK LDMAVLAKEG EKYGLTITPA |
| dq473500-59 | LVLDAYKNID LDKLKIIAYG DDVIFTYKYQ LDMEAIAAEG KKYGLTITPA |
| hrv-60 | LVLDAYKNID LDKLKIIAYG DDVIFSYIYQ LDMEAIAKEG SKYGLTITPA |
| hrv-61 | LVLDAYKYID LDKLKIIAYG DDVIFSYKYP LDMEAIAAEG NKYGLTITPA |
| hrv-62 | LVLDAYKNID LDKLKILAYG DDVIFSYKYQ LDMEAIAKEG IKYGLTITPA |
| hrv-63 | LVLDAYKNID LDKLKIIAYG DDVIFSYKYQ LDMEAIAKEG TKYGLTITPA |
| hrv-64 | LVLDAYKHID LDKLKIIAYG DDVIFSYKYA LDMEAIALEG VKYGLTITPA |
| ef173417-64 | LVLDAYKHID LDKLKIIAYG DDVIFSYKYA LDMEAIALEG VKYGLTITPA |
| hrv-65 | LVLDTYKYVN LDKLKILAYG DDILFSYPYD LDMKELADEG LKYGLTITPA |
| hrv-66 | LVLDAYKNID LDKLKIIAYG DDVIFSYKYQ LDMEAIAKEG TKYGLTITPA |
| hrv-67 | LVLDAYKHID LDKLKIIAYG DDVIFSYKYP LDMEAIAMEG KKYGLTITPA |
| hrv-68 | LVLDTYKFIN LDKLKILAYG DDVIFSYPYD LDMKELASEG TKYGLSITPA |
| hrv-71 | LVLDTYKYVN LDKLKILAYG DDILFSYPYE MDMKELASEG VKYGLTITPA |
| dq473492-73 | LVLDAYKHID LDKLKIIAYG DDVIFSYKYP LDMEAIAVEG NKYGLTITPA |
| dq473494-74 | LVLDAYKHID LDKLKIIAYG DDVIFSYKYN LDMEAIAAEG LKYGLTITPA |
| dq473510-75 | LILDAYKYID LDKLKIIAYG DDIIFTYKYP LEMEAIAKEG IKYGLTITPA |
| dq473502-76 | LVLDAYKNID LDKLKIIAYG DDVIFSYKYQ LDMEAIASEG VKYGLTITPA |
| hrv-76 | LVLDAYKNID LDKLKIIAYG DDVIFSYKYQ LDMEAIASEG VKYGLTITPA |
| hrv-77 | LVLDAYKNID LDKLKIIAYG DDVIFSYKYQ LDMEAIAKEG VKYGLTITPA |
| hrv-78 | LVLDTYRHIN LDKLKILAYG DDVLFCYPYK LDMSELASEG RKYGLKITPA |
| ef173418-78 | LVLDTYRHIN LDKLKILAYG DDVLFCYPYK LDMSELASEG RKYGLKITPA |
| hrv-80 | LVLDTYKHIN LDKLKILAYG DDVLFSYPFE LDMKELASEG VKYGLTITPA |
| hrv-81 | LVLDAYKHID LDKLKIIAYG DDVIFSYKYK LDMEAIASEG QKYGLTITPA |
| hrv-81-f06 | LVLDAYKHID LDKLKIIAYG DDVIFSYKYK LDMEAIANEG QKYGLTITPA |
| hrv-81-f07 | LVLDAYKHID LDKLKIIAYG DDVIFSYKYK LDMEAIANEG QKYGLTITPA |
| hrv-82 | LVLDAYKHID LDKLKIIAYG DDVIFSYKYT LDMEAIAQEG SKYGLTITPA |
| dq473509-82 | LVLDAYKHID LDKLKIIAYG DDVIFSYKYT LDMEAIAQEG SKYGLTITPA |
| hrv-85 | LVLDAYKNID LDKLKIIAYG DDVIFSYKYQ LDMEAIAKEG VKYGLTITPA |
| pico-85 | LVLDAYKNID LDKLKIIAYG DDVIFSYKYQ LDMEAIAKEG VKYGLTITPA |
| dq473504-88 | LVLDAYKNID LDKLKILAYG DDVIFSYNFK LDMAILAKEG EKYGLTITPA |
| a10937-89 | LVLDAYKNID LDKLKILAYG DDVIFSYNFK LDMAVLAKEG EKYGLTITPA |
| hrv-89 | LVLDAYKNID LDKLKILAYG DDVIFSYNFK LDMAVLAKEG EKYGLTITPA |
| hrv-89-f08 | LVLDAYKNID LDKLKILAYG DDVIFSYNFK LDMAVLAKEG EKYGLTITPA |
| hrv-89-f09 | LVLDAYKNID LDKLKILAYG DDVIFSYNFK LDMAVLAKEG EKYGLTITPA |
| m16248-89 | LVLDAYKNID LDKLKILAYG DDVIFSYNFK LDMAVLAKEG EKYGLTITPA |
| hrv-90 | LVLDAYKNID LDKLKIIAYG DDVIFSYKYQ LDMEAIANEG KKYGLTITPA |
| hrv-94 | LVLDAYKYID LDKLKIIAYG DDVIFSYKYV LDMEAIAQEG VKYGLTITPA |
| ef173419-94 | LVLDAYKYID LDKLKIIAYG DDVIFSYKYV LDMEAIAQEG VKYGLTITPA |
| hrv-95 | IILHGYKNID LDQLRILTYG DDIIFTYPDK LDMSYLAQIG EQYGLRMTPA |
| hrv-96 | LVLDAYKHID LDKLKIIAYG DDVIFSYKYP LDMEAIALEG NKYGLTITPA |
| hrv-98 | LVLDAYKNID LDKLKIIAYG DDVIFSYKYQ LDMEAIAKEG VKYGLTITPA |
| hrv-100 | LVLDAYKNID LDKLKIIAYG DDVIFSYKYQ LDMEAIAKEG TKYGLAITPA |
| ef186077-qpm | LVLDTYKGIE LDKLKIVAYG DDVIASYPEE LDPAQIAVSG LKYGLTITPA |
| ef077279-nat001 | LVLDTYKGIE LDKLKIVAYG DDVIASYPEE LDPAQIAVSG LKYGLTITPA |
| ny1078 | LVLDTYKGIE LDKLKIVAYG DDVIASYPEE LDPAEIAISG LKYGLTITPA |
| ef582385-c024 | LVLETYKNIN LDKLRIIAYG DDVVASYPDE LDPKEIAMTA KRYGLTITPP |
| ef077280-nat045 | LVLETYKNID LDRLKIVAYG DDVIASYPEY LDPKEIAETG LKYGLTITPA |
| ef582387-c026 | LVLDTYKGIE LDKLKIVAYG DDVIASYPEE LDPAQIAISG LKYGLTITPA |
| ef582386-c025 | ITIETYKNID LDKLKIVAYG DDVVISYPDM LDAAQLADTG SKYGLTITPA |
| dq473485-03 | LILDAYKGID LDSLRILAYG DDLIVSYPFE LDSNILAAIG KNYGLTITPP |
| ef173422-03 | LILDAYKGID LDSLRILAYG DDLIVSYPFE LDSNILAAIG KNYGLTITPP |
| dq473490-04 | LVLDVYKGID LDSLKIIAYG DDVIISYPFT LDSEVIAKQG SKYGLTITPP |
| hrv-05 | LILDVYKGID LDSLRIIAYG DDVIISYPFP LDPEKIAEQG AIYGLTITPP |
| dq473486-06 | LVLDAYKGID LDKLKILAYG DDLIVSYPYE LDPEVLATLG KKYGLTITPP |
| l05355-14 | LILDAYKGID LDKLKILAYG DDLIVSYPYE LDPQVLATLG KNYGLTITPP |
| k02121-14 | LILDAYKGID LDKLKILAYG DDLIVSYPYE LDPQVLATLG KNYGLTITPP |
| ef173420-17 | LILDVYKGID LDKLKVIAYG DDLIVSYPFE LQPELLALAG KKYGLTITPP |
| hrv-26 | LILDVYKGIE LDSLRIIAYG DDVIISYPFT LDAEKISEQG LKYGLTITPP |
| hrv-27 | LILDTYKGID LDKLRIIAYG DDIIASYPFE LDSEKIAQQG AKYGLTITPP |
| ef173421-27 | LILDTYKGID LDKLRIIAYG DDIIASYPFE LDSEKIAQQG AKYGLTITPP |
| dq473487-35 | LILDAYKGID LDKLKVIAYG DDLIVSYPFE LDPGVLASHG KNYGLTITPP |
| hrv-35 | LILDAYKGID LDKLKVIAYG DDLIVSYPFE LDPGVLASHG KNYGLTITPP |
| ef173423-37 | LILDAYKGID LDKLKILAYG DDLIVSYPYE LNPEVLATLG KNYGLTITPP |
| hrv-42 | LILDVYKGIE LDSLRIIAYG DDVIVSYPFP LDPEKIAQQG VIYGLTITPP |
| dq473488-48 | LILDVYKGID LDKLKVIAYG DDLIVSYPFE LQPDLLALAG KKYGLTITPP |
| hrv-52 | LILDVYKGID LDKLKVIAYG DDLIVSYPFE LQPELLASAG KKYGLTITPP |
| hrv-52-f10 | LILDVYKGID LDKLKVIAYG DDLIVSYPFE LQPELLASAG KKYGLTITPP |
| ef173424-52 | LILDVYKGID LDKLKVIAYG DDLIVSYPFE LQPELLASAG KKYGLTITPP |
| hrv-69 | LILDVYKGID LDKLKVIAYG DDLIVSYPFE LQPELLAQAG KGYGLTITPP |
| dq473489-70 | LILDVYKGID LDKLKVIAYG DDLIVSYPFE LQPELLALAG KKYGLTITPP |
| hrv-72 | LVLDAYKGID LDKLKIIAYG DDLIVSYPYE LNPEVLATLG KSYGLTITPP |
| hrv-79 | LILDAYKGID LDKLKVIAYG DDLIVSYPFE LDPEVLATLG KNYGLVITPP |
| hrv-83 | LILDAYKGID LDKLKIIAYG DDLIASYPFE LDPEVLAKLG KNYGLTITPP |
| hrv-84 | LILDVYKGID LDKLKIIAYG DDVIISYPYE LDPERIAEQG KTYGLTITPP |
| hrv-86 | LILDAYKGID LDKLRVIAYG DDLIVSYPFE LDPSVLASMG KKYGLTITPP |
| hrv-91 | LILDVYKGID LDKLKVIAYG DDLIVSYPFE LQPELLALAG KKYGLTITPP |
| hrv-92 | LVLDAYKGID LDKLKVIAYG DDLIVSYPFE LDPGVLANLG KNYGLTITPP |
| ef173425-93 | LILDTYKGID LDKLRIIAYG DDIIASYPFE LDSEKIAQQG TKYGLTITPP |
| hrv-97 | LILDTYKGID LDKLRIIAYG DDIIASYPFE LDSEKIAQQG TKYGLTITPP |
| hrv-99 | LILDVYKGID LDSLRIIAYG DDVIVSYPFS LDPERIARQG STYGLTITPP |
| | 2151 2200 |
| annotation | HHH....... ....BB..BB BBB....... BBBB.HHHHH HHHHH..... |
| af499637-cva13 | DKSTTFETVT WENVTFLKRF FRADEKYPFL IHPVMPMKEI HESIRWTKDP |
| af546702-cva21 | DKSATFETVT WENVTFLKRF FRADEKYPFL VHPVMPMKEI HESIRWTKDP |
| v01149-pv1m | DKSATFETVT WENVTFLKRF FRADEKYPFL IHPVMPMKEI HESIRWTKDP |
| d00239-01 | DKSTEFKKLD YNNVTFLKRG FKQDEKHTFL IHPTFPVEEI YESIRWTKKP |
| pico-01 | DKSNEFKKLD YSNVTFLKRG FKQDERHTFL IHPTFPVEEI HESIRWTKKP |
| hrv-01 | DKSNEFKKLD YSNVTFLKRG FKQDERHTFL IHPTFPVEEI HESIRWTKKP |
| x02316-02 | DKSNTFVKLD YSNVTFLKRG FKQDEKYNFL IHPTFPEDEI FESIRWTKKP |
| hrv-07 | DKSSTFQELT YKNVTFLKRG FRADDRHSFL IHPTFPATEI YDSIRWTKNP |
| dq473503-07 | DKSSTFQELT YKNVTFLKRG FRADDRHSFL IHPTFPATEI YDSIRWTKNP |
| hrv-08 | DKSDTFKDLD LSTATFLKRG FKPDTHHPFL IHPTYPIQDI YESIRWTKNP |
| pico-09 | DKSDTFKKLD YDSVTFLKRG FKQDSKYPFL IHPTFPVNEI HESIRWTKKP |
| hrv-09 | DKSDTFKKLD YDSVTFLKRG FKQDSKYPFL IHPTFPVSEI HESIRWTKKP |
| hrv-09-f01 | DKSDTFKKLD YDSVTFLKRG FKQDSKYPFL IHPTFPINEI HESIRWTKKP |
| hrv-09-f02 | DKSDTFKKLD YDSVTFLKRG FKQDSKYPFL IHPTFPINEI HESIRWTKKP |
| dq473498-10 | DKSDTFKQLN YGNVTFLKRG FKQDEKYQFL IHPTFPVEEI QESIRWTKKP |
| hrv-10 | DKSDTFKQLN YGNVTFLKRG FKQDEKYQFL IHPTFPVEEI QESIRWTKKP |
| ef173414-11 | DKSNVFKKLD YNNITFLKRG FRQDDKHNFL IHPTFPIDEI HESIRWTKKP |
| ef173415-12 | DKSDKFTKID FSNATFLKRG FRMDSKHSFL VHPTFPVEEI CESIRWTKNP |
| hrv-13 | DKSDTFKKLD YDNVTFLKRG FKQDAKYPFL IHPTFPVSEI HESIRWTKKP |
| hrv-13-f03 | DKSDTFKKLD YDNVTFLKRG FKQDAKYPFL IHPTFPVSEI HESIRWTKKP |
| dq473493-15 | DKSDNFQKLT YENVTFLKRG FKQDDKYPFL IHPTFPIQEI HESIRWTKKP |
| 124917-16 | DKSSEFKELD YGNVTFLKRG FRQDDKYKFL IHPTFPVEEI YESIRWTKKP |
| hrv-18 | DKSSEFKQLD YSNVTFLKRG FRQDDKYQFL IHPTFPIEEI HESIRWTKKP |
| hrv-19 | DKSETFTKLD YSNVTFLKRG FKQDIKYPFL IHPTFPINEI HESIRWTKKP |
| hrv-20 | DKSDTFRKLD YNNATFLKRG FKQDEKYGFL IHPIYPETEV WESIRWTKNP |
| hrv-21 | DKSTCFKQLD YSNVTFLKRG FKQDEKHQFL IHPTFPIEEI HESIRWTKNP |
| hrv-22 | DKSETFKELN YDTVTFLKRG FKQDIKYPFL VHPTFPVKEI HESIRWTKKP |
| dq473497-23 | DKSTTFRKLD YNNVTFLKRG FKQDEKYSFL IHPTFPENEI FESIRWTKKP |
| hrv-24 | DKSCEFKQLD YSNVTFLKRG FRQDEKHHFL IHPTFPVEEI QESIRWTKKP |
| ef173416-24 | DKSCEFKQLD YSNVTFLKRG FRQDEKHHFL IHPTFPVEEI QESIRWTKKP |
| hrv-25 | DKSNDFKQLN YNNVTFLKRG FRQDEKHQFL IHPTFPIEEI QESIRWTKKP |
| dq473508-28 | DKSDTFKKLD YNNATFLKRG FKQDEKYSFL IHPIYPETEV WESIRWTKNP |
| hrv-29 | DKSDCFKQLN YNNVTFLKRG FRQDEKHNFL IHPTFPVEEI QESIRWTKKP |
| dq473512-30 | DKSTTFRKLD YSNVTFLKRG FKQDEKYNFL IHPTFPENEI FESIRWTKKP |
| hrv-30 | DKSTTFRKLD YSNVTFLKRG FKQDEKYNFL IHPTFPENEI FESIRWTKKP |
| hrv-31 | DKSSEFKKLD YNSVTFLKRG FKQDERYSFL IHPIFPIDEI HESIRWTKKP |
| hrv-32 | DKSDTFKKLD YDSVTFLKRG FKQDSKYPFL IHPTFPVSEI HESIRWTKKP |
| hrv-33 | DKSSIFKKLD YSNITFLKRG FRQDEKHNFL IHPTFPIDEI HESIRWTKKP |
| hrv-34 | DKSDKFKQLD YSNVTFLKRG FKQDEKHNFL IHPTFPIEEI QESIRWTKKP |
| dq473501-34 | DKSDKFKQLD YSNVTFLKRG FKQDEKHNFL IHPTFPIEEI QESIRWTKKP |
| dq473505-36 | DKSDTFQELT YKNVTFLKRG FRADERHSFL VHPTFPVAEI HDSIRWTKNP |
| hrv-38 | DKSSEFKQLD YSNVTFLKRG FKQDDRYQFL IHPTFPIDEI QESIRWTKKP |
| dq473495-38 | DKSSEFKQLD YSNVTFLKRG FKQDDRYQFL IHPTFPIDEI QESIRWTKKP |
| ay751783-39 | DKSDIFRKLD YSNVTFLKRG FRQDEKYNFL IHPTFPESEI FESIRWTKKP |
| hrv-40 | DKSNEFKQLN YNNVTFLKRG FKQDDKYQFL IHPTFPIDEI HESIRWTKKP |
| dq473491-41 | DKSDTFKKLD YDSVTFLKRG FRQDAKYPFL IHPTFPVSEI HESIRWTKKP |
| hrv-43 | DKSETFTKLD YNNVTFLKRG FKQDVKYPFL IHPTFPVQEI HESIRWTKKP |
| dq473499-44 | DKSDCFKQLD YNNVTFLKRG FRQDEKHNFL IHPTFPVEEI QESIRWTKKP |
| hrv-45 | DKSDTFKDLD LSTATFLKRG FKPDSKHPFL VHPIYPIQDI YESIRWTKNP |
| dq473506-46 | DKSDKFEKLD YGNATFLKRG FKQDNRYKFL IHPIYPESEV WESIRWTKNP |
| hrv-47 | DKSSEFKKLD YNSVTFLKRG FKQDDKYSFL IHPTFPVDEI QESIRWTKKP |
| dq473496-49 | DKSTTFVKLN YNNVTFLKRG FKQDEKYNFL IHPTFPESEI FESIRWTKKP |
| hrv-49-f04 | DKSTTFVKLN YNNVTFLKRG FKQDEKYNFL IHPTFPESEI FESIRWTKKP |
| hrv-50 | DKSENFKQLD YNNVTFLKRG FKQDEKYSFL IHPTFPIEEI QESIRWTKKP |
| hrv-51 | DKSDTFQKLD FSNATFLKRG FKPDEKHSFL VHPIYPEEEV WESIRWTKNP |
| dq473507-53 | DKSDKFEKLN YENATFLKRG FKQDDRYKFL IHPIYPESEV WESIRWTKNP |
| hrv-54 | DKSSEFKKLD YSNVTFLKRG FKQDDRYSFL IHPTFPVNEI QESIRWTKKP |
| hrv-54-f05 | DKSSEFKKLD YSNVTFLKRG FKQDERYSFL IHPTFPVNEI QESIRWTKKP |
| dq473511-55 | DKSTSFKQLD YNNVTFLKRG FKQDEKHQFL IHPTFPVEEI HESIRWTKNP |
| hrv-56 | DKSSTFKQLD YSNVTFLKRG FKQDEKYQFL IHPTFPVEEI QESIRWTKKP |
| hrv-57 | DKSTHFKQLD YNNVTFLKRG FRQDEKHQFL IHPTFPIEEI YESIRWTKNP |
| hrv-58 | DKSDVFQELT YKNVTFLKRG FRADDRHSFL IHPTFPVVEI HDSIRWTKNP |
| dq473500-59 | DKSTTFKQLD YSNVTFLKRG FKQDDKYRFL IHPTFPVEEI QESIRWTKKP |
| hrv-60 | DKSSEFKQLD YSNVTFLKRG FKQDDKYHFL IHPTFPIEEI HESIRWTKKP |
| hrv-61 | DKSDTFKKLD YSSVTFLKRG FKQDDKYSFL IHPTFPISEI HESIRWTKKP |
| hrv-62 | DKSNDFKQLD YNNVTFLKRG FRQDEKHQFL IHPTFPVEEI QESIRWTKKP |
| hrv-63 | DKSSTFTQLN YDNVTFLKRG FKQDEKHQFL IHPTFPVEEI QESIRWTKKP |
| hrv-64 | DKSETFRQLD YSNVTFLKRG FKQDAKYPFL IHPTFPIKEI HESIRWTKKP |
| ef173417-64 | DKSETFRQLD YSNVTFLKRG FKQDAKYPFL IHPTFPIKEI HESIRWTKKP |
| hrv-65 | DKSDVFKKLD FGNATFLKRG FRPDEKHSFL IHPVYPEEEV WESIRWTKNP |
| hrv-66 | DKSSEFKQLD YNNVTFLKRG FRQDERYTFL IHPTFPIEEI QESIRWTKKP |
| hrv-67 | DKSDTFKKLD YDSVTFLKRG FKQDSKYPFL IHPTFPVNEI HESIRWTKKP |
| hrv-68 | DKSDTFKKLD YSNATFLKRG FKQDEKYSFL IHPIYPETEV WESIRWTKNP |
| hrv-71 | DKSEEFSKLD FTNATFLKRN FKPDDKHSFL IHPVYPETEV WESIRWTKNP |
| dq473492-73 | DKSDTFRKLD YDNVTFLKRG FRQDAKYPFL IHPTFPVSEI HESIRWTKKP |
| dq473494-74 | DKSDCFKKLD YNTVTFLKRG FKPDDKYPFL IHPTFPIQEI HESIRWTKKP |
| dq473510-75 | DKSETFEKLD YNNVTFLKRG FKQDAKYPFL VHPTFPVQEI HESIRWTKKP |
| dq473502-76 | DKSSVFKRLD YSNITFLKRG FRQDEKHNFL IHPTFPIDEI HESIRWTKKP |
| hrv-76 | DKSSVFKRLD YSNITFLKRG FRQDEKHNFL IHPTFPIDEI HESIRWTKKP |
| hrv-77 | DKSSEFKQLD YNNVTFLKRG FRQDDKYKFL IHPTFPVDEI HESIRWTKKP |
| hrv-78 | DKSEKFIDLS YENATFLKRG FQPDQKHSFL IHPTFPISEI HNSIRWTRSP |
| ef173418-78 | DKSEKFIDLS YENATFLKRG FQPDQKHSFL IHPTFPISEI HNSIRWTRSP |
| hrv-80 | DKTEEFRKLD FGNATFLKRG FKVDDKYSFL VHPLYPEKEV WESIRWTKNP |
| hrv-81 | DKSTKFKDLD YKNVTFLKRG FRQDERYSFL IHPTFPVEEI YESIRWTKKP |
| hrv-81-f06 | DKSTEFKELD YKNVTFLKRG FRQDERYSFL IHPTFPVEEI YESIRWTKKP |
| hrv-81-f07 | DKSTEFKELD YKNVTFLKRG FRQDERYSFL IHPTFPVEEI YESIRWTKKP |
| hrv-82 | DKSETFKKLD YSNVTFLKRG FKQDERYSFL IHPTFPIDEI HESIRWTKKP |
| dq473509-82 | DKSETFKKLD YSNVTFLKRG FKQDERYPFL IHPTFPIDEI HESIRWTKKP |
| hrv-85 | DKSSEFKQLN YNNVTFLKRG FKQDDKYQFL IHPTFPIEEI QESIRWTKKP |
| pico-85 | DKSSEFKQLN YNNVTFLKRG FKQDDKYQFL IHPTFPIEEI QESIRWTKKP |
| dq473504-88 | DKSNVFQELT YNNVTFLKRG FRADEKHSFL IHPTFPVAEI YDSIRWTKNP |
| a10937-89 | DKSDVFQELT YKNVTFLKRG FRADERHSFL IHPTFPVAEI HDSIRWTKNP |
| hrv-89 | DKSDVFQELT YKNVTFLKRG FRADERHSFL IHPTFPVAEI HDSIRWTKNP |
| hrv-89-f08 | DKSDVFQELT YKNVTFLKRG FRADERHSFL VHPTFPVAEI YDSIRWTKNP |
| hrv-89-f09 | DKSDVFQELT YKNVTFLKRG FRADERHSFL VHPTFPVAEI YDSIRWTKNP |
| m16248-89 | DKSDVFQELT YKNVTFLKRG FRADERHSFL IHPTFPVAEI HDSIRWTKNP |
| hrv-90 | DKSSEFKQLS YENVTFLKRG FRQDEKHSFL IHPIFPIEEI HESIRWTKKP |
| hrv-94 | DKSDTFKELS YDNVTFLKRG FKQDTKYSFL VHPTFPVKEI HESIRWTKKP |
| ef173419-94 | DKSDTFKELS YDNVTFLKRG FKQDTKYSFL VHPTFPVKEI HESIRWTKKP |
| hrv-95 | DKSDTFKDLD LSTATFLKRG FKPDTHHPFL IHPTYPIQDI YESIRWTKNP |
| hrv-96 | DKSDTFKKLD YNSVTFLKRG FKQDDKYPFL IHPTFPISEI HESIRWTKKP |
| hrv-98 | DKSNEFKKLD YSNVTFLKRG FKQDNKYSFL IHPTFPIDEI QESIRWTKKP |
| hrv-100 | DKSNCFKQLD YDNVTFLKRG FRQDEKHQFL IHPTFPVEEI QESIRWTKKP |
| ef186077-qpm | DKSDQFVKVN WDNATFLKRG FRVDERYSFL VHPVFPEKEI FESIRWTRDP |
| ef077279-nat001 | DKSDQFVKVN WDNATFLKRG FRVDKRYNFL IHPVFPEKEI FESIRWTRDP |
| ny1078 | DKSSQFTKID WTNATFLKRG FKADEKHSFL IHPTFPESEI FESIRWTRDP |
| ef582385-c024 | DKTDQFSKMT WDNVTFLKRK FKPDSKYKFL IHPVYSMQDV YESIRWTKDP |
| ef077280-nat045 | DKSETFKPIT WETVTFLKRG FKPDEKYPFL IHPVYSMADI HESIRWTKDP |
| ef582387-c026 | DKSDHFVKVD WSNATFLKRG FRVDKKYSFL VHPVFPEKEI FESIRWTRDP |
| ef582386-c025 | DKGTDFKQVT WDTVTFLKRG FRNDLKYPFL VHPVYPMEDV FESIRWTKDP |
| dq473485-03 | DKSDAFTKIT WENITFLKRY FRPDPQFPFL IHPVMPMQDI YESIRWTRDP |
| ef173422-03 | DKSDAFTKIT WENITFLKRY FRPDPQFPFL IHPVMPMQDI YESIRWTRDP |
| dq473490-04 | DKSPNFTKMN WDNITFLKRY FKPDPDFPFL IHPVMPMADI HESIRWTKDP |
| hrv-05 | DKSPKFTKMD WNTVTFLKRH FKPDSEFPFL VHPVMPMTDI HESIRWTKDP |
| dq473486-06 | DKTEKFSKIT WDNLTFLKRY FRSDEQFPFL VHPVMPMQDI YESIRWTKDP |
| 105355-14 | DKSETFTKMT WENLTFLKRY FKPDQQFPFL VHPVMPMKDI HESIRWTKDP |
| k02121-14 | DKSETFTKMT WENLTFLKRY FKPDQQFPFL VHPVMPMKDI HESIRWTKDP |
| ef173420-17 | DKSEKFVKTT WENVTFLKRY FRPDPQYPFL IHPVMPMQDI HESIRWTRDP |
| hrv-26 | DKSPKFTKMD WHTVTFLKRH FKPDHEFPFL VHPVMPMSEI QESIRWTKDP |
| hrv-27 | DKSTQFHKIT WENVTFLKRY FRPDKQFPFL VHPVMPMSDI HESIRWTKDP |
| ef173421-27 | DKSTQFHKIT WENVTFLKRY FRPDKQFPFL VHPVMPMSDI HESIRWTKDP |
| dq473487-35 | DKSEKFEKMT WENLTFLKRY FKPDEQFPFL VHPVMPMQDI YESIRWTKDP |
| hrv-35 | DKSEKFEKMT WENLTFLKRY FKPDEQFPFL VHPVMPMQDI YESIRWTKDP |
| ef173423-37 | DKTEKFSKIT WDNLTFLKRY FRSDEQFPFL VHPVMPMQDI YESIRWTKDP |
| hrv-42 | DKSPKFTKMD WNTVTFLKRQ FKPDKEFPFL IHPVMPMSDI HESIRWTKDP |
| dq473488-48 | DKSESFSKTT WENVTFLKRY FKPDSQYPFL IHPVMPMQDI YESIRWTRDP |
| hrv-52 | DKSEKFTKTT WENVTFLKRY FRPDPQYPFL IHPVMPMQDI YESIRWTRDP |
| hrv-52-f10 | DKSEKFTKTT WENVTFLKRY FRPDPQYPFL IHPVMPMQDI HESIRWTRDP |
| ef173424-52 | DKSEKFTKTT WENVTFLKRY FRPDPQYPFL IHPVMPMQDI YESIRWTRDP |
| hrv-69 | DKSEKFTKTT WENVTFLKRY FRPDSQYPFL IHPVMPMQDI HESIRWTRDP |
| dq473489-70 | DKSEKFVKTT WENVTFLKRY FRPDPQYPFL IHPVMPMQDI HESIRWTRDP |
| hrv-72 | DKSAAFSKVT WENLTFLKRY FRPDKQFPFL VHPVMPMKDI HESIRWTKDP |
| hrv-79 | DKSEKFTKMT WENLTFLKRY FRPDEQFPFL IHPVMPMQDI YESIRWTKDP |
| hrv-83 | DKSEKFVKMT WENLTFLKRY FRPDEQFPFL IHPVMPMQDI HESIRWTKDP |
| hrv-84 | DKTEHFGKTD WSNVTFLKRN FRPDPEFPFL IHPVMPMADI HESIRWTKDP |
| hrv-86 | DKSENFTKMT WENITFLKRY FKPDKEFPFL IHPVMPMQDI HESIRWTKDP |
| hrv-91 | DKSEKFVKTT WENVTFLKRH FRPDPQYPFL IHPVMPMQDI HESIRWTRDP |
| hrv-92 | DKSEKFVKMT WENLTFLKRY FRPDEQFPFL IHPVMPMQDI HESIRWTKDP |
| ef173425-93 | DKSTQFHKVT WENVTFLKRH FRPDKQFPFL VHPVMPMTDI YESIRWTKDP |
| hrv-97 | DKSAQFHKIT WENVTFLKRY FRPDKQFPFL VHPVMPMSDI HESIRWTKDP |
| hrv-99 | DKSPEFTKMN WNNVTFLKRH FKPDSEFPFL IHPVMPMSDI YESIRWTKDP |
| | 2201 2250 |
| annotation | .HHHHHHHHH HHHHH..HHH HHHHHHHHHH .HHHHHHH.. .HHHHHHHHH |
| af499637-cva13 | RNTQDHVRSL CLLAWHNGEE EYNKFLAKIR SVPIGRALLL PEYSTLYRRW |
| af546702-cva21 | RNTQDHVRSL CLLAWHNGEE EYNKFLAKIR SVPIGRALLL PEYSTLYRRW |
| v01149-pv1m | RNTQDHVRSL CLLAWHNGEE EYNKFLAKIR SVPIGRALLL PEYSTLYRRW |
| d00239-01 | SQMQEHVLSL CHLMWHNGRK VYEDFSSKIR SVSAGRALYI PPYDLLKHEW |
| pico-01 | SQMQEHVLSL CHLMWHNGRK VYEDFSSKIR SVSAGRALYI PPYDLLKHEW |
| hrv-01 | SQMQEHVLSL CHLMWHNGRK VYEDFSSKIR SVSAGRALYI PPYDLLKHEW |
| x02316-02 | SQMHEHVLSL CHLMWHNGRD AYKKFVEKIR SVSAGRALYI PPYDLLLHEW |
| hrv-07 | SCMQEHVLSL CHLMWHNGRH EYQNFVKGIR SVSAGRALYI PAYEVLEHEW |
| dq473503-07 | SCMQEHVLSL CHLMWHNGRH EYQNFVKGIR SVSAGRALYI PAYEVLEHEW |
| hrv-08 | RCLQEHVLSL AHLCWHNGPE QYADFIGKIR SIPIGRSLYL PSYDVLLYEW |
| pico-09 | SQMQEHVLSL CHLMWHNGRD VYKEFESGIR SVS.GRALYI PPYELLLHEW |
| hrv-09 | SQMQEHVLSL CHLMWHNGRD VYKEFENGIR SVSAGRALYI PPYELLLHEW |
| hrv-09-f01 | SQMQEHVLSL CHLMWHNGRD VYKEFENGIR SVSAGRALYI PPYELLLHEW |
| hrv-09-f02 | SQMQEHVLSL CHLMWHNGRD VYKEFENGIR SVSAGRALYI PPYELLLHEW |
| dq473498-10 | SQMQEHVLSL CHLMWHNGRS VYKQFEERIR SVSAGRALYI PPYDLLLHEW |
| hrv-10 | SQMQEHVLSL CHLMWHNGRS VYKQFEERIR SVSAGRALYI PPYDLLLHEW |
| ef173414-11 | SQMQEHVLSL CHLMWHNGRD VYTQFEKRIR SVSAGRALYI PPYDLLLHEW |
| ef173415-12 | RCMQEHVLSL CYLMWHNGKQ AYEDFVSKIR SVAAGRALYI PGYEVLEHEW |
| hrv-13 | SQMQEHVLSL CHLMWHNGQD VYKEFERKIR SVSAGRALYI PPYELLLHEW |
| hrv-13-f03 | SQMQEHVLSL CHLMWHNGRD VYNEFERKIR SVSAGRALYI PPYELLLHEW |
| dq473493-15 | SQMQEHVLSL CHLMWHNGRD VYKEFEKGIR SVSAGRALYI PPYELLQHEW |
| 124917-16 | SQMQEHVLSL CHLMWHNGPE IYKDFETKIR SVSAGRALYI PPYELLRHEW |
| hrv-18 | SQMQEHVLSL CHLMWHNGKN VYKQFEQRIR SVSAGRALYI PPYELLLHEW |
| hrv-19 | SQMQEHVLSL CHLMWHNGRD EYKEFERRIR SVSAGRALYI PPYEMLLHEW |
| hrv-20 | RNMQEHVLSL CHLMWHNGKD KYKHFVDKIR SVSAGRALYI PPYEVLLHEW |
| hrv-21 | SQMQEHVLSL CHLMWHNGRD VYKQFEKRIR SVGAGRALYI PPYDLLLHEW |
| hrv-22 | SQMQEHVLSL CHLMWHNGRD VYSEFERKIR SVSAGRALYI PPYELLLHEW |
| dq473497-23 | SQMHEHVLSL CHLMWHNGRD AYKGFVEKIR SVSAGRMLYI PPYDLLLHEW |
| hrv-24 | SQMQEHVLSL CHLMWHNGRD IYKQFEQKIR SVSAGRALYV PPYDLLLHEW |
| ef173416-24 | SQMQEHVLSL CHLMWHNGRD IYKQFEQKIR SVSAGRALYV PPYDLLLHEW |
| hrv-25 | SQMQEHVLSL CHLMWHNGRD VYKKFEERIR SVGAGRALYI PPYDLLLHEW |
| dq473508-28 | RNMQEHVLSL CHLMWHNGKE KYKHFVDKIR SVSAGRALYI PPYEVLLHEW |
| hrv-29 | SQMQEHVLSL CHLMWHNGRD VYKXFEDRIR SVSAGRALYI PPYDLLLHEW |
| dq473512-30 | SQMHEHVLSL CHLMWHNGRD AYKGFVEKIR SVSAGRMLYI PPYDLLLHEW |
| hrv-30 | SQMHEHVLSL CHLMWHNGRD AYKGFVEKIR SVSAGRMLYI PPYDLLLHEW |
| hrv-31 | SQMQEHVLSL CHLMWHNGRD VYKQFEQKIR SVSAGRALYI PPYDLLLHEW |
| hrv-32 | SQMQEHVLSL CHLMWHNGRD VYKEFERGIR SVSAGRALYI PPYELLLHEW |
| hrv-33 | SQMQEHVLSL CHLMWHNGRD VYTQFERRIR SVSAGRALYI PPYDLLLHEW |
| hrv-34 | SQMQEHVLSL CHLMWHNGRD VYKQFEERIR SVSAGRALYI PPYDLLLHEW |
| dq473501-34 | SQMQEHVLSL CHLMWHNGRD VYKQFEERIR SVSAGRALYI PPYDLLLHEW |
| dq473505-36 | SCMQEHVLSL CHLMWHNGKH TYQEFVKGIR SVSAGRALYI PAYEVLEHEW |
| hrv-38 | SQMQEHVLSL CHLMWHNGKD VYKQFERKIR SVSAGRALYI PPYEVLLHEW |
| dq473495-38 | SQMQEHVLSL CHLMWHNGKD VYKQFERKIR SVSAGRALYI PPYEVLLHEW |
| ay751783-39 | SQMQEHVLSL CHLMWHNGQS AYKSFVERIR SVSAGRALYI PPYDLLLHEW |
| hrv-40 | SQMQEHVLSL CHLMWHNGKD VYKQFEQRIR SVSAGRALYI PPYELLLHEW |
| dq473491-41 | SQMQEHVLSL CHLMWHNGQD VYREFERKIR SVSAGRALYI PPYELLLHEW |
| hrv-43 | SQMQEHVLSL CHLMWHNGQD VYKEFERRIR SVSAGRALYI PPYEMLLHEW |
| dq473499-44 | SQMQEHVLSL CHLMWHDGRD VYKQFVERIR SVSAGRALYI PPYDLLLHEW |
| hrv-45 | RCLQEHVLSL AHLCWHSGPE QYADFVKRIR STSVGKNLYI PSYDVLLYEW |
| dq473506-46 | RNMQEHVLSL CHLMWHNGRD KYESFVNRIR SVSAGRALYI PPYEVLLHEW |
| hrv-47 | SQMQEHVLSL CHLMWHNGRD VYKQFEQKIR SVSAGRALYI PPYDLLLHEW |
| dq473496-49 | SQMHEHVLSL CRLMWHNGRD AYEKFVEKIR SVSAGRALYI PPYNLLLHEW |
| hrv-49-f04 | SQMHEHVLSL CHLMWHNGRD AYEKFVEKIR SVSAGRALYI PPYNLLLHEW |
| hrv-50 | SQMQEHVLSL CHLMWHNGRD VYKQFEQRIR SVGAGRALYI PPYDLLLHEW |
| hrv-51 | RNMQEHVLSL CHLMWHNGKS KYHEFVAKIR SVSAGRALYI PPFELLXHEW |
| dq473507-53 | RNMQEHVLSL CHLMWHNGKD KYDSFVNKIR SVSAGRALYI PPYELLLHEW |
| hrv-54 | SQMQEHVLSL CHLMWHNGKD VYKQFEQKIR SVGAGRALYI PPYDLLLHEW |
| hrv-54-f05 | SQMQEHVLSL CHLMWHNGRD VYKQFEQKIR SVSAGRALYI PPYDLLLHEW |
| dq473511-55 | SQMQEHVLSL CRLMWHNGRD VYKQFEKQIR SVSAGRALYI PPYDLLLHEW |
| hrv-56 | SQMQEHVLSL CHLMWHNGRS AYKQFEERIR SVSAGRALYI PPYDLLLHEW |
| hrv-57 | SQMQEHVLSL CHLMWHNGKD VYKKFEKQIR SVGAGRALYI PPYDLLLHEW |
| hrv-58 | SCMQEHVLSL CHLMWHNGRH AYQEFVKGIR SVFAGRALYI PAYEVLEHEW |
| dq473500-59 | SQMQEHVLSL CHLMWHNGRD TYKQFEDKIR SVSAGRALYI PPYDLLLHEW |
| hrv-60 | SQTQEHILSL CHLMWHNGRD VYKQFEHKIR SVSAGRALYI PPYELLQHEW |
| hrv-61 | SQMQEHVLSL CHLMWHNGRD VYKEFERKIR SVSAGRALYI PPYDLLLHEW |
| hrv-62 | SQMQEHVLSL CHLMWHNGRD VYKKFEERIR SVSAGRALYI PPYDLLLHEW |
| hrv-63 | SQMQEHVLSL CHLMWHNGRD IYKQFEERIR SVSAGRALYI PPYDLLLHEW |
| hrv-64 | SQMQXHVLSL CHLMWHNGQD VYKEFERKIR SVSAGRALYI PPYELLLHEW |
| ef173417-64 | SQMQDHVLSL CHLMWHNGQD VYKEFERKIR SVSAGRALYI PPYELLLHEW |
| hrv-65 | RNMQEHVLSL CHLMWHNGED KYQHFVAKIR SVSAGRALYI PPYELLRHEW |
| hrv-66 | SQMQEHVLSL CHLMWHNGRD AYKQFEQKIR SVSAGRALYI PPYDLLLHEW |
| hrv-67 | SQMQEHVLSL CHLMWHNGRD VYKEFERGIR SVSAGRALYI PPYELLLHEW |
| hrv-68 | RNMQEHVLSL CHLMWHNGKE KYKHFVDKIR SVSAGRALYI PPYEVLLHEW |
| hrv-71 | RNMQEHVLSL CHLMWHNGEE KYNHFVTKIR SVSAGRALYI PPYELLRHEW |
| dq473492-73 | SQMQEHVLSL CHLMWHNGRD VYKEFERKIR SVSAGRALYI PPYELLLHEW |
| dq473494-74 | SQMQEHVLSL CHLMWHNGKD VYKEFERGIR SVSAGRALYI PPYELLQHEW |
| dq473510-75 | SQMQEHVLSL CHLMWHNGPE VYKDFENKIR SVSAGRALYI PPYDLLLHEW |
| dq473502-76 | SQMQEHVLSL CHLMWHNGRD VYTQFERRIR SVSAGRALYI PPYDLLLHEW |
| hrv-76 | SQMQEHVLSL CHLMWHNGRD VYTQFERRIR SVSAGRALYI PPYDLLLHEW |
| hrv-77 | SQMQEHVLSL CHLMWHNGKD VYKQFEQKIR SVSAGRALYI PPYELLLHEW |
| hrv-78 | RNMQEHVLSL CQLMWHNGEE AYKEFVCKIR SVSAGRALYI PPFEVLRHEW |
| ef173418-78 | RNMQEHVLSL CQLMWHNGEE AYKEFVCKIR SVSAGRALYI PPFEVLRHEW |
| hrv-80 | RNMQEHVLSL CHLXWHNGKE PYNHFVAKIR SVSAGRALYI PPYEVLCHEW |
| hrv-81 | SQMQEHVLSL CHLMWHNGPE IYRDFETKIR SVSAGRALYI PPYDLLRHEW |
| hrv-81-f06 | SQMQEHVLSL CHLMWHNGPE IYRDFETKIR SVSAGRALYI PPYDLLRHEW |
| hrv-81-f07 | SQMQEHVLSL CHLMWHNGPE IYRDFETKIR SVSAGRALYI PPYDLLRHEW |
| hrv-82 | SQMQEHVLSL CHLMWHNGYE VYRDFERKIR SVSAGRALYI PPYEMLLHEW |
| dq473509-82 | SQMHEHILSL CHLMWHNGHE VYRDFERKIR SVSAGRALYI PPYEVLLHEW |
| hrv-85 | SQMQEHVLSL CHLMWHNGKD VYKQFEQKIR SVSAGRALYI PPYELLLHEW |
| pico-85 | SQMQEHVLSL CHLMWHNGKD VYKQFEQKIR SVSAGRALYI PPYELLLHEW |
| dq473504-88 | SCMQEHVLSL CHLMWHNGRH EYQSFVDRIR SVSAGRALYI PAYEVLEYEW |
| a10937-89 | SCMQEHVLSL CHLMWHNGRH AYQEFIKGIR SVSAGRALYI PAYEVLEHEW |
| hrv-89 | SCMQEHVLSL CHLMWHNGRH AYQEFIKGIR SVSAGRALYI PAYEVLEHEW |
| hrv-89-f08 | SCMQEHVLSL CHLMWHNGRH AYQEFIKGIR SVSAGRALYI PAYEVLEHEW |
| hrv-89-f09 | SCMQEHVLSL CHLMWHNGRH AYQEFTKGIR SVSAGRALYI PAYEVLEHEW |
| m16248-89 | SCMQEHVLSL CHLMWHNGRH AYQEFIKGIR SVSAGRALYI PAYEVLEHEW |
| hrv-90 | SQMQEHVLSL CHLMWHNGKD VYKQFEQKIR SVSAGRALYI PPYDVLLHEW |
| hrv-94 | SQMQEHVLSL CHLMWHNGQE EYNDFVRKIR SVSAGRALYI PPYEMLLHEW |
| ef173419-94 | SQMQEHVLSL CHLMWHNGQE EYNDFVRKIR SVSAGRALYI PPYEMLLHEW |
| hrv-95 | RCLQEHVLSL AHLCWHNGPE QYADFIGKIR SIPIGRSLYL PSYDVLLYEW |
| hrv-96 | SQMQEHVLSL CHLMWHNGRD VYKEFERKIR SVSAGRALYI PPYELLLHEW |
| hrv-98 | SQMQEHVLSL CHLMWHNGRD VYKQFEQKIR SVSAGRALYI PPYDLLLHEW |
| hrv-100 | SQMQEHVLSL CHLMWHNGRD VYKQFEDKIR SVSAGRALYI PPYDLLLHEW |
| ef186077-qpm | KNTQDHVYSL CLLMWHNGEE PYKEFISKIR KTEVGRRLYL PPYNLLERTW |
| ef077279-nat001 | KNTQDHVYSL CLLMWHNGEG PYKEFIDKIR KTEVGRRLYL PPYNLLERSW |
| ny1078 | KNTQDHVYSL CLLMWHNGEK PYREFVDRIR RTDVGRRLYL PPYSLLQRAW |
| ef582385-c024 | KNTQDHVHSL CLLAWHNGEE VYEDFCAKIR TTSVGKSLYL PPYKYLYRQW |
| ef077280-nat045 | KNTQDHVRSL CTLAWHNGEE QYNTFLSKIR TVPVGRTLSL PPYSYLRHRW |
| ef582387-c026 | KNTQDHVYSL CLLMWHNGEE PYKEFINKIR KTEVGRRLYL PPYNLLERTW |
| ef582386-c025 | RNTQDHVNSL CLLAWHNGKQ QYDDFIQKVR TTSVGRTLYL PPYEYLERRW |
| dq473485-03 | RNTQDHVRSL CMLAWHSGEK DYNDFITKIR TTDIGKCLNL PEYSVLRRRW |
| ef173422-03 | RNTQDHVRSL CMLAWHSGEK DYNDFITKIR TTDIGKCLNL PEYSVLRRRW |
| dq473490-04 | KNTQDHVRSL CMLAWHSGEQ EYNKFVEGIR STDIGKVLVI PSYSVLRRRW |
| hrv-05 | KNTQDHVRSL CMMAWHSGED EYNKFVNGIR STEIGKALLI PSYSVLRHRW |
| dq473486-06 | RNTQDHVRSL CMLAWHSGEK EYNDFIQRIR TTDIGKCLNL PEYSVLRRRW |
| 105355-14 | KNTQDHVRSL CMLAWHSGEK EYNEFIQKIR TTDIGKCLIL PEYSVLRRRW |
| k02121-14 | KNTQDHVRSL CMLAWHSGEK EYNEFIQKIR TTDIGKCLIL PEYSVLRRRW |
| ef173420-17 | KNTQDHVRSL CMLAWHSGEE EYKNFTEKIR STNIGKCLLI PEYSVLRRRW |
| hrv-26 | KNTQDHVRSL CMMAWHSGEE EYNKFVEGIR STDIGKVLLI PSYSVLRRRW |
| hrv-27 | KNTQDHVRSL CMLAWHSGEK EYDEFVRGIR STDIGKVLVI PPYSVLRHRW |
| ef173421-27 | KNTQDHVRSL CMLAWHSGEK EYDEFVRGIR STDIGKVLVI PPYSVLRHRW |
| dq473487-35 | KNTQDHVRSL CMLAWHSGQK EYEHFIERIR SVNIGRCLNL PEYSVLRKRW |
| hrv-35 | KNTQDHVRSL CMLAWHSGQK EYEHFIERIR SVNIGRCLNL PEYSVLRKRW |
| ef173423-37 | RNTQDHVRSL CMLAWHSGER EYNDFIQKIR TTDIGKCLNL PEYSVLRKRW |
| hrv-42 | KNTQDHIRSL CMMAWHSGED EYNKFVDGIR STDIGKALLI PSYSVLRRRW |
| dq473488-48 | RSTQDHVRSL CMLAWHSGEK EYKDFVDKIR STSIGKCLLI PEYSVLRRRW |
| hrv-52 | KNTQDHIRSL CMLAWHSGEE EYKNFIEKIR STSIGKCLLI PEYSVLRRRW |
| hrv-52-f10 | KNTQDHVRSL CMLAWHSGEE EYKNFTEKIR STSIGKCLLI PEYSVLRRRW |
| ef173424-52 | KNTQDHIRSL CMLAWHSGEE EYKNFIEKIR STSIGKCLLI PEYSVLRRRW |
| hrv-69 | RNTQDHIRSL CMLAWHSGEE EYKVFTDKIR STSIGKCLLI PEYSVLRRRW |
| dq473489-70 | KNTQDHVRSL CMLAWHSGEE EYKNFTEKIR STSIGKCLLI PEYSVLRRRW |
| hrv-72 | KNTQDHVRSL CMLAWHSGEE EYNEFIRKIR TTDIGKCLIL PEYSVLRKRW |
| hrv-79 | RNTQDHVRSL CMLAWHSGQK EYNEFIGKIR TTDIGKCLNL PEYSVLRRRW |
| hrv-83 | RNTQDHVRSL CMLAWHSGQK EYNEFITKIR TTEIGKCLNL PEYSVLRRRW |
| hrv-84 | KNTQDHVRSL CMLAWHSGEE EYEKFVKGIR STSVGKALVI PSYSVLRRRW |
| hrv-86 | KNTQDHVRSL CMLAWHSGES EYNDFLSKIR TTDIGKCLNL PEYSVLRRRW |
| hrv-91 | KNTQDHVRSL CMLAWHSGEE EYKNFTEKIR STNIGKCLLI PEYSVLRRRW |
| hrv-92 | RNTQDHVRSL CMLAWHSGQK EYDEFLSKIR TTDIGKCLNL PEYSVLRKRW |
| ef173425-93 | KNTQDHVRSL CMLAWHSGEK EYDKFVQGIR STDIGKVLVI PPYSVLRHRW |
| hrv-97 | KNTQDHVRSL CMLAWHSGEK EYDEFVRGIR STDIGKVLMI PPYSVLRHRW |
| hrv-99 | KNTQDHVRSL CMMAWHSGES EYNKFVDGIR STDIGRVLLI PSYSVLRRRW |
| | 2251 |
| annotation | HHHF. |
| af499637-cva13 | LDSF. |
| af546702-cva21 | LDSF. |
| v01149-pv1m | LDSF. |
| d00239-01 | YEKF. |
| pico-01 | YEKF. |
| hrv-01 | YEKF. |
| x02316-02 | YEKF. |
| hrv-07 | YEKF. |
| dq473503-07 | YEKF. |
| hrv-08 | YEKF. |
| pico-09 | YEKF. |
| hrv-09 | YEKF. |
| hrv-09-f01 | YEKF. |
| hrv-09-f02 | YEKF. |
| dq473498-10 | YEKF. |
| hrv-10 | YEKF. |
| ef173414-11 | YEKF. |
| ef173415-12 | YDKF. |
| hrv-13 | YEKF. |
| hrv-13-f03 | YEKF. |
| dq473493-15 | YEKF. |
| l24917-16 | YEKF. |
| hrv-18 | YEKF. |
| hrv-19 | YEKF. |
| hrv-20 | YEKF. |
| hrv-21 | YEKF. |
| hrv-22 | YEKF. |
| dq473497-23 | YEKF. |
| hrv-24 | YEKF. |
| ef173416-24 | YEKF. |
| hrv-25 | YEKF. |
| dq473508-28 | YEKF. |
| hrv-29 | YEKF. |
| dq473512-30 | YEKF. |
| hrv-30 | YEKF. |
| hrv-31 | YEKF. |
| hrv-32 | YEKF. |
| hrv-33 | YEKF. |
| hrv-34 | YEKF. |
| dq473501-34 | YEKF. |
| dq473505-36 | YEKF. |
| hrv-38 | YEKF. |
| dq473495-38 | YEKF. |
| ay751783-39 | YEKF. |
| hrv-40 | YEKF. |
| dq473491-41 | YEKF. |
| hrv-43 | YEKF. |
| dq473499-44 | YEKF. |
| hrv-45 | YEKF. |
| dq473506-46 | YEKF. |
| hrv-47 | YEKF. |
| dq473496-49 | YEKF. |
| hrv-49-f04 | YEKF. |
| hrv-50 | YEKF. |
| hrv-51 | YEKF. |
| dq473507-53 | YEKF. |
| hrv-54 | YEKF. |
| hrv-54-f05 | YEKF. |
| dq473511-55 | YEKF. |
| hrv-56 | YEKF. |
| hrv-57 | YEKF. |
| hrv-58 | YEKF. |
| dq473500-59 | YEKF. |
| hrv-60 | YEKF. |
| hrv-61 | YEKF. |
| hrv-62 | YEKF. |
| hrv-63 | YEKF. |
| hrv-64 | YEKF. |
| ef173417-64 | YEKF. |
| hrv-65 | YEKF. |
| hrv-66 | YEKF. |
| hrv-67 | YEKF. |
| hrv-68 | YEKF. |
| hrv-71 | YEKF. |
| dq473492-73 | YEKF. |
| dq473494-74 | YEKF. |
| dq473510-75 | YEKF. |
| dq473502-76 | YEKF. |
| hrv-76 | YEKF. |
| hrv-77 | YEKF. |
| hrv-78 | YDKF. |
| ef173418-78 | YDKF. |
| hrv-80 | YDKF. |
| hrv-81 | YEKF. |
| hrv-81-f06 | YEKF. |
| hrv-81-f07 | YEKF. |
| hrv-82 | YEKF. |
| dq473509-82 | YEKF. |
| hrv-85 | YEKF. |
| pico-85 | YEKF. |
| dq473504-88 | YEKF. |
| a10937-89 | YEKF. |
| hrv-89 | YEKF. |
| hrv-89-f08 | YEKF. |
| hrv-89-f09 | YEKF. |
| m16248-89 | YEKF. |
| hrv-90 | YEKF. |
| hrv-94 | YEKF. |
| ef173419-94 | YEKF. |
| hrv-95 | YEKF. |
| hrv-96 | YEKF. |
| hrv-98 | YEKF. |
| hrv-100 | YEKF. |
| ef186077-qpm | IDKFI |
| ef077279-nat001 | IDKFI |
| ny1078 | IDKFI |
| ef582385-c024 | IDLFI |
| ef077280-nat045 | LDLFI |
| ef582387-c026 | IDKFI |
| ef582386-c025 | IDKFI |
| dq473485-03 | LDLF. |
| ef173422-03 | LDLF. |
| dq473490-04 | LDLF. |
| hrv-05 | LDLF. |
| dq473486-06 | LDLF. |
| l05355-14 | LDLF. |
| k02121-14 | LDLF. |
| ef173420-17 | LDLF. |
| hrv-26 | LDLF. |
| hrv-27 | LELF. |
| ef173421-27 | LELF. |
| dq473487-35 | LDMF. |
| hrv-35 | LDMF. |
| ef173423-37 | LDLF. |
| hrv-42 | LDLF. |
| dq473488-48 | LDLF. |
| hrv-52 | LDLF. |
| hrv-52-f10 | LDLF. |
| ef173424-52 | LDLF. |
| hrv-69 | LDLF. |
| dq473489-70 | LDLF. |
| hrv-72 | LDLF. |
| hrv-79 | LDMF. |
| hrv-83 | LDMF. |
| hrv-84 | LDLF. |
| hrv-86 | LDMF. |
| hrv-91 | LDLF. |
| hrv-92 | LDMF. |
| ef173425-93 | LELF. |
| hrv-97 | LEMF. |
| hrv-99 | LDLF. |

ANNOTATION
HRV protein beg-end positions (cleavage sites) within this alignment.
0001-2255 polyprotein
0001-0069 VP4
0070-0345 VP2
0346-0587 VP3
0588-0919 VP1
0920-1068 2A
1069-1167 2B
1168-1499 2C
1500-1586 3A
1587-1609 3B
1610-1792 3C
1793-2255 3D
Structure features, where known are indicated on annotation line:
H = helix,
B = beta,
P = puff,
K = knob.
If structure reference has assigned letters to designate sheets or helices, these are indicated on this line.

What is claimed is:

1. A method of detecting a human rhinovirus in a sample, comprising detecting, in the sample, a nucleotide sequence selected from the group consisting of SEQ ID NOs: 1-80, thereby detecting the human rhinovirus in the sample.

2. A method of identifying a serotype of a human rhinovirus in a sample, comprising obtaining a nucleotide sequence of the human rhinovirus and comparing the nucleotide sequence to at least one of SEQ ID NOs: 1-80, wherein a match between the nucleotide sequence and one of SEQ ID NOs: 1-80 identifies the serotype of the human rhinovirus.
